(19) United States
(12) Patent Application Publication
(10) Pub. No.: US 2020/0149053 A1
(43) Pub. Date: May 14, 2020

(54) MICROORGANISMS PROGRAMMED TO PRODUCE IMMUNE MODULATORS AND ANTI-CANCER THERAPEUTICS IN TUMOR CELLS

(71) Applicant: Synlogic Operating Company, Inc., Cambridge, MA (US)

(72) Inventors: Adam Fisher, Cambridge, MA (US); Ning Li, Winchester, MA (US); Jose M. Lora, Boston, MA (US)

(21) Appl. No.: 16/619,010

(22) PCT Filed: Jul. 11, 2018
(86) PCT No.: PCT/US2018/041705

§ 371 Date: Dec. 3, 2019

Related U.S. Application Data
(63) Continuation-in-part of application No. PCT/US18/12698, filed on Jan. 5, 2018.
(60) Provisional application No. 62/531,784, filed on Jul. 12, 2017.
(60) Provisional application No. 62/543,322, filed on Aug. 9, 2017.
(60) Provisional application No. 62/552,319, filed on Aug. 30, 2017.
(60) Provisional application No. 62/592,317, filed on Nov. 29, 2017.
(60) Provisional application No. 62/607,210, filed on Dec. 18, 2017.
(60) Provisional application No. 62/628,786, filed on Feb. 9, 2018.
(60) Provisional application No. 62/642,535, filed on Mar. 13, 2018.
(60) Provisional application No. 62/657,487, filed on Apr. 13, 2018.
(60) Provisional application No. 62/688,852, filed on Jun. 22, 2018.

(51) Int. Cl. C12N 15/70; C12N 9/24; C12N 9/10; C12N 9/48; A61K 35/74; A61P 35/00; C12N 15/90; C12N 1/20

(52) U.S. Cl. CPC C12N 15/70; C12N 9/2497; C12N 9/104; C12N 1/20; A61K 35/74; A61P 35/00; C12N 15/90; C12N 9/485

(57) ABSTRACT

Genetically programmed microorganisms, such as bacteria or virus, pharmaceutical compositions thereof, and methods of modulating and treating cancers are disclosed.

### RELATED APPLICATIONS

The instant application claims priority to U.S. Provisional Application No. 62/531,784, filed on Jul. 12, 2017; U.S. Provisional Application No. 62/543,322, filed on Aug. 9, 2017; U.S. Provisional Application No. 62/552,319, filed on Aug. 30, 2017; U.S. Provisional Application No. 62/592,317, filed on Nov. 29, 2017; U.S. Provisional Application No. 62/607,210, filed on Dec. 18, 2017; PCT Application No. PCT/US2018/012698, filed on Jan. 5, 2018; U.S. Provisional Application No. 62/628,786, filed on Feb. 9, 2018; U.S. Provisional Application No. 62/642,535, filed on Mar. 13, 2018; U.S. Provisional Application No. 62/657,487, filed on Apr. 13, 2018; and U.S. Provisional Application No. 62/688,852, filed on Jun. 22, 2018. The entire contents of each of the foregoing applications are expressly incorporated by reference herein in their entireties.

### SEQUENCE LISTING

The instant application contains a Sequence Listing which has been submitted electronically in ASCII format and is hereby incorporated by reference in its entirety. Said ASCII copy, created on Jul. 10, 2018, is named 126046-31320_SL.txt and is 1,784,310 bytes in size.

### BACKGROUND OF THE INVENTION

Current cancer therapies typically employ the use of immunotherapy, surgery, chemotherapy, radiation therapy, or some combination thereof (American Cancer Society). While these drugs have shown great benefits to cancer patients, many cancers remain difficult to treat using conventional therapies. Currently, many conventional cancer therapies are administered systemically and adversely affect healthy tissues, resulting in significant side effects. For example, many cancer therapies focus on activating the immune system to boost the patient's anti-tumor response (Kong et al., 2014). However, despite such therapies, the microenvironment surrounding tumors remains highly immune suppressive. In addition, systemic altered immunoregulation provokes immune dysfunction, including the onset of opportunistic autoimmune disorders and immune-related adverse events.

Major efforts have been made over the past few decades to develop cytotoxic drugs that specifically target cancer cells. In recent years there has been a paradigm shift in oncology in which the clinical problem of cancer is considered not only to be the accumulation of genetic abnormalities in cancer cells but also the tolerance of these abnormal cells by the immune system. Consequently, recent anti-cancer therapies have been designed specifically to target the immune system rather than cancer cells. Such therapies aim to reverse the cancer immunotolerance and stimulate an effective antitumor immune response. For example, current immunotherapies include immunostimulatory molecules that are pattern recognition receptor (PRR) agonists or immunostimulatory monoclonal antibodies that target various immune cell populations that infiltrate the tumor microenvironment. However, despite their immune-targeted design, these therapies have been developed clinically as if they were conventional anticancer drugs, relying on systemic administration of the immunotherapeutic (e.g., intravenous infusions every 2-3 weeks). As a result, many current immunotherapies suffer from toxicity due to a high dosage requirement and also often result in an undesired autoimmune response or other immune-related adverse events.

Thus, there is an unmet need for effective cancer therapies that are able to target poorly vascularized, hypoxic tumor regions specifically target cancerous cells, while minimally affecting normal tissues and boost the immune systems to fight the tumors, including avoiding or reversing the cancer immunotolerance.

### SUMMARY

The present disclosure provides compositions, methods, and uses of microorganisms that selectively target tumors and tumor cells and are able to produce one or more immune modulator(s), e.g., immune initiators or combinations of one or more immune initiators and/or one or more sustainers, which are produced locally at the tumor site. In certain aspects, the present disclosure provides microorganisms, that are engineered to produce one or more immune modulator(s), e.g., immune initiators and/or sustainers. In certain aspects, the engineered microorganism is a bacteria, e.g., Salmonella typhimurium, Escherichia coli Nissle, Clostridium novyi NT, and Clostridium butyricum miyairi, as well as other exemplary bacterial strains provided herein, are able to selectively home to tumor microenvironments. Thus, in certain embodiments, the engineered microorganisms are administered systemically, e.g., via oral administration, intravenous injection, subcutaneous injection, intra tumor injection or other means, and are able to selectively colonize a tumor site.

In one aspect, disclosed herein is a modified microorganism capable of producing at least one immune initiator. In one aspect, disclosed herein is a modified microorganism capable of producing at least one immune sustainer. In one aspect, disclosed herein is a modified microorganism capable of producing at least one immune initiator and at least one immune sustainer.

In another aspect, disclosed herein is a composition comprising an immune initiator, e.g., a cytokine, chemokine, single chain antibody, ligand, metabolic converter, T cell co-stimulatory receptor, T cell co-stimulatory receptor ligand, engineered chemotherapy, or lytic peptide; and a first modified microorganism capable of producing at least one immune sustainer. In yet another aspect, disclosed herein is a composition comprising an immune sustainer, e.g., a chemokine, a cytokine, a single chain antibody, a ligand, a metabolic converter, a T cell co-stimulatory receptor, or a T cell co-stimulatory receptor ligand; and a first modified microorganism capable of producing at least one immune initiator. In another aspect, disclosed herein is a composition comprising a first modified microorganism capable of producing at least one immune initiator and at least a second modified microorganism capable of producing at least one immune sustainer.

In one embodiment, the immune initiator is capable of enhancing oncolysis, activating antigen presenting cells (APCs), and/or priming and activating T cells. In another embodiment, the immune initiator is capable of enhancing oncolysis. In another embodiment, the immune intiator is capable of activating APCs. In yet another embodiment, the immune initiator is capable of priming and activating T cells.

In one embodiment, the immune initiator is a therapeutic molecule encoded by at least one gene. In one embodiment, the immune initiator is a therapeutic molecule produced by an enzyme encoded by at least one gene. In one embodiment, the immune imitator is at least one enzyme of a biosynthetic pathway or a catabolic pathway encoded by at least one gene. In one embodiment, the immune imitator is at least one therapeutic molecule produced by at least one enzyme of a biosynthetic pathway or a catabolic pathway encoded by at least one gene. In one embodiment, the immune imitator is a nucleic acid molecule that mediates RNA interference, microRNA response or inhibition, TLR response, antisense gene regulation, target protein binding, or gene editing.

In one embodiment, the immune imitator is a cytokine, a chemokine, a single chain antibody, a ligand, a metabolic converter, a T cell co-stimulatory receptor, a T cell co-stimulatory receptor ligand, an engineered chemotherapy, or a lytic peptide. In one embodiment, the immune initiator is a secreted peptide or a displayed peptide.

In one embodiment, the immune initiator is a STING agonist, arginine, 5-FU, TNFα, IFNγ, IFNβ1, agonistic anti-CD40 antibody, CD40L, SIRPα, GMCSF, agonistic anti-OXO40 antibody, OXO40L, agonistic anti-4-1BB antibody, 4-1BBL, agonistic anti-GITR antibody, GITRL, anti-PD1 antibody, anti-PDL1 antibody, or azurin. In one embodiment, the immune initiator is a STING agonist. In one embodiment, the immune initiator is at least one enzyme of an arginine biosynthetic pathway. In one embodiment, the immune initiator is arginine. In one embodiment, the immune initiator is 5-FU. In one embodiment, the immune initiator is TNFα. In one embodiment, the immune initiator is IFNγ. In one embodiment, the immune initiator is IFNβ1. In one embodiment, the immune initiator is an agonistic anti-CD40 antibody. In one embodiment, the immune initiator is SIRPα. In one embodiment, the immune initiator is CD40L. In one embodiment, the immune initiator is GMCSF. In one embodiment, the immune initiator is an agonistic anti-OXO40 antibody. In another embodiment, the immune initiator is OXO40L. In one embodiment, the immune initiator is an agonistic anti-4-1BB antibody. In one embodiment, the immune initiator is 4-1BBL. In one embodiment, the immune initiator is an agonistic anti-GITR antibody. In another embodiment, the immune initiator is GITRL. In one embodiment, the immune initiator is an anti-PD1 antibody. In one embodiment, the immune initiator is an anti-PDL1 antibody. In one embodiment, the immune initiator is azurin.

In one embodiment, the immune initiator is a STING agonist. In one embodiment, the STING agonist is c-diAMP. In one embodiment, the STING agonist is c-GAMP. In one embodiment, the STING agonist is c-diGMP.

In one embodiment, the modified microorganism comprises at least one gene sequence encoding an enzyme which produces the immune initiator. In one embodiment, the at least one gene sequence encoding the immune initiator is a dacA gene sequence. In one embodiment, the at least one gene sequence encoding the immune initiator is a cGAS gene sequence. In one embodiment, the cGAS gene sequence is a human cGAS gene sequence. In one embodiment, the cGAS gene sequence is selected from a human cGAS gene sequence a Verminephrobacter eiseniae cGAS gene sequence, Kingella denitrificans cGAS gene sequence, and a Neisseria bacilliformis cGAS gene sequence.

In one embodiment, the at least one gene sequence encoding the immune initiator is integrated into a chromosome of the modified microorganism. In one embodiment, the at least one gene sequence encoding the immune initiator is present on a plasmid. In one embodiment, the at least one gene sequence encoding the immune initiator is operably linked to an inducible promoter. In one embodiment, the inducible promoter is induced by low oxygen, anaerobic, or hypoxic conditions.

In one embodiment, the immune initiator is arginine. In another embodiment, the immune intiator is at least one enzyme of an arginine biosynthetic pathway.

In one embodiment, the microorganism comprises at least one gene sequence encoding the at least one enzyme of the arginine biosynthetic pathway. In one embodiment, the at least one gene sequence encoding the at least one enzyme of the arginine biosynthetic pathway comprises feedback resistant argA. In one embodiment, the at least one gene sequence encoding the at least one enzyme of the arginine biosynthetic pathway is selected from the group consisting of: argA, argB, argC, argD, argE, argF, argG, argH, argI, argJ, carA, and carB. In one embodiment, the microorganism further comprises a deletion or a mutation in an arginine repressor gene (argR). In one embodiment, the at least one gene sequence for the production of arginine is integrated into a chromosome of the modified microorganism. In one embodiment, the at least one gene sequence for the production of arginine is present on a plasmid. In one embodiment, the at least one gene sequence for the production of arginine is operably linked to an inducible promoter. In one embodiment, the inducible promoter is induced by low oxygen, anaerobic, or hypoxic conditions.

In one embodiment, the immune initiator is 5-FU.

In one embodiment, the microorganism comprises at least one gene sequence encoding an enzyme capable of converting 5-FC to 5-FU. In one embodiment, the at least one gene sequence is codA. In one embodiment, the at least one gene sequence is integrated into a chromosome of the modified microorganism. In another embodiment, the at least one gene sequence is present on a plasmid. In one embodiment, the at least one gene sequence encoding the immune initiator is operably linked to an inducible promoter. In one embodiment, the inducible promoter is an FNR promoter.

In one embodiment, the immune sustainer is capable of enhancing trafficking and infiltration of T cells, enhancing recognition of cancer cells by T cells, enhancing effector T cell response, and/or overcoming immune suppression. In one embodiment, the immune sustainer is capable of enhancing trafficking and infiltration of T cells. In one embodiment, the immune sustainer is capable of enhancing recognition of cancer cells by T cells. In one embodiment, the immune sustainer is capable of enhancing effector T cell response. In one embodiment, the immune sustainer is capable of overcoming immune suppression.

In one embodiment, the immune sustainer is a therapeutic molecule encoded by at least one gene. In one embodiment, the immune sustainer is a therapeutic molecule produced by an enzyme encoded by at least one gene. In one embodiment, the immune sustainer is at least one enzyme of a biosynthetic or catabolic pathway encoded by at least one gene. In one embodiment, the immune sustainer is at least one therapeutic molecule produced by at least one enzyme of a biosynthetic or catabolic pathway encoded by at least one gene. In one embodiment, the immune sustainer is a nucleic acid molecule that mediates RNA interference, microRNA response or inhibition, TLR response, antisense gene regulation, target protein binding, or gene editing.

In one embodiment, the immune sustainer is a cytokine, a chemokine, a single chain antibody, a ligand, a metabolic converter, a T cell co-stimulatory receptor, a T cell co-stimulatory receptor ligand, or a secreted or displayed peptide.

In one embodiment, the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12. In one embodiment, the immune sustainer is a secreted peptide or a displayed peptide.

In one embodiment, the immune sustainer is a metabolic converter. In one embodiment, the metabolic converter is at least one enzyme of a kynurenine consumption pathway. In another embodiment, the metabolic converter is at least one enzyme of an adenosine consumption pathway. In another embodiment, the metabolic converter is at least one enzyme of an arginine biosynthetic pathway.

In one embodiment, the microorganism comprises at least one gene sequence encoding the at least one enzyme of the kynurenine consumption pathway. In one embodiment, the at least one gene sequence encoding the at least one enzyme of the kynurenine consumption pathway is a kynureninase gene sequence. In one embodiment, he at least one gene sequence is kynU. In one embodiment, the at least one gene sequence is operably linked to a constitutive promoter. In one embodiment, the at least one gene sequence encoding the at least one enzyme of the kynurenine consumption pathway is integrated into a chromosome of the microorganism. In another embodiment, the at least one gene sequence encoding the at least one enzyme of the kynurenine consumption pathway is present on a plasmid. In one embodiment, the microorganism comprises a deletion or a mutation in trpE.

In one embodiment, the microorganism comprises at least one gene sequence encoding at least one enzyme of an adenosine consumption pathway. In one embodiment, the at least one gene sequence encoding the at least one enzyme of the adenosine consumption pathway is selected from add, xapA, deoD, xdhA, xdhB, and xdhC. In one embodiment, the at least one gene sequence encoding the at least one enzyme of the adenosine consumption pathway is operably linked to a promoter induced by low oxygen, anaerobic, or hypoxic conditions. In one embodiment, the at least one gene sequence encoding the at least one enzyme of the adenosine consumption pathway is integrated into a chromosome of the microorganism. In another embodiment, the at least one gene sequence is present on a plasmid. In one embodiment, the modified microorganism comprises at least one gene sequence encoding an enzyme for importing adenosine into the microorganism. In one embodiment, the at least one gene sequence encoding the enzyme for importing adenosine into the microorganism is nupC or nupG.

In one embodiment, the immune sustainer is arginine. In one embodiment, the microorganism comprises at least one gene sequence encoding at least one enzyme of the arginine biosynthetic pathway. In one embodiment, the at least one gene sequence encoding at least one enzyme of the arginine biosynthetic pathway comprises feedback resistant argA. In one embodiment, the at least one gene sequence encoding the at least one enzyme of the arginine biosynthetic pathway is selected from the group consisting of: argA, argB, argC, argD, argE, argF, argG, argH, argI, argJ, carA, and carB. In one embodiment, the at least one gene sequence encoding the at least one enzyme of the arginine biosynthetic pathway is operably linked to a promoter induced by low oxygen, anaerobic, or hypoxic conditions. In one embodiment, the at least one gene sequence encoding the at least one enzyme of the arginine biosynthetic pathway is integrated into a chromosome of the modified microorganism or is present on a plasmid. In one embodiment, the microorganism further comprises a deletion or a mutation in an arginine repressor gene (argR).

In one embodiment, the immune sustainer is a STING agonist. In one embodiment, the STING agonist is c-diAMP, c-GAMP, or c-diGMP. In another embodiment, the modified microorganism comprises at least one gene sequence encoding an enzyme which produces the STING agonist. In one embodiment, the at least one gene sequence encoding the immune sustainer is a dacA gene sequence. In one embodiment, the at least one gene sequence encoding the immune sustainer is a cGAS gene sequence. In one embodiment, the cGAS gene sequence is selected from a human cGAS gene sequence, a Verminephrobacter eiseniae cGAS gene sequence, Kingella denitrificans cGAS gene sequence, and a Neisseria bacilliformis cGAS gene sequence.

In one embodiment, the immune initiator is not the same as the immune sustainer. In one embodiment, the immune initiator is different than the immune sustainer.

In one embodiment, the modified microorganism comprises at least one gene sequence encoding an enzyme capable of producing the STING agonist. In one embodiment, the at least one gene sequence encoding the STING agonist is a dacA gene. In one embodiment, the at least one gene sequence encoding the STING agonist is a cGAS gene. In one embodiment, the STING agonist is c-diAMP. In one embodiment, the STING agonist is c-GAMP. In one embodiment, the STING agonist is c-diGMP.

In one embodiment, the bacterium is an auxotroph in a gene that is not complemented when the bacterium is present in a tumor. In one embodiment, the gene that is not complemented when the bacterium is present in a tumor is a dapA gene. In one embodiment, expression of the dapA gene fine-tunes the expression of the one or more immune initiators. In one embodiment, the bacterium is an auxotroph in a gene that is complemented when the bacterium is present in a tumor. In one embodiment, the gene that is complemented when the bacterium is present in a tumor is a thyA gene.

In one embodiment, the bacterium further comprises a mutation or deletion in an endogenous prophage.

In one embodiment, the at least one gene sequence is operably linked to an inducible promoter. In one embodiment, the inducible promoter is induced by low-oxygen or anaerobic conditions. In one embodiment, the inducible promoter is induced by the hypoxic environment of a tumor. In one embodiment, the promoter is an FNR promoter.

In one embodiment, the at least one gene sequence is integrated into a chromosome in the bacterium. In one embodiment, the at least one gene sequence is located on a plasmid in the bacterium.

In one embodiment, the bacterium is non-pathogenic. In one embodiment, he bacterium is Escherichia coli Nissle.

In one aspect, disclosed herein is a modified microorganism capable of producing an effector molecule, wherein the effector molecule is selected from the group consisting of CXCL9, CXCL10, hyaluronidase, and SIRPα.

In one embodiment, the modified microorganism comprises at least one gene sequence encoding CXCL9. In one embodiment, the at least one gene sequence encoding CXCL9 is linked to an inducible promoter.

In one embodiment, the modified microorganism comprises at least one gene sequence encoding CXCL10. In one embodiment, the at least one gene sequence encoding CXCL10 is linked to an inducible promoter.

In one embodiment, the modified microorganism comprises at least one gene sequence encoding hyaluronidase. In one embodiment, the at least one gene sequence encoding hyaluronidase is linked to an inducible promoter.

In one embodiment, the modified microorganism comprises at least one gene sequence encoding the SIRPα. In one embodiment, the at least one gene sequence encoding the SIRPα is linked to an inducible promoter.

In one embodiment, the effector molecule is secreted. In another embodiment, the effector molecule is displayed on the cell surface.

In one aspect, disclosed herein is a modified microorganism capable of converting 5-FC to 5-FU. In another aspect, disclosed herein is a modified microorganism capable of converting 5-FC to 5-FU, wherein the modified microorganism is further capable of producing a STING agonist.

In one embodiment, the microorganism comprises at least one gene sequence encoding an enzyme capable of converting 5-FC to 5-FU. In one embodiment, the at least one gene sequence is codA. In one embodiment, the at least one gene sequence is a codA::upp fusion. In one embodiment, the at least one gene sequence is operably linked to an inducible promoter or a constitutive promoter. In one embodiment, the inducible promoter is a FNR promoter. In one embodiment, the at least one gene sequence is integrated into the chromosome of the microorganism or is present on a plasmid.

In one embodiment, the microorganism capable of converting 5-FC to 5-FU is further capable of producing a STING agonist. In one embodiment, the STING agonist is c-diAMP, c-GAMP, or c-diGMP. In one embodiment, the modified microorganism comprises at least one gene sequence encoding an enzyme which produces the STING agonist. In one embodiment, the at least one gene sequence encoding the enzyme which produces the STING agonist is a dacA gene sequence. In one embodiment, the at least one gene sequence encoding the enzyme which produces the STING agonist is a cGAS gene sequence. In one embodiment, the cGAS gene sequence is a human cGAS gene sequence. In one embodiment, the at least one gene sequence encoding the enzyme which produces the STING agonist is operably linked to an inducible promoter. In one embodiment, the inducible promoter is an FNR promoter. In one embodiment, the at least one gene sequence encoding the enzyme which produces the STING agonist is integrated into a chromosome of the microorganism or is present on a plasmid.

In another aspect, disclosed herein is a modified microorganism capable of secreting a dimerized IL-12, wherein the modified microorganism comprises a gene sequence comprising a p35 IL-12 subunit gene sequence linked to a p40 IL-12 subunit gene sequence by a linker sequence, and a secretion tag sequence. In one embodiment, the secretion tag sequence is selected from the group consisting of SEQ ID NO: 1235, 1146-1154, 1156, and 1168. In one embodiment, the linker sequence comprises SEQ ID NO: 1194. In one embodiment, the p35 IL-12 subunit gene sequence comprises SEQ ID NO: 1192, and wherein the p40 IL-12 subunit gene sequence comprises SEQ ID NO: 1193. In one embodiment, the gene sequence comprises a sequence selected from the group consisting of SEQ ID NOs: 1169-1179. In one embodiment, the gene sequence is operably linked to an inducible promoter. In one embodiment, the inducible promoter is an FNR promoter. In one embodiment, the gene sequence is integrated into a chromosome of the microorganism or is present on a plasmid.

In another aspect, disclosed herein is a modified microorganism capable of secreting an IL-15 fusion protein, wherein the modified microorganism comprises a sequence comprising an IL-15 gene sequence fused to a sushi domain sequence. In one embodiment, the sequence is selected from the group consisting of SEQ ID NOs: 1195-1198.

In one embodiment, the modified microorganism disclosed herein is a bacterium. In one embodiment, the modified microorganism disclosed herein is a yeast. In one embodiment, the modified microorganism is an E. coli bacterium. In one embodiment, the modified microorganism is an E. coli Nissle bacterium.

In one embodiment, the modified microorganism disclosed herein comprises at least one mutation or deletion in a gene which results in one or more auxotrophies. In one embodiment, the at least one deletion or mutation is in a dapA gene and/or a thyA gene.

In one embodiment, the modified microorganism disclosed herein comprises a phage deletion.

In one aspect, disclosed herein is a composition comprising at least a first modified microorganism capable of producing an immune initiator, and at least a second modified microorganism capable of producing an immune sustainer.

In one aspect, disclosed herein is a composition comprising an immune sustainer and at least one modified microorganism capable of producing an immune initiator. In one embodiment, the at least one modified microorganism is capable of producing both the immune intiator and the immune sustainer. In another embodiment, the at least one modified microorganism is capable of producing the immune initiator, and at least a second modified microorganism is capable of producing the immune sustainer. In yet another embodiment, the immune sustainer is not produced by a modified microorganism in the composition.

In one aspect, disclosed herein is a composition comprising an immune initiator and at least one modified microorganism capable of producing an immune sustainer. In one embodiment, the at least one modified microorganism is capable of producing both the immune intiator and the immune sustainer. In another embodiment, the at least one modified microorganism is capable of producing the immune sustainer, and at least a second modified microorganism is capable of producing the immune initiator. In yet another embodiment, the immune initiator is not produced by a modified microorganism in the composition.

In one embodiment, the immune initiator is not arginine, TNFα, IFNγ, IFNβ1, GMCSF, anti-CD40 antibody, CD40L, agonistic anti-OX40 antibody, OXO40L, agonistic anti-41BB antibody, 41BBL, agonistic anti-GITR antibody, GITRL, anti-PD1 antibody, anti-PDL1 antibody, and/or azurin. In one embodiment, the immune initiator is not arginine. In one embodiment, the immune initiator is not TNFα. In one embodiment, the immune initiator is not IFNγ. In one embodiment, the immune initiator is not IFNβ1. In one embodiment, the immune initiator is not an anti-CD40 antibody. In one embodiment, the immune initiator is not CD40L. In one embodiment, the immune initiator is not GMCSF. In one embodiment, the immune initiator is not an agonistic anti-OXO40 antibody. In one embodiment, the immune initiator is not OXO40L. In one embodiment, the immune initiator is not an agonistic anti-4-1BB antibody. In one embodiment, the immune initiator is not 4-1BBL. In one embodiment, the immune initiator is not an agonistic anti-GITR antibody. In one embodiment, the immune initiator is not GITRL. In one embodiment, the immune initiator is not an anti-PD1 antibody. In one embodiment, the immune initiator is not an anti-PDL1 antibody. In one embodiment, the immune initiator is not azurin.

In one embodiment, the immune sustainer is not at least one enzyme of a kynurenine consumption pathway, at least one enzyme of an adenosine consumption pathway, anti-PD1 antibody, anti-PDL1 antibody, anti-CTLA4 antibody, IL-15, IL-15 sushi, IFNγ, agonistic anti-GITR antibody, GITRL, an agonistic anti-OX40 antibody, OX40L, an agonistic anti-4-1BB antibody, 4-1BBL, or IL-12. In one embodiment, the immune sustainer is not at least one enzyme of a kynurenine consumption pathway. In one embodiment, the immune sustainer is not at least one enzyme of an adenosine consumption pathway. In one embodiment, the immune sustainer is not arginine. In one embodiment, the immune sustainer is not at least one enzyme of an arginine biosynthetic pathway. In one embodiment, the immune sustainer is not an anti-PD1 antibody. In one embodiment, the immune sustainer is not an anti-PDL1 antibody. In one embodiment, the immune sustainer is not an anti-CTLA4 antibody. In one embodiment, the immune sustainer is not an agonistic anti-GITR antibody. In one embodiment, the immune sustainer is not GITRL. In one embodiment, the immune sustainer is not IL-15. In one embodiment, the immune sustainer is not IL-15 sushi. In one embodiment, the immune sustainer is not IFNγ. In one embodiment, the immune sustainer is not an agonistic anti-OX40 antibody. In one embodiment, the immune sustainer is not OX40L. In one embodiment, the immune sustainer is not an agonistic anti-4-1BB antibody. In one embodiment, the immune sustainer is not 4-1BBL. In one embodiment, the immune sustainer is not IL-12.

In one aspect, disclosed herein is a pharmaceutically acceptable composition comprising a modified microorganism disclosed herein, and a pharmaceutically acceptable carrier. In one aspect, disclosed herein is a pharmaceutically acceptable composition comprising a composition disclosed herein, and a pharmaceutically acceptable carrier. In one embodiment, the composition is formulated for intratumoral injection. In another embodiment, the pharmaceutically acceptable composition is for use in treating a subject having cancer. In another embodiment, the pharmaceutically acceptable composition is for use in inducing and modulating an immune response in a subject.

In one aspect, disclosed herein is a kit comprising a pharmaceutically acceptable composition disclosed herein, and instructions for use thereof.

In one aspect, disclosed herein is a method of treating cancer in a subject, the method comprising administering to the subject a pharmaceutically acceptable composition disclosed herein, thereby treating cancer in the subject.

In one aspect, disclosed herein is a method of inducing and sustaining an immune response in a subject, the method comprising administering to the subject a pharmaceutically acceptable composition disclosed herein, thereby inducing and sustaining the immune response in the subject.

In one aspect, disclosed herein is a method of inducing and sustaining an immune response in a subject, the method comprising administering to the subject a pharmaceutically acceptable composition described herein, thereby inducing and sustaining the immune response in the subject.

In another aspect, disclosed herein is a method of inducing an abscopal effect in a subject having a tumor, the method comprising administering to the subject a pharmaceutically acceptable composition described herein, thereby inducing the abscopal effect in the subject.

In one aspect, disclosed herein is a method of inducing immunological memory in a subject having a tumor, the method comprising administering to the subject a pharmaceutically acceptable composition described herein, thereby inducing the immunological memory in the subject.

In one aspect, disclosed herein is a method of inducing partial regression of a tumor in a subject, the method comprising administering to the subject a pharmaceutically acceptable composition described herein, thereby inducing the partial regression of the tumor in the subject. In one embodiment, the partial regression is a decrease in size of the tumor by at least about 10%, at least about 25%, at least about 50%, or at least about 75%.

In one aspect, disclosed herein is a method of inducing complete regression of a tumor in a subject, the method comprising administering to the subject a pharmaceutically acceptable composition described herein, thereby inducing the complete regression of the tumor in the subject. In one embodiment, the tumor is not detectable in the subject after administration of the pharmaceutically acceptable composition.

In one aspect, disclosed herein is a method of treating cancer in a subject, the method comprising administering a first modified microorganism to the subject, wherein the first modified microorganism is capable of producing an immune initiator; and administering a second modified microorganism to the subject, wherein the second modified microorganism is capable of producing an immune sustainer, thereby treating cancer in the subject.

In one aspect, disclosed herein is a method of inducing and sustaining an immune response in a subject, the method comprising administering a first modified microorganism to the subject, wherein the first modified microorganism is capable of producing an immune initiator; and administering a second modified microorganism to the subject, wherein the second modified microorganism is capable of producing an immune sustainer, thereby inducing and sustaining the immune response in the subject.

In one embodiment, the administering steps are performed at the same time. In one embodiment, the administering of the first modified microorganism to the subject occurs before the administering of the second modified microorganism to the subject. In one embodiment, the administering of the second modified microorganism to the subject occurs before the administering of the first modified microorganism to the subject.

In one aspect, disclosed herein is a method of treating cancer in a subject, the method comprising administering a first modified microorganism to the subject, wherein the first modified microorganism is capable of producing an immune initiator; and administering an immune sustainer to the subject, thereby treating cancer in the subject.

In one aspect, disclosed herein is a method of inducing and sustaining an immune response in a subject, the method comprising administering a first modified microorganism to the subject, wherein the first modified microorganism is capable of producing an immune initiator; and administering an immune sustainer to the subject, thereby inducing and sustaining the immune response in the subject.

In one embodiment, the administering steps are performed at the same time. In one embodiment, the administering of the first modified microorganism to the subject occurs before the administering of the immune sustainer to the subject. In another embodiment, the administering of the immune sustainer to the subject occurs before the administering of the first modified microorganism to the subject.

In one aspect, disclosed herein is a method of treating cancer in a subject, the method comprising administering an immune initiator to the subject; and administering a first modified microorganism to the subject, wherein the first modified microorganism is capable of producing an immune sustainer, thereby treating cancer in the subject.

In one aspect, disclosed herein is a method of inducing and sustaining an immune response in a subject, the method comprising administering an immune initiator to the subject; and administering a first modified microorganism to the subject, wherein the first modified microorganism is capable of producing an immune sustainer, thereby inducing and sustaining the immune response in the subject.

In one embodiment, the administering steps are performed at the same time. In one embodiment, the administering of the first modified microorganism to the subject occurs before the administering of the immune initiator to the subject. In one embodiment, the administering of the immune initiator to the subject occurs before the administering of the first modified microorganism to the subject.

In one embodiment, the administering is intratumoral injection.

Accordingly, the disclosure provides compositions comprising one or more modified bacteria comprising gene sequence(s) encoding one or more immune modulators. In some embodiments, the immune modulator is an immune initiator, which may for example modulate, e.g., promote tumor lysis, antigen presentation by dendritic cells or macrophages, or T cell activation or priming Examples of such immune initiators include cytokines or chemokines, such as TNFα, IFN-gamma and IFN-beta1, a single chain antibodies, such as anti-CD40 antibodies, or (3) ligands such as SIRPα or CD40L, a metabolic enzymes (biosynthetic or catabolic), such as a STING agonist producing enzyme, or (5) cytotoxic chemotherapies. The immune modulators, e.g., immune initiators, may be operably linked to a promoter not associated with the gene sequence(s) in nature.

In some embodiments, the genetically engineered bacteria are capable of producing one or more STING agonist(s), such as c-di-AMP, 3′3′-cGAMP and/or c-2′3′-cGAMP. In some embodiments, the genetically engineered bacteria comprise gene sequences encoding a diadenylate cyclase, such as DacA, e.g., from Listeria monocytogenes. In some embodiments, the genetically engineered bacteria comprise gene sequences encoding a 3′3′-cGAMP synthase. Non-limiting examples of 3′3′-cGAMP synthases described in the instant disclosure include 3′3′-cGAMP synthase Verminephrobacter eiseniae (EF01-2 Earthworm symbiont), 3′3′-cGAMP synthase from Kingella denitrificans (ATCC 33394), and 3′3′-cGAMP synthase from Neisseria bacilliformis (ATCC BAA-1200). In some embodiments, the genetically engineered bacteria comprise gene sequences encoding a 2′3′-cGAMP synthase, such as human cGAS.

In some embodiments, the genetically engineered bacteria comprise gene sequences encoding agonists of co-stimulatory receptors, including but not limited to OX40, GITR, 41BB.

In some embodiments, the compositions of the disclosure comprise genetically engineered bacereia which comprise gene sequences encoding an engineered chemotherapy. One example of an engineered chemotherapy may be provide by engineered bacteria which are capable of converting 5-FC to 5-FU in the tumor setting.

In some embodiments, the composition further comprises one or more genetically engineered microorganism(s) comprising gene sequence(s) for producing an immune sustainer, which may modulate, e.g., enhance, tumor infiltration or the T cell response or modulate, e.g., alleviate, immune suppression. Such a sustainer may be selected from a cytokine or chemokine, a single chain antibody antagonistic peptide or ligand, and a metabolic enzyme pathways.

Examples of immune sustaining cytokines which may be produced by the genetically engineered bacteria include IL-15 and CXCL10, which may be secreted into the tumor microenvironment. Non-limiting examples of single chain antibodies include anti-PD-1, anti-PD-L1, or anti-CTLA-4, which may be secreted into the tumor microenvironment or displayed on the microorganism cell surface.

In some embodiments, the genetically engineered bacteria comprise gene sequences encoding circuitry for one or more metabolic conversions, i.e., the bacteria are cabable performing one or more enzyme-catalyzed reactions, which can be either biosynthetic or catabolic in nature. Accordingly, in some embodiments, the genetically engineered bacteria are capable of producing metabolites which modulate, e.g., promote or contribute to immune initiation and/or immune sustenance or are capable of consuming metabolites which modulate, e.g., promote, immune suppression. For example, in some embodiments, the compositions comprise genetically engineered bacteria that are capable of consuming the immunosuppressive metabolite kynurenine, e.g., by expressing kynureninase e.g., from Pseudomonas fluorescens. In some embodiments, the genetically engineered bacteria comprise gene sequences encoding an adenosine catabolic pathway and optionally a adenosine transporter, and are capable of breaking down the tumor growth promoting metabolite adenosine within the tumor microenvironment. In other embodiments, the genetically engineered bacteria are capable of producing arginine, a stimulator of T cell activation and priming. In some embodiments, the bacteria are cabable of consuming ammonia in the tumor microenvironment, reducing access to nitrogen which supports tumor growth.

In any of these compositions, the promoter operably linked to the gene sequences (s) for producing the immune modulator, e.g., the immune initiator and/or immune sustainer may an inducible promoter. In some embodiments, the promoter is induced by low-oxygen or anaerobic conditions, such as by the hypoxic environment of a tumor. Non-limiting examples of such low oxygen inducible promoters of the disclosure include FNR-inducible promoters, ANR-inducible promoters, and DNR-inducible promoters. In some embodiments, the promoter operably linked to the gene sequence(s) for producing the immune modulator, e.g., the immune initiator or immune sustainer, is directly or indirectly induced by a chemical inducer that is not normally present within the tumor. In some embodiments, the promoter is induced in vitro during fermentation in a suitable growth vessel. In some embodiments, the chemical inducer is selected from tetracycline, IPTG, arabinose, cumate, and salicylate.

In some embodiments, the composition comprises bacteria that are auxotrophs for a particular metabolite, e.g., the bacterium is an auxotroph in a gene that is not complemented when the microorganism(s) is present in the tumor. In some embodiments, the bacterium is an auxotroph in the DapA gene. In some embodiments, the composition comprises bacteria that are auxotrophs for a particular metabolite, e.g., the bacterium is an auxotroph in a gene that is complemented when the microorganism(s) is present in the tumor. In some embodiments, the bacterium is an auxotroph in the ThyA gene. In some embodiments, the bacterium is an auxotroph in the TrpE gene.

In some embodiments, the bacterium is a Gram-positive bacterium. In some embodiments, the bacterium is a Gram-negative bacterium. In some embodiments, the bacterium is an obligate anaerobic bacterium. In some embodiments, the bacterium is a facultative anaerobic bacterium. Non-limiting examples of bacteria contemplated in the disclosure include Clostridium novyi NT, and Clostridium butyricum, and Bifidobacterium longum. In some embodiments, the bacterim is selected from E. coli Nissle, and E. coli K-12.

In some embodiments, the bacterium comprises an antibiotic resistance gene sequence. In some embodiments, the one or more of the gene sequence(s) encoding the immune modulator(s) are present on a chromosome. In some embodiments, the one or more of the gene sequence(s) encoding the immune modulator(s) are present on a plasmid.

Additionally, pharmaceutical compositions are provided, further comprising one or more immune checkpoint inhibitors, such as CTLA-4 inhibitor, a PD-1 inhibitor, and a PD-L1 inhibitor. Such checkpoint inhibitors may be administered in combination, sequentially or concurrently with the genetically engineered bacteria.

Additionally, pharmaceutical compositions are provided, further comprising one or more agonists of co-stimulatory receptors, such as OX40, GITR, and/or 41BB, including but not limited to agonistic molecules, such as ligands or agonistic antibodies which are capable of binding to co-stimulatory receptors, such as OX40, GITR, and/or 41BB. Such agonistic molecules may be administered in combination, sequentially or concurrently with the genetically engineered bacteria.

In any of these embodiments, a combination of engineered bacteria can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion, and oncolytic viruses. In any of these embodiments, the engineered bacteria can produce one or more cytotoxins or lytic peptides. In any of these embodiments, the engineered bacteria can be used in conjunction with a cancer or tumor vaccine.

In one embodiment, disclosed herein is a modified bacterium comprising at least one an immune initiator, wherein the immune initiator is capable of producing a stimulator of interferon gene (STING) agonist.

### BRIEF DESCRIPTION OF THE FIGURES

FIG. 1 depicts a schematic showing the STING Pathway in Antigen Presenting Cells.

FIG. 2 depicts a bar graph showing extracellular and intracellular cyclic-di-AMP accumulation in vitro as measured by LC/MS (SYN3527). No cyclic-di-AMP accumulation was measured in control strains which do not contain the dacA expression construct.

FIG. 3 depicts a bar graph showing cyclic-di-AMP production upon induction of SYN3527.

FIG. 4A and FIG. 4B depict relative IFNb1 mRNA expression in RAW 267.4 cells treated with with live bacteria (FIG. 4A) and heat killed bacteria (FIG. 4B). SYN=streptomycin resistant Nissle. SYN-STING=SYN3527 comprising p15-ptet-DacA (from Listeria monocytogenes).

FIG. 5A and FIG. 5B depicts graphs showing INF-b1 production (FIG. 5A) or IFN-b1 mRNA expression (FIG. 5B) in WT or TLR4−/− mouse bone marrow derived dendritic cell cultures at 4 hours post stimulation with SYN3527 (comprising tetracycline-inducible DacA from Listeria monocytogenes). SYN3527 was either left uninduced (“STING-UN”) or induced with tetracyclin “STING-IN” prior to the experiment. TLR4−/− cells are unable to respond to LPS. Low to negative levels of IFNb in non-induced bacteria indicates that IFNb induction is dependent on expression of the STING agonist Similar levels of IFNb inducation were observed in WT and TLR4−/− demonostrating that STING agonist mediated induction of IFNb is not dependent on LPS/TLR4. FIG. 5C and FIG. 5D depicts graphs showing IL-6 mRNA expression (FIG. 5C) or CD80 mRNA expression (FIG. 5D) in WT or TLR4−/− mouse bone marrow derived dendritic cells at 4 hours post stimulation with SYN3527 (comprising tetracycline-inducible DacA from Listeria monocytogenes). SYN3527 was either left uninduced (“STING-UN”) or induced with tetracyclin “STING-IN” prior to the experiment. TLR4−/− cells are unable to respond to LPS. Levels of IL-6 and CD80 are similar upon exposure to induced SYN3527 compared to non-induced or SYN94, indicating that LPS/TLR4 signaling is likely causing the majority of the signal which results in IL-6 and CD80 upregulation.

FIG. 6A and FIG. 6B depict line graphs of an in vitro analysis of the activity of the STING agonist producing strain on IFN-beta1 induction in RAW 264.7 cells at various multiplicities of infection (MOI) at 4 hours (FIG. 6A) and at 4 hours and at 45 mins (FIG. 6B) and demonstrates that SYN3527 (comprising the tetracycline inducible dacA construct) drives dose-dependent IFN-beta1 induction in RAW 264.7 cells (immortalized murine macrophage cell line). Briefly, bacteria (WT Nissle (Labeled in graph as “SYN”) or SYN3527 (labeled in graph as “SYN-STING”; comprising tetracycline-inducible DacA from Listeria monocytogenes) were co-cultured at various multiplicities of infection (MOI) with 0.5×106 RAW 264.7 cells. SYN3527 was either left uninduced or induced with tetracycline as indicated prior to the experiment. Co-cultures were incubated for 4 hours or 45 minutes as indicated and protein extracts were analyzed.

FIG. 7A depicts a schematic showing an outline of an in vivo mouse study, the results of which are shown in FIG. 7B and FIG. 7C. FIG. 7B depicts a line graph showing the average mean tumor volume of mice implanted with B16-F10 tumors and treated with saline, SYN94 (streptomycin resistant wild type Nissle) or SYN3527 (comprising the tetracycline inducible dacA construct). FIG. 7C depicts line graphs showing tumor volume of individual mice in the study. FIG. 7D depicts a graph showing the tumor weight at day 9. FIG. 7E depicts a graph showing total T cell numbers in the tumor draining lymph node at day 9 measured via flow cytometry. FIG. 7F depicts a graph showing percentage of activated (CD44 high) T cells among CD4 (conventional) and CD8 T cell subsets and FIG. 7G depicts a graph showing a lack of activation of Tregs upon STING injection in the tumor draining lymph node at day 9 as measured via flow cytometry. FIG. 7H depicts a graph showing tumor colonization.N.D.=Not detected.

FIG. 8A and FIG. 8B depict bar graphs showing the concentration of IFN-b1 in B16 tumors measured by Luminex Bead Assay at day 2 (FIG. 8A) or day 9 (FIG. 8B) after administration and induction of tet-inducible STING Agonist producing strain SYN3527 as compared to mice treated with saline or streptomycin resistant Nissle.

FIG. 9A, FIG. 9B, and FIG. 9C show cytokine kinetic analysis of SYN-STING-treated B16F10 tumors. B16F10 tumors were treated as described herein, with cohorts of tumors harvested on days 2 and 9 post treatment initiation. Tumors were homogenized, treated with protease inhibitors and frozen for future analysis. Thawed homogenates were analyzed utilizing a custom Luminex cytokine array. Panel in FIG. 9A shows cytokines indicative of innate immune cell responses which show upregulation in response to SYN-STING treatment. Panel in FIG. 9B and FIG. 9C shows cytokines associated with cytolytic and activated effector T cells. Panel in FIG. 9D shows cytokines upregulated in response to bacterial injection. Statistical significance determined using the Holm-Sidak method adjusted for multiple T test comparing experimental groups within a cohort. Group compared to saline; * P<0.05, ** P<0.005. Group compared to SYN (WT); # P<0.05. FIG. 9A depicts bar graphs showing the concentration of IL-6 (left panel), IL-1beta (middle panel) and MCP-1 (right panel) in B16 tumors measured by Luminex Bead Assay at day 2 and 9 after administration and induction of tet-inducible STING Agonist producing strain SYN3527 as compared to mice treated with saline or streptomycin resistant Nissle. FIG. 9B depicts bar graphs showing the concentration of Granzyme B (left panel), IL-2 (middle panel) and IL-15 (right panel) in B16 tumors measured by Luminex Bead Assay at day 2 and 9 after administration and induction of tet-inducible STING Agonist producing strain SYN3527 as compared to mice treated with saline or streptomycin resistant Nissle. FIG. 9C depicts bar graphs showing the concentration of IFNg (upper panel), and IL-12p′70 (lower panel) in B16 tumors measured by Luminex Bead Assay at day 2 and 9 after administration and induction of tet-inducible STING Agonist producing strain SYN3527 as compared to mice treated with saline or streptomycin resistant Nissle. FIG. 9D depicts bar graphs showing the concentration of TNF-a (upper panel), and GM-CSF (lower panel) in B16 tumors measured by Luminex Bead Assay at day 2 and 9 after administration and induction of tet-inducible STING Agonist producing strain SYN3527 as compared to mice treated with saline or streptomycin resistant Nissle. In FIG. 9A, FIG. 9B, and FIG. 9C, bars in each panel are arranged in the same order as in FIG. 9A and FIG. 9B, i.e, saline (left), streptomycin resistant wild type Nissle (middle) and SYN3527 (SYN-STING, right).

FIG. 10A, FIG. 10B and FIG. 10C depict graphs showing in vitro analysis of SYN-STING (SYN3527) activity following co-culture with dendritic cells (DCs) and macrophages. Briefly, the ability of SYN-STING to activate the STING pathway in antigen presenting cell populations was assessed. Bacteria (WT Nissle or SYN3527 (comprising tetracycline-inducible DacA from Listeria monocytogenes). were co-cultured at various multiplicities of infection (MOI) with 0.5×106 RAW 264.7 cells (immortalized murine macrophage cell line) or murine bone-marrow-derived DCs. SYN3527 was either left uninduced (“STINGun”) or induced with tetracycline “STINGin” prior to the experiment. Co-cultures were incubated for 2 or 4 hours as indicated and protein extracts were analyzed or mRNA was harvested to measure IFNβ1 gene induction via quantitative PCR. FIG. 10A and FIG. 10B depicts graphs showing IFNβ1 (FIG. 10A) or IFN-b1 mRNA induction (FIG. 10B) in mouse bone marrow derived dendritic cells either at 4 hours post stimulation (FIG. 10A) or at 2 and 4 hours post stimulation (FIG. 10B). FIG. 10C depicts the mean IFNβ1 gene induction (mRNA levels) in RAW 264.7 cells at 2 hours. Heat-killed bacteria were generated at 60° C. for 30 min. Mean Ctrl=control PBS; LPS=100 ng/mL lipopolysaccharide. All signals normalized to PBS treated controls.

FIG. 11 depicts a line graph of an in vivo analysis showing the effect of the STING agonist producing strain on tumor volume over time at three different doses (1×10\hat{ }7, 5×10\hat{ }7 and 1×10\hat{ }8) and demonstrates that SYN3527 (comprising the tetracycline inducible Listeria monocytogenes dacA construct) drives dose-dependent tumor control in the A20 lymphoma model.

FIG. 12A, FIG. 12B, FIG. 12C, and FIG. 12D depict line graphs showing each individual mouse for the study shown in FIG. 11.

FIG. 13 depicts a line graph showing that complete regressions elicited by SYN3527 (WT Tet-STING) result in long lasting immunological memory in the A20 tumor model. In contrast to the naïve controls, secondary implants were completely rejected in the animals previously treated with SYN3527 which showed complete regression. Graph shows individual tumor measurements for the indicated experimental groups.

FIG. 14A depicts a schematic of a non-limiting example of the disclosure in which a microorganism is genetically engineered to express gene sequence(s) encoding one or more enzymes for the production of a STING agonist and additionally one or more gene sequence(s) for the expression of a kynurenine consuming enzyme. Non-limiting examples of such enzymes for the production of STING agonists include dacA, e.g., from Listeria monocytogenes. Non-limiting examples of such kynurenine consuming enzymes include kynureninase (e.g., kynureninase from Pseudomonas fluorescens). More generally, immune initiator circuits (STING agonist producer or others described herein) may be combined with immune sustainer circuits (e.g., kynurenine consumption or others described herein). FIG. 14B depicts a schematic of a graph showing one embodiment of the disclosure, in which a microorganism which is genetically engineered to express an immune initiator circuit (STING agonist) and immune sustainer circuit (kynurenine circuit) first produces high levels of immune stimulator (STING agonist producing enzyme e.g., DacA, e.g., from Listeria monocytogenes) and at a later time point produces the immune sustainer (kynureninase, e.g., from Pseudomonas fluorescens). In some embodiments, expression of the immune initiator (in this case, STING agonist producing enzyme, e.g., dacA, is induced by an inducer. In some embodiments, immune sustainer (in this case kynureninase) is induced by an inducer. In some embodiments, both immune initiator (STING agonist producing enzyme, e.g., dacA) and immune sustainer (e.g., kynureninase) are induced by one or more inducer(s). Inducer #1 (e.g., inducing immune initiator dacA expression) and inducer #2 (e.g., inducing immune sustainer kynureninase expression) may be the same or different inducers. Inducer #1 and inducer #2 may be administered sequentially or concurrently. Non-limiting examples of inducers include in vivo conditions of the gut or the tumor microenvironment (e.g., low oxygen, certain nutrients, etc.), in vitro growth conditions, or chemical inducers (e.g., arabinose, cumate, and salicylate, IPTG or other chemical inducers described herein). In other embodiments, the immune initiator (e.g., STING agonist producing enzyme, e.g., dacA) and the immune sustainer (e.g., kynureninase) are driven by constitutive promoters, including but not limited to those described herein. In some embodiments, the immune initiator (e.g., STING agonist producing enzyme, e.g., dacA) is driven by an inducible promoter and the immune sustainer (e.g., kynureninase) is driven by a constitutive promoter. In some embodiments, the immune initiator (e.g., STING agonist producing enzyme, e.g., dacA) is driven by an constitutive promoter and the immune sustainer (e.g., kynureninase) is driven by an inducible promoter. In some embodiments both circuits may be integrated into the bacterial chromosome. In some embodiments both circuits may be present on a plasmid. In some embodiments both circuits may be present on a plasmid. In some embodiments one circuit may be integrated into the bacterial chromosome and another circuit may be present on a plasmid.

In yet another embodiment, one or more strain(s) of genetically engineered bacteria expressing STING agonist producing circuitry, e.g., dacA, and one or more separate strain(s) genetically engineered bacteria expressing kynurenine consumption circuitry (e.g., kynureninase) may be administered sequentially, e.g., STING agonist producer (immune stimulator) may be administered before kynurenine consumer (immune sustainer). More generally, a bacterial strain expressing circuitry for immune initiation may be administered in conjunction with a separate bacterial strain expressing circuitry for immune sustenance, e.g., the immune initiator strain may be administered prior to the immune sustainer strain. For example, a bacterial strain expressing circuitry for immune initiation may be administered prior to a separate bacterial strain expressing circuitry for immune sustenance, e.g., the immune initiator strain. Alternatively, a bacterial strain expressing circuitry for immune initiation may be administered after a separate bacterial strain expressing circuitry for immune sustenance, e.g., the immune initiator strain. In yet another embodiment, a bacterial strain expressing circuitry for immune initiation may be administered concurrently with a separate bacterial strain expressing circuitry for immune sustenance, e.g., the immune initiator strain.

FIG. 15 depicts a schematic showing how genetically engineered bacteria of the disclosure can transform the tumor microenvironment by complementing stromal in immune deficiencies to achieve wide anti-tumor activity.

FIG. 16 depicts a schematic showing combinations of mechanisms for improved anti-tumor activity.

FIG. 17A and FIG. 17B depicts bar graphs showing production of cyclic-di-AMP (FIG. 17A) and consumption of kynurenine (FIG. 17B) for STING agonist producer SN3527, kynurenine consumer SYN2028, and combination strain (STING agonist producer plus kynurenine consumer) SYN3831.

FIG. 18A depicts a graph showing the growth (CFU per gram tumor tissue) of auxotrophic mutants ΔUraA, ΔThyA, and ΔDapA in CT26 Tumors over a 72 hour time period as indicated. FIG. 18B and FIG. 18C depicts graphs showing the growth (CFU per gram tumor tissue) of the auxotrophic mutant ΔThyA (SYN1605) compared to wildtype E. coli Nissle (SYN94) in B16F10 (FIG. 18B) and EL4 (FIG. 18C) tumors over a 72 hour time period as indicated.

FIG. 19A depicts a line graph of an in vivo analysis showing the effect of SYN4023 (comprising the tetracycline inducible Listeria monocytogenes dacA construct and ΔDapA mutation) on tumor growth (median tumor volume) over time at two different doses (1e7 and 1e8 CFUs) in the B16F10 model as compared to a saline control. FIG. 19B, FIG. 19C and FIG. 19D depict line graphs showing each individual mouse for the study shown in FIG. 19A.

FIG. 20A, and FIG. 20B depict graphs showing concentration of sepsis and cytokine storm related cytokines IL-1β (FIG. 20A) and TNF-a (FIG. 20B) in the blood of mice implanted with B16F10 tumors and subsequently treated with either 1e7 CFU SYN3527 (dacA, induced with tetracycline 4 hours post dose), 1e7 CFU SYN3527 (dacA, left uninduced), 1e8 CFU SYN4023 (dacA, and ΔDapA, induced), SYN94 (unmodified bacterium) or saline as control at various time points as indicated. LPS treatment was included as a positive control for sepsis. FIG. 20C and FIG. 20D depict graphs showing c-di-AMP concentrations (FIG. 20C) or CFU counts (FIG. 20D) in the tumor at various time points as indicated.

FIG. 21A depicts a line graph of an in vivo analysis showing the effect of SYN4023 (comprising the tetracycline inducible Listeria monocytogenes dacA construct and ΔDapA mutation) compared to saline injection control on tumor growth in the A20 tumor model (median tumor volume). FIG. 21B and FIG. 21C depict line graphs showing each individual mouse for the study shown in FIG. 21A.

FIG. 22A depicts a line graph of an in vivo analysis showing the effect of SYN4023 (DAP-STING, comprising the tetracycline inducible Listeria monocytogenes dacA construct and ΔDapA mutation) on tumor medians volumes over time, alone or in combination with an immune stimulator (agonistic anti-OX40, anti-41BB, or anti-GITR antibodies), in the B16F10 model as compared to controls or single agents alone (SYN4023, anti-ox40, anti-41BB, or anti-GITR antibodies plus saline). FIG. 22B, FIG. 22C, FIG. 22D, FIG. 22E, FIG. 22F, FIG. 22G, and FIG. 22H depict line graphs showing each individual mouse for the study shown in FIG. 22A.

FIG. 23A depicts a line graph showing that SYN4023 (comprising tet-inducible dacA and delta dapA) can elicit an abscopal effect in combination with intra-tumor injected anti-OX40 antibody in the A20 tumor model. Average median tumor volume is shown for each treatment group. Treated/Injected tumors are shown on the right of the graph while tumors receiving no treatment (Un-injected) are shown on the left. FIG. 23B and FIG. 23C depict line graphs showing the tumor volumes of the individual mice (naïve mice in FIG. 23B, and mice treated with SYN4023 in FIG. 23C) over time. FIG. 23D depicts a graph showing mouse survival over the duration of the study shown in FIG. 23A. FIG. 23E depicts a graph showing average mean bodyweight over duration of the study. FIG. 23F depicts a line graph showing the results of a re-challenge study, in which mice previously treated with SYN4023 (as shown in FIG. 23A-23E and having shown complete regression upon monitoring for at least 30 days) were implanted with A20 tumors in the left flank and CT26 tumors in the right flank as compared to naïve age-matched mice implanted with the same tumors. Average median tumor volume is shown for each treatment group. FIG. 23G and FIG. 23H depict line graphs showing the tumor volumes of the individual mice from the study shown in FIG. 23F over time (naïve mice in FIG. 23G and mice previously treated with SYN4023 in FIG. 2311). FIG. 23I depicts a graph showing the entire 2-part study querying abcopal effect and immunological memory potential (rechallenge with A20 is depicted). The graph shows individual tumor measurements for the indicated experimental groups.

FIG. 24 and depicts bar graphs showing in vivo analysis of GFP expression levels achieved with ATC, aspirin, cumate, and low oxygen (FNR) inducible promoters in the B16 tumor model in the presence or absence of the inducer at 1 and 16 hours as indicated. The percentage of induced (GFP+) bacteria among all bacteria recovered (RFP+).

FIG. 25 shows the level of gene expression as measured by geometric mean fluorescence intensity (MFI) for GFP+/RFP+ bacteria for the analysis described in FIG. 24.

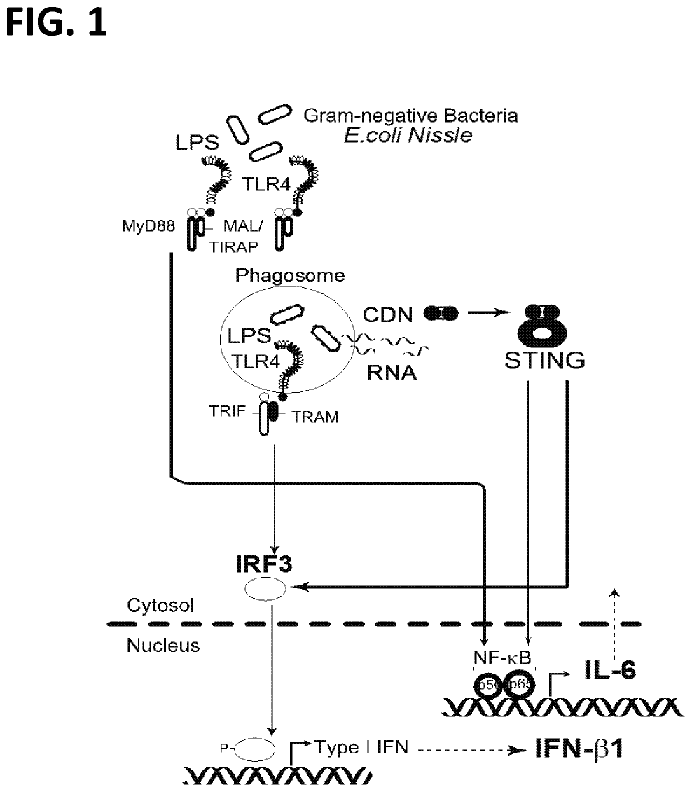

FIG. 26A, FIG. 26B, FIG. 26C, and FIG. 26D depict line graphs of individual mice in an in vivo analysis showing the effect of the STING agonist producing strain SYN4449 on B16-F10 tumor volume over time at three different doses (1e7 (FIG. 26B), 1e8 (FIG. 26C) and 1e9 (FIG. 26D)) and indicate that administration of SYN4449 at a dose of leg results in rejection or control of tumor growth over this time period in the B16.F10 tumor model. FIG. 26A depicts a line graph of individual mice treated with a saline control.

FIG. 27A, FIG. 27B, and FIG. 27C depict line graphs of individual mice in an in vivo analysis showing the effect of the STING agonist producing strain SYN4449 on tumor volume over time at three different doses (1e6, 1e7 and 1e8) and demonstrates that SYN4449 (comprising plasmid based FNR-dacA and delta dapA) drives dose-dependent tumor control in A20 lymphoma model. CR=complete response. FIG. 27D depicts a line graph of individual mice treated with a saline control.

FIG. 28A depicts a bar graph showing SYN4449 comprising a dapA mutation and FNR-dacA on a plasmid (ΔDAP, 15A-fnr-dacA) as compared to SYN94 (streptomycin resistant Nissle), demonstrating that SYN4449 produces c-di-AMP. FIG. 28B and FIG. 28C depict bar graphs showing in vitro c-diAMP production of SYN4910 (FIG. 28B) and SYN4939 (FIG. 28C) as compared to SYN94. FIG. 28D depicts a bar graph showing a comparison of in vitro Kynurenine consumption of SYN2306, SYN4939 and SYN94 at 0, 2, and 4 hours. SYN4910 comprises a phage deletion, a DAPA auxotrophy, a ThyA auxotrophy, and FNR-DacA integrated at the HA9/10 site (ΔΦ, ΔDAP, ΔThyA, HA9/10::fnr-DacA). SYN4939, a c-diAMP producing and kynurenine consuming combination strain, comprises chromosomally integrated, kynureninase under control of a constitutive promoter, a deletion in TrpE, a phage deletion, a DapA auxotrophy and a ThyA auxotrophy, and FNR-DacA integrated at the HA9/10 site (PSynJ23119-pKYNase, ΔTrpE, ΔΦ, ΔDAP, ΔThyA, HA9/10::fnr-DacA). SYN2306 comprises a constitutively expressed kynureninase (Pseudomonas fluorescens) and a deletion in TrpE (HA3/4::PSynJ23119-pKYNase delta TrpE). SYN94 control: streptomycin resistant Nissle.

FIG. 29A and FIG. 29B depict bar graphs showing a comparison of in vitro c-diAMP production by SYN4739 (FIG. 29A) or SYN4939 (FIG. 29B, with SYN94 (streptomycin resistance Nissle). FIG. 29C and FIG. 29D depict bar graphs showing a comparison of in vitro kynurenine consumption at 0, 2, and 4 hours by SYN2028 and SYN4739 (FIG. 29C) or SYN2306 and SYN4939 (FIG. 29D) with SYN94. SYN4739 comprises a constitutively expressed kynureninase from Pseudomonas fluorescens, a deletion in TrpE, and a ThyA auxotrophy (HA3/4::PSynJ23119-pKYNase, ΔTrpE, ΔThyA, HA9/10::fnr-DacA). SYN4939, a c-diAMP producing and kynurenine consuming combination strain, comprises chromosomally integrated, kynureninase under control of a constitutive promoter, a deletion in TrpE, a phage deletion, a DAPA auxotrophy and a ThyA auxotrophy, and FNR-DacA integrated at the HA9/10 site (PSynJ23119-pKYNase, ΔTrpE, ΔΦ, ΔDAP, ΔThyA, HA9/10::fnr-DacA). SYN2028 comprises chromosomally integrated kynureninase from Pseudomonas fluorescence under control of a constitutive promoter and a deletion in TrpE (HA3/4::PSynJ23119-pKYNase delta TrpE). SYN2306 comprises a constitutively expressed kynureninase (Pseudomonas fluorescens) and a deletion in TrpE (HA3/4::PSynJ23119-pKYNase delta TrpE). SYN94: streptomycin resistant Nissle.

FIG. 30 and FIG. 31 depict bar graphs showing a comparison of in vitro c-diAMP production and in vitro kynurenine consumption at 0, 2, and 4 hours between SYN2306, SYN4789, SYN4939, and SYN94. SYN2306 comprises a constitutively expressed kynureninase (Pseudomonas fluorescens) and a deletion in TrpE (HA3/4::PSynJ23119-pKYNase delta TrpE). SYN94: streptomycin resistance Nissle. SYN4789 comprises a constitutively expressed kynureninase from Pseudomonas fluorescens, a deletion in TrpE, and a ThyA auxotrophy (HA3/4::PSynJ23119-pKYNase, ΔTrpE, ΔThyA, HA9/10::fnr-DacA). SYN4939, a c-diAMP producing and kynurenine consuming combination strain, comprises chromosomally integrated, kynureninase under control of a constitutive promoter, a deletion in TrpE, a phage deletion, a DAPA auxotrophy and a ThyA auxotrophy, and FNR-DacA integrated at the HA9/10 site (PSynJ23119-pKYNase, ΔTrpE, ΔΦ, ΔDAP, ΔThyA, HA9/10::fnr-DacA). SYN94: streptomycin resistant Nissle.

FIG. 32 depicts a line graph of an in vitro analysis of the activity of the STING agonist producing strain SYN4737 on IFN-beta1 induction in RAW 264.7 cells at various multiplicities of infection (MOI) at 4 hours demonstrates that SYN4737 (comprising a phage deletion, a DAPA auxotrophy, and FNR-DacA integrated at the HA9/10 site (ΔΦ, ΔDAP, HA9/10::fnr-DacA)) drives dose-dependent IFN-beta1 induction in RAW 264.7 cells (immortalized murine macrophage cell line). Briefly, bacteria (WT Nissle (Labeled in graph as “SYN”) or SYN4737 were pre-induced for 4 hours in an anaerobic chamber to induce STING agonist synthesis and then were co-cultured at various multiplicities of infection (MOI) with 0.5×106 RAW 264.7 cells for 4 hours and protein present in RAW 264.7 cell supernatant were analyzed.

FIG. 33A and FIG. 33B, depict graphs showing in vitro production c-di-AMP and bacterial cGAMP, of various strains comprising cGAS orthologs (putative cGAMP synthases).

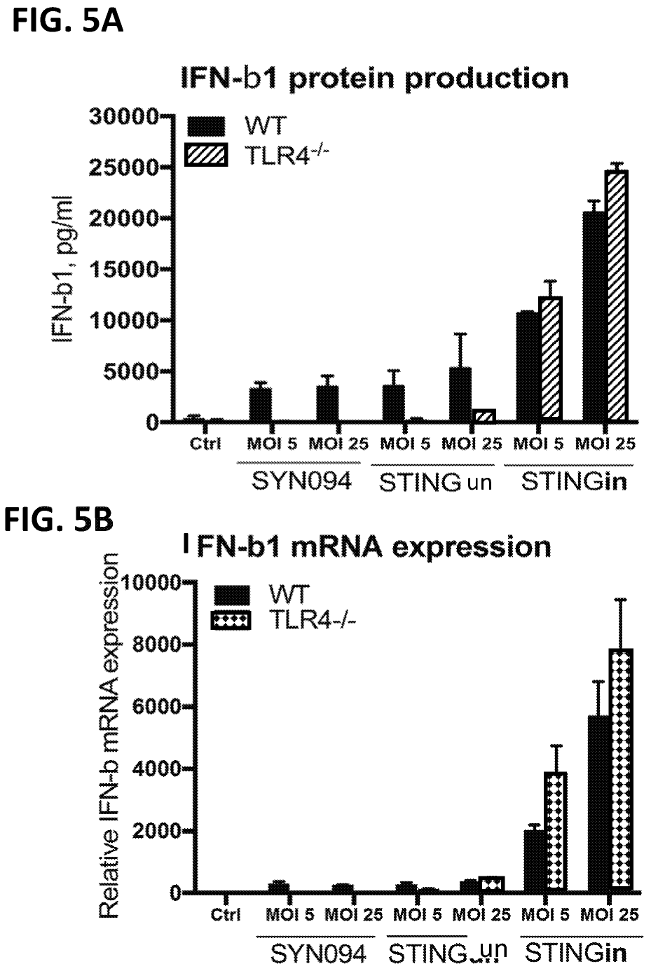

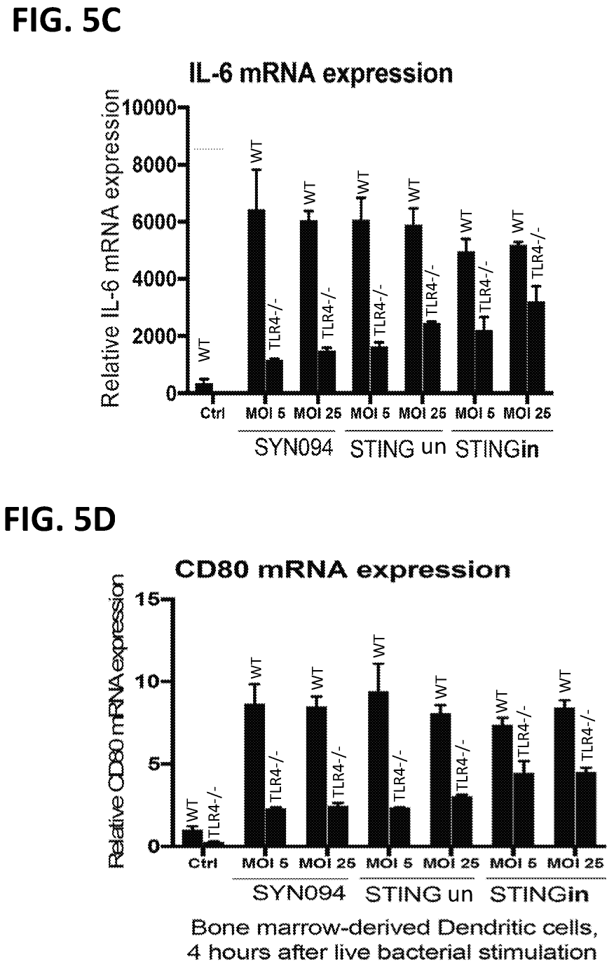

FIG. 34A and FIG. 34B depict bar graphs showing the ability of the E. coli Nissle strains SYN3529 (Nissle p15A Ptet-CodA) and SYN3620 (Nissle p15A Ptet-CodA::Upp fusion) to convert 5-FC to 5-FU. The graphs show 5-FC levels (FIG. 34A) and 5-FU levels (FIG. 34B) after an assay time of 2 hours.

FIG. 35A depicts a schematic showing an outline of an in vivo mouse study, the results of which are shown in FIG. 35B, FIG. 35C, FIG. 35D, and FIG. 35E. FIG. 35B depicts a line graph showing the average mean tumor volume of mice implanted with B16-F10 tumors and treated with PBS, SYN3620 (comprising pUC-Kan-tet-CodA::Upp fusion) or SYN3529 (comprising pUC-Kan-tet-CodA (cytosine deaminase)). FIG. 35C depicts line graphs showing tumor volume of individual mice in the study. FIG. 35D depicts a graph showing the tumor weight at day 6. FIG. 35E depicts a graph showing intratumoral concentration of 5-FC at day 6 measured via mass spectrometry.

FIG. 36A depicts a schematic showing an outline of an in vivo mouse study, the results of which are shown in FIGS. 36B and 36C. FIG. 36B depicts graphs showing bacterial colonization of tumors as measured by colony forming units (CFU). FIG. 36C depicts graphs showing the relative expression of CCR7 (left) or CD40 (right) as measured by median Mean Fluorescence Intensity (MFI) on the indicated immune cell populations for intratumoral lymphocytes isolated from CT26 tumors on day 8 measured via flow cytometry.

FIG. 37 depicts a graph showing results of a cell based assay showing IkappaBalpha degradation in HeLa cells upon treatment with supernatants of the TNFα secreter SYN2304 (PAL::Cm p15a TetR Ptet-phoA TNFa), the parental control SYN1557, and a recombinant IL-15 control.

FIG. 38A depicts a schematic showing an outline of an in vivo mouse study, the results of which are shown in FIG. 38B-38D. FIG. 38B depicts graphs showing bacterial colonization of tumors as measured by colony forming units (CFU). FIG. 38C depicts graphs showing the relative concentration of TNFα in CT26 tumors as measured by ELISA. FIG. 38D depicts a line graph showing the average mean tumor volume of mice implanted with CT26 tumors and treated with SYN (DOM Mutant) or SYN-TNFα (comprising PAL::CM p15a TetR Ptet-PhoA-TNFα).

FIG. 39A and FIG. 39B depict graphs showing results of a cell based assay showing STAT1 phosphorylation in mouse RAW264.7 cells upon treatment with supernatants of the IFNgamma secreter SYN3543 (PAL::Cm p15a Ptet-87K PhoA-mIFNg), the parental control SYN1557, and a recombinant IL-15 control.

FIG. 40A depicts a schematic showing an outline of an in vivo mouse study, the results of which are shown in FIGS. 40B and 40C. FIG. 40B depicts graphs showing bacterial colonization of tumors as measured by colony forming units (CFU). FIG. 40C depicts graphs showing the relative concentration of IFNγ in CT26 tumors as measured by ELISA.

FIG. 41 depicts a bar graph of in vitro arginine levels produced by streptomycin-resistant Nissle (SYN-UCD103), SYN-UCD205, and SYN-UCD204 under inducing (+ATC) and non-inducing (−ATC) conditions, in the presence (+O₂) or absence (−O₂) of oxygen. SYN-UCD103 is a control Nissle construct. SYN-UCD205 comprises ΔArgR and argA^{fbr} expressed under the control of a FNR-inducible promoter on a low-copy plasmid. SYN-UCD204 comprises ΔArgR and argA^{fbr} expressed under the control of a tetracycline-inducible promoter on a low-copy plasmid.

FIG. 42A and FIG. 42B depict bar graphs of ammonia levels in the media at various time points post anaerobic induction. FIG. 42A depicts a bar graph of the levels of arginine production of SYN-UCD205, SYN-UCD206, and SYN-UCD301 measured at 0, 30, 60, and 120 minutes. FIG. 42B depicts a bar graph of the levels of arginine production of SYN-UCD204 (comprising ΔArgR, PfnrS-ArgAfbr on a low-copy plasmid and wild type ThyA), SYN-UCD301, SYN-UCD302, and SYN-UCD303 (all three of which comprise an integrated FNR-ArgAfbr construct; SYN-UCD301 comprises ΔArgR, and wtThyA; SYN-302 and SYN-UCD303 both comprise ΔArgR, and ΔThyA, with chloramphenicol or kanamycin resistance, respectively). Results indicate that chromosomal integration of FNR ArgA fbr results in similar levels of arginine production as seen with the low copy plasmid strains expressing the same construct.

FIG. 43 depicts a line graph showing the in vitro efficacy (arginine production from ammonia) in an engineered bacterial strain harboring a chromosomal insertion of ArgAfbr driven by an fnr inducible promoter at the malEK locus, with ΔArgR and ΔThyA and no antibiotic resistance was assessed (SYN-UCD303). Streptomycin resistant E. coli Nissle (Nissle) is used as a reference.

FIG. 44A depicts a chart showing the administration schema for the study shown in 40A, 40B, 40C, 44E, and 44F. FIGS. 44B, 44C, 44D, 44E, and 44F depict a line graphs for each individual mouse of an in vivo analysis of the effect on tumor volume of a combination treatment with the chemotherapeutic agent cyclophosphamide (nonmyeloablative chemotherapy, preconditioning) and an arginine producing strain (SYN-UCD304; integrated FNR-ArgAfbr construct; ΔArgR, FIG. 44E) or kynurenine consuming strain (SYN2028, FIG. 44F). The effect of the combination treatment was compared to treatment with vehicle alone (FIG. 44B), cyclophosphamide alone (FIG. 44C), or SYN94 (streptomycin resistant wild type Nissle, FIG. 44D). The data suggest anti-tumor activity of the arginine producing and the kynurenine-consuming strains in combination with cyclophosphamide. In this study, BALB/c mice were implanted with CT26 tumors; cyclophosphamide (CP) was administered IP at 100 mg/kg; bacteria were administered intratumorally at 1×10^{e7} (in a 100 ul volume). The administration schema is shown in FIG. 44A.

FIG. 45A and FIG. 45B depicts the results of a human T cell transwell assay where the number of migratory cells was measured via flow cytometry following addition of SYN-CXCL10 supernatants diluted at various concentrations in SYN bacterial supernatant. Anti-CXCR3 was added to control wells containing 100% SYN-CXCL10 supernatant to validate specificity of the migration for the CXCL10-CXCR3 pathway. FIG. 45A depicts the total number of migrated cells. FIG. 45B depicts the Migration relative to no cytokine control.

FIG. 46. depicts a line graph showing the results of a cell-based assay showing STATS phosphorylation in CD3+IL15RAalpha+ T-cells upon treatment with supernatants of the IL-15 secreter SYN3525 (PAL::Cm p15a Ptet-PpiA (ECOLIN_18620)-IL-15-Sushi), the parental control SYN1557, and a recombinant IL-15 control.

FIG. 47 depicts a bar graph showing that strains SYN1565 (comprising PfnrS-nupC), SYN1584 (comprising PfnrS-nupC; PfnrS-xdhABC) SYN1655 (comprising PfnrS-nupC; PfnrS-add-xapA-deoD) and SYN1656 (comprising PfnrS-nupC; PfnrS-xdhABC; PfnrS-add-xapA-deoD) can degrade adenosine in vitro, even when glucose is present.

FIG. 48 depicts a bar graph showing adenosine degradation at substrate limiting conditions, in the presence of 1 uM adenosine, which corresponds to adenosine levels expected in the in vivo tumor environment. The results show that a low concentration of activated SYN1656 (1e6 cells), (and also other strains depicted), are capable of degrading adenosine below the limit of quantitation.

FIG. 49 depicts a line graph of an in vivo analysis of the effect of adenosine consumption by engineered E. coli Nissle (SYN1656), alone or in combination with anti-PD1, on tumor volume. The data suggest anti-tumor activity of adenosine-consuming strain as single agent and in combination with aPD-1.

FIG. 50A and FIG. 50B depict graphs showing that combination of adenosine consuming strain SYN1656 (SYN-Ade) with an anti-PD-1/anti-CTLA4 cocktail elicits high numbers of tumor rejections. To investigate the anti-tumor activity of SYN1656 in combination with anti-PD-1/anti-CTLA4 checkpoint inhibition, MC38 tumors were established in C57BL6 mice. When tumors were 60-80 mm3 in size, animals were treated bi-weekly intra-tumorally with saline control, intraperitoneally with a cocktail of anti-PD-1 and anti-CTLA4 antibodies (10 and 5 mg/kg, respectively), or with a combination of unmodified bacteria (SYN) or SYN1656 (SYN-Ade) and anti-PD-1/anti-CTLA4, and tumor volumes were assessed twice a week. FIG. 50A depicts the median tumor volume and FIG. 50B depicts the percentage of animals remaining on study over time using <2000 mm3 as a survival surrogate; FIG. 50C, FIG. 50D, FIG. 50E, and FIG. 50F depict graphs showing tumor volumes for individual animals from each treatment group.

FIG. 51 depicts a bar graph showing the kynurenine consumption rates of original and ALE evolved kynureninase expressing strains in M9 media supplemented with 75 uM kynurenine. Strains are labeled as follows: SYN1404: E. coli Nissle comprising a deletion in Trp:E and a medium copy plasmid expressing kynureninase from Pseudomonas fluorescens under the control of a tetracycline inducible promoter (Nissle deltaTrpE::CmR+Ptet-Pseudomonas KYNU p15a KanR); SYN2027: E. coli Nissle comprising a deletion in Trp:E and expressing kynureninase from Pseudomonas fluorescens under the control of a constitutive promoter (the endogenous 1pp promoter) integrated into the genome at the HA3/4 site (HA3/4::Plpp-pKYNase KanR TrpE::CmR); SYN2028: E. coli Nissle comprising a deletion in Trp:E and expressing kynureninase from Pseudomonas fluorescens under the control of a constitutive promoter (the synthetic J23119 promoter) integrated into the genome at the HA3/4 site (HA3/4::PSynJ23119-pKYNase KanR TrpE::CmR); SYN2027-R1: a first evolved strain resulting from ALE, derived from the parental SYN2027 strain (Plpp-pKYNase KanR TrpE::CmR EVOLVED STRAIN Replicate 1). SYN2027-R2: a second evolved strain resulting from ALE, derived from the parental SYN2027 strain (Plpp-pKYNase KanR TrpE::CmR EVOLVED STRAIN Replicate 2). SYN2028-R1: a first evolved strain resulting from ALE, derived from the parental SYN2028 strain (HA3/4::PSynJ23119-pKYNase KanR TrpE::CmR EVOLVED STRAIN Replicate 1). SYN2028-R2: a second evolved strain resulting from ALE, derived from the parental SYN2028 strain (HA3/4::PSynJ23119-pKYNase KanR TrpE::CmR EVOLVED STRAIN Replicate 1).

FIG. 52A and FIG. 52B depict dot plots showing intratumoral kynurenine depletion by strains producing kynureninase from Pseudomonas fluorescens. FIG. 52A depicts a dot plot showing a intra tumor concentrations observed for the kynurenine consuming strain SYN1704, carrying a constitutively expressed Pseudomonase fluorescens kynureninase on a medium copy plasmid. FIG. 52B. depicts a dot plot showing a intra tumor concentrations observed for the kynurenine consuming strain SYN2028 carrying a constitutively expressed chromosomally integrated copy of Pseudomonase fluorescens kynureninase. The IDO inhibitor INCB024360 is used as a positive control.

FIG. 53A and FIG. 53B, depict dot plots showing concentrations of intratumoral kynurenine (FIG. 53A) and plasma kynurenine (FIG. 53B) measured in mice implanted with CT26 tumors administered either saline, or SYN1704. A significant reduction in intratumoral (P<0.001) and plasma (P<0.005) concentration of kynurenine was observed for the kynurenine consuming strain SYN1704 compared to saline control. Tryptophan levels remained constant (data not shown).

FIGS. 54A, 54B, and 54C depict graphs showing the effects of single administration of a KYN-consuming strain in CT26 tumors has on tumoral KYN levels in the tumor (FIG. 54A) and plasma (FIG. 54B), and tumor weight (FIG. 54C). Mice were dosed with SYN94 or SYN1704 at the 1e8 CFU/mL via intratumoral dosing. Animals were sacrificed and blood and tissue was collected at the indicated times.

FIG. 55 depicts a Western blot analysis of bacterial supernatants showing murine CD40L1 (47-260) and CD40L2 (112-260) secreted by E. coli strains SYN3366 and SYN3367 are detected by a mCD40 antibody.

FIG. 56 depicts a line graph of an in vivo analysis of the effect of kynurenine consumption by kynurenine consuming strain SYN2028 carrying a constitutively expressed chromosomally integrated copy of Pseudomonas fluorescens kynureninase), alone or in combination with anti-CTLA4 antibody, compared to vehicle or anti-CTLA-4 antibody alone, on tumor volume. The data suggest anti-tumor activity of the kynurenine-consuming strain as single agent and in combination with anti-CTLA4 antibody, and that SYN2028 improves αCTL-4-mediated anti-tumor activity in CT26. In this study, BALB/c mice were implanted with CT26 tumors; anti-CTLA4 antibody was administered IP at 100 ug/mouse; Bacteria were administered intratumorally at 1×10^{e7}; bacteria and antibodies were all administered biweekly.

FIGS. 57A, 57B, 57C, and 57D depict line graphs showing each individual mouse for the study shown in FIG. 56. FIG. 57E depicts the corresponding Kaplan-Meier plot.

FIG. 58A, FIG. 58B, FIG. 58C, FIG. 58D, FIG. 58E depicts a line graphs showing that Kyn consumer SYN2028 in combination with αντιCTL-4 and anti-PD1 antibodies has improved anti-tumor activity in MC38 tumors. FIGS. 58B, 58C, 58D, and 58E depict line graphs showing each individual mouse for the study shown in FIG. 58A. Kyn consumer SYN2028 in combination with anti-CTL-4 and anti-PD1 antibodies has improved anti-tumor activity in MC38 tumors (FIG. 58E) over vehicle (FIG. 58B), anti-CTLA4 and anti-PD1 antibodies alone (FIG. 58C), or SYN94 (streptomycin resistant E. coli Nissle) plus anti-CTLA4 and anti-PD1 antibodies (FIG. 58D); i.e., the kynurenine consumer has the ability to improve anti-CTLA-4/anti-PD1 antibody-mediated anti-tumor activity. FIG. 58F depicts the corresponding Kaplan-Meier plot.

FIG. 59A and FIG. 59B depict an analysis of tumor colonization and in vivo activity of the kynurenine consuming strain SYN2028 (SYN-Kyn) in the B16F10 tumor model. Upon reaching a tumor size of −40-80 mm3, mice received 1e6 CFUs of unmodified (SYN-WT) or SYN2028 (SYN-Kyn) via intratumoral injection. At 24 and 72 hours post-injection, tumors were homogenized and colony forming units (CFU) were determined by plating on LB antibiotic selective plates (FIG. 59A) or kynurenine levels were determined by LCMS (FIG. 59B).

FIG. 60A and FIG. 60B depict graphs showing that SYN1565 (SYN-Ade) and SYN2028 (SYN-Kyn) demonstrate robust tumor colonization after intra-tumoral administration. To assess the ability of the adenosine-consuming strain SYN1565 or kynurenine-consuming strain SYN2028 to colonize tumors, B16.F10 tumors were established in C57BL6 mice. When tumors reached 100-150 mm3 in size, SYN1565, SYN2028 (1e6 cells/dose) or saline control were administered intra-tumorally as a single injection. Colony forming units (CFU) per gram of tumor tissue were calculated 7 days post injection and results are shown in FIG. 60A. For comparison, CFU per gram of tumor tissue of the unmodified Nissle chassis (SYN) 7 days post a single 1e6 cell/dose injection is included (FIG. 60B).

FIG. 61 depicts a Western Blot analysis of total cytosolic extracts of a wild type E. coli (lane 1) and of a strain expressing anti-PD1 scFv (lane 2).

FIG. 62 depicts a diagram of a flow cytometric analysis of PD1 expressing EL4 cells which were incubated with extracts from a strain expressing tet inducible anti-PD1-scFv, and showing that anti-PD1-scFv expressed in E. coli binds to PD1 on mouse EL4 cells.

FIG. 63 depicts a Western Blot analysis of total cytosolic extracts of various strain secreting anti-PD1 scFv. A single band was detected around 34 kDa in lane 1-6 corresponding to extracts from SYN2767, SYN2769, SYN2771, SYN2773, SYN2775 and SYN2777, respectively.

FIG. 64 depicts a diagram of a flow cytometric analysis of PD1 expressing EL4 cells, which were incubated with extracts from a E. coli Nissle strain secreting tet-inducible anti-PD1-scFv, showing that anti-PD1-scFv secreted from E. coli Nissle binds to PD1 on mouse EL4 cells.

FIG. 65 depicts a diagram of a flow cytometric analysis of PD1 expressing EL4 cells, which were incubated with various amounts of extracts (0, 2, 5, and 15 ul) from an E. coli Nissle strain secreting tet-inducible anti-PD1-scFv, showing that anti-PD1-scFv secreted from E. coli Nissle binds to PD1 on mouse EL4 cells, in a dose dependent manner.

FIG. 66A and FIG. 66B depicts diagrams of a flow cytometric analysis of EL4 cells. FIG. 66A depicts a competition assay, in which extracts from a E. coli Nissle strain secreting tet-inducible anti-PD1-scFv was incubated with various amounts of soluble PDL1 (0, 5, 10, and 30 ug) showing that PDL1 can dose-dependently compete with the binding of anti-PD1-scFv secreted from E. coli Nissle to PD1 on mouse EL4 cells. FIG. 66B shows the IgG control.

FIG. 67 depicts a Western blot analysis of bacterial supernatants from SYN2996 (lane 1), SYN3159 (lane 2), SYN3160 (lane 3), SYN3021 (lane 4), SYN3020 (lane 5), and SYN3161 (lane 6) showing that WT mSIRPα, mCV1SIRPα, mFD6×2SIRPα, mCV1SIRPα-IgG4, mFD6SIRPα-IgG4, and anti-mCD47 scFv are secreted from these strains, respectively.

FIG. 68 depicts a diagram of a flow cytometric analysis of CD47 expressing CT26 cells which were incubated with supernatants from a SYN1557 (1; ΔPAL parental strain), SYN2996 (2; expressing tet inducible mSIRPα), SYN3021 (3; expressing tet inducible anti-mCD47scFv), SYN3161 (4; expressing tet inducible mCV1SIRPα-hIgG fusion) and showing that secreted products expressed in E. coli can bind to CD47 on mouse CT26 cells.

FIG. 69 depicts a diagram of a flow cytometric analysis of CD47 expressing CT26 cells which were incubated with supernatants from a SYN1557 (1; ΔPAL parental strain), SYN3020 (2; expressing tet inducible mFD6SIRPα-hIgG fusion), SYN3160 (3; expressing tet inducible FD1×2SIRPα), SYN3159 (4; expressing tet inducible mCV1SIRPα), SYN3021 (5; expressing tet inducible mCV1SIRPα-hIgG fusion) and showing that secreted products expressed in E. coli can bind to CD47 on mouse CT26 cells.

FIG. 70 depicts a diagram of a flow cytometric analysis of CT26 cells. A competition assay was conducted, in which extracts from a E. coli Nissle strain secreting tet-inducible murine SIRPα was incubated with recombinant SIRPα showing that recombinant SIRPα can compete with the binding of SIRPα secreted from E. coli Nissle to CD47 on CT26 cells.

FIG. 71 depicts a diagram of a flow cytometric analysis of CT26 cells. A competition assay was conducted, in which extracts from a E. coli Nissle strain secreting tet-inducible murine SIRPα was incubated with an anti-CD47 antibody showing that the antibody can compete with the binding of SIRPα secreted from E. coli Nissle to CD47 on CT26 cells.

FIG. 72 depicts a Western blot analysis of bacterial supernatants from SYN2997 (lane 1) and SYN2998 (lane 2), showing that mouse and human hyaluronidases are secreted from these strains, respectively.

FIG. 73 depicts a bar graph showing hyaluronidase activity of SYN1557 (parental strain ΔPAL), SYN2997 and SYN2998 as a measure of hyaluronan degradation in an ELISA assay.

FIG. 74A depicts a Western blot analysis of bacterial supernatants from SYN3369 expressing tetracycline inducible leech hyaluronidase (lane 1) and SYN1557 (parental strain ΔPAL) (lane 2), showing that leech hyaluronidase is secreted from SYN3369. M=Marker. FIG. 74B and FIG. 74C depict a bar graphs showing hyaluronidase activity as a measure of hyaluronan degradation in an ELISA assay.

FIG. 74B shows a positive control with recombinant hyaluronidase. FIG. 74C shows hyaluronidase activity of SYN1557 (parental strain ΔPAL), and SYN3369 expressing tetracycline inducible leech hyaluronidase.

FIG. 75 depicts a map of exemplary integration sites within the E. coli 1917 Nissle chromosome. These sites indicate regions where circuit components may be inserted into the chromosome without interfering with essential gene expression. Backslashes (/) are used to show that the insertion will occur between divergently or convergently expressed genes. Insertions within biosynthetic genes, such as thyA, can be useful for creating nutrient auxotrophies. In some embodiments, an individual circuit component is inserted into more than one of the indicated sites. In some embodiments, multiple different circuits are inserted into more than one of the indicated sites. Accordingly, by inserting circuitry not multiple sites into the E. coli 1917 Nissle chromosome a genetically engineered bacterium may comprise circuitry allowing multiple mechanisms of action (MoAs).

FIG. 76 depicts a graph showing CFU of bacteria detected in the tumor at various time points post intratumoral (IT) dose with 100 ul SYN94 (streptomycin resistant Nissle) or SYN1557 (Nissle ΔPAL::CmR) (1e7 cells/dose). No bacteria were detected in the blood at these time points.

FIG. 77 depicts a graph showing CFU of bacteria detected in the tumor (CT26 at various time points post intratumoral (IT) dose with 100 ul SYN94 (streptomycin resistant Nissle) at 1e7 and 1e8 cells/dose. Bacterial counts in the tumor tissue were similar at both doses.

FIG. 78A and FIG. 78B depict graphs showing bacterial concentrations detected in various tissues (FIG. 78A) and TNFa levels measured in serum, tumor and liver (FIG. 78B) at 48 hours post intratumor administration 10⁷ CFU/dose SYN94 (streptomycin resistant Nissle) or saline administration and in naïve animals. Bacteria were predominantly present in the tumor and absent in other tissues tested. TNFa levels measured were similar in all serum, tumor and liver between SYN94, Saline treated and naïve groups.

FIG. 79 depict graphs showing high levels of c-diAMP production are achieved in vivo through anaerobic induction using a low oxygen promoter (FNR promoter) to drive expression of DacA (plasmid based FNR-DacA, ΔDAP). B16 cells were implanted at 2e5; and at day 14 post implant, when tumors reached about ˜250-400 mm3, mice were divided into three experimental groups. Group 1 was injected once with PBS (n=1); Group 2 (n=3) was injected with SYN766 (DAP-WT; leg cells). Group 3 (n=3) was injected with SYN4449 (plasmid based FNR-DacA, ΔDAP; 1e9 cells); At 24 hours post dose, tumors were extracted, and c-di-AMP production was measured by LC-MS/MS.

FIG. 80 depicts graphs showing high levels of c-diAMP production are achieved in vivo through anaerobic induction using a low oxygen promoter (FNR promoter) to drive the expression of an integrated DacA. B16 cells were implanted at 2e5; and at day 14 post implant, when tumors reached about ˜250-400 mm3, mice were divided into 2 experimental groups. Group 1 was injected once with PBS (n=3); Group 2 (n=3) was injected with SYN4910 (DAP-FNR-STING integrated further comprising ΔThyA and ΔDapA auxotrophy and phage deletion; 1e9 cells); At 24 hours post dose, tumors were extracted, and c-di-AMP production was measured by LC-MS/MS.

FIGS. 81A, 81B, 81C, and 81D depict graphs showing efficacy of SYN4910 (DAP-FNR-STING) integrated further comprising ΔThyA and ΔDapA auxotrophy and phage deletion) in the B16 model. Briefly, B16 cells were implanted as described above. Tumor growth was monitored until the tumors reached ˜100 mm\hat{ }3. On day 0, mice were randomized into groups (N=10 per group) for intratumor dosing as follows: PBS (group 1, vehicle control), SYN4740 (ΔThyA, ΔDapA, ΔΦ; group 2, 1e9 CFU,), and SYN4910 (group 3, 1e9 CFU). Tumor sizes were measured and mice were injected I.T. with bacteria or PBS on day 0, 2, and 5. Tumor volumes were recorded two times in a week. Results indicate that administration of SYN4910 drives tumor control and rejection in B16 tumor lymphoma model.

FIG. 82 depicts a graph showing production of the human cyclic GAMP (2′3′-cGAMP) analog, via the expression of human cyclic GAMP synthase (hcGAS). The genetic circuit for hcGAS comprises a p15a origin plasmid and a tetracycline-inducible promoter (Ptet) driving the expression of the coding sequence for the hcGAS protein that was codon-optimized for expression in E. coli. As indicated, a strain was generated as follow (1) strain which comprises the plasmid alone; (2) strain which comprises the p15-ptet-hcGAS and a dapA auxotrophic modification (3) strain which comprises the p15-ptet-hcGAS and a kynurenine consumption circuit (chromosomally integrated kynureninase under control of a constitutive promoter); (4) strain which comprises the p15-ptet-hcGAS and chromosomally integrated kynureninase under control of a constitutive promoter, and an arginine production circuit comprising feedback resistant ArgA under control of the low oxygen inducible FNR promoter, and a deletion in the endogenous or native argR gene. To produce the 2′3′-cGAMP analog, overnight cultures and control strains were grown in LB containing appropriate antibiotic. These were back diluted into M9 minimal media containing 0.5% glucose and appropriate antibiotics. These were grown for two hours before induction with 500 ng/mL of anhydrotetracycline (ATC), then subsequently allowed to incubate a further 2 hours. 1 mL of the culture was removed, centrifuged at 8000×g for 5 minutes and the supernatant discarded. These pellets were then used in quantify the intracellular concentrations of the 2′3′-cGAMP STING agonist by LC/MS.

### DESCRIPTION OF THE EMBODIMENTS

Certain tumors are particularly difficult to manage using conventional therapies. Hypoxia is a characteristic feature of solid tumors, wherein cancerous cells are present at very low oxygen concentrations. Regions of hypoxia often surround necrotic tissues and develop as solid forms of cancer outgrow their vasculature. When the vascular supply is unable to meet the metabolic demands of the tumor, the tumor's microenvironment becomes oxygen deficient. Multiple areas within tumors contain <1% oxygen, compared to 3-15% oxygen in normal tissues (Vaupel and Hockel, 1995), and avascular regions may constitute 25-75% of the tumor mass (Dang et al., 2001). Approximately 95% of tumors are hypoxic to some degree (Huang et al., 2004). Systemically delivered anticancer agents rely on tumor vasculature for delivery, however, poor vascularization impedes the oxygen supply to rapidly dividing cells, rendering them less sensitive to therapeutics targeting cellular proliferation in poorly vascularized, hypoxic tumor regions. Radiotherapy fails to kill hypoxic cells because oxygen is a required effector of radiation-induced cell death. Hypoxic cells are up to three times more resistant to radiation therapy than cells with normal oxygen levels (Bettegowda et al., 2003; Tiecher, 1995; Wachsberger et al., 2003). For all of these reasons, nonresectable, locally advanced tumors are particularly difficult to manage using conventional therapies.

In addition to the challenges associated with targeting a hypoxic environment, therapies that specifically target and destroy cancers must recognize differences between normal and malignant tissues, including genetic alterations and pathophysiological changes that lead to heterogeneous masses with areas of hypoxia and necrosis.

The disclosure relates to genetically engineered microorganisms, e.g., genetically engineered bacteria, pharmaceutical compositions thereof, and methods of modulating or treating cancer. In certain embodiments, the genetically engineered bacteria are capable of targeting cancerous cells. In certain embodiments, the genetically engineered bacteria are capable of targeting cancerous cells, particularly in low-oxygen conditions, such as in hypoxic tumor environments. In certain embodiments, the genetically engineered bacteria are delivered locally to the tumor cells. In certain aspects, the compositions and methods disclosed herein may be used to deliver one or more immune modulators to cancerous cells or produce one or more immune modulators in cancerous cells.

This disclosure relates to compositions and therapeutic methods for the local and tumor-specific delivery of immune modulators in order to treat cancers. In certain aspects, the disclosure relates to genetically engineered microorganisms that are capable of targeting cancerous cells and producing one or more effector molecules e.g., immune modulators, such as any of the effector molecules provided herein. In certain aspects, the disclosure relates to genetically engineered bacteria that are capable of targeting cancerous cells and producing one or more effector molecules, e.g., immune modulators (s). In certain aspects, the disclosure relates to genetically engineered bacteria that are capable of targeting cancerous cells, particularly in the hypoxic regions of a tumor, and producing one or more effector molecules, e.g., immune modulators (s) under the control of an oxygen level-inducible promoter. In contrast to existing conventional therapies, the hypoxic areas of tumors offer a perfect niche for the growth of anaerobic bacteria, the use of which offers an opportunity for eradication of advanced local tumors in a precise manner, sparing surrounding well-vascularized, normoxic tissue.

Specifically, in some embodiments, the genetically engineered bacteria are capable of producing one or more immune initiators. In some embodiments the genetically engineered bacteria are capable of producing one or more immune sustainers in combination with one or more immune initiators.

In some aspects, the disclosure provides a genetically engineered microorganism that is capable of delivering one or more effector molecules, e.g., immune modulators, such as immune initiators and/or immune sustainers to tumor cells or the tumor microenvironment. In some aspects, the disclosure relates to a genetically engineered microorganism that is delivered systemically, e.g., via any of the delivery means described in the present disclosure, and are capable of producing one or more effector molecules, e.g., immune initiators and/or immune sustainers, as described herein. In some aspects, the disclosure relates to a genetically engineered microorganism that is delivered locally, e.g., via local intra-tumoral administration, and are capable of producing one or more effector molecules, e.g., immune initiators and/or immune sustainers. In some aspects, the compositions and methods disclosed herein may be used to deliver one or more effector molecules, e.g., immune initiators and/or immune sustainers selectively to tumor cells, thereby reducing systemic cytotoxicity or systemic immune dysfunction, e.g., the onset of an autoimmune event or other immune-related adverse event.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

The generation of immunity to cancer is a potentially self-propagating cyclic process which has been referred to as the “Cancer-Immunity Cycle” (Chen and Mellman, Oncology Meets Immunology: The Cancer-Immunity Cycle; Immunity (2013) 39:1-10), and which can lead to the broadening and amplification of the T cell response. The cycle is counteracted by inhibitory factors that lead to immune regulatory feedback mechanisms at various steps of the cycle and which can halt the development or limit the immunity.

The cycle essentially comprises a series of steps which need to occur for an anticancer immune response to be successfully mounted. The cycle includes steps, which must occur for the immune response to be initiated and a second series of events which must occur subsequently, in order for the immune response to be sustained (i.e., allowed to progress and expand and not dampened). These steps have been referred to as the “Cancer-Immunity Cycle” (Chen and Mellman, 2013), and are essentially as follows:

1. Release (Oncolysis) and/or Acquisition of Tumor Cell Contents;

Tumor cells break open and spill their contents, resulting in the release of neoantigens, which are taken up by antigen presentating cells (dendritic cells and macrophages for processing. Alternatively, antigen presenting cells may actively phagocytose tumors cells directly.

2. Activation of Antigen Presenting Cells (APC) (Dendritic Cells and Macrophages);

In addition to the first step described above, the next step must involve release of proinflammatory cytokines or generation of proinflammatory cytokines as a result of release of DAMPs or PAMPs from the dying tumor cells to result in antigen presenting cell activation and subsequently an anticancer T cell response. Antigen presenting cell activation is critical to avoid peripheral tolerance to tumor derived antigens. If properly activated, antigen presenting cells present the previously internalized antigens on their surface in the context of MHCI and MHCII molecules alongside the proper co-stimulatory signals (CD80/86, cytokines, etc.) to prime and activate T cells.

3. Priming and Activation of T cells:

Antigen presentation by DCs and macrophages causes the priming and activation of effector T cell responses against the cancer-specific antigens, which are seen as “foreign” by the immune system. This step is critical to the strength and breadth of the anti-cancer immune response, by determining quantity and quality of T effector cells and contribution of T regulatory cells. Additionally, proper priming of T cells can result in superior memory T cell formation and long lived immunity.

4. Trafficking and Infiltration:

Next, the activated effector T cells must traffic to the tumor and infiltrate the tumor.

5. Recognition of cancer cells by T cells and T cell support, and augmentation and expansion of effector T cell responses:

Once arrived at the tumor site, the T cells can recognize and bind to cancer cells via their T cell receptors (TCR), which specifically bind to their cognate antigen presented within the context of MHC molecules on the cancer cells, and subsequently kill the target cancer cell Killing of the cancer cell releases tumor associated antigens through lysis of tumor cells, and the cycle re-initiates, thereby increasing the volume of the response in subsequent rounds of the cycle. Antigen recognition by either MHC-I or MHC-II restricted T cells can result in additional effector functions, such as the release of chemokines and effector cytokines, further potentiating a robust antitumor response.

6. Overcoming immune suppression:

Finally, overcoming certain deficiencies in the immune response to the cancer and/or overcoming the defense strategy of the cancer, i.e., overcoming the breaks that the cancer employs in fighting the immune response, can be viewed as another critical step in the cycle. In some cases, even though T cell priming and activation has occurred, other immunosuppressive cell subsets are actively recruited and activated to the tumor microenvironement, i.e., regulatory T cells or myloid derived suppressor cells. In other cases, T cells may not receive the right signals to properly home to tumors or may be actively excluded from infiltrating the tumor. Finally, certain mechanisms in the tumor microenvironment exist, which are capable of suppressing or repressing the effector cells that are produced as a result of the cycle. Such resistance mechanisms co-opt immune-inhibitory pathways, often referred to as immune checkpoints, which normally mediate immune tolerance and mitigate cancer tissue damage (see e.g., Pardoll (2012), The blockade of immune checkpoints in cancer immunotherapy; Nature Reviews Cancer volume 12, pages 252-264).

One important immune-checkpoint receptor is cytotoxic T-lymphocyte-associated antigen 4 (CTLA4), which downmodulates the amplitude of T cell activation. Some immune-checkpoint receptors, such as programmed cell death protein 1 (PD1), limit T cell effector functions within tissues. By upregulating ligands for PD1, tumor cells and antigen presenting cells block antitumor immune responses in the tumor microenvironment. Multiple additional immune-checkpoint receptors and ligands, some of which are selectively upregulated in various types of tumor cells, are prime targets for blockade, particularly in combination with approaches that enhance the initiation or activation of antitumor immune responses.

Therapies have been developed to promote and support progression through the cancer-immunity cycle at one or more of the 6 steps. These therapies can be broadly classified as therapies that promote initiation of the immune response and therapies that help sustain the immune response.

As used herein the term “immune initiation” or “initiating the immune response” refers to advancement through the steps which lead to the generation and establishment of an immune response. For example, these steps could include the first three steps of the cancer immunity cycle described above, i.e., the process of antigen aquisition (step (1)), activation of dendritic cells and macrophages (step (2)), and/or the priming and activation of T cells (step (3)).

As used herein the term “immune sustenance” or “sustaining the immune response” refers to the advancement through steps which ensure the immune response is broadened and strengthened over time and which prevent dampening or suppression of the immune response. For example, these steps could include steps 4 through 6 of the cycle described, i.e., T cell trafficking and tumor infiltration, recognition of cancer cells though TCRs, and overcoming immune suppression, i.e., depletion or inhibition of T regulatory cells and preventing the establishment of other active suppression of the effector response.

Accordingly, in some embodiments, the genetically engineered bacteria are capable of modulating, e.g., advancing the cancer immunity cycle by modulating, e.g., activating, promoting supporting, one or more of the steps in the cycle. In some embodiments, the genetically engineered bacteria are capable of modulating, e.g., promoting, steps that modulate, e.g., intensify, the initiation of the immune response. In some embodiments, the genetically engineered bacteria are capable of modulating, e.g., boosting, certain steps within the cycle that enhance sustenance of the immune response. In some embodiments, the genetically engineered bacteria are capable of modulating, e.g., intensifying, the initiation of the immune response and modulating, e.g., enhancing, sustenance of the immune response.

Accordingly, in some embodiments, the genetically engineered bacteria are capable of producing one or more effector molecules, e.g., immune modulators, which modulate, e.g., intensify the initiation of the immune response. Accordingly, in some embodiments, the genetically engineered bacteria are capable of producing one or more effector molecules, e.g., immune modulators, which modulate, e.g., enhance, sustenance of the immune response. Accordingly, in some embodiments, the genetically engineered bacteria are capable of producing one or more effector molecules, e.g., immune modulators, which modulate, e.g., intensify, the initiation of the immune response and one or more one or more effector molecules, e.g., immune modulators, which modulate, e.g., enhance, sustenance of the immune response.

Accordingly, in some embodiments, the genetically engineered bacteria comprise gene sequences encoding one or more effector molecules, e.g., immune modulators, which modulate, e.g., intensify the initiation of the immune response. Accordingly, in some embodiments, the genetically engineered bacteria comprise gene sequences encoding one or more effector molecules, e g., immune modulators, which modulate, e.g., enhance, sustenance of the immune response. Accordingly, in some embodiments, the genetically engineered bacteria comprise gene sequences encoding one or more effector molecules, e.g., immune modulators, which modulate, e.g., intensify, the initiation of the immune response and one or more one or more effector molecules, e.g., immune modulators, which modulate, e.g., enhance, sustenance of the immune response.

An“effector”, “effector substance” or “effector molecule” refers to one or more molecules, therapeutic substances, or drugs of interest. In one embodiment, the “effector” is produced by a modified microorganism, e.g., bacteria. In another embodiment, a modified microorganism capable of producing a first effector described herein is administered in combination with a second effector, e.g., a second effector not produced by a modified microorganism but administered before, at the same time as, or after, the administration of the modified microorganism producing the first effector.

A non-limiting example of such effector or effector molecules are “immune modulators,” which include immune sustainers and/or immune initiators as described herein. In some embodiments, the modified microorganism is capable of producing two or more effector molecules or immune modulators. In some embodiments, the modified microorganism is capable of producing three, four, five, six, seven, eight, nine, or ten effector molecules or immune modulators. In some embodiments, the effector molecule or immune modulator is a therapeutic molecule that is useful for modulating or treating a cancer. In another embodiment, a modified microorganism capable of producing a first immune modulator described herein is administered in combination with a second immune modulator, e.g., a second immune modulator not produced by a modified microorganism but administered before, at the same time as, or after, the administration of the modified microorganism producing the first immune modulator.

In some embodiments, the effector or immune modulator is a therapeutic molecule encoded by at least one gene. In other embodiments, the effector or immune modulator is a therapeutic molecule produced by an enzyme encoded by at least one gene. In alternate embodiments, the effector molecule or immune modulator is a therapeutic molecule produced by a biochemical or biosynthetic pathway encoded by at least one gene. In another rembodiment, the effector molecule or immune modulator is at least one enzyme of a biochemical, biosynthetic, or catabolic pathway encoded by at least one gene. In some embodiments, the effector molecule or immune modulator may be a nucleic acid molecule that mediates RNA interference, microRNA response or inhibition, TLR response, antisense gene regulation, target protein binding (aptamer or decoy oligos), or gene editing, such as CRISPR interference. Other types of effectors and immune modulators are described and listed herein.

Non-limiting examples of effector molecules and/or immune modulators include immune checkpoint inhibitors (e.g., CTLA-4 antibodies, PD-1 antibodies, PDL-1 antibodies), cytotoxic agents (e.g., Cly A, FASL, TRAIL, TNFα, immunostimulatory cytokines and co-stimulatory molecules (e.g., OX40 antibody or OX40L, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectin1, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK), immune stimulatory metabolites and biosynthetic pathway enzymes that produce them (STING agonists, e.g., c-di-AMP, 3′3′-cGAMP, and 2′3′-cGAMP; arginine, tryptophan).

Effectors may also include enzymes or other polypeptides (such as transporters or regulatory proteins) or other modifications (such as inactivation of certain endogenous genes, e.g., auxotrophies), which result in catabolism of immune suppressive or tumor growth promoting metabolites, such as kynurenine, adenosine and ammonia. Non-limiting examples of kynurenine, adenosine, and ammonia consuming circuits are described herein.

Immune modulators include, inter alia, immune initiators and immune sustainers.

As used herein, the term “immune initiator” or “initiator” refers to a class of effectors or molecules, e.g., immune modulators, or substances. Immune initiators may modulate, e.g., intensify or enhance, one or more steps of the cancer immunity cycle, including (1) lysis of tumor cells (oncolysis); (2) activation of APCs (dendritic cells and macrophages); and/or (3) priming and activation of T cells. In one embodiment, an immune initiator may be produced by a modified microorganism, e.g., bacterium, described herein, or may be administered in combination with a modified microorganism of the disclosure. For example, a modified microorganism capable of producing a first immune initiator or immune sustainer described herein is administered in combination with a second immune initiator, e.g., a second immune initiator not produced by a modified microorganism but administered before, at the same time as, or after, the administration of the modified microorganism producing the first immune initiator or immune sustainer. Non-limiting examples of such immune initiators are described in further detail herein.

In some embodiments, an immune initiator is a therapeutic molecule encoded by at least one gene. Non-limiting examples of such therapeutic molecules are described herein and include, but are not limited to, cytokines, chemokines, single chain antibodies (agonistic or antagonistic), ligands (agonistic or antagonistic), co-stimulatory receptors/ligands and the like. In another embodiment, an immune initiator is a therapeutic molecule produced by an enzyme encoded by at least one gene. Non-limiting examples of such enzymes are described herein and include, but are not limited to, DacA and cGAS, which produce a STING agonist. In another embodiment, an immune initiator is at least one enzyme of a biosynthetic pathway encoded by at least one gene. Non-limiting examples of such biosynthetic pathways are described herein and include, but are not limited to, enzymes involved in the production of arginine. In another embodiment, an immune initiator is at least one enzyme of a catabolic pathway encoded by at least one gene. Non-limiting examples of such catabolic pathways are described herein and include, but are not limited to, enzymes involved in the catabolism of a harmful metabolite. In another embodiment, an immune initiator is at least one molecule produced by at least one enzyme of a biosynthetic pathway encoded by at least one gene. In another embodiment, an immune initiator is a therapeutic molecule produced by metabolic conversion, i.e., the immune initiator is a metabolic converter. In other embodiments, the immune initiator may be a nucleic acid molecule that mediates RNA interference, microRNA response or inhibition, TLR response, antisense gene regulation, target protein binding (aptamer or decoy oligos), gene editing, such as CRISPR interference.

The term “immune initiator” may also refer to any modifications, such as mutations or deletions, in endogenous genes. In some embodiments, the bacterium is engineered to express the biochemical, biosynthetic, or catabolic pathway. In some embodiments, the bacterium is engineered to produce a second messenger molecule.

In a broader sense, a microorganism, e.g., bacterium, may be referred to herein as an “immune initiator microorganism” when it is capable of producing an “immune initiator.”

In specific embodiments, the modified microorganism is capable of producing one or more immune initiators, which modulate, e.g., intensify, one or more of steps (1) lysis of tumor cells and/or uptake of tumor antigens, (2), activation of APCs and/or (3) priming and activation of T cells. In some embodiments, the modified microorganism comprises gene circuitry for the production of one or more immune initiators, which modulate, e.g., intensify, one or more of steps (1) lysis of tumor cells and/or uptake of tumor antigens, (2) activation of APCs and/or (3) priming and activation of T cells. In some embodiments, the genetically engineered bacteria comprise one or more genes encoding one or more immune initiators, which modulate, e.g., intensify, one or more of steps (1) oncolysis and/or uptake of tumor antigens, (2) activation of APCs and/or (3) priming and activation of T cells. Any immune initiator may be combined with one or more additional same or different immune initiator(s), which modulate the same or a different step in the cancer immunity cycle.

In one embodiment, the modified microorganisms produce one or more immune initiators which modulate oncolysis or tumor antigen uptake (step (1)). Non-limiting examples of immune initiators which modulate antigen acquisition are described herein and known in the art and include but are not limited to lytic peptides, CD47 blocking antibodies, SIRP-alpha and variants, TNFα, IFN-γ and 5FU. In one embodiment, the modified microorganisms produce one or more immune initiators which modulate activation of APCs (step (2)). Non-limiting examples of immune initiators which modulate activation of APCs are described herein and known in the art and include but are not limited to Toll-like receptor agonists, STING agonists, CD40L, and GM-CSF. In one embodiment, the modified microorganisms produce one or more immune initiators which modulate, e.g., enhance, priming and activation of T cells (step (3)). Non-limiting examples of immune initiators which modulate, e.g., enhance, priming and activation of T cells are described herein and known in the art and include but are not limited to an anti-OX40 antibody, OXO40L, an anti-41BB antibody, 41BBL, an anti-GITR antibody, GITRL, anti-CD28 antibody, anti-CTLA4 antibody, anti-PD1 antibody, anti-PDL1 antibody, IL-15, and IL-12, etc.

As used herein the term “immune sustainer” or “sustainer” refers to a class of effectors or molecules, e.g., immune modulators, or substances. Immune sustainers may modulate, e.g., boost or enhance, one or more steps of the cancer immunity cycle, including (4) trafficking and infiltration; (5) recognition of cancer cells by T cells and T cell support; and/or (6) the ability to overcome immune suppression. In one embodiment, the immune sustainer may be produced by the modified microorganisms, e.g., bacteria, described herein. In another embodiment, an immune sustainer may be administered in combination with a modified microorganism described herein. For example, a modified microorganism capable of producing a first immune initiator or immune sustainer described herein is administered in combination with a second immune sustainer, e.g., a second immune sustainer not produced by a modified microorganism but administered before, at the same time as, or after, the administration of the modified microorganism producing the first immune initiator or immune sustainer.

In some embodiments, the immune sustainer is a therapeutic molecule encoded by at least one gene. Non-limiting examples of such therapeutic molecules are described herein and include cytokines, chemokines, single chain antibodies (agonistic or antagonistic), ligands (agonistic or antagonistic), and the like. In another embodiment, an immune sustainer is a therapeutic molecule produced by an enzyme encoded by at least one gene. Non-limiting examples of such enzymes are described herein and include, but are not limited to, those described in Table 8. In another embodiment, an immune sustainer is at least one enzyme of a biosynthetic pathway or a catabolic pathway encoded by at least one gene. Non-limiting examples of such biosynthetic pathways are described herein and include, but are not limited to, enzymes involved in the production of arginine; and non-limiting examples of such catabolic pathways are described herein and include, but are not limited to, enzymes involved in the catalysis of kynurenine or enzymes involved in the catalysis of adenosine. In another embodiment, an immune sustainer is at least one molecule produced by at least one enzyme of a biosynthetic, biochemical, or catabolic pathway encoded by at least one gene. In another embodiment, an immune sustainer is a therapeutic molecule produced by metabolic conversion, i.e., the immune initiator is a metabolic converter. In other embodiments, the immune sustainer may be a nucleic acid molecule that mediates RNA interference, microRNA response or inhibition, TLR response, antisense gene regulation, target protein binding (aptamer or decoy oligos), gene editing, such as CRISPR interference.

In specific embodiments, the modified microorganisms are capable of breaking down a harmful metabolite, e.g., a metabolite which promotes cell division, proliferation, cancer growth and/or suppresses the immune system, e.g., by preventing progression through the cancer immunity cycle. Accordingly, the term “immune sustainer” may also refer to the reduction or elimination of a harmful molecule. In such instances, the term “immune sustainer” may also be used to refer to the one or more enzymes of the catabolic pathway which breaks down the harmful metabolite, which may be encoded by one or more gene(s). The term “immune sustainer” may refer to the circuitry encoding the catabolic enzymes, circuitry for producing the catabolic enzymes, or the catabolic enzymes expressed by the microorganism.

The term “immune sustainer” may also refer to any modifications, such as mutations or deletions, in endogenous genes. In some embodiments, the microorganism is modified to express the biochemical, biosynthetic, or catabolic pathway. In some embodiments, the microorganism is engineered to produce a second messenger molecule.

In a broader sense, a microorganism, e.g., bacterium, may be referred to as an “immune sustainer microorganism” when it is capable of producing an “immune sustainer.”

In some embodiments, the modified microorganisms are capable of producing one or more immune sustainers, which modulate, e.g., boost, one or more of steps (4) T cell trafficking and infiltration, (5) recognition of cancer cells by T cells and/or T cell support and/or (6) the ability to overcome immune suppression. Any immune sustainer may be combined with one or more additional immune sustainer(s), which modulate the same or a different step. In some embodiments, the modified microorganisms comprise gene circuitry for the production of one or more immune sustainers, which modulate, e.g., boost, one or more of steps (4) T cell trafficking and infiltration, (5) recognition of cancer cells by T cells and/or T cell support and/or (6) the ability to overcome immune suppression. In some embodiments, the modified microorganisms comprise one or more genes encoding one or more immune sustainers, which modulate, e.g., boost, one or more of steps (4) T cell trafficking and infiltration, (5) recognition of cancer cells by T cells and/or T cell support and/or (6) the ability to overcome immune suppression.

In one embodiment, the modified microorganisms produce one or more immune sustainers which modulate T cell trafficking and infiltration (step (4)). Non-limiting examples of immune sustainers which modulate T cell trafficking and infiltration are described herein and known in the art and include, but are not limited to, chemokines such as CXCL9 and CXCL10 or upstream activators which induce the expression of such cytokines. In one embodiment, the modified microorganisms produce one or more immune sustainers which modulate recognition of cancer cells by T cells and T cell support (step (5)). Non-limiting examples of immune sustainers which modulate recognition of cancer cells by T cells and T cell support are described herein and known in the art and include, but are not limited to, anti-PD1/PD-L1 antibodies (antagonistic), anti-CTLA-4 antibodies (antagonistic), kynurenine consumption, adenosine consumption, anti-OX40 antibodies (agonistic), anti-41BB antibodies (agonistic), and anti-GITR antibodies (agonistic). In one embodiment, the modified microorganisms produce one or more immune sustainers which modulate, e.g., enhance, the ability to overcome immune suppression (step (6)). Non-limiting examples of immune sustainers which modulate, e.g., enhance, the ability to overcome immune suppression are described herein and known in the art and include, but are not limited to, IL-15 and IL-12 and variants thereof.

Any one or more immune initiator(s) may be combined any one or more immune sustainer(s). Accordingly, in some embodiments, the modified microorganisms are capable of producing one or more immune initiators which modulate, e.g., intensify, one or more of steps (1) oncolysis, (2) activation of APCs and/or (3) priming and activation of T cells in combination with one or more immune sustainers, which modulate, e.g., boost, one or more of steps (4) T cell trafficking and infiltration, (5) recognition of cancer cells by T cells and/or T cell support and/or (6) the ability to overcome immune suppression.

In some embodiments, certain immune modulators act at multiple stages of the cancer immunity cycle, e.g., one or more stages of immune initiation, or one or more of immune sustenance, or at one or more stages of immune initiation and at one o more stages of immune sustenance.

As used herein a “metabolic conversion” refers to a chemical transformation within the cell, e.g., the bacterial cell, which is the result of an enzyme-catalyzed reaction. The enzyme-catalyze reaction can be either biosynthetic or catabolic in nature.

As used herein, the term “metabolic converter” refers to a biosynthetic or catabolic circuit, i.e., a circuit which comprises gene(s) encoding one or more enzymes, which catalyze a chemical transformation, i.e., which consume, produce or convert a metabolite. In one embodiment, the gene(s) are non-native genes. In another embodiment, the gene(s) may be encoded by native genes, but the circuit is further modified to comprise one or more non-native genes and/or one or more non-native auxotrophies. In some embodiments, the term “metabolic converter” refers to the at least one molecule produced by the at least one enzyme of a biosynthetic pathway encoded by at least one gene.

“Metabolic converter” also refers to the biosynthetic or catabolic enzymes encoded by a circuit as well as any modifications, such as mutations or deletions, in endogenous genes. The term “metabolic converter” may also refer to the one or more gene(s) encoding the catabolic enzymes and/or modifications of endogenous genes. For example, a metabolic converter can consume a toxic or immunosuppressive metabolite or produce an anti-cancer metabolite, or both. Non-limiting examples of metabolic converters include kynurenine consumers, adenosine consumers, arginine producers and/or ammonia consumers, i.e., circuitry, which encodes enzymes for the consumption of kynurenine or adenosine or for the production of arginine and/or consumption of ammonia.

In a broader sense, a microorganism, e.g. bacterium, may be referred to herein as a “metabolic converter microorganism” or “metabolic converter bacterium” when it comprises or is capable of producing a “metabolic converter.”

As used herein, the term “partial regression” refers to an inhibition of growth of a tumor, and/or the regression of a tumor, e.g., in size, after administration of the modified microorganism(s) and/or immune modulator(s) to a subject having the tumor. In one embodiment, a “partial regression” may refer to a regression of a tumor, e.g., in size, by at least about 5%, at least about 10%, at least about 15%, at least about 20%, at least about 25%, at least about 30%, at least about 40%, at least about 50%, at least about 60%, at least about 70%, at least about 80%, or at least about 90%. In another embodiment, a “partial regression” may refer to a decrease in the size of a tumor by at least about 5%, at least about 10%, at least about 15%, at least about 20%, at least about 25%, at least about 30%, at least about 40%, at least about 50%, at least about 60%, at least about 70%, at least about 75%, at least about 80%, or at least about 90%. In one embodiment, “partial regression” refers to the regression of a tumor, e.g., in size, but wherein the tumor is still detectable in the subject.

As used herein, the term “complete regression” refers to a complete regression of a tumor, e.g., in size, after administration of the modified microorganism(s) and/or immune modulator(s) to the subject having the tumor. When “complete regression” occurs the tumor is undetectable in the subject

As used herein, the term “percent response” refers to a percentage of subjects in a population of subjects who exhibit either a partial regression or a complete regression, as defined herein, after administration of a modified microorganism(s) and/or immune modulator(s). For example, in one embodiment, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of subjects in a population of subjects exhibit a partial response or a complete response.

As used herein, the term “stable disease” refers to a cancer or tumor that is neither growing nor shrinking “Stable disease” also refers to a disease state where no new tumors have developed, and a cancer or tumor has not spread to any new region or area of the body, e.g., by metastiasis.

“Intratumoral administration” is meant to include any and all means for microorganism delivery to the intratumoral site and is not limited to intratumoral injection means. Examples of delivery means for the engineered microorganisms is discussed in detail herein.

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute).

As used herein, “abscopal” and “abscopal effect” refers to an effect in which localized treatment of a tumor not only shrinks or otherwise affects the tumor being treated, but also shrinks or otherwise affects other tumors outside the scope of the localized treatment. In some embodiments, the genetically engineered bacteria may elicit an abscopal effect. In some embodiments, no abscopal effect is observed upon administration of the genetically engineered bacteria.

In any of these embodiments in which abscopal effect is observed, timing of tumor growth in a tumor of the same type which is distal to the administration site is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days of the same type relative to the tumor growth (tumor volume) in a naive animal or subject.

In any of these embodiments in which an abscopal effect is observed, timing of tumor growth as measured in tumor volume in a distal tumor of the same type is delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which abscopal effect is observed, timing of tumor growth as measured in tumor volume in a tumor distal to the administration site of the same type is delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject. In this example, “tumor re-challenge” may also include metastasis formation which may occur in a subject at a certain stage of cancer progression.

Immunological memory represents an important aspect of the immune response in mammals. Memory responses form the basis for the effectiveness of vaccines against cancer cells. As used herein, the term “immune memory” or “immunological memory” refers to a state in which long-lived antigen-specific lymphocytes are available and are capable of rapidly mounting responses upon repeat exposure to a particular antigen. The importance of immunological memory in cancer immunotherapy is known, and the trafficking properties and long-lasting anti-tumor capacity of memory T cells play a crucial role in the control of malignant tumors and prevention of metastasis or reoccurrence Immunological memory exists for both B lymphocytes and for T cells, and is now believed to exist in a large variety of other immune cells, including NK cells, macrophages, and monocytes. (see e.g., Farber et al., Immunological memory: lessons from the past and a look to the future (Nat. Rev. Immunol. (2016) 16: 124-128). Memory B cells are plasma cells that are able to produce antibodies for a long time. The memory B cell has already undergone clonal expansion and differentiation and affinity maturation, so it is able to divide multiple times faster and produce antibodies with much higher affinity. Memory T cells can be both CD4+ and CD8+. These memory T cells do not require further antigen stimulation to proliferate therefore they do not need a signal via MHC.

Immunological memory can, for example, be measured in an animal model by re-challenging the animal model upon achievement of complete regression upon treatment with the modified microorganism. The animal is then implanted with cancer cells from the cancer cell line and growth is monitored and compared to an age matched naïve animal of the same type which had not previously been exposed to the tumor. Such a tumor re-challenge is used to demonstrate systemic and long term immunity against tumor cells and may represent the ability to fight off future recurrence or metastasis formation. Such an experiment is described herein using the A20 tumor model in the Examples. Immunological memory would prevent or slow the reoccurrence of the tumor in the re-challenged animal relative to the naïve animal. On a cellular level, formation of immunological memory can be measured by expansion and/or persistence of tumor antigen specific memory or effector memory T cells.

In some embodiments, immunological memory is achieved in a subject upon administration of the modified microorganisms described herein. In some embodiments, immunological memory is achieved cancer patient upon administration of the modified microorganisms described herein.

In some embodiments, a complete response is achieved in a subject upon administration of the modified microorganisms described herein. In some embodiments, a complete response is achieved in a cancer patient upon administration of the modified microorganisms described herein.

In some embodiments, a complete remission is achieved in a subject upon administration of the modified microorganisms described herein. In some embodiments, a complete remission is achieved in a cancer patient upon administration of the modified microorganisms described herein.

In some embodiments, a partial response is achieved in a subject upon administration of the modified microorganisms described herein. In some embodiments, a partial response is achieved in a cancer patient upon administration of the modified microorganisms described herein.

In some embodiments, stable disease is achieved in a subject upon administration of the modified microorganisms described herein. In some embodiments, a partial response is achieved in a cancer patient upon administration of the modified microorganisms described herein.

In some embodiments, a subset of subjects within a group achieves a partial or complete response upon administration of the modified microorganisms described herein. In some embodiments, a subset of patients within a group achieve a partial or complete response upon administration of the modified microorganisms described herein.

In any of these embodiments in which immunological memory is observed, timing of tumor growth is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days in a tumor re-challenge relative to the tumor growth (tumor volume) in a naive animal or subject.

In any of these embodiments in which immunological memory is observed, timing of tumor growth as measured in tumor volume delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which immunological memory is observed, timing of tumor growth as measured in tumor volume delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject.

As used herein, “hot tumors” refer to tumors, which are T cell inflamed, i.e., associated with a high abundance of T cells infiltrating into the tumor. “Cold tumors” are characterized by the absence of effector T cells infiltrating the tumor and are further grouped into “immune excluded” tumors, in which immune cells are attracted to the tumor but cannot infiltrate the tumor microenvironment, and “immune ignored” phenotypes, in which no recruitement of immune cells occurs at all (further reviewed in Van der Woude et al., Migrating into the Tumor: a Roadmap for T Cells. Trends Cancer. 2017 November; 3(11):797-808).

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion (Groot et al., 2007).

As used herein, “payload” refers to one or more molecules of interest to be produced by a genetically engineered microorganism, such as a bacteria or a virus. In some embodiments, the payload is a therapeutic payload, e.g., an effector, or immune modulator, e.g., immune initiator or immune sustainer. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments, the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere.

In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1 5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties.

In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nonaerobic, microoxic, hypoxic, anoxic, and/or anaerobic conditions. For example, Table 1 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>.

In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated.

In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.).

The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 1

| Compartment | Oxygen Tension |
|---|---|
| stomach | ~60 torr (e.g., 58 +/− 15 torr) |
| duodenum and first part of | ~30 torr (e.g., 32 +/− 8 torr); |
| jejunum | ~20% oxygen in ambient air |
| Ileum (mid-small intestine) | ~10 torr; ~6% oxygen in ambient |
| | air (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

As used herein, the term “gene” or “gene sequence” refers to any sequence expressing a polypeptide or protein, including genomic sequences, cDNA sequences, naturally occurring sequences, artificial sequences, and codon optimized sequences. The term “gene” or “gene sequence” inter alia includes modification of endogenous genes, such as deletions, mutations, and expression of native and non-native genes under the control of a promoter that that they are not normally associated with in nature.

As used herein the terms “gene cassette” and “circuit” or “circuitry” inter alia refers to any sequence expressing a polypeptide or protein, including genomic sequences, cDNA sequences, naturally occurring sequences, artificial sequences, and codon optimized sequences includes modification of endogenous genes, such as deletions, mutations, and expression of native and non-native genes under the control of a promoter that that they are not normally associated with in nature.

An antibody generally refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer composed of two identical pairs of polypeptide chains, each pair having one “light” (about 25 kD) and one “heavy” chain (about 50-70 kD), connected through a disulfide bond.

As used herein, the term “antibody” or “antibodies “is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, V_{H}H fragments), heavy chain antibodies (HCAb), nanobodies, diabodies, and minibodies. Antibodies can have more than one binding specificity, e.g. be bispecific. The term “antibody” is also meant to include so-called antibody mimetics, i.e., which can specifically bind antigens but do not have an antibody-related structure.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. In some embodiments, the polypeptide is produced by the genetically engineered bacteria of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids.

An “isolated” polypeptide or a fragment, variant, or derivative thereof refers to a polypeptide that is not in its natural milieu No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: −Ala, Pro, Gly, Gln, Asn, Ser, Thr; −Cys, Ser, Tyr, Thr; −Val, Ile, Leu, Met, Ala, Phe; −Lys, Arg, His; −Phe, Tyr, Trp, His; and −Asp, Glu.

In any of these combination embodiments, the genetically engineered bacteria may comprise gene sequence(s) encoding one or more fusion proteins. In some embodiments, the genetically engineered bacteria comprise gene sequence(s) encoding an effector, e.g., an immune modulator, fused to a stabilizing polypeptide. Such stabilizing polypeptides are known in the art and include Fc proteins. In some embodiments, the fusion proteins encoded by the genetically engineered bacteria are Fc fusion proteins, such as IgG Fc fusion proteins or IgA Fc fusion proteins.

In some embodiments, an immune modulator, is covalently fused to the stabilizing polypeptide through a peptide linker or a peptide bond. In some embodiments, the stabilizing polypeptide comprises an immunoglobulin Fc polypeptide. In some embodiments, the immunoglobulin Fe polypeptide comprises at least a portion of an immunoglobulin heavy chain CH2 constant region. In some embodiments, the immunoglobulin Fc polypeptide comprises at least a portion of an immunoglobulin heavy chain CH3 constant region. In some embodiments, the immunoglobulin Fc polypeptide comprises at least a portion of an immunoglobulin heavy chain CH1 constant region. In some embodiments, the immunoglobulin Fc polypeptide comprises at least a portion of an immunoglobulin variable hinge region. In some embodiments, the immunoglobulin Fe polypeptide comprises at least a portion of an immunoglobulin variable hinge region, immunoglobulin heavy chain CH2 constant region and an immunoglobulin heavy chain CH3 constant region. The genetically engineered bacterium of any of claims 2-64, and any of claims 112-122, wherein the immunoglobulin Fe polypeptide is a human IgG Fe polypeptide. In some embodiments, the immunoglobulin Fc polypeptide is a human IgG4 Fe polypeptide. In some embodiments, the linker comprises a glycine rich peptide. In some embodiments, the glycine rich peptide comprises the sequence [GlyGlyGlyGlySer]n where n is 1, 2, 3, 4, 5 or 6. In some embodiments, the fusion protein comprises a SIRPα IgG FC fusion polypeptide. In some embodiments, the fusion protein comprises a SIRPα IgG4 Fc polypeptide. In some embodiments, the glycine rich peptide linker comprises the sequence SGGGGSGGGGSGGGGS. In some embodiments, the N terminus of SIRPα is covalently fused to the C terminus of a IgG4 Fc through the peptide linker comprising SGGGGSGGGGSGGGGS.

In some embodiments, the genetically engineered bacteria comprise one or more gene sequences encoding components of a multimeric polypeptide. In some embodiments, the polypeptide is a dimer. Non-limiting example of a dimeric proteins include cytokines, such as IL-15 (heterodimer). In some embodiments, genetically engineered bacteria comprise one or more gene(s) encoding one or more polypeptides wherein the one or more polypeptides comprise a first monomer and a second monomer. In some embodiments, the first monomer polypeptide is covalently linked to a second monomer polypeptide through a peptide linker or a peptide bond. In some embodiments, the linker comprises a glycine rich peptide. In some embodiments, the first and the second monomer have the same polypeptide sequence. In some embodiments, the first and the second monomer have each have a different polypeptide sequence. In some embodiments, the first monomer is a IL-12 p35 polypeptide and the second monomer is a IL-12 p40 polypeptide. In some embodiments, the linker comprises GGGGSGGGS.

In some embodiments, the genetically engineered bacteria encode a hIGg4 fusion protein which comprises a hIgG4 portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1117. In another embodiment, the hIgG4 portion comprises SEQ ID NO: 1117. In yet another embodiment, the hIgG4 portion of the polypeptide expressed by the genetically engineered bacteria consists of SEQ ID NO: 1117.

In some embodiments, the nucleic acid encoding a fusion protein, such as an hIGg4 fusion protein, comprises a sequence which has at least about 80%, at least about 85%, at least about 90%, at least about 95%, or at least about 99% identity to a SEQ ID NO: 1103. In some embodiments, the nucleic acid encoding a fusion protein, comprises SEQ ID NO: 1103. In some embodiments, nucleic acid portion encoding hIgG4 consists of a SEQ ID NO: 1103.

In some embodiments, the genetically engineered bacteria encode a fusion protein which comprises a linker portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1121. In another embodiment, the linker portion comprises SEQ ID NO: 1121. In yet another embodiment, the linker portion of the polypeptide expressed by the genetically engineered bacteria consists of SEQ ID NO: 1121.

In some embodiments, effector function of an immune modulator can be improved through fusion to another polypeptide that facilitates effector function. A non-limiting example of such a fusion is the fusion of IL-15 to the Sushi domain of IL-15Ralpha, as described herein. In some embodiments, accordingly, a first monomer polypeptide is a IL-15 monomer and the second monomer is a IL-15R alpha sushi domain polypeptide.

In any of these embodiments and all combination embodiments, the genetically engineered bacteria comprise gene sequence(s) encoding one or more secretion tags described herein. In any of these embodiments, the genetically engineered bacteria comprise one or more mutations in an endogenous membrane associated protein allowing for the diffusible outer membrane phenotype. Suitable outer membrane mutations are described herein.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety. In some embodiments, the linker is a glycine rich linker. In some embodiments, the linker is (Gly-Gly-Gly-Gly-Ser)n. In some embodiments, the linker comprises SEQ ID NO: 979.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter cilia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the immune modulator from the microbial, e.g., bacterial cytoplasm. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-limiting examples of secretion systems are described herein.

As used herein, the term “transporter” is meant to refer to a mechanism, e.g., protein or proteins, for importing a molecule into the microorganism from the extracellular milieu.

The immune system is typically most broadly divided into two categories-innate immunity and adaptive immunity-although the immune responses associated with these immunities are not mutually exclusive. “Innate immunity” refers to non-specific defense mechanisms that are activated immediately or within hours of a foreign agent's or antigen's appearance in the body. These mechanisms include physical barriers such as skin, chemicals in the blood, and immune system cells, such as dendritic cells (DCs), leukocytes, phagocytes, macrophages, neutrophils, and natural killer cells (NKs), that attack foreign agents or cells in the body and alter the rest of the immune system to the presence of the foreign agents. During an innate immune response, cytokines and chemokines are produced which in combination with the presentation of immunological antigens, work to activate adaptive immune cells and initiate a full blown immunologic response. “Adaptive immunity” or “acquired immunity” refers to antigen-specific immune response. The antigen must first be processed or presented by antigen presenting cells (APCs). An antigen-presenting cell or accessory cell is a cell that displays antigens directly or complexed with major histocompatibility complexes (MHCs) on their surfaces. Professional antigen-presenting cells, including macrophages, B cells, and dendritic cells, specialize in presenting foreign antigen to T helper cells in a MHC-II restricted manner, while other cell types can present antigen originating inside the cell to cytotoxic T cells in a MHC-I restricted manner. Once an antigen has been presented and recognized, the adaptive immune system activates an army of immune cells specifically designed to attack that antigen. Like the innate system, the adaptive system includes both humoral immunity components (B lymphocyte cells) and cell-mediated immunity (T lymphocyte cells) components. B cells are activated to secrete antibodies, which travel through the bloodstream and bind to the foreign antigen. Helper T cells (regulatory T cells, CD4+ cells) and cytotoxic T cells (CTL, CD8+ cells) are activated when their T cell receptor interacts with an antigen-bound MHC molecule. Cytokines and co-stimulatory molecules help the T cells mature, which mature cells, in turn, produce cytokines which allows the production of priming and expansion of additional T cells sustaining the response. Once activated, the helper T cells release cytokines which regulate and direct the activity of different immune cell types, including APCs, macrophages, neutrophils, and other lymphocytes, to kill and remove targeted cells. Helper T cells also secrete extra signals that assist in the activation of cytotoxic T cells which also help to sustain the immune response. Upon activation, CTL undergoes clonal selection, in which it gains functions, divides rapidly to produce an army of activated effector cells, and forms long-lived memory T cells ready to rapidly respond to future threats. Activated CTL then travels throughout the body searching for cells that bear that unique MHC Class I and antigen. The effector CTLs release cytotoxins that form pores in the target cell's plasma membrane, causing apoptosis. Adaptive immunity also includes a “memory” that makes future responses against a specific antigen more efficient. Upon resolution of the infection, T helper cells and cytotoxic T cells die and are cleared away by phagocytes, however, a few of these cells remain as memory cells. If the same antigen is encountered at a later time, these memory cells quickly differentiate into effector cells, shortening the time required to mount an effective response.

An “immune checkpoint inhibitor” or “immune checkpoint” refers to a molecule that completely or partially reduces, inhibits, interferes with, or modulates one or more immune checkpoint proteins. Immune checkpoint proteins regulate T-cell activation or function, and are known in the art. Non-limiting examples include CTLA-4 and its ligands CD 80 and CD86, and PD-1 and its ligands PD-L1 and PD-L2 Immune checkpoint proteins are responsible for co-stimulatory or inhibitory interactions of T-cell responses, and regulate and maintain self-tolerance and physiological immune responses.

A “co-stimulatory” molecule or “co-stimulator” is an immune modulator that increase or activates a signal that stimulates an immune response or inflammatory response.

As used herein, a genetically engineered microorganism, e.g., engineered bacterium, or immune modulator that “inhibits” cancerous cells refers to a bacterium or virus or molecule that is capable of reducing cell proliferation, reducing tumor growth, and/or reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to control, e.g., an untreated control or an unmodified microorganism of the same subtype under the same conditions.

As used herein, a genetically engineered microorganism, e.g., engineered bacterium, or immune modulator that “inhibits” a biological molecule, such as an immune modulator, e.g., cytokine, chemokine, immune modulatory metabolite, or any other immune modulatory agent, factor, or molecule, refers to a bacterium or virus or immune modulator that is capable of reducing, decreasing, or eliminating the biological activity, biological function, and/or number of that biological molecule, as compared to control, e.g., an untreated control or an unmodified microorganism of the same subtype under the same conditions.

As used herein, a genetically engineered microorganism, e.g., engineered bacterium, or immune modulator that “activates” or “stimulates” a biological molecule, e.g., cytokine, chemokine, immune modulatory metabolite, or any other immune modulatory agent, factor, or molecule, refers to a bacterium or virus or immune modulator that is capable of activating, increasing, enhancing, or promoting the biological activity, biological function, and/or number of that biological molecule, as compared to control, e.g., an untreated control or an unmodified microorganism of the same subtype under the same conditions.

“Bacteria for intratumoral administration” refer to bacteria that are capable of directing themselves to cancerous cells. Bacteria for intratumoral administration may be naturally capable of directing themselves to cancerous cells, necrotic tissues, and/or hypoxic tissues. In some embodiments, bacteria that are not naturally capable of directing themselves to cancerous cells, necrotic tissues, and/or hypoxic tissues are genetically engineered to direct themselves to cancerous cells, necrotic tissues, and/or hypoxic tissues. Bacteria for intratumoral administration may be further engineered to enhance or improve desired biological properties, mitigate systemic toxicity, and/or ensure clinical safety. These species, strains, and/or subtypes may be attenuated, e.g., deleted for a toxin gene. In some embodiments, bacteria for intratumoral administration have low infection capabilities. In some embodiments, bacteria for intratumoral administration are motile. In some embodiments, the bacteria for intratumoral administration are capable of penetrating deeply into the tumor, where standard treatments do not reach. In some embodiments, bacteria for intratumoral administration are capable of colonizing at least 20%, at least 30%, at least 40%, at least 50%, at least 60%, at least 70%, at least 80%, at least 90%, or at least 95% of a malignant tumor. Examples of bacteria for intratumoral administration include, but are not limited to, Bifidobacterium, Caulobacter, Clostridium, Escherichia coli, Listeria, Mycobacterium, Salmonella, Streptococcus, and Vibrio, e.g., Bifidobacterium adolescentis, Bifidobacterium bifidum, Bifidobacterium breve UCC2003, Bifidobacterium infantis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera (Cronin et al., 2012; Forbes, 2006; Jain and Forbes, 2001; Liu et al., 2014; Morrissey et al., 2010; Nuno et al., 2013; Patyar et al., 2010; Cronin, et al., Mol Ther 2010; 18:1397-407). In some embodiments, the bacteria for intratumoral administration are non-pathogenic bacteria. In some embodiments, intratumoral administration is done via injection.

“Microorganism” refers to an organism or microbe of microscopic, submicroscopic, or ultramicroscopic size that typically consists of a single cell. Examples of microorganisms include bacteria, viruses, parasites, fungi, certain algae, protozoa, and yeast. In some aspects, the microorganism is modified (“modified microorganism”) from its native state to produce one or more effectors or immune modulators. In certain embodiments, the modified microorganism is a modified bacterium. In some embodiments, the modified microorganism is a genetically engineered bacterium. In certain embodiments, the modified microorganism is a modified yeast. In other embodiments, the modified microorganism is a genetically engineered yeast.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to certain strains belonging to the genus Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered or programmed to enhance or improve probiotic properties.

As used herein, an “oncolytic virus “(OV) is a virus having the ability to specifically infect and lyse cancer cells, while leaving normal cells unharmed. Oncolytic viruses of interest include, but are not limited to adenovirus, Coxsackie, Reovirus, herpes simplex virus (HSV), vaccinia, fowl pox, vesicular stomatitis virus (VSV), measles, and Parvovirus, and also includes rabies, west nile virus, New castle disease and genetically modified versions thereof. A non-limiting example of an OV is Talimogene Laherparepvec (T-VEC), the first oncolytic virus to be licensed by the FDA as a cancer therapeutic.

“Operably linked” refers a nucleic acid sequence, e.g., a gene encoding an enzyme for the production of a STING agonist, e.g., a diadenylate cyclase or a c-di-GAMP synthase, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region.

“Exogenous environmental condition(s)” refer to setting(s) or circumstance(s) under which the promoter described herein is induced. In some embodiments, the exogenous environmental conditions are specific to a malignant growth containing cancerous cells, e.g., a tumor. The phrase “exogenous environmental conditions” is meant to refer to the environmental conditions external to the intact (unlysed) engineered microorganism, but endogenous or native to tumor environment or the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are low-oxygen, microaerobic, or anaerobic conditions, such as hypoxic and/or necrotic tissues. Some solid tumors are associated with low intracellular and/or extracellular pH; in some embodiments, the exogenous environmental condition is a low-pH environment. In some embodiments, the genetically engineered microorganism of the disclosure comprise a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 2.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 2
Examples of transcription factors and
responsive genes and regulatory regions

| Transcription | Examples of responsive genes, |
|---|---|
| Factor | promoters, and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered bacteria of the disclosure comprise a gene that is operably linked to a directly or indirectly inducible promoter that is not associated with said gene in nature, e.g., an FNR-responsive promoter (or other promoter described herein) operably linked to a gene encoding an immune modulator.

In one embodiment, the effector, or immune modulator, is a therapeutic molecule encoded by at least one non-native gene. In one embodiment, the effector, or immune modulator, is a therapeutic molecule produced by an enzyme encoded by at least one non-native gene. In one embodiment, the effector, or immune modulator, is at least one enzyme of a biosynthetic pathway encoded by at least one non-native gene. In another embodiment, the effector, or immune modulator, is at least one molecule produced by at least one enzyme of a biosynthetic pathway encoded by at least one non-native gene.

In one embodiment, the immune initiator is a therapeutic molecule encoded by at least one non-native gene. In one embodiment, the immune initiator is a therapeutic molecule produced by an enzyme encoded by at least one non-native gene. In one embodiment, the immune initiator is at least one enzyme of a biosynthetic pathway encoded by at least one non-native gene. In another embodiment, the immune initiator is at least one molecule produced by at least one enzyme of a biosynthetic pathway encoded by at least one non-native gene.

In one embodiment, the immune sustainer is a therapeutic molecule encoded by at least one non-native gene. In one embodiment, the immune sustainer is a therapeutic molecule produced by an enzyme encoded by at least one non-native gene. In one embodiment, the immune sustainer is at least one enzyme of a biosynthetic pathway encoded by at least one non-native gene. In another embodiment, the immune sustainer is at least one molecule produced by at least one enzyme of a biosynthetic pathway encoded by at least one non-native gene.

“Constitutive promoter” refers to a promoter that is capable of facilitating continuous transcription of a coding sequence or gene under its control and/or to which it is operably linked. Constitutive promoters and variants are well known in the art and non-limiting examples of constitutive promoters are described herein and in International Patent Application PCT/US2017/013072, filed Jan. 11, 2017 and published as WO2017/123675, the contents of which is herein incorporated by reference in its entirety. In some embodiments, such promoters are active in vitro, e.g., under culture, expansion and/or manufacture conditions. In some embodiments, such promoters are active in vivo, e.g., in conditions found in the in vivo environment, e.g., the gut and/or the tumor microenvironment.

As used herein, “stably maintained” or “stable” bacterium or virus is used to refer to a bacterial or viral host cell carrying non-native genetic material, e.g., an immune modulator, such that the non-native genetic material is retained, expressed, and propagated. The stable bacterium or virus is capable of survival and/or growth in vitro, e.g., in medium, and/or in vivo, e.g., in hypoxic and/or necrotic tissues. For example, the stable bacterium or virus may be a genetically engineered bacterium comprising non-native genetic material encoding an immune modulator, in which the plasmid or chromosome carrying the non-native genetic material is stably maintained in the bacterium or virus, such that the immune modulator can be expressed in the bacterium or virus, and the bacterium or virus is capable of survival and/or growth in vitro and/or in vivo.

As used herein, the terms “modulate” and “treat” and their cognates refer to an amelioration of a cancer, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a cancer, either physically (e.g., stabilization of a discernible symptom), physiologically (e.g., stabilization of a physical parameter), or both. In another embodiment, “modulate” and “treat” refer to slowing the progression or reversing the progression of a cancer. As used herein, “prevent” and its cognates refer to delaying the onset or reducing the risk of acquiring a given cancer.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor, and does not necessarily encompass the elimination of the underlying cause of the cancer, e.g., an underlying genetic predisposition.

As used herein, the term “conventional cancer treatment” or “conventional cancer therapy” refers to treatment or therapy that is widely accepted and used by most healthcare professionals. It is different from alternative or complementary therapies, which are not as widely used. Examples of conventional treatment for cancer include surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion.

As used herein a “pharmaceutical composition” refers to a preparation of genetically engineered microorganism of the disclosure with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., a cancer. A therapeutically effective amount may, for example, be sufficient to treat, prevent, reduce the severity, delay the onset, and/or reduce the risk of occurrence of one or more symptoms of a disorder associated with cancerous cells. A therapeutically effective amount, as well as a therapeutically effective frequency of administration, can be determined by methods known in the art and discussed below.

In some embodiments, the term “therapeutic molecule” refers to a molecule or a compound that is results in prevention, delay of onset of symptoms, or amelioration of symptoms of a condition, e.g., a cancer. In some embodiments, a therapeutic molecule may be, for example, a cytokine, a chemokine, a single chain antibody, a ligand, a metabolic converter, e.g., arginine, a kynurnenine consumer, or an adenosine consumer, a T cell co-stimulatory receptor, a T cell co-stimulatory receptor ligand, an engineered chemotherapy, or a lytic peptide, among others.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

In one embodiment, the modified microorganism may be a bacterium, e.g., a genetically engineered bacterium. The modified microorganism, or genetically engineered microorganisms, such as the modified bacterium of the disclosure is capable of local and tumor-specific delivery of effectors and/or immune modulators, thereby reducing the systemic cytotoxicity and/or immune dysfunction associated with systemic administration of said molecules. The engineered bacteria may be administered systemically, orally, locally and/or intratumorally. In some embodiments, the genetically engineered bacteria are capable of targeting cancerous cells, particularly in the hypoxic regions of a tumor, and producing an effector molecule, e.g., an immune modulator, e.g., immune stimulator or sustainer provided herein. In some embodiments, the genetically engineered bacterium is bacterium that expresses an effector, e.g., immune modulator, under the control of a promoter that is activated by low-oxygen conditions, e.g., the hypoxic environment of a tumor.

In some embodiments, the tumor-targeting microorganism is a bacterium that is naturally capable of directing itself to cancerous cells, necrotic tissues, and/or hypoxic tissues. For example, bacterial colonization of tumors may be achieved without any specific genetic modifications in the bacteria or in the host (Yu et al., 2008). In some embodiments, the tumor-targeting bacterium is a bacterium that is not naturally capable of directing itself to cancerous cells, necrotic tissues, and/or hypoxic tissues, but is genetically engineered to do so. In some embodiments, the genetically engineered bacteria spread hematogenously to reach the targeted tumor(s). Bacterial infection has been linked to tumor regression (Hall, 1998; Nauts and McLaren, 1990), and certain bacterial species have been shown to localize to and lyse necrotic mammalian tumors (Jain and Forbes, 2001). Non-limiting examples of tumor-targeting bacteria are shown in Table 3.

TABLE 3
Bacteria with tumor-targeting capability

| Bacterial Strain | See, e.g., |
|---|---|
| Clostridium novyi-NT | Forbes, Neil S. “Profile of a |
| | bacterial tumor killer.” Nature |
| | biotechnology 24.12 (2006): |
| | 1484-1485. |
| Bifidobacterium spp | Liu, Sai, et al. “Tumor-targeting |
| Streptococcus spp | bacterial therapy: A potential treatment |
| Caulobacter spp | for oral cancer.” Oncology letters |
| Clostridium spp | 8.6 (2014): 2359-2366. |
| Escherichia coli MG1655 | Cronin, Michelle, et al. “High |
| Escherichia coli Nissle | resolution in vivo bioluminescent |
| Bifidobacterium breve | imaging for the study of bacterial |
| UCC2003 | tumour targeting.” PloS one 7.1 |
| Salmonella typhimurium | (2012): e30940.; Zhou, et al., Med |
| | Hypotheses. 2011 April; 76(4): |
| | 533-4. doi: 10.1016/j.mehy.2010.12.010. |
| | Epub 2011 Jan. 21; Zhang et al., |
| | Appl Environ Microbiol. 2012 |
| | November; 78(21): 7603-7610; |
| | Danino et al., Science Translational |
| | Medicine, 2015 Vol 7 |
| | Issue 289, pp. 289ra84 |
| Clostridium novyi-NT | Bernardes, Nuno, Ananda M. |
| Bifidobacterium spp | Chakrabarty, and Arsenio M. |
| Mycobacterium bovis | Fialho. “Engineering of bacterial |
| Listeria monocytogenes | strains and their products for |
| Escherichia coli | cancer therapy.” Applied |
| Salmonella spp | microbiology and biotechnology |
| Salmonella typhimurium | 97.12 (2013): 5189-5199. |
| Salmonella choleraesuis | Patyar, S., et al. “Bacteria in |
| Vibrio cholera | cancer therapy: a novel |
| Listeria monocytogenes | experimental strategy.” |
| Escherichia coli | J Biomed Sci 17.1 (2010): 21-30. |
| Bifidobacterium adolescentis | |
| Clostridium acetobutylicum | |
| Salmonella typhimurium | |
| Clostridium histolyticum | |
| Escherichia coli Nissle 1917 | Danino et al. “Programmable |
| | probiotics for detection of cancer in |
| | urine.” Sci Transl Med. 2015 May |
| | 27; 7(289): 289ra84 |

In some embodiments, the gene of interest is expressed in a bacterium which enhances the efficacy of immunotherapy. Recent studies have suggested that the presence of certain types of gut microbes in mice can enhance the anti-tumor effects of cancer immunotherapy without increasing toxic side effects (M. Vétizou et al., “Anticancer immunotherapy by CTLA-4 blockade relies on the gut microbiota,” Science, doi:10.1126/aad1329, 2015; A. Sivan et al., “Commensal Bifidobacterium promotes antitumor immunity and facilitates anti-PD-L1 efficacy,” Science, doi:0.1126/science.aac4255, 2015). Whether the gut microbial species identified in these mouse studies will have the same effect in people is not clear. Vetizou et al (2015) describe T cell responses specific for Bacteroides thetaiotaomicron or Bacteroides fragilis that were associated with the efficacy of CTLA-4 blockade in mice and in patients. Sivan et al. (2015) illustrate the importance of Bifidobacterium to antitumor immunity and anti-PD-L1 antibody against (PD-1 ligand) efficacy in a mouse model of melanoma. In some embodiments, the bacteria expressing the one or more immune modulators are Bacteroides. In some embodiments, the bacteria expressing the one or more immune modulators are Bifidobacterium. In some embodiments, the bacteria expressing the one or more immune modulators are Escherichia Coli Nissle. In some embodiments, the bacteria expressing the one or more immune modulators are Clostridium novyi-NT. In some embodiments, the bacteria expressing the one or more immune modulators are Clostridium butyricum miyairi.

In certain embodiments, the modified microorganisms or genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, Vibrio cholera, and the bacteria shown in Table 3. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis. In some embodiments, Lactobacillus is used for tumor-specific delivery of one or more immune modulators. Lactobacillus casei injected intravenously has been found to accumulate in tumors, which was enhanced through nitroglycerin (NG), a commonly used NO donor, likely due to the role of NO in increasing the blood flow to hypovascular tumors (Fang et al., 2016 (Methods Mol Biol. 2016; 1409:9-23. Enhancement of Tumor-Targeted Delivery of Bacteria with Nitroglycerin Involving Augmentation of the EPR Effect).

In some embodiments, the genetically engineered bacteria are obligate anaerobes. In some embodiments, the genetically engineered bacteria are Clostridia and capable of tumor-specific delivery of immune modulators. Clostridia are obligate anaerobic bacterium that produce spores and are naturally capable of colonizing and in some cases lysing hypoxic tumors (Groot et al., 2007). In experimental models, Clostridia have been used to deliver pro-drug converting enzymes and enhance radiotherapy (Groot et al., 2007). In some embodiments, the genetically engineered bacteria is selected from the group consisting of Clostridium novyi-NT, Clostridium histolyticium, Clostridium tetani, Clostridium oncolyticum, Clostridium sporogenes, and Clostridium beijerinckii (Liu et al., 2014). In some embodiments, the Clostridium is naturally non-pathogenic. For example, Clostridium oncolyticum is a pathogenic and capable of lysing tumor cells. In alternate embodiments, the Clostridium is naturally pathogenic but modified to reduce or eliminate pathogenicity. For example, Clostridium novyi are naturally pathogenic, and Clostridium novyi-NT are modified to remove lethal toxins. Clostridium novyi-NT and Clostridium sporogenes have been used to deliver single-chain HIF-1α antibodies to treat cancer and is an “excellent tumor colonizing Clostridium strains” (Groot et al., 2007).

In some embodiments, the genetically engineered bacteria facultative anaerobes. In some embodiments, the genetically engineered bacteria are Salmonella, e.g., Salmonella typhimurium, and are capable of tumor-specific delivery of immune modulators. Salmonella are non-spore-forming Gram-negative bacteria that are facultative anaerobes. In some embodiments, the Salmonella are naturally pathogenic but modified to reduce or eliminate pathogenicity. For example, Salmonella typhimurium is modified to remove pathogenic sites (attenuated). In some embodiments, the genetically engineered bacteria are Bifidobacterium and capable of tumor-specific delivery of immune modulators. Bifidobacterium are Gram-positive, branched anaerobic bacteria. In some embodiments, the Bifidobacterium is naturally non-pathogenic. In alternate embodiments, the Bifidobacterium is naturally pathogenic but modified to reduce or eliminate pathogenicity. Bifidobacterium and Salmonella have been shown to preferentially target and replicate in the hypoxic and necrotic regions of tumors (Yu et al., 2014).

In some embodiments, the genetically engineered bacteria are Gram-negative bacteria. In some embodiments, the genetically engineered bacteria are E. coli. For example, E. coli Nissle has been shown to preferentially colonize tumor tissue in vivo following either oral or intravenous administration (Zhang et al., 2012 and Danino et al., 2015). E. coli have also been shown to exhibit robust tumor-specific replication (Yu et al., 2008). In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added).

The genetically engineered bacteria of the invention may be destroyed, e.g., by defense factors in tissues or blood serum (Sonnenborn et al., 2009). In some embodiments, the genetically engineered bacteria are administered repeatedly. In some embodiments, the genetically engineered bacteria are administered once.

In certain embodiments, the effectors and/or immune modulator(s) described herein are expressed in one species, strain, or subtype of genetically engineered bacteria. In alternate embodiments, the effector and/or immune modulator is expressed in two or more species, strains, and/or subtypes of genetically engineered bacteria. One of ordinary skill in the art would appreciate that the genetic modifications disclosed herein may be modified and adapted for other species, strains, and subtypes of bacteria.

Further examples of bacteria which are suitable are described in International Patent Publication WO/2014/043593, the contents of which is herein incorporated by reference in its entirety. In some embodiments, such bacteria are mutated to attenuate one or more virulence factors.

In some embodiments, the genetically engineered bacteria of the disclosure proliferate and colonize a tumor. In some embodiments, colonization persists for several days, several weeks, several months, several years or indefinitely. In some embodiments, the genetically engineered bacteria do not proliferate in the tumor and bacterial counts drop off quickly post injection, e.g., less than a week post injection, until no longer detectable.

### Bacteriophages

In some embodiments, the genetically engineered bacteria of the disclosure comprise one or more lysogenic, dormant, temperate, intact, defective, cryptic, or satellite phage or bacteriocins/phage tail or gene transfer agents in their natural state. In some embodiments, the prophage or bacteriophage exists in all isolates of a particular bacterium of interest. In some embodiments, the bacteria are genetically engineered derivatives of a parental strain comprising one or more of such bacteriophage. In any of the embodiments described herein, the bacteria may comprise one or more modifications or mutations within a prophage or bacteriophage genome which alters the properties or behavior of the bacteriophage. In some embodiments, the modifications or mutations prevent the prophage from entering or completing the lytic process. In some embodiments, the modifications or mutations prevent the phage from infecting other bacteria of the same or a different type. In some embodiments, the modifications or mutations alter the fitness of the bacterial host. In some embodiments, the modifications or mutations no not alter the fitness of the bacterial host. In some embodiments, the modifications or mutations have an impact on the desired effector function, e.g., on levels of expression of the effector molecule, e.g., immune modulator, e.g., immune stimulator or sustainer, of the genetically engineered bacterium. In some embodiments, the modifications or mutations have no impact on the desired function e.g., on levels of expression of the effector molecule or on levels of activity of the effector molecule.

Phage genome size varies, ranging from the smallest Leuconostoc phage L5 (2,435 bp), ˜11.5 kbp (e.g. Mycoplasma phage P1), ˜21kbp (e.g. Lactococcus phage c2), and ˜30 kbp (e.g. Pasteurella phage F108) to the almost 500 kbp genome of Bacillus megaterium phage G (Hatfull and Hendrix; Bacteriophages and their Genomes, Curr Opin Virol. 2011 Oct. 1; 1(4): 298-303, and references therein). Phage genomes may encode less than 10 genes up to several hundreds of genes. Temperate phages or prophages are typically integrated into the chromosome(s) of the bacterial host, although some examples of phages that are integrated into bacterial plasmids also exist (Little, Loysogeny, Prophage Induction, and Lysogenic Conversion. In: Waldor M K, Friedman D I, Adhya S, editors. Phages Their Role in Bacterial Pathogenesis and Biotechnology. Washington D.C.: ASM Press; 2005. pp. 37-54). In some cases, the phages are always located at the same position within the bacterial host chromosome(s), and this position is specific to each phage, i.e., different phages are located at different positions. Other phages can integrate at numerous different locations.

Accordingly, the bacteria of the disclosure comprise one or more phages genomes which may vary in length, from at least about 1 bp to 10 kb, from at least about 10 kb to 20 kb, from at least about 20 kb to 30 kb, from at least about 30 kb to 40 kb, from at least about 30 kb to 40 kb, from at least about 40 kb to 50 kb, from at least about 50 kb to 60 kb, from at least about 60 kb to 70 kb, from at least about 70 kb to 80 kb, from at least about 80 kb to 90 kb, from at least about 90 kb to 100 kb, from at least about 100 kb to 120 kb, from at least about 120 kb to 140 kb, from at least about 140 kb to 160 kb, from at least about 160 kb to 180 kb, from at least about 180 kb to 200 kb, from at least about 200 kb to 180 kb, from at least about 160 kb to 250 kb, from at least about 250 kb to 300 kb, from at least about 300 kb to 350 kb, from at least about 350 kb to 400 kb, from at least about 400 kb to 500 kb, from at least about 500 kb to 1000 kb. In one embodiment, the genetically engineered bacteria comprise a bacteriophage genome greater than 1000 kb in length.

In some embodiments, the bacteria of the disclosure comprise one or more phages genomes, which comprise one or more genes encoding one or more polypeptides. In one embodiment, the genetically engineered bacteria comprise a bacteriophage genome comprising at least about 1 to 5 genes, at least about 5 to 10 genes, at least about 10 to 15 genes, at least about 15 to 20 genes, at least about 20 to 25 genes, at least about 25 to 30 genes, at least about 30 to 35 genes, at least about 35 to 40 genes, at least about 40 to 45 genes, at least about 45 to 50 genes, at least about 50 to 55 genes, at least about 55 to 60 genes, at least about 60 to 65 genes, at least about 65 to 70 genes, at least about 70 to 75 genes, at least about 75 to 80 genes, at least about 80 to 85 genes, at least about 85 to 90 genes, at least about 90 to 95 genes, at least about 95 to 100 genes, at least about 100 to 115 genes, at least about 115 to 120 genes, at least about 120 to 125 genes, at least about 125 to 130 genes, at least about 130 to 135 genes, at least about 135 to 140 genes, at least about 140 to 145 genes, at least about 145 to 150 genes, at least about 150 to 160 genes, at least about 160 to 170 genes, at least about 170 to 180 genes, at least about 180 to 190 genes, at least about 190 to 200 genes, at least about 200 to 300 genes. In one embodiment, the genetically engineered bacteria comprise a bacteriophage genome comprising more than about 300 genes.

In some embodiments, the phage is always or almost always located at the same location or position within the bacterial host chromosome(s) in a particular species. In some embodiments, the phages are found integrated at different locations within the host chromosome in a particular species. In some embodiments, the phage is located on a plasmid.

In some embodiments, the prophage may be a defective or a cryptic prophage. Defective prophages can no longer undergo a lytic cycle. Cryptic prophages may not be able to undergo a lytic cycle or never have undergone a lytic cycle (Bobay et al., 2014). In some embodiments, the bacteria comprise one or more satellite phage genomes. Satellite phages are otherwise functional phages that do not carry their own structural protein genes, and have genomes that are configures for encapsulation by the structural proteins of other specific phages (Six and Klug Bacteriophage P4: a satellite virus depending on a helper such as prophage P2, Virology, Volume 51, Issue 2, February 1973, Pages 327-344).

In some embodiments, the bacteria comprise one or more tailiocins. Many bacteria, both gram positive and gram negative, produce a variety of particles resembling phage tails that are functional without an associated phage head (termed tailiocins), and many of which have been shown to have bacteriocin properties (reviewed in Ghequire and Mot, The Tailocin Tale: Peeling off Phage; Trends in Microbiology, October 2015, Vol. 23, No. 10). Phage tail-like bacteriocins are classified two different families: contractile phage tail-like (R-type) and noncontractile but flexible ones (F-type). In some embodiments, the bacteria comprise one or more gene transfer agents. Gene transfer agents (GTAs) are phage-like elements that are encoded by some bacterial genomes. Although GTAs resemble phages, they lack the hallmark capabilities that define typical phages, and they package random fragments of the host cell DNA and then transfer them horizontally to other bacteria of the same species (reviewed in Lang et al., Gene transfer agents: phage-like elements of genetic exchange, Nat Rev Microbiol. 2012 Jun. 11; 10(7): 472-482). There, the DNA can replace the resident cognate chromosomal region by homologous recombination. However, these particles cannot propagate as viruses, as the vast majority of the particles do not carry the genes that encode the GTA. In some embodiments, the bacteria comprise one or more filamentous virions. Filamentous virions integrate as dsDNA prophages (reviewed in Marvin D A, et al, Structure and assembly of filamentous bacteriophages, Prog Biophys Mol Biol. 2014 April; 114(2):80-122). In any of these embodiments, the bacteria described herein comprising defective or a cryptic prophage, satellite phage genomes, tailiocins, gene transfer agents, filamentous virions, which may comprise one or more modifications or mutations within their sequence.

Prophages can be either identified experimentally or computationally. The experimental approach involves inducing the host bacteria to release phage particles by exposing them to UV light or other DNA-damaging conditions. However, in some cases, the conditions under which a prophage is induced is unknown, and therefore the absence of plaques in a plaque assay does not necessarily prove the absence of a prophage. Additionally, this approach can show only the existence of viable phages, but will not reveal defective prophages. As such, computational identification of prophages from genomic sequence data has become the most preferred route.

Co-pending International Patent Application PCT/US18/38840, filed Jun. 21, 2018, herein incorporated by reference in their entireties, provide non-limiting examples of probiotic bacteria which contain number of potential bacteriophages contained in the bacterial genome as determined by Phaster scoring. Phaster scoring is described in detail at phaster.ca and in Zhou, et al. (“PHAST: A Fast Phage Search Tool” Nucl. Acids Res. (2011) 39(suppl 2): W347-W352) and Arndt et al. (Arndt, et al. (2016) PHASTER: a better, faster version of the PHAST phage search tool. Nucleic Acids Res., 2016 May 3). In brief, three methods are applied with different criteria to score for prophage regions (as intact, questionable, or incomplete) within a provided bacterial genome sequence.

In any of the embodiments described herein, the bacteria described herein may comprise one or more modifications or mutations within an existing prophage or bacteriophage genome. In some embodiments, these modifications alter the properties or behavior of the prophage. In some embodiments, the modifications or mutations prevent the prophage from entering or completing the lytic process. In some embodiments, the modifications or mutations prevent the phage from infecting other bacteria of the same or a different type. In some embodiments, the modifications or mutations alter the fitness of the bacterial host. In some embodiments, the modifications or mutations do not alter the fitness of the bacterial host. In some embodiments, the modifications or mutations have an impact on the desired effector function, e.g., of a genetically engineered bacterium. In some embodiments, the modifications or mutations do not have an impact on the desired effector function, e.g., of a genetically engineered bacterium.

In some embodiments, the modifications or mutations reduce entry or completion of prophage lytic process at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold. In some embodiments, the modifications or mutations completely prevent entry or completion of prophage lytic process.

In some embodiments, the modifications or mutations reduce entry or completion of prophage lytic process by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100%.

In some embodiments, the mutations include one or more deletions within the phage genome sequence. In some embodiments, the mutations include one or more insertions into the phage genome sequence. In some embodiments, an antibiotic cassette can be inserted into one or more positions within the phage genome sequence. In some embodiments, the mutations include one or more substitutions within the phage genome sequence. In some embodiments, the mutations include one or more inversions within the phage genome sequence. In some embodiments, the modifications within the phage genome are combinations of two or more of insertions, deletions, substitutions, or inversions within one or more phage genome genes. In any of the embodiments described herein, the modifications may result in one or more frameshift mutations in one or more genes within the phage genome.

An any of these embodiments, the mutations can be located within or encompass one or more genes encoding proteins of various functions, e.g., lysis, e.g., proteases or lysins, toxins, antibiotic resistance, translation, structural (e.g., head, tail, collar, or coat proteins)., bacteriophage assembly, recombination (e.g., integrases, invertases, or transposases), or replication (e.g., primases, tRNA related proteins), phage insertion, attachment, packaging, or terminases.

In some embodiments, described herein genetically engineered bacteria are engineered Escherichia coli strain Nissle 1917 (E. coli Nissle). As described in co-pending International Patent Application PCT/US18/38840, filed Jun. 21, 2018, herein incorporated by reference in their entireties, in more detail herein in the examples, routine testing procedures identified bacteriophage production from Escherichia coli Nissle 1917 (E. coli Nissle) and related engineered derivatives. To determine the source of the bacteriophage, a collaborative bioinformatics assessment of the genomes of E. coli Nissle, and engineered derivatives was conducted to analyze genomic sequences of the strains for evidence of prophages, to assess any identified prophage elements for the likelihood of producing functional phage, to compare any functional phage elements with other known phage identified among bacterial genomic sequences, and to evaluate the frequency with which prophage elements are found in other sequenced Escherichia coli (E. coli) genomes. The assessment tools included phage prediction software (PHAST and PHASTER), SPAdes genome assembler software, software for mapping low-divergent sequences against a large reference genome (BWA MEM), genome sequence alignment software (MUMmer), and the National Center for Biotechnology Information (NCBI) nonredundant database. The assessment results showed that E. coli Nissle and engineered derivatives analyzed contain three candidate prophage elements, with two of the three (Phage 2 and Phage 3) containing most genetic features characteristic of intact phage genomes. Two other possible phage elements were also identified. Of note, the engineered strains did not contain any additional phage elements that were not identified in parental E. coli Nissle, indicating that plaque-forming units produced by these strains originate from one of these endogenous phages (Phage 3). Interestingly, Phage 3 is unique to E. coli Nissle among a collection of almost 6000 sequenced E. coli genomes, although related sequences limited to short regions of homology with other putative prophage elements are found in a small number of genomes. Phage 3, but not any of the other Phage, was found to be inducible and result in bacterial lysis upon induction.

Prophages are very common among E. coli strains, with E. coli Nissle containing a relatively small number of prophage sequences compared to the average number found in a well-characterized set of sequenced E. coli genomes. As such, prophage presence in the engineered strains is part of the natural state of this species and the prophage features of the engineered strains analyzed were consistent with the progenitor strain, E. coli Nissle.

In some embodiments, the bacteria described herein may comprise one or more modifications or mutations within the E. coli Nissle Phage 3 genome which alters the properties or behavior of Phage 3. In some embodiments, the modifications or mutations prevent Phage 3 from entering or completing the lytic process. In some embodiments, the modifications or mutations prevent the E. coli Nissle Phage 3 from infecting other bacteria of the same or a different type. In some embodiments, the modifications or mutations improve the fitness of the bacterial host. In some embodiments, the no effect fitness of the bacterial host is observed. In some embodiments, the modifications or mutations have an impact on the desired effector function, e.g., expression of the immune modulator. In some embodiments, no impact on the desired effector function, e.g., expression of the immune modulator, is observed.

In some embodiments, the mutations introduced into the bacterial chassis include one or more deletions within the E. coli Nissle Phage 3 genome sequence. In some embodiments, the mutations include one or more insertions into the E. coli Nissle Phage 3 genome sequence. In some embodiments, an antibiotic cassette can be inserted into one or more positions within the E. coli Nissle Phage 3 genome sequence. Mutations withing Phage 3 are described in more details in Co-pending U.S. provisional applications 62/523,202 and 62/552,829, herein incorporated by reference in their entireties.

TABLE 4
E. coli Nissle Phage 3 Genome

| | | | | GI | | | SEQ | SEQ |
|---|---|---|---|---|---|---|---|---|
| Description | Position | Length | Orientation | Number | Protein ID | Product | ID NO | ID NO |
| ECOLIN_09965 | 27 . . . 998 | 972 | <= | 660511998 | AID78889.1 | lipid A biosynthesis | 1286 | 1359 |
| | | | | | | (KDO)2-(lauroyl)- | | |
| | | | | | | lipid IVA | | |
| | | | | | | acyltransferase | | |
| ECOLIN_09970 | 1117 . . . 2439 | 1323 | <= | 660511999 | AID78890.1 | peptidase | 1287 | 1360 |
| ECOLIN_09975 | 2455 . . . 3387 | 933 | <= | 660512000 | AID78891.1 | zinc ABC transporter | 1288 | 1361 |
| | | | | | | substrate-binding | | |
| | | | | | | protein | | |
| ECOLIN_09980 | 3466 . . . 4221 | 756 | => | 660512001 | AID78892.1 | zinc ABC transporter | 1289 | 1362 |
| | | | | | | ATPase | | |
| ECOLIN_09985 | 4218 . . . 5003 | 786 | => | 660512002 | AID78893.1 | high-affinity zinc | 1290 | 1363 |
| | | | | | | transporter membrane | | |
| | | | | | | component | | |
| ECOLIN_09990 | 5150 . . . 6160 | 1011 | <= | 660512003 | AID78894.1 | ATP-dependent DNA | 1291 | 1364 |
| | | | | | | helicase RuvB | | |
| ECOLIN_09995 | 6169 . . . 6780 | 612 | <= | 660512004 | AID78895.1 | ATP-dependent DNA | 1292 | 1365 |
| | | | | | | helicase RuvA | | |
| ECOLIN_10000 | 7056 . . . 7658 | 603 | => | 660512005 | AID78896.1 | hypothetical protein | 1293 | 1366 |
| ECOLIN_10005 | 7660 . . . 8181 | 522 | <= | 660512006 | AID78897.1 | Holliday junction | 1294 | 1367 |
| | | | | | | resolvase | | |
| ECOLIN_10010 | 8216 . . . 8956 | 741 | <= | 660512007 | AID78898.1 | hypothetical protein | 1295 | 1368 |
| ECOLIN_10015 | 8985 . . . 9428 | 444 | <= | 660512008 | AID78899.1 | dihydroneopterin | 1296 | 1369 |
| | | | | | | triphosphate | | |
| | | | | | | pyrophosphatase | | |
| ECOLIN_10020 | 9430 . . . 11,202 | 1773 | <= | 660512009 | AID78900.1 | aspartyl-tRNA | 1297 | 1370 |
| | | | | | | synthetase | | |
| ECOLIN_10025 | 11,512 . . . 12,078 | 567 | => | 660512010 | AID78901.1 | hydrolase | 1298 | 1371 |
| ECOLIN_10030 | 12,680 . . . 13,069 | 390 | <= | 660512011 | AID78902.1 | DNA polymerase V | 1299 | 1372 |
| | | | | | | ECOLIN_10030 | | |
| ECOLIN_10035 | 13,148 . . . 13,390 | 243 | => | 660512012 | AID78903.1 | MsgA | 1300 | 1373 |
| ECOLIN_10040 | 13,426 . . . 13,806 | 381 | => | 660512013 | AID78904.1 | hypothetical protein | 1301 | 1374 |
| ECOLIN_10045 | 13,808 . . . 14,251 | 444 | => | 660512014 | AID78905.1 | hypothetical protein | 1302 | 1375 |
| ECOLIN_10050 | 14,223 . . . 14,816 | 594 | <= | 660512015 | AID78906.1 | phage tail protein | 1303 | 1376 |
| ECOLIN_10055 | 14,816 . . . 15,748 | 933 | <= | 660512016 | AID78907.1 | tail protein | 1304 | 1377 |
| ECOLIN_10065 | 16,519 . . . 20,445 | 3927 | <= | 660512017 | AID78908.1 | host specificity | 1305 | 1378 |
| | | | | | | protein | | |
| ECOLIN_10070 | 20,488 . . . 21,105 | 618 | <= | 660512018 | AID78909.1 | tail protein | 1306 | 1379 |
| ECOLIN_10075 | 21,098 . . . 21,817 | 720 | <= | 660512019 | AID78910.1 | peptidase P60 | 1307 | 1380 |
| ECOLIN_10080 | 21,820 . . . 22,557 | 738 | <= | 660512020 | AID78911.1 | hypothetical protein | 1308 | 1381 |
| ECOLIN_10085 | 22,614 . . . 22,952 | 339 | <= | 660512021 | AID78912.1 | tail protein | 1309 | 1382 |
| ECOLIN_10090 | 22,949 . . . 26,086 | 3138 | <= | 660512022 | AID78913.1 | tail protein | 1310 | 1383 |
| ECOLIN_10095 | 26,070 . . . 26,342 | 273 | <= | 660512023 | AID78914.1 | tail protein | 1311 | 1384 |
| ECOLIN_10100 | 26,393 . . . 26,824 | 432 | <= | 660512024 | AID78915.1 | tail protein | 1312 | 1385 |
| ECOLIN_10105 | 26,835 . . . 27,578 | 744 | <= | 660512025 | AID78916.1 | tail fiber protein | 1313 | 1386 |
| ECOLIN_10110 | 27,588 . . . 27,989 | 402 | <= | 660512026 | AID78917.1 | Minor tail protein U | 1314 | 1387 |
| ECOLIN_10115 | 27,986 . . . 28,558 | 573 | <= | 660512027 | AID78918.1 | tail protein | 1315 | 1388 |
| ECOLIN_10120 | 28,574 . . . 28,816 | 243 | <= | 660512028 | AID78919.1 | DNA breaking- | 1316 | 1389 |
| | | | | | | rejoining protein | | |
| ECOLIN_10125 | 28,842 . . . 29,168 | 327 | <= | 660512029 | AID78920.1 | hypothetical protein | 1317 | 1390 |
| ECOLIN_10130 | 29,251 . . . 31,197 | 1947 | <= | 660512030 | AID78921.1 | peptidase S14 | 1318 | 1391 |
| ECOLIN_10135 | 31,211 . . . 32,710 | 1500 | <= | 660512031 | AID78922.1 | capsid protein | 1319 | 1392 |
| ECOLIN_10140 | 32,707 . . . 32,922 | 216 | <= | 660512032 | AID78923.1 | hypothetical protein | 1320 | 1393 |
| ECOLIN_10145 | 32,919 . . . 35,021 | 2103 | <= | 660512033 | AID78924.1 | DNA packaging | 1321 | 1394 |
| | | | | | | protein | | |
| ECOLIN_10150 | 35,021 . . . 35,509 | 489 | <= | 660512034 | AID78925.1 | terminase | 1322 | 1395 |
| ECOLIN_10160 | 35,693 . . . 36,421 | 729 | <= | 660512035 | AID78926.1 | hypothetical protein | 1323 | 1396 |
| ECOLIN_10165 | 36,596 . . . 36,826 | 231 | <= | 660512036 | AID78927.1 | hypothetical protein | 1324 | 1397 |
| ECOLIN_10170 | 36,825 . . . 37,421 | 597 | => | 660512037 | AID78928.1 | hypothetical protein | 1325 | 1398 |
| ECOLIN_10175 | 37,490 . . . 37,687 | 198 | <= | 660512038 | AID78929.1 | hypothetical protein | 1326 | 1399 |
| ECOLIN_10180 | 37,901 . . . 38,380 | 480 | <= | 660512039 | AID78930.1 | hypothetical protein | 1327 | 1400 |
| ECOLIN_10185 | 38,401 . . . 38,949 | 549 | <= | 660512040 | AID78931.1 | lysozyme | 1328 | 1401 |
| ECOLIN_10190 | 38,921 . . . 39,199 | 279 | <= | 660512041 | AID78932.1 | holin | 1329 | 1402 |
| ECOLIN_10195 | 39,345 . . . 40,397 | 1053 | <= | 660512042 | AID78933.1 | DNA adenine | 1330 | 1403 |
| | | | | | | methylase | | |
| ECOLIN_10200 | 40,548 . . . 40,739 | 192 | <= | 660512043 | AID78934.1 | hypothetical protein | 1331 | 1404 |
| ECOLIN_10205 | 40,908 . . . 41,807 | 900 | <= | 660512044 | AID78935.1 | serine protease | 1332 | 1405 |
| ECOLIN_10210 | 41,820 . . . 42,026 | 207 | <= | 660512045 | AID78936.1 | hypothetical protein | 1333 | 1406 |
| ECOLIN_10220 | 42,459 . . . 43,148 | 690 | <= | 660512046 | AID78937.1 | antitermination | 1334 | 1407 |
| | | | | | | protein | | |
| ECOLIN_10225 | 43,170 . . . 44,165 | 996 | <= | 660512047 | AID78938.1 | hypothetical protein | 1335 | 1408 |
| ECOLIN_10230 | 44,162 . . . 44,845 | 684 | <= | 660512048 | AID78939.1 | antirepressor | 1336 | 1409 |
| ECOLIN_10235 | 44,859 . . . 45,245 | 387 | <= | 660512049 | AID78940.1 | crossover junction | 1337 | 1410 |
| | | | | | | endodeoxyribonuclease | | |
| ECOLIN_10240 | 45,242 . . . 46,561 | 1320 | <= | 660512050 | AID78941.1 | adenine | 1338 | 1411 |
| | | | | | | methyltransferase, | | |
| | | | | | | DNA | | |
| | | | | | | methyltransferase | | |
| | | | | | | ECOLIN_10240 | | |
| ECOLIN_10245 | 46,558 . . . 47,439 | 882 | <= | 660512051 | AID78942.1 | GntR family | 1339 | 1412 |
| | | | | | | transcriptional | | |
| | | | | | | regulator | | |
| | | | | | | ECOLIN_10245 | | |
| ECOLIN_10250 | 47,449 . . . 47,787 | 339 | <= | 660512052 | AID78943.1 | hypothetical protein | 1340 | 1413 |
| ECOLIN_10255 | 47,784 . . . 48,347 | 564 | <= | 660512053 | AID78944.1 | hypothetical protein, | 1341 | 1414 |
| | | | | | | completely unknown | | |
| ECOLIN_10260 | 48,379 . . . 48,636 | 258 | <= | 660512054 | AID78945.1 | hypothetical protein, | 1342 | 1415 |
| | | | | | | cI repressor | | |
| | | | | | | ECOLIN_10260 | | |
| ECOLIN_10265 | 48,715 . . . 49,425 | 711 | => | 660512055 | AID78946.1 | hypothetical protein, | 1343 | 1416 |
| | | | | | | Domain of unknown | | |
| | | | | | | function (DUF4222); | | |
| | | | | | | This short protein is | | |
| | | | | | | likely to be of phage | | |
| | | | | | | origin. For example it | | |
| | | | | | | is found in | | |
| | | | | | | Enterobacteria phage | | |
| | | | | | | YYZ-2008. It is | | |
| | | | | | | largely found in | | |
| | | | | | | enteric bacteria. The | | |
| | | | | | | molecular function of | | |
| | | | | | | this protein is | | |
| | | | | | | unknown. | | |
| ECOLIN_10270 | 49,868 . . . 50,065 | 198 | <= | 660512056 | AID78947.1 | hypothetical protein | 1344 | 1417 |
| ECOLIN_10275 | 50,378 . . . 51,295 | 918 | => | 660512057 | AID78948.1 | DNA recombinase In | 1345 | 1418 |
| | | | | | | Escherichia coli, | | |
| | | | | | | RdgC is required for | | |
| | | | | | | growth in | | |
| | | | | | | recombination- | | |
| | | | | | | deficient | | |
| | | | | | | exonuclease-depleted | | |
| | | | | | | strains. Under these | | |
| | | | | | | conditions, RdgC | | |
| | | | | | | may act as an | | |
| | | | | | | exonuclease to | | |
| | | | | | | remove collapsed | | |
| | | | | | | replication forks, in | | |
| | | | | | | the absence of the | | |
| | | | | | | normal repair | | |
| | | | | | | mechanisms | | |
| | | | | | | ECOLIN_10275 | | |
| ECOLIN_10280 | 51,404 . . . 51,943 | 540 | => | 660512058 | AID78949.1 | hypothetical protein, | 1346 | 1419 |
| | | | | | | 5′ Deoxynucleotidase | | |
| | | | | | | YfbR and HD | | |
| | | | | | | superfamily | | |
| | | | | | | hydrolases | | |
| | | | | | | ECOLIN_10280 | | |
| ECOLIN_10290 | 52,104 . . . 52,358 | 255 | => | 660512059 | AID78950.1 | hypothetical protein | 1347 | 1420 |
| | | | | | | Multiple Antibiotic | | |
| | | | | | | Resistance Regulator | | |
| | | | | | | (MarR) family of | | |
| | | | | | | transcriptional | | |
| | | | | | | regulators | | |
| ECOLIN_10295 | 52,355 . . . 52,702 | 348 | => | 660512060 | AID78951.1 | hypothetical protein, | 1348 | 1421 |
| | | | | | | unknown ead like | | |
| | | | | | | protein in P22 | | |
| ECOLIN_10300 | 52,704 . . . 53,012 | 309 | => | 660512061 | AID78952.1 | hypothetical protein, | 1349 | 1422 |
| | | | | | | totally unknown | | |
| ECOLIN_10305 | 53,026 . . . 53,493 | 468 | => | 660512062 | AID78953.1 | hypothetical protein, | 1350 | 1423 |
| | | | | | | Protein of unknown | | |
| | | | | | | function (DUF550); | | |
| | | | | | | This family is found | | |
| | | | | | | in a range of | | |
| | | | | | | Proteobacteria and a | | |
| | | | | | | few P-22 dsDNA | | |
| | | | | | | virus particles. The | | |
| | | | | | | function is currently | | |
| | | | | | | not known. Similar to | | |
| | | | | | | P22 EA gene | | |
| | | | | | | ECOLIN_10305 | | |
| ECOLIN_10310 | 53,496 . . . 53,750 | 255 | => | 660512063 | AID78954.1 | hypothetical protein, | 1351 | 1424 |
| | | | | | | Phage repressor | | |
| | | | | | | protein C, contains | | |
| | | | | | | Cro/C1-type HTH | | |
| | | | | | | and peptisase s24 | | |
| | | | | | | domains | | |
| ECOLIN_10315 | 53,772 . . . 54,341 | 570 | => | 660512064 | AID78955.1 | hypothetical protein, | 1352 | 1425 |
| | | | | | | 3′-5′ exonuclease | | |
| | | | | | | ECOLIN_10315 | | |
| ECOLIN_10320 | 54,382 . . . 54,618 | 237 | => | 660512065 | AID78956.1 | excisionase | 1353 | 1426 |
| | | | | | | ECOLIN_10320 | | |
| ECOLIN_10325 | 54,677 . . . 55,990 | 1314 | => | 660512066 | AID78957.1 | integrase, Phage | 1354 | 1427 |
| | | | | | | integrase family; | | |
| | | | | | | Members of this | | |
| | | | | | | family cleave DNA | | |
| | | | | | | substrates by a series | | |
| | | | | | | of staggered XerC | | |
| ECOLIN_10330 | 56,017 . . . 56,742 | 726 | => | 660512067 | AID78958.1 | hypothetical protein | 1355 | 1428 |
| ECOLIN_10335 | 56,795 . . . 57,190 | 396 | => | 660512068 | AID78959.1 | membrane protein | 1356 | 1429 |
| ECOLIN_10340 | 57,231 . . . 57,974 | 744 | => | 660512069 | AID78960.1 | tRNA | 1357 | 1430 |
| | | | | | | methyltransferase | | |
| ECOLIN_10345 | 57,971 . . . 58,942 | 972 | => | 660512070 | AID78961.1 | tRNA | 1358 | 1431 |
| | | | | | | methyltransferase | | |

In one specific embodiment, at least about 9000 to 10000 bp of the E. coli Nissle Phage 3 genome are mutated, e.g., in one example, 9687 bp of the E. coli Nissle Phage 3 genome are deleted.

In any of the embodiments described herein, the modifications encompass are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In one embodiment, the mutation is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the mutation is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the mutation is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a deletion mutation of ECOLIN_10175. In one embodiment, the phage genome mutation or deletion is located at one or more positions within SEQ ID NO: 1285. In some embodiments, at least about 0-1%, 1%-10%, 10% to 20%, 20% to 30%, 30% to 40%, 40% to 50%, 50% to 60%, 60% to 70%, 70% to 80%, 80% to 90% of SEQ ID NO: 1432 is deleted from the phage genome. In some embodiments, at least about 91%, at least about 92%, at least about 93%, at least about 94%, at least about 95%, at least about 96%, at least about 97%, at least about 98%, at least about 99% or at least about 100% of SEQ ID NO: 1432 is deleted from the phage genome. In some embodiments, at least about 99%, 98%, 97%, 96%, 95%, 94%, 93%, 92%, 91% or 90% of SEQ ID NO: 1432 is deleted from the phage genome. In one embodiment, a sequence comprising SEQ ID NO: 1432 is deleted from the phage 3 genome. In one embodiment, the sequence of SEQ ID NO: 1432 is deleted from the Phage 3 genome. In one embodiments, the genetically engineered bacteria comprise modified phage genome sequence comprising SEQ ID NO: 1433. In one embodiments, the genetically engineered bacteria comprise modified phage genome sequence consisting of SEQ ID NO: 1433.

### Effector Molecules

### Oncolysis and Activation of an Innate Immune Response

In certain embodiments, the effector molecule(s), or immune modulators(s) of the disclosure generates an innate antitumor immune response. In certain embodiments, the immune modulators(s) of the disclosure generates a local antitumor immune response. In some aspects, the effector molecule, or immune modulator, is able to activate systemic antitumor immunity against distant cancer cells. In certain embodiments, the immune modulators(s) generates a systemic or adaptive antitumor immune response. In some embodiments, the immune modulators(s) result in long-term immunological memory. Examples of suitable immune modulators(s), e.g., immune initiators and/or immune sustainers are described herein.

In some embodiments, one or more immune modulators may be produced by a modified microorganism described herein. In other embodiments, one or more immune modulators may be administered in combination with a modified microorganism capable of producing a second immune modulator(s). For example, one or more immune initiators may be administered in combination with a modified microorganism capable of producing one or more immune sustainers. In another embodiment, one or more immune sustainers may be administered in combination with a modified microorganism capable of producing one or more immune initiators. Alternatively, one or more first immune initiators may be administered in combination with a modified microorganism capable of producing one or more second immune initiators. Alternatively, one or more first immune sustainers may be administered in combination with a modified microorganism capable of producing one or more second immune sustainers.

Many immune cells found in the tumor microenvironment express pattern recognition receptors (PRRs), which receptors play a key role in the innate immune response through the activation of pro-inflammatory signaling pathways, stimulation of phagocytic responses (macrophages, neutrophils and dendritic cells) or binding to micro-organisms as secreted proteins. PRRs recognize two classes of molecules: (PAMPs), which are associated with microbial pathogens, and damage-associated molecular patterns (DAMPs), which are associated with cell components that are released during cell damage, death stress, or tissue injury. PAMPS are unique to each pathogen and are essential molecular structures required for the pathogens survival, e.g., bacterial cell wall molecules (e.g. lipoprotein), viral capsid proteins, and viral and bacterial DNA. PRRs can identify a variety of microbial pathogens, including bacteria, viruses, parasites, fungi, and protozoa. PRRs are primarily expressed by cells of the innate immune system, e.g., antigen presenting macrophage and dendritic cells, but can also be expressed by other cells (both immune and non-immune cells), and are either localized on the cell surface to detect extracellular pathogens or within the endosomes and cellular matrix where they detect intracellular invading viruses.

Examples of PRRs include Toll-like receptors (TLR), which are type 1 transmembrane receptors that have an extracellular domain which detects infecting pathogens. TLR1, 2, 4, and 6 recognize bacterial lipids, TLR3, 7 and 8 recognize viral RNA, TLR9 recognizes bacterial DNA, and TLR5 and 10 recognize bacterial or parasite proteins. Other examples of PRRs include C-type lectin receptors (CLR), e.g., group I mannose receptors and group II asialoglycoprotein receptors, cytoplasmic (intracellular) PRRs, nucleotide oligomerization (NOD)-like receptors (NLRs), e.g., NOD1 and NOD2, retinoic acid-inducible gene I (RIG-I)-like receptors (RLR), e.g., RIG-I, MDA5, and DDX3, and secreted PRRs, e.g., collectins, pentraxins, ficolins, lipid transferases, peptidoglycan recognition proteins (PGRs) and the leucine-rich repeat receptor (LRR).

PRRs initiate the activation of signaling pathways, such as the NF-kappa B pathway, that stimulates the production of co-stimulatory molecules and pro-inflammatory cytokines, e.g., type I IFNs, IL-6, TNF, and IL-12, which mechanisms play a role in the activation of inflammatory and immune responses mounted against infectious pathogens. Such response triggers the activation of immune cells present in the tumor microenvironment that are involved in the adaptive immune response (e.g., antigen-presenting cells (APCs) such as B cells, DCs, TAMs, and other myeloid derived suppressor cells). Recent evidence indicates that immune mechanisms activated by PAMPs and DAMPs play a role in activating immune responses against tumor cells as well (LeMercier et al., Canc Res, 73:4629-40 (2013); Kim et al., Blood, 119:355-63 (2012)).

Another PRR subfamily are the RIG-I-like receptors (RLRs) which are considered to be sensors of double-stranded viral RNA upon viral infection and which can be targeted for intratumoral immune stimulation. Upon stimulation, for example, upon intratumoral delivery of an oncolytic virus, RLRs trigger the release of type I IFNs by the host cell and result in its death by apoptosis. Such cytokine and tumor-associated antigen (TAA) release also results in the activation of the antitumor immune response. Given that RLRs are endogenously expressed in all tumor types, they are a universal proimmunogenic therapeutic target and of particular relevance in the immune response generated by local delivery of an oncolytic virus.

In some aspects, the bacterial chassis itself may activate one or more of the PRR receptors, e.g., TLRs or RIGI, and stimulate an innate immune response. In some aspects the PRRs, e.g., TLRs or RIGI, are activated by one or more immune modulators produced by the genetically engineered bacteria.

### Lytic Peptides

The bacteria of the present disclosure, by themselves, may result in cell lysis at the tumor site due to the presence of PAMPs and DAMPs, which will initiate an innate immune response. In addition, some bacteria have the added feature of being lytic microorganisms with the ability to lyse tumor cells. Thus, in some embodiments, the engineered microorganisms, produce natural or native lytic peptides. In some embodiments, the bacteria can be further engineered to produce one or more cytotoxic molecules, e.g., lytic peptides that have the ability to lyse cancer or tumor cells locally in the tumor microenvironment upon delivery to the tumor site. Upon cell lysis, the tumor cells release tumor-associated antigens that serve to promote an adaptive immune response. The presence of PAMPs and DAMPs promote the maturation of antigen-presenting cells, such as dendritic cells, which activate antigen-specific CD4+ and CD8+ T cell responses. Thus, in some embodiments, the genetically engineered bacteria are capable of producing one or more cytotoxin(s). In some embodiments, the genetically engineered bacteria or are capable of producing one or more lytic peptide molecule(s) Exemplary lytic peptide and cytotoxins which may be produced by the genetically engineered bacteria and how they may be expressed, induced and regulated, are described in International Patent Application PCT/US2017/013072, filed Jan. 11, 2017, published as WO2017/123675, and PCT/US2018/012698, filed Jan. 1, 2018, the contents of each of which is herein incorporated by reference in its entirety.

In any of these embodiments, the genetically engineered bacteria comprising gene sequence(s) encoding lytic peptides further comprise gene sequence(s) encoding one or more further effector molecule(s), i.e., therapeutic molecule(s) or a metabolic converter(s). In any of these embodiments, the circuit encoding lytic peptides may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding lytic peptides may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding lytic peptides encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding lytic peptides encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Antigens/Vaccines

By introducing tumor antigens, e.g., tumor-specific antigens, tumor-associated antigens (TAA(s)), and/or neoantigen(s) to the local tumor environment, an immune response can be raised against the particular cancer or tumor cell of interest known to be associated with that neoantigen. As used herein the term “tumor antigen” is meant to refer to tumor-specific antigens, tumor-associated antigens (TAAs), and neoantigens. As used herein, tumor antigen also includes “Oncogenic viral antigens”, Oncofetal antigens, tissue differentiation antigens, and cancer-testis antigens. The engineered microorganisms can be engineered such that the peptides, e.g. tumor antigens, can be anchored in the microbial cell wall (e.g., at the microbial cell surface). Thus, in some embodiments, the genetically engineered bacteria, are engineered to produce one or more tumor antigens. Non-limiting examples of such tumor antigens which may be produced by the bacteria of the disclosure described e.g., in International Patent Application PCT/US2017/013072, filed Jan. 11, 2017, published as WO2017/123675, and PCT/US2018/012698, filed Jan. 1, 2018, the contents of each of which is herein incorporated by reference in its entirety or otherwise known in the art.

In any of these embodiments, the genetically engineered bacteria comprising gene sequence(s) encoding antigens further comprise gene sequence(s) encoding one or more further effector molecule(s), i.e., therapeutic molecule(s) or a metabolic converter(s). In any of these embodiments, the circuit encoding antigens may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding antigens may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding antigens encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding antigens encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Prodrugs

Prodrug therapy provides less reactive and cytotoxic form of anticancer drugs. In some embodiments, the genetically engineered bacteria are capable of converting a prodrug into its active form. One example of a suitable prodrug system is the 5-FC/5-FU system.

The cytotoxic and radiosensitizing agent 5-fluorouracil (5-FU) is used in the treatment of many cancers including gastrointestinal, breast, head and neck and colorectal cancers (Duivenvorrden et al., 2006, Sensitivity of 5-fluorouracil-resistant cancer cells to adenovirus suicide gene therapy; Cancer Gene Therapy (2006) 14, 57-65). However, toxicity limits its administration at higher concentrations. In order to achieve higher concentrations at the tumor with less toxicity, a prodrug system was developed. Cytosine deaminase deaminates the prodrug 5-fluorocytosine (5-FC) into 5-FU. 5-FC can be introduced at relatively high concentrations, allowing the 5-FU generated at the tumor site to achieve concentrations that are higher than can be systemically administered safely. At the tumor site 5-FU is then transformed by cellular enzymes to potent pyrimidine antimetabolites, 5-FdUMP, 5-FdUTP and 5-FUTP. These metabolites act as metabolic blockers that inhibit thymidylate synthetase, which converts ribonucleotides to deoxyribonucleotides, thus inhibiting DNA synthesis ((Horani et al. 2015, Anticancer Prodrugs—Three Decades Of Design; wjpps; Volume 4, Issue 07, 1751-1779, and references therein).

This system has been further improved by the inclusion of the UPRT that converts 5-FU to 5-fluorouridine monophosphate, the first step of its pathway to activation, similar to the actions of the mammalian orotate phosphoribosyltransferase (Tiraby et al., 1998; Concomitant expression of E. coli cytosine deaminase and uracil phosphoribosyltransferase improves the cytotoxicity of 5-fluorocytosine. FEMS Microbiol Lett 1998; 176: 41-49).

In some embodiments, the genetically engineered bacteria are capable of converting 5-FC to 5FU. In some embodiments, the genetically engineered bacteria are capable of converting 5-FC to 5FU in the tumor microenvironment. In some embodiments, 5-FC is administered systemically. In some embodiments, 5-FC is administered orally, intravenously, or subcutaneously. In some embodiments, 5-FC is administered via intratumor injection. the genetically engineered bacteria comprise gene sequences encoding a cytosine deaminase (EC 3.5.4.1)

In some embodiments, the cytosine deaminase is from E. coli. In some embodiments, the cytosine deaminase is codA. In some embodiments, the genetically engineered bacteria express cytosine deaminase from yeast. In some embodiments, the genetically engineered bacteria express a codA-upp fusion protein.

Non-limiting examples of cytosine deaminases suitable for heterologous expression in the genetically engineered bacteria include Photobacterium leiognathi subsp. mandapamensis svers.1.1. (PMSV_1378), Pseudomonas mendocina NK-01 (MDS_1548), Streptomyces coelicolor A3(2) (SC04634), Achromobacter xylosoxidans AXX-A (AXXA_10715, AXXA_16292), Gluconacetobacter sp. SXCC-1 (CODA), Gallibacterium anatis UMN179 (UMN179_00049), Klebsiella oxytoca KCTC 1686 (KOX_14050, KOX_04555), Taylorella asinigenitalis MCE3 (TASI_1310), Rhodococcus jostii RHA1 (RHA1_R000599, RHA1_R000597), Enterobacter aerogenes KCTC 2190 (EAE_13265, EAE_05115), Candidatus Arthromitus sp. SFB-mouse-Japan (SFBM_1249), Ralstonia solanacearum Po82 (CODA), Salinisphaera shabanensis E1L3A (SSPSH_07086), Paenibacillus mucilaginosus KNP414 (KNP414_03230, KNP414_03233), Bradyrhizobium japonicum USDA 6 (BJ6T_60100, BJ6T_60090), Candidatus Arthromitus sp. SFB-rat-Yit (RATSFB_1079), Pseudomonas putida S16 (PPS_2740), Weissella koreensis KACC 15510 (WKK_05060), Enterobacter cloacae EcWSU1 (YAHJ, CODA), Bizionia argentinensis JUB59 (BZARG_2213), Agrobacterium tumefaciens F2 (AGAU_L101956), Paracoccus denitrificans SD1 (PDI_1216), Sulfobacillus acidophilus TPY (CODA), Vibrio tubiashii ATCC 19109 (VITU9109_13741), Nitrosococcus watsonii C-113 (NWAT_2475), Blattabacterium sp. (Mastotermes darwiniensis) str. MADAR (CODA), Blattabacterium sp. (Cryptocercus punctulatus) str. Cpu (CODA), Pelagibacterium halotolerans B2 (KKY_852, KKY_850), Burkholderia sp. YI23 (BYI23_A018410, BYI23_A008960), Synechococcus sp. CC9605 (SYNCC9605_0854), Pseudomonas fluorescens F113 (AEV61892.1), Vibrio sp. EJY3 (VEJY3_16491), Synechococcus elongatus PCC 7942 (SYNPCC7942_0568), Bradyrhizobium sp. ORS 278 (BRAD01789, BRADO0862), Synechocystis sp. PCC 6803 (CODA), Microcoleus chthonoplastes PCC 7420 (MC7420_274), Prochlorococcus marinus str. AS9601 (CODA), Escherichia coli O157:H7 str. EDL933 (YAHJ, CODA), Pseudomonas putida KT2440 (CODA), Synechococcus sp. WH 8109 (SH8109_1371), Prochlorococcus marinus subsp. marinus str. CCMP1375 (SSNA), Prochlorococcus marinus str. MIT 9515 (CODA), Prochlorococcus marinus str. MIT 9301 (CODA), Prochlorococcus marinus str. NATL1A (CODA), Agrobacterium tumefaciens str. C58 (ATU4698), Desulfobacterium autotrophicum HRM2 (CODA), Cyanobium sp. PCC 7001 (CPCC7001_2605), Yersinia pestis KIM10 (CODA), Clostridium perfringens ATCC 13124 (CODA), Nocardioides sp. JS614 (NOCA_1495), Corynebacterium efficiens YS-314 (CODA), Corynebacterium glutamicum ATCC 13032 (CGL0076, CODA), Bacillus anthracis str. Ames (BAS4389), Dickeya dadantii 3937 (CODA), Escherichia coli CFT073 (CODA, YAHJ), Trichodesmium erythraeum IMS101 (TERY_4570), Pseudomonas fluorescens Pf0-1 (CODA, PFL01_3146), Bifidobacterium longum NCC2705 (CODA), Carnobacterium sp. 17-4 (CAR_C04640, ATZC), Pseudomonas aeruginosa PAO1 (CODA), Clostridium tetani E88 (CTC_01883), Yersinia pestis C092 (CODA), Burkholderia cenocepacia J2315 (BCAM2780, CODA), Pseudomonas fluorescens SBW25 (CODA), Vibrio vulnificus CMCP6 (VV2_0789), Salmonella bongori NCTC 12419 (CODA), Salmonella enterica subsp. enterica serovar Typhi str. CT18 (CODA), Pseudomonas fluorescens Pf-5 (CODA), Oceanobacillus iheyensis HTE831 (OB1267), Synechococcus sp. R59916 (RS9916_32902), Synechococcus sp. RS9917 (RS9917_02061), Mannheimia succiniciproducens MBEL55E (SSNA), Vibrio parahaemolyticus RIMD 2210633 (VPA1243), Bradyrhizobium japonicum USDA 110 (BLL3846, BLL7276), Marinobacter adhaerens HP15 (HP15_2772), Enterococcus faecalis V583 3 seqs

EF_1061, EF_1062, EF_0390), Bacillus cereus ATCC 14579 (BC_4503), Synechococcus sp. CB0101 (SCB01_010100001875), Synechococcus sp. CB0205 (SCB02_010100013621), Burkholderia mallei ATCC 23344 (CODA), Labrenzia alexandrii DFL-11 (SADFL11_5050), Myxococcus xanthus DK 1622 (MXAN_5420), Ruegeria pomeroyi DSS-3 (SPO2806), Gloeobacter violaceus PCC 7421 (GLL2528), Streptomyces sp. C (SSNG_03287, SSNG_04186), Ralstonia eutropha JMP134 (REUT_B3993), Moorella thermoacetica ATCC 39073 (MOTH_0460), Rubrobacter xylanophilus DSM 9941 (RXYL_0224), Burkholderia xenovorans LB400 (BXE_A2120, BXE_A1533), Sinorhizobium meliloti 1021 (R02596), Mesorhizobium loti MAFF303099 (MLR5363, MLL2061), Ralstonia solanacearum GMI1000 (CODA), Synechococcus elongatus PCC 6301 (CODA), Burkholderia vietnamiensis G4 (BCEP1808_4874), Rhodospirillum rubrum ATCC 11170 (RRU_A2788), Marinobacter sp. ELB17 (MELB17_06099), Gluconacetobacter diazotrophicus PA1 5 (GDIA_2518, GD13632), Klebsiella pneumoniae subsp. pneumoniae MGH 78578 (KPN_00632, CODA), Pasteurella multocida subsp. multocida str. Pm70 (PM0565), Rhodobacter sphaeroides 2.4.1 (RSP_0341), Pediococcus pentosaceus ATCC 25745 (PEPE_0241), Pseudogulbenkiania ferrooxidans 2002 (FURADRAFT_0739), Desulfuromonas acetoxidans DSM 684 (DACE_0684), Aurantimonas manganoxydans SI85-9A1 (SI859A1_01947), Bradyrhizobium sp. BTAi1 (BBTA_2105, BBTA_7204), Cronobacter sakazakii ATCC BAA-894 (ESA_03405), Arthrobacter aurescens TC1 (AAUR_3889, AAUR_0925), Arthrobacter sp. FB24 (ARTH_3600), Jannaschia sp. CCS1 (JANN_1306), Polaromonas sp. JS666 (BPRO_1960), Photobacterium profundum SS9 (Y3946), Frankia sp. EuI1c (FRAEUI1C_4724, FRAEUI1C_4625), Thermomicrobium roseum DSM 5159 (TRD_1845), Agrobacterium vitis S4 (AVI_2101, AVI_2102), Agrobacterium radiobacter K84 5 seqs ARAD_9085, ARAD_9086, ARAD_8033, ARAD_3518, ARAD_9893), Vibrio fischeri ES114 (CODA), Lyngbya sp. PCC 8106 (L8106_10086), Synechococcus sp. BL107 (BL107_11056), Bacillus sp. NRRL B-14911 (B14911_04044), Roseobacter sp. MED193 (MED193_17224), Roseovarius sp. 217 (R05217_10957), Pelagibaca bermudensis HTCC2601 (R2601_16485, R2601_00530), Marinomonas sp. MED121 (MED121_23629), Lactobacillus sakei subsp. sakei 23K (LCA_1212), Bacillus weihenstephanensis KBAB4 (BCERKBAB4_4331), Rhodopseudomonas palustris HaA2 (RPB_2084), Aliivibrio salmonicida LFI1238 (CODA), Synechococcus sp. CC9902 (SYNCC9902_1538), Escherichia coli str. K-12 substr. W3110 (CODA, YAHJ), Paracoccus denitrificans PD1222 (PDEN_1057), Synechococcus sp. WH 7803 (CODA), Synechococcus sp. JA-3-3Ab (CYA_1567, CODA), Synechococcus sp. JA-2-3Ba(2-13) (CYB_1063, CODA), Brevibacterium linens BL2 (BLINB_010200009485), Azotobacter vinelandii DJ (CODA), Paenibacillus sp. JDR-2 6 seqs PJDR2_6131, PJDR2_6134, PJDR2_3617, PJDR2_3622, PJDR2_3255, PJDR2_3254), Frankia alni ACN14a (FRAAL4250), Bifidobacterium breve UCC2003 (CODA), Blattabacterium sp. (Blattella germanica) str. Bge (BLBBGE_353), alpha proteobacterium BAL199 (BAL199_01644, BAL199_09865), Carnobacterium sp. AT7 (CAT7_10495, CAT7_05806), Nitrosomonas eutropha C91 (NEUT_1722), Vibrio harveyi ATCC BAA-1116 (VIBHAR_05319), Burkholderia ambifaria AMMD (BAMB_3745, BAMB_4900), Actinobacillus succinogenes 130Z (ASUC_1190), Rhodobacter sphaeroides ATCC 17025 (RSPH17025_0955), Lactobacillus reuteri 100-23 (LR0661), Acidiphilium cryptum JF-5 (ACRY_0828), Hahella chejuensis KCTC 2396 (HCH_05147), Alkaliphilus oremlandii OhILAs (CLOS_1212, CLOS_2457), Burkholderia dolosa AU0158 (BDAG_04094, BDAG_03273), Roseobacter sp. AzwK-3b (RAZWK3B_08901), Pseudomonas putida Fl (PPUT_2527), Clostridium phytofermentans ISDg (CPHY_3622), Brevibacillus brevis NBRC 100599 4 seqs BBR47_15870, BBR47_15630, BBR47_15620, BBR47_15610), Bordetella avium 197N (CODA), Escherichia coli 536 (CODA, YAHJ), Polaromonas naphthalenivorans CJ2 (PNAP_4007), Ramlibacter tataouinensis TTB310 (CODA), Janthinobacterium sp. Marseille (CODA), Pseudomonas stutzeri A1501 (CODA), Aeromonas hydrophila subsp. hydrophila ATCC 7966 (CODA), Ralstonia eutropha H16 (CODA, SSNA), Pseudomonas entomophila L48 (PSEEN3598), Labrenzia aggregata IAM 12614 (SIAM614_16372, SIAM614_21000), Lactobacillus brevis ATCC 367 (LVIS_1932), Sagittula stellata E-37 (SSE37_18952), Bacillus sp. B14905 3 seqsBB14905_20948, BB14905_12010, BB14905_12015), Pseudomonas putida W619 3 seqs PPUTW619_3228, PPUTW619_2210, PPUTW619_2162), Stenotrophomonas maltophilia R551-3 (SMAL_2348), Burkholderia phymatum STM815 (BPHY_1477), Vibrionales bacterium SWAT-3 (VSWAT3_26556), Roseobacter sp. GAI101 (RGAI101_2568), Vibrio shilonii AK1 (VSAK1_17107), Pedobacter sp. BAL39 (PBAL39_00410), Roseovarius sp. TM1035 (RTM1035_18230, RTM1035_17900), Octadecabacter antarcticus 238 (OA238_4970), Phaeobacter gallaeciensis DSM 17395 (CODA), Oceanibulbus indolifex HEL-45 (OIHEL45_14065, OIHEL45_01925), Octadecabacter antarcticus 307 (OA307_78), Verminephrobacter eiseniae EF01-2 (VEIS_0416, VEIS_4430), Shewanella woodyi ATCC 51908 (SWOO_1853), Yersinia enterocolitica subsp. enterocolitica 8081 (CODA), Clostridium cellulolyticum H10 (CCEL_0909), Burkholderia multivorans ATCC 17616 (CODA, BMUL_4281), Leptothrix cholodnii SP-6 (LCHO_0318), Acidovorax citrulli AAC00-1 (AAVE_3221), Burkholderia phytofirmans PsJN (BPHYT_2598, BPHYT_2388), Delftia acidovorans SPH-1 (DACI_4995), Shewanella pealeana ATCC 700345 (SPEA_2187), Dinoroseobacter shibae DFL 12 (CODA), Pseudomonas mendocina ymp (PMEN_3834), Serratia proteamaculans 568 (SPRO_0096, SPRO_4594), Enterobacter sp. 638 (ENT638_3792, ENT638_3140), Marinomonas sp. MWYL1 (MMWYL1_1583), Saccharopolyspora erythraea NRRL 2338 (SERYN2_010100001217), Xenorhabdus nematophila ATCC 19061 (XNC1_2097), Nocardioidaceae bacterium Broad-1 (NBCG_02556), Hoeflea phototrophica DFL-43 (HPDFL43_16047), Paracoccus sp. TRP (PATRP_010100008956), Cyanothece sp. PCC 8801 (PCC8801_1952), Shewanella sediminis HAW-EB3 (SSED_2803), Methylobacterium sp. 4-46 (M446_3603, M446_0933), Methylobacterium radiotolerans JCM 2831 (MRAD2831_4824), Azorhizobium caulinodans ORS 571 (AZC_1945), Ochrobactrum anthropi ATCC 49188 (OANT_3311), Ruegeria sp. R11 (RR11_1621), Cyanothece sp. ATCC 51142 (CODA), Streptomyces clavuligerus ATCC 27064 (SCLAA2_010100026671, SCLAV_5539), Lysinibacillus sphaericus C3-41 (BSPH_4231), Clostridium botulinum NCTC 2916 (CODA), Anaerotruncus colihominis DSM 17241 (ANACOL_03998, ANACOL_02279, ANACOL_01309), Actinosynnema mirum DSM 43827 (AMIR_0538), Sanguibacter keddieii DSM 10542 (SKED_28020, SKED_17260), Stackebrandtia nassauensis DSM 44728 (SNAS_1703), Microcystis aeruginosa NIES-843 (MAE_05360), Clostridium perfringens NCTC 8239 (CODA), Kitasatospora setae KM-6054 (KSE_36300, KSE_36320), Arthrobacter chlorophenolicus A6 (ACHL_1061), Streptomyces griseus subsp. griseus NBRC 13350 (SGR_6458), Clostridium sp. 7_2_43 FAA (CSBG_02087), Clostridiales bacterium 1_7_47 FAA (CBFG_00901), Streptomyces albus J1074 (SSHG_05633), Shewanella halifaxensis HAW-EB4 (SHAL_2160), Methylobacterium nodulans ORS 2060 (MNOD_3349), Streptomyces sp. Mg1 (SSAG_05271), Erwinia tasmaniensis Et1/99 (CODA), Escherichia coli BL21(DE3) (YAHJ, CODA, B21_00295, B21_00283), Conexibacter woesei DSM 14684 (CWOE_5700, CWOE_5704, CWOE_0344), Citrobacter sp. 30_2 (CSAG_03013, CSAG_02691), Burkholderiales bacterium 1_1_47 (HMPREF0189_01313), Enterobacteriaceae bacterium 9_2_54 FAA (HMPREF0864_03568), Fusobacterium ulcerans ATCC 49185 (FUAG_02220), Fusobacterium varium ATCC 27725 (FVAG_00901), Beutenbergia cavernae DSM 12333 (BCAV_1683, BCAV_1451), Providencia stuartii ATCC 25827 (PROSTU_04183), Proteus penneri ATCC 35198 (PROPEN_03672), Streptosporangium roseum DSM 43021 (SROS_3184, SROS_4847), Paenibacillus sp. Y412MC10 (GYMC10_2692, GYMC10_4727, GYMC10_3398), Escherichia coli ATCC 8739 (YAHJ, CODA), Ktedonobacter racemifer DSM 44963 (KRAC_3038), Marinomonas posidonica IVIA-Po-181 (MAR181_2188), Cyanothece sp. PCC 7822 (CYAN7822_1898), Edwardsiella tarda EIB202 (CODA), Providencia rustigianii DSM 4541 (PROVRUST_05865), Enterobacter cancerogenus ATCC 35316 (ENTCAN_08376, ENTCAN_08631), Citrobacter youngae ATCC 29220 (CIT292_10672, CIT292_09697), Citreicella sp. SE45 (CSE45_2970), Escherichia albertii TW07627 (ESCAB7627_0317), Oligotropha carboxidovorans 0M5 (OCAR_4627, CODA), Escherichia coli str. K-12 substr. MG1655 (YAHJ, CODA), Lactobacillus buchneri NRRL B-30929 (LBUC_2038), Arthrospira maxima CS-328 (AMAXDRAFT_2897), Pantoea sp. aB (PANABDRAFT_0565, PANABDRAFT_2938), Eubacterium biforme DSM 3989 (EUBIFOR_01772), Providencia alcalifaciens DSM 30120 (PROVALCAL_01131, PROVALCAL_02804), Providencia rettgeri DSM 1131 (PROVRETT_08714, PROVRETT_08169), Stenotrophomonas maltophilia K279a (ATZC2), Anaerococcus lactolyticus ATCC 51172 (CODA), Anaerococcus tetradius ATCC 35098 (HMPREF0077_0097), Chryseobacterium gleum ATCC 35910 (DAN2), Lactobacillus buchneri ATCC 11577 (CODA), Lactobacillus vaginalis ATCC 49540 (CODA), Listeria grayi DSM 20601 (HMPREF0556_10753, HMPREF0556_10751, ATZC), Desulfomicrobium baculatum DSM 4028 (DBAC_2936), Anaerococcus prevotii DSM 20548 (APRE_1112), Sebaldella termitidis ATCC 33386 (STERM_0789), Meiothermus silvanus DSM 9946 (MESIL_2103), Proteus mirabilis HI4320 (CODA), Mesorhizobium opportunistum WSM2075 (MESOP_0162), Variovorax paradoxus 5110 (VAPAR_2654), Bacillus megaterium QM B1551 (BMQ_0980), Bifidobacterium pseudocatenulatum DSM 20438=JCM 1200 (BIFPSEUDO_04382), Ferrimonas balearica DSM 9799 (FBAL_2173), Ruminococcaceae bacterium D16 (HMPREF0866_00501), Photorhabdus asymbiotica subsp. asymbiotica ATCC 43949 (PAU_00294), Halothiobacillus neapolitanus c2 (HNEAP_0844), Haemophilus parasuis SH0165 (CODA), Dickeya zeae Ech1591 (DD1591_0763), Bilophila wadsworthia 3_1_6 (HMPREF0179_03393), Enterococcus gallinarum EG2 (EGBG_00349), Enterococcus casseliflavus EC20 (ECBG_00307), Spirochaeta smaragdinae DSM 11293 (SPIRS_1052, SPIRS_0110), Acinetobacter junii SH205 (HMPREF0026_02783), Vibrio splendidus LGP32 (VS_II0327), Dickeya dadantii Ech703 (DD703_0777), Moritella sp. PE36 (PE36_15643), Hirschia baltica ATCC 49814 (HBAL_0036), Aminomonas paucivorans DSM 12260 (APAU_2064), Weissella paramesenteroides ATCC 33313 (CODA), Dickeya dadantii Ech586 (DD586_3388), Streptomyces sp. SPB78 (SSLG_06016), Streptomyces sp. AA4 (SSMG_05855, SSMG_03227), Streptomyces viridochromogenes DSM 40736 (SSQG_04727), Streptomyces flavogriseus ATCC 33331 (SFLA_1190), Anaerobaculum hydrogeniformans ATCC BAA-1850 (HMPREF1705_02256), Pantoea sp. At-9b (PAT9B_3678, PAT9B_1029, PAT9B_0855), Variovorax paradoxus EPS (VARPA_3257, VARPA_0920), Prochlorococcus marinus subsp. pastoris str. CCMP1986 (CODA), Synechococcus sp. WH 7805 (WH7805_05676), Blattabacterium sp. (Periplaneta americana) str. BPLAN (CODA), Burkholderia glumae BGR1 (BGLU_1 G17900), Azoarcus sp. BH72 (CODA), Clostridium butyricum E4 str. BoNT E BL5262 (CODA), Erwinia pyrifoliae Ep1/96 (CODA), Erwinia billingiae Eb661 (EBC_35430, CODA, EBC_32850, EBC_32780), Edwardsiella ictaluri 93-146 (NT01EI_3615), Citrobacter rodentium ICC168 (CODA), Starkeya novella DSM 506 (SNOV_3614, SNOV_2304), Burkholderia sp. CCGE1001 (BC1001_2311), Burkholderia sp. CCGE1002 (BC1002_1908, BC1002_1610), Burkholderia sp. CCGE1003 (BC1003_1147), Enterobacter asburiae LF7a (ENTAS_4074, ENTAS_3370), Ochrobactrum intermedium LMG 3301 (OINT_2000395, OINT_2001541), Clostridium lentocellum DSM 5427 (CLOLE_1291), Desulfovibrio aespoeensis Aspo-2 (DAES_2101), Gordonia neofelifaecis NRRL B-59395 (SCNU_19677), Synechococcus sp. CC9311 (SYNC_0740), Thermaerobacter marianensis DSM 12885 (TMAR_1477), Rhodomicrobium vannielii ATCC 17100 (RVAN_3395), Bacillus cellulosilyticus DSM 2522 (BCELL_1091, BCELL_1234), Cyanothece sp. PCC 7424 (PCC7424_0235), Lachnospiraceae bacterium 3_1_57 FAA_CT1 (HMPREF0994_04419), Bacillus sp. 2_A_57_CT2 (HMPREF1013_04901, HMPREF1013_04902, HMPREF1013_01532, HMPREF1013_04888), Afipia sp. 1NLS2 (AFIDRAFT_3092), Bacillus clausii KSM-K16 (ABC4032), Serratia odorifera DSM 4582 (YAHJ, CODA), Vibrio alginolyticus 40B (VMC_19080), Pseudonocardia dioxanivorans CB1190 (PSED_5383), Vibrio coralliilyticus ATCC BAA-450 (VIC_002709), Vibrio orientalis CIP 102891=ATCC 33934 (VIA_000851), Photobacterium damselae subsp. damselae CIP 102761 (VDA_000799), Prevotella buccalis ATCC 35310 (HMPREF0650_2329), Serratia odorifera 4Rx13 (SOD_G01050, SOD_H00810), Synechococcus sp. WH 5701 (WH5701_16173, WH5701_07386), Arthrospira platensis NIES-39 (BAI89358.1), Vibrio sp. N418 (VIBRN418_08807), Enterobacter cloacae SCF1 (ENTCL_0362), Pediococcus claussenii ATCC BAA-344 (CODA), Pantoea ananatis LMG 20103 (CODA, YAHJ), Bradyrhizobiaceae bacterium SG-6C (CSIRO_2009), Pantoea vagans C9-1 (CODA, YAHJ), Lactobacillus fermentum CECT 5716 (LC40_0597), Lactobacillus iners AB-1 (LINEA_010100006044), Lysinibacillus fusiformis ZC1 (BFZC1_05123, BFZC1_05118), Paenibacillus vortex V453 (PVOR_16204, PVOR_25863), Enterobacter cloacae subsp. cloacae ATCC 13047 (ECL_04741, ECL_03997), Marinomonas mediterranea MMB-1 (MARME_0493), Enterobacter cloacae subsp. cloacae NCTC 9394 (ENC_29090, ENC_34640), Rahnella sp. Y9602 (RAHAQ_4063, RAHAQ_0278), Achromobacter piechaudii ATCC 43553 (HMPREF0004_2397, ATZC, CODA), Sutterella wadsworthensis 3_1_45 B (HMPREF9464_00595), Pseudomonas fulva 12-X (PSEFU_1564), Rahnella aquatilis CIP 78.65=ATCC 33071 (AEX50243.1, AEX53933.1), Prochlorococcus marinus str. MIT 9312 (PMT9312_1400), Prochlorococcus marinus str. MIT 9313 (CODA), Pseudomonas fluorescens WH6 (YAHJ), Clostridium ljungdahlii DSM 13528 (CLJU_C19230), Streptomyces bingchenggensis BCW-1 (SBI_06150), Amycolatopsis mediterranei U32 (AMED_1997), Microcoleus vaginatus FGP-2 (MICVADRAFT_2986, MICVADRAFT_1253), Ketogulonigenium vulgarum WSH-001 (CODAB, KVU_1143), Achromobacter xylosoxidans A8 (AXYL_01223, AXYL_05738, AXYL_01981, CODA), Pedobacter saltans DSM 12145 (PEDSA_0106), Mesorhizobium ciceri biovar biserrulae WSM1271 (MESCI_0163), Pseudomonas putida GB-1 (PPUTGB1_2651, PPUTGB1_3590), Xanthobacter autotrophicus Py2 (XAUT_4058), Synechococcus sp. WH 8102 (CODA), Corynebacterium variabile DSM 44702 (CODA), Agrobacterium sp. H13-3 (AGROH133_09551), Pediococcus acidilactici DSM 20284 (CODA), Haemophilus parainfluenzae T3T1 (PARA_18250), Weeksella virosa DSM 16922 (WEEVI_1993), Aerococcus urinae ACS-120-V-Col10a (CODA), Thermaerobacter subterraneus DSM 13965 (THESUDRAFT_1163), Aeromonas caviae Ae398 (ACAVA_010100000636), Burkholderia rhizoxinica HKI 454 (RBRH_03808), Salmonella enterica subsp. arizonae serovar str. RSK2980 (SARI_04290), Hylemonella gracilis ATCC 19624 (HGR_11321), Aggregatibacter segnis ATCC 33393 (CODA), Roseovarius nubinhibens ISM (ISM_11230), Plautia stali symbiont (PSTAS_010100016161, PSTAS_010100013574), Peptoniphilus harei ACS-146-V-Sch2b (CODA), Pseudovibrio sp. FO-BEG1 (PSE_0768), Weissella cibaria KACC 11862 (WCIBK1_010100001529), Synechococcus sp. PCC 7335 (S7335_2052, S7335_109, S7335_1731), Anaerolinea thermophila UNI-1 (ANT_02950), Prochlorococcus marinus str. MIT 9211 (CODA), Prochlorococcus marinus str. MIT 9215 (CODA), Fructobacillus fructosus KCTC 3544 (FFRUK3_010100004834), Lactobacillus farciminis KCTC 3681 (LFARK3_010100001847), Lactobacillus fructivorans KCTC 3543 (LFRUK3_010100002075), Tetragenococcus halophilus NBRC 12172 (TEH_05430, TEH_14850, TEH_02220), Vibrio brasiliensis LMG 20546 (VIBRO546_14545), Cupriavidus taiwanensis LMG 19424 (CODA), Microbacterium testaceum StLB037 (MTES_1247, MTES_3600), Paenibacillus terrae HPL-003 (HPL003_22070), Rubrivivax benzoatilyticus JA2 (RBXJA2T_04743), Polymorphum gilvum SL003B-26A1 (SL003B_2461), Salmonella enterica subsp. enterica serovar Typhimurium str. LT2 (STM3334), Streptomyces griseoaurantiacus M045 (SGM_3210), Aeromonas veronii B565 (B565_3987), Halomonas sp. TD01 (GME_08209), Burkholderia gladioli BSR3 (BGLA_2 G13660).

In some embodiments, the genetically engineered bacteria are administered intratumorally and 5-FC is administered systemically. In some embodiments, both the genetically engineered bacteria and 5-FC are administered systemically.

In any of these embodiments, the bacteria genetically engineered to produce 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more 5-FU from 5-FC than unmodified bacteria of the same bacterial subtype under the same conditions, e.g., under in vitro or in vivo conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more 5-FU from 5-FC than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more 5-FU from 5-FC than unmodified bacteria of the same bacterial subtype under the same conditions, e.g. under in vitro or in vivo conditions.

In any of these embodiments, the bacteria genetically engineered to produce 5-FU consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% or more increased amounts of 5-FC than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more 5-FC than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold or more increased amounts of 5-FC than unmodified bacteria of the same bacterial subtype under the same conditions.

In any of these embodiments, the genetically engineered bacteria comprising gene sequences encoding a circuit for the conversion of 5-FC to 5-FU are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria comprising gene sequences encoding a circuit for the conversion of 5-FC to 5-FU are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria comprising gene sequences encoding a circuit for the conversion of 5-FC to 5-FU are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these conversion embodiments, the genetically engineered bacteria comprising gene sequences encoding a circuit for the conversion of 5-FC to 5-FU are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria comprising gene sequences encoding a circuit for the conversion of 5-FC to 5-FU are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria comprise a gene sequence encoding CodA. In one embodiment, the CodA gene has at least about 80% identity with a SEQ ID NO: 1213. In another embodiment, the CodA gene has at least about 85% identity with SEQ ID NO: 1213. In one embodiment, the CodA gene has at least about 90% identity with SEQ ID NO: 1213. In one embodiment, the CodA gene has at least about 95% identity with SEQ ID NO: 1213. In another embodiment, the CodA gene has at least about 96%, 97%, 98%, or 99% identity with SEQ ID NO: 1213. Accordingly, in one embodiment, the CodA gene has at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1213. In another embodiment, the CodA gene comprises the sequence of SEQ ID NO: 1213. In yet another embodiment, the CodA gene consists of the sequence of SEQ ID NO: 1213.

In some embodiments, the genetically engineered bacteria comprise a gene sequence encoding a CodA polypeptide having at least about 80% identity with SEQ ID NO: 1216 OR SEQ ID NO: 1217. In some embodiments, the genetically engineered bacteria comprise a gene sequence encoding a CodA polypeptide that has about having at least about 90% identity with SEQ ID NO: 1216 OR SEQ ID NO: 1217. In some embodiments, the genetically engineered bacteria comprise a gene sequence encoding a CodA polypeptide that has about having at least about 95% identity with SEQ ID NO: 1216 OR SEQ ID NO: 1217. In some embodiments, the genetically engineered bacteria comprise a gene sequence encoding a CodA polypeptide that has about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1216 OR SEQ ID NO: 1217, or a functional fragment thereof. In another embodiment, the genetically engineered bacteria comprise a gene sequence encoding a CodA polypeptide comprising SEQ ID NO: 1216 OR SEQ ID NO: 1217. In yet another embodiment, the polypeptide expressed by the genetically engineered bacteria consists of SEQ ID NO: 1216 OR SEQ ID NO: 1217.

In some embodiments, cytosine deaminases are modified and/or mutated, e.g., to enhance stability, or to increase 5-FU production. In some embodiments, the genetically engineered bacteria and/or other microorganisms are capable of producing the cytosine deaminases under inducing conditions, e.g., under a condition(s) associated with immune suppression and/or tumor microenvironment. In some embodiments, the genetically engineered bacteria and/or other microorganisms are capable of producing the cytosine deaminases in low-oxygen conditions or hypoxic conditions, in the presence of certain molecules or metabolites, in the presence of molecules or metabolites associated with cancer, or certain tissues, immune suppression, or inflammation, or in the presence of some other metabolite that may or may not be present in the gut, circulation, or the tumor, such as arabinose, cumate, and salicylate.

In some embodiments, the genetically engineered bacteria encode cytosine deaminases from E. coli. In some embodiments, cytosine deaminase from E. coli is modified and/or mutated, e.g., to enhance stability, or to increase 5-FU production. In some embodiments, the genetically engineered bacteria and/or other microorganisms are capable of producing the cytosine deaminases under inducing conditions, e.g., under a condition(s) associated with immune suppression and/or tumor microenvironment. In some embodiments, the genetically engineered bacteria and/or other microorganisms are capable of producing cytosine deaminase, in low-oxygen conditions or hypoxic conditions, in the presence of certain molecules or metabolites, in the presence of molecules or metabolites associated with cancer, or certain tissues, immune suppression, or inflammation, or in the presence of some other metabolite that may or may not be present in the gut, circulation, or the tumor, such as arabinose, cumate, and salicylate.

In some embodiments, the genetically engineered bacteria and/or other microorganisms are capable of expressing any one or more of the described circuits, including but not limited to, circuitry for the expression of cytosine deaminases, from E. coli, in low-oxygen conditions, and/or in the presence of cancer and/or the tumor microenvironment and/or the tumor microenvironment or tissue specific molecules or metabolites, and/or in the presence of molecules or metabolites associated with inflammation or immune suppression, and/or in the presence of metabolites that may be present in the gut or the tumor, and/or in the presence of metabolites that may or may not be present in vivo, and may be present in vitro during strain culture, expansion, production and/or manufacture, such as arabinose, cumate, and salicylate and others described herein. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during bacteria and/or other microorganisms expansion, production and/or manufacture, as described herein. In any of these embodiments, any one or more of the described circuits, including but not limited to, circuitry for the expression of cytosine deaminases, e.g., from E. coli, are present on one or more plasmids (e.g., high copy or low copy) or are integrated into one or more sites in the bacteria and/or other microorganism chromosome(s).

In any of these embodiments, the genetically engineered bacteria comprising gene sequence(s) encoding cytosine deaminases further comprise gene sequence(s) encoding one or more further effector molecule(s), i.e., therapeutic molecule(s) or a metabolic converter(s). In any of these embodiments, the circuit encoding cytosine deaminases may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding cytosine deaminases may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding cytosine deaminases encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding cytosine deaminases encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In some embodiments, the genetically engineered bacteria and/or other microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art (7) one or more circuits for the production or degradation of one or more metabolites (e.g., kynurenine, tryptophan, adenosine, arginine) described herein and (8) combinations of one or more of such additional circuits. In any of these embodiments, the genetically engineered bacteria may be administered alone or in combination with one or more immune checkpoint inhibitors described herein, including but not limited to anti-CTLA4 antibodies or anti-PD1 or anti-PDL1 antibodies.

### Inhibition of Phagocytosis Escape—CD47-SIRPα Pathway

Cancers have the ability to up-regulate the “don't eat me” signal to allow escape from endogenous “eat me” signals that were induced as part of programmed cell death and programmed cell removal, to promote tumor progression.

CD47 is a cell surface molecule implicated in cell migration and T cell and dendritic cell activation. In addition, CD47 functions as an inhibitor of phagocytosis through ligation of signal-regulatory protein alpha (SIRPα) expressed on phagocytes, leading to tyrosine phosphatase activation and inhibition of myosin accumulation at the submembrane assembly site of the phagocytic synapse. As a result, CD47 conveys a “don't eat me signal”. Loss of CD47 leads to homeostatic phagocytosis of aged or damaged cells.

Elevated levels of CD47 expression are observed on multiple human tumor types, allowing tumors to escape the innate immune system through evasion of phagocytosis. This process occurs through binding of CD47 on tumor cells to SIRPα on phagocytes, thus promoting inhibition of phagocytosis and tumor survival.

Anti-CD47 antibodies have demonstrated pre-clinical activity against many different human cancers both in vitro and in mouse xenotransplantation models (Chao et al., Curr Opin Immunol. 2012 April; 24(2): 225-232. The CD47-SIRPα Pathway in Cancer Immune Evasion and Potential Therapeutic Implications, and references therein). In addition to CD47, SIRPα can also be targeted as a therapeutic strategy; for example, anti-SIRPα antibodies administered in vitro caused phagocytosis of tumor cells by macrophages (Chao et al., 2012).

In a third approach, CD47-targeted therapies have been developed using the single 14 kDa CD47 binding domain of human SIRPα (a soluble form without the transmembrane portion) as a competitive antagonist to human CD47 (as described in Weiskopf et al., Engineered SIRPα variants as immunotherapeutic adjuvants to anti-cancer antibodies; Science. 2013 Jul. 5; 341(6141): 10.1126/science.1238856, the contents of which is herein incorporated by reference in its entirety). Because the wild type SIRPα showed relatively low affinity to CD47, mutated SIRPα were generated through in vitro evolution via yeast surface display, which were shown to act as strong binders and antagonists of CD47. These variant include CV1 (consensus variant 1) and high-affinity variant FD6, and Fc fusion proteins of these variants. The amino acid changes leading to the increased affinity are located in the dl domain of human SIRPα. Non-limiting examples of SIRPα variants are also described in WO/2013/109752, the contents of which is herein incorporated by reference in its entirety.

In certain embodiments, the genetically engineered bacteria produce one or more immune modulators that inhibit CD47 and/or inhibit SIRPα and/or inhibit or prevent the interaction between CD47 and SIRPα expressed on macrophages. For example, the genetically engineered microorganism may encode an antibody directed against CD47 and/or an antibody directed against SIRPα, e.g. a single-chain antibody against CD47 and/or a single-chain antibody against SIRPα. In another non-limiting example, the genetically engineered microorganism may encode a competitive antagonist polypeptide comprising the SIRPα CD47 binding domain. Such a competitive antagonist polypeptide can function through competitive binding of CD47, preventing the interaction of CD47 with SIRPα expressed on macrophages. In some embodiments, the competitive antagonist polypeptide is soluble, e.g., is secreted from the microorganism. In some embodiments, the competitive antagonist polypeptide is displayed on the surface of the microorganism. In some embodiments, the genetically engineered microorganism encoding the competitive antagonist polypeptide encodes a wild type form of the SIRPα CD47 binding domain. In some embodiments, the genetically engineered microorganism encoding the competitive antagonist polypeptide encodes a mutated or variant form of the SIRPα CD47 binding domain. In some embodiments, the variant form is the CV1 SIRPα variant. In some embodiments, the variant form is the FD6 variant. In some embodiments, the SIRPα variant is a variant described in Weiskopf et al., and/or International Patent Publication WO/2013/109752. In some embodiments, the genetically engineered microorganism encoding the competitive antagonist polypeptide encodes a SIRPα CD47 binding domain or variant thereof fused to a stabilizing polypeptide. In some embodiments, the genetically engineered microorganism encoding the competitive antagonist polypeptide encodes a wild type form of the SIRPα CD47 binding domain fused to a stabilizing polypeptide. In a non-limiting example, the stabilizing polypeptide fused to the wild type SIRPα CD47 binding domain polypeptide is a Fc portion. In some embodiments, the stabilizing polypeptide fused to the wild type SIRPα CD47 binding domain polypeptide is the IgG Fc portion. In some embodiments, the stabilizing polypeptide fused to the wild type SIRPα CD47 binding domain polypeptide is the IgG4 Fc portion. In some embodiments, the genetically engineered microorganism encoding the competitive antagonist polypeptide encodes a mutated or variant form of the SIRPα CD47 binding domain fused to a stabilizing polypeptide. In some embodiments, the variant form fused to the stabilizing polypeptide is the CV1 SIRPα variant. In some embodiments, the variant form fused to the stabilizing polypeptide is the F6 variant. In some embodiments, the SIRPα variant fused to the stabilizing polypeptide is a variant described in Weiskopf et al., and/or International Patent Publication WO/2013/109752. In a non-limiting example, the stabilizing polypeptide fused to the variant SIRPα CD47 binding domain polypeptide is a Fe portion. In some embodiments, the stabilizing polypeptide fused to the variant SIRPα CD47 binding domain polypeptide is the IgG Fc portion. In some embodiments, the stabilizing polypeptide fused to the variant SIRPα CD47 binding domain polypeptide is an IgG4 Fc portion.

In some embodiments, the genetically engineered bacterium is bacterium that expresses an anti-CD47 antibody and/or anti-SIRPα antibody, e.g., a single chain antibody. In some embodiments, the genetically engineered bacterium is bacterium that expresses competitive antagonist SIRPα CD47 binding domain (WT or mutated to improve CD47 affinity). In some embodiments, the genetically engineered bacterium is bacterium that expresses an anti-CD47 antibody and/or anti-SIRPα antibody, e.g., a single chain antibody, under the control of a promoter that is activated by low-oxygen conditions. In some embodiments, the genetically engineered bacterium expresses a competitive antagonist SIRPα CD47 binding domain (WT or mutated variant with improved CD47 affinity) under the control of a promoter that is activated by low-oxygen conditions. In some embodiments, the genetically engineered bacterium expresses an anti-CD47 antibody and/or an anti-SIRPα, e.g., single chain antibody, under the control of a promoter that is activated by hypoxic conditions, or by inflammatory conditions, such as any of the promoters activated by said conditions and described herein. In some embodiments, the genetically engineered bacterium expresses a competitive antagonist SIRPα CD47 binding domain (WT or mutated variant with improved CD47 affinity) under the control of a promoter that is activated by hypoxic conditions, or by inflammatory conditions, such as any of the promoters activated by said conditions and described herein. In some embodiments, the genetically engineered bacteria expresses an anti-CD47antibody and/or an anti-SIRPα antibody, e.g., single chain antibody, under the control of a cancer-specific promoter, a tissue-specific promoter, or a constitutive promoter, such as any of the promoters described herein. In some embodiments, the genetically engineered bacteria comprise one or more genes encoding a competitive antagonist SIRPα CD47 binding domain (WT or mutated variant with improved CD47 affinity) under the control of a cancer-specific promoter, a tissue-specific promoter, or a constitutive promoter, such as any of the promoters described herein. In any of these embodiments, the genetically engineered microorganisms may also produce one or more immune modulators that are capable of stimulating Fc-mediated functions such as ADCC, and/or M-CSF and/or GM-CSF, resulting in a blockade of phagocytosis inhibition.

The genetically engineered bacteria and/or other microorganisms may comprise one or more genes encoding any suitable anti-CD47 antibody, anti-SIRPα antibody or competitive SIRPα CD47 binding domain polypeptide (wild type or mutated variant with improved CD47 binding affinity) for the inhibition or prevention of the CD47-SIRPα interaction. In some embodiments, the antibody(ies) or competitive polypeptide(s) is modified and/or mutated, e.g., to enhance stability, increase CD47 antagonism. In some embodiments, the genetically engineered bacteria and/or other microorganisms are capable of producing the antibody(ies) or competitive polypeptide(s) under inducing conditions, e.g., under a condition(s) associated with immune suppression and/or tumor microenvironment. In some embodiments, the genetically engineered bacteria and/or other microorganisms are capable of producing the antibody(ies) or competitive polypeptide(s) in low-oxygen conditions or hypoxic conditions, in the presence of certain molecules or metabolites, in the presence of molecules or metabolites associated with cancer, or certain tissues, immune suppression, or inflammation, or in the presence of some other metabolite that may or may not be present in the gut, circulation, or the tumor, such as arabinose, cumate, and salicylate.

In some embodiments, the genetically engineered bacteria comprise an anti-CD47 gene sequence encoding B6H12-anti-CD47-scFv. In some embodiments, the genetically engineered bacteria encode a polypeptide which is at least about 80%, at least about 85%, at least about 90%, at least about 95%, or at least about 99% homologous to SEQ ID NO: 994. In some embodiments, the genetically engineered bacteria encode a polypeptide comprising SEQ ID NO: 994. In some embodiments, the genetically engineered bacteria encode a polypeptide consisting of SEQ ID NO: 994. In some embodiments, the genetically engineered bacteria comprise an anti-CD47 gene sequence encoding 5F9-anti-CD47-scFv. In some embodiments, the genetically engineered bacteria encode a polypeptide which is at least about 80%, at least about 85%, at least about 90%, at least about 95%, or at least about 99% homologous to a sequence selected from SEQ ID NO: 996. In some embodiments, the genetically engineered bacteria encode a polypeptide comprising SEQ ID NO: 996. In some embodiments, the genetically engineered bacteria encode a polypeptide consisting of SEQ ID NO: 996. In some embodiments, the genetically engineered bacteria comprise an anti-CD47 gene sequence encoding 5F9antihCD47scFv-V5-HIS. In some embodiments, the Anti-CD47 scFv sequences is at least about 80%, at least about 85%, at least about 90%, at least about 95%, or at least about 99% homologous to a sequence selected from SEQ ID NO: 993 and SEQ ID NO: 995, excluding the non-coding regions and sequences coding for tags. In some embodiments, the gene sequence comprises a sequence selected from SEQ ID NO: 993 and SEQ ID NO: 995, excluding the non-coding regions and sequences coding for tags. In some embodiments, the gene sequence consists of a sequence selected from SEQ ID NO: 993 and SEQ ID NO: 995, excluding the non-coding regions and sequences coding for tags.

In some embodiments, the genetically engineered bacteria comprise a gene sequence encoding a SIRPα polypeptide having at least about 80% identity with a sequence selected from SEQ ID NO: 1118, SEQ ID NO: 1231, SEQ ID NO: 1119, SEQ ID NO: 1120. In some embodiments, the genetically engineered bacteria comprise a gene sequence encoding a SIRPα polypeptide having at least about 90% identity with a sequence selected from SEQ ID NO: 1118, SEQ ID NO: 1231, SEQ ID NO: 1119, SEQ ID NO: 1120. In some embodiments, the genetically engineered bacteria comprise a gene sequence encoding a SIRPα polypeptide having at least about 95% identity with a sequence selected from SEQ ID NO: 1118, SEQ ID NO: 1231, SEQ ID NO: 1119, SEQ ID NO: 1120. In some embodiments, the genetically engineered bacteria comprise a gene sequence encoding a SIRPα polypeptide that has about 80%, 81%, 82%, 83%, 84%, 85%, 86%, 87%, 88%, 89%, 90%, 91%, 92%, 93%, 94%, 95%, 96%, 97%, 98%, or 99% identity a to a sequence selected from SEQ ID NO: 1118, SEQ ID NO: 1231, SEQ ID NO: 1119, SEQ ID NO: 1120, or a functional fragment thereof. In another embodiment, the SIRPα polypeptide comprises a sequence selected from SEQ ID NO: 1118, SEQ ID NO: 1231, SEQ ID NO: 1119, and SEQ ID NO: 1120. In yet another embodiment, the polypeptide expressed by the genetically engineered bacteria consists of a sequence selected from SEQ ID NO: 1118, SEQ ID NO: 1231, SEQ ID NO: 1119, and SEQ ID NO: 1120.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions.

In any of these embodiments, the bacteria genetically engineered to produce SIRPα, SIRPα variant (e.g., CV1 or FD6 variant), or SIRPα-fusion protein (e.g., SIRPα IgG Fc fusion protein) secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions. In yet another embodiment, the genetically engineered bacteria secrete three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more SIRPα, SIRPα variant (e.g., CV1 or FD6 variant), or SIRPα-fusion protein (e.g., SIRPα IgG Fc fusion protein) than unmodified bacteria of the same bacterial subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce secrete SIRPα, SIRPα variant (e.g., CV1 or FD6 variant), or SIRPα-fusion protein (e.g., SIRPα IgG Fc fusion protein) are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete SIRPα, SIRPα variant (e.g., CV1 or FD6 variant), or SIRPα-fusion protein (e.g., SIRPα IgG Fc fusion protein) are capable of increasing phagocytosis of tumor cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more anti-CD47 scFv than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more anti-CD47 scFv than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more anti-CD47 scFv than unmodified bacteria of the same bacterial subtype under the same conditions.

In any of these embodiments, the bacteria genetically engineered to produce anti-CD47 scFv secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more anti-CD47 scFv than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more anti-CD47 scFv than unmodified bacteria of the same bacterial subtype under the same conditions. In yet another embodiment, the genetically engineered bacteria secrete three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more anti-CD47 scFv than unmodified bacteria of the same bacterial subtype under the same conditions.

In some embodiments, the bacteria genetically engineered to secrete anti-CD47 scFv are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete anti-CD47 scFv are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete anti-CD47 scFv are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete anti-CD47 scFv are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete anti-CD47 scFv are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce anti-CD47 scFv are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete anti-CD47 scFv are capable of increasing phagocytosis of tumor cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In yet another embodiment, the genetically engineered bacteria increase phagocytosis of tumor cells by at least 1.0-1.2-fold, 1.2-1.4-fold, 1.4-1.6-fold, 1.6-1.8-fold, 1.8-2-fold, or two-fold more than unmodified bacteria of the same bacterial subtype under the same conditions. In yet another embodiment, the genetically engineered bacteria increase phagocytosis of tumor cells three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more than unmodified bacteria of the same bacterial subtype under the same conditions.

In some embodiments, the genetically engineered bacteria and/or other microorganisms are capable of expressing any one or more of the described SIRPα or anti-CD47 circuits in low-oxygen conditions, and/or in the presence of cancer and/or the tumor microenvironment and/or the tumor microenvironment or tissue specific molecules or metabolites, and/or in the presence of molecules or metabolites associated with inflammation or immune suppression, and/or in the presence of metabolites that may be present in the gut or the tumor, and/or in the presence of metabolites that may or may not be present in vivo, and may be present in vitro during strain culture, expansion, production and/or manufacture, such as arabinose, cumate, and salicylate and others described herein. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during bacteria and/or other microorganismal expansion, production and/or manufacture, as described herein. In some embodiments, the gene sequences are present on one or more plasmids (e.g., high copy or low copy) or are integrated into one or more sites in the bacteria and/or other microorganism chromosome(s).

In any of these embodiments, the genetically engineered bacteria comprising gene sequence(s) encoding SIRPα or variants thereof or anti-CD47 polypeptides further comprise gene sequence(s) encoding one or more further effector molecule(s), i.e., therapeutic molecule(s) or a metabolic converter(s). In any of these embodiments, the circuit encoding SIRPα or variants thereof or anti-CD47 polypeptides may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding SIRPα or variants thereof or anti-CD47 polypeptides may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding SIRPα or variants thereof or anti-CD47 polypeptides encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding SIRPα or variants thereof or anti-CD47 polypeptides encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In some embodiments, any one or more of the described circuits are present on one or more plasmids (e.g., high copy or low copy) or are integrated into one or more sites in the bacteria and/or other microorganism chromosome(s). Also, in some embodiments, the genetically engineered bacteria and/or other microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art (7) one or more circuits for the production or degradation of one or more metabolites (e.g., kynurenine, tryptophan, adenosine, arginine) described herein and (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Activation of Antigen Presenting Cells

### STING Agonists

Stimulator of interferon genes (STING) protein was shown to be a critical mediator of the signaling triggered by cytosolic nucleic acid derived from DNA viruses, bacteria, and tumor-derived DNA. The ability of STING to induce type I interferon production lead to studies in the context of antitumor immune response, and as a result, STING has emerged to be a potentially potent target in anti-tumor immunotherapies. A large part of the antitumor effects caused by STING activation may depend upon production of IFN-β by APCs and improved antigen presentation by these cells, which promotes CD8+ T cell priming against tumor-associated antigens. However, STING protein is also expressed broadly in a variety of cell types including myeloid-derived suppressor cells (MDSCs) and cancer cells themselves, in which the function of the pathway has not yet been well characterized (Sokolowska, O. & Nowis, D; STING Signaling in Cancer Cells: Important or Not?; Archivum Immunologiae et Therapiae Experimentalis; Arch. Immunol. Ther. Exp. (2018) 66: 125).

Stimulator of interferon genes (STING), also known as transmembrane protein 173 (TMEM173), mediator of interferon regulatory factor 3 activation (MITA), MPYS or endoplasmic reticulum interferon stimulator (ERIS), is a dimeric protein which is mainly expressed in macrophages, T cells, dendritic cells, endothelial cells, and certain fibroblasts and epithelial cells. STING plays an important role in the innate immune response—mice lacking STING are viable though prone to lethal infection following exposure to a variety of microbes. STING functions as a cytosolic receptor for the second messengers in the form of cytosolic cyclic dinucleotides (CDNs), such as cGAMP and the bacterial second messengers c-di-GMP and c-di-AMP. Upon stimulation by the CDN a conformational change in STING occurs. STING translocates from the ER to the Golgi apparatus and its carboxyterminus is liberated, This leads to the activation of TBK1 (TANK-binding kinase 1)/IRF3 (interferon regulatory factor 3), NF-κB, and STAT6 signal transduction pathways, and thereby promoting type I interferon and proinflammatory cytokine responses. CDNs include canonical cyclic di-GMP (c[G(30-50)pG(30-50)p] or cyclic di-AMP or cyclic GAMP (cGMP-AMP) (Barber, STING-dependent cytosolic DNA sensing pathways; Trends Immunol. 2014 Feb; 35(2):88-93).

CDNs can be exogenously (i.e., bacterially) and/or endogenously produced (i.e., within the host by a host enzyme upon exposure to dsDNA). STING is able to recognize various bacterial second messenger molecules cyclic diguanylate monophosphate (c-di-GMP) and cyclic diadenylate monophosphate (c-di-AMP), which triggers innate immune signaling response (Ma et al., The cGAS-STING Defense Pathway and Its Counteraction by Viruses; Cell Host & Microbe 19, Feb. 10, 2016). Additionally cyclic GMPAMP (cGAMP) can also bind to STING and result inactivation of IRF3 and β-interferon production. Both 3′5′-3′5′ cGAMP (3′3′ cGAMP) produced by Vibrio cholerae, and the metazoan secondary messenger cyclic [G(2′,5′)pA(3′5′)] (2′3′ cGAMP), could activate the innate immune response through STING pathway (Yi et al., Single Nucleotide Polymorphisms of Human STING Can Affect Innate Immune Response to Cyclic Dinucleotides; PLOS One (2013). 8(10)e77846, an references therein). Bacterial and metazoan (e.g., human) c-di-GAMP synthases (cGAS) utilizes GTP and ATP to generate cGAMP capable of STING activation. In contrast to prokaryotic CDNs, which have two canonical 30-50 phosphodiester linkages, the human cGAS product contains a unique 20-50 bond resulting in a mixed linkage cyclic GMP-AMP molecule, denoted as 2′,3′ cGAMP (as described in (Kranzusch et al., Ancient Origin of cGAS-STING Reveals Mechanism of Universal 2′,3′ cGAMP Signaling; Molecular Cell 59, 891-903, Sep. 17, 2015 and references therein). The bacterium Vibrio cholerae encodes an enzyme called DncV that is a structural homolog of cGAS and synthesizes a related second messenger with canonical 3′-5′ bonds (3′,3′ cGAMP).

Components of the stimulator of interferon genes (STING) pathway plays an important role in the detection of tumor cells by the immune system. In preclinical studies, cyclic dinucleotides (CDN), naturally occurring or rationally designed synthetic derivatives, are able to promote an aggressive antitumor response. For example, when co-formulated with an irradiated GM-CSF-secreting whole-cell vaccine in the form of STINGVAX, synthetic CDNs increased the antitumor efficacy and STINGVAX combined with PD-1 blockade induced regression of established tumors (Fu et al., STING agonist formulated cancer vaccines can cure established tumors resistant to PD-1 blockade; Sci Transl Med. 2015 Apr. 15; 7(283): 283ra52). In another example, Smith et al. conducted a study showing that STING agonists may augment CAR T therapy by stimulating the immune response to eliminate tumor cells that are not recognized by the adoptively transferred lymphocytes and thereby improve the effectiveness of CAR T cell therapy (Smith et al., Biopolymers co-delivering engineered T cells and STING agonists can eliminate heterogeneous tumors; J Clin Invest. 2017 Jun. 1; 127(6):2176-2191).

In some embodiments, the genetically engineered bacterium is capable of producing one or more STING agonists. Non limiting examples of STING agonists which can be produced by the genetically engineered bacteria of the disclosure include 3′3′ cGAMP, 2′3′cGAMP, 2′2′-cGAMP, 2′2′-cGAMP VacciGrade™ (Cyclic [G(2′,5′)pA(2′,5′)p]), 2′3′-cGAMP, 2′3′-cGAMP VacciGrade™ (Cyclic [G(2′,5′)pA(3′,5′)p]), 2′3′-cGAM(PS)2 (Rp/Sp), 3′3′-cGAMP, 3′3′-cGAMP VacciGrade™ (Cyclic [G(3′,5′)pA(3′,5′)p]), c-di-AMP, c-di-AMP VacciGrade™ (Cyclic diadenylate monophosphate Th1/Th2 response), 2′3′-c-di-AMP, 2′3′-c-di-AM(PS)2 (Rp,Rp) (Bisphosphorothioate analog of c-di-AMP, Rp isomers), 2′3′-c-di-AM(PS)2 (Rp,Rp) VacciGrade™, c-di-GMP, c-di-GMP VacciGrade™, 2′3′-c-di-GMP, and c-di-IMP. In some embodiments, the genetically engineered bacterium is that comprises a gene encoding one or more enzymes for the production of one or more STING agonists. Cyclic-di-GAMP synthase (cdi-GAMP synthase or cGAS) produces the cyclic-di-GAMP from one ATP and one GTP. In some embodiments, the enzymes are c-di-GAMP synthases (cGAS). In one embodiment, the genetically engineered bacteria comprise one or more gene sequences for the expression of an enzyme in class EC 2.7.7.86. In some embodiments, such enzymes are bacterial enzymes. In some embodiments, the enzyme is a bacterial c-di-GMP synthase. In some embodiments, the enzyme is a bacterial c-GAMP synthase (GMP-AMP synthase). In some embodiments, the bacteria are capable of producing 3′3′ c-dGAMP.

In some embodiments, the bacteria are capable of producing 3′3′-cGAMP. According to the instant disclosure several enzymes suitable for production of 3′3′-cGAMP from genetically engineered bacteria were identified. These enzymes include the Vibrio cholerae cGAS orthologs from Verminephrobacter eiseniae (EF01-2 Earthworm symbiont), Kingella denitrificans (ATCC 33394), and Neisseria bacilliformis (ATCC BAA-1200). Accordingly, in some embodiments, the genetically engineered bacteria comprise gene sequences encoding cGAS from Vibrio cholerae. Accordingly, in some embodiments, the genetically engineered bacteria comprise gene sequences encoding one or more Vibrio cholerae cGAS orthologs from species selected from Verminephrobacter eiseniae (EF01-2 Earthworm symbiont), Kingella denitrificans (ATCC 33394), and Neisseria bacilliformis (ATCC BAA-1200). In some embodiments, the bacteria comprise a gene sequence encoding DncV. In some embodiments, DncV is from Vibrio cholerae. In one embodiment, the DncV orthrolog is from Verminephrobacter eiseniae. In one embodiment, the DncV orthrolog is from Kingella denitrificans. In one embodiment, the DncV orthrolog is from Neisseria bacilliformis. In some embodiments, the genetically engineered bacteria comprise a gene sequence encoding a DncV ortholog from a species selected from Enhydrobacter aerosaccus, Kingella denitrificans, Neisseria bacilliformis, Phaeobacter gallaeciensi, Citromicrobium sp., Roseobacter litoralis, Roseovarius sp., Methylobacterium populi, Erythrobacter sp., Erythrobacter litoralis, Methylophaga thiooxydans, Methylophaga thiooxydans, Herminiimonas arsenicoxydans, Verminephrobacter eiseniae, Methylobacter tundripaludum, Psychrobacter arcticus, Vibrio cholerae, Vibrio sp, Aeromonas salmonicida, Serratia odorifera, Verminephrobacter eiseniae, and Methylovorus glucosetrophus.

In some embodiments, the genetically engineered bacteria are capable of producing 2′3′-cGAMP. Human cGAS is known to produce 2′3′-cGAM P. In some embodiments, the genetically engineered bacteria comprise gene sequences encoding human cGAS.

In some embodiments, the genetically engineered bacteria are capable of increasing c-GAMP (2′3′ or 3′3′) levels in the tumor microenvironment. In some embodiments, the genetically engineered bacteria are capable of increasing c-GAMP levels in the intracellular space In some embodiments, the genetically engineered bacteria are capable of increasing c-GAMP levels inside of a eukaryotic cell. In some embodiments, the genetically engineered bacteria are capable of increasing c-GAMP (2′3′ or 3′3′) levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c-GAMP (2′3′ or 3′3′) inside of a cancer cell. In some embodiments, the genetically engineered bacteria are capable of increasing c-GAMP levels in vitro in the bacterial cell and/or in the growth medium.

In one embodiment, the genetically engineered bacteria comprise gene sequence(s) encoding bacterial c-di-GAMP synthase from Vibrio cholerae. In some embodiments, the enzyme is DncV.

In one embodiment, the genetically engineered bacteria comprise gene sequence(s) encoding c-di-AMP synthase from Verminephrobacter eiseniae. In one embodiment, the bacterial c-di-GAMP synthase is DcnV ortholog from Verminephrobacter eiseniae (EF01-2 Earthworm symbiont). In some embodiments, the genetically engineered bacteria comprise c-di-GAMP synthase gene sequence(s) encoding one or more polypeptide(s) comprising SEQ ID NO: 1262 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1262 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1262. In some specific embodiments, the polypeptide comprises SEQ ID NO: 1262. In other specific embodiments, the polypeptide consists of SEQ ID NO: 1262. In certain embodiments, the bacterial c-di-GAMP synthase gene sequence has at least about 80% identity with SEQ ID NO: 1265. In certain embodiments, the gene sequence has at least about 90% identity with SEQ ID NO: 1265. In certain embodiments, the gene sequence has at least about 95% identity with SEQ ID NO: 1265. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1265. In some specific embodiments, the gene sequence comprises SEQ ID NO: 1265. In other specific embodiments, the gene sequence consists of SEQ ID NO: 1265.

In one embodiment, the genetically engineered bacteria comprise gene sequence(s) encoding c-di-AMP synthase from Kingella denitrificans (ATCC 33394). In one embodiment, the bacterial c-di-GAMP synthase is DcnV ortholog from Kingella denitrificans. In some embodiments, the genetically engineered bacteria comprise c-di-GAMP synthase gene sequence(s) encoding one or more polypeptide(s) comprising SEQ ID NO: 1260 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1260 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1260. In some specific embodiments, the polypeptide comprises SEQ ID NO: 1260. In other specific embodiments, the polypeptide consists of SEQ ID NO: 1260. In certain embodiments, the bacterial c-di-GAMP synthase gene sequence has at least about 80% identity with SEQ ID NO: 1263. In certain embodiments, the gene sequence has at least about 90% identity with SEQ ID NO: 1263. In certain embodiments, the gene sequence has at least about 95% identity with SEQ ID NO: 1263. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1263. In some specific embodiments, the gene sequence comprises SEQ ID NO: 1263. In other specific embodiments, the gene sequence consists of SEQ ID NO: 1263.

In one embodiment, the genetically engineered bacteria comprise gene sequence(s) encoding c-di-AMP synthase from Neisseria bacilliformis (ATCC BAA-1200). In one embodiment, the bacterial c-di-GAMP synthase is DcnV ortholog from Neisseria bacilliformis. In some embodiments, the genetically engineered bacteria comprise c-di-GAMP synthase gene sequence(s) encoding one or more polypeptide(s) comprising SEQ ID NO: 1261 or functional fragments thereof. In some embodiments, genetically engineered bacteria comprise a gene sequence encoding a polypeptide that is at least about 80%, at least about 85%, at least about 90%, at least about 95%, or at least about 99% identity to SEQ ID NO: 1261 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1261. In some specific embodiments, the polypeptide comprises SEQ ID NO: 1261. In other specific embodiments, the polypeptide consists of SEQ ID NO: 1261. In certain embodiments, the c-di-GAMP synthase sequence has at least about 80% identity with SEQ ID NO: 1264. In certain embodiments, the gene sequence has at least about 90% identity with SEQ ID NO: 1264. In certain embodiments, the gene sequence has at least about 95% identity with SEQ ID NO: 1264. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1264. In some specific embodiments, the gene sequence comprises SEQ ID NO: 1264. In other specific embodiments, the gene sequence consists of SEQ ID NO: 1264.

In one embodiment, the genetically engineered bacteria comprise gene sequence(s) encoding mammalian c-di-GAMP enzymes. In some embodiments, the STING agonist producing enzymes are human enzymes. In some embodiments, the gene sequence(s) are codon-optimized for expression in a microorganism host cell. In one embodiment, the genetically engineered bacteria comprise gene sequence(s) encoding the human polypeptide cGAS. In some embodiments, the genetically engineered bacteria comprise human cGAS gene sequence(s) encoding one or more polypeptide(s) comprising SEQ ID NO: 1254 or functional fragments thereof. In some embodiments, genetically engineered bacteria comprise a gene sequence encoding a polypeptide that is at least about 80%, at least about 85%, at least about 90%, at least about 95%, or at least about 99% identity to SEQ ID NO: 1254 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1254. In some specific embodiments, the polypeptide comprises SEQ ID NO: 1254. In other specific embodiments, the polypeptide consists of SEQ ID NO: 1254. In certain embodiments, the human cGAS sequence has at least about 80% identity with SEQ ID NO: 1255. In certain embodiments, the gene sequence has at least about 90% identity with SEQ ID NO: 1255. In certain embodiments, the gene sequence has at least about 95% identity with SEQ ID NO: 1255. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1255. In some specific embodiments, the gene sequence comprises SEQ ID NO: 1264. In other specific embodiments, the gene sequence consists of SEQ ID NO: 1255.

In some embodiments, the bacteria are capable of producing cyclic-di-GMP. Accordingly, in some embodiments, the genetically engineered bacteria comprise gene sequence(s) encoding one or more diguanylate cyclase(s).

In some embodiments, the genetically engineered bacteria are capable of increasing cyclic-di-GMP levels in the tumor microenvironment. In some embodiments, the genetically engineered bacteria are capable of increasing cyclic-di-GMP levels in the intracellular space In some embodiments, the genetically engineered bacteria are capable of increasing cyclic-di-GMP levels inside of a eukaryotic cell. In some embodiments, the genetically engineered bacteria are capable of increasing cyclic-di-GMP levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c cyclic-di-GMP levels inside of a cancer cell. In some embodiments, the genetically engineered bacteria are capable of increasing c-GMP levels in vitro in the bacterial cell and/or in the growth medium.

In some embodiments, the genetically engineered bacteria are capable of producing c-diAMP. Diadenylate cyclase produces one molecule cyclic-di-AMP from two ATP molecules. In one embodiment, the genetically engineered bacteria comprise one or more gene sequences for the expression of a diadenylate cyclase. In one embodiment, the genetically engineered bacteria comprise one or more gene sequences for the expression of an enzyme in class EC 2.7.7.85. In one embodiment, the diadenylate cyclase is a bacterial diadenylate cyclase. In one embodiment, the diadenylate cyclase is DacA. In one embodiment, the DacA is from Listeria monocytogenes.

In some embodiments, the genetically engineered bacteria comprise DacA gene sequence(s) encoding one or more polypeptide(s) comprising SEQ ID NO: 1257 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1257 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1257. In some specific embodiments, the polypeptide comprises SEQ ID NO: 1257. In other specific embodiments, the polypeptide consists of SEQ ID NO: 1257. In certain embodiments, the Dac A sequence has at least about 80% identity with SEQ ID NO: 1258. In certain embodiments, the gene sequence has at least about 90% identity with SEQ ID NO: 1258. In certain embodiments, the gene sequence has at least about 95% identity with SEQ ID NO: 1258. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1258. In some specific embodiments, the gene sequence comprises SEQ ID NO: 1258. In other specific embodiments, the gene sequence consists of SEQ ID NO: 1258.

In some embodiments, the genetically engineered bacteria comprise DacA gene sequence(s) operably linked to a promoter which is inducible under low oxygen conditions, e.g., an FNR inducible promoter as described herein. In certain embodiments, the sequence of the DacA gene operably linked to the FNR inducible promoter has at least about 80% identity with SEQ ID NO: 1284. In certain embodiments, the sequence of the DacA gene operably linked to the FNR inducible promoter has at least about 90% identity with SEQ ID NO: 1258. In certain embodiments, the sequence of the DacA gene operably linked to the FNR inducible promoter has at least about 95% identity with SEQ ID NO: 1258. In some embodiments, the sequence of the DacA gene operably linked to the FNR inducible promoter has at least about 85%, 86%, 87%, 88%, 89%, 90%, 91%, 92%, 93%, 94%, 95%, 96%, 97%, 98%, or 99% identity with SEQ ID NO: 1258. In some specific embodiments, the sequence of the DacA gene operably linked to the FNR inducible promoter comprises SEQ ID NO: 1258. In other specific embodiments the sequence of the DacA gene operably linked to the FNR inducible promoter consists of SEQ ID NO: 1258.

Other suitable diadenylate cyclases are known in the art and include those include in the EggNog database (http://eggnogdb.embl.de). Non-limiting examples of diadenylate cyclases which can be expressed by the bacteria include Megasphaera sp. UPII 135-E (HMPREF1040_0026), Streptococcus anginosus SK52=DSM 20563 (HMPREF9966_0555), Streptococcus mitis by. 2 str. SK95 (HMPREF9965_1675), Streptococcus infantis SK1076 (HMPREF9967_1568), Acetonema longum DSM 6540 (ALO_03356), Sporosarcina newyorkensis 2681 (HMPREF9372_2277), Listeria monocytogenes str. Scott A (BN418_2551), Candidatus Arthromitus sp. SFB-mouse-Japan (SFBM_1354), Haloplasma contractile SSD-17B 2 seqs HLPCO_01750, HLPCO_08849), Lactobacillus kefiranofaciens ZW3 (WANG_0941), Mycoplasma anatis 1340 (GIG_03148), Streptococcus constellatus subsp. pharyngis SK1060=CCUG 46377 (HMPREF1042_1168), Streptococcus infantis SK970 (HMPREF9954_1628), Paenibacillus mucilaginosus KNP414 (YBBP), Nostoc sp. PCC 7120 (ALL2996), Mycoplasma columbinum SF7 (MCSF7_01321), Lactobacillus ruminis SPM0211 (LRU_01199), Candidatus Arthromitus sp. SFB-rat-Yit (RATSFB_1182), Clostridium sp. SY8519 (CXIVA_02190), Brevibacillus laterosporus LMG 15441 (BRLA_CO2240), Weissella koreensis KACC 15510 (WKK_01955), Brachyspira intermedia PWS/A (BINT_2204), Bizionia argentinensis JUB59 (BZARG_2617), Streptococcus salivarius 57.1 (SSAL_01348), Alicyclobacillus acidocaldarius subsp. acidocaldarius Tc-4-1 (TC41_3001), Sulfobacillus acidophilus TPY (TPY_0875), Streptococcus pseudopneumoniae IS7493 (SPPN_07660), Megasphaera elsdenii DSM 20460 (MELS_0883), Streptococcus infantarius subsp. infantarius CJ18 (SINF_1263), Blattabacterium sp. (Mastotermes darwiniensis) str. MADAR (MADAR_511), Blattabacterium sp. (Cryptocercus punctulatus) str. Cpu (BLBCPU_093), Synechococcus sp. CC9605 (SYNCC9605_1630), Thermus sp. CCB_US3_UF1 (AEV17224.1), Mycoplasma haemocanis str. Illinois (MHC_04355), Streptococcus macedonicus ACA-DC 198 (YBBP), Mycoplasma hyorhinis GDL-1 (MYM_0457), Synechococcus elongatus PCC 7942 (SYNPCC7942_0263), Synechocystis sp. PCC 6803 (SLL0505), Chlamydophila pneumoniae CWL029 (YBBP), Microcoleus chthonoplastes PCC 7420 (MC7420_6818), Persephonella marina EX-H1 (PERMA_1676), Desulfitobacterium hafniense Y51 (DSY4489), Prochlorococcus marinus str. AS9601 (A9601_11971), Flavobacteria bacterium BBFL7 (BBFL7_02553), Sphaerochaeta globus str. Buddy (SPIBUDDY_2293), Sphaerochaeta pleomorpha str. Grapes (SPIGRAPES_2501), Staphylococcus aureus subsp. aureus Mu50 (SAV2163), Streptococcus pyogenes M1 GAS (SPY_1036), Synechococcus sp. WH 8109 (SH8109_2193), Prochlorococcus marinus subsp. marinus str. CCMP1375 (PRO_1104), Prochlorococcus marinus str. MIT 9515 (P9515_11821), Prochlorococcus marinus str. MIT 9301 (P9301_11981), Prochlorococcus marinus str. NATL1A (NATL1_14891), Listeria monocytogenes EGD-e (LMO2120), Streptococcus pneumoniae TIGR4 2 seqs SPNET_02000368, SP_1561), Streptococcus pneumoniae R6 (SPR1419), Staphylococcus epidermidis RP62A (SERP1764), Staphylococcus epidermidis ATCC 12228 (SE_1754), Desulfobacterium autotrophicum HRM2 (HRM2_32880), Desulfotalea psychrophila LSv54 (DP1639), Cyanobium sp. PCC 7001 (CPCC7001_1029), Chlamydophila pneumoniae TW-183 (YBBP), Leptospira interrogans serovar Lai str. 56601 (LA_3304), Clostridium perfringens ATCC 13124 (CPF_2660), Thermosynechococcus elongatus BP-1 (TLR1762), Bacillus anthracis str. Ames (BA_0155), Clostridium thermocellum ATCC 27405 (CTHE_1166), Leuconostoc mesenteroides subsp. mesenteroides ATCC 8293 (LEUM_1568), Oenococcus oeni PSU-1 (OEOE_1656), Trichodesmium erythraeum IMS101 (TERY_2433), Tannerella forsythia ATCC 43037 (BFO_1347), Sulfurihydrogenibium azorense Az-Fu1 (SULAZ_1626), Candidatus Koribacter versatilis Ellin345 (ACID345_0278), Desulfovibrio alaskensis G20 (DDE_1515), Carnobacterium sp. 17-4 (YBBP), Streptococcus mutans UA159 (SMU_1428 C), Mycoplasma agalactiae (MAG3060), Streptococcus agalactiae NEM316 (GBS0902), Clostridium tetani E88 (CTC_02549), Ruminococcus champanellensis 18P13 (RUM_14470), Croceibacter atlanticus HTCC2559 (CA2559_13513), Streptococcus uberis 0140J (SUB1092), Chlamydophila abortus S26/3 (CAB642), Lactobacillus plantarum WCFS1 (LP_0818), Oceanobacillus iheyensis HTE831 (OB0230), Synechococcus sp. RS9916 (RS9916_31367), Synechococcus sp. RS9917 (RS9917_00967), Bacillus subtilis subsp. subtilis str. 168 (YBBP), Aquifex aeolicus VF5 (AQ_1467), Borrelia burgdorferi B31 (BB_0008), Enterococcus faecalis V583 (EF_2157), Bacteroides thetaiotaomicron VPI-5482 (BT_3647), Bacillus cereus ATCC 14579 (BC_0186), Chlamydophila caviae GPIC (CCA_00671), Synechococcus sp. CB0101 (SCB01_010100000902), Synechococcus sp. CB0205 (SCB02_010100012692), Candidatus Solibacter usitatus Ellin6076 (ACID_1909), Geobacillus kaustophilus HTA426 (GKO152), Verrucomicrobium spinosum DSM 4136 (VSPID_010100022530), Anabaena variabilis ATCC 29413 (AVA_0913), Porphyromonas gingivalis W83 (PG_1588), Chlamydia muridarum Nigg (TC_0280), Deinococcus radiodurans R1 (DR_0007), Geobacter sulfurreducens PCA 2 seqs GSU1807, GSU0868), Mycoplasma arthritidis 158L3-1 (MARTH_ORF527), Mycoplasma genitalium G37 (MG105), Treponema denticola ATCC 35405 (TDE_1909), Treponema pallidum subsp. pallidum str. Nichols (TP_0826), butyrate-producing bacterium SS3/4 (CK3_23050), Carboxydothermus hydrogenoformans Z-2901 (CHY_2015), Ruminococcus albus 8 (CUS_5386), Streptococcus mitis NCTC 12261 (SM12261_1151), Gloeobacter violaceus PCC 7421 (GLL0109), Lactobacillus johnsonii NCC 533 (LJ_0892), Exiguobacterium sibiricum 255-15 (EXIG_0138), Mycoplasma hyopneumoniae J (MHJ_0485), Mycoplasma synoviae 53 (MS53_0498), Thermus thermophilus HB27 (TT_C1660), Onion yellows phytoplasma OY-M (PAM_584), Streptococcus thermophilus LMG 18311 (OSSG), Candidatus Protochlamydia amoebophila UWE25 (PC1633), Chlamydophila felis Fe/C-56 (CF0340), Bdellovibrio bacteriovorus HD100 (BD1929), Prevotella ruminicola 23 (PRU_2261), Moorella thermoacetica ATCC 39073 (MOTH_2248), Leptospira interrogans serovar Copenhageni str. Fiocruz L1-130 (LIC_10844), Mycoplasma mobile 163K (MMOB4550), Synechococcus elongatus PCC 6301 (SYC1250_C), Cytophaga hutchinsonii ATCC 33406 (CHU_3222), Geobacter metallireducens GS-15 2 seqs GMET_1888, GMET_1168), Bacillus halodurans C-125 (BH0265), Bacteroides fragilis NCTC 9343 (BF0397), Chlamydia trachomatis D/UW-3/CX (YBBP), Clostridium acetobutylicum ATCC 824 (CA_C3079), Clostridium difficile 630 (CD0110), Lactobacillus acidophilus NCFM (LBA0714), Lactococcus lactis subsp. lactis 111403 (YEDA), Listeria innocua Clip11262 (L1N2225), Mycoplasma penetrans HF-2 (MYPE2120), Mycoplasma pulmonis UAB CTIP (MYPU_4070), Thermoanaerobacter tengcongensis MB4 (TTE2209), Pediococcus pentosaceus ATCC 25745 (PEPE_0475), Bacillus licheniformis DSM 13=ATCC 14580 2 seqs YBBP, BL02701), Staphylococcus haemolyticus JCSC1435 (SH0877), Desulfuromonas acetoxidans DSM 684 (DACE_0543), Thermodesulfovibrio yellowstonii DSM 11347 (THEYE_A0044), Mycoplasma bovis PG45 (MBOVPG45_0394), Anaeromyxobacter dehalogenans 2CP-C(ADEH_1497), Clostridium beijerinckii NCIMB 8052 (CBEI_0200), Borrelia garinii PBi (BG0008), Symbiobacterium thermophilum IAM 14863 (STH192), Alkaliphilus metalliredigens QYMF (AMET_4313), Thermus thermophilus HB8 (TTHA0323), Coprothermobacter proteolyticus DSM 5265 (COPRO5265_1086), Thermomicrobium roseum DSM 5159 (TRD_0688), Salinibacter ruber DSM 13855 (SRU_1946), Dokdonia donghaensis MED134 (MED134_03354), Polaribacter irgensii 23-P (P123P_01632), Psychroflexus torquis ATCC 700755 (P700755_02202), Robiginitalea biformata HTCC2501 (RB2501_10597), Polaribacter sp. MED152 (MED152_11519), Maribacter sp. HTCC2170 (FB2170_01652), Microscilla marina ATCC 23134 (M23134_07024), Lyngbya sp. PCC 8106 (L8106_18951), Nodularia spumigena CCY9414 (N9414_23393), Synechococcus sp. BL107 (BL107_11781), Bacillus sp. NRRL B-14911 (B14911_19485), Lentisphaera araneosa HTCC2155 (LNTAR_18800), Lactobacillus sakei subsp. sakei 23K (LCA_1359), Mariprofundus ferrooxydans PV-1 (SPV1_13417), Borrelia hermsii DAH (BH0008), Borrelia turicatae 91E135 (BT0008), Bacillus weihenstephanensis KBAB4 (BCERKBAB4_0149), Bacillus cytotoxicus NVH 391-98 (BCER98_0148), Bacillus pumilus SAFR-032 (YBBP), Geobacter sp. FRC-32 2 seqs GEOB_2309, GEOB_3421), Herpetosiphon aurantiacus DSM 785 (HAUR_3416), Synechococcus sp. RCC307 (SYNRCC307_0791), Synechococcus sp. CC9902 (SYNCC9902_1392), Deinococcus geothermalis DSM 11300 (DGEO_0135), Synechococcus sp. PCC 7002 (SYNPCC7002_A0098), Synechococcus sp. WH 7803 (SYNWH7803_1532), Pedosphaera parvula Ellin514 (CFLAV_PD5552), Synechococcus sp. JA-3-3Ab (CYA_2894), Synechococcus sp. JA-2-3Ba(2-13) (CYB_1645), Aster yellows witches-broom phytoplasma AYWB (AYWB_243), Paenibacillus sp. JDR-2 (PJDR2_5631), Chloroflexus aurantiacus J-10-fl (CAUR_1577), Lactobacillus gasseri ATCC 33323 (LGAS_1288), Bacillus amyloliquefaciens FZB42 (YBBP), Chloroflexus aggregans DSM 9485 (CAGG_2337), Acaryochloris marina MBIC11017 (AM1_0413), Blattabacterium sp. (Blattella germanica) str. Bge (BLBBGE_101), Simkania negevensis Z (YBBP), Chlamydophila pecorum E58 (G5S_1046), Chlamydophila psittaci 6BC 2 seqs CPSIT_0714, G50_0707), Carnobacterium sp. AT7 (CAT7_06573), Finegoldia magna ATCC 29328 (FMG_1225), Syntrophomonas wolfei subsp. wolfei str. Goettingen (SWOL_2103), Syntrophobacter fumaroxidans MPOB (SFUM_3455), Pelobacter carbinolicus DSM 2380 (PCAR_0999), Pelobacter propionicus DSM 2379 2 seqs PPRO_2640, PPRO_2254), Thermoanaerobacter pseudethanolicus ATCC 33223 (TETH39_0457), Victivallis vadensis ATCC BAA-548 (VVAD_PD2437), Staphylococcus saprophyticus subsp. saprophyticus ATCC 15305 (SSP0722), Bacillus coagulans 36D1 (BCOA_1105), Mycoplasma hominis ATCC 23114 (MHO_0510), Lactobacillus reuteri 100-23 (LREU23DRAFT_3463), Desulfotomaculum reducens MI-1 (DRED_0292), Leuconostoc citreum KM20 (LCK_01297), Paenibacillus polymyxa E681 (PPE_04217), Akkermansia muciniphila ATCC BAA-835 (AMUC_0400), Alkaliphilus oremlandii OhILAs (CLOS_2417), Geobacter uraniireducens Rf4 2 seqs GURA_1367, GURA_2732), Caldicellulosiruptor saccharolyticus DSM 8903 (CSAC_1183), Pyramidobacter piscolens W5455 (HMPREF7215_0074), Leptospira borgpetersenii serovar Hardjo-bovis L550 (LBL_0913), Roseiflexus sp. RS-1 (ROSERS_1145), Clostridium phytofermentans ISDg (CPHY_3551), Brevibacillus brevis NBRC 100599 (BBR47_02670), Exiguobacterium sp. AT1b (EAT1B_1593), Lactobacillus salivarius UCC118 (LSL_1146), Lawsonia intracellularis PHE/MN1-00 (110190), Streptococcus mitis B6 (SMI_1552), Pelotomaculum thermopropionicum SI (PTH_0536), Streptococcus pneumoniae D39 (SPD_1392), Candidatus Phytoplasma mali (ATP_00312), Gemmatimonas aurantiaca T-27 (GAU_1394), Hydrogenobaculum sp. YO4AAS1 (HY04AAS1_0006), Roseiflexus castenholzii DSM 13941 (RCAS_3986), Listeria welshimeri serovar 6b str. SLCC5334 (LWE2139), Clostridium novyi NT (NT01CX_1162), Lactobacillus brevis ATCC 367 (LVIS_0684), Bacillus sp. B14905 (BB14905_08668), Algoriphagus sp. PR1 (ALPR1_16059), Streptococcus sanguinis SK36 (SSA_0802), Borrelia afzelii PKo 2 seqs BAPKO_0007, AEL69242.1), Lactobacillus delbrueckii subsp. bulgaricus ATCC 11842 (LDB0651), Streptococcus suis 05ZYH33 (SSU05_1470), Kordia algicida OT-1 (KAOT1_10521), Pedobacter sp. BAL39 (PBAL39_03944), Flavobacteriales bacterium ALC-1 (FBALC1_04077), Cyanothece sp. CCY0110 (CY0110_30633), Plesiocystis pacifica SIR-1 (PPSIR1_10140), Clostridium cellulolyticum H10 (CCEL_1201), Cyanothece sp. PCC 7425 (CYAN7425_4701), Staphylococcus carnosus subsp. carnosus TM300 (SCA_1665), Bacillus pseudofirmus OF4 (YBBP), Leeuwenhoekiella blandensis MED217 (MED217_04352), Geobacter lovleyi SZ 2 seqs GLOV_3055, GLOV_2524), Streptococcus equi subsp. zooepidemicus (SEZ_1213), Thermosinus carboxydivorans Nor1 (TCARDRAFT_1045), Geobacter bemidjiensis Bem (GBEM_0895), Anaeromyxobacter sp. Fw109-5 (ANAE109_2336), Lactobacillus helveticus DPC 4571 (LHV_0757), Bacillus sp. m3-13 (BM3-1_010100010851), Gramella forsetii KT0803 (GFO_0428), Ruminococcus obeum ATCC 29174 (RUMOBE_03597), Ruminococcus torques ATCC 27756 (RUMTOR_00870), Dorea formicigenerans ATCC 27755 (DORFOR_00204), Dorea longicatena DSM 13814 (DORLON_01744), Eubacterium ventriosum ATCC 27560 (EUBVEN_01080), Desulfovibrio piger ATCC 29098 (DESPIG_01592), Parvimonas micra ATCC 33270 (PEPMIC_01312), Pseudoflavonifractor capillosus ATCC 29799 (BACCAP_01950), Clostridium scindens ATCC 35704 (CLOSCI_02389), Eubacterium hallii DSM 3353 (EUBHAL_01228), Ruminococcus gnavus ATCC 29149 (RUMGNA_03537), Subdoligranulum variabile DSM 15176 (SUBVAR_05177), Coprococcus eutactus ATCC 27759 (COPEUT_01499), Bacteroides ovatus ATCC 8483 (BACOVA_03480), Parabacteroides merdae ATCC 43184 (PARMER_03434), Faecalibacterium prausnitzii A2-165 (FAEPRAA2165_01954), Clostridium sp. L2-50 (CLOL250_00341), Anaerostipes caccae DSM 14662 (ANACAC_00219), Bacteroides caccae ATCC 43185 (BACCAC_03225), Clostridium bolteae ATCC BAA-613 (CLOBOL_04759), Borrelia duttonii Ly (BDU_14), Cyanothece sp. PCC 8801 (PCC8801_0127), Lactococcus lactis subsp. cremoris MG1363 (LLMG_0448), Geobacillus thermodenitrificans NG80-2 (GTNG_0149), Epulopiscium sp. N.t. morphotype B (EPULO_010100003839), Lactococcus garvieae Lg2 (LCGL_0304), Clostridium leptum DSM 753 (CLOLEP_03097), Clostridium spiroforme DSM 1552 (CLOSPI_01608), Eubacterium dolichum DSM 3991 (EUBDOL_00188), Clostridium kluyveri DSM 555 (CKL_0313), Porphyromonas gingivalis ATCC 33277 (PGN_0523), Bacteroides vulgatus ATCC 8482 (BVU_0518), Parabacteroides distasonis ATCC 8503 (BDI_3368), Staphylococcus hominis subsp. hominis C80 (HMPREF0798_01968), Staphylococcus caprae C87 (HMPREF0786_02373), Streptococcus sp. C150 (HMPREF0848_00423), Sulfurihydrogenibium sp. YO3AOP1 (SYO3AOP1_0110), Desulfatibacillum alkenivorans AK-01 (DALK_0397), Bacillus selenitireducens MLS10 (BSEL_0372), Cyanothece sp. ATCC 51142 (CCE_1350), Lactobacillus jensenii 1153 (LBJG_01645), Acholeplasma laidlawii PG-8A (ACL_1368), Bacillus coahuilensis m4-4 (BCOAM_010100001120), Geobacter sp. M18 2 seqs GM18_0792, GM18_2516), Lysinibacillus sphaericus C3-41 (BSPH_4568), Clostridium botulinum NCTC 2916 (CBN_3506), Clostridium botulinum C str. Eklund (CBC_A1575), Alistipes putredinis DSM 17216 (ALIPUT_00190), Anaerofustis stercorihominis DSM 17244 (ANASTE_01539), Anaerotruncus colihominis DSM 17241 (ANACOL_02706), Clostridium bartlettii DSM 16795 (CLOBAR_00759), Clostridium ramosum DSM 1402 (CLORAM_01482), Borrelia valaisiana VS116 (BVAVS116_0007), Sorangium cellulosum So ce 56 (SCE7623), Microcystis aeruginosa NIES-843 (MAE_25390), Bacteroides stercoris ATCC 43183 (BACSTE_02634), Candidatus Amoebophilus asiaticus 5a2 (AASI_0652), Leptospira biflexa serovar Patoc strain Patoc 1 (Paris) (LEPBI_I0735), Clostridium sp. 7_2_43 FAA (CSBG_00101), Desulfovibrio sp. 3_1_syn3 (HMPREF0326_02254), Ruminococcus sp. 5_1_39 BFAA (RSAG_02135), Clostridiales bacterium 1_7_47 FAA (CBFG_00347), Bacteroides fragilis 3_1_12 (BFAG_02578), Natranaerobius thermophilus JW/NM-WN-LF (NTHER_0240), Macrococcus caseolyticus JCSC5402 (MCCL_0321), Streptococcus gordonii str. Challis substr. CH1 (SGO_0887), Dethiosulfovibrio peptidovorans DSM 11002 (DPEP_2062), Coprobacillus sp. 29_1 (HMPREF9488_03448), Bacteroides coprocola DSM 17136 (BACCOP_03665), Coprococcus comes ATCC 27758 (COPCOM_02178), Geobacillus sp. WCH70 (GWCH70_0156), uncultured Termite group 1 bacterium phylotype Rs-D17 (TGRD_209), Dyadobacter fermentans DSM 18053 (DFER_0224), Bacteroides intestinalis DSM 17393 (BACINT_00700), Ruminococcus lactaris ATCC 29176 (RUMLAC_01257), Blautia hydrogenotrophica DSM 10507 (RUMHYD_01218), Candidatus Desulforudis audaxviator MP104C (DAUD_1932), Marvinbryantia formatexigens DSM 14469 (BRYFOR_07410), Sphaerobacter thermophilus DSM 20745 (STHE_1601), Veillonella parvula DSM 2008 (VPAR_0292), Methylacidiphilum infernorum V4 (MINF_1897), Paenibacillus sp. Y412MC10 (GYMC10_5701), Bacteroides finegoldii DSM 17565 (BACFIN_07732), Bacteroides eggerthii DSM 20697 (BACEGG_03561), Bacteroides pectinophilus ATCC 43243 (BACPEC_02936), Bacteroides plebeius DSM 17135 (BACPLE_00693), Desulfohalobium retbaense DSM 5692 (DRET_1725), Desulfotomaculum acetoxidans DSM 771 (DTOX_0604), Pedobacter heparinus DSM 2366 (PHEP_3664), Chitinophaga pinensis DSM 2588 (CPIN_5466), Flavobacteria bacterium MS024-2A (FLAV2ADRAFT_0090), Flavobacteria bacterium MS024-3C (FLAV3CDRAFT_0851), Moorea producta 3L (LYNGBM3L_14400), Anoxybacillus flavithermus WK1 (AFLV_0149), Mycoplasma fermentans PG18 (MBIO_0474), Chthoniobacter flavus Ellin428 (CFE428DRAFT_3031), Cyanothece sp. PCC 7822 (CYAN7822_1152), Borrelia spielmanii A14S (BSPA14S_0009), Heliobacterium modesticaldum Ice1 (HM1_1522), Thermus aquaticus Y51MC23 (TAQDRAFT_3938), Clostridium sticklandii DSM 519 (CLOST_0484), Tepidanaerobacter sp. Re1 (TEPRE1_0323), Clostridium hiranonis DSM 13275 (CLOHIR_00003), Mitsuokella multacida DSM 20544 (MITSMUL_03479), Haliangium ochraceum DSM 14365 (HOCH_3550), Spirosoma linguale DSM 74 (SLIN_2673), unidentified eubacterium SCB49 (SCB49_03679), Acetivibrio cellulolyticus CD2 (ACELC_020100013845), Lactobacillus buchneri NRRL B-30929 (LBUC_1299), Butyrivibrio crossotus DSM 2876 (BUTYVIB_02056), Candidatus Azobacteroides pseudotrichonymphae genomovar. CFP2 (CFPG_066), Mycoplasma crocodyli MP145 (MCRO_0385), Arthrospira maxima CS-328 (AMAXDRAFT_4184), Eubacterium eligens ATCC 27750 (EUBELI_01626), Butyrivibrio proteoclasticus B316 (BPR_I2587), Chloroherpeton thalassium ATCC 35110 (CTHA_1340), Eubacterium biforme DSM 3989 (EUBIFOR_01794), Rhodothermus marinus DSM 4252 (RMAR_0146), Borrelia bissettii DN127 (BBIDN127_0008), Capnocytophaga ochracea DSM 7271 (COCH_2107), Alicyclobacillus acidocaldarius subsp. acidocaldarius DSM 446 (AACI_2672), Caldicellulosiruptor bescii DSM 6725 (ATHE_0361), Denitrovibrio acetiphilus DSM 12809 (DACET_1298), Desulfovibrio desulfuricans subsp. desulfuricans str. ATCC 27774 (DDES_1715), Anaerococcus lactolyticus ATCC 51172 (HMPREF0072_1645), Anaerococcus tetradius ATCC 35098 (HMPREF0077_0902), Finegoldia magna ATCC 53516 (HMPREF0391_10377), Lactobacillus antri DSM 16041 (YBBP), Lactobacillus buchneri ATCC 11577 (HMPREF0497_2752), Lactobacillus ultunensis DSM 16047 (HMPREF0548_0745), Lactobacillus vaginalis ATCC 49540 (HMPREF0549_0766), Listeria grayi DSM 20601 (HMPREF0556_11652), Sphingobacterium spiritivorum ATCC 33861 (HMPREF0766_11787), Staphylococcus epidermidis M23864:W1 (HMPREF0793_0092), Streptococcus equinus ATCC 9812 (HMPREF0819_0812), Desulfomicrobium baculatum DSM 4028 (DBAC_0255), Thermanaerovibrio acidaminovorans DSM 6589 (TACI_0837), Thermobaculum terrenum ATCC BAA-798 (TTER_1817), Anaerococcus prevotii DSM 20548 (APRE_0370), Desulfovibrio salexigens DSM 2638 (DESAL_1795), Brachyspira murdochii DSM 12563 (BMUR_2186), Meiothermus silvanus DSM 9946 (MESIL_0161), Bacillus cereus Rock4-18 (BCERE0024_1410), Cylindrospermopsis raciborskii CS-505 (CRC_01921), Raphidiopsis brookii D9 (CRD_01188), Clostridium carboxidivorans P7 2 seqs CLCAR_0016, CCARBDRAFT_4266), Clostridium botulinum E1 str. BoNT E Beluga (CLO_3490), Blautia hansenii DSM 20583 (BLAHAN_07155), Prevotella copri DSM 18205 (PREVCOP_04867), Clostridium methylpentosum DSM 5476 (CLOSTMETH_00084), Lactobacillus casei BL23 (LCABL_11800), Bacillus megaterium QM B1551 (BMQ_0195), Treponema primitia ZAS-2 (TREPR_1936), Treponema azotonutricium ZAS-9 (TREAZ_0147), Holdemania filiformis DSM 12042 (HOLDEFILI_03810), Filifactor alocis ATCC 35896 (HMPREF0389_00366), Gemella haemolysans ATCC 10379 (GEMHA0001_0912), Selenomonas sputigena ATCC 35185 (SELSP_1610), Veillonella dispar ATCC 17748 (VEIDISOL_01845), Deinococcus deserti VCD115 (DEIDE_19700), Bacteroides coprophilus DSM 18228 (BACCOPRO_00159), Nostoc azollae 0708 (AAZO_4735), Erysipelotrichaceae bacterium 5_2_54 FAA (HMPREF0863_02273), Ruminococcaceae bacterium D16 (HMPREF0866_01061), Prevotella bivia JCVIHMP010 (HMPREF0648_0338), Prevotella melaninogenica ATCC 25845 (HMPREF0659_A6212), Porphyromonas endodontalis ATCC 35406 (POREN0001_0251), Capnocytophaga sputigena ATCC 33612 (CAPSP0001_0727), Capnocytophaga gingivalis ATCC 33624 (CAPGI0001_1936), Clostridium hylemonae DSM 15053 (CLOHYLEM_04631), Thermosediminibacter oceani DSM 16646 (TOCE_1970), Dethiobacter alkaliphilus AHT 1 (DEALDRAFT_0231), Desulfonatronospira thiodismutans ASO3-1 (DTHIO_PD2806), Clostridium sp. D5 (HMPREF0240_03780), Anaerococcus hydrogenalis DSM 7454 (ANHYDRO_01144), Kyrpidia tusciae DSM 2912 (BTUS_0196), Gemella haemolysans M341 (HMPREF0428_01429), Gemella morbillorum M424 (HMPREF0432_01346), Gemella sanguinis M325 (HMPREF0433_01225), Prevotella oris C735 (HMPREF0665_01741), Streptococcus sp. M143 (HMPREF0850_00109), Streptococcus sp. M334 (HMPREF0851_01652), Bilophila wadsworthia 3_1_6 (HMPREF0179_00899), Brachyspira hyodysenteriae WA1 (BHWA1_01167), Enterococcus gallinarum EG2 (EGBG_00820), Enterococcus casseliflavus EC20 (ECBG_00827), Enterococcus faecium C68 (EFXG_01665), Syntrophus aciditrophicus SB (SYN_02762), Lactobacillus rhamnosus GG 2 seqs OSSG, LRHM_0937), Acidaminococcus intestini RyC-MR95 (ACIN_2069), Mycoplasma conjunctivae HRC/581 (MCJ_002940), Halanaerobium praevalens DSM 2228 (HPRAE_1647), Aminobacterium colombiense DSM 12261 (AMICO_0737), Clostridium cellulovorans 743B (CLOCEL_3678), Desulfovibrio magneticus RS-1 (DMR_25720), Spirochaeta smaragdinae DSM 11293 (SPIRS_1647), Bacteroidetes oral taxon 274 str. F0058 (HMPREF0156_01826), Lachnospiraceae oral taxon 107 str. F0167 (HMPREF0491_01238), Lactobacillus coleohominis 101-4-CHN (HMPREF0501_01094), Lactobacillus jensenii 27-2-CHN (HMPREF0525_00616), Prevotella buccae D17 (HMPREF0649_02043), Prevotella sp. oral taxon 299 str. F0039 (HMPREF0669_01041), Prevotella sp. oral taxon 317 str. F0108 (HMPREF0670_02550), Desulfobulbus propionicus DSM 2032 2 seqs DESPR_2503, DESPR_1053), Thermoanaerobacterium thermosaccharolyticum DSM 571 (TTHE_0484), Thermoanaerobacter italicus Ab9 (THIT_1921), Thermovirga lienii DSM 17291 (TLIE_0759), Aminomonas paucivorans DSM 12260 (APAU_1274), Streptococcus mitis SK321 (SMSK321_0127), Streptococcus mitis SK597 (SMSK597_0417), Roseburia hominis A2-183 (RHOM_12405), Oribacterium sinus F0268 (HMPREF6123_0887), Prevotella bergensis DSM 17361 (HMPREF0645_2701), Selenomonas noxia ATCC 43541 (YBBP), Weissella paramesenteroides ATCC 33313 (HMPREF0877_0011), Lactobacillus amylolyticus DSM 11664 (HMPREF0493_1017), Bacteroides sp. D20 (HMPREF0969_02087), Clostridium papyrosolvens DSM 2782 (CPAP_3968), Desulfurivibrio alkaliphilus AHT2 (DAAHT2_0445), Acidaminococcus fermentans DSM 20731 (ACFER_0601), Abiotrophia defectiva ATCC 49176 (GCWU000182_00063), Anaerobaculum hydrogeniformans ATCC BAA-1850 (HMPREF1705_01115), Catonella morbi ATCC 51271 (GCWU000282_00629), Clostridium botulinum D str. 1873 (CLG_B1859), Dialister invisus DSM 15470 (GCWU000321_01906), Fibrobacter succinogenes subsp. succinogenes S85 2 seqs FSU_0028, FISUC_2776), Desulfovibrio fructosovorans JJ (DESFRDRAFT_2879), Peptostreptococcus stomatis DSM 17678 (HMPREF0634_0727), Staphylococcus warneri L37603 (STAWA0001_0094), Treponema vincentii ATCC 35580 (TREVI0001_1289), Porphyromonas uenonis 60-3 (PORUE0001_0199), Peptostreptococcus anaerobius 653-L (HMPREF0631_1228), Peptoniphilus lacrimalis 315-B (HMPREF0628_0762), Candidatus Phytoplasma australiense (PA0090), Prochlorococcus marinus subsp. pastoris str. CCMP1986 (PMM1091), Synechococcus sp. WH 7805 (WH7805_04441), Blattabacterium sp. (Periplaneta americana) str. BPLAN (BPLAN_534), Caldicellulosiruptor obsidiansis OB47 (COB47_0325), Oribacterium sp. oral taxon 078 str. F0262 (GCWU000341_01365), Hydrogenobacter thermophilus TK-6 2 seqs AD046034.1, HTH_1665), Clostridium saccharolyticum WM1 (CLOSA_1248), Prevotella sp. oral taxon 472 str. F0295 (HMPREF6745_1617), Paenibacillus sp. oral taxon 786 str. D14 (POTG_03822), Roseburia inulinivorans DSM 16841 2 seqs ROSEINA2194_02614, ROSEINA2194_02613), Granulicatella elegans ATCC 700633 (HMPREF0446_01381), Prevotella tannerae ATCC 51259 (GCWU000325_02844), Shuttleworthia satelles DSM 14600 (GCWU000342_01722), Phascolarctobacterium succinatutens YIT 12067 (HMPREF9443_01522), Clostridium butyricum E4 str. BoNT E BL5262 (CLP_3980), Caldicellulosiruptor hydrothermalis 108 (CALHY_2287), Caldicellulosiruptor kristjanssonii 177R1B (CALKR_0314), Caldicellulosiruptor owensensis OL (CALOW_0228), Eubacterium cellulosolvens 6 (EUBCEDRAFT_1150), Geobacillus thermoglucosidasius C56-Y593 (GEOTH_0175), Thermincola potens JR (THERJR_0376), Nostoc punctiforme PCC 73102 (NPUN_F5990), Granulicatella adiacens ATCC 49175 (YBBP), Selenomonas flueggei ATCC 43531 (HMPREF0908_1366), Thermocrinis albus DSM 14484 (THAL_0234), Deferribacter desulfuricans SSM1 (DEFDS_1031), Ruminococcus flavefaciens FD-1 (RFLAF_010100012444), Desulfovibrio desulfuricans ND132 (DND132_0877), Clostridium lentocellum DSM 5427 (CLOLE_3370), Desulfovibrio aespoeensis Aspo-2 (DAES_1257), Syntrophothermus lipocalidus DSM 12680 (SLIP_2139), Marivirga tractuosa DSM 4126 (FTRAC_3720), Desulfarculus baarsii DSM 2075 (DEBA_0764), Synechococcus sp. CC9311 (SYNC_1030), Thermaerobacter marianensis DSM 12885 (TMAR_0236), Desulfovibrio sp. FW1012B (DFW101_0480), Jonquetella anthropi E3_33 E1 (GCWU000246_01523), Syntrophobotulus glycolicus DSM 8271 (SGLY_0483), Thermovibrio ammonificans HB-1 (THEAM_0892), Truepera radiovictrix DSM 17093 (TRAD_1704), Bacillus cellulosilyticus DSM 2522 (BCELL_0170), Prevotella veroralis F0319 (HMPREF0973_02947), Erysipelothrix rhusiopathiae str. Fujisawa (ERH_0115), Desulfurispirillum indicum S5 (SELIN_2326), Cyanothece sp. PCC 7424 (PCC7424_0843), Anaerococcus vaginalis ATCC 51170 (YBBP), Aerococcus viridans ATCC 11563 (YBBP), Streptococcus oralis ATCC 35037 2 seqs HMPREF8579_1682, SMSK23_1115), Zunongwangia profunda SM-A87 (ZPR_0978), Halanaerobium hydrogeniformans (HALSA_1882), Bacteroides xylanisolvens XB1A (BXY_29650), Ruminococcus torques L2-14 (RTO_16490), Ruminococcus obeum A2-162 (CK5_33600), Eubacterium rectale DSM 17629 (EUR_24910), Faecalibacterium prausnitzli SL3/3 (FPR_27630), Ruminococcus sp. SR1/5 (CK1_39330), Lachnospiraceae bacterium 3_1_57 FAA_CT1 (HMPREF0994_01490), Lachnospiraceae bacterium 9_1_43 BFAA (HMPREF0987_01591), Lachnospiraceae bacterium 1_4_56 FAA (HMPREF0988_01806), Erysipelotrichaceae bacterium 3_1_53 (HMPREF0983_01328), Ethanoligenens harbinense YUAN-3 (ETHHA_1605), Streptococcus dysgalactiae subsp. dysgalactiae ATCC 27957 (SDD27957_06215), Spirochaeta thermophila DSM 6192 (STHERM_C18370), Bacillus sp. 2_A_57_CT2 (HMPREF1013_05449), Bacillus clausii KSM-K16 (ABCO241), Thermodesulfatator indicus DSM 15286 (THEIN_0076), Bacteroides salanitronis DSM 18170 (BACSA_1486), Oceanithermus profundus DSM 14977 (OCEPR_2178), Prevotella timonensis CRIS 5C-B1 (HMPREF9019_2028), Prevotella buccalis ATCC 35310 (HMPREF0650_0675), Prevotella amnii CRIS 21A-A (HMPREF9018_0365), Bulleidia extructa W1219 (HMPREF9013_0078), Bacteroides coprosuis DSM 18011 (BCOP_0558), Prevotella multisaccharivorax DSM 17128 (PREMU_0839), Cellulophaga algicola DSM 14237 (CELAL_0483), Synechococcus sp. WH 5701 (WH5701_10360), Desulfovibrio africanus str. Walvis Bay (DESAF_3283), Oscillibacter valericigenes Sjm18-20 (OBV_23340), Deinococcus proteolyticus MRP (DEIPR_0134), Bacteroides helcogenes P 36-108 (BACHE_0366), Paludibacter propionicigenes WB4 (PALPR_1923), Desulfotomaculum nigrificans DSM 574 (DESNIDRAFT_2093), Arthrospira platensis NIES-39 (BAI89442.1), Mahella australiensis 50-1 BON (MAHAU_1846), Thermoanaerobacter wiegelii Rt8.B1 (THEWI_2191), Ruminococcus albus 7 (RUMAL_2345), Staphylococcus lugdunensis HKU09-01 (SLGD_00862), Megasphaera genomosp. type_1 str. 28L (HMPREF0889_1099), Clostridiales genomosp. BVAB3 str. UPII9-5 (HMPREF0868_1453), Pediococcus claussenii ATCC BAA-344 (PECL_571), Prevotella oulorum F0390 (HMPREF9431_01673), Turicibacter sanguinis PC909 (CUW_0305), Listeria seeligeri FSL N1-067 (NT03LS_2473), Solobacterium moorei F0204 (HMPREF9430_01245), Megasphaera micronuciformis F0359 (HMPREF9429_00929), Capnocytophaga sp. oral taxon 329 str. F0087 2 seqs HMPREF9074_00867, HMPREF9074_01078), Streptococcus anginosus F0211 (HMPREF0813_00157), Mycoplasma suis KI3806 (MSUI04040), Mycoplasma gallisepticum str. F (MGF_2771), Deinococcus maricopensis DSM 21211 (DEIMA_0651), Odoribacter splanchnicus DSM 20712 (ODOSP_0239), Lactobacillus fermentum CECT 5716 (LC40_0265), Lactobacillus iners AB-1 (LINEA_010100006089), cyanobacterium UCYN-A (UCYN_03150), Lactobacillus sanfranciscensis TMW 1.1304 (YBBP), Mucilaginibacter paludis DSM 18603 (MUCPA_1296), Lysinibacillus fusiformis ZC1 (BFZC1_03142), Paenibacillus vortex V453 (PVOR_30878), Waddlia chondrophila WSU 86-1044 (YBBP), Flexistipes sinusarabici DSM 4947 (FLEXSI_0971), Paenibacillus curdlanolyticus YK9 (PAECUDRAFT_1888), Clostridium cf. saccharolyticum K10 (CLS_03290), Alistipes shahii WAL 8301 (AL1_02190), Eubacterium cylindroides T2-87 (EC1_00230), Coprococcus catus GD/7 (CC1_32460), Faecalibacterium prausnitzii L2-6 (FP2_09960), Clostridium clariflavum DSM 19732 (CLOCL_2983), Bacillus atrophaeus 1942 (BATR1942_19530), Mycoplasma pneumoniae FH (MPNE_0277), Lachnospiraceae bacterium 2_1_46 FAA (HMPREF9477_00058), Clostridium symbiosum WAL-14163 (HMPREF9474_01267), Dysgonomonas gadei ATCC BAA-286 (HMPREF9455_02764), Dysgonomonas mossii DSM 22836 (HMPREF9456_00401), Thermus scotoductus SA-01 (TSC_C24350), Sphingobacterium sp. 21 (SPH21_1233), Spirochaeta caldaria DSM 7334 (SPICA_1201), Prochlorococcus marinus str. MIT 9312 (PMT9312_1102), Prochlorococcus marinus str. MIT 9313 (PMT_1058), Faecalibacterium cf. prausnitzii KLE1255 (HMPREF9436_00949), Lactobacillus crispatus ST1 (LCRIS_00721), Clostridium ljungdahlii DSM 13528 (CLJU_C40470), Prevotella bryantii B14 (PBR_2345), Treponema phagedenis F0421 (HMPREF9554_02012), Clostridium sp. BNL1100 (CLO1100_2851), Microcoleus vaginatus FGP-2 (MICVADRAFT_1377), Brachyspira pilosicoli 95/1000 (BP951000_0671), Spirochaeta coccoides DSM 17374 (SPICO_1456), Haliscomenobacter hydrossis DSM 1100 (HALHY_5703), Desulfotomaculum kuznetsovii DSM 6115 (DESKU_2883), Runella slithyformis DSM 19594 (RUNSL_2859), Leuconostoc kimchii IMSNU 11154 (LKI_08080), Leuconostoc gasicomitatum LMG 18811 (OSSG), Pedobacter saltans DSM 12145 (PEDSA_3681), Paraprevotella xylaniphila YIT 11841 (HMPREF9442_00863), Bacteroides clarus YIT 12056 (HMPREF9445_01691), Bacteroides fluxus YIT 12057 (HMPREF9446_03303), Streptococcus urinalis 2285-97 (STRUR_1376), Streptococcus macacae NCTC 11558 (STRMA_0866), Streptococcus ictaluri 707-05 (STRIC_0998), Oscillochloris trichoides DG-6 (OSCT_2821), Parachlamydia acanthamoebae UV-7 (YBBP), Prevotella denticola F0289 (HMPREF9137_0316), Parvimonas sp. oral taxon 110 str. F0139 (HMPREF9126_0534), Calditerrivibrio nitroreducens DSM 19672 (CALNI_1443), Desulfosporosinus orientis DSM 765 (DESOR_0366), Streptococcus mitis by. 2 str. F0392 (HMPREF9178_0602), Thermodesulfobacterium sp. OPB45 (TOPB45_1366), Synechococcus sp. WH 8102 (SYNW0935), Thermoanaerobacterium xylanolyticum LX-11 (THEXY_0384), Mycoplasma haemofelis Ohio2 (MHF_1192), Capnocytophaga canimorsus Cc5 (CCAN_16670), Pediococcus acidilactici DSM 20284 (HMPREF0623_1647), Prevotella marshii DSM 16973 (HMPREF0658_1600), Peptoniphilus duerdenii ATCC BAA-1640 (HMPREF9225_1495), Bacteriovorax marinus SJ (BMS_2126), Selenomonas sp. oral taxon 149 str. 67H29BP (HMPREF9166_2117), Eubacterium yurii subsp. margaretiae ATCC 43715 (HMPREF0379_1170), Streptococcus mitis ATCC 6249 (HMPREF8571_1414), Streptococcus sp. oral taxon 071 str. 73H25AP (HMPREF9189_0416), Prevotella disiens FB035-09AN (HMPREF9296_1148), Aerococcus urinae ACS-120-V-Col10a (HMPREF9243_0061), Veillonella atypica ACS-049-V-Sch6 (HMPREF9321_0282), Cellulophaga lytica DSM 7489 (CELLY_2319), Thermaerobacter subterraneus DSM 13965 (THESUDRAFT_0411), Desulfurobacterium thermolithotrophum DSM 11699 (DESTER_0391), Treponema succinifaciens DSM 2489 (TRESU_1152), Marinithermus hydrothermalis DSM 14884 (MARKY_1861), Streptococcus infantis SK1302 (SIN_0824), Streptococcus parauberis NCFD 2020 (SPB_0808), Streptococcus porcinus str. Jelinkova 176 (STRPO_0164), Streptococcus criceti HS-6 (STRCR_1133), Capnocytophaga ochracea F0287 (HMPREF1977_0786), Prevotella oralis ATCC 33269 (HMPREF0663_10671), Porphyromonas asaccharolytica DSM 20707 (PORAS_0634), Anaerococcus prevotii ACS-065-V-Col13 (HMPREF9290_0962), Peptoniphilus sp. oral taxon 375 str. F0436 (HMPREF9130_1619), Veillonella sp. oral taxon 158 str. F0412 (HMPREF9199_0189), Selenomonas sp. oral taxon 137 str. F0430 (HMPREF9162_2458), Cyclobacterium marinum DSM 745 (CYCMA_2525), Desulfobacca acetoxidans DSM 11109 (DESAC_1475), Listeria ivanovii subsp. ivanovii PAM 55 (LIV_2111), Desulfovibrio vulgaris str. Hildenborough (DVU_1280), Desulfovibrio vulgaris str. ‘Miyazaki F’ (DVMF_0057), Muricauda ruestringensis DSM 13258 (MURRU_0474), Leuconostoc argentinum KCTC 3773 (LARGK3_010100008306), Paenibacillus polymyxa SC2 (PPSC2_C4728), Eubacterium saburreum DSM 3986 (HMPREF0381_2518), Pseudoramibacter alactolyticus ATCC 23263 (HMP0721_0313), Streptococcus parasanguinis ATCC 903 (HMPREF8577_0233), Streptococcus sanguinis ATCC 49296 (HMPREF8578_1820), Capnocytophaga sp. oral taxon 338 str. F0234 (HMPREF9071_1325), Centipeda periodontii DSM 2778 (HMPREF9081_2332), Prevotella multiformis DSM 16608 (HMPREF9141_0346), Streptococcus peroris ATCC 700780 (HMPREF9180_0434), Prevotella salivae DSM 15606 (HMPREF9420_1402), Streptococcus australis ATCC 700641 2 seqs HMPREF9961_0906, HMPREF9421_1720), Streptococcus cristatus ATCC 51100 2 seqs HMPREF9422_0776, HMPREF9960_0531), Lactobacillus acidophilus 30SC (LAC30SC_03585), Eubacterium limosum KIST612 (ELI_0726), Streptococcus downei F0415 (HMPREF9176_1204), Streptococcus sp. oral taxon 056 str. F0418 (HMPREF9182_0330), Oribacterium sp. oral taxon 108 str. F0425 (HMPREF9124_1289), Streptococcus vestibularis F0396 (HMPREF9192_1521), Treponema brennaborense DSM 12168 (TREBR_1165), Leuconostoc fallax KCTC 3537 (LFALK3_010100008689), Eremococcus coleocola ACS-139-V-Col8 (HMPREF9257_0233), Peptoniphilus harei ACS-146-V-Sch2b (HMPREF9286_0042), Clostridium sp. HGF2 (HMPREF9406_3692), Alistipes sp. HGBS (HMPREF9720_2785), Prevotella dentalis DSM 3688 (PREDE_0132), Streptococcus pseudoporcinus SPIN 20026 (HMPREF9320_0643), Dialister microaerophilus UPII 345-E (HMPREF9220_0018), Weissella cibaria KACC 11862 (WCIBK1_010100001174), Lactobacillus coryniformis subsp. coryniformis KCTC 3167 (LCORCK3_010100001982), Synechococcus sp. PCC 7335 (S7335_3864), Owenweeksia hongkongensis DSM 17368 (OWEHO_3344), Anaerolinea thermophila UNI-1 (ANT_09470), Streptococcus oralis Uo5 (SOR_0619), Leuconostoc gelidum KCTC 3527 (LGELK3_010100006746), Clostridium botulinum BKT015925 (CBC4_0275), Prochlorococcus marinus str. MIT 9211 (P9211_10951), Prochlorococcus marinus str. MIT 9215 (P9215_12271), Staphylococcus aureus subsp. aureus NCTC 8325 (SAOUHSC_02407), Staphylococcus aureus subsp. aureus COL (SACOL2153), Lactobacillus animalis KCTC 3501 (LANIK3_010100000290), Fructobacillus fructosus KCTC 3544 (FFRUK3_010100006750), Acetobacterium woodii DSM 1030 (AWO_C28200), Planococcus donghaensis MPA1U2 (GPDM_12177), Lactobacillus farciminis KCTC 3681 (LFARK3_010100009915), Melissococcus plutonius ATCC 35311 (MPTP_0835), Lactobacillus fructivorans KCTC 3543 (LFRUK3_010100002657), Paenibacillus sp. HGF7 (HMPREF9413_5563), Lactobacillus oris F0423 (HMPREF9102_1081), Veillonella sp. oral taxon 780 str. F0422 (HMPREF9200_1112), Parvimonas sp. oral taxon 393 str. F0440 (HMPREF9127_1171), Tetragenococcus halophilus NBRC 12172 (TEH_13100), Candidatus Chloracidobacterium thermophilum B (CABTHER_A1277), Ornithinibacillus scapharcae TW25 (OTW25_010100020393), Lacinutrix sp. 5H-3-7-4 (LACAL_0337), Krokinobacter sp. 4H-3-7-5 (KRODI_0177), Staphylococcus pseudintermedius ED99 (SPSE_0659), Staphylococcus aureus subsp. aureus MSHR1132 (CCE59824.1), Paenibacillus terrae HPL-003 (HPL003_03660), Caldalkalibacillus thermarum TA2.A1 (CATHTA2_0882), Desmospora sp. 8437 (HMPREF9374_2897), Prevotella nigrescens ATCC 33563 (HMPREF9419_1415), Prevotella pallens ATCC 700821 (HMPREF9144_0175), Streptococcus infantis X (HMPREF1124.

In some embodiments, the genetically engineered bacteria are capable of increasing c-di-AMP levels in the tumor microenvironment. In some embodiments, the genetically engineered bacteria are capable of increasing c-diAMP levels in the intracellular space in a tumor. In some embodiments, the genetically engineered bacteria are capable of increasing c-diAMP levels inside of a eukaryotic cell. In some embodiments, the genetically engineered bacteria are capable of increasing c-diAMP levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c-GAMP (2′3′ or 3′3′) and/or cyclic-di-GMP levels inside of a cancer cell. In some embodiments, the genetically engineered bacteria are capable of increasing c-di-AMP levels in vitro in the bacterial cell and/or in the growth medium.

In any of these embodiments, the bacteria genetically engineered to produce cyclic-di-AMP produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more cyclic-di-AMP than unmodified bacteria of the same bacterial subtype under the same conditions. In yet another embodiment, the genetically engineered bacteria produce at least about 0 to 1.0-fold, 1.0-1.2-fold, 1.2-1.4-fold, 1.4-1.6-fold, 1.6-1.8-fold, 1.8-2-fold, or two-fold more cyclic-di-AMP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-AMP than unmodified bacteria of the same bacterial subtype under the same conditions.

In any of these embodiments, the bacteria genetically engineered to produce cyclic-di-AMP consume at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume at least about 0 to 1.0-fold, 1.0-1.2-fold, 1.2-1.4-fold, 1.4-1.6-fold, 1.6-1.8-fold, 1.8-2-fold, or two-fold more ATP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-AMP than unmodified bacteria of the same bacterial subtype under the same conditions.

In any of these embodiments, the bacteria genetically engineered to produce cyclic-di-GAMP produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions. In yet another embodiment, the genetically engineered bacteria produce at least about 0 to 1.0-fold, 1.0-1.2-fold, 1.2-1.4-fold, 1.4-1.6-fold, 1.6-1.8-fold, 1.8-2-fold, or two-fold more cyclic-di-GAMP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-GAMP than unmodified bacteria of the same bacterial subtype under the same conditions.

In any of these embodiments, the bacteria genetically engineered to produce cyclic-di-GAMP consume at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume at least about 0 to 1.0-fold, 1.0-1.2-fold, 1.2-1.4-fold, 1.4-1.6-fold, 1.6-1.8-fold, 1.8-2-fold, or two-fold more ATP and/or GTP than unmodified bacteria of the same bacterial subtype under the same conditions. In yet another embodiment, the genetically engineered bacteria consume at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more ATP and/or GTP than unmodified bacteria of the same bacterial subtype under the same conditions.

In any of these embodiments, the genetically engineered bacteria increase STING agonist production rate by at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the STING agonist production rate by at least about 0 to 1.0-fold, 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase STING agonist production rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one embodiment, the genetically engineered bacteria increase STING agonist production by at least about 80% to 100% relative to unmodified bacteria of the same bacterial subtype under the same conditions, after 4 hours. In one embodiment, the genetically engineered bacteria increase STING agonist production by at least about 90% to 100% relative to unmodified bacteria of the same bacterial subtype under the same conditions after 4 hours. In one specific embodiment, the genetically engineered bacteria increase STING agonist production by at least about 95% to 100% relative to unmodified bacteria of the same bacterial subtype under the same conditions, after 4 hours. In one specific embodiment, the genetically engineered bacteria increase the STING agonist production by at least about 99% to 100% relative to unmodified bacteria of the same bacterial subtype under the same conditions, after 4 hours. In yet another embodiment, the genetically engineered bacteria increase the STING agonist production by at least about 10-50 fold after 4 hours. In yet another embodiment, the genetically engineered bacteria increase STING agonist production by at least about 50-100 fold after 4 hours. In yet another embodiment, the genetically engineered bacteria increase STING agonist production by at least about 100-500 fold after 4 hours. In yet another embodiment, the genetically engineered bacteria increase STING agonist production by at least about 500-1000 fold after 4 hours. In yet another embodiment, the genetically engineered bacteria increase the STING agonist production by at least about 1000-5000 fold after 4 hours. In yet another embodiment, the genetically engineered bacteria increase the STING agonist production by at least about 5000-10000 fold after 4 hours. In yet another embodiment, the genetically engineered bacteria increase STING agonist production by at least about 10000-1000 fold after 4 hours.

In any of these STING agonist production embodiments, the genetically engineered bacteria are capable of reducing tumor cell proliferation (in vitro during cell culture and/or in vivo) by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production embodiments, the genetically engineered bacteria are capable of reducing tumor growth by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production embodiments, the genetically engineered bacteria are capable of reducing tumor size by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these agonist STING production embodiments, the genetically engineered bacteria are capable of reducing tumor volume by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production embodiments, the genetically engineered bacteria are capable of reducing tumor weight by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria comprising gene sequences encoding dacA (and/or another enzyme for the production of a STING agonists, e.g., cGAS) are able to increase IFN-β1 mRNA or protein levels in macrophages and/or dendritic cells, e.g., in cell culture. In some embodiments, the IFN-β1 mRNA or protein increase dependent on the dose of bacteria administered. In some embodiments, the genetically engineered bacteria comprising gene sequences encoding dacA (and/or another enzyme for the production of a STING agonists, e.g., cGAS) are able to increase IFN-β1 mRNA or protein levels in macrophages and/or dendritic cells, e.g., in the tumor. In some embodiments, the IFN-beta1 mRNA or protein increase is dependent on the dosage of bacteria administered.

In one embodiment, IFN-beta1 mRNA or protein production in tumors is about two-fold, about 3-fold, about 4-fold as compared to levels of IFN-beta1 production observed upon administration of an unmodified bacteria of the same subtype under the same conditions, e.g., at day 2 after first injection of the bacteria. In some embodiments, the genetically engineered bacteria induce the production of at least about 6,000 to 25,000, 15,000 to 25,000, 6,000 to 8,000, 20,000 to 25,000 pg/ml IFN b1 mRNA in bone marrow-derived dendritic cells, e.g., at 4 hours post-stimulation.

In some embodiments, the genetically engineered bacteria comprising gene sequences encoding dacA (or another enzyme for the production of a STING agonists) can dose-dependently increase IFN-b1 production in bone marrow-derived dendritic cells, e.g., at 2 or 4 hours post stimulation.

In some embodiments, the genetically engineered bacteria comprising gene sequences encoding dacA (or another enzyme for the production of a STING agonists) are able to reduce tumor volume, e.g., at 4 or 9 days after a regimen of 3 bacterial treatments, relative to an unmodified bacteria of the same subtype under the same conditions. In a non-limiting example, the tumor volume is about 0 to 30 mm3 after 9 days.

In some embodiments, the tumor volume at day 1, 4, and 12 or three times a week for 27 days or longer. In some embodiments, complete tumor rejection is observed.

Tumor volume in models in mice can be used to characterize strain activity. For example, the tumor volume may be measured at day 1, 4, and 12 or three times a week for 27 days or longer in a tumor model such as the A20 B cell lymphoma model, or other models described herein or known in the art. Different doses may be administered to establish show a dose dependent response and to establish efficacy and tolerability. Tumor volume may be compared between an animal administered the STING agonist strain and the strain without the STING circuitry of the same subtype under the same conditions. In some embodiments, the tumor volume may be measured at day 1, 4, and 12 or three times a week for 27 days or longer. In one embodiment, tumor volume is at least about 1 to 2-fold, 2 to 3-fold, 3 to 4-fold, 4 to 5-fold, 5 to 6-fold, 6 to 7-fold or 7 to 8-fold reduced in the STING producing strain as compared to the unmodified strains of the same subtype under the same conditions, e.g., as assessed in the A20 model. In one embodiment, tumor volume can be compared in the A20 mouse model between the STING producing strain and the unmodified strain of the same subtype under the same conditions at 5, 8 or 12 days. In one embodiment, tumor volume is at least about 6-fold reduced at 12 days upon administration with the STING producing strain at 10\hat{ }8 CFU as compared to the unmodified strains of the same subtype under the same conditions after 12 days. In one embodiment, tumor volume is at least about 2-fold to 3-fold reduced at 12 days upon administration with the STING producing strain at 10\hat{ }7 CFU as compared to the unmodified strains of the same subtype under the same conditions after 12 days. In one embodiment, tumor volume is at least about 3-fold to 4-fold reduced at 12 days upon administration with the STING producing strain at 10\hat{ }7 CFU as compared to the unmodified strains of the same subtype under the same conditions after 12 days.

Strain activity of the STING agonist producing strain can be defined by conducting in vitro measurements c-di-AMP production (in the cell or in the medium). C-di-AMP production can be measured over a time period of 1, 2, 3, 4, 5, 6 hours or greater. In one example, c-di-AMP levels can be measured at 0, 2, or 4 hours. Unmodified Nissle can be used as a baseline in such measurements. If STING agonist producing enzyme is under the control of a promoter which is induced by a chemical inducer, the inducer needs to be added. If STING agonist producing enzyme is under the control of a promoter which is induced by exogenous environmental conditions, such as low-oxygen conditions, the bacterial cells are induced under these conditions, e.g., low oxygen conditions. As an additional baseline measurement, STING agonist producing strains which are inducible can be left uninduced. After the incubation time, levels of c-diAMP can be measured by LC-MS as described herein. In some embodiments, the induced STING agonist producing strain is capable of producing c-di-AMP at a concentration of at least about 0.01 mM to 1.4 mM per 10\hat{ }9. In some embodiments, the induced STING agonist producing strain is capable of producing c-di-AMP at a concentration of at least about 0.01 mM to 0.02 mM, 0.02 mM to 0.03 mM, 0.03 mM to 0.04 mM, 0.04 mM to 0.05 mM, 0.05 mM to 0.06 mM, 0.06 mM to 0.07 mM, 0.07 mM to 0.08 mM, 0.08 mM to 0.09 mM, 0.09 mM to 0.10 mM, 0.10 mM to 0.12 mM per 10\hat{ }9 e.g., after 2 or 4 hours. In some embodiments, the induced STING agonist producing strain is capable of producing c-di-AMP at a concentration of at least about 0.1 mM to 0.2 mM, 0.2 mM to 0.3 mM, 0.3 mM to 0.4 mM, 0.4 mM to 0.5 mM, 0.5 mM to 0.6 mM, 0.6 mM to 0.7 mM, 0.7 mM to 0.8 mM, 0.8 mM to 0.9 mM, 0.9 mM to 1 mM, 1 mM to 1.2 mM, 1.2 mM to 1.3 mM, 1.3 mM to 1.4 mM per 10\hat{ }9 e.g., after 2 or 4 hours.

Strain activity of the STING agonist producing strain may also be measured using in vitro measurements of activity. In a non-limiting example of an in vitro strain activity measurement, IFN-beta1 induction in RAW 264.7 cells (or other macrophage or dendritic cell) in culture may be measured. Activity of the strain can be measured at various multiplicities of infection (MOI) at various time points. For example, activity can be measured at 1, 2, 3, 4, 5, 6 hours or greater. In one example activity can be measured at 45 minutes or 4 hours. Unmodified Nissle can be used as a baseline in such measurements. If STING agonist producing enzyme is under the control of a promoter which is induced by a chemical inducer, the inducer needs to be added. If STING agonist producing enzyme is under the control of a promoter which is induced by exogenous environmental conditions, such as low-oxygen conditions, the bacterial cells are induced under these conditions, e.g., low oxygen conditions. As an additional baseline measurement, STING agonist producing strains which are inducible can be left uninduced. After the incubation time, IFN-beta levels can be measured from protein extracts or RNA levels can be analyzed, e.g., via PCT based methods. In some embodiments, the induced STING agonist producing strain can elicit a dose-dependent induction of IFN-b levels. In some embodiments, 10\hat{ }1 to 10\hat{ }2 (multiplicities of infection (MOI) can induce at least about 20 to 25 times, 25 to 30 times, 30 to 35 times, 35 to 40 times or more greater IFN-beta levels as the unmodified Nissle baseline strain of the same subtype under the same conditions, eg., after 4 hours. In some embodiments, 10\hat{ }1 to 10\hat{ }2 (multiplicities of infection (MOI) can induce at least about 10,000 to 12,000, 12,000 to 15,000, 15,000 to 20,000 or 20,000 to 25,000 pg/ml media IFN-beta e.g., after 4 hours.

In some embodiments, 10\hat{ }1 to 10\hat{ }2 (multiplicities of infection (MOI) can induce at least about 10 to 12 times, 12 to 15 times, 15 to 20 times, 20 to 25 times or more greater IFN-beta levels as the wild type Nissle baseline strain of the same subtype under the same conditions, e.g., after 45 minutes. In some embodiments, 10\hat{ }1 to 10\hat{ }2 (multiplicities of infection (MOI) can induce at least about 4,000 to 6,000, 6,000 to 8,000, 8,000 to 10,000 or 10,000 to 12,000 pg/ml media IFN-beta e.g., after 45 minutes.

In some embodiments, the bacteria genetically engineered to produce STING agonists are capable of increasing the response rate by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the genetically engineered bacteria comprising gene sequences encoding dacA, achieve a 100% response rate.

In some embodiments, the response rate is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the response rate is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides, achieve a tumor regression by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the tumor regression is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the tumor regression is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides increase total T cell numbers in the tumor draining lymph nodes. In some embodiments, the increase in total T cell numbers in the tumor draining lymph nodes is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the increase in total T cell numbers is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the increase in total T cell numbers is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides increase the percentage of activated effector CD4 and CD8 T cells in tumor draining lymph nodes.

In some embodiments, the percentage of activated effector CD4 and CD8 T cells in the tumor draining lymph nodes is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the percentage of activated effector CD4 and CD8 T cells is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the percentage of activated effector CD4 and CD8 T cells is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold more than observed with unmodified bacteria of the same bacterial subtype under the same conditions. In one embodiment, the gene encoded by the bacteria is DacA and the percentage of activated effector CD4 and CD8 T cells is two to four fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides achieve early rise of innate cytokines inside the tumor and a later rise of an effector-T-cell response.

In some embodiments, the genetically engineered bacteria comprising gene sequences encoding dacA (or other enzymes for production of STING agonists) in the tumor microenvironment are able to overcome immunological suppression and generating robust innate and adaptive antitumor immune responses. In some embodiments, the genetically engineered bacteria comprising gene sequences encoding dacA inhibit proliferation or accumulation of regulatory T cells.

In some embodiments, the genetically engineered bacteria comprising gene sequences encoding dacA, cGAS, and/or other enzymes for production of STING agonists, achieve early rise of innate cytokines inside the tumor, including but not limited to IL-6, IL-1beta, and MCP-1.

In some embodiments IL-6 is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more induced as compared to an unmodified bacteria of the same subtype under the same conditions. In some embodiments, IL-6 is at least about 0 to 1.0-fold, 1.0-1.2-fold, 1.2-1.4-fold, 1.4-1.6-fold, 1.6-1.8-fold, 1.8-2-fold, or two-fold more induced than observed with than unmodified bacteria of the same bacterial subtype under the same conditions. In yet another embodiment, the IL-6 is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more induced than observed with unmodified bacteria of the same bacterial subtype under the same conditions. In one embodiment, the gene encoded by the bacteria is dacA and the levels of induced IL-6 is about two to three-fold greater than observed with unmodified bacteria of the same bacterial subtype under the same conditions.

In some embodiments, the levels of IL-1beta in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-1beta are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-1beta are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of IL-1beta are about 2 fold, 3 fold, or 4 fold more than observed with unmodified bacteria of the same bacterial subtype under the same conditions.

In some embodiments, the levels of MCP1 in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of MCP1 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of MCP1 are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of MCP1 are about 2-fold, 3-fold, or 4-fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides achieve activation of molecules relevant towards an effector-T-cell response, including but not limited to, Granzyme B, IL-2, and IL-15.

In some embodiments, the levels of granzyme B in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of granzyme B are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of granzyme B are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of granzyme B are about 2 fold, 3 fold, or 4 fold more than observed with unmodified bacteria of the same bacterial subtype under the same conditions.

In some embodiments, the levels of IL-2 in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-2 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-2 are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is DacA and the levels of IL-2 are about 3 fold, 4 fold, or 5 fold more than observed with unmodified bacteria of the same bacterial subtype under the same conditions.

In some embodiments, the levels of IL-15 in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-15 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-15 are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, gene encoded by the bacteria is DacA and the levels of IL-15 are about 2-fold, 3-fold, -fold, or 5-fold more than observed with unmodified bacteria of the same bacterial subtype under the same conditions.

In some embodiments, the levels of IFNg in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IFNg are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IFNg are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, di-GAMP synthase, and/or other STING agonist producing polypeptide and levels of IFNg are about 2 fold, 3 fold, or 4 fold more than observed with unmodified bacteria of the same bacterial subtype under the same conditions.

In some embodiments, the levels of IL-12 in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-12 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-12 are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of IL-12 are about 2 fold, 3 fold, or 4 fold more than observed with unmodified bacteria of the same bacterial subtype under the same conditions.

In some embodiments, the levels of TNF-a in the tumor is at least about 0% to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of TNF-a are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of TNF-a are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of TNF-a are at least about 2 fold, 3 fold, or 4 fold more than observed with unmodified bacteria of the same bacterial subtype under the same conditions.

In some embodiments, the levels of GM-CSF in the tumor is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of GM-CSF are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of GM-CSF are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of GM-CSF are at least about 2 fold, 3 fold, or 4 fold more than observed with unmodified bacteria of the same bacterial subtype under the same conditions.

In some embodiments, administration of the genetically engineered bacteria comprising gene sequences encoding one or more of a diadenylate cyclase, e.g., DacA, a di-GAMP synthase, and/or other STING agonist producing polypeptide results in long-term immunological memory. In some embodiments, long term immunological memory is established, exemplified by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more protection from secondary tumor challenge compared to naïve age-matched controls. In some embodiments, long term immunological memory is established, exemplified by at least about 0 to 1.0-fold, 1.0-1.2-fold, 1.2-1.4-fold, 1.4-1.6-fold, 1.6-1.8-fold, 1.8-2-fold, or two-fold or more protection from secondary tumor challenge compared to naïve age-matched controls. In yet another embodiment, long term immunological memory is established, exemplified by at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more protection from secondary tumor challenge compared to naïve age-matched controls.

In some embodiments, the c-di-GAMP synthases, diadenylate cyclases, or other STING agonist producing polypeptides are modified and/or mutated, e.g., to enhance stability, or to increase STING agonism. In some embodiments, c-di-GAMP synthases from Vibrio cholerae or the orthologs thereof thereof (e.g., from Verminephrobacter eiseniae, Kingella denitrificans, and/or Neisseria bacilliformis) or human cGAS is modified and/or mutated, e.g., to enhance stability, or to increase STING agonism. In some embodiments, the diadenylate cyclase from Listeria monocytogenes is modified and/or mutated, e.g., to enhance stability, or to increase STING agonism.

In some embodiments, the genetically engineered bacteria and/or other microorganisms are capable of producing one or more diadenylate cyclases, c-di-GAMP synthases and/or other STING agonist producing polypeptides under inducing conditions, e.g., under a condition(s) associated with immune suppression and/or tumor microenvironment. In some embodiments, the genetically engineered bacteria and/or other microorganisms are capable of producing the diadenylate cyclases, c-di-GAMP synthases and/or other STING agonist producing polypeptides in low-oxygen conditions or hypoxic conditions, in the presence of certain molecules or metabolites, in the presence of molecules or metabolites associated with cancer, or certain tissues, immune suppression, or inflammation, or in the presence of a metabolite that may or may not be present in the gut, circulation, or the tumor, and which may be present in vitro during strain culture, expansion, production and/or manufacture such as arabinose, cumate, and salicylate. In some embodiments, the one or more genetically engineered bacteria comprise gene sequence(s) encoding the diadenylate cyclases, c-di-GAMP synthases and/or other STING agonist producing polypeptides, wherein the diadenylate cyclases, c-di-GAMP synthases and/or other STING agonist producing polypeptides are operably linked to a promoter inducible by exogenous environmental conditions of the tumor microenvironment. In some embodiments, the exogenous environmental conditions of the tumor microenvironment are low oxygen conditions. In some embodiments, the one or more genetically engineered bacteria comprise gene sequence(s) encoding the diadenylate cyclases, c-di-GAMP synthases and/or other STING agonist producing polypeptides, wherein the diadenylate cyclases, c-di-GAMP synthases and/or other STING agonist producing polypeptides is operably linked to a promoter inducible by cumate or salicylate as described herein. In some embodiments, the gene sequences encoding diadenylate cyclases, c-di-GAMP synthases and/or other STING agonist producing polypeptides are operably linked to a constitutive promoter. In some embodiments, the gene sequences encoding diadenylate cyclases, c-di-GAMP synthases and/or other STING agonist producing polypeptides are present on one or more plasmids (e.g., high copy or low copy) or are integrated into one or more sites in the bacteria and/or other microorganism chromosome(s).

In any of these embodiments, any of the STING agonist producing strains described herein may comprise an auxotrophic modification. In any of these embodiments, the STING agonist producing strains may comprise an auxotrophic modification in DapA, e.g., a deletion or mutation in DapA. In any of these embodiments, the STING agonist producing strains may further comprise an auxotrophic modification in ThyA e.g., a deletion or mutation in ThyA. In any of these embodiments, the STING agonist producing strains may comprise a DapA and a ThyA auxotrophy. In any of these embodiments, the bacteria may further comprise an endogenous phage modification, e.g., a mutation or deletion, in an endogenous phage. In a non-limiting example the bacterial host is E. coli Nissle and the phage modification comprises a modification in Nissle Phage 3, described herein. In one example, the phage modification is a deletion of one or more genes, e.g., a 10 kb deletion.

In any of these embodiments describing genetically engineered bacteria comprising gene sequences encoding one or more diadenylate cyclases, c-di-GAMP synthases or other STING agonist producing polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases, c-di-GAMP synthases or other STING agonist producing polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding diadenylate cyclase e.g., DacA, e.g., from Listeria monocytogenes, wherein diadenylate cyclase gene is operably linked to a promoter inducible under exogenous environmental conditions, e.g., conditions in the tumor microenvironment. In one embodiment, the diadenylate cyclase gene is operably linked to a promoter inducible under low oxygen conditions, e.g., a FNR promoter. In certain embodiments, one or more genetically engineered bacteria comprise gene sequence(s) encoding diadenylate cyclase, e.g., dacA, e.g., from Listeria monocytogenes, wherein diadenylate cyclase is operably linked to a promoter inducible by cumate or salicylate as described herein. In certain embodiments, the diadenylate cyclase gene sequences are integrated into the bacterial chromosome. Suitable integration sites are described herein. In a non-limiting example the diadenylate cyclase gene is integrated at HA910. In certain embodiments, the bacteria comprising gene sequences encoding the diadenylate cyclase further comprise an auxotrophic modification. In some embodiments, the modification, e.g., a mutation or deletion is in the dapA gene. In some embodiments, the modification, e.g., a mutation or deletion is in the thyA gene. In some embodiments, the modification, e.g., a mutation or deletion is in both dapA and thyA genes. In any of these embodiments, the bacteria may further comprise a phage modification, e.g., a mutation or deletion in an endogenous prophage. In one example, the prophage modification is a deletion of one or more genes, e.g., a 10 kb deletion. In a non-limiting example, the genetically engineered bacteria comprising gene sequences encoding diadenylate cyclase are derived from E. coli Nissle and the prophage modification comprises a deletion or mutation in Nissle Prophage 3, described herein.

In certain embodiments genetically engineered bacteria comprising gene sequences encoding one or more diadenylate cyclases, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one specific embodiment, one or more genetically engineered bacteria comprise gene sequence(s) encoding diadenylate cyclase e.g., DacA, e.g., from Listeria monocytogenes, wherein the diadenylate cyclase gene is operably linked to a promoter inducible under low oxygen conditions, e.g., a FNR promoter. The dacA gene sequences are integrated into the bacterial chromosome, e.g., at integration site HA910. The bacteria further comprise a auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding cGAMP synthase e.g., human cGAS, wherein the cGAS gene is operably linked to a promoter inducible under exogenous environmental conditions, e.g., conditions in the tumor microenvironment. In one embodiment, the cGAS gene is operably linked to a promoter inducible under low oxygen conditions, e.g., a FNR promoter. In certain embodiments, one or more genetically engineered bacteria comprise gene sequence(s) encoding cGAS, e.g., human cGAS, wherein the cGAS gene is operably linked to a promoter inducible by cumate or salicylate as described herein. In certain embodiments, the cGAS gene sequences are integrated into the bacterial chromosome. Suitable integration sites are described herein and known in the art. In certain embodiments, the bacteria comprising gene sequences encoding cGAS further comprise an auxotrophic modification, e.g., a mutation or deletion in dapA or thyA or both genes. In some embodiments, the modification, e.g., a mutation or deletion is in the dapA gene. In some embodiments, the modification, e.g., a mutation or deletion is in thyA gene. In some embodiments, the modification, e.g., a mutation or deletion is in both dapA and thyA genes. In any of these embodiments, the bacteria may further comprise a prophage modification, e.g., a mutation or deletion, in an endogenous prophage. In one example, the prophage modification is a deletion of one or more genes, e.g., a 10 kb deletion. In a non-limiting example, the genetically engineered bacteria comprising gene sequences encoding cGAS are derived from E. coli Nissle and the prophage modification comprises a deletion or mutation in Nissle Phage 3, described herein.

In any of these embodiments describing genetically engineered bacteria comprising gene sequences encoding one or more cGAS, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more cGAS may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one embodiment, one or more genetically engineered bacteria comprise gene sequence(s) encoding cGAS e.g., human cGAS, wherein the cGAS gene is operably linked to a promoter inducible under low oxygen conditions, e.g., an FNR promoter. The cGAS gene sequences are integrated into the bacterial chromosome. The bacteria further comprise an auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria comprising gene sequences encoding one or more cGAS, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more cGAS may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding diadenylate cyclase e.g., DacA, e.g., from Listeria monocytogenes, and cGAMP synthase e.g., human cGAS. In certain embodiments, the diadenylate cyclase gene and/or the cGAS gene are operably linked to a promoter inducible under exogenous environmental conditions, e.g., conditions in the tumor microenvironment. In certain embodiments, the diadenylate cyclase gene and/or cGAS gene are operably linked to a promoter inducible by cumate or salicylate, or another chemical inducer. In certain embodiments, the diadenylate cyclase gene and/or cGAS gene are operably linked to a constitutive promoter. In one embodiment, the diadenylate cyclase gene and/or cGAS gene is operably linked to a promoter inducible under low oxygen conditions, e.g., an FNR promoter. In certain embodiments, one or more genetically engineered bacteria comprise gene sequence(s) encoding diadenylate cyclase gene, e.g., dacA, e.g., from Listeria monocytogenes, and cGAS, e.g., human cGAS, wherein the diadenylate cyclase gene and/or cGAS gene is operably linked to a promoter inducible by cumate or salicylate as described herein. In certain embodiments, the diadenylate cyclase and cGAS gene sequences are integrated into the bacterial chromosome. Suitable integration sites are described herein and known in the art. In certain embodiments, the bacteria comprising gene sequences encoding diadenylate cyclase and cGAS further comprise a mutation or deletion in dapA or thyA or both genes. In any of these embodiments, the bacteria may further comprise a prophage modification, e.g., a mutation or deletion, in an endogenous prophage. In one example, the prophage modification is a deletion of one or more genes, e.g., a 10 kb deletion. In a non-limiting example, the genetically engineered bacteria comprising gene sequences encoding diadenylate cyclase and cGAS are derived from E. coli Nissle and the prophage modification comprises a deletion or mutation in Nissle Phage 3, described herein.

In any of these embodiments describing genetically engineered bacteria comprising gene sequences encoding one or more diadenylate cyclases and cGAS producing polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases and cGAS polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one specific embodiment, one or more genetically engineered bacteria comprise gene sequence(s) encoding diadenylate cyclase e.g., DacA, e.g., from Listeria monocytogenes, and cGAS e.g., human cGAS, wherein the diadenylate cyclase gene and/or cGAS gene is operably linked to a promoter inducible under low oxygen conditions, e.g., an FNR promoter. The diadenylate cyclase gene and cGAS gene sequences are integrated into the bacterial chromosome. The bacteria further comprise an auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria comprising gene sequences encoding one or more diadenylate cyclases and cGAS polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively, the genetically engineered bacteria comprising gene sequences encoding one or more diadenylate cyclases and cGAS polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In any of these embodiments, the one or more bacteria genetically engineered to produce one or more STING agonists may be administered alone or in combination with one or more immune checkpoint inhibitors described herein, including but not limited to anti-CTLA4, anti-PD1, or anti-PD-L1 antibodies. In some embodiments, the one or more genetically engineered bacteria which produce STING agonists evoke immunological memory when administered in combination with checkpoint inhibitor therapy.

In any of these embodiments, the one or more bacteria genetically engineered to produce STING agonists may be genetically engineered to produce and secrete or display on their surface one or more immune checkpoint inhibitors described herein, including but not limited to anti-CTLA4, anti-PD1, or anti-PD-L1 antibodies. In some embodiments, the one or more genetically engineered bacteria which comprise gene sequences encoding one or more enzymes for STING agonist production and gene sequences encoding one or more immune checkpoint inhibitor antibodies, e.g., scFv antibodies, promote immunological memory upon rechallenge/reoccurrence of a tumor.

In any of these embodiments, the one or more bacteria genetically engineered to produce one or more STING agonists may be administered alone or in combination with one or more immune stimulatory agonists described herein, e.g., agonistic antibodies, including but not limited to anti-OX40, anti-41BB, or anti-GITR antibodies. In some embodiments, the one or more genetically engineered bacteria which produce STING agonists evoke immunological memory when administered in combination with anti-OX40, anti-41BB, or anti-GITR antibodies.

In any of these embodiments, the one or more bacteria genetically engineered to produce STING agonists may be genetically engineered to produce and secrete or display on their surface one or more immune stimulatory agonists described herein, e.g., agonistic antibodies, including but not limited to anti-OX40, anti-41BB, or anti-GITR antibodies. In some embodiments, the one or more genetically engineered bacteria comprising gene sequences encoding one or more STING agonist producing enzymes and gene sequences encoding one or more costimulatory antibodies, e.g., selected from anti-OX40, anti-41BB, or anti-GITR antibodies evoke immunological memory.

In one embodiment, administration of the STING agonist producing strain elicits an abscopal effect when administered alone or in combination with checkpoint inhibitor therapy and/or costimulatory antibodies, e.g., selected from anti-OX40, anti-41BB, or anti-GITR antibodies. In one embodiment, administration of genetically engineered bacteria comprising one or more genes encoding diadenylate cyclase, e.g., DacA, e.g., from Listeria monocytogenes, elicits an abscopal effect. In one embodiment, the abscopal effect is observed between day 2 and day 3. In one embodiment, administration of genetically engineered bacteria comprising one or more genes encoding cGAS, e.g., human cGAS, elicits an abscopal effect.

Also, in some embodiments, the genetically engineered bacteria and/or other microorganisms are further capable of expressing any one or more of the described circuits and further comprise one or more of the following: (1) one or more auxotrophies, such as any auxotrophies known in the art and provided herein, e.g., dapA and thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art (7) one or more circuits for the production or degradation of one or more metabolites (e.g., kynurenine, tryptophan, adenosine, arginine) described herein, (8) one or more immune initiators (e.g. STING agonist, CD40L, SIRPα) described herein, (9) one or more immune sustainers (e.g. IL-15, IL-12, CXCL10) described herein, and (10) combinations of one or more of such additional circuits.

### CD40

CD40 is a costimulatory protein found on antigen presenting cells and is required for their activation. The binding of CD154 (CD40L) on T helper cells to CD40 activates antigen presenting cells and induces a variety of downstream immunostimulatory effects. In some embodiments, the immune modulator is an agonist of CD40, for example, an agonist selected from an agonistic anti-CD40 antibody, agonistic anti-CD40 antibody fragment, CD40 ligand (CD40L) polypeptide, and CD40L polypeptide fragment. Thus, in some embodiments, the genetically engineered bacteria comprise sequence(s) encoding an agonistic anti-CD40 antibody or fragment thereof, or a CD40 ligand (CD40L) polypeptide or fragment thereof.

Thus, in some embodiments, the engineered bacteria is engineered to produce an agonistic anti-CD40 antibody or fragment thereof, or a CD40 ligand (CD40L) polypeptide or fragment thereof. In some embodiments, the engineered bacteria comprises sequence to encode an agonistic anti-CD40 antibody or fragment thereof, or a CD40 ligand (CD40L) polypeptide or fragment thereof. In some embodiments, the engineered bacteria comprise gene sequence encoding one or more copies of an antibody directed against CD40. In some embodiments, the CD40 is human CD40. In some embodiments, the anti-CD40 antibody is an scFv. In some embodiments, the anti-CD40 antibody is secreted. In some embodiments, the anti-CD40 antibody is displayed on the cell surface. In any of these embodiments, the gene sequences encoding the agonistic anti-CD40 antibody or fragment thereof, or a CD40 ligand (CD40L) polypeptide or fragment thereof further encode a secretion tag, e.g., as described herein.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CD40 ligand than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more CD40 ligand than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CD40 ligand than unmodified bacteria of the same bacterial subtype under the same conditions.

In any of these embodiments, the bacteria genetically engineered to produce CD40 ligand secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CD40 ligand than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more CD40 ligand than unmodified bacteria of the same bacterial subtype under the same conditions. In yet another embodiment, the genetically engineered bacteria secrete three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CD40 ligand than unmodified bacteria of the same bacterial subtype under the same conditions.

In some embodiments, the bacteria genetically engineered to secrete CD40 ligand are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete CD40 ligand are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete CD40 ligand are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CD40 ligand are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CD40 ligand are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CD40 ligand are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CD40 ligand are capable of increasing CCR7 expression on dendritic cells and/or macrophages.

In some embodiments, CCR7 is at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more induced as compared to an unmodified bacteria of the same subtype under the same conditions. In some embodiments, CCR7 is about 1.0-1.2-fold, 1.2-1.4-fold, 1.4-1.6-fold, 1.6-1.8-fold, 1.8-2-fold, or two-fold more induced than observed with than unmodified bacteria of the same bacterial subtype under the same conditions. In yet another embodiment, the CCR7 is about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold or more induced than observed with unmodified bacteria of the same bacterial subtype under the same conditions. In one embodiment, the levels of induced CCR7 in macrophages 25%-55%, about 30-45% greater than observed with unmodified bacteria of the same bacterial subtype under the same conditions.

In one embodiment, the levels of induced CCR7 in dendritic cells is about two fold greater than observed with unmodified bacteria of the same bacterial subtype under the same conditions.

In some embodiments, the bacteria genetically engineered to produce CD40 ligand are capable of increasing CCR7 expression on dendritic cells and/or macrophages.

In some embodiments, CD40 is at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more induced as compared to an unmodified bacteria of the same subtype under the same conditions. In some embodiments, CD40 is about 1.0-1.2-fold, 1.2-1.4-fold, 1.4-1.6-fold, 1.6-1.8-fold, 1.8-2-fold, or two-fold more induced than observed with than unmodified bacteria of the same bacterial subtype under the same conditions. In yet another embodiment, the CD40 is about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold or more induced than observed with unmodified bacteria of the same bacterial subtype under the same conditions. In one embodiment, the levels of induced CD40 in macrophages 30-50% greater than observed with unmodified bacteria of the same bacterial subtype under the same conditions.

In one embodiment, the levels of induced CD40 in dendritic cells is about 10% greater than observed with unmodified bacteria of the same bacterial subtype under the same conditions.

Accordingly, in one embodiment, the genetically engineered bacteria encode a CD40 Ligand polypeptide that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1093. In another embodiment, the polypeptide comprises SEQ ID NO: 1093. In yet another embodiment, the polypeptide expressed by the genetically engineered bacteria consists of SEQ ID NO: 1093.

In some embodiments, the genetically engineered microorganisms are capable of expressing any one or more of the described circuits encoding an agonistic anti-CD40 antibody or fragment thereof, or a CD40 ligand (CD40L) polypeptide or fragment thereof in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments, the gene sequences(s) are controlled by a promoter inducible by such conditions and/or inducers. In some embodiments such an inducer may be administered in vivo to induce effector gene expression. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described circuits are present on one or more plasmids (e.g., high copy or low copy) or are integrated into one or more sites in the microorganismal chromosome.

In any of these embodiments, the genetically engineered bacteria comprising gene sequence(s) encoding an agonistic anti-CD40 antibody or fragment thereof, or a CD40 ligand (CD40L) polypeptide or fragment thereof further comprise gene sequence(s) encoding one or more further effector molecule(s), i.e., therapeutic molecule(s) or a metabolic converter(s). In any of these embodiments, the circuit encoding an agonistic anti-CD40 antibody or fragment thereof, or a CD40 ligand (CD40L) polypeptide or fragment thereof may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding an agonistic anti-CD40 antibody or fragment thereof, or a CD40 ligand (CD40L) polypeptide or fragment thereof may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding an agonistic anti-CD40 antibody or fragment thereof, or a CD40 ligand (CD40L) polypeptide or fragment thereof encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding an agonistic anti-CD40 antibody or fragment thereof, or a CD40 ligand (CD40L) polypeptide or fragment thereof encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described circuits encoding an agonistic anti-CD40 antibody or fragment thereof, or a CD40 ligand (CD40L) polypeptide or fragment thereof and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### GMCSF

Granulocyte-macrophage colony-stimulating factor (GM-CSF), also known as colony stimulating factor 2 (CSF2), is a monomeric glycoprotein secreted by macrophages, T cells, mast cells, NK cells, endothelial cells and fibroblasts. GM-CSF is a white blood cell growth factor that functions as a cytokine, facilitating the development of the immune system and promoting defense against infections. For example, GM-CSF stimulates stem cells to produce granulocytes (neutrophils, eosinophils, and basophils) and monocytes, which monocytes exit the circulation and migrate into tissue, whereupon they mature into macrophages and dendritic cells. GM-CSF is part of the immune/inflammatory cascade, by which activation of a small number of macrophages rapidly lead to an increase in their numbers, a process which is crucial for fighting infection. GM-CSF signals via the signal transducer and activator of transcription, STATS or via STAT3 (which activates macrophages).

In some embodiments, the genetically engineered bacteria are capable of producing an immune modulator that modulates dendritic cell activation. In some embodiments, the immune modulator is GM-CSF. Thus, in some embodiments, the engineered bacteria is engineered to produce GM-CSF. In some embodiments, the engineered bacteria comprises sequence that encodes GM-CSF. In some embodiments, the engineered bacteria comprises sequence to encode GM-CSF and sequence to encode a secretory peptide(s) for the secretion of GM-CSF. Exemplary secretion tags and secretory methods are described herein.

In some embodiments, the genetically engineered microorganisms are capable of expressing any one or more of the described GM-CSF circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding GM-CSF are controlled by a promoter inducible by such conditions and/or inducers. In some embodiments, the gene sequences(s) encoding GM-CSF are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described genes sequences encoding GM-CSF are present on one or more plasmids (e.g., high copy or low copy) or are integrated into one or more sites in the microorganismal chromosome.

In any of these embodiments, the genetically engineered bacteria comprising gene sequence(s) encoding GM-CSF further comprise gene sequence(s) encoding one or more further effector molecule(s), i.e., therapeutic molecule(s) or a metabolic converter(s). In any of these embodiments, the circuit encoding GM-CSF may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding GM-CSF may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding GM-CSF encode DacA.

DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding GM-CSF encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In some embodiments, the genetically engineered microorganisms are capable of expressing any one or more of the described circuits encoding GM-CSF and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Activation and Priming of Effector Immune Cells (Immune Stimulators)

### T-Cell Activators

### Cytokines and Cytokine Receptors

CD4 (4) is a glycoprotein found on the surface of immune cells such as cells, monocytes, macrophages, and dendritic cells. CD4+T helper cells are white blood cells that function to send signals to other types of immune cells, thereby assisting other immune cells in immunologic processes, including maturation of B cells Into plasma cells and memory B cells, and activation of cytotoxic T cells and macrophages. T helper cells become activated when they are presented with peptide antigens by MHC class II molecules, which are expressed on the surface of antigen-presenting cells (APCs). Once activated, T helper cells divide and secrete cytokines that regulate or assist in the active immune response. T helper cells can differentiate into one of several subtypes, including TH1, TH2, TH3, TH17, TH9, or TFH cells, which secrete different cytokines to facilitate different types of immune responses.

Cytotoxic T cells (TC cells, or CTLs) destroy virus-infected cells and tumor cells, and are also implicated in transplant rejection. These cells are also known as CD8+ T cells since they express the CD8 glycoprotein at their surfaces. Cytotoxic T cells recognize their targets by binding to antigen associated with MHC class I molecules, which are present on the surface of all nucleated cells.

In some embodiments, the genetically engineered microorganisms, e.g., genetically engineered bacteria, are capable of producing one or more effector molecules or immune modulator, that modulates one or more T effector cells, e.g., CD4+ cell and/or CD8+ cell. In some embodiments, the genetically engineered bacteria are capable of producing one or more effector molecules that activate, stimulate, and/or induce the differentiation of one or more T effector cells, e.g., CD4+ and/or CD8+ cells. In some embodiments, the immune modulator is a cytokine that activates, stimulates, and/or induces the differentiation of a T effector cell, e.g., CD4+ and/or CD8+ cells. In some embodiments, the genetically engineered bacteria produce one or more cytokines selected from IL-2, IL-15, IL-12, IL-7, IL-21, IL-18, TNF, and IFN-gamma. As used herein, the production of one or more cytokines includes fusion proteins which comprise one or more cytokines, which are fused through a peptide linked to another cytokine or other immune modulatory molecule. Examples include but are not limited to IL-12 and IL-15 fusion proteins. In general, all agonists and antagonists described herein may be fused to another polypeptide of interest through a peptide linker, to improve or alter their function. For example, in some embodiments, the genetically engineered bacteria comprise sequence(s) encoding one or more cytokines selected from IL-2, IL-15, IL-12, IL-7, IL-21, IL-18, TNF, and IFN-gamma. “In some embodiments, the genetically engineered microorganisms encode one or more cytokine fusion proteins. Non-limiting examples of such fusion proteins include one or more cytokine polypeptides operably linked to an antibody polypeptide, wherein the antibody recognizes a tumor-specific antigen, thereby bringing the cytokine(s) into proximity with the tumor.

Interleukin 12 (IL-12) is a cytokine, the actions of which create an interconnection between the innate and adaptive immunity. IL-12 is secreted by a number of immune cells, including activated dendritic cells, monocytes, macrophages, and neutrophils, as well as other cell types. IL-12 is a heterodimeric protein (IL-12-p′70; IL-12-p35/p40) consisting of p35 and p40 subunits, and binds to a receptor composed of two subunits, IL-12R-β1 and IL-12R-β2. IL-12 receptor is expressed constitutively or inducibly on a number of immune cells, including NK cells, T, and B lymphocytes. Upon binding of IL-12, the receptor is activated and downstream signaling through the JAK/STAT pathway initiated, resulting in the cellular response to IL-12. IL-12 acts by increasing the production of IFN-γ, which is the most potent mediator of IL-12 actions, from NK and T cells. In addition, IL-12 promotes growth and cytotoxicity of activated NK cells, CD8+ and CD4+ T cells, and shifts the differentiation of CD4+Th0 cells toward the Th1 phenotype. Further, IL-12 enhances of antibody-dependent cellular cytotoxicity (ADCC) against tumor cells and the induction of IgG and suppression of IgE production from B cells. In addition, IL-12 also plays a role in reprogramming of myeloid-derived suppressor cells, directs the Th1-type immune response and helps increase expression of MHC class I molecules (e.g., reviewed in Waldmann et al., Cancer Immunol Res March 2015 3; 219).

Thus, in some embodiments, the engineered bacteria is engineered to produce IL-12. In some embodiments, the engineered bacteria comprises sequence to encode IL-12 (i.e., the p35 and p40 subunits). In some embodiments, the engineered bacteria is engineered to over-express IL-12, for example, operatively linked to a strong promoter and/or comprising more than one copy of the IL-12 gene sequence. In some embodiments, the engineered bacteria comprises sequence(s) encoding two or more copies of IL-12, e.g., two, three, four, five, six or more copies of IL-12 gene. In some embodiments, the engineered bacteria produce one or more immune modulators that stimulate the production of IL-12. In some embodiments, the engineered bacteria comprises sequence to encode IL-12 and sequence to encode a secretory peptide(s) for the secretion of IL-12.

In some embodiments, the genetically engineered bacteria comprise a gene sequence in which two interleukin-12 monomer subunits (IL-12A (p35) and IL-12B (p40)) is covalently linked by a linker In some embodiments, the linker is a serine glycine rich linker. In one embodiment, the gene sequence encodes construct in which a 15 amino acid linker of ‘GGGGSGGGGSGGGGS’ (SEQ ID NO: 1247) is inserted between two monomer subunits (IL-12A (p35) and IL-12B (p40) to produce a forced dimer human IL-12 (diIL-12) fusion protein. In some embodiments, the gene sequence is codon optimized for expression, e.g., for expression in E. coli. In any of the embodiments, in which the genetically engineered bacteria comprise a gene sequence for the expression of IL-12, in which the two subunits are linked, the gene sequence may further comprise a secretion tag. The secretion tag includes any of the secretion tags described herein or known in the art.

In some embodiments, the genetically engineered bacteria comprise a gene sequence encoding a IL-12 (p35) subunit linked to the IL-12 (p40) subunit having at least about 80% identity with a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194. In some embodiments, the genetically engineered bacteria comprise a gene sequence encoding a IL-12 (p35) subunit linked to the IL-12 (p40) subunit that has about having at least about 90% identity with a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194. In some embodiments, the genetically engineered bacteria comprise a gene sequence encoding a IL-12 (p35) subunit linked to the IL-12 (p40) subunit that has about having at least about 95% identity with a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194. In some embodiments, the genetically engineered bacteria comprise a gene sequence encoding a IL-12 (p35) subunit linked to the IL-12 (p40) subunit that has about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194, or a functional fragment thereof. In another embodiment, the IL-12 (p35) subunit linked to the IL-12 (p40) subunit comprises a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194. In yet another embodiment, the IL-12 (p35) subunit linked to the IL-12 (p40) subunit expressed by the genetically engineered bacteria consists of a sequence selected from SEQ ID NO: 1169, SEQ ID NO: 1170, SEQ ID NO: 1171, SEQ ID NO: 1172, SEQ ID NO: 1173, SEQ ID NO: 1174, SEQ ID NO: 1175, SEQ ID NO: 1176, SEQ ID NO: 1177, SEQ ID NO: 1178, SEQ ID NO: 1179, SEQ ID NO: 1191, SEQ ID NO: 1192, SEQ ID NO: 1193, and SEQ ID NO: 1194. In any of these embodiments wherein the genetically engineered bacteria encode IL-12 (p35) subunit linked to the IL-12 (p40) subunit, one or more of the sequences encoding a Tag, such as V5, FLAG or His Tags, are removed. In other embodiments, the secretion tag is removed and replaced by a different secretion tag.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IL-12 than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more IL-12 than unmodified bacteria of the same bacterial subtype under the same conditions. In yet another embodiment, the genetically engineered bacteria produce at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IL-12 than unmodified bacteria of the same bacterial subtype under the same conditions.

In any of these embodiments, the genetically engineered bacteria produce at least about 5-10, 10-20, 20-30, 30-40, 40-50, 50-60, 60-70, 80-90, 90-100, 100-150, 150-200, 200-250, 250-300, 300-350, 350-400 pg/ml of media, e.g., after 4 hours of induction. In one embodiment, the genetically engineered bacteria produce at least about 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500, pg/ml of media, e.g., after 4 hours of induction.

In any of these embodiments, the bacteria genetically engineered to produce IL-12 secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IL-12 than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more IL-12 than unmodified bacteria of the same bacterial subtype under the same conditions. In yet another embodiment, the genetically engineered bacteria secrete at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IL-12 than unmodified bacteria of the same bacterial subtype under the same conditions.

In some embodiments, the bacteria genetically engineered to secrete IL-12 are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete IL-12 are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete IL-12 are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IL-12 are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to IL-12 are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IL-12 are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

IL-15 displays pleiotropic functions in homeostasis of both innate and adaptive immune system and binds to IL-15 receptor, a heterotrimeric receptor composed of three subunits. The alpha subunit is specific for IL-15, while beta (CD122) and gamma (CD132) subunits are shared with the IL-2 receptor, and allow shared signaling through the JAK/STAT pathways. IL-15 is produced by several cell types, including dendritic cells, monocytes and macrophages. Co-expression of IL-15Rα and IL-15 produced in the same cell, allows intracellular binding of IL-15 to IL-15Rα, which is then shuttled to the cell surface as a complex. Once on the cell surface, then, the IL-15Rα of these cells is able to trans-present IL-15 to IL-15Rβ-γc of CD8 T cells, NK cells, and NK-T cells, which do not express IL-15, inducing the formation of the so-called immunological synapse. Murine and human IL-15Rα, exists both in membrane bound, and also in a soluble form. Soluble IL-15Rα (sIL-15Rα) is constitutively generated from the transmembrane receptor through proteolytic cleavage.

IL-15 is critical for lymphoid development and peripheral maintenance of innate immune cells and immunological memory of T cells, in particular natural killer (NK) and CD8+ T cell populations. In contrast to IL-2, IL-15 does not promote the maintenance of Tregs and furthermore, IL-15 has been shown to protect effector T cells from IL-2-mediated activation-induced cell death.

Consequently, delivery of IL-15 is considered a promising strategy for long-term anti-tumor immunity. In a first-in-human clinical trial of recombinant human IL-15, a 10-fold expansion of NK cells and significantly increased the proliferation of γδT cells and CD8+ T cells was observed upon treatment. In addition, IL-15 superagonists containing cytokine-receptor fusion complexes have been developed and are evaluated to increase the length of the response. These include the L-15 N72D superagonist/IL-15RαSushi-Fc fusion complex (IL-15SA/IL-15RαSu-Fc; ALT-803) (Kim et al., 2016 IL-15 superagonist/IL-15RαSushi-Fc fusion complex (IL-15SA/IL-15RαSu-Fc; ALT-803) markedly enhances specific subpopulations of NK and memory CD8+ T cells, and mediates potent anti-tumor activity against murine breast and colon carcinomas).

Thus, in some embodiments, the engineered bacteria is engineered to produce IL-15. In some embodiments, IL-15 is secreted.

The biological activity of IL-15 is greatly improved by pre-associating IL-15 with a fusion protein IL-15Rα-Fc or by direct fusion with the sushi domain of IL-15Rα (hyper-IL-15) to mimic trans-presentation of IL-15 by cell-associated IL-15Rα. IL-15, either administrated alone or as a complex with IL-15Rα, exhibits potent antitumor activities in animal models (Cheng et al., Immunotherapy of metastatic and autochthonous liver cancer with IL-15/IL-15Rα fusion protein; Oncoimmunology. 2014; 3(11): e963409, and references therein).

In some embodiments, the engineered bacteria comprises gene sequences encoding IL-15. In some embodiments, the engineered bacteria comprises sequence to encode IL-15Rα. In some embodiments, the engineered bacteria comprises sequence to encode IL-15 and sequence to encode IL-15Rα. In some embodiments, the engineered bacteria comprises sequence to encode a fusion polypeptide comprising IL-15 and IL-15Rα. In some embodiments, the engineered bacteria comprises sequence(s) encoding IL-15 and sequence encoding secretion tag. Exemplary secretion tags are known in the art and described herein.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IL-15 or IL-15/IL-15Rα fusion protein than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more IL-15 or IL-15/IL-15Rα fusion protein than unmodified bacteria of the same bacterial subtype under the same conditions. In yet another embodiment, the genetically engineered bacteria produce at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IL-15 or IL-15/IL-15Rα fusion protein than unmodified bacteria of the same bacterial subtype under the same conditions.

In any of these embodiments, the bacteria genetically engineered to produce IL-15 or IL-15/IL-15Rα fusion protein secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IL-15 or IL-15/IL-15Rα fusion protein than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more IL-15 or IL-15/IL-15Rα fusion protein than unmodified bacteria of the same bacterial subtype under the same conditions. In yet another embodiment, the genetically engineered bacteria secrete at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IL-15 or IL-15/IL-15Rα fusion protein than unmodified bacteria of the same bacterial subtype under the same conditions.

In some embodiments, the bacteria genetically engineered to secrete IL-15 or IL-15/IL-15Rα fusion protein are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete IL-15 or IL-15/IL-15Rα fusion protein are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete IL-15 or IL-15/IL-15Rα fusion protein are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IL-15 or IL-15/IL-15Rα fusion protein are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IL-15 or IL-15/IL-15Rα fusion protein are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IL-15 or IL-15/IL-15Rα fusion protein are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to produce IL-15 or IL-15/IL-15Rα fusion protein are capable of promoting expansion of NK cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In yet another embodiment, the genetically engineered bacteria promote the expansion of NK cells to at least 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria promote the expansion of NK cells to a at least three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than bacteria of the same bacterial subtype under the same conditions.

In some embodiments, the bacteria genetically engineered to produce IL-15 or IL-15/IL-15Rα fusion protein are capable of increasing the proliferation of γδT cells and/or CD8+ T cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In yet another embodiment, the genetically engineered bacteria increase the proliferation of γδT cells and/or CD8+ T cells by at least 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria increasing the proliferation of γδT cells and/or CD8+ T cells at least three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce IL-15 or IL-15/IL-15Rα fusion protein are capable of binding to IL-15 or IL-15/IL-15Rα fusion protein receptor by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater affinity as compared to an unmodified bacteria of the same subtype under the same conditions. In yet another embodiment, the genetically engineered bacteria bind to IL-15 or IL-15/IL-15Rα fusion protein receptor with at least 1.0-1.2-fold, 1.2-1.4-fold, 1.4-1.6-fold, 1.6-1.8-fold, 1.8-2-fold, or two-fold greater affinity than unmodified bacteria of the same bacterial subtype under the same conditions. In yet another embodiment, the genetically engineered bacteria are capable of binding to IL-15 or IL-15/IL-15Rα fusion protein receptor with at least three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold or greater affinity than unmodified bacteria of the same bacterial subtype under the same conditions.

In some embodiments, the genetically engineered bacteria comprising one or more genes encoding IL-15 for secretion are capable of inducing STATS phosphorylation, e.g., in CD3+IL15RAalpha+ T-cells. In some embodiments, the bacteria genetically engineered to produce IL-15 or IL-15/IL-15Rα fusion protein are capable of inducing STATS phosphorylation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more to higher levels as compared to an unmodified bacteria of the same subtype under the same conditions. In yet another embodiment, the genetically engineered bacteria induce STATS phosphorylation with at least 1.0-1.2-fold, 1.2-1.4-fold, 1.4-1.6-fold, 1.6-1.8-fold, 1.8-2-fold, or two-fold or more to higher levels than unmodified bacteria of the same bacterial subtype under the same conditions. In yet another embodiment, the genetically engineered bacteria induce STATS phosphorylation with at least three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold or more higher levels than unmodified bacteria of the same bacterial subtype under the same conditions. In one embodiment, the IL-15 secreting strain induce STATS phosphorylation comparable to that of rhIL15 at the same amount under the same conditions.

In some embodiments, the genetically engineered bacteria comprising one or more genes encoding IL-15 for secretion are capable of inducing STAT3 phosphorylation, e.g., in CD3+IL15RAalpha+ T-cells. In some embodiments, the genetically engineered bacteria comprising one or more genes encoding IL-15 for secretion are capable of inducing STAT3 phosphorylation, e.g., in CD3+IL15RAalpha+ T-cells. In some embodiments, the bacteria genetically engineered to produce IL-15 or IL-15/IL-15Rα fusion protein are capable of inducing STAT3 phosphorylation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more to higher levels as compared to an unmodified bacteria of the same subtype under the same conditions. In yet another embodiment, the genetically engineered bacteria induce STAT3 phosphorylation with at least 1.0-1.2-fold, 1.2-1.4-fold, 1.4-1.6-fold, 1.6-1.8-fold, 1.8-2-fold, or two-fold or more to higher levels than unmodified bacteria of the same bacterial subtype under the same conditions. In yet another embodiment, the genetically engineered bacteria induce STAT3 phosphorylation with at least three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold or more higher levels than unmodified bacteria of the same bacterial subtype under the same conditions. In one embodiment, the IL-15 secreting strain induce STAT3 phosphorylation comparable to that of rhIL15 at the same amount under the same conditions.

In some embodiments, the genetically engineered bacteria comprise gene sequence(s) encoding one or more IL-15, IL-Ralpha, Linker, and IL-15-IL15Ralpha fusion polypeptide(s) having at least about 80% identity with a sequence selected from SEQ ID NO: 1133, SEQ ID NO: 1134, SEQ ID NO: 1135, SEQ ID NO: 1136. In some embodiments, the genetically engineered bacteria comprise gene sequence(s) encoding one or more IL-15, IL-Ralpha, Linker, and IL-15-IL15Ralpha fusion polypeptide(s) having at least about 90% identity with a sequence selected from SEQ ID NO: 1133, SEQ ID NO: 1134, SEQ ID NO: 1135, SEQ ID NO: 1136. In some embodiments, the genetically engineered bacteria comprise gene sequence(s) encoding one or more IL-15, IL-Ralpha, Linker, and IL-15-IL15Ralpha fusion polypeptide(s) having at least about 90% identity with a sequence selected from SEQ ID NO: 1133, SEQ ID NO: 1134, SEQ ID NO: 1135, SEQ ID NO: 1136.

In some embodiments, genetically engineered bacteria comprise a gene sequence encoding a polypeptide that is at least about 80%, at least about 85%, at least about 90%, at least about 95%, or at least about 99% identity to one or more polypeptide(s) selected from SEQ ID NO: 1133, SEQ ID NO: 1134, SEQ ID NO: 1135, SEQ ID NO: 1136 or a functional fragment thereof. In other specific embodiments, the polypeptide consists of one or more polypeptide(s) selected from SEQ ID NO: 1133, SEQ ID NO: 1134, SEQ ID NO: 1135, SEQ ID NO: 1136.

In some embodiments, the genetically engineered bacteria comprise a gene sequence encoding IL-15, IL-Ralpha, Linker, and IL-15-IL15Ralpha fusion protein, or a fragment or functional variant thereof. In one embodiment, the gene sequence encoding IL-15 or IL-15 fusion protein has at least about 90% identity with a sequence selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344. In one embodiment, the gene sequence encoding IL-15 or IL-15 fusion protein has at least about 80% identity with a sequence selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344. In one embodiment, the gene sequence encoding IL-15 or IL-15 fusion protein has at least about 95% identity with a sequence selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344. In certain embodiments, the IL-15, IL-Ralpha, Linker, and IL-15-IL15Ralpha fusion protein sequence has at least about 80%, 85%, 86%, 87%, 88%, 89%, 90%, 91%, 92%, 93%, 94%, 95%, 96%, 97%, 98%, or 99% identity with one or more polynucleotides selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344 or functional fragments thereof. In some specific embodiments, the gene sequence comprises one or more polynucleotides selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344. In other specific embodiments, the gene sequence consists of one or more polynucleotides selected from SEQ ID NO: 1338, SEQ ID NO: 1339, SEQ ID NO: 1340, SEQ ID NO: 1341, SEQ ID NO: 1342, SEQ ID NO: 1343, SEQ ID NO: 1344.

In some embodiments, the genetically engineered bacteria comprise a gene sequence encoding IL-15 or IL-15 fusion protein, or a fragment or functional variant thereof. In one embodiment, the gene sequence encoding IL-15 or IL-15 fusion protein has at least about 80% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In another embodiment, the gene sequence encoding IL-15 or IL-15 fusion protein has at least about 85% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In one embodiment, the gene sequence encoding IL-15 or IL-15 fusion protein has at least about 90% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In one embodiment, the gene sequence IL-15 or IL-15 fusion protein has at least about 95% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In another embodiment, the gene sequence encoding IL-15 or IL-15 fusion protein has at least about 96%, 97%, 98%, or 99% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. Accordingly, in one embodiment, the gene sequence encoding IL-15 or IL-15 fusion protein has at least about 80%, 81%, 82%, 83%, 84%, 85%, 86%, 87%, 88%, 89%, 90%, 91%, 92%, 93%, 94%, 95%, 96%, 97%, 98%, or 99% identity with a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In another embodiment, the gene sequence encoding IL-15 or IL-15 fusion protein comprises a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In yet another embodiment, the gene sequence encoding IL-15 or IL-15 fusion protein consists of a sequence selected from SEQ ID NO: 1345, SEQ ID NO: 1200, SEQ ID NO: 1201, SEQ ID NO: 1202, SEQ ID NO: 1203, SEQ ID NO: 1204, and SEQ ID NO: 1199. In any of these embodiments wherein the genetically engineered bacteria encode IL-15 or IL-15 fusion protein, one or more of the sequences encoding a Tag are removed.

In some embodiments, the genetically engineered bacteria comprise a gene sequence encoding a IL-15 or IL-15 fusion protein described herein having at least about 80% identity with a sequence selected from SEQ ID NO: 1195, SEQ ID NO: 1196, SEQ ID NO: 1197, and SEQ ID NO: 1198. In some embodiments, the genetically engineered bacteria comprise a gene sequence encoding a IL-15 or IL-15 fusion protein that has about having at least about 90% identity with a sequence selected from SEQ ID NO: 1195, SEQ ID NO: 1196, SEQ ID NO: 1197, and SEQ ID NO: 1198. In some embodiments, the genetically engineered bacteria comprise a gene sequence encoding a IL-15 or IL-15 fusion protein that has about having at least about 95% identity with a sequence selected from SEQ ID NO: 1195, SEQ ID NO: 1196, SEQ ID NO: 1197, and SEQ ID NO: 1198. In some embodiments, the genetically engineered bacteria comprise a gene sequence encoding a IL-15 or IL-15 fusion protein that has about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NO: 1195, SEQ ID NO: 1196, SEQ ID NO: 1197, and SEQ ID NO: 1198, or a functional fragment thereof. In another embodiment, the IL-15 or IL-15 fusion protein comprises a sequence selected from SEQ ID NO: 1195, SEQ ID NO: 1196, SEQ ID NO: 1197, and SEQ ID NO: 1198. In yet another embodiment, the IL-15 or IL-15 fusion protein expressed by the genetically engineered bacteria consists of a sequence selected from SEQ ID NO: 1195, SEQ ID NO: 1196, SEQ ID NO: 1197, and SEQ ID NO: 1198. In any of these embodiments wherein the genetically engineered bacteria encode IL-15 or IL-15 fusion protein, the secretion tag may be removed and replaced by a different secretion tag.

Interferon gamma (IFNγ or type II interferon), is a cytokine that is critical for innate and adaptive immunity against viral, some bacterial and protozoal infections. IFNγ activates macrophages and induces Class II major histocompatibility complex (MHC) molecule expression. IFNγ can inhibit viral replication and has immunostimulatory and immunomodulatory effects in the immune system. IFNγ is produced predominantly by natural killer (NK) and natural killer T (NKT) cells as part of the innate immune response, and by CD4 Th1 and CD8 cytotoxic T lymphocyte (CTL) effector T cells. Once antigen-specific immunity develops IFNγ is secreted by T helper cells (specifically, Th1 cells), cytotoxic T cells (TC cells) and NK cells only. It has numerous immunostimulatory effects and plays several different roles in the immune system, including the promotion of NK cell activity, increased antigen presentation and lysosome activity of macrophages, activation of inducible Nitric Oxide Synthase iNOS, production of certain IgGs from activated plasma B cells, promotion of Th1 differentiation that leads to cellular immunity. It can also cause normal cells to increase expression of class I MHC molecules as well as class II MHC on antigen-presenting cells, promote adhesion and binding relating to leukocyte migration, and is involved in granuloma formation through the activation of macrophages so that they become more powerful in killing intracellular organisms.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IFN-gamma than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more IFN-gamma than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IFN-gamma than unmodified bacteria of the same bacterial subtype under the same conditions.

In any of these embodiments, the bacteria genetically engineered to produce IFN-gamma secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more IFN-gamma than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more IFN-gamma than unmodified bacteria of the same bacterial subtype under the same conditions. In yet another embodiment, the genetically engineered bacteria secrete three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more IFN-gamma than unmodified bacteria of the same bacterial subtype under the same conditions.

In some embodiments, the bacteria genetically engineered to secrete IFN-gamma are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria comprising one or more genes encoding IFN-gamma induce STAT1 phosphorylation in macrophage cell lines. In any of these embodiments, the bacteria genetically engineered to produce IFN-gamma induce STAT1 phosphorylation 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% or greater levels than unmodified bacteria of the same bacterial subtype under the same conditions. In yet another embodiment, the genetically engineered bacteria induce STAT1 phosphorylation 1.0-1.2-fold, 1.2-1.4-fold, 1.4-1.6-fold, 1.6-1.8-fold, 1.8-2-fold, or two-fold or greater levels than unmodified bacteria of the same bacterial subtype under the same conditions. In yet another embodiment, the genetically engineered bacteria induce STAT1 phosphorylation three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold or greater levels than unmodified bacteria of the same bacterial subtype under the same conditions.

In one specific embodiment, the bacteria are capable of increasing IFNgamma production in the tumor by 0.1, 0.2, 0.3 ng per gram of tumor relative to same bacteria unmodified bacteria of the same bacterial subtype under the same conditions. In one specific embodiment, the bacteria are capable of increasing IFNgamma production about 5, 10, or 15 fold relative to same bacteria unmodified bacteria of the same bacterial subtype under the same conditions.

In some embodiments, the bacteria genetically engineered to secrete IFN-gamma are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete IFN-gamma are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IFN-gamma are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IFN-gamma are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce IFN-gamma are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

Interleukin-18 (IL-18, also known as interferon-gamma inducing factor) is a proinflammatory cytokine that belongs to the IL-1 superfamily and is produced by macrophages and other cells. IL-18 binds to the interleukin-18 receptor, and together with IL-12 it induces cell-mediated immunity following infection with microbial products like lipopolysaccharide (LPS). Upon stimulation with IL-18, natural killer (NK) cells and certain T helper type 1 cells release interferon-γ (IFN-γ) or type II interferon, which plays a role in activating the macrophages and other immune cells. IL-18 is also able to induce severe inflammatory reactions.

Thus, in some embodiments, the engineered bacteria is engineered to produce IL-18. In some embodiments, the engineered bacteria comprises sequence to encode IL-18. In some embodiments, the engineered bacteria is engineered to over-express IL-18, for example, operatively linked to a strong promoter and/or comprising more than one copy of the IL-18 gene sequence. In some embodiments, the engineered bacteria comprises sequence(s) encoding two or more copies of IL-18 gene, e.g., two, three, four, five, six or more copies of IL-18 gene. In some embodiments, the genetically engineered bacterium expresses IL-18 and/or expresses secretory peptides under the control of a promoter that is activated by low-oxygen conditions. In some embodiments, the genetically engineered bacterium is a bacterium that expresses IL-18, and/or expresses secretory peptide(s) under the control of a promoter that is activated by low-oxygen conditions. In certain embodiments, the genetically engineered bacteria express IL-18 and/or secretory peptide(s), under the control of a promoter that is activated by hypoxic conditions, or by inflammatory conditions, such as any of the promoters activated by said conditions and described herein. In some embodiments, the genetically engineered bacteria expresses IL-18 and/or expresses secretory peptide(s), under the control of a cancer-specific promoter, a tissue-specific promoter, or a constitutive promoter, such as any of the promoters described herein.

Interleukin-2 (IL-2) is cytokine that regulates the activities of white blood cells (leukocytes, often lymphocytes). IL-2 is part of the body's natural response to microbial infection, and in discriminating between foreign (“non-self”) and “self”. IL-2 mediates its effects by binding to IL-2 receptors, which are expressed by lymphocytes. IL-2 is a member of a cytokine family, which also includes IL-4, IL-7, IL-9, IL-15 and IL-21. IL-2 signals through the IL-2 receptor, a complex consisting of alpha, beta and gamma sub-units. The gamma sub-unit is shared by all members of this family of cytokine receptors. IL-2 promotes the differentiation of T cells into effector T cells and into memory T cells when the initial T cell is stimulated by an antigen. Through its role in the development of T cell immunologic memory, which depends upon the expansion of the number and function of antigen-selected T cell clones, it also has a key role in cell-mediated immunity. IL-2 has been approved by the Food and Drug Administration (FDA) and in several European countries for the treatment of cancers (malignant melanoma, renal cell cancer). IL-2 is also used to treat melanoma metastases and has a high complete response rate.

Thus, in some embodiments, the engineered bacteria is engineered to produce IL-2. In some embodiments, the engineered bacteria comprises sequence to encode IL-2. In some embodiments, the engineered bacteria is engineered to over-express IL-2, for example, operatively linked to a strong promoter and/or comprising more than one copy of the IL-2 gene sequence. In some embodiments, the engineered bacteria comprises sequence(s) encoding two or more copies of IL-2 gene, e.g., two, three, four, five, six or more copies of IL-2 gene. In some embodiments, the genetically engineered bacterium expresses IL-2 and/or expresses secretory peptides under the control of a promoter that is activated by low-oxygen conditions. In some embodiments, the genetically engineered bacterium is a bacterium that expresses IL-2, and/or expresses secretory peptide(s) under the control of a promoter that is activated by low-oxygen conditions. In certain embodiments, the genetically engineered bacteria express IL-2 and/or secretory peptide(s), under the control of a promoter that is activated by hypoxic conditions, or by inflammatory conditions, such as any of the promoters activated by said conditions and described herein. In some embodiments, the genetically engineered bacteria expresses IL-2 and/or expresses secretory peptide(s), under the control of a cancer-specific promoter, a tissue-specific promoter, or a constitutive promoter, such as any of the promoters described herein.

Interleukin-21 is a cytokine that has potent regulatory effects on certain cells of the immune system, including natural killer(NK) cells and cytotoxic T cells. IL-21 induces cell division/proliferation in its these cells. IL-21 is expressed in activated human CD4+ T cells but not in most other tissues. In addition, IL-21 expression is up-regulated in Th2 and Th17 subsets of T helper cells. IL-21 is also expressed in NK T cells regulating the function of these cells. When bound to IL-21, the IL-21 receptor acts through the Jak/STAT pathway, utilizing Jak1 and Jak3 and a STAT3 homodimer to activate its target genes. IL-21 has been shown to modulate the differentiation programming of human T cells by enriching for a population of memory-type CTL with a unique CD28+CD127hi CD45RO+ phenotype with IL-2 producing capacity. IL-21 also has anti-tumor effects through continued and increased CD8+ cell response to achieve enduring tumor immunity. IL-21 has been approved for Phase 1 clinical trials in metastatic melanoma (MM) and renal cell carcinoma (RCC) patients.

Thus, in some embodiments, the engineered bacteria is engineered to produce IL-21. In some embodiments, the engineered bacteria comprises sequence that encodes IL-21. In some embodiments, the engineered bacteria is engineered to over-express IL-21, for example, operatively linked to a strong promoter and/or comprising more than one copy of the IL-21 gene sequence. In some embodiments, the engineered bacteria comprises sequence(s) encoding two or more copies of IL-21, e.g., two, three, four, five, six or more copies of IL-21 gene. In some embodiments, the engineered bacteria produce one or more immune modulators that stimulate the production of IL-21. In some embodiments, the engineered bacteria comprises sequence to encode IL-21 and sequence to encode a secretory peptide(s) for the secretion of 11-21. In some embodiments, the genetically engineered bacterium expresses IL-21 and/or expresses secretory peptides under the control of a promoter that is activated by low-oxygen conditions. In some embodiments, the genetically engineered bacterium is a bacterium that expresses 11-21, and/or expresses secretory peptide(s) under the control of a promoter that is activated by low-oxygen conditions. In certain embodiments, the genetically engineered bacteria express IL-21 and/or secretory peptide(s), under the control of a promoter that is activated by hypoxic conditions, or by inflammatory conditions, such as any of the promoters activated by said conditions and described herein. In some embodiments, the genetically engineered bacteria expresses IL-21 and/or expresses secretory peptide(s), under the control of a cancer-specific promoter, a tissue-specific promoter, or a constitutive promoter, such as any of the promoters described herein.

Tumor necrosis factor (TNF) (also known as cachectin or TNF alpha) is a cytokine that can cause cytolysis of certain tumor cell lines and can stimulate cell proliferation and induce cell differentiation under certain conditions. TNF is involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. The primary role of TNF is in the regulation of immune cells.

TNF can bind two receptors, TNFR1 (TNF receptor type 1; CD120a; p55/60) and TNFR2 (TNF receptor type 2; CD120b; p75/80). TNFR1 is expressed in most tissues, and can be fully activated by both the membrane-bound and soluble trimeric forms of TNF, whereas TNFR2 is found only in cells of the immune system, and respond to the membrane-bound form of the TNF homotrimer. Upon binding to its receptor, TNF can activate NF-κB and MAPK pathways which mediate the transcription of numerous proteins and mediate several pathways involved in cell differentiation and proliferation, including those pathways involved in the inflammatory response. TNF also regulates pathways that induce cell apoptosis.

In some embodiments, the genetically engineered bacteria are capable of producing an immune modulator that modulates dendritic cell activation. In some embodiments, the immune modulator is TNF. Thus, in some embodiments, the engineered bacteria is engineered to produce TNF. IN some embodiments, TNF is secreted from the bacterium, as described herein. In some embodiments, the engineered bacteria comprises sequence that encodes TNF.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more TNF than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more TNF than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more TNF than unmodified bacteria of the same bacterial subtype under the same conditions.

In any of these embodiments, the bacteria genetically engineered to produce TNF secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more TNF than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more TNF than unmodified bacteria of the same bacterial subtype under the same conditions. In yet another embodiment, the genetically engineered bacteria secrete three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more TNF than unmodified bacteria of the same bacterial subtype under the same conditions.

In some embodiments, the bacteria genetically engineered to secrete TNF are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete TNF are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete TNF are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce TNF are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In one embodiment, the genetically engineered bacteria are capable of reducing tumor volume by about 40-60%, by about 45-55%, e.g., on day 7 of a two dose treatment regimen. In one embodiment, tumor volume is about 300 mm3 upon administration of the bacteria expressing TNF, relative to about 600 mm3 upon administration of unmodified bacteria of the same subtype under the same conditions. In some embodiments, the bacteria genetically engineered to produce TNF are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce TNF are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to produce TNF are capable of increasing CCR7 expression on dendritic cells and/or macrophages.

In some embodiments, the genetically engineered bacteria comprising one or more genes encoding TNFα for secretion are capable of activating the NFkappaB pathway, e.g., in cells with TNF receptor. In some embodiments, the genetically engineered bacteria comprising one or more genes encoding TNFα are capable of inducing IkappaBalpha degradation. In some embodiments, secreted TNFα levels secreted from the engineered bacteria causes IkappaBalpha degradation to about the same extent as recombinant TNFα at the same concentration under the same conditions.

In some embodiments, the genetically engineered microorganisms are capable of expressing any one or more of the described IL-2, IL-15, IL-12, IL-7, IL-21, IL-18, TNF, and IFN-gamma circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding IL-2, IL-15, IL-12, IL-7, IL-21, IL-18, TNF, and IFN-gamma are controlled by a promoter inducible by such conditions and/or inducers. In some embodiments, the gene sequences(s) encoding IL-2, IL-15, IL-12, IL-7, IL-21, IL-18, TNF, and IFN-gamma are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described genes sequences encoding IL-2, IL-15, IL-12, IL-7, IL-21, IL-18, TNF, and/or IFN-gamma are present on one or more plasmids (e.g., high copy or low copy) or are integrated into one or more sites in the microorganismal chromosome.

In any of these embodiments, the genetically engineered bacteria comprising gene sequence(s) encoding IL-2, IL-15, IL-12, IL-7, IL-21, IL-18, TNF, and/or IFN-gamma further comprise gene sequence(s) encoding one or more further effector molecule(s), i.e., therapeutic molecule(s) or a metabolic converter(s). In any of these embodiments, the circuit encoding IL-2, IL-15, IL-12, IL-7, IL-21, IL-18, TNF, and/or IFN-gamma may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding IL-2, IL-15, IL-12, IL-7, IL-21, IL-18, TNF, and/or IFN-gamma may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding IL-2, IL-15, IL-12, IL-7, IL-21, IL-18, TNF, and/or IFN-gamma encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding IL-2, IL-15, IL-12, IL-7, IL-21, IL-18, TNF, and/or IFN-gamma comprise cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described circuits encoding IL-2, IL-15, IL-12, IL-7, IL-21, IL-18, TNF, and IFN-gamma and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Co-Stimulatory Molecules

Glucocorticoid-induced tumour necrosis factor receptor (TNFR)-related receptor (GITR, TNFR18) is a type I transmembrane protein and a member of the TNFR superfamily.1 GITR is expressed at high levels, predominantly, on CD25+CD4+ regulatory T (Treg) cells, but it is also constitutively expressed at low levels on conventional CD25− CD4+ and CD8+ T cells and is rapidly upregulated after activation. In vitro studies using an agonistic anti-GITR monoclonal antibody (mAb; DTA-1)2,6,7 or GITRL transfectants and soluble GITRL5,8,9 have shown that the GITR-GITRL pathway induces positive costimulatory signals leading to the activation of CD4+ and CD8+ effector T cells (as well as Treg cells, despite their opposing effector functions) (Piao et al., (2009) Enhancement of T-cell-mediated anti-tumour immunity via the ectopically expressed glucocorticoid-induced tumour necrosis factor receptor-related receptor ligand (GITRL) on tumours; Immunology, 127, 489-499, and references therein). In some embodiments, the effector or immune modulator, is an agonist of GITR, for example, an agonist selected from agonistic anti-GITR antibody, agonistic anti-GITR antibody fragment, GITR ligand polypeptide (GITRL), and GITRL polypeptide fragment. Thus, in some embodiments, the genetically engineered bacteria comprise sequence(s) encoding an agonistic anti-GITR antibody or fragment thereof, or a GITR ligand polypeptide or fragment thereof. Thus, in some embodiments, the engineered bacteria is engineered to produce an agonistic anti-GITR antibody or fragment thereof, or a GITR ligand polypeptide or fragment thereof. In some embodiments, the engineered bacteria comprises sequence to encode an agonistic anti-GITR antibody or fragment thereof, or a GITR ligand polypeptide or fragment thereof. In some embodiments, the engineered bacteria comprises sequence(s) to encode an agonistic anti-GITR antibody or fragment thereof, or a GITR ligand polypeptide or fragment thereof, and sequence to encode a secretory peptide(s) for the secretion of said antibodies and polypeptides. Non-limiting examples of secretion tags and suitable secretion mechanisms are described herein. In some embodiments, the antibody or ligand is displayed on the surface. Suitable techniques for bacterial surface display are described herein.

As GITR functions to promote T-cell proliferation and T-cell survival in activated T cells, GITR agonism may be advantageously combined with a second modality capable of initiating a T cell response (immune initiator), including but not limited to genetically engineered bacteria expressing a innate immune stimulator, such as a STING agonist, as described herein.

Accordingly, in one non-limiting example, one or more genetically engineered bacteria express one or more enzymes for the production of a STING agonist e.g., as described herein in combination with an agonistic anti-GITR antibody. In another non-limiting example, one or more genetically engineered bacteria express one or more enzymes for the production of a STING agonist e.g., as described herein are administered in combination with agonistic anti-GITR antibody, as described herein.

CD137 or 4-1BB is a type 2 transmembrane glycoprotein belonging to the TNF superfamily, which is expressed and has a co-stimulatory activity on activated T Lymphocytes (e.g., CD8+ and CD4+ cells). It has been shown to enhance T cell proliferation, IL-2 secretion survival and cytolytic activity. In some embodiments, the immune modulator is an agonist of CD137 (4-1BB), for example, an agonist selected from an agonistic anti-CD137 antibody or fragment thereof, or a CD137 ligand polypeptide or fragment thereof. Thus, in some embodiments, the genetically engineered bacteria comprise sequence(s) encoding an agonistic anti-CD137 antibody or fragment thereof, or a CD137 ligand polypeptide or fragment thereof. Thus, in some embodiments, the engineered bacteria is engineered to produce an agonistic anti-CD137 antibody or fragment thereof, or a CD137 ligand polypeptide or fragment thereof.

In some embodiments, the engineered bacteria comprises sequence to encode an agonistic anti-CD137 antibody or fragment thereof, or a CD137 ligand polypeptide or fragment thereof. In some embodiments, the genetically engineered bacterium expresses an agonistic anti-CD137 antibody or fragment thereof, or a CD137 ligand polypeptide or fragment thereof, and/or expresses secretory peptide(s). Non-limiting examples of suitable secretion tags and suitable secretory mechanisms are described herein. In some embodiments, the antibody or ligand is displayed on the surface. Suitable techniques for bacterial surface display are described herein.

CD137 (4-1BB) is expressed on activated mouse and human CD8+ and CD4+ T cells.7 It is a member of the TNFR family and mediates costimulatory and antiapoptotic functions, promoting T-cell proliferation and T-cell survival.10,11 CD137 has been reported to be up-regulated—depending on the T-cell stimulus—from 12 hours to up to 5 days after stimulation (Wolff et al., Activation-induced expression of CD137 permits detection, isolation, and expansion of the full repertoire of CD8 T cells responding to antigen without requiring knowledge of epitope specificities; BLOOD, 1 Jul. 2007 VOL. 110, NUMBER 1, and references therein). Accordingly CD137 (4-1BB) agonism may be advantageously combined with a second modality capable of initiating a T cell response (immune initiator), including but not limited to genetically engineered bacteria expressing a innate immune stimulator (immune initiator). Exemplary bacteria expressing a innate immune stimulator (immune initiator) are described herein.

Accordingly, in one non-limiting example, one or more genetically engineered bacteria express one or more enzymes for the production of a STING agonist e.g., as described herein in combination with an agonistic anti-41BB (CD137) antibody. In another non-limiting example, one or more genetically engineered bacteria express one or more enzymes for the production of a STING agonist e.g., as described herein are administered in combination with agonistic anti-41BB (CD137) antibody, as described herein.

OX40, or CD134, is a T-cell receptor involved in preserving the survival of T cells and subsequently increasing cytokine production. OX40 has a critical role in the maintenance of an immune response and a memory response due to its ability to enhance survival. It also plays a significant role in both Th1 and Th2 mediated reactions. In some embodiments, the immune modulator is an agonist of OX40, for example, an agonist selected from an agonistic anti-OX40 antibody or fragment thereof, or an OX40 ligand (OX40L) or fragment thereof. Thus, in some embodiments, the genetically engineered bacteria comprise sequence(s) encoding an agonistic anti-OX40 antibody or fragment thereof, or an OX40 ligand or fragment thereof. Thus, in some embodiments, the engineered bacteria is engineered to produce an agonistic anti-OX40 antibody or fragment thereof, or an OX40 ligand or fragment thereof. In some embodiments, the engineered bacteria comprises sequence to encode an agonistic anti-OX40 antibody or fragment thereof, or an OX40 ligand or fragment thereof. In some embodiments, the engineered bacteria comprises sequence(s) to encode an agonistic anti-OX40 antibody or fragment thereof, or an OX40 ligand or fragment thereof and sequence to encode a secretory peptide(s) for the secretion of said antibodies and polypeptides. Non-limiting examples of suitable secretion tags and suitable secretory mechanisms are described herein. In some embodiments, the antibody or ligand is displayed on the surface. Suitable techniques for bacterial surface display are described herein.

Recently, the combination of unmethylated CG-enriched oligodeoxynucleotide (CpG)-a Toll-like receptor 9 (TLR9) ligand- and anti-OX40 antibody injected locally into one site of a tumor was found to synergistically trigger a T cell immune response locally that then attacks cancer throughout the body at distal sites (Sagiv-Barfi et al., Eradication of spontaneous malignancy by local immunotherapy; Sci. Transl. Med. 10, eaan4488 (2018)). Unmethylated CG-enriched oligodeoxynucleotides (CpG) activate TLR9, a component of the innate immune system. Accordingly other mechanisms of activation the immune system may produce similar results in combination with an agonistic OX40 antibody, including but not limited to genetically engineered bacteria expressing a innate immune stimulator (immune initiator). Exemplary bacteria expressing a innate immune stimulator (immune initiator) are described herein.

Accordingly, in one non-limiting example, one or more genetically engineered bacteria express one or more enzymes for the production of a STING agonist e.g., as described herein in combination with an agonistic OX40 antibody. In another non-limiting example, one or more genetically engineered bacteria express one or more enzymes for the production of a STING agonist e.g., as described herein are administered in combination with an OX40 antibody, as described herein.

CD28 is one of the proteins expressed on T cells that provide co-stimulatory signals required for T cell activation and survival. In some embodiments, the immune modulator is an agonist of CD28, for example, an agonist selected from agonistic anti-CD28 antibody, agonistic anti-CD28 antibody fragment, CD80 (B7.1) polypeptide or polypeptide fragment thereof, and CD86 (B7.2) polypeptide or polypeptide fragment thereof. Thus, in some embodiments, the genetically engineered bacteria comprise sequence(s) encoding an agonistic anti-CD28 antibody or a fragment thereof, or a CD80 polypeptide or a fragment thereof, or a CD86 polypeptide or a fragment thereof. In some embodiments, the engineered bacteria is engineered to produce an agonistic anti-CD28 antibody or a fragment thereof, or a CD80 polypeptide or a fragment thereof, or a CD86 polypeptide or a fragment thereof. In some embodiments, the engineered bacteria comprises sequence to encode an agonistic anti-CD28 antibody or a fragment thereof, or a CD80 polypeptide or a fragment thereof, or a CD86 polypeptide or a fragment thereof. In some embodiments, the engineered bacteria comprises sequence(s) to encode an agonistic anti-CD28 antibody or a fragment thereof, or a CD80 polypeptide or a fragment thereof, or a CD86 polypeptide or a fragment thereof and sequence to encode a secretory peptide(s) for the secretion of said antibodies and polypeptides. Non-limiting examples of suitable secretion tags and suitable secretory mechanisms are described herein. In some embodiments, the antibody or ligand is displayed on the surface. Suitable techniques for bacterial surface display are described herein.

ICOS is an inducible T-cell co-stimulator structurally and functionally related to CD28. In some embodiments, the immune modulator is an agonist of ICOS, for example, an agonist selected from an agonistic anti-ICOS antibody or fragment thereof, or ICOS ligand polypeptide or fragment thereof. Thus, in some embodiments, the genetically engineered bacteria comprise sequence(s) encoding an agonistic anti-ICOS antibody or fragment thereof, or ICOS ligand polypeptide or fragment thereof. Thus, in some embodiments, the engineered bacteria is engineered to produce an agonistic anti-ICOS antibody or fragment thereof, or ICOS ligand polypeptide or fragment thereof. In some embodiments, the engineered bacteria comprises sequence to encode an agonistic anti-ICOS antibody or fragment thereof, or ICOS ligand polypeptide or fragment thereof. In some embodiments, the engineered bacteria comprises sequence(s) to encode an agonistic anti-ICOS antibody or fragment thereof, or ICOS ligand polypeptide or fragment thereof and sequence to encode a secretory peptide(s) for the secretion of said antibodies and polypeptides. Non-limiting examples of suitable secretion tags and suitable secretory mechanisms are described herein. In some embodiments, the antibody or ligand is displayed on the surface. Suitable techniques for bacterial surface display are described herein.

CD226 is a glycoprotein expressed on the surface of natural killer cells, platelets, monocytes, and a subset of T cells (e.g., CD8+ and CD4+ cells), which mediates cellular adhesion to other cells bearing its ligands, CD112 and CD155. Among other things, it is involved in immune synapse formation and triggers Natural Killer (NK) cell activation. In some embodiments, the immune modulator is an agonist of CD226, for example, an agonist selected from agonistic anti-CD226 antibody or fragment thereof, CD112 or CD155 polypeptide or fragments thereof. Thus, in some embodiments, the genetically engineered bacteria comprise sequence(s) encoding an agonist selected from agonistic anti-CD226 antibody or fragment thereof, CD112 or CD155 polypeptide or fragments thereof. Thus, in some embodiments, the engineered bacteria is engineered to produce an agonist selected from agonistic anti-CD226 antibody or fragment thereof, CD112 or CD155 polypeptide or fragments thereof. In some embodiments, the engineered bacteria comprises sequence to encode an agonist selected from agonistic anti-CD226 antibody or fragment thereof, CD112 or CD155 polypeptide or fragments thereof. In some embodiments, the engineered bacteria comprises sequence(s) to encode an agonist selected from agonistic anti-CD226 antibody or fragment thereof, CD112 or CD155 polypeptide or fragments thereof and sequence to encode a secretory peptide(s) for the secretion of said antibodies and polypeptides. Non-limiting examples of suitable secretion tags and suitable secretory mechanisms are described herein. In some embodiments, the antibody or ligand is displayed on the surface. Suitable techniques for bacterial surface display are described herein.

In any of these embodiments, the agonistic antibody may be a human antibody or humanized antibody and may comprise different isotypes, e.g., human IgG1, IgG2, IgG3 and IgG4's. Also, the antibody may comprise a constant region that is modified to increase or decrease an effector function such as FcR binding, FcRn binding, complement function, glycosylation, C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR). In any of these embodiments, the antibody may be a single chain antibody or a single chain antibody fragment.

In some embodiments, the genetically engineered microorganisms are capable of expressing any one or more of the described agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides are controlled by a promoter inducible by such conditions and/or inducers. In some embodiments, the gene sequences(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described genes sequences encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides are present on one or more plasmids (e.g., high copy or low copy) or are integrated into one or more sites in the microorganismal chromosome.

In any of these embodiments, the genetically engineered bacteria comprising gene sequence(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides further comprise gene sequence(s) encoding one or more further effector molecule(s), i.e., therapeutic molecule(s) or a metabolic converter(s). In any of these embodiments, the circuit encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described circuits encoding agonistic anti-GITR antibody/GITR ligand, anti-CD137/CD137 ligand, anti-OX40 antibody/OX40 ligand, anti-CD28 antibody/CD80 or CD86 polypeptides, anti-ICOS antibody/ICOS ligand, anti-CD226 antibody/CD112 and/or CD155 polypeptides and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Elimination (Reversal) of Local Immune Suppression

Tumor cells often escape destruction by producing signals that interfere with antigen presentation or maturation of dendritic cells, causing their precursors to mature into immunosuppressive cell types instead. Therefore, the local delivery of one or more immune modulators that prevent or inhibit the activities of immunomodulatory molecules involved in initiating, promoting and/or maintaining immunosuppression at the tumor site, alone or in combination with one or more other immune modulators, provides a therapeutic benefit.

### Immune Checkpoint Inhibitors

In some embodiments, the immune modulator is an inhibitor of an immune suppressor molecule, for example, an inhibitor of an immune checkpoint molecule. The immune checkpoint molecule to be inhibited can be any known or later discovered immune checkpoint molecule or other immune suppressor molecule. In some embodiments, the immune checkpoint molecule, or other immune suppressor molecule, to be inhibited is selected from CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR. In certain aspects, the present disclosure provides an engineered microorganism, e.g., engineered bacteria, that is engineered to produce one or more immune modulators that inhibit an immune checkpoint or other immune suppressor molecule. In some embodiments, the genetically engineered microorganisms are capable of reducing cancerous cell proliferation, tumor growth, and/or tumor volume. In some embodiments, the genetically engineered bacterium is bacterium that has been engineered to target a cancer or tumor cell. In some embodiments, the genetically engineered microorganism is a bacterium that expresses an immune checkpoint inhibitor, or inhibitor of another immune suppressor molecule, under the control of a promoter that is activated by low-oxygen conditions, e.g., the low-oxygen environment of a tumor. In some embodiments, the genetically engineered bacterium express one or more immune checkpoint inhibitors, under the control of a promoter that is activated by hypoxic conditions or by inflammatory conditions, such as any of the promoters activated by said conditions and described herein.

In some embodiments, the genetically engineered microorganisms of the disclosure are genetically engineered bacteria comprising a gene encoding a CTLA-4 inhibitor, for example, an antibody directed against CTLA-4. In any of these embodiments, the anti-CTLA-4 antibody may be a single-chain anti-CTLA-4 antibody. In some embodiments, the genetically engineered microorganisms of the disclosure are genetically engineered bacteria comprising a gene encoding a PD-1 inhibitor, for example, an antibody directed against PD-1 or PD-L1. In any of these embodiments, the anti-PD-1 or PD-L1 antibody may be a single-chain anti-PD-1 antibody. In some embodiments, the genetically engineered microorganisms of the disclosure are engineered bacteria comprising a gene encoding an inhibitor selected from CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR inhibitors, e.g., an antibody directed against any of the listed immune checkpoints or other suppressor molecules. Examples of such checkpoint inhibitor molecules are described e.g., in International Patent Application PCT/US2017/013072, filed Jan. 11, 2017, published as WO2017/123675, and PCT/US2018/012698, filed Jan. 1, 2018, the contents of each of which is herein incorporated by reference in its entirety. In any of these embodiments, the antibody may be a single-chain antibody. In some embodiments, the engineered bacteria expressing a checkpoint inhibitor, or inhibitor of another immune suppressor molecule, is administered locally, e.g., via intratumoral injection.

In some embodiments, the disclosure provides a genetically engineered microorganism, e.g., engineered bacterium, that expresses a CTLA-4 inhibitor. In some embodiments, the genetically engineered bacterium expresses a CTLA-4 inhibitor under the control of a promoter that is activated by low-oxygen conditions, e.g., the hypoxic environment of a tumor. In some embodiments, the genetically engineered bacterium expresses an anti-CTLA-4 antibody, for example, a single chain antibody. In some embodiments, the genetically engineered bacterium is bacterium that expresses an anti-CTLA-4 antibody, for example, a single chain antibody. In some embodiments, the genetically engineered bacterium expresses an anti-CTLA-4 antibody, for example, a single chain antibody, under the control of a promoter that is activated by low-oxygen conditions. In some embodiments, the genetically engineered bacterium is a bacterium that expresses an anti-CTLA-4 antibody, for example, a single chain antibody, under the control of a promoter that is activated by low-oxygen conditions.

In some embodiments, the genetically engineered microorganism is a bacterium that expresses a PD-1 inhibitor. In some embodiments, the genetically engineered bacterium expresses a PD-1 inhibitor under the control of a promoter that is activated by low-oxygen conditions, e.g., the hypoxic environment of a tumor. In some embodiments, the genetically engineered microorganism is a bacterium that expresses a PD-1 inhibitor under the control of a promoter that is activated by low-oxygen conditions, e.g., the hypoxic environment of a tumor. In some embodiments, the genetically engineered bacterium expresses an anti-PD-1 antibody, e.g., single chain antibody. In some embodiments, the genetically engineered bacterium is a bacterium that expresses an anti-PD-1 antibody, e.g., single chain antibody. In some embodiments, the genetically engineered bacterium expresses an anti-PD-1 antibody, e.g., single chain antibody, under the control of a promoter that is activated by low-oxygen conditions. In some embodiments, the genetically engineered bacterium is a bacterium that expresses an anti-PD-1 antibody, e.g., single chain antibody, under the control of a promoter that is activated by low-oxygen conditions.

In some embodiments, the nucleic acid encoding an scFv construct, e.g., a PD1-scFv, comprises a sequence which has at least about 80%, at least about 85%, at least about 90%, at least about 95%, or at least about 99% identity to a sequence selected from SEQ ID NO: 975, SEQ ID NO: 976, SEQ ID NO: 977, SEQ ID NO: 978, SEQ ID NO: 979, and/or SEQ ID NO: 980. In some embodiments, the nucleic acid encoding an scFv construct, e.g., a PD1-scFv, comprises a sequence selected from SEQ ID NO: 975, SEQ ID NO: 976, SEQ ID NO: 977, SEQ ID NO: 978, SEQ ID NO: 979, and/or SEQ ID NO: 980. In some embodiments, the nucleic acid encoding an scFv construct, e.g., a PD1-scFv, consists of a sequence selected from SEQ ID NO: 975, SEQ ID NO: 976, SEQ ID NO: 977, SEQ ID NO: 978, SEQ ID NO: 979, and/or SEQ ID NO: 980.

In some embodiments, the genetically engineered bacterium expresses a PD-L1 inhibitor. In some embodiments, the genetically engineered bacterium expresses an anti-PD-L1 antibody, e.g., single chain antibody. In some embodiments, the genetically engineered bacterium is a bacterium that expresses an anti-PD-L1 antibody, e.g., single chain antibody. In some embodiments, the genetically engineered bacterium is a bacterium that expresses an anti-PD-L1 antibody, e.g., single chain antibody under the control of a promoter that is activated by low-oxygen conditions.

In some embodiments, the genetically engineered bacterium is a bacterium that expresses an PD-L2 inhibitor. In some embodiments, the genetically engineered bacterium is a bacterium that expresses an anti-PD-L2 antibody, e.g., single chain antibody. In some embodiments, the genetically engineered bacterium is a bacterium that expresses an anti-PD-L2 antibody, e.g., single chain antibody. In some embodiments, the genetically engineered bacterium is a bacterium that expresses an anti-PD-L2 antibody, e.g., single chain antibody under the control of a promoter that is activated by low-oxygen conditions.

Exemplary heavy and light chain amino acid sequences for use in constructing single-chain anti-CTLA-4 antibodies are shown are described herein (e.g., SEQ ID NO: 761, SEQ ID NO: 762, SEQ ID NO: 763, SEQ ID NO: 764).

Exemplary heavy and light chain amino acid sequences for use in constructing single-chain anti-PD-1 antibodies include SEQ ID NO: 1, SEQ ID NO: 2, SEQ ID NO: 3, and/or SEQ ID NO: 4.

In some embodiments, the sequence is at least about 80%, at least about 85%, at least about 90%, at least about 95%, or at least about 99% homologous to the sequence of SEQ ID NO: 1, SEQ ID NO: 2, SEQ ID NO: 3, and/or SEQ ID NO: 4. Other exemplary heavy and light chain amino acid sequences for construction of single chain antibodies include SEQ ID NO: 5-46.

In some embodiments, the single chain antibody is at least about 80%, at least about 85%, at least about 90%, at least about 95%, or at least about 99% homologous to the sequence of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44 SEQ ID NO:45, or SEQ ID NO: 46.

In some embodiments, genetically engineered bacteria comprise a nucleic acid sequence that encodes a polypeptide that is at least about 80%, at least about 85%, at least about 90%, at least about 95%, or at least about 99% homologous to the DNA sequence of SEQ ID NO: 1, SEQ ID NO: 2, SEQ ID NO: 3, and/or SEQ ID NO: 4.

In some embodiments, the genetically engineered microorganisms are capable of expressing any one or more of the described CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR are controlled by a promoter inducible by such conditions and/or inducers. In some embodiments, the gene sequences(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described genes sequences encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR are present on one or more plasmids (e.g., high copy or low copy) or are integrated into one or more sites in the microorganismal chromosome.

In any of these embodiments, the genetically engineered bacteria comprising gene sequence(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR further comprise gene sequence(s) encoding one or more further effector molecule(s), i.e., therapeutic molecule(s) or a metabolic converter(s). In any of these embodiments, the circuit encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described circuits encoding CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, B7-H3, B7-H4, IDO, TDO, KIR, and A2aR and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Immuno-Metabolism and Metabolic Converters

### Tryptophan and Kynurenine

T regulatory cells, or Tregs, are a subpopulation of T cells that modulate the immune system by preventing excessive immune reactions, maintaining tolerance to self-antigens, and abrogating autoimmunity. Tregs suppress the immune responses of other cells, for example, shutting down immune responses after they have successfully eliminated invading organisms. These cells generally suppress or downregulate induction and proliferation of effector T cells. There are different sub-populations of regulatory T cells, including those that express CD4, CD25, and Foxp3 (CD4+CD25+ regulatory T cells). Tregs are key to dampening effector T cell responses, and therefore represent one of the main obstacles to effective anti-tumor response and the failure of current therapies that rely on induction or potentiation of anti-tumor responses. Thus, in certain embodiments, the genetically engineered bacteria of the present disclosure produce one or more immune modulators that deplete Tregs and/or inhibit or block the activation of Tregs.

The tryptophan (TRP) to kynurenine (KYN) metabolic pathway is established as a key regulator of innate and adaptive immunity. Both the degradation of the essential amino acid tryptophan via indoleamine-2,3-dioxygenase 1 (IDO1) and TRP-2,3-dioxygenase 2 (TDO) and the resulting production of aryl hydrocarbon receptor (AHR) activating tryptophan metabolites, such as kynurenine, is a central pathway maintaining the immunosuppressive microenvironment in many types of cancers. For example, binding of kynurenine to AHR results in reprograming the differentiation of naïve CD4+T-helper (Th) cells favoring a regulatory T cells phenotype (Treg) while suppressing the differentiation into interleukin-17 (IL-17)-producing Th (Th17) cells. Activation of the aryl hydrogen receptor also results in promoting a tolerogenic phenotype on dendritic cells.

In some embodiments, the genetically engineered microorganisms of the present disclosure, e.g., genetically engineered bacteria are capable of depleting Tregs or inhibiting or blocking the activation of Tregs by producing tryptophan and/or degrading kynurenine. In some embodiments, the genetically engineered microorganisms of the present disclosure capable of increasing the CD8+: Treg ratio (e.g., favors the production of CD8+ over Tregs) by producing tryptophan and/or degrading kynurenine.

### Increasing Tryptophan

In some embodiments, the genetically engineered microorganisms of the present disclosure are capable of producing tryptophan. In some embodiments, the genetically engineered bacteria and/or other microorganisms that produce tryptophan comprise one or more gene sequences encoding one or more enzymes of the tryptophan biosynthetic pathway. In some embodiments, the genetically engineered bacteria comprise sequence(s) encoding trpE, trpD, trpC, trpF, trpB, and trpA genes from B. subtilis or E. coli. and optionally comprise gene sequence(s) to produce the tryptophan precursor, chorismite, e.g., sequence(s) encoding aroG, aroF, aroH, aroB, aroD, aroE, aroK, and AroC, and optionally either a wild type or a feedback resistant SerA gene. Optionally, AroG and TrpE are replaced with feedback resistant versions. In any of these embodiments, the tryptophan repressor (trpR) optionally may be deleted, mutated, or modified so as to diminish or obliterate its repressor function. In any of these embodiments, the tnaA gene (encoding a tryptophanase converting Trp into indole) optionally may be deleted. Examples of such checkpoint inhibitor molecules are described e.g., in International Patent Application PCT/US2017/013072, filed Jan. 11, 2017, published as WO2017/123675, and PCT/US2018/012698, filed Jan. 1, 2018, the contents of each of which is herein incorporated by reference in its entirety.

In some embodiments, the genetically engineered bacteria are capable of expressing any one or more of the described circuits in low-oxygen conditions, and/or in the presence of cancer and/or the tumor microenvironment and/or the tumor microenvironment or tissue specific molecules or metabolites, and/or in the presence of molecules or metabolites associated with inflammation or immune suppression, and/or in the presence of metabolites that may be present in the tumor or the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during bacterial expansion, production and/or manufacture, as described herein.

In some embodiments, any one or more of the described circuits are present on one or more plasmids (e.g., high copy or low copy) or are integrated into one or more sites in the bacterial chromosome. Also, in some embodiments, the genetically engineered bacteria and/or other microorganisms are further capable of expressing any one or more of the described circuits and further comprise one or more of the following: (1) one or more initiator circuits, including but not limited to, one or more enzymes for the production of a STING agonist, as described herein, (2) one or more sustainer circuits, as described herein, (3) one or more auxotrophies, such as any auxotrophies known in the art and provided herein, e.g., thyA auxotrophy, (2) one or more kill switch circuits, described herein or otherwise known in the art, (3) one or more antibiotic resistance circuits, (4) one or more transporters for importing biological molecules or substrates, described herein or otherwise known in the art, (5) one or more secretion circuits, described herein and otherwise known in the art, (6) one or more surface display circuits, described herein and otherwise known in the art and (7) one or more circuits for the production or degradation of one or more metabolites (metabolic converters) (e.g., kynurenine, tryptophan, adenosine, arginine) described herein and (8) combinations of one or more of such additional circuits. In any of these embodiments, the genetically engineered bacteria may be administered alone or in combination with one or more immune checkpoint inhibitors described herein, including but not limited to anti-CTLA4 antibodies, anti-PD1 and/or anti-PDL1 antibodies.

### Decreasing Kynurenine

In some embodiments, the genetically engineered bacteria and/or other microorganisms comprise a mechanism for metabolizing or degrading kynurenine, and reducing kynurenine levels in the extracellular environment. In some embodiments, the genetically engineered bacteria and/or other microorganisms comprise gene sequence(s) encoding kynureninase.

In one embodiments, the genetically engineered microorganisms encode gene sequences for the expression of kynureninase from Pseudomonas fluorescens, which converts kynurenine to AA (Anthranillic acid), which then can be converted to tryptophan through the enzymes of the E. coli trp operon. Optionally, the trpE gene may be deleted as it is not needed for the generation of tryptophan from kynurenine. Accordingly, in one embodiment, the genetically engineered bacteria may comprise one or more gene(s) or gene cassette(s) encoding trpD, trpC, trpA, and trpD and kynureninase. This deletion may prevent tryptophan production through the endogenous chorismate pathway, and may increase the production of tryptophan from kynurenine through kynureninase.

In alternate embodiments, the trpE gene is not deleted, in order to maximize tryptophan production by using both kynurenine and chorismate as a substrate. In one embodiment of the invention, the genetically engineered bacteria and/or other microorganisms comprising this circuit may be useful for reducing immune escape in cancer.

In some embodiments, the microorganisms encode a transporter for the uptake of kynurenine from the extracellular environment, e.g., the tumor environment. AroT, located between chr and the trp operon in Salmonella typhimurium, and similar genes, aroR and aroS, near the trp locus of Escherichia coli, were found to be involved in the transport of aromatic amino acids. AroP is a permease that is involved in the transport across the cytoplasmic membrane of the aromatic amino acids (phenylalanine, tyrosine, and tryptophan). Expression of such transporters/permeases may be useful for kynurenine import in the genetically engineered microorganisms.

Exemplary genes encoding kynureninase which are encoded by the genetically engineered bacteria of the disclosure in certain embodiments include SEQ ID NO: 65-67

In one embodiment, one or more polypeptides and/or polynucleotides encoded and expressed by the genetically engineered bacteria have at least about 80% identity with one or more of SEQ ID NO: 65 through SEQ ID NO: 67. In one embodiment, one or more polypeptides and/or polynucleotides encoded and expressed by the genetically engineered bacteria have at least about 85% identity with one or more of SEQ ID NO: 65 through SEQ ID NO: 67. In one embodiment, one or more polypeptides and/or polynucleotides encoded and expressed by the genetically engineered bacteria have at least about 90% identity with one or more of SEQ ID NO: 65 through SEQ ID NO: 67. In one embodiment, one or more polypeptides and/or polynucleotides encoded and expressed by the genetically engineered bacteria have at least about 95% identity with one or more of SEQ ID NO: 65 through SEQ ID NO: 67. In one embodiment, one or more polypeptides and/or polynucleotides encoded and expressed by the genetically engineered bacteria have at least about 96%, 97%, 98%, or 99% identity with one or more of SEQ ID NO: 65 through SEQ ID NO: 67. Accordingly, in one embodiment, one or more polypeptides and/or polynucleotides expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 65 through SEQ ID NO: 67. In another embodiment, one or more polynucleotides and/or polypeptides encoded and expressed by the genetically engineered bacteria comprise the sequence of one or more of SEQ ID NO: 65 through SEQ ID NO: 67. In another embodiment, one or more polynucleotides and/or polypeptides encoded and expressed by the genetically engineered bacteria consist of the sequence of one or more of SEQ ID NO: 65 through SEQ ID NO: 67.

Exemplary codon-optimized kynureninase cassette sequences include SEQ ID NO: 68, 865, 69, 866, 70, 867. In one embodiment, one or more polynucleotides encoded and expressed by the genetically engineered bacteria have at least about 80% identity with one or more of SEQ ID NO: 68 through SEQ ID NO: 70 and SEQ ID NO: 865 through SEQ ID NO: 868. In one embodiment, one or more polynucleotides encoded and expressed by the genetically engineered bacteria have at least about 85% identity with one or more of SEQ ID NO: 68 through SEQ ID NO: 70 and SEQ ID NO: 865 through SEQ ID NO: 868. In one embodiment, one or more polynucleotides encoded and expressed by the genetically engineered bacteria have at least about 90% identity with one or more of SEQ ID NO: 68 through SEQ ID NO: 70 and SEQ ID NO: 865 through SEQ ID NO: 868. In one embodiment, one or more polynucleotides encoded and expressed by the genetically engineered bacteria have at least about 95% identity with one or more of SEQ ID NO: 68 through SEQ ID NO: 70 and SEQ ID NO: 865 through SEQ ID NO: 868. In one embodiment, one or more polynucleotides encoded and expressed by the genetically engineered bacteria have at least about 96%, 97%, 98%, or 99% identity with one or more of SEQ ID NO: 68 through SEQ ID NO: 70 and SEQ ID NO: 865 through SEQ ID NO: 868. Accordingly, in one embodiment, one or more polynucleotides expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 68 through SEQ ID NO: 70 and SEQ ID NO: 865 through SEQ ID NO: 868. In another embodiment, one or more polynucleotides encoded and expressed by the genetically engineered bacteria comprise the sequence of one or more of SEQ ID NO: 68 through SEQ ID NO: 70 and SEQ ID NO: 865 through SEQ ID NO: 868. In another embodiment, one or more polynucleotides encoded and expressed by the genetically engineered bacteria consists of the sequence of one or more of SEQ ID NO: 68 through SEQ ID NO: 70 and SEQ ID NO: 865 through SEQ ID NO: 868.

In some embodiments, the construct for expression of Pseudomonas fluorescens Kynureninase is at least about 80%, at least about 85%, at least about 90%, at least about 95%, or at least about 99% homologous to a sequence selected from SEQ ID NO: 116, SEQ ID NO: 888, SEQ ID NO: 889, SEQ ID NO: 890, SEQ ID NO: 891, SEQ ID NO: 892, and/or SEQ ID NO: 893. In some embodiments, the construct for expression of Pseudomonas fluorescens Kynureninase comprises a sequence selected from SEQ ID NO: 116, SEQ ID NO: 888, SEQ ID NO: 889, SEQ ID NO: 890, SEQ ID NO: 891, SEQ ID NO: 892, and/or SEQ ID NO: 893. In some embodiments, the construct for expression of Pseudomonas fluorescens Kynureninase consists of a sequence selected from SEQ ID NO: 116, SEQ ID NO: 888, SEQ ID NO: 889, SEQ ID NO: 890, SEQ ID NO: 891, SEQ ID NO: 892, and/or SEQ ID NO: 893. Other suitable kynureninase are described in US Patent Publication 20170056449, the contents of which is herein incorporated by reference in its entirety.

In any of these embodiments, the bacteria genetically engineered to consume kynurenine and optionally produce tryptophan consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more kynurenine than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more kynurenine than unmodified bacteria of the same bacterial subtype under the same conditions. In yet another embodiment, the genetically engineered bacteria consume about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, one-thousand-fold, or more greater amounts of kynurenine than unmodified bacteria of the same bacterial subtype under the same conditions.

In any of these embodiments, the bacteria genetically engineered to consume kynurenine and optionally produce tryptophan produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more tryptophan than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more tryptophan than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more tryptophan than unmodified bacteria of the same bacterial subtype under the same conditions.

In any of these embodiments, the genetically engineered bacteria increase the kynurenine consumption rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the kynurenine consumption rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase the kynurenine consumption rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one embodiment, the genetically engineered bacteria increase the kynurenine consumption by about 80% to 100% relative to unmodified bacteria of the same bacterial subtype under the same conditions, after 4 hours. In one embodiment, the genetically engineered bacteria increase the kynurenine consumption by about 90% to 100% relative to unmodified bacteria of the same bacterial subtype under the same conditions after 4 hours. In one specific embodiment, the genetically engineered bacteria increase the kynurenine consumption by about 95% to 100% relative to unmodified bacteria of the same bacterial subtype under the same conditions, after 4 hours. In one specific embodiment, the genetically engineered bacteria increase the kynurenine consumption by about 99% to 100% relative to unmodified bacteria of the same bacterial subtype under the same conditions, after 4 hours. In yet another embodiment, the genetically engineered bacteria increase the kynurenine consumption by about 10-50 fold after 4 hours. In yet another embodiment, the genetically engineered bacteria increase the kynurenine consumption by about 50-100 fold after 4 hours. In yet another embodiment, the genetically engineered bacteria increase the kynurenine consumption by about 100-500 fold after 4 hours. In yet another embodiment, the genetically engineered bacteria increase the kynurenine consumption by about 500-1000 fold after 4 hours. In yet another embodiment, the genetically engineered bacteria increase the kynurenine consumption by about 1000-5000 fold after 4 hours. In yet another embodiment, the genetically engineered bacteria increase the kynurenine consumption by about 5000-10000 fold after 4 hours. In yet another embodiment, the genetically engineered bacteria increase the kynurenine consumption by about 10000-1000 fold after 4 hours.

In any of these embodiments, the genetically engineered bacteria are capable of reducing cell proliferation, e.g., in the tumor, by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the kynureninase is secreted into the extracellular environment, e.g., tumor microenvironment, using a secretion system described herein.

In some embodiments, the genetically engineered bacteria and/or other microorganisms comprise a mechanism for metabolizing or degrading kynurenine, which, in some embodiments, also results in the increased production of tryptophan. In some embodiments, the genetically engineered bacteria modulate the TRP: KYN ratio or the KYN: TRP ratio in the extracellular environment. In some embodiments, the genetically engineered bacteria increase the TRP: KYN ratio or the KYN: TRP ratio. In some embodiments, the genetically engineered bacteria reduce the TRP: KYN ratio or the KYN: TRP ratio. In some embodiments, the genetically engineered bacteria comprise sequence encoding the enzyme kynureninase, and further any of the tryptophan production circuits described herein.

The genetically engineered bacteria and/or other microorganisms may comprise any suitable gene for producing kynureninase. In some embodiments, the gene for producing kynureninase is modified and/or mutated, e.g., to enhance stability, increase kynureninase production. In some embodiments, the engineered bacteria and/or other microorganisms also have enhanced uptake or import of kynurenine, e.g., comprise a transporter or other mechanism for increasing the uptake of kynurenine into the bacteria and/or other microorganisms cell.

In some embodiments, the genetically engineered bacteria and/or other microorganisms are capable of producing kynureninase under inducing conditions, e.g., under a condition(s) associated with immune suppression and/or tumor microenvironment. In some embodiments, the genetically engineered bacteria and/or other microorganisms are capable of producing kynureninase in low-oxygen conditions, in the presence of certain molecules or metabolites associated with cancer, or certain tissues, immune suppression, or inflammation, or in the presence of some other metabolite that may or may not be present in vivo, e.g, in the tumor microenvironment, and may be present in vitro during strain culture, expansion, production and/or manufacture, such as arabinose, cumate, and salicylate.

In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during bacteria and/or other microorganismal expansion, production and/or manufacture, as described herein. In some embodiments, any one or more of the described circuits are present on one or more plasmids (e.g., high copy or low copy) or are integrated into one or more sites in the bacterial chromosome.

In any of these embodiments, the genetically engineered bacteria comprising gene sequence(s) encoding kynureninase, e.g., from Pseudomonas fluorescens, further comprise gene sequence(s) encoding one or more further effector molecule(s), i.e., therapeutic molecule(s) or a metabolic converter(s). In any of these embodiments, the circuit encoding kynureninase, e.g., from Pseudomonas fluorescens, may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding kynureninase, e.g., from Pseudomonas fluorescens, may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding kynureninase, e.g., from Pseudomonas fluorescens, encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding kynureninase, e.g., from Pseudomonas fluorescens, encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein. Optionally the bacterial strain may further comprise tryptophan production circuitry described herein.

Also, in some embodiments, the genetically engineered bacteria and/or other microorganisms are further capable of expressing any one or more of the described circuits and further comprise one or more of the following: (1) one or more initiator circuits, including but not limited to, one or more enzymes for the production of a STING agonist, as described herein, (2) one or more sustainer circuits, as described herein, (3) one or more auxotrophies, such as any auxotrophies known in the art and provided herein, e.g., thyA auxotrophy, (2) one or more kill switch circuits, described herein or otherwise known in the art, (3) one or more antibiotic resistance circuits, (4) one or more transporters for importing biological molecules or substrates, described herein or otherwise known in the art, (5) one or more secretion circuits, described herein and otherwise known in the art, (6) one or more surface display circuits, described herein and otherwise known in the art and (7) one or more circuits for the production or degradation of one or more metabolites (metabolic converters) (e.g., kynurenine, tryptophan, adenosine, arginine) described herein and (8) combinations of one or more of such additional circuits. In any of these embodiments, the genetically engineered bacteria may be administered alone or in combination with one or more immune checkpoint inhibitors described herein, including but not limited to anti-CTLA4 antibodies, anti-PD1 and/or anti-PDL1 antibodies.

### Adaptive Laboratory Evolution (ALE)

E. coli Nissle can be engineered to efficiently import KYN and convert it to TRP by introducing the Kynureninase (KYNase) from Pseudomonas fluorescens (kynU) as described herein.

E. coli naturally utilizes anthranilate in its TRP biosynthetic pathway. Briefly, the TrpE (in complex with TrpD) enzyme converts chorismate into anthranilate. TrpD, TrpC, TrpA and TrpB then catalyze a five-step reaction ending with the condensation of an indole with serine to form tryptophan. By replacing the TrpE enzyme via lambda-RED recombineering, the subsequent strain of Nissle (AtrpE::Cm) is an auxotroph unable to grow in minimal media without supplementation of TRP or anthranilate. By expressing kynureninase in ΔtrpE::Cm (KYNase-trpE), this auxotrophy can be alternatively rescued by providing KYN.

As described herein, leveraging the growth-limiting nature of KYN in KYNase-trpE, adaptive laboratory evolution was employed to evolve a strain capable of increasingly efficient utilization of KYN.

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Adaptive laboratory evolution is described in International Patent Application PCT/US2017/013072, filed Jan. 11, 2017, published as WO2017/123675, the contents of which is herein incorporated by reference in its entirety.

First a lower limit of KYN concentration was established and mutants were evolved by passaging in lowering concentrations of KYN. While this can select for mutants capable of increasing KYN import, the bacterial cells still prefer to utilize free, exogenous TRP. In the tumor environment, dual-therapeutic functions can be provided by depletion of KYN and increasing local concentrations of TRP. Therefore, to evolve a strain which prefers KYN over TRP, a toxic analogue of TRP—5-fluoro-L-tryptophan (ToxTRP)—can be incorporated into the ALE experiment. The resulting best performing strain is then whole genome sequenced in order to deconvolute the contributing mutations. Lambda-RED can be performed in order to reintroduce TrpE, to inactivate Trp regulation (trpR, tyrR, transcriptional attenuators) to up-regulate TrpABCDE expression and increase chorismate production. The resulting strain is now insensitive to external TRP, efficiently converts KYN into TRP, and also now overproduces TRP.

### Purinergic System—ATP/Adenosine Metabolism

An important barrier to successful cancer immunotherapy is that tumors employ a number of mechanisms to facilitate immune escape, including the production of anti-inflammatory cytokines, the recruitment of regulatory immune subsets, and the production of immunosuppressive metabolites. One such immunosuppressive pathway is the production of extracellular adenosine, a potent immunosuppressive molecule, by CD73 Immune-stimulatory extracellular ATP, released by damaged or dying cells and bacteria, promotes the recruitment of immune phagocytes and activates P2×7R, a coactivator of the NLRP3 inflammasome, which then triggers the production of proinflammatory cytokines, such as IL-1β and IL-18. The catabolism of extracellular ATP into ADP, AMP and adenosine is controlled by CD39 (ecto-nucleoside triphosphate diphosphohydrolase 1, E-NTPDase1) which hydrolyzes ATP into AMP, and CD73 (ecto-5′-nucleotidase, Ecto5′NTase) which dephosphorylates AMP into adenosine by. Thus, CD39 and CD73 act in concert to convert proinflammatory ATP into immunosuppressive adenosine. Beside its immunoregulatory roles, the ectonucleotidase pathway contributes directly to the modulation of cancer cell growth, differentiation, invasion, migration, metastasis, and tumor angiogenesis.

In some embodiments, the genetically engineered bacteria comprise a means for removing excess adenosine from the tumor microenvironment. Many bacteria scavenge low concentrations of nucleosides from the environment for synthesis of nucleotides and deoxynucleotides by salvage pathways of synthesis. Additionally, in Escherichia coli, nucleosides can be used as the sole source of nitrogen and carbon for growth (Neuhard J, Nygaard P. Biosynthesis and conversion of nucleotides, purines and pyrimidines. In: Neidhardt F C, Ingraham J L, Low K B, Magasanik B, Schaechter M, Umbarger H E, editors. Escherichia coli and Salmonella typhimurium: Cellular and molecular biology. Washington D.C.: ASM Press; 1987. pp. 445-473). Two evolutionarily unrelated cation-linked transporter families, the Concentrative Nucleoside Transporter (CNT) family and the Nucleoside: H+ Symporter (NHS) family, are responsible for nucleoside uptake (see e.g., Cabrita et al., Biochem. Cell Biol. Vol. 80, 2002. Molecular biology and regulation of nucleoside and nucleobase transporter proteins in eukaryotes and prokaryotes), the contents of which is herein incorporated by reference in its entirety. NupC and NupG, are the transporter family members in E. coli. Mutants defective in both the nupC and nupG genes cannot grow with nucleosides as a single carbon source. Both of these transporters are proton-linked but they differ in their selectivity. NupC is a nucleotide transporter of the H+/nucleotide symporter family. NupC pyrimidine nucleoside-H+ transporter mediates symport (i.e., H+-coupled substrate uptake) of nucleosides, particularly pyrimidines. Two known members of the family are found in gram positive and gram-negative bacteria. NupG is capable of transporting a wide range of nucleosides and deoxynucleosides; in contrast, NupC does not transport guanosine or deoxyguanosine. Homologs of NupG from E. coli are found in a wide range of eubacteria, including human gut pathogens such as Salmonella typhimurium, organisms associated with periodontal disease such as Porphyromonas gingivalis and Prevotella intermedia, and plant pathogens in the genus Erwinia (As described in Vaziri et al., Mol Membr Biol. 2013 March; 30(1-2): 114-128; Use of molecular modelling to probe the mechanism of the nucleoside transporter NupG, the contents of which is herein incorporated by reference in its entirety). Putative bacterial transporters from the CNT superfamily and transporters from the NupG/XapB family include those listed in the Table 5 and Table 6 below. In addition, codB (GenBank P25525, Escherichia coli) was identified based on homology to a yeast transporter family termed the uracil/allantoin transporter family (Cabrita et al., supra).

TABLE 5
Putative CNT family transporters

| Name | GenBank Acc. No. | Organism |
|---|---|---|
| BH1446 | BAB05165 | Bacillus halodurans |
| BsNupC | CAA57663 | B. subtilis |
| BsyutK | CAB15208 | B. subtilis |
| BsyxjA | CAB15938 | B. subtilis |
| CcCNT (CC2089) | AAK24060 | Caulobacter crescentus |
| (yeiJ) | AAC75222 | E. coli |
| (yeiM) | AAC75225 | E. coil |
| (HI0519) | AAC22177 | Haemophilus influenzae |
| (HP1180) | AAD08224 | Helicobacter pylori |
| (SA0600, SAV0645) | BAB41833, BAB56807 | Staphylococcus aureus |
| SpNupC | AAK34582 | Streptococcus pyogenes |
| (VC2352) | AAF95495 | Vibrio cholerae |
| (VC1953) | AAF95101 | V. cholera |
| (VCA0179) | AAF96092 | V. cholera |

TABLE 6
Bacterial transporters from the NupG/XapB family

| Protein (gene name) | GenBank accession No. | Organism |
|---|---|---|
| 1. yegT | P76417 | Escherichia coli |
| 2. NupG | P09452 | E. coli |
| 3. XapB | P45562 | E. coli |
| 4. (CC1628) | AAK23606 | Caulobacter crescentus |

In some embodiments, the genetically engineered bacteria comprise a means for importing adenosine into the engineered bacteria from the tumor microenvironment. In some embodiments, the genetically engineered bacteria comprise sequence for encoding a nucleoside transporter. In some embodiments, the genetically engineered bacteria comprise sequence for encoding an adenosine transporter. In certain embodiments, genetically engineered bacteria comprise sequence for encoding E. coli Nucleoside Permease nupG or nupC. In any of these embodiments, the genetically engineered bacterium is bacterium for intratumoral administration. In some embodiments, the genetically engineered bacterium comprises sequence for encoding a nucleoside transporter or an adenosine transporter, e.g., nupG or nupC transporter sequence, under the control of a promoter that is activated by low-oxygen conditions. In some embodiments, the genetically engineered bacterium comprises sequence for encoding a nucleoside transporter or an adenosine transporter, e.g., nupG or nupC transporter sequence, under the control of a promoter that is activated by hypoxic conditions, or by inflammatory conditions, such as any of the promoters activated by said conditions and described herein. In some embodiments, the genetically engineered bacteria comprises sequence for encoding a nucleoside transporter or an adenosine transporter, e.g., nupG or nupC transporter sequence, under the control of a cancer-specific promoter, a tissue-specific promoter, or a constitutive promoter, such as any of the promoters described herein.

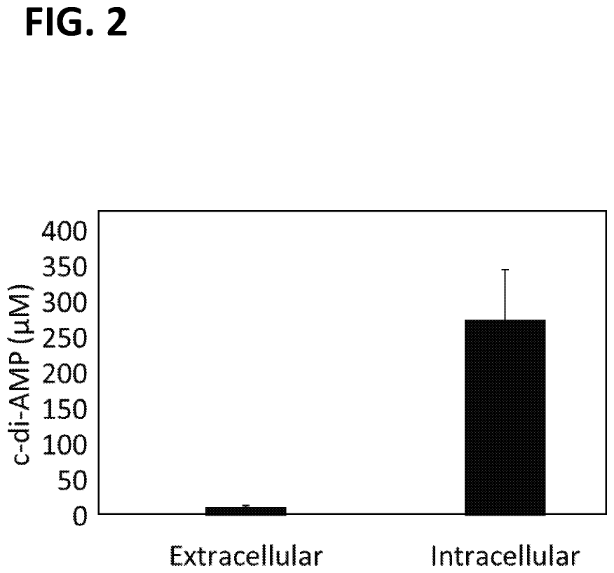

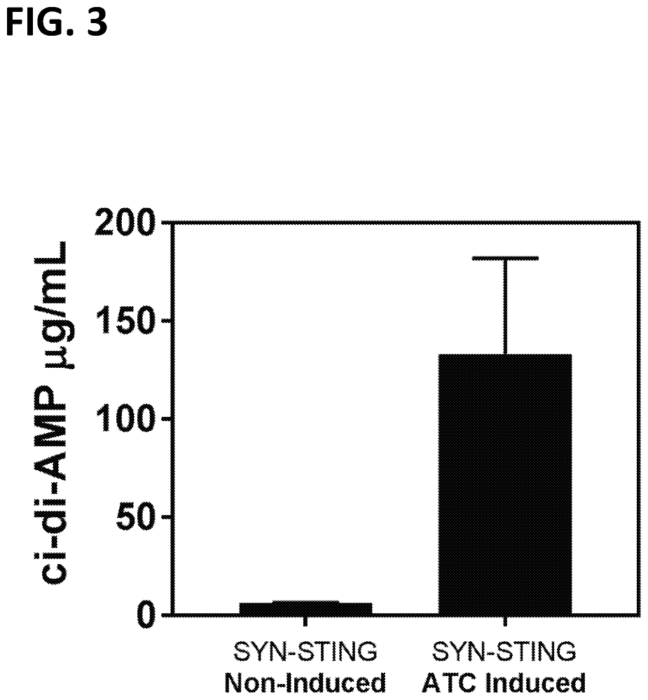

In some embodiments, the genetically engineered bacteria comprise a means for metabolizing or degrading adenosine. In some embodiments, the genetically engineered bacteria comprise one or more gene sequences encoding one or more enzymes that are capable of converting adenosine to urate (See FIG. 1, FIG. 2, and FIG. 3). In some embodiments, the genetically engineered bacteria comprise sequence(s) encoding add, xapA, deoD, xdhA, xdhB, and xdhC genes from E. coli. In some embodiments, the genetically engineered bacteria comprise sequence(s) encoding add, xapA, deoD, xdhA, xdhB, and xdhC genes from E. coli and comprise sequence encoding a nucleoside or adenosine transporter. In some embodiments, the genetically engineered bacteria comprise sequence(s) encoding add, xapA, deoD, xdhA, xdhB, and xdhC genes from E. coli and comprise sequence encoding nupG or nupC. An exemplary engineered bacteria is shown in FIG. 2.

Exemplary sequences useful for adenosine degradation circuits include SEQ ID NO: 71-77.

In some embodiments, genetically engineered bacteria comprise a nucleic acid sequence encoding an adenosine degradation enzyme or adenosine transporter that has at least about 80% identity with one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof. In some embodiments, genetically engineered bacteria comprise a nucleic acid sequence encoding an adenosine degradation enzyme or adenosine transporter that has at least about 90% identity with one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof. In some embodiments, genetically engineered bacteria comprise a nucleic acid sequence encoding an adenosine degradation enzyme or adenosine transporter that has at least about 95% identity with one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof. In some embodiments, genetically engineered bacteria comprise a nucleic acid sequence encoding an adenosine degradation enzyme or adenosine transporter that is at least about 80%, at least about 85%, at least about 90%, at least about 95%, or at least about 99% homologous to one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77. In some embodiments, genetically engineered bacteria comprise a nucleic acid sequence encoding an adenosine degradation enzyme or adenosine transporter that comprises one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77. In some embodiments, genetically engineered bacteria comprise a nucleic acid sequence encoding an adenosine degradation enzyme or adenosine transporter that consists of one or more polynucleotide sequences selected from SEQ ID NO: 71, SEQ ID NO: 72, SEQ ID NO: 73, SEQ ID NO: 74, SEQ ID NO: 75, SEQ ID NO: 76, and/or SEQ ID NO: 77.

In some embodiments, genetically engineered bacteria comprise a nucleic acid sequence encoding an adenosine degradation enzyme or adenosine transporter that, but for the redundancy of the genetic code, encodes the same protein as a sequence selected from SEQ ID NO: 71, SEQ ID NO: 72, SEQ ID NO: 73, SEQ ID NO: 74, SEQ ID NO: 75, SEQ ID NO: 76, and/or SEQ ID NO: 77. In some embodiments, the genetically engineered bacteria comprise a nucleic acid encoding an adenosine degradation enzyme or adenosine transporter that, but for the redundancy of the genetic code, encodes a polypeptide that is at least about 80%, to the polypeptide encoded by a sequence selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof.

In some embodiments, the genetically engineered bacteria comprise a nucleic acid encoding an adenosine degradation enzyme or adenosine transporter that, but for the redundancy of the genetic code, encodes a polypeptide that is at least about 90% homologous to the polypeptide encoded by a sequence selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof.

In some embodiments, the genetically engineered bacteria comprise a nucleic acid encoding an adenosine degradation enzyme or adenosine transporter that, but for the redundancy of the genetic code, encodes a polypeptide that is at least about 95%, homologous to the polypeptide encoded by a sequence selected from SEQ ID NO: 71, SEQ ID NO: 72, SEQ ID NO: 73, SEQ ID NO: 74, SEQ ID NO: 75, SEQ ID NO: 76, and/or SEQ ID NO: 77, or a functional fragment thereof. In some embodiments, the genetically engineered bacteria comprise a nucleic acid encoding an adenosine degradation enzyme or adenosine transporter that, but for the redundancy of the genetic code, encodes a polypeptide that is at least about 80%, at least about 85%, at least about 90%, at least about 95%, or at least about 99% homologous to the polypeptide encoded by a sequence selected from SEQ ID NO: 71, SEQ ID NO: 72, SEQ ID NO: 73, SEQ ID NO: 74, SEQ ID NO: 75, SEQ ID NO: 76, and/or SEQ ID NO: 77.

In one specific embodiment, the genetically engineered bacteria comprise PfnrS-nupC integrated into the chromosome at HA1/2 (agaI/rsmI) region, PfnrS-xdhABC, integrated into the chromosome at HA9/10 (exo/cea) region, and PfnrS-add-xapA-deoD integrated into the chromosome at malE/K region.

In some embodiments, constructs comprise PfnrS (SEQ ID NO: 856), PfnrS-nupC (SEQ ID NO: 857), PfnrS-xdhABC (SEQ ID NO: 858), xdhABC (SEQ ID NO: 859), PfnrS-add-xapA-deoD (SEQ ID NO: 860), and add-xapA-deoD (SEQ ID NO: 861).

In some embodiments, genetically engineered bacteria comprise a nucleic acid sequence encoding an adenosine consuming construct that is at least about 80%, at least about 85%, at least about 90%, at least about 95%, or at least about 99% homologous to the a polynucleotide sequence selected from SEQ ID NO: 856, SEQ ID NO: 857, SEQ ID NO: 858, SEQ ID NO: 859, SEQ ID NO: 860, and/or SEQ ID NO: 861, or a variant or functional fragment thereof. In some embodiments, genetically engineered bacteria comprise a nucleic acid sequence encoding an adenosine consuming construct comprising one or more polynucleotide sequence(s) selected from SEQ ID NO: 856, SEQ ID NO: 857, SEQ ID NO: 858, SEQ ID NO: 859, SEQ ID NO: 860, and/or SEQ ID NO: 861. In some embodiments, genetically engineered bacteria comprise a nucleic acid sequence encoding an adenosine consuming construct consisting of one or more a polynucleotide sequence(s) selected from SEQ ID NO: 856, SEQ ID NO: 857, SEQ ID NO: 858, SEQ ID NO: 859, SEQ ID NO: 860, and/or SEQ ID NO: 861.

In some embodiments, genetically engineered bacteria comprise a nucleic acid sequence encoding an NupC. In one embodiment, the nucleic acid sequence encodes a NupC polypeptide, which has at least about 80% identity with SEQ ID NO: 78. In one embodiment, the nucleic acid sequence encodes a NupC polypeptide, which has at least about 90% identity with SEQ ID NO: 78. In another embodiment, the nucleic acid sequence encodes a NupC polypeptide, which has at least about 95% identity with SEQ ID NO: 78. Accordingly, in one embodiment, the nucleic acid sequence encodes a NupC polypeptide, which has at least about 85%, 86%, 87%, 88%, 89%, 90%, 91%, 92%, 93%, 94%, 95%, 96%, 97%, 98%, or 99% identity with SEQ ID NO: 78. In another embodiment, the nucleic acid sequence encodes a NupC polypeptide, which comprises a sequence which encodes SEQ ID NO: 78. In yet another embodiment, the nucleic acid sequence encodes a NupC polypeptide, which consists of SEQ ID NO: 78.

In some embodiments, genetically engineered bacteria comprise a nucleic acid sequence encoding XdhA. In one embodiment, the nucleic acid sequence encodes a XdhA polypeptide, which has at least about 80% identity with SEQ ID NO: 79. In one embodiment, the nucleic acid sequence encodes a XdhA polypeptide, which has at least about 90% identity with SEQ ID NO: 79. In another embodiment, the nucleic acid sequence encodes a XdhA polypeptide, which has at least about 95% identity with SEQ ID NO: 79. Accordingly, in one embodiment, the nucleic acid sequence encodes a XdhA polypeptide, which has at least about 85%, 86%, 87%, 88%, 89%, 90%, 91%, 92%, 93%, 94%, 95%, 96%, 97%, 98%, or 99% identity with SEQ ID NO: 79. In another embodiment, the nucleic acid sequence encodes a XdhA polypeptide, which comprises a sequence which encodes SEQ ID NO: 79. In yet another embodiment, the nucleic acid sequence encodes a XdhA polypeptide, which consists of a sequence which encodes SEQ ID NO: 79.

In some embodiments, genetically engineered bacteria comprise a nucleic acid sequence encoding XdhB. In one embodiment, the nucleic acid sequence encodes a XdhB polypeptide, which has at least about 80% identity with SEQ ID NO: 80. In one embodiment, the nucleic acid sequence encodes a XdhB polypeptide, which has at least about 90% identity with SEQ ID NO: 80. In another embodiment, the nucleic acid sequence encodes a XdhB polypeptide, which has at least about 95% identity with SEQ ID NO: 80. Accordingly, in one embodiment, the nucleic acid sequence encodes a XdhB polypeptide, which has at least about 85%, 86%, 87%, 88%, 89%, 90%, 91%, 92%, 93%, 94%, 95%, 96%, 97%, 98%, or 99% identity with SEQ ID NO: 80. In another embodiment, the nucleic acid sequence encodes a XdhB polypeptide, which comprises a sequence which encodes SEQ ID NO: 80. In yet another embodiment, the nucleic acid sequence encodes a XdhB polypeptide, which consists of a sequence which encodes SEQ ID NO: 80.

In some embodiments, genetically engineered bacteria comprise a nucleic acid sequence encoding XdhC. In one embodiment, the nucleic acid sequence encodes a XdhC polypeptide, which has at least about 80% identity with SEQ ID NO: 81. In one embodiment, the nucleic acid sequence encodes a XdhC polypeptide, which has at least about 90% identity with SEQ ID NO: 81. In another embodiment, the nucleic acid sequence encodes a XdhC polypeptide, which has at least about 95% identity with SEQ ID NO: 81. Accordingly, in one embodiment, the nucleic acid sequence encodes a XdhC polypeptide, which has at least about 85%, 86%, 87%, 88%, 89%, 90%, 91%, 92%, 93%, 94%, 95%, 96%, 97%, 98%, or 99% identity with SEQ ID NO: 81. In another embodiment, the nucleic acid sequence encodes a XdhC polypeptide, which comprises a sequence which encodes SEQ ID NO: 81. In yet another embodiment, the nucleic acid sequence encodes a XdhC polypeptide, which consists of a sequence which encodes SEQ ID NO: 81.

In some embodiments, genetically engineered bacteria comprise a nucleic acid sequence encoding Add. In one embodiment, the nucleic acid sequence encodes a Add polypeptide, which has at least about 80% identity with SEQ ID NO: 82. In one embodiment, the nucleic acid sequence encodes a Add polypeptide, which has at least about 90% identity with SEQ ID NO: 82. In another embodiment, the nucleic acid sequence encodes a Add polypeptide, which has at least about 95% identity with SEQ ID NO: 82. Accordingly, in one embodiment, the nucleic acid sequence encodes a Add polypeptide, which has at least about 85%, 86%, 87%, 88%, 89%, 90%, 91%, 92%, 93%, 94%, 95%, 96%, 97%, 98%, or 99% identity with SEQ ID NO: 82. In another embodiment, the nucleic acid sequence encodes a Add polypeptide, which comprises a sequence which encodes SEQ ID NO: 82. In yet another embodiment, the nucleic acid sequence encodes a Add polypeptide, which consists of a sequence which encodes SEQ ID NO: 82.

In some embodiments, genetically engineered bacteria comprise a nucleic acid sequence encoding XapA. In one embodiment, the nucleic acid sequence encodes a XapA polypeptide, which has at least about 80% identity with SEQ ID NO: 83. In one embodiment, the nucleic acid sequence encodes a XapA polypeptide, which has at least about 90% identity with SEQ ID NO: 83. In another embodiment, the nucleic acid sequence encodes a XapA polypeptide, which has at least about 95% identity with SEQ ID NO: 83. Accordingly, in one embodiment, the nucleic acid sequence encodes a XapA polypeptide, which has at least about 85%, 86%, 87%, 88%, 89%, 90%, 91%, 92%, 93%, 94%, 95%, 96%, 97%, 98%, or 99% identity with SEQ ID NO: 83. In another embodiment, the nucleic acid sequence encodes a XapA polypeptide, which comprises a sequence which encodes SEQ ID NO: 83. In yet another embodiment, the nucleic acid sequence encodes a XapA polypeptide, which consists of a sequence which encodes SEQ ID NO: 83.

In some embodiments, genetically engineered bacteria comprise a nucleic acid sequence encoding DeoD. In one embodiment, the nucleic acid sequence encodes a DeoD polypeptide, which has at least about 80% identity with SEQ ID NO: 84. In one embodiment, the nucleic acid sequence encodes a DeoD polypeptide, which has at least about 90% identity with SEQ ID NO: 84. In another embodiment, the nucleic acid sequence encodes a DeoD polypeptide, which has at least about 95% identity with SEQ ID NO: 84. Accordingly, in one embodiment, the nucleic acid sequence encodes a DeoD polypeptide, which has at least about 85%, 86%, 87%, 88%, 89%, 90%, 91%, 92%, 93%, 94%, 95%, 96%, 97%, 98%, or 99% identity with SEQ ID NO: 84. In another embodiment, the nucleic acid sequence encodes a DeoD polypeptide, which comprises a sequence which encodes SEQ ID NO: 84. In yet another embodiment, the nucleic acid sequence encodes a DeoD polypeptide, which consists of a sequence which encodes SEQ ID NO: 84.

In any of these embodiments, the bacteria genetically engineered to consume adenosine consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more adenosine than unmodified bacteria of the same bacterial subtype under the same conditions.

In any of these embodiments, the bacteria genetically engineered to consume adenosine produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more urate than unmodified bacteria of the same bacterial subtype under the same conditions.

In any of these embodiments, the genetically engineered bacteria increase the adenosine degradation rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the adenosine degradation rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase the degradation rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In some embodiments, the genetically engineered bacteria have an adenosine degradation rate of about 1.8-10 umol/hr/10\hat{ }9 cells when induced under low oxygen conditions. In one specific embodiment, the genetically engineered bacteria have an adenosine degradation rate of about 5-9 umol/hr/10\hat{ }9 cells. In one specific embodiment, the genetically engineered bacteria have an adenosine degradation rate of about 6-8 umol/hr/10\hat{ }9 cells.

In one embodiment, the genetically engineered bacteria increase the adenosine degradation by about 50% to 70% relative to unmodified bacteria of the same bacterial subtype under the same conditions, i.e., when induced under low oxygen conditions, after 1 hour. In one embodiment, the genetically engineered bacteria increase the adenosine degradation by about 55% to 65% relative to unmodified bacteria of the same bacterial subtype under the same conditions, i.e., when induced under low oxygen conditions after 1 hour. In one specific embodiment, the genetically engineered bacteria increase the adenosine degradation by about 55% to 60% relative to unmodified bacteria of the same bacterial subtype under the same conditions, i.e., when induced under low oxygen conditions, after 1 hour. In yet another embodiment, the genetically engineered bacteria increase the adenosine degradation by about 1.5-3 fold when induced under low oxygen conditions, after 1 hour. In one specific embodiment, the genetically engineered bacteria increase the adenosine degradation by about 2-2.5 fold when induced under low oxygen conditions, after 1 hour.

In one embodiment, the genetically engineered bacteria increase the adenosine degradation by about 85% to 100% relative to unmodified bacteria of the same bacterial subtype under the same conditions, i.e., when induced under low oxygen conditions, after 2 hours. In one embodiment, the genetically engineered bacteria increase the adenosine degradation by about 95% to 100% relative to unmodified bacteria of the same bacterial subtype under the same conditions, i.e., when induced under low oxygen conditions after 2 hours. In one specific embodiment, the genetically engineered bacteria increase the adenosine degradation by about 97% to 99% relative to unmodified bacteria of the same bacterial subtype under the same conditions, i.e., when induced under low oxygen conditions, after 2 hours.

In yet another embodiment, the genetically engineered bacteria increase the adenosine degradation by about 40-50 fold when induced under low oxygen conditions, after 2 hours. In one specific embodiment, the genetically engineered bacteria increase the adenosine degradation by about 44-48 fold when induced under low oxygen conditions, after 2 hours.

In one embodiment, the genetically engineered bacteria increase the adenosine degradation by about 95% to 100% relative to unmodified bacteria of the same bacterial subtype under the same conditions, i.e., when induced under low oxygen conditions, after 3 hours. In one embodiment, the genetically engineered bacteria increase the adenosine degradation by about 98% to 100% relative to unmodified bacteria of the same bacterial subtype under the same conditions, i.e., when induced under low oxygen conditions after 3 hours. In one specific embodiment, the genetically engineered bacteria increase the adenosine degradation by about 99% to 99% relative to unmodified bacteria of the same bacterial subtype under the same conditions, i.e., when induced under low oxygen conditions, after 3 hours. In yet another embodiment, the genetically engineered bacteria increase the adenosine degradation by about 100-1000 fold when induced under low oxygen conditions, after 3 hours. In yet another embodiment, the genetically engineered bacteria increase the adenosine degradation by about 1000-10000 fold when induced under low oxygen conditions, after 3 hours.

In one embodiment, the genetically engineered bacteria increase the adenosine degradation by about 95% to 100% relative to unmodified bacteria of the same bacterial subtype under the same conditions, i.e., when induced under low oxygen conditions, after 4 hours. In one embodiment, the genetically engineered bacteria increase the adenosine degradation by about 98% to 100% relative to unmodified bacteria of the same bacterial subtype under the same conditions, i.e., when induced under low oxygen conditions after 4 hours. In one embodiment, the genetically engineered bacteria increase the adenosine degradation by about 99% to 99% relative to unmodified bacteria of the same bacterial subtype under the same conditions, i.e., when induced under low oxygen conditions, after 4 hours. In yet another embodiment, the genetically engineered bacteria increase the adenosine degradation by about 100-1000 fold when induced under low oxygen conditions, after 4 hours. In yet another embodiment, the genetically engineered bacteria increase the adenosine degradation by about 1000-10000 fold when induced under low oxygen conditions, after 4 hours.

In any of these embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered microorganisms are capable of expressing any one or more of the described circuits for the degradation of adenosine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the degradation of adenosine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described adenosine degradation circuits are present on one or more plasmids (e.g., high copy or low copy) or are integrated into one or more sites in the microorganismal chromosome.

In any of these embodiments, the genetically engineered bacteria comprising gene sequence(s) encoding adenosine catabolic pathways and adenosine transporters described herein, further comprise gene sequence(s) encoding one or more further effector molecule(s), i.e., therapeutic molecule(s) or a metabolic converter(s). In any of these embodiments, the circuit encoding adenosine catabolic pathways and adenosine transporters, may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding adenosine catabolic pathways and adenosine transporters, may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding adenosine catabolic pathways and adenosine transporters, encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding adenosine catabolic pathways and adenosine transporters, encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

In some embodiments, the genetically engineered bacteria comprise a means for increasing the level of ATP in the tumor microenvironment, e.g., by increasing the production and secretion of ATP from the microorganism. In some embodiments, the genetically engineered bacteria comprise one or more means for reducing the levels of adenosine in the tumor microenvironment (e.g., by increasing the uptake of adenosine, by metabolizing and/or degrading adenosine), increasing the levels of ATP in the tumor microenvironment, and/or preventing or blocking the conversion of ATP to adenosine in the tumor microenvironment. In any of these embodiments, the genetically engineered bacterium is bacterium for intratumoral administration. In some embodiments, the genetically engineered bacterium comprises one or more genes for metabolizing adenosine, under the control of a promoter that is activated by low-oxygen conditions, by hypoxic conditions, or by inflammatory conditions, such as any of the promoters activated by said conditions and described herein. In some embodiments, the genetically engineered bacteria expresses one or more genes for metabolizing adenosine under the control of a cancer-specific promoter, a tissue-specific promoter, or a constitutive promoter, such as any of the promoters described herein.

### Arginine/Arginase I Metabolism

L-Arginine (L-Arg) is a nonessential amino acid that plays a central role in several biological systems including the immune response. L-Arginine is metabolized by arginase I, arginase II, and the inducible nitric oxide synthase. Arginase 1 hydrolyzes L-Arginine into urea and L-ornithine, the latter being the main substrate for the production of polyamines that are required for rapid cell cycle progression in malignancies. A distinct subpopulation of tumor-infiltrating myeloid-derived suppressor cells (MDSC), and not tumor cells themselves, have been shown to produce high levels of arginase I and cationic amino acid transporter 2B, which allow them to rapidly incorporate L-Arginine (L-Arg) and deplete extracellular L-Arg the tumor microenvironment. These cells are potent inhibitors of T-cell receptor expression and antigen-specific T-cell responses and potent inducers of regulatory T cells. Moreover, recent studies by Lanzavecchia and co-workers have shown that activated T cells also heavily consume L-arginine and rapidly convert it into downstream metabolites, which lead to a marked decrease in intracellular arginine levels after activation. In these studies, addition of exogenous L-arginine to T cell culture medium increased intracellular levels of free L-arginine in T cells, and moreover increased L-arginine levels caused pleiotropic effects on T cell activation, differentiation, and function, ranging from increased bioenergetics and survival to in vivo anti-tumor activity (Geiger et al., (2016) L-Arginine Modulates T Cell Metabolism and Enhances Survival and Anti-tumor Activity; Cell 167, 829-842, the contents of which is herein incorporated by reference in its entirety). Accordingly, bacteria engineered to produce and secrete arginine may be capable of promoting arginine uptake by T cells, leading to enhanced and more sustained T cell activation. Accordingly, in some embodiments, the genetically engineered bacteria of the disclosure are capable of producing arginine.

Recent findings suggest that the tumor microenvironment has a unique type of ammonia metabolism that is different from any other organ in the human body (Spinelli et al., Metabolic recycling of ammonia via glutamate dehydrogenase supports breast cancer biomass; Science 10.1126/science.aam9305 (2017)) Ammonia, which accumulates in the tumor microenvironment because tumors are poorly vascularized, is not a waste product but instead uniquely allows the tumor to reassimilate this ammonia as an important nitrogen source into metabolic pathways to support the high demand for amino acid synthesis in rapidly proliferating cancer cells. Additionally, Eng et al. (Eng et al., Ammonia Derived from Glutaminolysis Is a Diffusible Regulator of Autophagy; Science Signaling (2010); 3(118)ra31) found that ammonia liberated during glutaminolysis stimulates autophagy, which promotes cell fitness by recycling macromolecules into metabolic precursors needed for survival in rapidly proliferating cells. The authors propose that the liberation of ammonia from tumor cells engaged in glutaminolysis provides signal that promotes autophagy and, in turn, protects cells in different regions of the tumor from internally generated or environmental stress.

Accordingly, consumption of ammonia by the genetically engineered bacteria may reduce availability of ammonia for cancer metabolism or the promotion of autophagy in cancer cells. The disclosure described herein further provides genetically engineered bacteria that are capable of reducing excess ammonia and converting ammonia and/or nitrogen into alternate byproducts. In certain embodiments, the genetically engineered bacteria reduce excess ammonia and convert ammonia and/or nitrogen into alternate byproducts in the tumor microenvironment. In certain embodiments, the genetically engineered bacteria reduce excess ammonia by incorporating excess nitrogen in the tumor into molecules which reduce nitrogen availability to the tumor, e.g., arginine, citrulline, methionine, histidine, lysine, asparagine, glutamine, or tryptophan. In some embodiments, the genetically engineered bacteria reduce excess ammonia by incorporating excess nitrogen in the tumor into molecules which inhibit tumor growth or promote T cell activation, including, but not limited to, arginine. In some embodiments, the genetically engineered bacteria are capable of consuming ammonia and producing arginine.

In the arginine production circuit described herein below and in more detail in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, ammonia is taken up by a bacterium (e.g., E. coli Nissle), converted to glutamate, and glutamate is subsequently metabolized to arginine. Arginine then ultimately exits the bacterial cell. As such this circuit is suitable for the consumption of ammonia, reducing ammonia availability to the cancer cells in the tumor, and at the same time producing arginine, which promotes T cell activation and prevents immune suppression.

In some embodiments, the genetically engineered bacteria that produce L-Arginine and/or consume ammonia comprise one or more gene sequences encoding one or more enzymes of the L-Arginine biosynthetic pathway. In some embodiments, the genetically engineered bacteria comprise one or more gene sequences encoding one or more enzymes that are capable of incorporating ammonia into glutamate, and converting glutamate to arginine. In some embodiments, the genetically engineered bacteria comprise an Arginine operon. In some embodiments, the genetically engineered bacteria comprise the Arginine operon of E. coli. In some embodiments, the genetically engineered bacteria comprise the Arginine operon of another bacteria. In any of these embodiments, the arginine repressor (ArgR) optionally may be deleted, mutated, or modified so as to diminish or obliterate its repressor function.

“Arginine operon,” “arginine biosynthesis operon,” and “arg operon” are used interchangeably to refer to a cluster of one or more of the genes encoding arginine biosynthesis enzymes under the control of a shared regulatory region comprising at least one promoter and at least one ARG box. In some embodiments, the one or more genes are co-transcribed and/or co-translated.

“Mutant arginine regulon” or “mutated arginine regulon” is used to refer to an arginine regulon comprising one or more nucleic acid mutations that reduce or eliminate arginine-mediated repression of each of the operons that encode the enzymes responsible for converting glutamate to arginine in the arginine biosynthesis pathway, such that the mutant arginine regulon produces more arginine and/or intermediate byproduct than an unmodified regulon from the same bacterial subtype under the same conditions.

In bacteria such as Escherichia coli (E. coli), the arginine biosynthesis pathway is capable of converting glutamate to arginine in an eight-step enzymatic process described in in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, the contents of each of which is herein incorporated by reference in its entirety. All of the genes encoding these enzymes are subject to repression by arginine via its interaction with ArgR to form a complex that binds to the regulatory region of each gene and inhibits transcription. N-acetylglutamate synthetase is also subject to allosteric feedback inhibition at the protein level by arginine alone.

In some engineered bacteria, the arginine regulon includes, but is not limited to, argA, encoding N-acetylglutamate synthetase; argB, encoding N-acetylglutamate kinase; argC, encoding N-acetylglutamylphosphate reductase; argD, encoding acetylornithine aminotransferase; argE, encoding N-acetylornithinase; argG, encoding argininosuccinate synthase; argH, encoding argininosuccinate lyase; one or both of argF and argI, each of which independently encodes ornithine transcarbamylase; carA, encoding the small subunit of carbamoylphosphate synthase; carB, encoding the large subunit of carbamoylphosphate synthase; operons thereof; operators thereof; promoters thereof; ARG boxes thereof; and/or regulatory regions thereof. In some embodiments, the arginine regulon comprises argJ, encoding ornithine acetyltransferase (either in addition to or in lieu of N-acetylglutamate synthetase and/or N-acetylornithinase), operons thereof, operators thereof, promoters thereof, ARG boxes thereof, and/or regulatory regions thereof.

In some embodiments, the genetically engineered bacteria comprise an arginine biosynthesis pathway and are capable of producing arginine and/or consuming ammonia. In a more specific aspect, the genetically engineered bacteria comprise a mutant arginine regulon in which one or more operons encoding arginine biosynthesis enzyme(s) is derepressed to produce more arginine than unmodified bacteria of the same subtype under the same conditions. In some embodiments, the genetically engineered bacteria overproduce arginine. In some embodiments, the genetically engineered bacteria consume ammonia. In some embodiments, the genetically engineered bacteria overproduce arginine and consume ammonia.

Each operon is regulated by a regulatory region comprising at least one promoter and at least one ARG box, which control repression and expression of the arginine biosynthesis genes in said operon. In some embodiments, the genetically engineered bacteria comprise an arginine regulon comprising one or more nucleic acid mutations that reduce or eliminate arginine-mediated repression of one or more of the operons that encode the enzymes responsible for converting glutamate to arginine in the arginine biosynthesis pathway. Reducing or eliminating arginine-mediated repression may be achieved by reducing or eliminating ArgR repressor binding (e.g., by mutating or deleting the arginine repressor or by mutating at least one ARG box for each of the operons that encode the arginine biosynthesis enzymes) and/or arginine binding to N-acetylglutamate synthetase (e.g., by mutating the N-acetylglutamate synthetase to produce an arginine feedback resistant N-acetylglutamate synthase mutant, e.g., argAfbr).

In some embodiments, the reduction or elimination of arginine-mediated repression may be achieved by reducing or eliminating ArgR repressor binding, e.g., by mutating at least one ARG box for one or more of the operons that encode the arginine biosynthesis enzymes or by mutating or deleting the arginine repressor and/or by reducing or eliminating arginine binding to N-acetylglutamate synthetase (e.g., by mutating the N-acetylglutamate synthetase to produce an arginine feedback resistant N-acetylglutamate synthase mutant, e.g., argA′).

“ArgR” or “arginine repressor” is used to refer to a protein that is capable of suppressing arginine biosynthesis by regulating the transcription of arginine biosynthesis genes in the arginine regulon. Bacteria that “lack any functional ArgR” and “ArgR deletion bacteria” are used to refer to bacteria in which each arginine repressor has significantly reduced or eliminated activity as compared to unmodified arginine repressor from bacteria of the same subtype under the same conditions. ARG box refers to an nucleic acid sequence which comprises a consensus sequence, and which is known to occur with high frequency in one or more of the regulatory regions of argR, argA, argB, argC, argD, argE, argF, argG, argH, argI, argJ, carA, and/or carB.

In some embodiments, the genetically engineered bacteria comprise a mutant arginine regulon comprising one or more nucleic acid mutations in at least one ARG box for one or more of the operons that encode the arginine biosynthesis enzymes N-acetylglutamate kinase, N-acetylglutamylphosphate reductase, acetylornithine aminotransferase, N-acetylornithinase, ornithine transcarbamylase, argininosuccinate synthase, argininosuccinate lyase, and carbamoylphosphate synthase, such that the arginine regulon is derepressed and biosynthesis of arginine and/or an intermediate byproduct, e.g., citrulline, is enhanced. Such genetically engineered bacteria, mutant Arg boxes and exemplary mutant arginine regulons are described in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, the contents of each of which is herein incorporated by reference it its entirety.

In some embodiments, the genetically engineered bacteria lack a functional ArgR repressor and therefore ArgR repressor-mediated transcriptional repression of each of the arginine biosynthesis operons is reduced or eliminated. Genetically engineered bacteria according to the present disclosure that lack a functional ArgR repressor are described in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, the contents of each of which is herein incorporated by reference it its entirety. In some embodiments, the engineered bacteria comprise a mutant arginine repressor comprising one or more nucleic acid mutations such that arginine repressor function is decreased or inactive. In some embodiments, the genetically engineered bacteria do not have an arginine repressor (e.g., the arginine repressor gene has been deleted), resulting in depression of the regulon and enhancement of arginine and/or intermediate byproduct biosynthesis and/or increased ammonia consumption. Bacteria in which arginine repressor activity is reduced or eliminated can be generated by modifying the bacterial argR gene or by modifying the transcription of the argR gene. In some embodiments, each copy of a functional argR gene normally present in a corresponding wild-type bacterium is independently deleted or rendered inactive by one or more nucleotide deletions, insertions, or substitutions or is deleted.

In some embodiments, the genetically engineered bacteria comprise an arginine feedback resistant N-acetylglutamate synthase mutant, e.g., argA^{fbr} (see, e.g., Eckhardt et al., 1975; Rajagopal et al., 1998). Genetically engineered bacteria according to the present disclosure comprising argAfbr are described in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, the contents of each of which is herein incorporated by reference it its entirety. In some embodiments, the genetically engineered bacteria comprise a mutant arginine regulon comprising an arginine feedback resistant ArgA, and when the arginine feedback resistant ArgA is expressed, are capable of producing more arginine and/or an intermediate byproduct than unmodified bacteria of the same subtype under the same conditions. The feedback resistant argA gene can be present on a plasmid or chromosome, e.g., in one or more copies at one or more integration sites. Multiple distinct feedback resistant N-acetylglutamate synthetase proteins are known in the art and may be combined in the genetically engineered bacteria. In some embodiments, the argA gene is expressed under the control of a constitutive promoter. In some embodiments, the argA^{fbr} gene is expressed under the control of a promoter that is induced by tumor microenvironment. In some embodiments, the argA^{fbr} gene is expressed under the control of a promoter that is induced under low oxygen conditions, e.g., an FNR promoter.

The nucleic acid sequence of an exemplary argAfbr sequence is shown in SEQ ID NO: 102. The polypeptide sequence of an exemplary argAfbr sequence is shown in SEQ ID NO: 103.

In some embodiments, the genetically engineered bacteria comprise the nucleic acid sequence of SEQ ID NO: 102 or a functional fragment thereof. In some embodiments, the genetically engineered bacteria comprise a nucleic acid sequence that, but for the redundancy of the genetic code, encodes the same polypeptide as SEQ ID NO: 102 or a functional fragment thereof. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 102 or a functional fragment thereof, or a nucleic acid sequence that, but for the redundancy of the genetic code, encodes the same polypeptide as SEQ ID NO: 102 or a functional fragment thereof.

In some embodiments, the genetically engineered bacteria encode a polypeptide sequence of SEQ ID NO: 103 or a functional fragment thereof. In some embodiments, the genetically engineered bacteria encode a polypeptide sequence encodes a polypeptide, which contains one or more conservative amino acid substitutions relative to SEQ ID NO: 103 or a functional fragment thereof. In some embodiments, genetically engineered bacteria encode a polypeptide sequence that is at least about 80%, at least about 85%, at least about 90%, at least about 95%, or at least about 99% homologous to the DNA sequence of SEQ ID NO: 103 or a functional fragment thereof.

In some embodiments, arginine feedback inhibition of N-acetylglutamate synthetase is reduced by at least about 50%, at least about 60%, at least about 70%, at least about 80%, at least about 90%, or at least about 95% in the genetically engineered bacteria when the arginine feedback resistant N-acetylglutamate synthetase is active, as compared to a wild-type N-acetylglutamate synthetase from bacteria of the same subtype under the same conditions.

In some embodiments, the genetically modified bacteria comprising a mutant or deleted arginine repressor additionally comprise an arginine feedback resistant N-acetylglutamate synthase mutant, e.g., argA^{fbr}. In some embodiments, the genetically engineered bacteria comprise a feedback resistant form of ArgA, lack any functional arginine repressor, and are capable of producing arginine. In some embodiments, the argR gene is deleted in the genetically engineered bacteria. In some embodiments, the argR gene is mutated to inactivate ArgR function. In some embodiments, the genetically engineered bacteria comprise argA^{fbr} and deleted ArgR. In some embodiments, the deleted ArgR and/or the deleted argG is deleted from the bacterial genome and the argA^{fbr} is present in a plasmid. In some embodiments, the deleted ArgR is deleted from the bacterial genome and the argA chromosomally integrated.

In any of these embodiments, the bacteria genetically engineered to produce arginine and/or consume ammonia produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more arginine than unmodified bacteria of the same bacterial subtype under the same conditions.

In any of these embodiments, the bacteria genetically engineered to produce arginine and or consume ammonia consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more glutamate than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more glutamate than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these embodiments, the bacteria genetically engineered to produce arginine and or consume ammonia consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ammonia than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more ammonia than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ammonia than unmodified bacteria of the same bacterial subtype under the same conditions.

In any of these embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

Arginine producing strains and ammonia consuming strains are described in PCT/US2016/034200, filed May 25, 2016 and Ser. No. 15/164,828 filed May 25, 2016, published as US20160333326, and PCT/US2015/064140, filed Dec. 4, 2015, and U.S. Pat. No. 9,487,764, filed Dec. 4, 2015, the contents of each of which is herein incorporated by reference it its entirety.

In some embodiments, the genetically engineered microorganisms for the production of arginine and or consuming ammonia are capable of expressing any one or more of the described circuits in low-oxygen conditions, and/or in the presence of cancer and/or the tumor microenvironment, or tissue specific molecules or metabolites, and/or in the presence of molecules or metabolites associated with inflammation or immune suppression.

In some embodiments, any one or more of the described circuits for the production of arginine and or consumption of ammonia are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

In any of these embodiments, the genetically engineered bacteria comprising gene sequence(s) encoding arginine production and/or ammonia consumption circuitry further comprise gene sequence(s) encoding one or more further effector molecule(s), i.e., therapeutic molecule(s) or a metabolic converter(s). In any of these embodiments, the circuit encoding arginine production and/or ammonia consumption circuitry may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding arginine production and/or ammonia consumption circuitry may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding arginine production and/or ammonia consumption circuitry encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding arginine production and/or ammonia consumption circuitry encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Th1/CD8-Attracting Chemokines

Chemokines are critical for attracting and recruiting immune cells, e.g., those that activate immune response and those that induce cancer cell apoptosis. Target cells of chemokines express corresponding receptors to which chemokines bind and mediate function. Therefore, the receptors of CC and CXC chemokine are referred to as CCRs and CXCRs, respectively. CC chemokines bind to CC chemokine receptors, and CXC chemokines bind to CXC chemokine receptors. Most receptors usually bind to more than one chemokine, and most chemokines usually bind to more than one receptor.

The chemokine interferon-γ inducible protein 10 kDa (CXCL10) is a member of the CXC chemokine family which binds to the CXCR3 receptor to exert its biological effects. CXCL10 is involved in chemotaxis, induction of apoptosis, regulation of cell growth and mediation of angiostatic effects. CXCL10 is associated with a variety of human diseases including infectious diseases, chronic inflammation, immune dysfunction, tumor development, metastasis and dissemination. More importantly, CXCL10 has been identified as a major biological marker mediating disease severity and may be utilized as a prognostic indicator for various diseases. In this review, we focus on current research elucidating the emerging role of CXCL10 in the pathogenesis of cancer. Understanding the role of CXCL10 in disease initiation and progression may provide the basis for developing CXCL10 as a potential biomarker and therapeutic target for related human malignancies.

CXCL10 and CXCL9 each specifically activate a receptor, CXCR3, which is a seven trans-membrane-spanning G protein-coupled receptor predominantly expressed on activated T lymphocytes (Th1), natural killer (NK) cells, inflammatory dendritic cells, macrophages and B cells. The interferon-induced angiostatic CXC chemokines and interferon-inducible T-cell chemoattractant (I-TAC/CXCL11), also activate CXCR3. These CXC chemokines are preferentially expressed on Th1 lymphocytes.

Immune-mediated, tissue-specific destruction has been associated with Th1 polarization, related chemokines (CXCR3 and CCR5 ligands, such as CXCL10 and CXCL9), and genes associated with the activation of cytotoxic mechanisms. Other studies have shown that long disease-free survival and overall survival in cancers such as early-stage breast cancer, colorectal, lung, hepatocellular, ovarian, and melanoma are consistently associated with the activation of T helper type 1 (Th1) cell-related factors, such as IFN-gamma, signal transducers and activator of transcription 1 (STA1), IL-12, IFN-regulatory factor 1, transcription factor T-bet, immune effector or cytotoxic factors (granzymes), perforin, and granulysin, CXCR3 and CCR6 ligand chemokines (CXCL9, CXCL10, and CCL5), other chemokines (CXCL1 and CCL2), and adhesion molecules (MADCAM1, ICAM1, VCAM1). Chemoattraction and adhesion has been shown to play a critical role in determining the density of intratumoral immune cells.

Other studies have shown that up-regulation of CXCL9, CXCL10, and CXCL11 is predictive of treatment responsiveness (particular responsive to adoptive-transfer therapy). Still other studies have shown that chemokines that drive tumor infiltration by lymphocytes predicts survival of patients with hepatocellular carcinoma.

It is now recognized that cancer progression is regulated by both cancer cell-intrinsic and microenvironmental factors. It has been demonstrated that the presence of T helper 1 (Th1) and/or cytotoxic T cells correlates with a reduced risk of relapse in several cancers and that a pro-inflammatory tumor microenvironment correlates with prolonged survival in a cohort of patients with hepatocellular carcinoma. CXCL10, CCL5, and CCL2 expression has been shown to correlate with tumor infiltration by Th1, CD8⁺ T cells, and natural killer cells. Data shows that CXCL10, CCL5, and CCL2 are the main chemokines attracting Th1, CD8⁺ T cells, and NK cells into the tumor microenvironment. Also, CXCL10 and TLR3 (induces CXCL 10, CCL5, and CCL2) expression correlates with cancer cell apoptosis.

C-X-C motif chemokine 10 (CXCL10), also known as Interferon gamma-induced protein 10 (IP-10) or small-inducible cytokine B10 is an 8.7 kDa protein that in humans is encoded by the CXCL10 gene. CXCL10 is a small cytokine belonging to the CXC chemokine family which is secreted by several cell types in response to IFN-γ, including monocytes, endothelial cells and fibroblasts. CXCL10 plays several roles, including chemoattraction for monocytes/macrophages, T cells, NK cells, and dendritic cells, promotion of T cell adhesion to endothelial cells, antitumor activity, and inhibition of bone marrow colony formation and angiogenesis. This chemokine elicits its effects by binding to the cell surface chemokine receptor CXCR3.

Under proinflammatory conditions CXCL10 is secreted from a variety of cells, such as leukocytes, activated neutrophils, eosinophils, monocytes, epithelial cells, endothelial cells, stromal cells (fibroblasts) and keratinocytes in response to IFN-γ. This crucial regulator of the interferon response, preferentially attracts activated Th1 lymphocytes to the area of inflammation and its expression is associated with Th1 immune responses. CXCL10 is also a chemoattractant for monocytes, T cells and NK cells. (Chew et al., Gut, 2012, 61:427-438. Still other studies have shown that immune-protective signature genes, such as Th1-type chemokines CXCL10 and CXCL9, may be epigenetically silenced in cancer. (Peng et al., Nature, 2015, doi:10.1038/nature 15520).

Chemokine (C-X-C motif) ligand 9 (CXCL9) is a small cytokine belonging to the CXC chemokine family that is also known as Monokine induced by gamma interferon (MIG). CXCL9 is a T-cell chemoattractant (Th1/CD8-attracting chemokine) which is induced by IFN-γ. It is closely related to two other CXC chemokines, CXCL10 and CXCL11. CXCL9, CXCL10 and CXCL11 all elicit their chemotactic functions by interacting with the chemokine receptor CXCR3.

In some embodiments, the engineered bacteria comprise gene sequence encoding one or more chemokines that are Th1/CD8-attracting chemokines. In some embodiments, the engineered bacteria comprise gene sequence encoding one or more chemokines that are CXCR3 ligand chemokines. In some embodiments, the engineered bacteria comprise gene sequence encoding one or more chemokines that are CCR5 ligand chemokines. In some embodiments, the engineered bacteria comprise gene sequence encoding one or more copies of CXCL10.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CXCL10 than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more CXCL10 than unmodified bacteria of the same bacterial subtype under the same conditions. In yet another embodiment, the genetically engineered bacteria produce at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CXCL10 than unmodified bacteria of the same bacterial subtype under the same conditions.

In any of these embodiments, the bacteria genetically engineered to produce CXCL10 secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CXCL10 than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more CXCL10 than unmodified bacteria of the same bacterial subtype under the same conditions. In yet another embodiment, the genetically engineered bacteria secrete at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CXCL10 than unmodified bacteria of the same bacterial subtype under the same conditions.

In some embodiments, the bacteria genetically engineered to secrete CXCL10 are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete CXCL10 are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete CXCL10 are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL10 are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL10 are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL10 are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to produce CXCL10 are capable of attracting activated Th1 lymphocytes to at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In some embodiments, the bacteria genetically engineered to CXCL10 are capable of attracting activated Th1 lymphocytes to at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In yet another embodiment, the genetically engineered bacteria attract activated Th1 lymphocytes to at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria attract activated Th1 lymphocytes to about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL10 are capable of promoting chemotaxis of T cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In yet another embodiment, the genetically engineered bacteria promote chemotaxis of T cells by at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria promote chemotaxis of T cells about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL10 are capable of promoting chemotaxis of NK cells to at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In yet another embodiment, the genetically engineered bacteria promote chemotaxis of NK cells by at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria promote chemotaxis of NK cells at least about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL10 are capable of binding to CXCR3 by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater affinity as compared to an unmodified bacteria of the same subtype under the same conditions. In yet another embodiment, the genetically engineered bacteria bind to CXCR3 with at least about 1.0-1.2-fold, 1.2-1.4-fold, 1.4-1.6-fold, 1.6-1.8-fold, 1.8-2-fold, or two-fold greater affinity than unmodified bacteria of the same bacterial subtype under the same conditions. In yet another embodiment, the genetically engineered bacteria are capable of promoting chemotaxis of T cells to at least about a three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the genetically engineered bacteria comprise a gene sequence encoding a CXCL10 polypeptide, or a fragment or functional variant thereof. In one embodiment, the gene sequence encoding CXCL10 polypeptide has at least about 80% identity with a sequence selected from SEQ ID NO: 1207 or SEQ ID NO: 1208. In another embodiment, the gene sequence encoding CXCL10 polypeptide has at least about 85% identity with a sequence selected from SEQ ID NO: 1207 or SEQ ID NO: 1208. In one embodiment, the gene sequence encoding CXCL10 polypeptide has at least about 90% identity with a sequence selected from SEQ ID NO: 1207 or SEQ ID NO: 1208. In one embodiment, the gene sequence CXCL10 polypeptide has at least about 95% identity with a sequence selected from SEQ ID NO: 1207 or SEQ ID NO: 1208. In another embodiment, the gene sequence encoding CXCL10 polypeptide has at least about 96%, 97%, 98%, or 99% identity with a sequence selected from SEQ ID NO: 1207 or SEQ ID NO: 1208. Accordingly, in one embodiment, the gene sequence encoding CXCL10 polypeptide has at least about 80%, 81%, 82%, 83%, 84%, 85%, 86%, 87%, 88%, 89%, 90%, 91%, 92%, 93%, 94%, 95%, 96%, 97%, 98%, or 99% identity with a sequence selected from SEQ ID NO: 1207 or SEQ ID NO: 1208. In another embodiment, the gene sequence encoding CXCL10 polypeptide comprises a sequence selected from SEQ ID NO: 1207 or SEQ ID NO: 1208. In yet another embodiment, the gene sequence encoding CXCL10 polypeptide consists of a sequence selected from SEQ ID NO: 1207 or SEQ ID NO: 1208. In any of these embodiments wherein the genetically engineered bacteria encode CXCL10, one or more of the sequences encoding a secretion tag may be removed and replaced by a different tag.

In some embodiments, the genetically engineered bacteria comprise a gene sequence encoding a CXCL10 polypeptide having at least about 80% identity with a sequence selected from SEQ ID NO: 1205 or SEQ ID NO: 1206. In some embodiments, the genetically engineered bacteria comprise a gene sequence encoding a CXCL10 polypeptide that has about having at least about 90% identity with a sequence selected from SEQ ID NO: 1205 or SEQ ID NO: 1206. In some embodiments, the genetically engineered bacteria comprise a gene sequence encoding CXCL10 polypeptide that has about having at least about 95% identity with a sequence selected from SEQ ID NO: 1205 or SEQ ID NO: 1206. In some embodiments, the genetically engineered bacteria comprise a gene sequence encoding a CXCL10 polypeptide that has about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NO: 1205 or SEQ ID NO: 1206, or a functional fragment thereof. In another embodiment, the CXCL10 polypeptide comprises a sequence selected from SEQ ID NO: 1205 or SEQ ID NO: 1206. In yet another embodiment, the CXCL10 polypeptide expressed by the genetically engineered bacteria consists of a sequence selected from SEQ ID NO: 1205 or SEQ ID NO: 1206. In any of these embodiments wherein the genetically engineered bacteria encode CXCL10 polypeptide, the secretion tag may be removed and replaced by a different secretion tag.

In some embodiments, the genetically engineered microorganisms are capable of expressing any one or more of the described CXCL10 circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding CXCL10 are controlled by a promoter inducible by such conditions and/or inducers. In some embodiments, the gene sequences(s) encoding CXCL10 are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, the CXCL10 is secreted. In some embodiments, the genetically engineered bacteria comprising the gene sequence(s) encoding CXCL10 comprise a secretion tag selected from PhoA, OmpF, cvaC, TorA, FdnG, DmsA, and PelB. In some embodiments, the secretion tag is PhoA. In some embodiments, the genetically engineered bacteria further comprise one or more deletions in an outer membrane protein selected from 1pp, n1P, tolA, and PAL. In some embodiments, the deleted or mutated outer membrane protein is PAL. In some embodiments, the genetically engineered bacteria comprising gene sequence(s) for the production of CXCL10 further comprise gene sequence(s) encoding IL-15. In some embodiments, IL-15 is secreted. In some embodiments, the gene sequence(s) encoding IL-15 comprise a secretion tag selected from PhoA, OmpF, cvaC, TorA, FdnG, DmsA, and PelB. In some embodiments, the secretion tag is PhoA. In some embodiments, the genetically engineered bacteria further comprise one or more deletions in an outer membrane protein selected from 1pp, n1P, tolA, and PAL. In some embodiments, the deleted or mutated outer membrane protein is PAL.

In some embodiments, any one or more of the described genes sequences encoding CXCL10 are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

In any of these embodiments, the genetically engineered bacteria comprising gene sequence(s) encoding CXCL10 further comprise gene sequence(s) encoding one or more further effector molecule(s), i.e., therapeutic molecule(s) or a metabolic converter(s). In any of these embodiments, the circuit encoding CXCL10 may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding CXCL10 may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding CXCL10 encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding CXCL10 encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CXCL9 than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more CXCL9 than unmodified bacteria of the same bacterial subtype under the same conditions. In yet another embodiment, the genetically engineered bacteria produce at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CXCL9 than unmodified bacteria of the same bacterial subtype under the same conditions.

In any of these embodiments, the bacteria genetically engineered to produce CXCL9 secrete at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more CXCL9 than unmodified bacteria of the same bacterial subtype under the same conditions. In yet another embodiment, the genetically engineered bacteria secrete at least about 1.0-1.2-fold, 1.2-1.4-fold, 1.4-1.6-fold, 1.6-1.8-fold, 1.8-2-fold, or two-fold more CXCL9 than unmodified bacteria of the same bacterial subtype under the same conditions. In yet another embodiment, the genetically engineered bacteria secrete at least about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more CXCL9 than unmodified bacteria of the same bacterial subtype under the same conditions.

In some embodiments, the bacteria genetically engineered to secrete at least about CXCL9 are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete CXCL9 are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to secrete CXCL9 are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL9 are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL9 are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce CXCL9 are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the bacteria genetically engineered to produce CXCL9 are capable of attracting activated Th1 lymphocytes to at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater extent as compared to an unmodified bacteria of the same subtype under the same conditions. In yet another embodiment, the genetically engineered bacteria attract activated Th1 lymphocytes to at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria attract activated Th1 lymphocytes to at least about a three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL9 are capable of promoting chemotaxis of T cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In yet another embodiment, the genetically engineered bacteria promote chemotaxis of T cells to at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria promote chemotaxis of T cells to a at least about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL9 are capable of promoting chemotaxis of NK cells by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In yet another embodiment, the genetically engineered bacteria promote chemotaxis of NK cells to at least about 1.0-1.2-fold, 1.2-1.4-fold, 1.4-1.6-fold, 1.6-1.8-fold, 1.8-2-fold, or two-fold greater extent than unmodified bacteria of the same bacterial subtype under the same conditions. In yet another embodiment, the genetically engineered bacteria promote chemotaxis of NK cells to a three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater extent than unmodified bacteria of the same bacterial subtype under the same conditions.

In some embodiments, the bacteria genetically engineered to produce CXCL9 are capable of binding to CXCR3 by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or greater affinity as compared to an unmodified bacteria of the same subtype under the same conditions. In yet another embodiment, the genetically engineered bacteria bind to CXCR3 with at least 1.0-1.2-fold, 1.2-1.4-fold, 1.4-1.6-fold, 1.6-1.8-fold, 1.8-2-fold, or two-fold greater affinity than unmodified bacteria of the same bacterial subtype under the same conditions. In yet another embodiment, the genetically engineered bacteria are capable of binding to CXCR3 with at least about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold greater affinity than unmodified bacteria of the same bacterial subtype under the same conditions.

In some embodiments, the genetically engineered microorganisms are capable of expressing any one or more of the described CXCL9 circuits in low-oxygen conditions, and/or in the presence of cancer and/or in the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding CXCL9 are controlled by a promoter inducible by such conditions and/or inducers. In some embodiments, the gene sequences(s) encoding CXCL9 are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, any one or more of the described genes sequences encoding CXCL9 are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

In any of these embodiments, the genetically engineered bacteria comprising gene sequence(s) encoding CXCL9 further comprise gene sequence(s) encoding one or more further effector molecule(s), i.e., therapeutic molecule(s) or a metabolic converter(s). In any of these embodiments, the circuit encoding CXCL9 may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding CXCL9 may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding CXCL9 encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding CXCL9 encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Stromal Modulation

The accumulation of extracellular matrix (ECM) components can distort the normal architecture of tumor and stromal tissue, causing an abnormal configuration of blood and lymphatic vessels. One factor that may contribute to the therapeutic resistance of a tumor is the rigidity of the ECM that significantly compresses blood vessels, resulting in reduced perfusion (due to constraints on diffusion and convection) that ultimately impedes the delivery of therapeutics to tumor cells. One strategy to reduce vessel compression in the stroma and assist in drug delivery is to enzymatically break down the ECM scaffold, which in some stromal tumor environments consist of fibroblasts, immune cells, and endothelial cells imbedded within a dense and complex ECM with abundant Hyaluronan or Hyaluronic acid (HA). HA is a large linear glycosaminoglycan (GAG) composed of repeating N-acetyl glucosamine and glucuronic acid units that retains water due to its high colloid osmotic pressure. HA is believed to play a role in tumor stroma formation and maintenance. Enzymatic HA degradation by hyaluronidase (PEGPH20; rHuPH20) has been shown to decrease interstitial fluid pressure in mouse pancreatic ductal adenocarcinoma (PDA) tumors with a concomitant observation in vessel patency, drug delivery, and survival (Provenzano et al. Cancer Cell, 2012, 21:418-429; Thompson et al., Mol Cancer Ther, 2010, 9:3052-64). It is believed that PEGPH20 liberates water bound to HA by cleaving the extended polymer into substituent units. The release of trapped water decreases the interstitial fluid pressure to a range of 20-30 mmHg, enabling collapsed arterioles and capillaries to open (Provenzano et al.).

In some embodiments, the engineered bacteria comprise gene sequence encoding one or more molecules that modulate the stroma. In some embodiments, the engineered bacteria comprise gene sequence encoding one or more copies of an enzyme that degrades Hyaluronan or Hyaluronic acid (HA). In some embodiments, the engineered bacteria comprise gene sequence encoding one or more copies of hyaluronidase.

In any of these embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more hyaluronidase than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more hyaluronidase than unmodified bacteria of the same bacterial subtype under the same conditions. In yet another embodiment, the genetically engineered bacteria produce three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more hyaluronidase than unmodified bacteria of the same bacterial subtype under the same conditions.

In any of these embodiments, the bacteria genetically engineered to produce hyaluronidase degrade 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more hyaluronan than unmodified bacteria of the same bacterial subtype under the same conditions.

In yet another embodiment, the genetically engineered bacteria degrade 1.0-1.2-fold, 1.2-1.4-fold, 1.4-1.6-fold, 1.6-1.8-fold, 1.8-2-fold, or two-fold more hyaluronan than unmodified bacteria of the same bacterial subtype under the same conditions. In yet another embodiment, the genetically engineered bacteria degrade three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more hyaluronan than unmodified bacteria of the same bacterial subtype under the same conditions. In one embodiment, the genetically engineered bacteria comprising one or more genes encoding hyaluronidase for secretion are capable of degrading hyaluronan to about the same extent as recombinant hyaluronidase at the same concentrations under the same conditions.

In some embodiments, the bacteria genetically engineered to secrete hyaluronidase are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete hyaluronidase are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to secrete hyaluronidase are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce hyaluronidase are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce hyaluronidase are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In some embodiments, the bacteria genetically engineered to produce hyaluronidase are capable of increasing the response rate by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria comprise hyaluronidase gene sequence(s) encoding one or more polypeptide(s) selected from SEQ ID NO: 1127, SEQ ID NO: 1128, SEQ ID NO:1129, SEQ ID NO: 1130, SEQ ID NO: 1131 or functional fragments thereof. In some embodiments, genetically engineered bacteria comprise a gene sequence encoding a polypeptide that is at least about 80%, at least about 85%, at least about 90%, at least about 95%, or at least about 99% identity to one or more polypeptide(s) selected from selected from SEQ ID NO: 1127, SEQ ID NO: 1128, SEQ ID NO:1129, SEQ ID NO: 1130, SEQ ID NO: 1131 or a functional fragment thereof. In some specific embodiments, the polypeptide comprises one or more polypeptide(s) selected form selected from SEQ ID NO: 1127, SEQ ID NO: 1128, SEQ ID NO:1129, SEQ ID NO: 1130, SEQ ID NO: 1131. In other specific embodiments, the polypeptide consists of one or more polypeptide(s) of selected from selected from SEQ ID NO: 1127, SEQ ID NO: 1128, SEQ ID NO:1129, SEQ ID NO: 1130, SEQ ID NO: 1131. In certain embodiments, the hyaluronidase sequence has at least about 80%, 85%, 86%, 87%, 88%, 89%, 90%, 91%, 92%, 93%, 94%, 95%, 96%, 97%, 98%, or 99% identity with one or more polynucleotides selected from SEQ ID NO: 1122, SEQ ID NO: 1123, SEQ ID NO: 1224, SEQ ID NO: 1225, SEQ ID NO: 1226 or a functional fragment thereof. In some specific embodiments, the gene sequence comprises one or more sequences selected from SEQ ID NO: 1127, SEQ ID NO: 1128, SEQ ID NO:1129, SEQ ID NO: 1130, SEQ ID NO: 1131. In other specific embodiments, the gene sequence consists of one or more polynucleotides selected from SEQ ID NO: 1127, SEQ ID NO: 1128, SEQ ID NO:1129, SEQ ID NO: 1130, SEQ ID NO: 1131.

In some embodiments, the engineered bacteria comprise gene sequence encoding one or more copies of human hyaluronidase. In some embodiments, the hyaluronidase is leech hyaluronidase. In any of these embodiments, the gene sequences comprising the hyaluronidase further encode a secretion tag selected from PhoA, OmpF, cvaC, TorA, FdnG, DmsA, and PelB. In some embodiments, the secretion tag is at the N terminus of the hyaluronidase polypeptide sequence and at the 5′ end of the hyaluronidase coding sequence. In some embodiments, the secretion tag is at the C terminus of the hyaluronidase polypeptide sequence and at the 3′ end of the hyaluronidase coding sequence. In one embodiment, the secretion tag is PhoA. In some embodiments, the genetically engineered bacteria encode hyaluronidase for secretion. In some embodiments, the genetically engineered bacteria encode hyaluronidase for display on the bacterial cell surface. In some embodiments, the genetically engineered bacteria further comprise one or more deletions in an outer membrane protein selected from 1pp, nlP, tolA, and PAL. In some embodiments, the deleted or mutated outer membrane protein is PAL.

In some embodiments, the genetically engineered microorganisms are capable of expressing any one or more of the described stromal modulation circuits or gene sequences, e.g., hyaluronidase circuits, in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding stromal modulation circuits, e.g., hyaluronidase circuits, are controlled by a promoter inducible by such conditions and/or inducers in vivo and/or in vitro. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In some embodiments, any one or more of the described stromal modulation gene sequences, e.g., hyaluronidase gene sequences, are present on one or more plasmids (e.g., high copy or low copy) or are integrated into one or more sites in the microorganismal chromosome.

In any of these embodiments, the genetically engineered bacteria comprising gene sequence(s) encoding stromal modulation effectors, e.g., hyaluronidase, further comprise gene sequence(s) encoding one or more further effector molecule(s), i.e., therapeutic molecule(s) or a metabolic converter(s). In any of these embodiments, the circuit encoding stromal modulation effectors, e.g., hyaluronidase, may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein.

In any of these embodiments, the gene sequence(s) encoding stromal modulation effectors, e.g., hyaluronidase, may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding stromal modulation effectors, e.g., hyaluronidase, encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the the gene sequence(s) encoding stromal modulation effectors, e.g., hyaluronidase, encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

Also, in some embodiments, the genetically engineered microorganisms are capable of expressing any one or more of the described stromal modulation, e.g., hyaluronidase circuits, and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the genetically engineered bacteria may be administered alone or in combination with one or more immune checkpoint inhibitors described herein, including but not limited anti-CTLA4, anti-PD1, or anti-PD-L1 antibodies.

### Other Immune Modulators

Other immune modulators include therapeutic nucleic acids (RNA and DNA), for example, RNAi molecules (such as siRNA, miRNA, dsRNA), mRNAs, antisense molecules, aptamers, and CRISPER/Cas 9 molecules as described in International Patent Application PCT/US2017/013072, filed Jan. 11, 2017, published as WO2017/123675, the contents of which is herein incorporated by reference in its entirety. Thus, in some embodiments, the genetically engineered bacteria comprise sequence(s) for producing one or immune modulators that are RNA or DNA immune modulators, e.g., including nucleic acid molecules selected from RNAi molecules (siRNA, miRNA, dsRNA), mRNAs, antisense molecules, aptamers, and CRISPR/Cas 9 molecules. Such molecules are exemplified and discussed in the references provided herein below.

In any of these embodiments, these circuits may be combined with a circuit for the production of one or more immune initiators (e.g., a STING agonist as described herein the same or a different bacterial strain (combination circuit or mixture of strains).

### Combinations of Immune Initiators and Immune Sustainers

In some embodiments, the circuitry expressed by the genetically engineered bacteria is selected to combine multiple mechanisms. For example, by activating multiple orthogonal immunomodulatory pathways in the tumor microenvironment, immunologically cold tumors are transformed into immunologically hot tumors. Multiple effectors can be selected which have an impact on different components of the immune response. Different immune response components which can be targeted by the effectors expressed by the genetically engineered bacteria include immune initiation and immune augmentation and T cell expansion (immune sustenance).

In some embodiments, a first modified microorganism producing at least a first immune modulator, e.g., an immune initiator or an immune sustainer, may be administered in combination with, e.g., before, at the same time as, or after, a second modified microorganism producing at least a second immune modulator, e.g., an immune initiator or an immune sustainer. In other embodiments, one or more immune modulators may be administered in combination with, e.g., before, at the same time as, or after, a modified microorganism capable of producing a second immune modulator(s). For example, one or more immune initiators may be administered in combination with, e.g., before, at the same time as, or after, a modified microorganism capable of producing one or more immune sustainers. In another embodiment, one or more immune sustainers may be administered in combination with, e.g., before, at the same time as, or after, a modified microorganism capable of producing one or more immune initiators. Alternatively, one or more first immune initiators may be administered in combination with, e.g., before, at the same time as, or after, a modified microorganism capable of producing one or more second immune initiators. Alternatively, one or more first immune sustainers may be administered in combination with, e.g., before, at the same time as, or after, a modified microorganism capable of producing one or more second immune sustainers. In some embodiments, an immune initiator and/or an immune sustainer may further be combined with a stromal modulator, e.g., hyaluronidase.

In some embodiments, one or more microorganisms are genetically engineered to express gene sequence(s) encoding one or more immunomodulatory effectors or combinations of two or more these effectors. In some embodiments, the genetically engineered bacteria comprise circuitry encoding one or more immunomodulatory effectors or combinations of two or more these effectors. Alternatively, the disclosure provides a composition comprising a combination (e.g., two or more) of different or separate genetically engineered bacteria, each bacteria encoding one or more one or more immunomodulatory effectors. Such distinct or different bacterial strains can be administered concurrently or sequentially.

In some embodiments, the genetically engineered bacteria comprise circuitry that can modulate immune initiation (including e.g., activation and priming) and immune sustenance (including e.g., immune augmentation or T cell expansion). Accordingly, in some embodiments, the genetically engineered bacteria comprise circuitry or gene sequences encoding one or more immune initiators and one or more immune sustainers.

Alternatively, the disclosure provides a composition comprising a combination (e.g., two or more) of different genetically engineered bacteria, each bacteria encoding one or more immune initiators and/or one or more immune sustainers. Such distinct or different bacterial strains can be administered concurrently or sequentially.

Each combination of gene sequence(s), circuits, effectors, immune modulators, immune initiators or immune sustainers described herein can either be provided as combination circuitry in one bacterial strain or alternatively in two or more different or separate bacterial strains each expressing one or more gene sequence(s), circuits, effectors, immune modulators, immune initiators or immune sustainers of the combination. For example, one or more genetically engineered bacteria comprising circuitry for the production of an immune initiator and gene circuitry for the production of an immune sustainer can be provided in one strain comprising both circuits or in two or more strains, each comprising at least one of the circuits.

In some embodiments of the disclosure, in which a microorganism genetically engineered to express an immune initiator circuit and immune sustainer circuit, the microorganism first produces higher levels of immune stimulator and at a later time point immune sustainer. In certain embodiments, the one or more gene sequences are under the control of inducible promoters known in the art or described herein. For example, such inducible promoters may be induced under low-oxygen conditions, such as an FNR promoter. In some embodiments, the one or more gene sequence(s) are operably linked to a directly or indirectly inducible promoter that is induced under inflammatory conditions (e.g., RNS, ROS), as described herein. In other embodiments, the promoters are induced in the presence of certain molecules or metabolites, e.g., in the presence of molecules or metabolites associated with the tumor microenvironment and/or with immune suppression. In some embodiments, the promoters are induced in certain tissue types.

In some embodiments, promoters are induced in the presence of certain gut-specific or tumor-specific molecules or metabolites. In some embodiments, the promoters are induced in the presence of some other metabolite that may or may not be present in the gut or the tumor, such as arabinose, cumate, and salicylate or another chemical or nutritional inducer known in the art or described herein. In certain embodiments, the one or more cassettes are under the control of constitutive promoters described herein or known in the art, e.g., whose expression can be fine-tuned using ribosome binding sites of different strengths. Such microorganisms optionally also comprise an auxotrophic modification, e.g., an auxotrophic modification amino acid or nucleotide metabolism. Non-limiting examples of genes which may be modified are ThyA and DapA or both (ΔDapA or ΔThyA or both).

In some embodiments, expression of the immune initiator is under control of a promoter induced by a chemical inducer. In some embodiments, immune sustainer is under control of a promoter induced by a chemical inducer. In some embodiments, both immune initiator and immune sustainer are under control of promoters which are induced by a chemical inducer. The inducer (inducing immune stimulator expression) and a second inducer (inducing immune sustainer expression) may be the same or different inducers. First inducer and second inducer may be administered sequentially or concurrently. In some embodiments, immune sustainers and/or immune initiators may be induced under in vivo conditions, e.g., by conditions of the gut or the tumor microenvironment (e.g., low oxygen, certain nutrients, etc.), conditions during cell culture or in vitro growth, or chemical inducers (e.g., arabinose, cumate, and salicylate, IPTG or other chemical inducers described herein), which can be employed in vitro or in vivo.

In some embodiments, the immune initiator is controlled by or directly or indirectly linked to an inducible promoter and immune sustainer is controlled by or directly or indirectly linked to a constitutive promoter. In some embodiments, the immune initiator is controlled by or directly or indirectly linked to an constitutive promoter and the immune sustainer is controlled by or directly or indirectly linked to an inducible promoter.

In some embodiments both circuits may be integrated into the bacterial chromosome. In some embodiments, both circuits may be present on a plasmid. In some embodiments both circuits may be present on a plasmid. In some embodiments one circuit may be integrated into the bacterial chromosome and another circuit may be present on a plasmid.

In another embodiment, a bacterial strain expressing circuitry for immune initiation may be administered in conjunction with a separate bacterial strain expressing circuitry for immune sustenance. For example, one or more strain(s) of genetically engineered bacteria expressing immune initiatory circuitry and one or more separate strains of genetically engineered bacteria expressing immune sustainer circuitry may be administered sequentially, e.g., immune stimulator may be administered before immune sustainer. In another example, the immune initiator strain may be administered after the immune sustainer strain. In yet another example, the immune initiator strain may be administered concurrently with the immune sustainer strain.

Regardless of the sequence or timing of the administration (concurrent or sequential), engineered strains may express the circuitry for the immune sustainer sequentially or concurrently upon administration, i.e., timing and levels of expression are tuned using one or more mechanisms described herein, including but not limited to promoters and ribosome binding sites.

In a more specific example, one or more genetically engineered bacteria comprising gene sequence(s) encoding an enzyme for the production of a STING agonist and gene sequence(s) encoding an enzyme for the consumption of kynurenine can be provided in one strain comprising both circuits or in two or more strains, each comprising at least one of the circuits. In a non-limiting example of administration, an immune initiator producing strain is administered first, and then a immune sustainer producing strain is administered second. In a more specific non-limiting example of administration, a STING agonist producing strain is administered first, and then a kynurenine consuming strain is administered second.

Non-limiting examples of immune initiators and sustainers are described in Table 7 and Table 8.

TABLE 7
Immune Initiators

| Effect | Type | Effector |
|---|---|---|
| Immune activation/ | Cytokine/Chemokine | TNFα |
| Oncolysis/Priming | | |
| Immune activation/ | Cytokine/Chemokine | IFN-gamma |
| Priming | | |
| Immune activation/ | Cytokine/Chemokine | IFN-beta1 |
| Priming | | |
| Immune activation/ | Single chain | SIRPα |
| Phagocytosis/Priming | antibodies/Ligands | |
| Immune activation/ | Single chain | CD40L |
| Priming | antibodies/Ligands | |
| Immune activation/ | Metabolic conversion | STING agonist |
| Priming | | |
| Immune activation/ | Cytokine/Chemokine | GMCSF |
| Priming | | |
| Immune activation/ | T cell co-stimulatory | Agonistic anti-OX40 |
| Priming | receptor/Ligands | antibody or agonistic |
| | | OX40L |
| Immune activation/ | T cell co-stimulatory | Agonistic anti-41BB |
| Priming | receptor/Ligands | antibody or agonistic |
| | | 41BBL |
| Immune activation/ | T cell co-stimulatory | Agonistic anti-GITR |
| Priming | receptor/Ligands | antibody or agonistic |
| | | GITRL |
| Immune activation/ | Single chain | Anti-PD-1 antibody, |
| Priming | antibodies/Ligands | anti-PD-L1 antibody |
| | | (antagonistic) |
| Immune activation/ | Single chain | Anti-CTLA4 antibody |
| Priming | antibodies/Ligands | (antagonistic) |
| Oncolysis/Priming | Engineered | 5FC->5FU |
| | chemotherapy | |
| Oncolyis/Priming | Lytic peptides | Lytic peptides |
| | | (e.g. azurin) |
| Immune activation/ | Metabolic conversion | Arginine |
| Priming | | |

TABLE 8
Immune Sustainers

| Effect | Type | Effector |
|---|---|---|
| Immune Augmentation/ | Single chain | Anti-PD-1antibody, |
| Reversal of Exhaustion | antibodies/Ligands | anti-PD-L1 antibody |
| | | (antagonistic) |
| Immune Augmentation/ | Single chain | Anti-CTLA4 antibody |
| T cell Expansion | antibodies/Ligands | (antagonistic) |
| Immune Augmentation/ | Cytokine/Chemokine | IL-15 |
| T cell Expansion | | |
| Immune Augmentation/ | Cytokine/Chemokine | CXCL10 |
| T cell Recruitment | | |
| Immune Augmentation/ | Metabolic conversion | Arginine |
| T cell Expansion | | |
| Immune Augmentation/ | Metabolic conversion | Adenosine consumer |
| T cell Expansion | | |
| Immune Augmentation/ | Metabolic conversion | Kynurenine consumer |
| T cell Expansion | | |
| Immune Augmentation/ | Co-stimulatory | Agonistic anti-OX40 |
| T cell Expansion | Ligand/Receptor | antibody or OX40L |
| Immune Augmentation/ | Co-stimulatory | Agonistic anti-41BB |
| T cell Expansion | Ligand/Receptor | antibody or 41BBL |
| Immune Augmentation/ | Co-stimulatory | Agonistic anti-GITR |
| T cell Expansion | Ligand/Receptor | antibody or GITRL |
| Immune Augmentation/ | Cytokine/Chemokine | IL-12 |
| T cell Expansion | | |
| Antigen presentation/ | Cytokine/Chemokine | IFN-gamma |
| Tumor cell targeting | | |

In some combination embodiments, one or more effectors of Table 7 can be combined with one or more effectors of Table 8.

Multiple effectors can be selected which have an impact on different components of the immune response. Different immune response components which can be targeted by the effectors expressed by one or more genetically engineered bacteria include oncolysis, immune activation of APCs, and activation and priming of T cells (“immune initiator”), trafficking and infiltration, immune augmentation, T cell expansion, (“immune sustainer”). In some combination embodiments, an “immune initiator” is combined with an “immune sustainer”. In some embodiments, an immune initiator and/or an immune sustainer may further be combined with a stromal modulator, e.g., hyaluronidase. In some embodiments, two or more different bacteria comprising genes encoding an immune initiator and an immune sustainer, and optionally a stromal modulator may be combined and administered concurrently or sequentially.

In some embodiments, the genetically engineered bacteria are capable of producing effector or an immune modulator which initiates the immune response, i.e., an immune initiator. Non-limiting examples of such effectors for targeting immune activation and priming described herein include soluble SIRPα, anti-CD47 antibodies, and anti-CD40 antibodies, CD40-Ligand, TNFα, IFN-gamma, 5-FC to 5-FU conversion, and STING agonists. Non-limiting examples of effectors for targeting immune augmentation described herein include kynurenine degradation, adenosine degradation, arginine production, CXCL10, IL-15, IL-12 secretion, and checkpoint inhibition, e.g., through anti-PD-1 secretion or display. Non-limiting examples of effectors for targeting T cell expansion described herein include anti-PD-1 and anti-PD-L1 antibodies, anti-CTLA-4 antibodies, and IL-15.

In one embodiment, the immune initiator is not the same as the immune sustainer. As one non-limiting example, where the immune initiator is IFN-gamma, the immune sustainer is not IFN-gamma. In one embodiment, the immune initiator is different than the immune sustainer. As one non-limiting example, where the immune initiator is IFN-gamma, the immune sustainer is not IFN-gamma.

In one combination embodiment, genetically engineered bacteria comprise gene sequences for the production of one or more immune initiators combined with one or more gene sequences for the production of one or more immune sustainers. In alternate embodiments, the disclosure provides a composition comprising a combination (e.g., two or more) of different genetically engineered bacteria. In one such composition embodiment, one or more genetically engineered bacteria comprising gene sequences for the production of one or more immune initiators may be combined with one or more genetically engineered bacteria comprising gene sequences for the production of one or more immune sustainers. Alternatively, each bacteria in the composition may have both immune sustainer(s) and immune initiator(s).

In any of these combination and/or composition embodiments, one immune initiator may be a chemokine or cytokine. In some immune sustainer and immune initiator combination and/or composition embodiments, one immune initiator is a chemokine or cytokine and one immune sustainer is a single chain antibody. In some embodiments, one immune initiator is a chemokine or cytokine and one immune sustainer is a receptor ligand. In some embodiments, one immune initiator is a chemokine or cytokine and one immune sustainer is a receptor ligand. In some embodiments, one immune initiator is a chemokine or cytokine and one immune sustainer is a chemokine or cytokine. In some embodiments, one immune initiator is a chemokine or cytokine and one immune sustainer is a metabolic conversion. The metabolic conversion may be an arginine production, adenosine consumption, and/or kynurenine consumption. In some embodiments, the chemokine or cytokine initiator is selected from TNFα, IFN-gamma and IFN-beta1. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption.

In any of these combination and/or composition embodiments, one immune initiator may be a single chain antibody. In some immune sustainer and immune initiator combination and/or composition embodiments, one immune initiator is a single chain antibody and one immune sustainer is a single chain antibody. In some embodiments, one immune initiator is a single chain antibody and one immune sustainer is a receptor ligand. In some embodiments, one immune initiator is a single chain antibody and one immune sustainer is a receptor ligand. In some embodiments, one immune initiator is a single chain antibody and one immune sustainer is a chemokine or cytokine. In some embodiments, one immune initiator is a single chain antibody and one immune sustainer is a metabolic conversion. The metabolic conversion may be an arginine production, adenosine consumption, and/or kynurenine consumption. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption.

In any of these combination and/or composition embodiments, one immune initiator may be a receptor ligand. In some immune sustainer and immune initiator combination and/or composition embodiments, one immune initiator is a receptor ligand and one immune sustainer is a single chain antibody. In some embodiments, one immune initiator is a receptor ligand and one immune sustainer is a receptor ligand. In some embodiments, one immune initiator is a receptor ligand and one immune sustainer is a receptor ligand. In some embodiments, one immune initiator is a receptor ligand and one immune sustainer is a chemokine or cytokine. In some embodiments, one immune initiator is a receptor ligand and one immune sustainer is a metabolic conversion. The metabolic conversion may be an arginine production, adenosine consumption, and/or kynurenine consumption. In some embodiments, in which one immune initiator is a receptor ligand, the immune initiator is CD40L. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption. In some embodiments, the receptor ligand is SIRPα, or a fragment, variant or fusion protein thereof. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption.

In any of these combination and/or composition embodiments, one immune initiator may be a metabolic converter. In some immune sustainer and immune initiator combination and/or composition embodiments, one immune initiator is a metabolic conversion and one immune sustainer is a single chain antibody. In some embodiments, one immune initiator is a metabolic conversion and one immune sustainer is a receptor ligand. In some embodiments, one immune initiator is a metabolic conversion and one immune sustainer is a receptor ligand. In some embodiments, one immune initiator is a metabolic conversion and one immune sustainer is a chemokine or cytokine. In some embodiments, one immune initiator is a metabolic conversion and one immune sustainer is a metabolic conversion, e.g., selected from kynurenine consumer, tryptophan producer, arginine producer, and adenosine consumer. In some embodiments, the initiator metabolic conversion is a STING agonist producer, e.g., diadenylate cyclase, e.g., DacA. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption.

In any of these combination and/or composition embodiments, one immune initiator may be an engineered immunotherapy. In some immune sustainer and immune initiator combination and/or composition embodiments, one immune initiator is an engineered chemotherapy and one immune sustainer is a single chain antibody. In some embodiments, one immune initiator is an engineered chemotherapy and one immune sustainer is a receptor ligand. In some embodiments, one immune initiator is an engineered chemotherapy and one immune sustainer is a receptor ligand. In some embodiments, one immune initiator is an engineered chemotherapy and one immune sustainer is a chemokine or cytokine. In some embodiments, one immune initiator is an engineered chemotherapy and one immune sustainer is a metabolic conversion. The metabolic conversion may be an arginine production, adenosine consumption, and/or kynurenine consumption. In some embodiments, the initiator engineered chemotherapy is a 5FC to 5FU conversion, e.g., though codA, or variants or fusion proteins thereof. In any of these embodiments, the immune sustainer or augmenter may be selected from Anti-PD-1 single chain antibody, Anti-CTLA4 single chain antibody, IL-15, CXCL10 or a metabolic conversion. The metabolic conversion may be an arginine production, adenosine consumption, and/or kynurenine consumption.

In any of these combination and/or composition embodiments, one immune sustainer may be a single chain antibody. In some immune sustainer and immune initiator combination and/or composition embodiments, one immune sustainer is a single chain antibody and the immune initiator is a cytokine or chemokine. In some embodiments, one immune sustainer is a single chain antibody and the immune initiator is a receptor ligand. In some embodiments, one immune sustainer is a single chain antibody and the immune initiator is a single chain antibody. In some embodiments, one immune sustainer is a single chain antibody and the immune initiator is a metabolic conversion. In some embodiments, one immune sustainer is a single chain antibody and the immune initiator is an engineered chemotherapy. In some immune sustainer and immune initiator combination and/or composition embodiments, the immune sustainer is an anti-PD-1 antibody. In some immune sustainer and immune initiator combination and/or composition embodiments, the immune sustainer is an anti-CTLA4 antibody. In any of these embodiments, the immune initiator may be selected from TNFα, IFN-gamma, IFN-beta1, SIRPα, CD40L, STING agonist, and 5FC->5FU.

In any of these combination and/or composition embodiments, one immune sustainer may be a receptor ligand. In some immune sustainer and immune initiator combination and/or composition embodiments, one immune sustainer is a receptor ligand and the immune initiator is a cytokine or chemokine. In some embodiments, one immune sustainer is a receptor ligand and the immune initiator is a receptor ligand. In some embodiments, one immune sustainer is a receptor ligand and the immune initiator is a single chain antibody. In some embodiments, one immune sustainer is a receptor ligand and the immune initiator is a metabolic conversion. In some embodiments, one immune sustainer is a receptor ligand and the immune initiator is an engineered chemotherapy. In some immune sustainer and immune initiator combination and/or composition embodiments, the immune sustainer is PD1 or PDL1 or CTLA4, or a fragment, variant or fusion protein thereof. In any of these embodiments, the immune initiator may be selected from TNFα, IFN-gamma, IFN-beta1, SIRPα, CD40L, STING agonist, and 5FC->5FU.

In any of these combination and/or composition embodiments, one immune sustainer may be a cytokine or chemokine. In some immune sustainer and immune initiator combination and/or composition embodiments, one immune sustainer is a cytokine or chemokine and the immune initiator is a cytokine or chemokine. In some embodiments, one immune sustainer is a cytokine or chemokine and the immune initiator is a receptor ligand. In some embodiments, one immune sustainer is a cytokine or chemokine and the immune initiator is a single chain antibody. In some embodiments, one immune sustainer is a cytokine or chemokine and the immune initiator is a metabolic conversion. In some embodiments, one immune sustainer is a cytokine or chemokine and the immune initiator is an engineered chemotherapy. In some immune sustainer and immune initiator combination and/or composition embodiments, the immune sustainer is IL-15, or a fragment, variant or fusion protein thereof. In some immune sustainer and immune initiator combination and/or composition embodiments, the immune sustainer is CXCL10, or a fragment, variant or fusion protein thereof. In any of these embodiments, the immune initiator may be selected from TNFα, IFN-gamma, IFN-beta1, SIRPα, CD40L, STING agonist, and 5FC->5FU.

In any of these combination and/or composition embodiments, one immune sustainer may be a metabolic conversion. In some immune sustainer and immune initiator combination and/or composition embodiments, one immune sustainer is a metabolic conversion and the immune initiator is a cytokine or chemokine. In some embodiments, one immune sustainer is a metabolic conversion and the immune initiator is a receptor ligand. In some embodiments, one immune sustainer is a metabolic conversion and the immune initiator is a single chain antibody. In some embodiments, one immune sustainer is a metabolic conversion and the immune initiator is a metabolic conversion. In some embodiments, one immune sustainer is a metabolic conversion and the immune initiator is an engineered chemotherapy. In some immune sustainer and immune initiator combination and/or composition embodiments, the immune sustainer is kynurenine consumption. In some immune sustainer and immune initiator combination and/or composition embodiments, the immune sustainer is arginine production. In some immune sustainer and immune initiator combination and/or composition embodiments, the immune sustainer is adenosine consumption. In any of these embodiments, the immune initiator may be selected from TNFα, IFN-gamma, IFN-beta1, SIRPα, CD40L, STING agonist, and 5FC->5FU.

In any of these combination embodiments, the genetically engineered bacteria may comprise gene sequences encoding enzymes for the consumption of kynurenine (and optionally production of tryptophan) and gene sequences for the production of an immune initiator. In some embodiments, the genetically engineered bacteria comprise gene sequences encoding kynureninase and gene sequences for the production of an immune initiator. In some embodiments, the immune initiator combined with kynureninase is a chemokine or a cytokine. In some embodiments, the immune initiator combined with kynureninase is a single chain antibody. In some embodiments, the immune initiator combined with kynureninase is a receptor ligand. In some embodiments, the immune initiator combined with kynureninase is metabolic conversion, e.g., a STING agonist producer, e.g., diadenylate cyclase, e.g., dacA. In some embodiments, the immune initiator combined with kynureninase is an engineered chemotherapy, e.g., codA for the conversion of 5FC to 5FU. In some embodiments, the immune initiator is selected from TNFα, IFN-gamma, IFN-beta1, SIRPα, CD40L, STING agonist, and 5FC->5FU. In one embodiment, the genetically engineered bacteria comprise gene sequences encoding kynureninase and gene sequences encoding TNFα. In one embodiment, the genetically engineered bacteria comprise gene sequences encoding kynureninase and gene sequences encoding IFN-gamma. In one embodiment, the genetically engineered bacteria comprise gene sequences encoding kynureninase and gene sequences encoding IFN-beta1. In one embodiment, the genetically engineered bacteria comprise gene sequences encoding kynureninase and gene sequences encoding SIRPα or a variant thereof described herein. In one embodiment, the genetically engineered bacteria comprise gene sequences encoding kynureninase and gene sequences encoding CD40L. In one embodiment, the genetically engineered bacteria comprise gene sequences encoding kynureninase and gene sequences encoding an enzyme for the production of a STING agonist, e.g., dacA, for the production of cyclic-di-AMP. In one embodiment, the genetically engineered bacteria comprise gene sequences encoding kynureninase and gene sequences encoding an enzyme for the conversion of 5FC to 5FU, e.g., codA or a variant or fusion protein thereof. In any of these kynurenine consumption and immune initiator combination and/or composition embodiments, trpE may be deleted.

In any of these combination embodiments, the genetically engineered bacteria may comprise gene sequences encoding enzymes for the production of a STING agonist and gene sequences for the production of an immune sustainer. In some embodiments, the genetically engineered bacteria comprise gene sequences encoding e.g., diadenylate cyclase, e.g., dacA, and gene sequences for the production of an immune sustainer. In some embodiments, the immune sustainer combined with dacA is a chemokine or a cytokine. In some embodiments, the immune sustainer combined with dacA is a single chain antibody. In some embodiments, the immune sustainer combined with diadenylate cyclase, e.g., dacA is a receptor ligand. In some embodiments, the immune sustainer combined with diadenylate cyclase, e.g., dacA is metabolic conversion, e.g., an arginine producer, kynurenine consumer and/or adenosine consumer. In some embodiments, the immune sustainer is selected from anti-PD-1 antibody, anti-CTLA4 antibody, anti-PD-L1 antibody, IL-15, CXCL10, arginine producer, adenosine consumer, and kynurenine consumer. In one embodiment, the genetically engineered bacteria comprise gene sequences encoding dacA and gene sequences encoding an anti-PD-1 antibody. In one embodiment, the genetically engineered bacteria comprise gene sequences encoding diadenylate cyclase, e.g., dacA and gene sequences encoding anti-CTLA4 antibody. In one embodiment, the genetically engineered bacteria comprise gene sequences encoding dacA and gene sequences encoding IL-15. In one embodiment, the genetically engineered bacteria comprise gene sequences encoding diadenylate cyclase, e.g., dacA and gene sequences encoding CXCL10. In one embodiment, the genetically engineered bacteria comprise gene sequences encoding diadenylate cyclase, e.g., dacA and gene sequences encoding a circuitry for the production of arginine, e.g., as described herein. In one embodiment, the genetically engineered bacteria comprise gene sequences encoding diadenylate cyclase, e.g., dacA and gene sequences encoding an enzyme for the consumption of kynurenine, e.g., kynureninase, e.g., from Pseudomonas fluorescens. In one embodiment, the genetically engineered bacteria comprise gene sequences encoding diadenylate cyclase, e.g., dacA and gene sequences encoding an enzyme for the consumption adenosine, as described herein. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, dacA is from Listeria monocytogenes.

In any of these composition embodiments, one or more different genetically engineered bacteria comprising gene sequences encoding enzymes for the consumption of kynurenine (and optionally production of tryptophan) may be combined with one or more different genetically engineered bacteria comprising gene sequences for the production of an immune initiator. In some embodiments, the one or more different genetically engineered bacteria of the composition comprising gene sequences encoding kynureninase are combined with one or more different genetically engineered bacteria comprising gene sequences for the production of an immune initiator. In some embodiments, the immune initiator combined with kynureninase is a chemokine or a cytokine. In some embodiments, the immune initiator combined with kynureninase is a single chain antibody. In some embodiments, the immune initiator combined with kynureninase is a receptor ligand. In some embodiments, the immune initiator combined with kynureninase is metabolic conversion, e.g., a STING agonist producer, e.g., diadenylate cyclase, e.g., dacA. In some embodiments, the immune initiator combined with kynureninase is an engineered chemotherapy, e.g., codA for the conversion of 5FC to 5FU. In some embodiments, the immune initiator is selected from TNFα, IFN-gamma, IFN-beta1, SIRPα, CD40L, STING agonist, and 5FC->5FU. In one embodiment, the one or more different genetically engineered bacteria comprise gene sequences encoding kynureninase and gene sequences encoding TNFα. In one embodiment, the one or more different genetically engineered bacteria of the composition comprising gene sequences encoding kynureninase are combined with one or more different genetically engineered bacteria comprising gene sequences encoding IFN-gamma. In one embodiment, the one or more different genetically engineered bacteria of the composition comprising gene sequences encoding kynureninase are combined with one or more different genetically engineered bacteria comprising gene sequences encoding IFN-beta1. In one embodiment, the one or more different genetically engineered bacteria of the composition comprising gene sequences encoding kynureninase are combined with one or more different genetically engineered bacteria comprising gene sequences encoding SIRPα or a variant thereof described herein. In one embodiment, the one or more different genetically engineered bacteria of the composition comprising gene sequences encoding kynureninase are combined with one or more different genetically engineered bacteria comprising gene sequences encoding CD40L. In one embodiment, the one or more different genetically engineered bacteria of the composition comprising gene sequences encoding kynureninase are combined with one or more different genetically engineered bacteria comprising gene sequences encoding an enzyme for the production of a STING agonist, e.g., dacA for the production of cyclic-di-AMP. In one embodiment, the one or more different genetically engineered bacteria of the composition comprising gene sequences encoding kynureninase are combined with one or more different genetically engineered bacteria comprising gene sequences encoding an enzyme for the conversion of 5FC to 5FU, e.g., codA or a variant or fusion protein thereof. In any of these kynurenine consumption and immune initiator combination and/or composition embodiments, trpE may be deleted.

In any of these composition embodiments, the one or more different genetically engineered bacteria which may comprise gene sequences encoding enzymes for the production of a STING agonist may be combined with one or more different genetically engineered bacteria comprising gene sequences for the production of an immune sustainer. In some embodiments, the one or more different genetically engineered bacteria of the composition comprising gene sequences encoding diadenylate cyclase, e.g., dacA are combined with one or more different genetically engineered bacteria comprising gene sequences for the production of an immune sustainer. In some embodiments, the immune sustainer combined with diadenylate cyclase, e.g., dacA is a chemokine or a cytokine. In some embodiments, the immune sustainer combined with dacA is a single chain antibody. In some embodiments, the immune sustainer combined with diadenylate cyclase, e.g., dacA is a receptor ligand. In some embodiments, the immune sustainer combined with diadenylate cyclase, e.g., dacA is metabolic conversion, e.g., an arginine producer, kynurenine consumer and/or adenosine consumer. In some embodiments, the immune sustainer is selected from anti-PD-1 antibody, anti-CTLA4 antibody, IL-15, CXCL10, arginine producer, adenosine consumer, and kynurenine consumer. In one embodiment, the one or more different genetically engineered bacteria of the composition comprising gene sequences encoding diadenylate cyclase, e.g., dacA are combined with one or more different genetically engineered bacteria comprising gene sequences encoding an anti-PD-1 antibody. In one embodiment, the one or more different genetically engineered bacteria of the composition comprising gene sequences encoding diadenylate cyclase, e.g., dacA are combined with one or more different genetically engineered bacteria comprising gene sequences encoding anti-CTLA4 antibody. In one embodiment, the one or more different genetically engineered bacteria of the composition comprising gene sequences encoding diadenylate cyclase, e.g., dacA are combined with one or more different genetically engineered bacteria comprising gene sequences encoding IL-15. In one embodiment, the one or more different genetically engineered bacteria of the composition comprising gene sequences encoding diadenylate cyclase, e.g., dacA are combined with one or more different genetically engineered bacteria comprising gene sequences encoding CXCL10. In one embodiment, the one or more different genetically engineered bacteria of the composition comprising gene sequences encoding diadenylate cyclase, e.g., dacA are combined with one or more different genetically engineered bacteria comprising gene sequences encoding a circuitry for the production of arginine, e.g., as described herein. In one embodiment, the one or more different genetically engineered bacteria of the composition comprising gene sequences encoding diadenylate cyclase, e.g., dacA are combined with one or more different genetically engineered bacteria comprising gene sequences encoding an enzyme for the consumption of kynurenine, e.g., kynureninase, e.g., from Pseudomonas fluorescens. In one embodiment, the one or more different genetically engineered bacteria of the composition comprising gene sequences encoding dacA are combined with one or more different genetically engineered bacteria comprising gene sequences encoding an enzyme for the consumption adenosine, as described herein. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, dacA is from Listeria monocytogenes.

Any one or more immune initiator(s) may be combined with any one or more immune sustainer(s) in the cancer immunity cycle. Accordingly, in some embodiments, the genetically engineered bacteria are capable of producing one or more immune initiators which modulate, e.g., intensify, one or more of steps of the cancer immunity cycle (1) oncolysis, (2) activation of APCs and/or (3) priming and activation of T cells in combination with one or more immune sustainers, which modulate, e.g., boost, one or more of steps (4) T cell trafficking and infiltration, (5) recognition of cancer cells by T cells and/or T cell support and/or (6) the ability to overcome immune suppression. Non-limiting examples of immune initiators which modulate steps (1), (2), an (3) are provided herein. Non-limiting examples of immune sustainers which modulate steps (4), (5), an (6) are provided herein. Accordingly, any of these exemplary immune modulators may part of an immune initiator/immune sustainer combination which is capable of modulating one or more cancer immunity cycle steps as described herein. Accordingly, genetically engineered bacteria comprising gene sequences encoding combinations of immune initiator(s)/immune sustainer(s) can modulate combinations of cancer immunity cycle step, e.g., as follows: step (1), step (2), step (3), step (4), step (5), step (6); step (1), step (2), step (3), step (4), step (5); step (1), step (2), step (3), step (4), step (6); step (1), step (2), step (3), step (5), step (6); step (1), step (2), step (3), step (4); step (1), step (2), step (3), step (5); step (1), step (2), step (3), step (6); step (1), step (2), step (4), step (5), step (6); step (1), step (2), step (4), step (5); step (1), step (2), step (4), step (6); step (1), step (2), step (5), step (6); step (1), step (2), step (4); step (1), step (2), step (5); step (1), step (2), step (6); step (1), step (3), step (4), step (5), step (6); step (1), step (3), step (4), step (5); step (1), step (3), step (4), step (6); step (1), step (3), step (5), step (6); step (1), step (3), step (4); step (1), step (3), step (5); step (1), step (3), step (6); step (2), step (3), step (4), step (5), step (6); step (2), step (3), step (4), step (5); step (2), step (3), step (4), step (6); step (2), step (3), step (5), step (6); step (2), step (3), step (4); step (2), step (3), step (5); step (2), step (3), step (6); step (1), step (4), step (5), step (6); step (1), step (4), step (5); step (1), step (4), step (6); step (1), step (5), step (6); step (1), step (4); step (1), step (5); step (1), step (6); step (2), step (4), step (5), step (6); step (2), step (4), step (5); step (2), step (4), step (6); step (2), step (5), step (6); step (2), step (4); step (2), step (5); step (2), step (6); step (3), step (4), step (5), step (6); step (3), step (4), step (5); step (3), step (4), step (6); step (3), step (5), step (6); step (3), step (4); step (3), step (5); step (3), step (6).

In some embodiments, the genetically engineered bacteria of the invention produce the immune initiator and/or immune sustainer under low-oxygen conditions and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria of the invention produce the immune initiator and/or immune sustainer under the control of a constitutive promoter and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding immune initiators or immune sustainers may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding immune initiators or immune sustainers encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding immune initiators or immune sustainers immune initiators or immune sustainers encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In any of these embodiments and all combination embodiments, a engineered bacteria can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, oncolytic virus therapy, and blood product donation and transfusion. In any of these embodiments for producing an immune modulators, one or more engineered bacteria can be used in conjunction with other conventional immunotherapies used to treat cancer, such as checkpoint inhibitors, Fc-mediated ADCC, BiTE, TCR, adoptive cell therapy (TILs, CARs, NK/NKT, etc.), and any of the other immunotherapies described herein and otherwise known in the art. In any of these embodiments, the engineered bacteria can be used in conjunction with a cancer or tumor vaccine.

### Combinations of Immune Initiators and Immune Initiators

In some embodiments, the genetically engineered bacteria are capable of producing two or more initiators which modulate, e.g., intensify, one or more of steps (1), (2), and/or (3). Alternatively, the disclosure provides a composition comprising a combination (e.g., two or more) of different genetically engineered bacteria, each bacteria encoding one or more immune initiators. In yet another embodiment, the disclosure provides for the administration of an immune initiator, in combination with, e.g., before, at the same time as, or after, a modified microorganism capable of producing an immune initiator. Such distinct or different combinations and/or bacterial strains can be administered concurrently or sequentially. Regardless of the sequence or timing of the administration (concurrent or sequential), engineered strains may express the circuitry for the immune sustainer sequentially or concurrently upon administration, i.e., timing and levels of expression are tuned using one or more mechanisms described herein, including but not limited to promoters and ribosome binding sites.

In some embodiments of the disclosure, in which a microorganism genetically engineered to express two or more immune initiator circuits, the microorganism first produces higher levels of a first immune stimulator and at a later time point a second immune initiator. In some embodiments of the disclosure, in which a microorganism genetically engineered to express two or more immune initiator circuits, the microorganism produces a first immune stimulator and a second immune initiator concurrently. In certain embodiments, the one or more gene sequences are under the control of inducible promoters known in the art or described herein. For example, such inducible promoters may be induced under low-oxygen conditions, such as an FNR promoter. In some embodiments, the one or more gene sequence(s) encoding the immune initiators are operably linked to a directly or indirectly inducible promoter that is induced under inflammatory conditions (e.g., RNS, ROS), as described herein. In other embodiments, the promoters are induced in the presence of certain molecules or metabolites, e.g., in the presence of molecules or metabolites associated with the tumor microenvironment and/or with immune suppression. In some embodiments, the promoters are induced in certain tissue types. In some embodiments, promoters are induced in the presence of certain gut-specific or tumor-specific molecules or metabolites. In some embodiments, the promoters are induced in the presence of some other metabolite that may or may not be present in the gut or the tumor, such as arabinose, cumate, and salicylate or another chemical or nutritional inducer known in the art or described herein. In certain embodiments, the two or more immune initiator circuits are under the control of constitutive promoters described herein or known in the art, e.g., whose expression can be fine-tuned using ribosome binding sites of different strengths. Such microorganisms optionally also comprise an auxotrophic modification, e.g., an auxotrophic modification in amino acid or nucleotide metabolism. Non-limiting examples include ΔDapA or ΔThyA or both.

Promoters controlling expression of the two or more immune initiators may be the same or different and may be induced by the same chemical or environmental inducer or a different chemical or environmental inducers. First inducer and second inducer may be administered sequentially or concurrently. In some embodiments, one initiator is under the control of a low oxygen promoter. In some embodiments, both initiators are under control of a low oxygen promoter. In some embodiments, one initiator is under control of a constitutive promoter. In some embodiments, both initiators are under control of a constitutive promoter.

In some embodiments both circuits may be integrated into the bacterial chromosome. In some embodiments, both circuits may be present on a plasmid. In some embodiments both circuits may be present on a plasmid. In some embodiments one circuit may be integrated into the bacterial chromosome and another circuit may be present on a plasmid.

Any immune initiator may be combined with one or more additional same or different immune initiator(s), which modulate the same or a different step in the cancer immunity cycle.

In some embodiments, the genetically engineered bacteria are capable of producing one or more immune initiators, which modulate, e.g., intensify, one or more of steps (1) Lysis of tumor cells, (2), activation of APCs and/or (3) priming and activation of T cells. In some embodiments, the genetically engineered bacteria are capable of producing two or more initiators which modulate, e.g., intensify, one or more of steps (1), (2), and/or (3). In some embodiments, the genetically engineered bacteria produce two or more immune initiators, which modulate, e.g., intensify, the same step of the cancer immunity cycle. In one example, the genetically engineered bacteria produce two or more immune initiators which modulate oncolysis (step (1)). In one example, the genetically engineered bacteria produce two or more immune initiators which modulate activation of APCs (step (2)). In one example, the genetically engineered bacteria produce two or more immune initiators which modulate, e.g., enhance, priming and activation of T cells (step (3)). In some embodiments, the genetically engineered bacteria produce two or more immune initiators, which modulate, e.g., intensify, the same step. In a non-limiting example, the genetically engineered bacteria produce one or more immune initiators which modulate, e.g., intensify oncolysis (step (1)) and one or more immune initiators which modulate, e.g., intensify activation of APCs (step (2)). In another non-limiting example, the genetically engineered bacteria produce one or more immune initiators which modulate, e.g., intensify oncolysis (step (1)) and one or more immune initiators which modulate, e.g., intensify priming and activation of T cells (step (3)). In another non-limiting example, the genetically engineered bacteria produce one or more immune initiators which modulate, e.g., intensify activation of APCs (step (2)) and one or more immune initiators which modulate, e.g., intensify priming and activation of T cells (step (3)). In yet another non-limiting example, the genetically engineered bacteria produce one or more immune initiators which modulate, e.g., intensify step (1) Oncolysis, one or more immune initiators which modulate, e.g., intensify activation of APCs (step (2)), and one or more immune initiators which modulate, e.g., intensify priming and activation of T cells (step (3)).

In some embodiments, the genetically engineered bacteria comprise gene circuitry for the production of or more immune initiators, which modulate, e.g., intensify, one or more of steps (1) oncolysis, (2) activation of APCs and/or (3) priming and activation of T cells. In some embodiments, the genetically engineered bacteria comprise one or more genes encoding one or more immune initiators, which modulate, e.g., intensify, one or more of steps (1) oncolysis, (2) activation of APCs and/or (3) priming and activation of T cells. Any immune initiator may be combined with another immune initiator, which modulates the same or a different step. In some embodiments, the genetically engineered bacteria comprise gene sequence(s) encoding two or more initiators which modulate, e.g., intensify, one or more of steps (1) oncolysis, (2) activation of APCs, and/or (3) priming and activation of T cells. In some embodiments, the genetically engineered bacteria comprise gene sequence(s) encoding two or more immune initiators, which modulate, e.g., intensify, the same step. In one example, the genetically engineered bacteria comprise gene sequence(s) encoding two or more immune initiators which modulate step (1) lysis of tumor cells (oncolysis). In one example, the genetically engineered bacteria comprise gene sequence(s) encoding two or more immune initiators which modulate activation of APCs (step (2)). In one example, the genetically engineered bacteria comprise gene sequence(s) encoding two or more immune initiators which modulate priming and activation of T cells (step (3)). In some embodiments, the genetically engineered bacteria comprise gene sequence(s) encoding two or more immune initiators, which modulate, e.g., intensify, the same step. In a non-limiting example, the genetically engineered bacteria comprise gene sequence(s) encoding one or more immune initiators which modulate, e.g., intensify, oncolysis (step (1)) and one or more immune initiators which modulate, e.g., intensify activation of APCs (step (2)). In another non-limiting example, the genetically engineered bacteria comprise gene sequence(s) encoding one or more immune initiators which modulate, e.g., intensify, oncolysis (step (1)) and one or more immune initiators which modulate, e.g., intensify, priming and activation of T cells (step (3)). In another non-limiting example, the genetically engineered bacteria comprise gene sequence(s) encoding one or more immune initiators which modulate, e.g., intensify, activation of APCs (step (2)) and one or more immune initiators which modulate, e.g., intensify, priming and activation of T cells (step (3)). In yet another non-limiting example, the genetically engineered bacteria comprise gene sequence(s) encoding one or more immune initiators which modulate, e.g., intensify, oncolysis (step (1)), one or more immune initiators which modulate, e.g., intensify, activation of APCs (step (2)), and one or more immune initiators which modulate, e.g., intensify, priming and activation of T cells (step (3)).

In some embodiments, the genetically engineered bacteria of the invention produce two or more immune initiators under low-oxygen conditions and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria of the invention produce the two or more immune initiators under the control of a constitutive promoter and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In any of these embodiments and all combination embodiments, a engineered bacteria can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, oncolytic virus therapy, and blood product donation and transfusion.

The circuit encoding the immune initiators may be under the control of a constitutive or inducible promoter, e.g., low oxygen inducible promoter or any other constitutive or inducible promoter described herein.

In any of these embodiments, the gene sequence(s) encoding the immune initiators may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding the immune initiators encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding the immune initiators encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In any of these embodiments for producing an immune modulator, one or more engineered bacteria can be used in conjunction with other conventional immunotherapies used to treat cancer, such as checkpoint inhibitors, Fc-mediated ADCC, BiTE, TCR, adoptive cell therapy (TILs, CARs, NK/NKT, etc.), and any of the other immunotherapies described herein and otherwise known in the art. In any of these embodiments, the engineered bacteria can be used in conjunction with a cancer or tumor vaccine.

### Combinations of Immune Sustainers and Immune Sustainers

In some embodiments, the genetically engineered bacteria are capable of producing two or more sustainers which modulate, e.g., intensify, one or more of steps (1), (2), and/or (3). Alternatively, the disclosure provides a composition comprising a combination (e.g., two or more) of different genetically engineered bacteria, each bacteria encoding one or more immune sustainers. In yet another embodiment, the disclosure provides for the administration of an immune sustainer in combination with, e.g., before, at the same time as, or after, a modified microorganism capable of producing an immune sisatomer. Such distinct or different combinations or bacterial strains can be administered concurrently or sequentially. Regardless of the sequence or timing of the administration (concurrent or sequential), engineered strains may express the circuitry for the immune sustainer sequentially or concurrently upon administration, i.e., timing and levels of expression are tuned using one or more mechanisms described herein, including but not limited to promoters and ribosome binding sites.

In some embodiments of the disclosure, in which a microorganism genetically engineered to express two or more immune sustainer circuits, the microorganism first produces higher levels of a first immune stimulator and at a later time point a second immune sustainer. In some embodiments of the disclosure, in which a microorganism genetically engineered to express two or more immune sustainer circuits, the microorganism produces a first immune stimulator and a second immune sustainer concurrently. In certain embodiments, the one or more gene sequences are under the control of inducible promoters known in the art or described herein. For example, such inducible promoters may be induced under low-oxygen conditions, such as an FNR promoter. In some embodiments, the one or more gene sequence(s) encoding the immune sustainers are operably linked to a directly or indirectly inducible promoter that is induced under inflammatory conditions (e.g., RNS, ROS), as described herein. In other embodiments, the promoters are induced in the presence of certain molecules or metabolites, e.g., in the presence of molecules or metabolites associated with the tumor microenvironment and/or with immune suppression. In some embodiments, the promoters are induced in certain tissue types. In some embodiments, promoters are induced in the presence of certain gut-specific or tumor-specific molecules or metabolites. In some embodiments, the promoters are induced in the presence of some other metabolite that may or may not be present in the gut or the tumor, such as arabinose, cumate, and salicylate or another chemical or nutritional inducer known in the art or described herein. In certain embodiments, the two or more immune sustainer circuits are under the control of constitutive promoters described herein or known in the art, e.g., whose expression can be fine-tuned using ribosome binding sites of different strengths. Such microorganisms optionally also comprise an auxotrophic modification, e.g., an auxotrophic modification in amino acid aor a nucleotide metabolism, such as ΔDapA or ΔThyA or both.

Promoters controlling expression of the two or more immune sustainers may be the same or different and may be induced by the same chemical or environmental inducer or a different chemical or environmental inducers. First inducer and second inducer may be administered sequentially or concurrently. In some embodiments, one sustainer is under the control of a low oxygen promoter. In some embodiments, both sustainers are under control of a low oxygen promoter. In some embodiments, one sustainer is under control of a constitutive promoter. In some embodiments, both sustainers are under control of a constitutive promoter.

In some embodiments both circuits may be integrated into the bacterial chromosome. In some embodiments, both circuits may be present on a plasmid. In some embodiments both circuits may be present on a plasmid. In some embodiments one circuit may be integrated into the bacterial chromosome and another circuit may be present on a plasmid.

In some embodiments, the genetically engineered bacteria are capable of producing one or more immune sustainers, which modulate, e.g., boost, one or more of steps (4) T cell trafficking and infiltration, (5) recognition of cancer cells by T cells and/or T cell support and/or (6) the ability to overcome immune suppression. Any immune sustainer may be combined with another immune sustainer, which modulates the same or a different step. In some embodiments, the genetically engineered bacteria are capable of producing two or more sustainers which modulate, e.g., boost, one or more of steps (4), (5), and/or (6). In some embodiments, the genetically engineered bacteria produce two or more immune sustainers, which modulate, e.g., boost, the same step. In one example, the genetically engineered bacteria produce two or more immune sustainers which modulate T cell trafficking and infiltration (step (4)). In one example, the genetically engineered bacteria produce two or more immune sustainers which modulate step (5) recognition of cancer cells by T cells and/or T cell support. In one example, the genetically engineered bacteria produce two or more immune sustainers which modulate, e.g., enhance, the ability to overcome immune suppression (step (6)). In some embodiments, the genetically engineered bacteria produce two or more immune sustainers, which modulate, e.g., boost, the same step. In a non-limiting example, the genetically engineered bacteria produce one or more immune sustainers which modulate, e.g., boost, T cell trafficking and infiltration (step (4)) and one or more immune sustainers which modulate, e.g., boost recognition of cancer cells by T cells and/or T cell support (step (5)). In another non-limiting example, the genetically engineered bacteria produce one or more immune sustainers which modulate, e.g., boost, T cell trafficking and infiltration (step (4)) and one or more immune sustainers which modulate, e.g., boost, the ability to overcome immune suppression (step (6)). In another non-limiting example, the genetically engineered bacteria produce one or more immune sustainers which modulate, e.g., boost recognition of cancer cells by T cells and/or T cell support (step (5)) and one or more immune sustainers which modulate, e.g., boost the ability to overcome immune suppression (step (6)). In yet another non-limiting example, the genetically engineered bacteria produce one or more immune sustainers which modulate, e.g., boost, step (4) T cell trafficking and infiltration, one or more immune sustainers which modulate, e.g., boost, recognition of cancer cells by T cells and/or T cell support (step (5)), and one or more immune sustainers which modulate, e.g., boost the ability to overcome immune suppression (step (6)).

In some embodiments, the genetically engineered bacteria comprise gen circuitry for the production of one or more immune sustainers, which modulate, e.g., boost, one or more of steps (4) T cell trafficking and infiltration, (5) recognition of cancer cells by T cells and/or T cell support and/or (6) the ability to overcome immune suppression. In some embodiments, the genetically engineered bacteria comprise one or more genes encoding one or more immune sustainers, which modulate, e.g., boost, one or more of steps (4) T cell trafficking and infiltration, (5) recognition of cancer cells by T cells and/or T cell support and/or (6) the ability to overcome immune suppression. In some embodiments, the genetically engineered bacteria comprise gene sequence(s) encoding two or more sustainers which modulate, e.g., boost, one or more of steps (4) T cell trafficking and infiltration, (5) recognition of cancer cells by T cells and/or T cell support, and/or (6) the ability to overcome immune suppression. In some embodiments, the genetically engineered bacteria comprise gene sequence(s) encoding two or more immune sustainers, which modulate, e.g., boost, the same step. In one example, the genetically engineered bacteria comprise gene sequence(s) encoding two or more immune sustainers which modulate T cell trafficking and infiltration (step (4)). In one example, the genetically engineered bacteria comprise gene sequence(s) encoding two or more immune sustainers which modulate recognition of cancer cells by T cells and/or T cell support (step (5)). In one example, the genetically engineered bacteria comprise gene sequence(s) encoding two or more immune sustainers which modulate the ability to overcome immune suppression (step (6)). In some embodiments, the genetically engineered bacteria comprise gene sequence(s) encoding two or more immune sustainers, which modulate, e.g., boost, the same step. In a non-limiting example, the genetically engineered bacteria comprise gene sequence(s) encoding one or more immune sustainers which modulate, e.g., boost, T cell trafficking and infiltration (step (4)) and one or more immune sustainers which modulate, e.g., boost recognition of cancer cells by T cells and/or T cell support (step (5)). In another non-limiting example, the genetically engineered bacteria comprise gene sequence(s) encoding one or more immune sustainers which modulate, e.g., boost, T cell trafficking and infiltration (step (4)) and one or more immune sustainers which modulate, e.g., boost, the ability to overcome immune suppression (step (6)). In another non-limiting example, the genetically engineered bacteria comprise gene sequence(s) encoding one or more immune sustainers which modulate, e.g., boost, recognition of cancer cells by T cells and/or T cell support (step (5)) and one or more immune sustainers which modulate, e.g., boost, the ability to overcome immune suppression (step (6)). In yet another non-limiting example, the genetically engineered bacteria comprise gene sequence(s) encoding one or more immune sustainers which modulate, e.g., boost, T cell trafficking and infiltration (step (4)), one or more immune sustainers which modulate, e.g., boost, recognition of cancer cells by T cells and/or T cell support (step (5)), and one or more immune sustainers which modulate, e.g., boost, the ability to overcome immune suppression (step (6)).

In some embodiments, the genetically engineered bacteria of the invention produce the two or more immune sustainers under low-oxygen conditions and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria of the invention produce the two or more immune sustainers under the control of a constitutive promoter and are capable of reducing cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

The circuit encoding the immune sustainers may be under the control of a constitutive or inducible promoter, e.g., low oxygen inducible promoter or any other constitutive or inducible promoter described herein.

In any of these embodiments, the gene sequence(s) encoding the immune sustainers may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding the immune sustainers encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding the immune sustainers encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

In any of these embodiments and all combination embodiments, a engineered bacteria can be used in conjunction with conventional cancer therapies, such as surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, oncolytic virus therapy, and blood product donation and transfusion. In any of these embodiments for producing an immune modulator, one or more engineered bacteria can be used in conjunction with other conventional immunotherapies used to treat cancer, such as checkpoint inhibitors, Fc-mediated ADCC, BiTE, TCR, adoptive cell therapy (TILs, CARs, NK/NKT, etc.), and any of the other immunotherapies described herein and otherwise known in the art. In any of these embodiments, the engineered bacteria can be used in conjunction with a cancer or tumor vaccine.

### STING Agonist Combinations

### STING Agonists and Kynurenine Consumption

In one embodiment, the genetically engineered bacteria comprise one or more genes encoding enzymes for the production of STING agonist(s) in combination with one or more genes encoding enzymes for the consumption of kynurenine. In one embodiment, the STING agonist produced is c-di-AMP. In one embodiment, the genetically engineered bacteria comprises gene sequences encoding a diadenylate cyclase in combination with gene sequences encoding a kynureninase. In one embodiment, the diadenylate cyclase is from Listeria monocytogenes. In one embodiment, the kynureninase is from Pseudomonas fluorescens. In one specific embodiment, the diadenylate cyclase is from Listeria monocytogenes and the kynureninase is from Pseudomonas fluorescens. In one embodiment, the STING agonist produced is cyclic-di-GAMP. In one embodiment, the genetically engineered bacteria comprises gene sequences encoding a c-di-GAMP synthase in combination with gene sequences encoding a kynureninase. In one embodiment, the c-di-GAMP synthases is from Vibrio cholerae. In some embodiments, the cGAS is the human cGAS. In one embodiment, the kynureninase is from Pseudomonas fluorescens. In one specific embodiment, the c-di-GAMP synthase is from Vibrio cholerae and the kynureninase is from Pseudomonas fluorescens. In one specific embodiment, the c-di-GAMP synthase is human cGAS and the kynureninase is from Pseudomonas fluorescens. In one specific embodiment, the kynureninase from Pseudomonas fluorescens is chromosomally integrated and under control of a constitutive promoter.

In some embodiments, the microorganism of the disclosure is genetically engineered to express gene sequence(s) encoding one or more enzymes for the production of a STING agonist and additionally one or more gene sequence(s) for the expression of a kynurenine consuming enzyme. Non limiting examples of such enzymes for the production of STING agonists include dacA, e.g., from Listeria monocytogenes. Non-limiting examples of such kynurenine consuming enzymes include kynureninase (e.g., kynureninase from Pseudomonas fluorescens). More generally, immune initiator circuits (STING agonist producer or others described herein) may be combined with immune sustainer circuits (e.g., kynurenine consumption or others described herein).

In some embodiments of the disclosure, in which a microorganism genetically engineered to express an STING agonist and a kynurenine consumption circuit, the microorganism first produces higher levels of STING agonist producing enzyme e.g., DacA, e.g., from Listeria monocytogenes and at a later time point produces kynureninase, e.g., from Pseudomonas fluorescens). In some embodiments, expression of the STING agonist producing enzyme, e.g., dacA, is induced by an inducer. In some embodiments, kynureninase) is induced by an inducer. In some embodiments, STING agonist producing enzyme, e.g., dacA, and kynureninase are induced by one or more inducer(s). A first inducer (e.g., inducing dacA expression) and a second inducer (e.g., inducing kynureninase expression) may be the same or different inducers. First and second inducer may be administered sequentially or concurrently. Non-limiting examples of inducers include conditions of the gut or tumor microenvironment (e.g., low oxygen, certain nutrients, etc.), certain in vitro conditions during cell culture, or chemical inducers (e.g., arabinose, cumate, and salicylate, IPTG or other chemical inducers described herein), which can be added in vitro or in vivo. In other embodiments, both STING agonist producing enzyme, e.g., dacA) and kynureninase) are controlled by or directly or indirectly linked to constitutive promoters, including but not limited to those described herein. In some embodiments, the STING agonist producing enzyme, e.g., dacA) is controlled by or directly or indirectly linked to an inducible promoter and the kynureninase is controlled by or directly or indirectly linked to a constitutive promoter. In some embodiments, the STING agonist producing enzyme, e.g., dacA is controlled by or directly or indirectly linked to an constitutive promoter and the kynureninase is controlled by or directly or indirectly linked to an inducible promoter. In some embodiments, both circuits may be integrated into the bacterial chromosome. In some embodiments, both circuits may be present on a plasmid. In some embodiments, both circuits may be present on a plasmid. In some embodiments, one circuit may be integrated into the bacterial chromosome and another circuit may be present on a plasmid.

In yet another embodiment, one or more strain(s) of genetically engineered bacteria expressing STING agonist producing circuitry, e.g., dacA, and one or more separate strain(s) genetically engineered bacteria expressing kynurenine consumption circuitry (e.g., kynureninase) may be administered sequentially, e.g., STING agonist producer may be administered before kynurenine consumer. In other embodiments, STING agonist producer may be administered after kynurenine consumer. In yet another embodiment, STING agonist producer may be administered concurrently with the kynurenine consumer.

In alternate embodiments, the disclosure provides a composition comprising a combination (e.g., two or more) of different genetically engineered bacteria, each bacteria encoding a different immune modulator. In one embodiment, the composition comprises genetically engineered bacteria comprising one or more genes encoding enzymes for the production of STING agonist(s) and genetically engineered bacteria comprising one or more genes encoding enzymes for the consumption of kynurenine. In one embodiment, the STING agonist produced is c-di-AMP. In one embodiment, the composition comprises genetically engineered bacteria comprising gene sequences encoding a diadenylate cyclase and genetically engineered bacteria comprising gene sequences encoding a kynureninase. In one embodiment, the diadenylate cyclase is from Listeria monocytogenes. In one embodiment, the kynureninase is from Pseudomonas fluorescens. In one specific embodiment, the diadenylate cyclase is from Listeria monocytogenes and the kynureninase is from Pseudomonas fluorescens. In one embodiment, the STING agonist produced is cyclic-di-GAMP. In one embodiment, the composition comprises genetically engineered bacteria comprising gene sequences encoding a c-di-GAMP synthase and genetically engineered bacteria comprising gene sequences encoding a kynureninase. In one embodiment, the c-di-GAMP synthases is from Vibrio cholerae. In one specific embodiment, the c-di-GAMP synthase is from Vibrio cholerae and the kynureninase is from Pseudomonas fluorescens. IN some embodiments, the genetically engineered bacteria comprise sequences for the production of human cGAS. In one embodiment, the kynureninase is from Pseudomonas fluorescens. In one specific embodiment, the c-di-GAMP synthase is human cGAS and the kynureninase is from Pseudomonas fluorescens. In one specific embodiment, the kynureninase from Pseudomonas fluorescens is chromosomally integrated and under control of a constitutive promoter.

In any of these STING agonist production and kynurenine consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In any of these STING agonist production and kynurenine consumption embodiments, the genetically engineered bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more kynurenine than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more STING agonist, e.g., cyclic-di-AMP, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more STING agonist, e.g., cyclic-di-AMP, than unmodified bacteria of the same bacterial subtype under the same conditions.

In yet another embodiment, the genetically engineered bacteria consume 1.0-1.2-fold, 1.2-1.4-fold, 1.4-1.6-fold, 1.6-1.8-fold, 1.8-2-fold, or two-fold more kynurenine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more kynurenine than unmodified bacteria of the same bacterial subtype under the same conditions.

In any of these STING agonist production and kynurenine consumption embodiments, the genetically engineered bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more ATP than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ATP than unmodified bacteria of the same bacterial subtype under the same conditions.

In a non-limiting example lowering of kynurenine levels in the block may be measured as an indicator for kynureninase expressing strain activity.

In any of these STING agonist production and kynurenine consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more tryptophan than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more tryptophan than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more tryptophan than unmodified bacteria of the same bacterial subtype under the same conditions.

In any of these embodiments, the genetically engineered bacteria increase the kynurenine consumption rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the kynurenine consumption rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase the kynurenine consumption rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one embodiment, the genetically engineered bacteria increase the kynurenine consumption by about 80% to 100% relative to unmodified bacteria of the same bacterial subtype under the same conditions, after 4 hours. In one embodiment, the genetically engineered bacteria increase the kynurenine consumption by about 90% to 100% relative to unmodified bacteria of the same bacterial subtype under the same conditions after 4 hours. In one specific embodiment, the genetically engineered bacteria increase the kynurenine consumption by about 95% to 100% relative to unmodified bacteria of the same bacterial subtype under the same conditions, after 4 hours. In one specific embodiment, the genetically engineered bacteria increase the kynurenine consumption by about 99% to 100% relative to unmodified bacteria of the same bacterial subtype under the same conditions, after 4 hours. In yet another embodiment, the genetically engineered bacteria increase the kynurenine consumption by about 10-50 fold after 4 hours. In yet another embodiment, the genetically engineered bacteria increase the kynurenine consumption by about 50-100 fold after 4 hours. In yet another embodiment, the genetically engineered bacteria increase the kynurenine consumption by about 100-500 fold after 4 hours. In yet another embodiment, the genetically engineered bacteria increase the kynurenine consumption by about 500-1000 fold after 4 hours. In yet another embodiment, the genetically engineered bacteria increase the kynurenine consumption by about 1000-5000 fold after 4 hours. In yet another embodiment, the genetically engineered bacteria increase the kynurenine consumption by about 5000-10000 fold after 4 hours. In yet another embodiment, the genetically engineered bacteria increase the kynurenine consumption by about 10000-1000 fold after 4 hours.

In any of these embodiments, the genetically engineered bacteria increase STING agonist production rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the STING agonist production rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase STING agonist production rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one embodiment, the genetically engineered bacteria increase STING agonist production by about 80% to 100% relative to unmodified bacteria of the same bacterial subtype under the same conditions, after 4 hours. In one embodiment, the genetically engineered bacteria increase STING agonist production by about 90% to 100% relative to unmodified bacteria of the same bacterial subtype under the same conditions after 4 hours. In one specific embodiment, the genetically engineered bacteria increase STING agonist production by about 95% to 100% relative to unmodified bacteria of the same bacterial subtype under the same conditions, after 4 hours. In one specific embodiment, the genetically engineered bacteria increase the STING agonist production by about 99% to 100% relative to unmodified bacteria of the same bacterial subtype under the same conditions, after 4 hours. In yet another embodiment, the genetically engineered bacteria increase the STING agonist production by about 10-50 fold after 4 hours. In yet another embodiment, the genetically engineered bacteria increase STING agonist production by about 50-100 fold after 4 hours. In yet another embodiment, the genetically engineered bacteria increase STING agonist production by about 100-500 fold after 4 hours. In yet another embodiment, the genetically engineered bacteria increase STING agonist production by about 500-1000 fold after 4 hours. In yet another embodiment, the genetically engineered bacteria increase the STING agonist production by about 1000-5000 fold after 4 hours. In yet another embodiment, the genetically engineered bacteria increase the STING agonist production by about 5000-10000 fold after 4 hours. In yet another embodiment, the genetically engineered bacteria increase STING agonist production by about 10000-1000 fold after 4 hours.

In any of these STING agonist production and kynurenine consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and kynurenine consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and kynurenine consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and kynurenine consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and kynurenine consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some STING agonist production and kynurenine consumption embodiments, the genetically engineered microorganisms are capable of expressing any one or more of the described circuits for the degradation of adenosine and kynurenine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the STING agonist production and the degradation of kynurenine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these STING agonist production and kynurenine consumption embodiments, any one or more of the described STING agonist production circuits and kynurenine consumption circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits.

In any of these embodiments, the one or more bacteria genetically engineered to produce STING agonists and consume kynurenine may be administered alone or in combination with one or more immune checkpoint inhibitors described herein, including but not limited to anti-CTLA4, anti-PD1, or anti-PD-L1 antibodies. In any of these embodiments, the one or more bacteria genetically engineered to produce STING agonists and consume kynurenine may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered to produce STING agonists and consume kynurenine may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to produce STING agonists and consume kynurenine may be genetically engineered to produce and secrete or display on their surface one or more immune stimulatory agonists described herein, e.g., agonistic antibodies, including but not limited to anti-OX40, anti-41BB, or anti-GITR antibodies.

In certain embodiments, one or more genetically engineered bacteria expressing any one or more of the described circuits for the production of one or more STING agonists and one or more kynureninase(s), e.g., from Pseudomonas fluorescens, may be genetically engineered to produce and secrete or display on their surface one or more immune checkpoint inhibitors described herein, including but not limited to anti-CTLA4, anti-PD1, or anti-PD-L1 antibodies. In one embodiment, one or more genetically engineered bacteria comprise gene sequence(s) encoding DacA, e.g., from Listeria monocytogenes, wherein DacA is operably linked to a promoter inducible under low oxygen conditions, e.g., a FNR promoter. The bacteria comprising gene sequences encoding dacA comprise a mutation or deletion in dapA. The one or more genetically engineered bacteria further comprise gene sequences encoding kynureninase from Pseudomonas fluorescens and the bacterium comprising gene sequences encoding kynureninase further comprises a mutation or deletion in TrpE. In certain embodiments, dacA and kynureninase sequences are integrated into the bacterial chromosome. In one specific embodiment, the one or more genetically engineered bacteria may further comprise mutation(s) or deletion(s) in ThyA. In one specific embodiment, the checkpoint inhibitor is PD-1. In one specific embodiment, the checkpoint inhibitor is PD-L1. In one specific embodiment, the checkpoint inhibitor is CTLA-4.

In one embodiment, dacA circuitry (e.g., from Listeria monocytogenes, e.g., under control of a low oxygen promoter and chromosomally integrated), kynureninase circuitry (e.g., from Pseudomonas fluorescens, e.g., under the control of a constitutive promoter and chromosomally integrated) and auxotrophic mutations (mutations or deletions in TrpE, dapA, and ThyA), and checkpoint inhibitor secretion or display circuitry (e.g., under the control of a constitutive promoter or inducible promoter and chromosomally integrated) are combined in one bacterium. In an alternate embodiment, a bacterial composition comprises a first bacterium comprising gene sequences encoding dacA (e.g., from Listeria monocytogenes, e.g., under control of a low oxygen promoter and chromosomally integrated), further comprising a mutation or deletion in DapA, and with an optional mutations or deletions in ThyA and a second bacterium comprising gene sequences encoding kynureninase circuitry (e.g., from Pseudomonas fluorescens, e.g., under the control of a constitutive promoter and chromosomally integrated), a mutation or deletion in TrpE, and optionally a mutation or deletion in ThyA. The composition further comprises a third bacterium engineered to secrete or display the checkpoint inhibitor. Alternatively the first or second bacterium are engineered to secrete or display the checkpoint inhibitor.

In any of these combination embodiments in which the genetically engineered bacteria encode circuitry for the production of one or more STING agonists, the bacteria may further comprise an auxotrophic modification, e.g., a mutation or deletion in DapA, ThyA, or both. In any of these embodiments in which the genetically engineered bacteria encode circuitry for the production of one or more STING agonists, the bacteria may further comprise a phage modification, e.g., a mutation or deletion, in an endogenous prophage as described herein

### STING Agonists and Adenosine Consumption

In one embodiment, the genetically engineered bacteria comprise one or more genes encoding enzymes for the production of STING agonist(s) in combination with one or more genes encoding enzymes for the consumption of adenosine. In one embodiment, the STING agonist produced is c-di-AMP. In one embodiment, the genetically engineered bacteria comprises gene sequences encoding a diadenylate cyclase in combination with gene sequences encoding an adenosine degradation pathway. In one embodiment, the diadenylate cyclase is from Listeria monocytogenes. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the diadenylate cyclase is from Listeria monocytogenes and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In one embodiment, the STING agonist produced is cyclic-di-GAMP. In one embodiment, the genetically engineered bacteria comprises gene sequences encoding a c-di-GAMP synthase in combination with gene sequences encoding a adenosine degradation pathway. In one embodiment, the c-di-GAMP synthases is from Vibrio cholerae. In one embodiment, the c-di-GAMP synthases is human cGAS. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the c-di-GAMP synthase is from Vibrio cholerae and the gene sequences encoding the adenosine degradation pathway comprise xdhA, xdhB, xdhC, add, xapA, deoD, and nupC, e.g., from E. coli. In one specific embodiment, the c-di-GAMP synthase is from human cGAS and the gene sequences encoding the adenosine degradation pathway comprise xdhA, xdhB, xdhC, add, xapA, deoD, and nupC, e.g., from E. coli. In one specific embodiment, the genes encoding the adenosine degradation pathway are chromosomally integrated and under control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the production of STING agonist(s) in combination with comprises genetically engineered bacteria comprising one or more genes encoding enzymes for the consumption of adenosine. In one embodiment, the STING agonist produced is c-di-AMP. In one embodiment, the composition comprises genetically engineered bacteria comprising gene sequences encoding a diadenylate cyclase in combination with genetically engineered bacteria comprising gene sequences encoding an adenosine degradation pathway. In one embodiment, the diadenylate cyclase is from Listeria monocytogenes. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the diadenylate cyclase is from Listeria monocytogenes and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In one embodiment, the STING agonist produced is cyclic-di-GAMP. In one embodiment, the composition comprises genetically engineered bacteria comprising gene sequences encoding a c-di-GAMP synthase in combination with genetically engineered bacteria comprising gene sequences encoding an adenosine degradation pathway. In one embodiment, the c-di-GAMP synthases is from Vibrio cholerae. In one embodiment, the c-di-GAMP synthases is human cGAS.

In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the c-di-GAMP synthase is from Vibrio cholerae and the gene sequences encoding the adenosine degradation pathway comprise xdhA, xdhB, xdhC, add, xapA, deoD, and nupC, e.g., from E. coli. In one specific embodiment, the c-di-GAMP synthase is human cGAS and the gene sequences encoding the adenosine degradation pathway comprise xdhA, xdhB, xdhC, add, xapA, deoD, and nupC, e.g., from E. coli. In one specific embodiment, the genes encoding the adenosine degradation pathway are chromosomally integrated and under control of a low-oxygen promoter, e.g., FNR.

In any of these STING agonist production and adenosine consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In any of these STING agonist production and adenosine consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more STING agonist, e.g., cyclic-di-AMP, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more STING agonist, e.g., cyclic-di-AMP, than unmodified bacteria of the same bacterial subtype under the same conditions.

In yet another embodiment, the genetically engineered bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more adenosine than unmodified bacteria of the same bacterial subtype under the same conditions.

In any of these STING agonist production and adenosine consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ATP than unmodified bacteria of the same bacterial subtype under the same conditions.

In any of these STING agonist production and adenosine consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more urate than unmodified bacteria of the same bacterial subtype under the same conditions.

In any of these STING agonist production and adenosine consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and adenosine consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and adenosine consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and adenosine consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and adenosine consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some STING agonist production and adenosine consumption embodiments, the genetically engineered microorganisms are capable of expressing any one or more of the described circuits for STING agonist production and the degradation of adenosine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the STING agonist production and the degradation of adenosine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these STING agonist production and adenosine consumption embodiments, any one or more of the described STING agonist production circuits and adenosine consumption circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., adenosine, tryptophan, adenosine, arginine) described herein (8) combinations of one or more of such additional circuits.

In any of these embodiments, the one or more bacteria genetically engineered to produce STING agonists and consume adenosine may be administered alone or in combination with one or more immune checkpoint inhibitors described herein, including but not limited to anti-CTLA4, anti-PD1, or anti-PD-L1 antibodies. In any of these embodiments, the one or more bacteria genetically engineered to produce STING agonists and consume adenosine may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered to produce STING agonists and consume adenosine may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to produce STING agonists and consume adenosine may be genetically engineered to produce and secrete or display on their surface one or more immune stimulatory agonists described herein, e.g., agonistic antibodies, including but not limited to anti-OX40, anti-41BB, or anti-GITR antibodies.

### STING Agonists and Arginine Production/Ammonia Consumption

In one embodiment, the genetically engineered bacteria comprise one or more genes encoding enzymes for the production of STING agonist(s) in combination with one or more genes encoding enzymes for the production of arginine and/or consumption of ammonia. In one embodiment, the STING agonist produced is c-di-AMP. In one embodiment, the genetically engineered bacteria comprises gene sequences encoding a diadenylate cyclase in combination with gene sequences encoding an arginine production and/or ammonia consumption pathway. In one embodiment, the diadenylate cyclase is from Listeria monocytogenes. In one embodiment, the gene sequences encoding the arginine production and/or ammonia consumption circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the production of STING agonist(s) in combination with genetically engineered bacteria comprising one or more genes encoding enzymes for the production of arginine. In one embodiment, the STING agonist produced is c-di-AMP. In one embodiment, the composition comprises genetically engineered bacteria comprising gene sequences encoding a diadenylate cyclase in combination with genetically engineered bacteria comprising gene sequences encoding an arginine production pathway. In one embodiment, the diadenylate cyclase is from Listeria monocytogenes. In one embodiment, the gene sequences encoding the arginine production circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR.

In any of these STING agonist production and arginine production and/or ammonia consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In any of these STING agonist production and arginine production and/or ammonia consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more STING agonist, e.g., cyclic-di-AMP, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more STING agonist, e.g., cyclic-di-AMP, than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more arginine than unmodified bacteria of the same bacterial subtype under the same conditions.

In any of these STING agonist production and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these STING agonist production and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more glutamate than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more glutamate than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these STING agonist production and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ammonia than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more ammonia than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ammonia than unmodified bacteria of the same bacterial subtype under the same conditions.

In any of these STING agonist production and arginine production and/or ammonia consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and arginine production and/or ammonia consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and arginine production and/or ammonia consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and arginine production and/or ammonia consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and arginine production and/or ammonia consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some STING agonist production and arginine production/ammonia consumption embodiments, the genetically engineered microorganisms are capable of expressing any one or more of the described circuits for STING agonist production and arginine production/ammonia consumption in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the STING agonist production and arginine production/ammonia consumption are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these STING agonist production and arginine production/ammonia consumption embodiments, any one or more of the described STING agonist production circuits and arginine production/ammonia consumption circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., arginine production/ammonia, tryptophan, arginine production/ammonia, arginine, and kynurenine) described herein (8) combinations of one or more of such additional circuits.

In any of these embodiments, the one or more bacteria genetically engineered to produce STING agonists and produce arginine and/or consume ammonia may be administered alone or in combination with one or more immune checkpoint inhibitors described herein, including but not limited to anti-CTLA4, anti-PD1, or anti-PD-L1 antibodies. In any of these embodiments, the one or more bacteria genetically engineered to produce STING agonists and produce arginine and/or consume ammonia may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered to produce STING agonists and produce arginine and/or consume ammonia may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to produce STING agonists and produce arginine and/or consume ammonia may be genetically engineered to produce and secrete or display on their surface one or more immune stimulatory agonists described herein, e.g., agonistic antibodies, including but not limited to anti-OX40, anti-41BB, or anti-GITR antibodies.

In certain embodiments, one or more genetically engineered bacteria expressing any one or more of the described circuits for the production of one or more STING agonists and one or more kynureninase(s), e.g., from Pseudomonas fluorescens, further comprise circuitry for the secretion or display of a checkpoint inhibitor, e.g., anti-PD-1, anti-PD-L1 or anti-CTLA-4. In one embodiment, one or more genetically engineered bacteria comprise gene sequence(s) encoding DacA, e.g., from Listeria monocytogenes, wherein DacA is operably linked to a promoter inducible under low oxygen conditions, e.g., a FNR promoter. The bacteria comprising gene sequences encoding dacA comprise a mutation or deletion in dapA. The one or more genetically engineered bacteria further comprise gene sequences encoding feedback-resistant ArgA and the bacterium comprising gene sequences encoding feedback-resistant ArgA further comprises a mutation or deletion in ArgR. In certain embodiments, dacA and feedback-resistant ArgA sequences are integrated into the bacterial chromosome. In one specific embodiment, the one or more genetically engineered bacteria may further comprise mutation(s) or deletion(s) in ThyA. In one specific embodiment, the checkpoint inhibitor is PD-1. In one specific embodiment, the checkpoint inhibitor is PD-L1. In one specific embodiment, the checkpoint inhibitor is CTLA-4. The checkpoint inhibitor may be under control of a constitutive or inducible promoter.

In one embodiment, dacA circuitry (e.g., from Listeria monocytogenes, e.g., under control of a low oxygen promoter and chromosomally integrated), arginine production/ammonia consumption circuitry (e.g., comprising ArgAfbr, e.g., under the control of a low oxygen inducible promoter and chromosomally integrated and ΔArgR), circuitry for the secretion or display of a checkpoint inhibitor, e.g., anti-PD-1, anti-PD-L1 or anti-CTLA-4, and auxotrophic mutations (mutations or deletions in dapA, and ThyA) are combined in one bacterium. In an alternate embodiment, a bacterial composition comprises a first bacterium comprising gene sequences encoding dacA (e.g., from Listeria monocytogenes, e.g., under control of a low oxygen promoter and chromosomally integrated), further comprising a mutation or deletion in DapA, and with an optional mutation or deletion in ThyA, and a second bacterium comprising gene sequences encoding arginine production/ammonia consumption circuitry (e.g., comprising ArgAfbr, e.g., under the control of a low oxygen inducible promoter and chromosomally integrated and ΔArgR), and optionally a mutation or deletion in ThyA. The composition further comprises a third bacterium engineered to secrete or display the checkpoint inhibitor. Alternatively the first or second bacterium are engineered to secrete or display the checkpoint inhibitor.

### STING Agonist and Checkpoint Inhibitors

In some embodiments, the one or more genetically engineered bacteria comprise one or more genes encoding enzymes for the production of STING agonist(s) in combination with one or more genes encoding one or more checkpoint inhibitors, e.g., anti-PD-1, anti-PDL-1, and/or anti-CTLA4 antibodies. In some embodiments, the antibodies are secreted from the bacterium. In some embodiments, the antibodies are displayed on the surface of the bacterium. In one embodiment, the STING agonist produced is c-di-AMP. In one embodiment, the genetically engineered bacteria comprises gene sequences encoding a diadenylate cyclase in combination with gene sequences encoding one or more checkpoint inhibitors, e.g., anti-PD-1, anti-PDL-1, and/or anti-CTLA4 antibodies. In some embodiments, the one or more genetically engineered bacteria further comprise a DOM (diffusible outer membrane) mutation, e.g., ΔPAL to improve secretion of the checkpoint inhibitor. In one embodiment, the diadenylate cyclase is from Listeria monocytogenes. In one embodiment, the checkpoint inhibitor is PD-1. In one embodiment, the checkpoint inhibitor is PD-L1. In one embodiment, the checkpoint inhibitor is CTLA-4. In one specific embodiment, the checkpoint inhibition gene circuitry is integrated into the chromosome and is under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the production of STING agonist(s) in combination with genetically engineered bacteria comprising one or more genes encoding one or more checkpoint inhibitors, e.g., anti-PD-1, anti-PDL-1, and/or anti-CTLA4 antibodies. In one embodiment, the STING agonist produced is c-di-AMP. In one embodiment, the composition comprises genetically engineered bacteria comprising gene sequences encoding a diadenylate cyclase in combination with genetically engineered bacteria comprising gene sequences encoding one or more checkpoint inhibitors, e.g., anti-PD-1, anti-PDL-1, and/or anti-CTLA4 antibodies. In some embodiments, the genetically engineered bacteria encoding the checkpoint inhibitor further comprise a DOM (diffusible outer membrane) mutation, e.g., ΔPAL to improve secretion of the checkpoint inhibitor. In one embodiment, the diadenylate cyclase is from Listeria monocytogenes. In one embodiment, the checkpoint inhibitor is PD-1. In one embodiment, the checkpoint inhibitor is PD-L1. In one embodiment, the checkpoint inhibitor is CTLA-4. In one specific embodiment, the checkpoint inhibition gene circuitry is integrated into the chromosome and is under the control of a low-oxygen promoter, e.g., FNR.

In any of these STING agonist and checkpoint inhibitor production embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more STING agonist, e.g., cyclic-di-AMP, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more STING agonist, e.g., cyclic-di-AMP, than unmodified bacteria of the same bacterial subtype under the same conditions.

In any of these STING agonist and checkpoint inhibitor production embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these STING agonist and checkpoint inhibitor production embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and checkpoint inhibitor production embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and checkpoint inhibitor production embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and checkpoint inhibitor production embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and checkpoint inhibitor production embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In any of these STING agonist and checkpoint inhibitor production embodiments, the genetically engineered microorganisms are capable of expressing any one or more of the described circuits for STING agonist production and checkpoint inhibitor production, e.g., anti-PD-1, anti-PD-L1 and/or anti-CTLA-4 production, in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for STING agonist production and checkpoint inhibitor production, e.g., anti-PD-1, anti-PD-L1 and/or anti-CTLA-4 production, are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these STING agonist production and checkpoint inhibitor production, e.g., anti-PD-1, anti-PD-L1 and/or anti-CTLA-4 production embodiments, any one or more of the described STING agonist production circuits and checkpoint inhibitor production circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., arginine production/ammonia, tryptophan, arginine production/ammonia, arginine, and kynurenine) described herein (8) combinations of one or more of such additional circuits.

In any of these embodiments, the one or more bacteria genetically engineered to produce STING agonists and comprise checkpoint inhibitor production circuits may be administered alone or in combination with one or more immune checkpoint inhibitors described herein, including but not limited to anti-CTLA4, anti-PD1, or anti-PD-L1 antibodies. In any of these embodiments, the one or more bacteria genetically engineered to produce STING agonists and comprise checkpoint inhibitor production circuits may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered to produce STING agonists and comprise checkpoint inhibitor production circuits may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to produce STING agonists and comprise checkpoint inhibitor production circuits may be genetically engineered to produce and secrete or display on their surface one or more immune stimulatory agonists described herein, e.g., agonistic antibodies, including but not limited to anti-OX40, anti-41BB, or anti-GITR antibodies.

### STING Agonist and Immune Stimulatory Agonists

In some embodiments, the one or more genetically engineered bacteria comprise one or more genes encoding enzymes for the production of STING agonist(s) in combination with one or more genes encoding one or more immune stimulator agonists, e.g., anti-OX40, anti-41BB, and/or anti-GITR antibodies. In some embodiments, the antibodies are secreted from the bacterium. In some embodiments, the antibodies are displayed on the surface of the bacterium. In one embodiment, the STING agonist produced is c-di-AMP. In one embodiment, the genetically engineered bacteria comprises gene sequences encoding a diadenylate cyclase in combination with gene sequences encoding one or more immune stimulator agonists, e.g., anti-OX40, anti-41BB, and/or anti-GITR antibodies. In some embodiments, the one or more genetically engineered bacteria further comprise a DOM (diffusible outer membrane) mutation, e.g., ΔPAL to improve secretion of the immune stimulator agonist. In one embodiment, the diadenylate cyclase is from Listeria monocytogenes. In one embodiment, the immune stimulator agonist is OX40. In one embodiment, the immune stimulator agonist is 41BB. In one embodiment, the immune stimulator agonist is GITR. In one specific embodiment, the checkpoint inhibition gene circuitry is integrated into the chromosome and is under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the production of STING agonist(s) in combination with genetically engineered bacteria comprising one or more genes encoding one or more immune stimulator agonists, e.g., anti-OX40, anti-41BB, and/or anti-GITR antibodies. In one embodiment, the STING agonist produced is c-di-AMP. In one embodiment, the composition comprises genetically engineered bacteria comprising gene sequences encoding a diadenylate cyclase in combination with genetically engineered bacteria comprising gene sequences encoding one or more immune stimulator agonists, e.g., anti-OX40, anti-41BB, and/or anti-GITR antibodies. In some embodiments, the genetically engineered bacteria encoding the immune stimulator agonist further comprise a DOM (diffusible outer membrane) mutation, e.g., ΔPAL to improve secretion of the immune stimulator agonist. In one embodiment, the diadenylate cyclase is from Listeria monocytogenes. In one embodiment, the immune stimulator agonist is OX40. In one embodiment, the immune stimulator agonist is 41BB. In one embodiment, the immune stimulator agonist is GITR. In one specific embodiment, the checkpoint inhibition gene circuitry is integrated into the chromosome and is under the control of a low-oxygen promoter, e.g., FNR.

In any of these STING agonist and immune stimulator agonist production embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more STING agonist, e.g., cyclic-di-AMP, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more STING agonist, e.g., cyclic-di-AMP, than unmodified bacteria of the same bacterial subtype under the same conditions.

In any of these STING agonist and immune stimulator agonist production embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these STING agonist and immune stimulator agonist production embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and immune stimulator agonist production embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and immune stimulator agonist production embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and immune stimulator agonist production embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist and immune stimulator agonist production embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In any of these STING agonist and immune stimulator agonist production embodiments, the genetically engineered microorganisms are capable of expressing any one or more of the described circuits for STING agonist production and immune stimulator agonist production, e.g., anti-OX40, anti-41BB and/or anti-GITR production, in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for STING agonist production and immune stimulator agonist production, e.g., anti-OX40, anti-41BB and/or anti-GITR production, are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these STING agonist production and immune stimulator agonist production, e.g., anti-OX40, anti-41BB and/or anti-GITR production embodiments, any one or more of the described STING agonist production circuits and immune stimulator agonist production circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., arginine production/ammonia, tryptophan, arginine production/ammonia, arginine, and kynurenine) described herein (8) combinations of one or more of such additional circuits.

In any of these embodiments, the one or more bacteria genetically engineered to produce STING agonists and comprise immune stimulator agonist production circuits may be administered alone or in combination with one or more immune stimulator agonists described herein, including but not limited to anti-CTLA-4, anti-PD-1, or anti-PD-L1 antibodies. In any of these embodiments, the one or more bacteria genetically engineered to produce STING agonists and comprise immune stimulator agonist production circuits may be genetically engineered to produce and secrete or display on their surface one or more immune stimulator agonists described herein, including but not limited to anti-CTLA-4, anti-PD-1, or anti-PD-L1 antibodies.

In any of these embodiments, the one or more bacteria genetically engineered to produce STING agonists and comprise immune stimulator agonist production circuits may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to produce STING agonists and comprise immune stimulator agonist production circuits may be genetically engineered to produce and secrete or display on their surface one or more immune stimulatory agonists described herein, e.g., agonistic antibodies, including but not limited to anti-OX40, anti-41BB, or anti-GITR antibodies.

### 5-FC to 5-FU Combinations

In one embodiment, the genetically engineered bacteria comprise one or more genes encoding enzymes for the conversion of 5-FC to 5-FU in combination with one or more genes encoding enzymes for the consumption of kynurenine. In one embodiment, the genetically engineered bacteria comprises gene sequences encoding a cytosine deaminase in combination with gene sequences encoding a kynureninase. In one embodiment, the cytosine deaminase is from E. coli. In one embodiment, the cytosine deaminase is from yeast. In one embodiment, the kynureninase is from Pseudomonas fluorescens. In one specific embodiment, the cytosine deaminase is from E. coli and the kynureninase is from Pseudomonas fluorescens. In one embodiment, the kynureninase is from Pseudomonas fluorescens. In one specific embodiment, the kynureninase from Pseudomonas fluorescens is chromosomally integrated and under control of a constitutive promoter.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the conversion of 5-FC to 5-FU in combination with genetically engineered bacteria comprising one or more genes encoding enzymes for the consumption of kynurenine. In one embodiment, the composition comprises genetically engineered bacteria comprising gene sequences encoding a cytosine deaminase in combination with genetically engineered bacteria comprising gene sequences encoding a kynureninase. In one embodiment, the cytosine deaminase is from E. coli. In one embodiment, the cytosine deaminase is from yeast. In one embodiment, the kynureninase is from Pseudomonas fluorescens. In one specific embodiment, the cytosine deaminase is from E. coli and the kynureninase is from Pseudomonas fluorescens. In one embodiment, the kynureninase is from Pseudomonas fluorescens. In one specific embodiment, the kynureninase from Pseudomonas fluorescens is chromosomally integrated and under control of a constitutive promoter.

In any of these 5-FC to 5-FU conversion and kynurenine consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In any of these 5-FC to 5-FU conversion and kynurenine consumption embodiments, the genetically engineered bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more kynurenine than unmodified bacteria of the same bacterial subtype under the same conditions.

In yet another embodiment, the genetically engineered bacteria convert 1.0-1.2-fold, 1.2-1.4-fold, 1.4-1.6-fold, 1.6-1.8-fold, 1.8-2-fold, or two-fold more 5-FC into 5-FU, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more 5-FU, than unmodified bacteria of the same bacterial subtype under the same conditions.

In yet another embodiment, the genetically engineered bacteria consume 1.0-1.2-fold, 1.2-1.4-fold, 1.4-1.6-fold, 1.6-1.8-fold, 1.8-2-fold, or two-fold more kynurenine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more kynurenine than unmodified bacteria of the same bacterial subtype under the same conditions.

In any of these 5-FC to 5-FU conversion and kynurenine consumption embodiments, the genetically engineered bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more 5-FC than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more 5-FC than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more 5-FC than unmodified bacteria of the same bacterial subtype under the same conditions.

In any of these 5-FC to 5-FU conversion and kynurenine consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more tryptophan than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more tryptophan than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more tryptophan than unmodified bacteria of the same bacterial subtype under the same conditions.

In any of these 5-FC to 5-FU conversion and kynurenine consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and kynurenine consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and kynurenine consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and kynurenine consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and kynurenine consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In one embodiment, the genetically engineered bacteria comprise one or more genes encoding enzymes for the conversion of 5-FC to 5-FU in combination with one or more genes encoding enzymes for the consumption of adenosine. In one embodiment, the genetically engineered bacteria comprises gene sequences encoding a cytosine deaminase in combination with gene sequences encoding an adenosine degradation pathway. In one embodiment, the cytosine deaminase is from E. coli. In one embodiment, the cytosine deaminase is from yeast. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the cytosine deaminase is from E. coli and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the conversion of 5-FC to 5-FU in combination with one or more genes encoding enzymes for the consumption of adenosine. In one embodiment, the composition comprises genetically engineered bacteria comprising gene sequences encoding a cytosine deaminase in combination with genetically engineered bacteria comprising gene sequences encoding an adenosine degradation pathway. In one embodiment, the cytosine deaminase is from E. coli. In one embodiment, the cytosine deaminase is from yeast. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the cytosine deaminase is from E. coli and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In any of these 5-FC to 5-FU conversion and adenosine consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more STING agonist, e.g., cyclic-di-AMP, than unmodified bacteria of the same bacterial subtype under the same conditions.

In any of these 5-FC to 5-FU conversion and adenosine consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more 5-FU, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more 5-FU, than unmodified bacteria of the same bacterial subtype under the same conditions.

In yet another embodiment, the genetically engineered bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more adenosine than unmodified bacteria of the same bacterial subtype under the same conditions.

In any of these 5-FC to 5-FU conversion and adenosine consumption embodiments, the genetically engineered bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more 5-FC than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more 5-FC than unmodified bacteria of the same bacterial subtype under the same conditions.

In any of these 5-FC to 5-FU conversion and adenosine consumption embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more urate than unmodified bacteria of the same bacterial subtype under the same conditions.

In any of these 5-FC to 5-FU conversion and adenosine consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these STING agonist production and adenosine consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and adenosine consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and adenosine consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and adenosine consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In one embodiment, the genetically engineered bacteria comprise one or more genes encoding enzymes for the production 5-FU in combination with one or more genes encoding enzymes for the production of arginine. In one embodiment, the genetically engineered bacteria comprises gene sequences encoding a cytosine deaminase in combination with gene sequences encoding an arginine production pathway. In one embodiment, the cytosine deaminase is from E. coli. In one embodiment, the cytosine deaminase is from yeast. In one embodiment, the gene sequences encoding the arginine production circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in one embodiment, the composition comprises genetically engineered bacteria comprising one or more genes encoding enzymes for the production 5-FU in combination with genetically engineered bacteria comprising one or more genes encoding enzymes for the production of arginine In one embodiment, the composition comprises genetically engineered bacteria comprising gene sequences encoding a cytosine deaminase in combination with genetically engineered bacteria comprising gene sequences encoding an arginine production pathway. In one embodiment, the cytosine deaminase is from E. coli. In one embodiment, the cytosine deaminase is from yeast. In one embodiment, the gene sequences encoding the arginine production circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR.

In any of these 5-FC to 5-FU conversion and arginine production embodiments, the genetically engineered bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more 5-FU, than unmodified bacteria of the same bacterial subtype under the same conditions.

In any of the 5-FC to 5-FU conversion and arginine production embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more 5-FU, than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more 5-FU than unmodified bacteria of the same bacterial subtype under the same conditions.

In yet another embodiment, the genetically engineered bacteria produce at least about 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more arginine than unmodified bacteria of the same bacterial subtype under the same conditions.

In any of these 5-FC to 5-FU conversion and arginine production embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more 5-FC than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more 5-FC than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more 5FC than unmodified bacteria of the same bacterial subtype under the same conditions.

In any of these 5-FC to 5-FU conversion and arginine production embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more glutamate than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more glutamate than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these 5-FC to 5-FU conversion and arginine production embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and arginine production embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and arginine production embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and arginine production embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these 5-FC to 5-FU conversion and arginine production embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In any of these embodiments combining immune activation and priming with immune augmentation, the gene sequence(s) encoding effectors for targeting immune activation and priming and the gene sequence(s) encoding effectors for immune augmentation may be operably linked to one or more directly or indirectly inducible promoter(s). In some embodiments, the two or more gene sequence(s) are operably linked to a directly or indirectly inducible promoter that is induced under exogeneous environmental conditions, e.g., conditions found in the gut, the tumor microenvironment or other tissue specific conditions. In some embodiments, the two or more gene sequence(s) are operably linked to a directly or indirectly inducible promoter that is induced by metabolites found in the gut, the tumor microenvironment or other specific conditions. In some embodiments, the two or more gene sequence(s) are operably linked to a directly or indirectly inducible promoter that is induced under low-oxygen or anaerobic conditions. In some embodiments, the two or more gene sequence(s) are operably linked to a directly or indirectly inducible promoter that is induced under inflammatory conditions (e.g., RNS, ROS), as described herein. In some embodiments, the two or more gene sequence(s) are operably linked to a directly or indirectly inducible promoter that is induced under immunosuppressive conditions, e.g., as found in the tumor, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, cumate, and salicylate, or others described herein. In some embodiments, the two or more payloads are all linked to a constitutive promoter. Suitable constitutive promoters are described herein. In some embodiments, the two or more gene sequence are operably linked to the same promoter sequences. In some embodiments, the two or more gene sequence are operably linked to two or more different promoter sequences, which can either all be constitutive (same or different constitutive promoters), all inducible (by same or different inducers), or a mix of constitutive and inducible promoters.

In any of the above immune activation and immune augmentation combination embodiments, the gene sequence(s) for producing the one or more immune activation and immune augmentation effectors may be present on a chromosome in the bacterium. In any of the above combination embodiments, the gene sequence(s) for producing the one or more immune activation and immune augmentation effectors may be present on a plasmid in the bacterium. In any of the above combination embodiments, the gene sequence(s) for producing the one or more immune activation and immune augmentation effectors may be present both on a plasmid and the chromosome in the bacterium. In any of the above combination embodiments, the bacterium may be an auxotroph comprising a deletion or mutation in a gene required for cell survival and/or growth, e.g., wherein the gene is selected from thyA, dapD, and dapA. In any of the above combination embodiment, the genetically engineered bacterium may comprise a kill switch.

In some immune initiator and sustainer combinations and/or compositions, the genetically engineered microorganisms are capable of expressing any one or more of the described immune initiator circuits and immune sustainer circuits for the in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some immune initiator and sustainer combinations and/or compositions, the gene sequences(s) encoding circuitry for immune initiators and immune sustainers are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these immune initiator and sustainer combinations and/or compositions, any one or more of the immune initiator circuits and immune sustainer circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits. In any of these embodiments, the bacteria genetically engineered to consume adenosine and kynurenine may be administered alone or in combination with one or more immune checkpoint inhibitors described herein, including but not limited anti-CTLA4, anti-PD1, or anti-PD-L1 antibodies.

In any of these embodiments, in which a microorganism genetically engineered to express one or more immune initiator circuit(s) and immune sustainer circuit(s), the microorganism may first produce higher levels of immune stimulator and at a later time produce immune sustainer. In any of these immune initiator/sustainer embodiments, expression of the immune initiator may be induced by an inducer. In any of these immune initiator/sustainer embodiments, immune sustainer may be induced by an inducer. In any of these immune initiator/sustainer embodiments, both immune initiator and immune sustainer may be induced by one or more inducer(s). A first inducer (inducing immune stimulator expression) and a second inducer (inducing immune sustainer expression) may be the same or different inducers. In any of these immune initiator/sustainer embodiments, the first inducer and second inducer may be administered sequentially or concurrently. Non-limiting examples of inducers include in vivo conditions, e.g., conditions of the gut or the tumor microenvironment (e.g., low oxygen, certain nutrients, etc.), conditions during cell culture or in vitro growth, or chemical inducers (e.g., arabinose, cumate, and salicylate, IPTG or other chemical inducers described herein), which can be employed in vitro or in vivo. In any of these immune initiator/sustainer embodiments, both immune initiator and immune sustainer may be controlled by or directly or indirectly linked to constitutive promoters, including but not limited to those described herein. In any of these immune initiator/sustainer embodiments, the immune initiator may be controlled by or directly or indirectly linked to an inducible promoter and immune sustainer may be controlled by or directly or indirectly linked to a constitutive promoter. In any of these immune initiator/sustainer embodiments, the immune initiator may be controlled by or directly or indirectly linked to an constitutive promoter and the immune sustainer may be controlled by or directly or indirectly linked to an inducible promoter.

In any of these immune initiator/sustainer embodiments, a bacterial strain expressing circuitry for immune initiation may be administered in conjunction with a separate bacterial strain expressing circuitry for immune sustenance. For example, one or more strain(s) of genetically engineered bacteria expressing immune initiatory circuitry and one or more separate strains of genetically engineered bacteria expressing immune sustainer circuitry may be administered sequentially, e.g., immune stimulator may be administered before immune sustainer. In another example, the immune initiator strain may be administered after the immune sustainer strain. In yet another example, the immune initiator strain may be administered concurrently with the immune sustainer strain.

In any of these immune initiator/sustainer embodiments, regardless of the sequence or timing of the administration (concurrent or sequential), engineered strains may express the circuitry for the immune sustainer sequentially or concurrently upon administration, i.e., timing and levels of expression are tuned using one or more mechanisms described herein, including but not limited to promoters and ribosome binding sites.

### Combinations of Metabolic Circuits

### Adenosine and Kynurenine Consumption

In some embodiments, the genetically engineered bacteria comprise circuitry that produces and/or consumes one or more metabolites. Alternatively, the disclosure provides a composition comprising a combination (e.g., two or more) of different genetically engineered bacteria, each bacteria encoding one or more enzymes for the production and/or consumption of one or more metabolic substrates. Such distinct or different bacterial strains can be administered concurrently or sequentially. Non-limiting examples of such substrates include kynurenine, tryptophan, adenosine and arginine.

Each combination of circuits, effectors, or immune modulators described herein can either be provided as combination circuitry in one bacterial strain or alternatively in two or more different or separate bacterial strains each expressing one or more circuits of the combination. For example, one or more genetically engineered bacteria comprising circuitry for the consumption of kynurenine and gene circuitry for the production arginine can be provided in one strain comprising both circuits or in two or more strains, each comprising at least one of the circuits.

In some embodiments, the genetically engineered bacteria comprise circuitry for the degradation of adenosine and the consumption of kynurenine. In one embodiment, the genetically engineered bacteria comprise gene sequences encoding kynureninase in combination with an adenosine degradation pathway. In one embodiment, the kynureninase is from Pseudomonas fluorescens. In one specific embodiment, the kynureninase from Pseudomonas fluorescens is chromosomally integrated and under control of a constitutive promoter. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the kynureninase is from Pseudomonas fluorescens and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR. In any of these embodiments, TrpE may be deleted.

In alternate embodiments, the disclosure provides a composition comprising a combination (e.g., two or more) of different genetically engineered bacteria, each bacteria encoding a different immune modulator. Accordingly, in some embodiments, the composition comprises genetically engineered bacteria comprising circuitry for the degradation of adenosine and genetically engineered bacteria the consumption of kynurenine. In one embodiment, the composition comprises genetically engineered bacteria comprising gene sequences encoding kynureninase in combination with genetically engineered bacteria comprising gene sequences encoding an adenosine degradation pathway. In any of these embodiments, TrpE may be deleted.

In one embodiment, the kynureninase is from Pseudomonas fluorescens. In one specific embodiment, the kynureninase from Pseudomonas fluorescens is chromosomally integrated and under control of a constitutive promoter. In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the kynureninase is from Pseudomonas fluorescens and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR. In any of these embodiments, TrpE may be deleted.

In any of these adenosine and kynurenine consumption embodiments, the bacteria genetically engineered to consume adenosine and kynurenine consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions. In yet another embodiment, the bacteria genetically engineered to consume adenosine and kynurenine consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, bacteria genetically engineered to consume adenosine and kynurenine consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more adenosine than unmodified bacteria of the same bacterial subtype under the same conditions.

In any of these adenosine and kynurenine consumption embodiments, the bacteria genetically engineered to consume adenosine and kynurenine produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the bacteria genetically engineered to consume adenosine and kynurenine produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the bacteria genetically engineered to consume adenosine and kynurenine produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more urate than unmodified bacteria of the same bacterial subtype under the same conditions.

In any of these adenosine and kynurenine consumption embodiments, the bacteria genetically engineered to consume adenosine and kynurenine increase the adenosine degradation rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the bacteria genetically engineered to consume adenosine and kynurenine increase the adenosine degradation rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the bacteria genetically engineered to consume adenosine and kynurenine increase the degradation rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In any of these adenosine and kynurenine consumption embodiments, the bacteria genetically engineered to consume adenosine and kynurenine may have an adenosine degradation rate of about 1.8-10 umol/hr/10\hat{ }9 cells when induced under low oxygen conditions. In one specific embodiment, the bacteria genetically engineered to consume adenosine and kynurenine have an adenosine degradation rate of about 5-9 umol/hr/10\hat{ }9 cells. In one specific embodiment, the bacteria genetically engineered to consume adenosine and kynurenine have an adenosine degradation rate of about 6-8 umol/hr/10\hat{ }9 cells.

In any of these adenosine and kynurenine consumption embodiments, the bacteria genetically engineered to consume adenosine and kynurenine may increase the adenosine degradation by about 50% to 70% relative to unmodified bacteria of the same bacterial subtype under the same conditions, i.e., when induced under low oxygen conditions, after 1 hour. In one embodiment, the bacteria genetically engineered to consume adenosine and kynurenine increase the adenosine degradation by about 55% to 65% relative to unmodified bacteria of the same bacterial subtype under the same conditions, i.e., when induced under low oxygen conditions after 1 hour. In one specific embodiment, the bacteria genetically engineered to consume adenosine and kynurenine increase the adenosine degradation by about 55% to 60% relative to unmodified bacteria of the same bacterial subtype under the same conditions, i.e., when induced under low oxygen conditions, after 1 hour. In yet another embodiment, the bacteria genetically engineered to consume adenosine and kynurenine increase the adenosine degradation by about 1.5-3 fold when induced under low oxygen conditions, after 1 hour. In one specific embodiment, the bacteria genetically engineered to consume adenosine and kynurenine increase the adenosine degradation by about 2-2.5 fold when induced under low oxygen conditions, after 1 hour.

In one adenosine and kynurenine consumption embodiment, the bacteria increase the adenosine degradation by about 85% to 100% relative to unmodified bacteria of the same bacterial subtype under the same conditions, i.e., when induced under low oxygen conditions, after 2 hours. In one embodiment, the bacteria genetically engineered to consume adenosine and kynurenine increase the adenosine degradation by about 95% to 100% relative to unmodified bacteria of the same bacterial subtype under the same conditions, i.e., when induced under low oxygen conditions after 2 hours. In one specific embodiment, the bacteria genetically engineered to consume adenosine and kynurenine increase the adenosine degradation by about 97% to 99% relative to unmodified bacteria of the same bacterial subtype under the same conditions, i.e., when induced under low oxygen conditions, after 2 hours.

In one adenosine and kynurenine consumption embodiment, the bacteria genetically engineered to consume adenosine and kynurenine may increase the adenosine degradation by about 40-50 fold when induced under low oxygen conditions, after 2 hours. In one specific embodiment, the bacteria genetically engineered to consume adenosine and kynurenine increase the adenosine degradation by about 44-48 fold when induced under low oxygen conditions, after 2 hours.

In one adenosine and kynurenine consumption embodiment, the bacteria genetically engineered to consume adenosine and kynurenine increase the adenosine degradation by about 95% to 100% relative to unmodified bacteria of the same bacterial subtype under the same conditions, i.e., when induced under low oxygen conditions, after 3 hours. In one embodiment, the bacteria genetically engineered to consume adenosine and kynurenine increase the adenosine degradation by about 98% to 100% relative to unmodified bacteria of the same bacterial subtype under the same conditions, i.e., when induced under low oxygen conditions after 3 hours. In one specific embodiment, the bacteria genetically engineered to consume adenosine and kynurenine increase the adenosine degradation by about 99% to 99% relative to unmodified bacteria of the same bacterial subtype under the same conditions, i.e., when induced under low oxygen conditions, after 3 hours. In yet another embodiment, the bacteria genetically engineered to consume adenosine and kynurenine increase the adenosine degradation by about 100-1000 fold when induced under low oxygen conditions, after 3 hours. In yet another embodiment, the bacteria genetically engineered to consume adenosine and kynurenine increase the adenosine degradation by about 1000-10000 fold when induced under low oxygen conditions, after 3 hours.

In one adenosine and kynurenine consumption embodiment, the bacteria genetically engineered to consume adenosine and kynurenine increase the adenosine degradation by about 95% to 100% relative to unmodified bacteria of the same bacterial subtype under the same conditions, i.e., when induced under low oxygen conditions, after 4 hours. In one embodiment, the bacteria genetically engineered to consume adenosine and kynurenine increase the adenosine degradation by about 98% to 100% relative to unmodified bacteria of the same bacterial subtype under the same conditions, i.e., when induced under low oxygen conditions after 4 hours. In one embodiment, the bacteria genetically engineered to consume adenosine and kynurenine increase the adenosine degradation by about 99% to 99% relative to unmodified bacteria of the same bacterial subtype under the same conditions, i.e., when induced under low oxygen conditions, after 4 hours. In yet another embodiment, the bacteria genetically engineered to consume adenosine and kynurenine increase the adenosine degradation by about 100-1000 fold when induced under low oxygen conditions, after 4 hours. In yet another embodiment, the bacteria genetically engineered to consume adenosine and kynurenine increase the adenosine degradation by about 1000-10000 fold when induced under low oxygen conditions, after 4 hours.

In any of these adenosine and kynurenine consumption embodiments, the bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more kynurenine than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more kynurenine than unmodified bacteria of the same bacterial subtype under the same conditions. In yet another embodiment, the genetically engineered bacteria consume about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more kynurenine than unmodified bacteria of the same bacterial subtype under the same conditions.

In any of these adenosine and kynurenine consumption embodiments, the bacteria genetically engineered to consume kynurenine and optionally produce tryptophan produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more tryptophan than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more tryptophan than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more tryptophan than unmodified bacteria of the same bacterial subtype under the same conditions.

In any of these adenosine and kynurenine consumption embodiments, the genetically engineered bacteria increase the kynurenine consumption rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another adenosine and kynurenine consumption embodiment, the genetically engineered bacteria increase the kynurenine consumption rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase the kynurenine consumption rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one adenosine and kynurenine consumption embodiment, the genetically engineered bacteria increase the kynurenine consumption by about 80% to 100% relative to unmodified bacteria of the same bacterial subtype under the same conditions, after 4 hours. In one adenosine and kynurenine consumption embodiment, the genetically engineered bacteria increase the kynurenine consumption by about 90% to 100% relative to unmodified bacteria of the same bacterial subtype under the same conditions after 4 hours. In one specific adenosine and kynurenine consumption embodiment, the genetically engineered bacteria increase the kynurenine consumption by about 95% to 100% relative to unmodified bacteria of the same bacterial subtype under the same conditions, after 4 hours. In one specific embodiment, the genetically engineered bacteria increase the kynurenine consumption by about 99% to 100% relative to unmodified bacteria of the same bacterial subtype under the same conditions, after 4 hours. In yet another embodiment, the genetically engineered bacteria increase the kynurenine consumption by about 10-50 fold after 4 hours. In yet another embodiment, the genetically engineered bacteria increase the kynurenine consumption by about 50-100 fold after 4 hours. In yet another embodiment, the genetically engineered bacteria increase the kynurenine consumption by about 100-500 fold after 4 hours. In yet another embodiment, the genetically engineered bacteria increase the kynurenine consumption by about 500-1000 fold after 4 hours. In yet another embodiment, the genetically engineered bacteria increase the kynurenine consumption by about 1000-5000 fold after 4 hours. In yet another embodiment, the genetically engineered bacteria increase the kynurenine consumption by about 5000-10000 fold after 4 hours. In yet another embodiment, the genetically engineered bacteria increase the kynurenine consumption by about 10000-1000 fold after 4 hours.

In any of these adenosine and kynurenine consumption embodiments, the genetically engineered bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these adenosine and kynurenine consumption embodiments, the genetically engineered bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these adenosine and kynurenine consumption embodiments, the genetically engineered bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these adenosine and kynurenine consumption embodiments, the genetically engineered bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these adenosine and kynurenine consumption embodiments, the genetically engineered bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some adenosine and kynurenine consumption embodiments, the genetically engineered microorganisms are capable of expressing any one or more of the described circuits for the degradation of adenosine and kynurenine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the degradation of adenosine and/or kynurenine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these adenosine and kynurenine consumption embodiments, any one or more of the described adenosine degradation circuits and kynurenine consumption circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits.

In any of these embodiments, the one or more bacteria genetically engineered to consume adenosine and kynurenine may be administered alone or in combination with one or more immune checkpoint inhibitors described herein, including but not limited to anti-CTLA4, anti-PD1, or anti-PD-L1 antibodies. In any of these embodiments, the one or more bacteria genetically engineered to consume adenosine and kynurenine may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered to consume adenosine and kynurenine may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered to consume adenosine and kynurenine may be genetically engineered to produce and secrete or display on their surface one or more immune stimulatory agonists described herein, e.g., agonistic antibodies, including but not limited to anti-OX40, anti-41BB, or anti-GITR antibodies.

### Adenosine Consumption and Arginine Production

In some embodiments, the genetically engineered bacteria comprise circuitry for the degradation of adenosine and the production of arginine and/or consumption of ammonia. In one embodiment, the genetically engineered bacteria comprise gene sequences encoding an arginine production and/or ammonia consumption circuit in combination with an adenosine degradation pathway. In one embodiment, the gene sequences encoding the arginine production and/or ammonia consumption circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR.

In one embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the feedback resistant ArgA (ArgAfbr) is from E. coli and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In alternate embodiments, the disclosure provides a composition comprising a combination (e.g., two or more) of different genetically engineered bacteria, each bacteria encoding a different immune modulator. Accordingly, in some embodiments, the composition comprises genetically engineered bacteria comprising circuitry for the degradation of adenosine and genetically engineered bacteria comprising sequences for the production of arginine and/or ammonia consumption. In one embodiment, the composition comprises genetically engineered bacteria comprising gene sequences encoding feedback resistant ArgA (ArgAfbr) and a deletion in the endogenous arginine operon repressor ArgR in combination with genetically engineered bacteria comprising gene sequences encoding an adenosine degradation pathway.

In one composition embodiment, the ArgAfbr is from E. coli. In one specific embodiment, ArgAfbr is integrated into the chromosome and is under the control of a low-oxygen promoter, e.g., FNR. In one composition embodiment, the gene sequences encoding the adenosine degradation pathway enzymes comprise one or more genes selected from xdhA, xdhB, xdhC, add, xapA, deoD, and nupC. In one embodiment, the gene sequences encoding the adenosine degradation pathway comprise xdhA, xdhB, xdhC, add, xapA, deoD, and nupC. In one embodiment, the adenosine pathway enzymes are from E. coli. In one specific embodiment, the diadenylate cyclase is from Listeria monocytogenes and the gene sequences encoding the adenosine degradation pathway comprise xdhA, xdhB, xdhC, add, xapA, deoD, and nupC, e.g., from E. coli. In on specific embodiment, the adenosine pathway enzymes are integrated into the chromosome and are under the control of a low-oxygen promoter, e.g., FNR.

In any of these adenosine consumption and arginine production and/or ammonia consumption embodiments, the bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more adenosine than unmodified bacteria of the same bacterial subtype under the same conditions. In yet another embodiment, the bacteria consume 1.0-1.2-fold, 1.2-1.4-fold, 1.4-1.6-fold, 1.6-1.8-fold, 1.8-2-fold, or two-fold more adenosine than unmodified bacteria of the same bacterial subtype under the same conditions. In yet another embodiment, bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more adenosine than unmodified bacteria of the same bacterial subtype under the same conditions.

In any of these adenosine consumption and arginine production and/or ammonia consumption embodiments, the bacteria produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more urate than unmodified bacteria of the same bacterial subtype under the same conditions. In yet another embodiment, the bacteria produce at least about 1.0-1.2-fold, 1.2-1.4-fold, 1.4-1.6-fold, 1.6-1.8-fold, 1.8-2-fold, or two-fold more urate than unmodified bacteria of the same bacterial subtype under the same conditions. In yet another embodiment, the bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more urate than unmodified bacteria of the same bacterial subtype under the same conditions.

In any of these adenosine consumption and arginine production and/or ammonia consumption embodiments, the bacteria increase the adenosine degradation rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the bacteria increase the adenosine degradation rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the bacteria increase the degradation rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In any of these adenosine consumption and arginine production and/or ammonia consumption embodiments, the bacteria may have an adenosine degradation rate of about 1.8-10 umol/hr/10\hat{ }9 cells when induced under low oxygen conditions. In one specific embodiment, the bacteria have an adenosine degradation rate of about 5-9 umol/hr/10\hat{ }9 cells. In one specific embodiment, the bacteria have an adenosine degradation rate of about 6-8 umol/hr/10\hat{ }9 cells.

In any of these adenosine consumption and arginine production and/or ammonia consumption embodiments, the bacteria may increase the adenosine degradation by about 50% to 70% relative to unmodified bacteria of the same bacterial subtype under the same conditions, i.e., when induced under low oxygen conditions, after 1 hour. In one embodiment, the bacteria increase the adenosine degradation by about 55% to 65% relative to unmodified bacteria of the same bacterial subtype under the same conditions, i.e., when induced under low oxygen conditions after 1 hour. In one specific embodiment, the bacteria increase the adenosine degradation by about 55% to 60% relative to unmodified bacteria of the same bacterial subtype under the same conditions, i.e., when induced under low oxygen conditions, after 1 hour. In yet another embodiment, the bacteria increase the adenosine degradation by about 1.5-3 fold when induced under low oxygen conditions, after 1 hour. In one specific embodiment, the bacteria increase the adenosine degradation by about 2-2.5 fold when induced under low oxygen conditions, after 1 hour.

In one adenosine consumption and arginine production and/or ammonia consumption embodiment, the bacteria increase the adenosine degradation by about 85% to 100% relative to unmodified bacteria of the same bacterial subtype under the same conditions, i.e., when induced under low oxygen conditions, after 2 hours. In one embodiment, the bacteria increase the adenosine degradation by about 95% to 100% relative to unmodified bacteria of the same bacterial subtype under the same conditions, i.e., when induced under low oxygen conditions after 2 hours. In one specific embodiment, the bacteria increase the adenosine degradation by about 97% to 99% relative to unmodified bacteria of the same bacterial subtype under the same conditions, i.e., when induced under low oxygen conditions, after 2 hours.

In one adenosine consumption and arginine production and/or ammonia consumption embodiment, the bacteria may increase the adenosine degradation by about 40-50 fold when induced under low oxygen conditions, after 2 hours. In one specific embodiment, the bacteria increase the adenosine degradation by about 44-48 fold when induced under low oxygen conditions, after 2 hours.

In one adenosine consumption and arginine production and/or ammonia consumption embodiment, the bacteria increase the adenosine degradation by about 95% to 100% relative to unmodified bacteria of the same bacterial subtype under the same conditions, i.e., when induced under low oxygen conditions, after 3 hours. In one embodiment, the bacteria increase the adenosine degradation by about 98% to 100% relative to unmodified bacteria of the same bacterial subtype under the same conditions, i.e., when induced under low oxygen conditions after 3 hours. In one specific embodiment, the bacteria increase the adenosine degradation by about 99% to 99% relative to unmodified bacteria of the same bacterial subtype under the same conditions, i.e., when induced under low oxygen conditions, after 3 hours. In yet another embodiment, the bacteria increase the adenosine degradation by about 100-1000 fold when induced under low oxygen conditions, after 3 hours. In yet another embodiment, the bacteria increase the adenosine degradation by about 1000-10000 fold when induced under low oxygen conditions, after 3 hours.

In one adenosine consumption and arginine production and/or ammonia consumption embodiment, the bacteria increase the adenosine degradation by about 95% to 100% relative to unmodified bacteria of the same bacterial subtype under the same conditions, i.e., when induced under low oxygen conditions, after 4 hours. In one embodiment, the bacteria increase the adenosine degradation by about 98% to 100% relative to unmodified bacteria of the same bacterial subtype under the same conditions, i.e., when induced under low oxygen conditions after 4 hours. In one embodiment, the bacteria increase the adenosine degradation by about 99% to 99% relative to unmodified bacteria of the same bacterial subtype under the same conditions, i.e., when induced under low oxygen conditions, after 4 hours. In yet another embodiment, the bacteria increase the adenosine degradation by about 100-1000 fold when induced under low oxygen conditions, after 4 hours. In yet another embodiment, the bacteria increase the adenosine degradation by about 1000-10000 fold when induced under low oxygen conditions, after 4 hours.

In any adenosine consumption and arginine production and/or ammonia consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions.

In yet another adenosine consumption and arginine production and/or ammonia consumption embodiment, the genetically engineered bacteria produce at least about 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more arginine than unmodified bacteria of the same bacterial subtype under the same conditions.

In any of these adenosine consumption and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more glutamate than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more glutamate than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these adenosine consumption and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ammonia than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more ammonia than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ammonia than unmodified bacteria of the same bacterial subtype under the same conditions.

In any of these adenosine consumption and arginine production and/or ammonia consumption embodiments, the bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some adenosine consumption and arginine production and/or ammonia consumption embodiments, the genetically engineered microorganisms are capable of expressing any one or more of the described circuits for the degradation of adenosine and production of arginine in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for the degradation of adenosine and the production of arginine are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In any of these adenosine consumption and arginine production and/or ammonia consumption embodiments, any one or more of the described adenosine degradation circuits and arginine production circuits are present on one or more plasmids (e.g., high copy or low copy) or are integrated into one or more sites in the microorganismal chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits.

In any of these embodiments, the bacteria genetically engineered to consume adenosine and produce arginine and/or consume ammonia may be administered alone or in combination with one or more immune checkpoint inhibitors described herein, including but not limited anti-CTLA4, anti-PD1, or anti-PD-L1 antibodies. In any of these embodiments, the one or more bacteria genetically engineered consume adenosine and produce arginine and/or consume ammonia may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered consume adenosine and produce arginine and/or consume ammonia may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered consume adenosine and produce arginine and/or consume ammonia may be genetically engineered to produce and secrete or display on their surface one or more immune stimulatory agonists described herein, e.g., agonistic antibodies, including but not limited to anti-OX40, anti-41BB, or anti-GITR antibodies.

### Kynurenine Consumption and Arginine Production

In some embodiments, the genetically engineered bacteria comprise circuitry for the consumption of kynurenine and the production of arginine and or consumption of ammonia. In one embodiment, the genetically engineered bacteria comprise gene sequences encoding an arginine production and/or ammonia consumption circuit in combination with a kynurenine consumption (and optionally tryptophan production) pathway. In one embodiment, the genetically engineered bacteria comprise gene sequences encoding kynureninase in combination with arginine production pathway. In one embodiment, the kynureninase is from Pseudomonas fluorescens. In one specific embodiment, the kynureninase from Pseudomonas fluorescens is chromosomally integrated and under control of a constitutive promoter. In one embodiment, the gene sequences encoding the arginine production and/or ammonia consumption circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR. In any of these embodiments, TrpE may be deleted.

In alternate embodiments, the disclosure provides a composition comprising a combination (e.g., two or more) of different genetically engineered bacteria, each bacteria encoding a different immune modulator. Accordingly, in some embodiments, the composition comprises genetically engineered bacteria comprising circuitry for the consumption of kynurenine and genetically engineered bacteria comprising circuitry for the production of arginine and or consumption of ammonia. In one embodiment, the composition comprises genetically engineered bacteria comprising gene sequences encoding an arginine production circuit in combination with genetically engineered bacteria comprising gene sequences encoding a kynurenine consumption (and optionally tryptophan production) pathway. In one embodiment, the composition comprises genetically engineered bacteria comprising gene sequences encoding kynureninase in combination with genetically engineered bacteria comprising gene sequences encoding an arginine production pathway. In one embodiment, the kynureninase is from Pseudomonas fluorescens. In one specific embodiment, the kynureninase from Pseudomonas fluorescens is chromosomally integrated and under control of a constitutive promoter. In one embodiment, the gene sequences encoding the arginine production and/or ammonia consumption circuit comprise feedback resistant ArgA (ArgAfbr) and a deletion in the endogenous arginine operon repressor ArgR. In one embodiment, the ArgAfbr is from E. coli. In one specific embodiment, ArgAfbr is integrated into the chromosome and is under the control of a low-oxygen promoter, e.g., FNR. In any of these embodiments, TrpE may be deleted.

In any of these kynurenine consumption and arginine production and/or ammonia consumption embodiments, the bacteria consume 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more kynurenine than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more kynurenine than unmodified bacteria of the same bacterial subtype under the same conditions. In yet another embodiment, the genetically engineered bacteria consume about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more kynurenine than unmodified bacteria of the same bacterial subtype under the same conditions.

In any of these kynurenine consumption and arginine production and/or ammonia consumption embodiments, the bacteria genetically engineered to consume kynurenine and optionally produce tryptophan produce at least about 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more tryptophan than unmodified bacteria of the same bacterial subtype under the same conditions. In yet another embodiment, the genetically engineered bacteria produce at least about 1.0-1.2-fold, 1.2-1.4-fold, 1.4-1.6-fold, 1.6-1.8-fold, 1.8-2-fold, or two-fold more tryptophan than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more tryptophan than unmodified bacteria of the same bacterial subtype under the same conditions.

In any of these kynurenine consumption and arginine production and/or ammonia consumption embodiments, the genetically engineered bacteria increase the kynurenine consumption rate by 0% to 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another kynurenine consumption and arginine production embodiment, the genetically engineered bacteria increase the kynurenine consumption rate by 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase the kynurenine consumption rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one kynurenine consumption and arginine production and/or ammonia consumption embodiment, the genetically engineered bacteria increase the kynurenine consumption by about 80% to 100% relative to unmodified bacteria of the same bacterial subtype under the same conditions, after 4 hours. In one kynurenine consumption and arginine production and/or ammonia consumption embodiment, the genetically engineered bacteria increase the kynurenine consumption by about 90% to 100% relative to unmodified bacteria of the same bacterial subtype under the same conditions after 4 hours. In one specific kynurenine consumption and arginine production and/or ammonia consumption embodiment, the genetically engineered bacteria increase the kynurenine consumption by about 95% to 100% relative to unmodified bacteria of the same bacterial subtype under the same conditions, after 4 hours. In one specific embodiment, the genetically engineered bacteria increase the kynurenine consumption by about 99% to 100% relative to unmodified bacteria of the same bacterial subtype under the same conditions, after 4 hours. In yet another embodiment, the genetically engineered bacteria increase the kynurenine consumption by about 10-50 fold after 4 hours. In yet another embodiment, the genetically engineered bacteria increase the kynurenine consumption by about 50-100 fold after 4 hours.

In yet another embodiment, the genetically engineered bacteria increase the kynurenine consumption by about 100-500 fold after 4 hours. In yet another embodiment, the genetically engineered bacteria increase the kynurenine consumption by about 500-1000 fold after 4 hours. In yet another embodiment, the genetically engineered bacteria increase the kynurenine consumption by about 1000-5000 fold after 4 hours. In yet another embodiment, the genetically engineered bacteria increase the kynurenine consumption by about 5000-10000 fold after 4 hours. In yet another embodiment, the genetically engineered bacteria increase the kynurenine consumption by about 10000-1000 fold after 4 hours.

In any kynurenine consumption and arginine production and/or ammonia consumption embodiments, the genetically engineered bacteria produce at least about 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions.

In yet another kynurenine consumption and arginine production and/or ammonia consumption embodiment, the genetically engineered bacteria produce at least about 1.0-1.2-fold, 1.2-1.4-fold, 1.4-1.6-fold, 1.6-1.8-fold, 1.8-2-fold, or two-fold more arginine than unmodified bacteria of the same bacterial subtype under the same conditions. In yet another embodiment, the genetically engineered bacteria produce about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold more arginine than unmodified bacteria of the same bacterial subtype under the same conditions.

In any of these kynurenine consumption and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more glutamate than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more glutamate than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more glutamate than unmodified bacteria of the same bacterial subtype under the same conditions.

In any of these kynurenine consumption and arginine production and/or ammonia consumption embodiments, the genetically engineered bacteria consume 0% to 2%, 2% to 4%, 4% to 6%,6% to 8%, 8% to 10%, 10% to 12%, 12% to 14%, 14% to 16%, 16% to 18%, 18% to 20%, 20% to 25%,25% to 30%, 30% to 35%, 35% to 40%,40% to 45% 45% to 50%, 50% to 55%, 55% to 60%, 60% to 65%, 65% to 70% to 80%, 80% to 90%, or 90% to 100% more ammonia than unmodified bacteria of the same bacterial subtype under the same conditions. In yet another embodiment, the genetically engineered bacteria consume 1.0-1.2-fold, 1.2-1.4-fold, 1.4-1.6-fold, 1.6-1.8-fold, 1.8-2-fold, or two-fold more ammonia than unmodified bacteria of the same bacterial subtype under the same conditions. In yet another embodiment, the genetically engineered bacteria consume about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more ammonia than unmodified bacteria of the same bacterial subtype under the same conditions.

In any of these kynurenine consumption and arginine production and/or ammonia consumption embodiments, the bacteria are capable of reducing cell proliferation by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor growth by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor size by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions. In any of these embodiments, the bacteria are capable of reducing tumor weight by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some kynurenine consumption and arginine production and/or ammonia consumption embodiments, the genetically engineered microorganisms are capable of expressing any one or more of the described circuits for the kynurenine consumption and arginine production in low-oxygen conditions, and/or in the presence of cancer and/or the tumor microenvironment, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose, cumate, and salicylate and others described herein. In some embodiments such an inducer may be administered in vivo to induce effector gene expression. In some embodiments, the gene sequences(s) encoding circuitry for kynurenine consumption and arginine production are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein. In any of these kynurenine consumption and arginine production and/or ammonia consumption embodiments, any one or more of the described adenosine degradation circuits and kynurenine consumption circuits are present on one or more plasmids (e.g., high copy or low copy) or are integrated into one or more sites in the microorganismal chromosome.

Also, in some embodiments, the genetically engineered microorganisms are capable of expressing any one or more of the described circuits and further comprise one or more of the following: (1) one or more auxotrophies, such as any auxotrophies known in the art and provided herein, e.g., thyA and/or dapA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites (e.g., kynurenine, tryptophan, adenosine, arginine) described herein (8) combinations of one or more of such additional circuits.

In any of these embodiments, the bacteria genetically engineered to consume kynurenine and produce arginine and/or consume ammonia may be administered alone or in combination with one or more immune checkpoint inhibitors described herein, including but not limited anti-CTLA4, anti-PD1, or anti-PD-L1 antibodies. In any of these embodiments, the one or more bacteria genetically engineered consume kynurenine and produce arginine and/or consume ammonia may be genetically engineered to produce and secrete or display on their surface one or more immune checkpoint inhibitors described herein, including but not limited to anti-CTLA4, anti-PD1, or anti-PD-L1 antibodies.

In any of these embodiments, the one or more bacteria genetically engineered consume kynurenine and produce arginine and/or consume ammonia may be administered alone or in combination with one or more immune stimulatory agonists described herein, e.g., agonistic antibodies, including but not limited to anti-OX40, anti-41BB, or anti-GITR antibodies. In any of these embodiments, the one or more bacteria genetically engineered consume kynurenine and produce arginine and/or consume ammonia may be genetically engineered to produce and secrete or display on their surface one or more immune stimulatory agonists described herein, e.g., agonistic antibodies, including but not limited to anti-OX40, anti-41BB, or anti-GITR antibodies.

In any of these metabolic converter combination circuit embodiments, (i.e., AD+Kyn, AD+Arg/NH4+, Kyn+Arg) the genetically engineered bacteria comprising gene sequence(s) encoding the metabolic converter combinations further comprise gene sequence(s) encoding one or more further effector molecule(s), i.e., therapeutic molecule(s) or a metabolic converter(s). In any of these embodiments, the circuit encoding the metabolic converter combinations may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding the metabolic converter combinations may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding the metabolic converter combinations encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding the metabolic converter combinations encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome.

In any of these metabolic converter combination embodiments (i.e., AD+Kyn, AD+Arg/NH4+, Kyn+Arg), the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Regulating Expression of Effectors and Immune Modulators

In some embodiments, the bacterial cell comprises a stably maintained plasmid or chromosome carrying the gene(s) encoding payload (s), such that the payload(s) can be expressed in the host cell, and the host cell is capable of survival and/or growth in vitro, e.g., in medium, and/or in vivo, e.g., in the gut or in the tumor microenvironment. In some embodiments, bacterial cell comprises two or more distinct payloads or operons, e.g., two or more payload genes. In some embodiments, bacterial cell comprises three or more distinct transporters or operons, e.g., three or more payload genes. In some embodiments, bacterial cell comprises 4, 5, 6, 7, 8, 9, 10, or more distinct payloads or operons, e.g., 4, 5, 6, 7, 8, 9, 10, or more payload genes.

Herein the terms “payload” “polypeptide of interest” or “polypeptides of interest”, “protein of interest”, “proteins of interest”, “payloads” “effector molecule”, “effector” refers to one or more effector molecules described herein and/or one or more enzyme(s) or polypeptide(s) function as enzymes needed for the production of such effector molecules. Non-limiting examples of payloads include IL-12, IL-2, IL-15, IL-18, IL-7, IL-21, CD40 agonist, CD40 agonist, CD226 agonist, CD137 agonist, ICOS agonist, OXO40 agonist, GM-CSF, CXCL10, CXCL9, antibodies, e.g., scFvs, including but not limited to checkpoint inhibitors (e.g., PD1, PDL1, CTLA4, anti-LAG3, anti-TIM3 and others described herein), kynureninase, tryptophan and/or arginine production enzymes, adenosine degradation enzymes.

As used herein, the term “polypeptide of interest” or “polypeptides of interest”, “protein of interest”, “proteins of interest”, “payload”, “payloads” further includes any or a plurality of any of the tryptophan synthesis enzymes, kynurenine degrading enzymes, adenosine degrading enzymes, arginine producing enzymes, and other metabolic pathway enzymes described herein. As used herein, the term “gene of interest” or “gene sequence of interest” includes any or a plurality of any of the gene(s) an/or gene sequence(s) and or gene cassette(s) encoding one or more immune modulator(s) described herein.

In some embodiments, the genetically engineered bacteria comprise multiple copies of the same payload gene(s). In some embodiments, the gene encoding the payload is present on a plasmid and operably linked to a directly or indirectly inducible promoter. In some embodiments, the gene encoding the payload is present on a plasmid and operably linked to a constitutive promoter. In some embodiments, the gene encoding the payload is present on a plasmid and operably linked to a promoter that is induced under low-oxygen or anaerobic conditions. In some embodiments, the gene encoding the payload is present on plasmid and operably linked to a promoter that is induced by exposure to tetracycline or arabinose, cumate, and salicylate, or another chemical or nutritional inducer described herein.

In some embodiments, the gene encoding the payload is present on a chromosome and operably linked to a directly or indirectly inducible promoter. In some embodiments, the gene encoding the payload is present on a chromosome and operably linked to a constitutive promoter. In some embodiments, the gene encoding the payload is present in the chromosome and operably linked to a promoter that is induced under low-oxygen or anaerobic conditions. In some embodiments, the gene encoding the payload is present on chromosome and operably linked to a promoter that is induced by exposure to tetracycline or arabinose, cumate, and salicylate, or another chemical or nutritional inducer described herein.

In some embodiments, the genetically engineered bacteria comprise two or more payloads, all of which are present on the chromosome. In some embodiments, the genetically engineered bacteria comprise two or more payloads, all of which are present on one or more same or different plasmids. In some embodiments, the genetically engineered bacteria comprise two or more payloads, some of which are present on the chromosome and some of which are present on one or more same or different plasmids.

In any of the embodiments described above, the one or more payload(s) for producing the effector or immune modulator combinations are operably linked to one or more directly or indirectly inducible promoter(s). In some embodiments, the one or more payload(s) are operably linked to a directly or indirectly inducible promoter that is induced under exogeneous environmental conditions, e.g., conditions found in the gut, the tumor microenvironment or other tissue specific conditions. In some embodiments, the one or more payload(s) are operably linked to a directly or indirectly inducible promoter that is induced by metabolites found in the gut, the tumor microenvironment or other specific conditions. In some embodiments, the one or more payload(s) are operably linked to a directly or indirectly inducible promoter that is induced under low-oxygen or anaerobic conditions. In some embodiments, the one or more payload(s) are operably linked to a directly or indirectly inducible promoter that is induced under inflammatory conditions (e.g., RNS, ROS), as described herein. In some embodiments, the one or more payload(s) are operably linked to a directly or indirectly inducible promoter that is induced under immunosuppressive conditions, e.g., as found in the tumor, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, cumate, and salicylate, or others described herein. In some embodiments, the two or more payloads are all linked to a constitutive promoter.

In some embodiments, the promoter is induced under in vivo conditions, e.g., the gut, as described herein. In some embodiments, the promoters is induced under in vitro conditions, e.g., various cell culture and/or cell manufacturing conditions, as described herein. In some embodiments, the promoter is induced under in vivo conditions, e.g., the gut, as described herein, and under in vitro conditions, e.g., various cell culture and/or cell production and/or manufacturing conditions, as described herein.

In some embodiments, the promoter that is operably linked to the gene encoding the payload is directly induced by exogenous environmental conditions (e.g., in vivo and/or in vitro and/or production/manufacturing conditions). In some embodiments, the promoter that is operably linked to the gene encoding the payload is indirectly induced by exogenous environmental conditions (e.g., in vivo and/or in vitro and/or production/manufacturing conditions).

In some embodiments, the promoter is directly or indirectly induced by exogenous environmental conditions specific to the gut of a mammal. In some embodiments, the promoter is directly or indirectly induced by exogenous environmental conditions specific to the hypoxic environment of a tumor and/or the small intestine of a mammal In some embodiments, the promoter is directly or indirectly induced by low-oxygen or anaerobic conditions such as the hypoxic environment of a tumor and/or the environment of the mammalian gut. In some embodiments, the promoter is directly or indirectly induced by molecules or metabolites that are specific to the tumor, a particular tissue or the gut of a mammal In some embodiments, the promoter is directly or indirectly induced by a molecule that is co-administered with the bacterial cell.

### FNR Dependent Regulation

The genetically engineered bacteria of the invention comprise a gene or gene cassette for producing an immune modulator, wherein the gene or gene cassette is operably linked to a directly or indirectly inducible promoter that is controlled by exogenous environmental condition(s). In some embodiments, the inducible promoter is an oxygen level-dependent promoter and an immune modulator is expressed in low-oxygen, microaerobic, or anaerobic conditions. For example, in low oxygen conditions, the oxygen level-dependent promoter is activated by a corresponding oxygen level-sensing transcription factor, thereby driving production of an immune modulator.

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the genetically engineered bacteria comprise a gene or gene cassette for producing a payload under the control of an oxygen level-dependent promoter. In a more specific aspect, the genetically engineered bacteria comprise a gene or gene cassette for producing a payload under the control of an oxygen level-dependent promoter that is activated under low-oxygen or anaerobic environments, such as the hypoxic environment of a tumor and/or the environment of the mammalian gut.

In certain embodiments, the bacterial cell comprises a gene encoding a payload which is operably linked to a fumarate and nitrate reductase regulator (FNR) responsive promoter. In certain embodiments, the bacterial cell comprises a gene encoding a payload expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters of SEQ ID NO: 563-579. Underlined sequences are predicted ribosome binding sites, and bolded sequences are restriction sites used for cloning.

FNR promoter sequences are known in the art, and any suitable FNR promoter sequence(s) may be used in the genetically engineered bacteria of the invention. Any suitable FNR promoter(s) may be combined with any suitable payload.

As used herein the term “payload” refers to one or more effector molecules, e g immune modulator(s), including but not limited to immune initiators and immune sustainers described herein.

Non-limiting FNR promoter sequences are provided in SEQ ID NO: 563-579. In some embodiments, the genetically engineered bacteria of the disclosure comprise a payload, e.g., an effector or an immune modulator, which is operably linked to a low oxygen inducible, e.g., FNR regulated promoter comprising: SEQ ID NO: 563, SEQ ID NO: 564, SEQ ID NO: 565, SEQ ID NO: 566, SEQ ID NO: 567, SEQ ID NO: 568, SEQ ID NO: 569, nirB1 promoter (SEQ ID NO: 570), nirB2 promoter (SEQ ID NO: 571), nirB3 promoter (SEQ ID NO: 572), ydfZ promoter (SEQ ID NO: 573), nirB promoter fused to a strong ribosome binding site (SEQ ID NO: 574), ydfZ promoter fused to a strong ribosome binding site (SEQ ID NO: 575), fnrS, an anaerobically induced small RNA gene (fnrS1 promoter SEQ ID NO: 576 or fnrS2 promoter SEQ ID NO: 577), nirB promoter fused to a crp binding site (SEQ ID NO: 578), and fnrS fused to a crp binding site (SEQ ID NO: 579). In some embodiments, the FNR-responsive promoter is at least about 80%, at least about 85%, at least about 90%, at least about 95%, or at least about 99% homologous to a sequence selected from SEQ ID NOs: 563-579. In another embodiment, the genetically engineered bacteria comprise a gene sequence comprising an FNR-responsive promoter comprising a sequence selected from SEQ ID NOs: 563-579. In yet another embodiment, the FNR-responsive promoter consists of a sequence selected from SEQ ID NOs: 563-579. In some embodiments, the genetically engineered bacteria of the disclosure comprise a gene encoding an effector molecule, e.g., an immune initiator or immune stimulator, which is operably linked to an FNR-responsive promoter which is at least about 80%, at least about 85%, at least about 90%, at least about 95%, or at least about 99% homologous to a sequence selected from SEQ ID NOs: 1281 or SEQ ID NO: 1282. In another embodiment, the genetically engineered bacteria comprise encode an effector molecule operably linked to an FNR-responsive promoter comprising a sequence selected from SEQ ID NOs: 1281 or SEQ ID NO: 1282. In yet another embodiment, the FNR-responsive promoter consists of a sequence selected from SEQ ID NOs: 1281 or SEQ ID NO: 1282.

In some embodiments, multiple distinct FNR nucleic acid sequences are inserted in the genetically engineered bacteria. In alternate embodiments, the genetically engineered bacteria comprise a gene encoding a payload expressed under the control of an alternate oxygen level-dependent promoter, e.g., DNR (Trunk et al., 2010) or ANR (Ray et al., 1997). In these embodiments, expression of the payload gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In another embodiment, the genetically engineered bacteria comprise the gene or gene cassette for producing an immune modulator expressed under the control of anaerobic regulation of arginine deiminase and nitrate reduction transcriptional regulator (ANR). In P. aeruginosa, ANR is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT----ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

In other embodiments, the one or more gene sequence(s) for producing a payload are expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Gorke and Stulke, 2008). In some embodiments, the gene or gene cassette for producing an immune modulator is controlled by an oxygen level-dependent promoter fused to a CRP binding site. In some embodiments, the one or more gene sequence(s) for a payload are controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and transcription of the gene or gene cassette for producing a payload is repressed. In some embodiments, an oxygen level-dependent promoter (e.g., an FNR promoter) fused to a binding site for a transcriptional activator is used to ensure that the gene or gene cassette for producing a payload is not expressed under anaerobic conditions when sufficient amounts of glucose are present, e.g., by adding glucose to growth media in vitro.

In some embodiments, the genetically engineered bacteria comprise an oxygen level-dependent promoter from a different species, strain, or substrain of bacteria. In some embodiments, the genetically engineered bacteria comprise an oxygen level-sensing transcription factor, e.g., FNR, ANR or DNR, from a different species, strain, or substrain of bacteria. In some embodiments, the genetically engineered bacteria comprise an oxygen level-sensing transcription factor and corresponding promoter from a different species, strain, or substrain of bacteria. The heterologous oxygen-level dependent transcriptional regulator and/or promoter increases the transcription of genes operably linked to said promoter, e.g., one or more gene sequence(s) for producing the payload(s) in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the payload, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the payload, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006). In some embodiments, both the oxygen level-sensing transcriptional regulator and corresponding promoter are mutated relative to the wild-type sequences from bacteria of the same subtype in order to increase expression of the payload in low-oxygen conditions.

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the payload are present on different plasmids. In some embodiments, the gene encoding the oxygen level-sensing transcriptional regulator and the gene encoding the payload are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the payload are present on different chromosomes. In some embodiments, the gene encoding the oxygen level-sensing transcriptional regulator and the gene encoding the payload are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the payload. In some embodiments, expression of the transcriptional regulator is controlled by the same promoter that controls expression of the payload. In some embodiments, the transcriptional regulator and the payload are divergently transcribed from a promoter region.

### RNS-Dependent Regulation

In some embodiments, the genetically engineered bacterium that expresses a payload under the control of a promoter that is activated by inflammatory conditions. In one embodiment, the gene for producing the payload is expressed under the control of an inflammatory-dependent promoter that is activated in inflammatory environments, e.g., a reactive nitrogen species or RNS promoter. In some embodiments, the genetically engineered bacteria of the invention comprise a tunable regulatory region that is directly or indirectly controlled by a transcription factor that is capable of sensing at least one reactive nitrogen species. Suitable RNS inducible promoters, e.g., inducible by reactive nitrogen species are described in International Patent Application PCT/US2017/013072, filed Jan. 11, 2017, published as WO2017/123675, the contents of which is herein incorporated by reference in its entirety.

Examples of transcription factors that sense RNS and their corresponding RNS-responsive genes, promoters, and/or regulatory regions include, but are not limited to, those shown in Table 9.

TABLE 9
Examples of RNS-sensing transcription
factors and RNS-responsive genes

| RNS-sensing | Primarily | Examples of responsive |
|---|---|---|
| transcription | capable | genes, promoters, |
| factor: | of sensing: | and/or regulatory regions: |
| NsrR | NO | norB, aniA, nsrR, hmpA, ytfE, |
| | | ygbA, hcp, hcr, nrfA, aox |
| NorR | NO | norVW, nor |
| DNR | NO | norCB, nir, nor, nos |

### ROS-Dependent Regulation

In some embodiments, the genetically engineered bacterium that expresses a payload under the control of a promoter that is activated by conditions of cellular damage. In one embodiment, the gene for producing the payload is expressed under the control of a cellular damaged-dependent promoter that is activated in environments in which there is cellular or tissue damage, e.g., a reactive oxygen species or ROS promoter. In some embodiments, the genetically engineered bacteria of the invention comprise a tunable regulatory region that is directly or indirectly controlled by a transcription factor that is capable of sensing at least one reactive oxygen species. Suitable ROS inducible promoters, e.g., inducible by reactive oxygen species are described in International Patent Application PCT/US2017/013072, filed Jan. 11, 2017, published as WO2017/123675, the contents of which is herein incorporated by reference in its entirety.

Examples of transcription factors that sense ROS and their corresponding ROS-responsive genes, promoters, and/or regulatory regions include, but are not limited to, those shown in Table 10.

TABLE 10
Examples of ROS-sensing
transcription factors and ROS-responsive genes

| ROS-sensing | Primarily | |
|---|---|---|
| transcription | capable | Examples of responsive genes, |
| factor: | of sensing: | promoters, and/or regulatory regions: |
| OxyR | H₂O₂ | ahpC; ahpF; dps; dsbG; fhuF; flu; |
| | | fur; gor; grxA; hemH; katG; oxyS; |
| | | sufA; sufB; sufC; sufD; sufE; sufS; |
| | | trxC; uxuA; yaaA; yaeH; yaiA; ybjM; |
| | | ydcH; ydeN; ygaQ; yljA; ytfK |
| PerR | H₂O₂ | katA; ahpCF; mrgA; zoaA; fur; |
| | | hemAXCDBL; srfA |
| OhrR | Organic | ohrA |
| | peroxides | |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; |
| | | xylC; gabD1; rluC2; cgtS9; azlC; |
| | | narKGHJI; rosR |

### Other Promoters

In some embodiments, the genetically engineered bacteria comprise the gene or gene cassette for producing an immune modulator expressed under the control of an inducible promoter that is responsive to specific molecules or metabolites in the environment, e.g., the tumor microenvironment, a specific tissue, or the mammalian gut. Any molecule or metabolite found in the mammalian gut, in a healthy and/or disease state, or in the tumor microenvironment, may be used to induce payload expression.

In alternate embodiments, the gene or gene cassette for producing an immune modulator is operably linked to a nutritional or chemical inducer which is not present in the environment, e.g., the tumor microenvironment, a specific tissue, or the mammalian gut. In some embodiments, the nutritional or chemical inducer is administered prior, concurrently or sequentially with the genetically engineered bacteria.

### Other Inducible Promoters

In some embodiments, one or more gene sequence(s) encoding polypeptides of interest described herein is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the immune modulator, which is induced by one or more nutritional and/or chemical inducer(s) and/or metabolite(s), such that the immune modulator can be expressed in the host cell, and the host cell is capable of survival and/or growth in vitro, e.g., under culture conditions, and/or in vivo, e.g., in the gut and/or the tumor microenvironment.

In some embodiments, expression of one or more immune modulator(s) and/or other polypeptide(s) of interest is driven directly or indirectly by one or more arabinose, cumate, and salicylate inducible promoter(s) in vivo. In some embodiments, the promoter is directly or indirectly induced by a chemical and/or nutritional inducer and/or metabolite which is co-administered with the genetically engineered bacteria of the invention. In some embodiments, inducers are administered intraperitoneally at a defined time before bacterial injection into the tumor. In some embodiments, inducers are administered intraperitoneally at a defined time after bacterial injection into the tumor. In some embodiments, inducers are administered intraperitoneally concurrently with bacterial injection into the tumor. In some embodiments, inducers are administered intravenously at a defined time before bacterial injection into the tumor. In some embodiments, inducers are administered intravenously at a defined time after bacterial injection into the tumor. In some embodiments, inducers are administered intravenously concurrently with bacterial injection into the tumor. In some embodiments, inducers are administered subcutaneously at a defined time before bacterial injection into the tumor. In some embodiments, inducers are administered subcutaneously at a defined time after bacterial injection into the tumor. In some embodiments, inducers are administered subcutaneously concurrently with bacterial injection into the tumor.

In some embodiments, inducers are administered intratumorally at a defined time before bacterial injection into the tumor. In some embodiments, inducers are administered intratumorally at a defined time after bacterial injection into the tumor. In some embodiments, inducers are administered intratumorally concurrently with bacterial injection into the tumor. In some embodiments, inducers are administered intraperitoneally at a defined time before bacterial injection into the tumor. In some embodiments, inducers are administered intraperitoneally at a defined time after bacterial injection into the tumor. In some embodiments, inducers are administered intraperitoneally concurrently with intravenous bacterial administration. In some embodiments, inducers are administered intravenously at a defined time before bacterial injection into the tumor. In some embodiments, inducers are administered intravenously at a defined time after bacterial injection into the tumor. In some embodiments, inducers are administered intravenously concurrently with intravenous bacterial administration. In some embodiments, inducers are administered subcutaneously at a defined time before bacterial injection into the tumor. In some embodiments, inducers are administered subcutaneously at a defined time after bacterial injection into the tumor. In some embodiments, inducers are administered subcutaneously concurrently with intravenous bacterial administration. In some embodiments, inducers are administered intratumorally at a defined time before bacterial injection into the tumor. In some embodiments, inducers are administered intratumorally at a defined time after bacterial injection into the tumor. In some embodiments, inducers are administered intratumorally concurrently with intravenous bacterial administration.

In some embodiments, expression of one or more immune modulator(s) and/or other polypeptide(s) of interest, is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with one or more immune modulator(s) and/or other polypeptide(s) of interest prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In one embodiment, the gene encoding the effector or the immune modulator is operably linked to a promoter that is induced by salicylate or a derivative thereof. After over 100 years of clinical use, salicylate remains one of the world's most extensively used ‘over-the-counter’ drugs, and it is still recognized as the standard analgesic/antipyretic/anti-inflammatory agent by which newer drugs are assessed (Clissold; Salicylate and related derivatives of salicylic acid; Drugs. 1986; 32 Suppl 4:8-26). In an non-limiting example, the immune modulator is operably linked to a promoter PSal, as part of the salicylate PSal/NahR biosensor circuit (Part:BBa_J61051), originally adapted from Pseudomonas putida. The nahR gene was mined from the 83 kb naphthalene degradation plasmid NAH7 of Pseudomonas putida, encoding a 34 kDa protein which binds to nah and sal promoters to activate transcription in response to the inducer salicylate (Dunn, N. W., and I. C. Gunsalus (1973) Transmissible plasmid encoding early enzymes of naphthalene oxidation in Pseudomonas putida. J. Bacteriol. 114:974-979). In this system NahR is constitutively expressed by a constitutive promoter (Pc), and the expression of the protein of interest, e.g., the immune modulator is positively regulated by NahR in the presence of inducers (e.g., salicylate). Thus, in some embodiments, the genetically engineered bacteria comprise a gene sequence encoding an immune modulator which is operably linked to salicylate inducible promoter (e.g., PSal). In some embodiments, the genetically engineered bacteria further comprise gene sequence(s) encoding NahR, which are operably linked to a promoter. In some embodiments, NahR is under control of a constitutive promoter described herein or known in the art. In some embodiments, NahR is under control of an inducible promoter described herein or known in the art. In some embodiments described herein, the Biobrick BBa_J61051 (containing the gene encoding NahR driven by a constitutive promoter and the PSal promoter was cloned preceding dacA.

In one embodiment, expression of one or more immune modulator protein(s) of interest, e.g., one or more therapeutic polypeptide(s), is driven directly or indirectly by one or more salicylate inducible promoter(s).

In some embodiments, the salicylate inducible promoter is useful for or induced during in vivo expression of the one or more protein(s) of interest. In some embodiments, expression of one or more immune modulator protein(s) of interest is driven directly or indirectly by one or more salicylate inducible promoter(s) in vivo. In some embodiments, the promoter is directly or indirectly induced by a molecule that is co-administered with the genetically engineered bacteria of the invention, e.g., salicylate.

In some embodiments, salicylate is administered intraperitoneally at a defined time before bacterial injection into the tumor. In some embodiments, salicylate is administered intraperitoneally at a defined time after bacterial injection into the tumor. In some embodiments, salicylate is administered intraperitoneally concurrently with bacterial injection into the tumor. In some embodiments, salicylate is administered intravenously at a defined time before bacterial injection into the tumor. In some embodiments, salicylate is administered intravenously at a defined time after bacterial injection into the tumor. In some embodiments, salicylate is administered intravenously concurrently with bacterial injection into the tumor. In some embodiments, salicylate is administered subcutaneously at a defined time before bacterial injection into the tumor. In some embodiments, salicylate is administered subcutaneously at a defined time after bacterial injection into the tumor. In some embodiments, salicylate is administered subcutaneously concurrently with bacterial injection into the tumor.

In some embodiments, salicylate is administered intratumorally at a defined time before bacterial injection into the tumor. In some embodiments, salicylate is administered intratumorally at a defined time after bacterial injection into the tumor. In some embodiments, salicylate is administered intratumorally concurrently with bacterial injection into the tumor. In some embodiments, salicylate is administered intraperitoneally at a defined time before bacterial injection into the tumor. In some embodiments, salicylate is administered intraperitoneally at a defined time after bacterial injection into the tumor. In some embodiments, salicylate is administered intraperitoneally concurrently with intravenous bacterial administration. In some embodiments, salicylate is administered intravenously at a defined time before bacterial injection into the tumor. In some embodiments, salicylate is administered intravenously at a defined time after bacterial injection into the tumor. In some embodiments, salicylate is administered intravenously concurrently with intravenous bacterial administration. In some embodiments, salicylate is administered subcutaneously at a defined time before bacterial injection into the tumor. In some embodiments, salicylate is administered subcutaneously at a defined time after bacterial injection into the tumor. In some embodiments, salicylate is administered subcutaneously concurrently with intravenous bacterial administration. In some embodiments, salicylate is administered intratumorally at a defined time before bacterial injection into the tumor. In some embodiments, salicylate is administered intratumorally at a defined time after bacterial injection into the tumor. In some embodiments, salicylate is administered intratumorally concurrently with intravenous bacterial administration.

In some embodiments, expression of one or more protein(s) of interest, is driven directly or indirectly by one or more salicylate inducible promoter(s) during in vitro growth, preparation, or manufacturing of the strain prior to in vivo administration. In some embodiments, the salicylate inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., salicylate. In some embodiments, the cultures, which are induced by salicylate, are grown aerobically. In some embodiments, the cultures, which are induced by salicylate, are grown anaerobically.

In some embodiments, the salicylate inducible promoter drives the expression of one or more protein(s) of interest from a low-copy plasmid or a high copy plasmid or a biosafety system plasmid described herein. In some embodiments, the salicylate inducible promoter drives the expression of one or more protein(s) of interest from a construct which is integrated into the bacterial chromosome. Exemplary insertion sites are described herein.

In some embodiments, one or more protein(s) of interest are linked to and are driven by the native salicylate inducible promoter In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%,96%, 97%, 98%, or 99% identity with SEQ ID NO: 1273 or SEQ ID NO: 1274.

In one embodiment, the genetically engineered bacteria comprise a gene sequence comprising SEQ ID NO: 1273 or SEQ ID NO: 1274. In another embodiment, the genetically engineered bacteria comprise a gene sequence which consists of SEQ ID NO: 1273 or SEQ ID NO: 1274.

In some embodiments, the salicylate inducible construct further comprises a gene encoding NahR, which in some embodiments is divergently transcribed from a constitutive or inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%,96%, 97%, 98%, or 99% identity with SEQ ID NO: 1278. In another embodiment, the genetically engineered bacteria comprise a gene sequence comprising SEQ ID NO: 1278. In another embodiment, the genetically engineered bacteria comprise a gene sequence which consists of SEQ ID NO: 1278.

In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%,96%, 97%, 98%, or 99% identity with the polypeptide encoded by SEQ ID NO: 1280. In another embodiment, the genetically engineered bacteria comprise a gene sequence encoding a polypeptide comprising SEQ ID NO: 1280. In yet another embodiment, the polypeptide expressed by the genetically engineered bacteria consists of SEQ ID NO: 1280.

In one embodiment, the gene encoding the immune modulator is operably linked to a promoter that is induced by cumate or a derivative thereof. Suitable derivatives are known in the art and are for example described in U.S. Pat. No. 7,745,592. Benefits of cumate induction include that Cumate is non-toxic, water-soluble and inexpensive. The basic mechanism by which the cumate-regulated expression functions in the native P. putida Fl and how it is applied to other bacterial chassis, including but not limited to, E. coli has been previously described (see e.g., Choi et al., Novel, Versatile, and Tightly

Regulated Expression System for Escherichia coli Strains; Appl. Environ. Microbiol. August 2010 vol. 76 no. 15 5058-5066). Essentially, the cumate circuit or switch includes four components: a strong promoter, a repressor-binding DNA sequence or operator, expression of cymR, a repressor, and cumate as the inducer. The addition of the inducer changes causes the formation of a complex between cumate and CymR and results in the removal of the repressor from its DNA binding site, allowing expression of the gene of interest. A construct comprising the cymR gene driven by a constitutive promoter and a cymR responsive promoter was cloned in front of the DacA gene to allow cumate inducible expression of DacA is described elsewhere herein.

In one embodiment, expression of one or more immune modulator protein(s) of interest, e.g., one or more therapeutic polypeptide(s), is driven directly or indirectly by one or more promoter(s) inducible by cumate or a derivative thereof.

In some embodiments, the cumate inducible promoter is useful for or induced during in vivo expression of the one or more protein(s) of interest. In some embodiments, expression of one or more immune modulator protein(s) of interest is driven directly or indirectly by one or more cumate inducible promoter(s) in vivo. In some embodiments, the promoter is directly or indirectly induced by a molecule that is co-administered with the genetically engineered bacteria of the invention, e.g., cumate.

In some embodiments, cumate is administered intraperitoneally at a defined time before bacterial injection into the tumor. In some embodiments, cumate is administered intraperitoneally at a defined time after bacterial injection into the tumor. In some embodiments, cumate is administered intraperitoneally concurrently with bacterial injection into the tumor. In some embodiments, cumate is administered intravenously at a defined time before bacterial injection into the tumor. In some embodiments, cumate is administered intravenously at a defined time after bacterial injection into the tumor. In some embodiments, cumate is administered intravenously concurrently with bacterial injection into the tumor. In some embodiments, cumate is administered subcutaneously at a defined time before bacterial injection into the tumor. In some embodiments, cumate is administered subcutaneously at a defined time after bacterial injection into the tumor. In some embodiments, cumate is administered subcutaneously concurrently with bacterial injection into the tumor.

In some embodiments, cumate is administered intratumorally at a defined time before bacterial injection into the tumor. In some embodiments, cumate is administered intratumorally at a defined time after bacterial injection into the tumor. In some embodiments, cumate is administered intratumorally concurrently with bacterial injection into the tumor. In some embodiments, cumate is administered intraperitoneally at a defined time before bacterial injection into the tumor. In some embodiments, cumate is administered intraperitoneally at a defined time after bacterial injection into the tumor. In some embodiments, cumate is administered intraperitoneally concurrently with intravenous bacterial administration. In some embodiments, cumate is administered intravenously at a defined time before bacterial injection into the tumor. In some embodiments, cumate is administered intravenously at a defined time after bacterial injection into the tumor. In some embodiments, cumate is administered intravenously concurrently with intravenous bacterial administration. In some embodiments, cumate is administered subcutaneously at a defined time before bacterial injection into the tumor. In some embodiments, cumate is administered subcutaneously at a defined time after bacterial injection into the tumor. In some embodiments, cumate is administered subcutaneously concurrently with intravenous bacterial administration. In some embodiments, cumate is administered intratumorally at a defined time before bacterial injection into the tumor. In some embodiments, cumate is administered intratumorally at a defined time after bacterial injection into the tumor. In some embodiments, cumate is administered intratumorally concurrently with intravenous bacterial administration.

In some embodiments, expression of one or more protein(s) of interest, is driven directly or indirectly by one or more cumate inducible promoter(s) during in vitro growth, preparation, or manufacturing of the strain prior to in vivo administration. In some embodiments, the cumate inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., cumate. In some embodiments, the cultures, which are induced by cumate, are grown aerobically. In some embodiments, the cultures, which are induced by cumate, are grown anaerobically.

In some embodiments, the cumate inducible promoter drives the expression of one or more protein(s) of interest from a low-copy plasmid or a high copy plasmid or a biosafety system plasmid described herein. In some embodiments, the cumate inducible promoter drives the expression of one or more protein(s) of interest from a construct which is integrated into the bacterial chromosome. Exemplary insertion sites are described herein.

In some embodiments, one or more protein(s) of interest are operably linked to by the native cumate inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%,96%, 97%, 98%, or 99% identity with SEQ ID NO: 1270 or SEQ ID NO: 1271.

In one embodiment, the genetically engineered bacteria comprise a gene sequence comprising SEQ ID NO: 1270 or SEQ ID NO: 1271. In another embodiment, the genetically engineered bacteria comprise a gene sequence which consists of SEQ ID NO: 1270 or SEQ ID NO: 1271

In some embodiments, the cumate inducible construct further comprises a gene encoding CymR, which in some embodiments is divergently transcribed from a constitutive or inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%,96%, 97%, 98%, or 99% identity with SEQ ID NO: 1268. In another embodiment, the genetically engineered bacteria comprise a gene sequence comprising SEQ ID NO: 1268. In another embodiment, the genetically engineered bacteria comprise a gene sequence which consists of SEQ ID NO: 1268.

In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%,96%, 97%, 98%, or 99% identity with the polypeptide encoded by SEQ ID NO: 1269. In another embodiment, the genetically engineered bacteria comprise a gene sequence encoding a polypeptide comprising SEQ ID NO: 1269. In yet another embodiment, the polypeptide expressed by the genetically engineered bacteria consists of SEQ ID NO: 1269.

Other inducible promoters contemplated in the disclosure are described in are described in International Patent Application PCT/US2017/013072, filed Jan. 11, 2017, published as WO2017/123675, the contents of which is herein incorporated by reference in its entirety. Such promoters include arabinose inducible, rhamnose inducible, and IPTG inducible promoters, tetracycline inducible promoters, temperature inducible promoters, and PSSB promoter. These promoters can be used in combination with each other or with other inducible promoters, such as low oxygen inducible promoters, or constitutive promoters to fine tune expression of different effectors, e.g., in one bacterium or in a composition of more than one strain of bacteria.

### Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut and/or conditions of the tumor microenvironment, as described herein. In some embodiments, the promoters is active under in vitro conditions, e.g., various cell culture and/or cell manufacturing conditions, as described herein. In some embodiments, the constitutive promoter is active under in vivo conditions, e.g., the gut and/or conditions of the tumor microenvironment, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal and/or conditions of the tumor microenvironment. In some embodiments, the constitutive promoter is active in exogenous environmental conditions specific to the small intestine of a mammal. In some embodiments, the constitutive promoter is active in low-oxygen or anaerobic conditions such as the environment of the mammalian gut and/or conditions of the tumor microenvironment. In some embodiments, the constitutive promoter is active in the presence of molecules or metabolites that are specific to the gut of a mammal and/or conditions of the tumor microenvironment. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions. Bacterial constitutive promoters are known in the art and are described in are described in International Patent Application PCT/US2017/013072, filed Jan. 11, 2017, published as WO2017/123675, the contents of which is herein incorporated by reference in its entirety. Examples are included herein in SEQ ID NO: 598-739 and a subset is shown in Table 11.

TABLE 11
Promoters

| | | SEQ |
|---|---|---|
| | | ID |
| Name | Description | NO |
| Plpp | The Plpp promoter is a natural | 740 |
| | promoter taken from the Nissle | |
| | genome. In situ it is used to | |
| | drive production of lpp, which | |
| | is known to be the most | |
| | abundant protein in the cell. | |
| | Also, in some previous RNAseq | |
| | experiments I was able to | |
| | confirm that the lpp mRNA is | |
| | one of the most abundant mRNA | |
| | in Nissle during exponential | |
| | growth. | |
| PapFAB46 | See, e.g., Kosuri, S., Goodman, | 741 |
| | D. B. & Cambray, G. | |
| | Composability of regulatory | |
| | sequences controlling | |
| | transcription and translation | |
| | in Escherichia coli. in 1-20 | |
| | (2013). doi: 10.1073/pnas. | |
| PJ23101 + | UP element helps recruit | 742 |
| UP element | RNA polymerase | |
| | (ggaaaatttttttaaaaaaaaaac | |
| | (SEQ ID NO: 1250)) | |
| PJ23107 + | UP element helps recruit | 743 |
| UP element | RNA polymerase | |
| | (ggaaaatttttttaaaaaaaaaac | |
| | (SEQ ID NO: 1250)) | |
| PSYN23119 | UP element at 5′ end; consensus | 744 |
| | -10 region is TATAAT; the | |
| | consensus -35 is TTGACA; the | |
| | extended -10 region is | |
| | generally TGNTATAAT (TGGTATAAT | |
| | in this sequence | |

In some embodiments, the promoter is Plpp or a derivative thereof. In some embodiments, the promoter comprises a sequence from SEQ ID NO:740. In some embodiments, the constitutive promoter is at least about 80%, at least about 85%, at least about 90%, at least about 95%, or at least about 99% homologous to the sequence of SEQ ID NO: 740. In some embodiments, the promoter is PapFAB46 or a derivative thereof. In some embodiments, the promoter comprises a sequence from SEQ ID NO:741. In some embodiments, the constitutive promoter is at least about 80%, at least about 85%, at least about 90%, at least about 95%, or at least about 99% homologous to the sequence of SEQ ID NO: 741. In some embodiments, the promoter is PJ23101+UP element or a derivative thereof. In some embodiments, the promoter comprises a sequence from SEQ ID NO:742. In some embodiments, the constitutive promoter is at least about 80%, at least about 85%, at least about 90%, at least about 95%, or at least about 99% homologous to the sequence of SEQ ID NO: 742. In some embodiments, the promoter is PJ23107+UP element or a derivative thereof. In some embodiments, the promoter comprises a sequence from SEQ ID NO:743. In some embodiments, the constitutive promoter is at least about 80%, at least about 85%, at least about 90%, at least about 95%, or at least about 99% homologous to the sequence of SEQ ID NO: 743. In some embodiments, the promoter is PSYN23119 or a derivative thereof. In some embodiments, the promoter comprises a sequence from SEQ ID NO:744. In some embodiments, the constitutive promoter is at least about 80%, at least about 85%, at least about 90%, at least about 95%, or at least about 99% homologous to the sequence of SEQ ID NO: 744.

Additional promoters which may be linked to the payload include apFAB124 (tcgacatttatcccttgcggcgaatacttacagccatagcaa (SEQ ID NO: 1443)); apfab338(GGCGCGCC TTGACAATTAATCATCCGGCTCCTAGGATGTGTGGAGGGAC (SEQ ID NO: 1444)), apFAB66 (GGCGCGCC TTGACATCAGGAAAATTTTTCTGTATAATAGATTCATCTCAA (SEQ ID NO: 1445)), and apFAB54 (GGCGCGCC TTGACATAAAGTCTAACCTATAGGATACTTACAGCCATACAAG (SEQ ID NO: 1446)). In some embodiments, the promoter is apFAB124 or a derivative thereof. In some embodiments, the promoter comprises a sequence of apFAB124. In some embodiments, the constitutive promoter is at least about 80%, at least about 85%, at least about 90%, at least about 95%, or at least about 99% homologous to the sequence of apFAB124. In some embodiments, the promoter is apFAB338 or a derivative thereof. In some embodiments, the promoter comprises a sequence of apFAB338. In some embodiments, the constitutive promoter is at least about 80%, at least about 85%, at least about 90%, at least about 95%, or at least about 99% homologous to the sequence of apFAB338. In some embodiments, the promoter is apFAB66 or a derivative thereof. In some embodiments, the promoter comprises a sequence of apFAB66. In some embodiments, the constitutive promoter is at least about 80%, at least about 85%, at least about 90%, at least about 95%, or at least about 99% homologous to the sequence of apFAB66. In some embodiments, the promoter is apFAB54 or a derivative thereof. In some embodiments, the promoter comprises a sequence of apFAB54. In some embodiments, the constitutive promoter is at least about 80%, at least about 85%, at least about 90%, at least about 95%, or at least about 99% homologous to the sequence of apFAB54.

### Ribosome Binding Sites

In some embodiments, ribosome binding sites are added, switched out or replaced. By testing a few ribosome binding sites, expression levels can be fine-tuned to the desired level. In some embodiments, RBS which are suitable for prokaryotic expression and can be used to achieve the desired expression levels are selected. Non-limiting examples of RBS are listed at Registry of standard biological parts and are described in are described in International Patent Application PCT/US2017/013072, filed Jan. 11, 2017, published as WO2017/123675, the contents of which is herein incorporated by reference in its entirety. Suitable examples are shown in SEQ ID NO: 1018-1050 and 869-871, 873-877, 880-887.

### Induction of Payloads During Strain Culture

Induction of payloads during culture is described in International Patent Application PCT/US2017/013072, filed Jan. 11, 2017, published as WO2017/123675, the contents of which is herein incorporated by reference in its entirety.

In some embodiments, it is desirable to pre-induce payload or protein of interest expression and/or payload activity prior to administration. Such payload or protein of interest may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the genetically engineered bacteria of the invention express one or more protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more protein(s) of interest and are directly or indirectly induced, while the strains is grown up for in vivo administration. Without wishing to be bound by theory, pre-induction may boost in vivo activity, e.g., in the gut or a tumor. If the bacterial residence time in a particular gut compartment is relatively short, the bacteria may pass through the small intestine without reaching full in vivo induction capacity. In contrast, if a strain is pre-induced and preloaded, the strains are already fully active, allowing for greater activity more quickly as the bacteria reach the intestine. Ergo, no transit time is “wasted”, in which the strain is not optimally active. As the bacteria continue to move through the intestine, in vivo induction occurs under environmental conditions of the gut (e.g., low oxygen, or in the presence of gut metabolites). Similarly, systemic administration or intratumor injection, as described herein, of other bacterium may allow for greater activity more quickly as the bacteria reach the tumor. Once in the tumor, in vivo induction occurs, e.g., under conditions of the tumor microenvironment.

In one embodiment, expression of one or more payload(s), is induced during cell growth, cell expansion, fermentation, recovery, purification, formulation, and/or manufacture. In one embodiment, expression of several different proteins of interest is induced during cell growth, cell expansion, fermentation, recovery, purification, formulation, and/or manufacture.

In some embodiments, the strains are administered without any pre-induction protocols during strain growth prior to in vivo administration.

### Anaerobic Induction

In some embodiments, cells are induced under anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then switched to anaerobic or low oxygen conditions for approximately 3 to 5 hours. In some embodiments, strains are induced under anaerobic or low oxygen conditions, e.g. to induce FNR promoter activity and drive expression of one or more payload(s) and/or transporters under the control of one or more FNR promoters.

In one embodiment, expression of one or more payload(s), is under the control of one or more FNR promoter(s) and is induced during cell growth, cell expansion, fermentation, recovery, purification, formulation, and/or manufacture under anaerobic or low oxygen conditions. In one embodiment, expression of several different proteins of interest is under the control of one or more FNR promoter(s) and is induced during cell growth, cell expansion, fermentation, recovery, purification, formulation, and/or manufacture under anaerobic or low oxygen conditions.

Without wishing to be bound by theory, strains that comprise one or more payload(s) under the control of an FNR promoter, may allow expression of payload(s) from these promoters in vitro, under anaerobic or low oxygen culture conditions, and in vivo, under the low oxygen conditions found in the gut and/or conditions of the tumor microenvironment.

In some embodiments, promoters linked to the payload of interest may be inducible by arabinose, cumate, and salicylate, IPTG, rhamnose, tetracycline, and/or other chemical and/or nutritional inducers can be induced under anaerobic or low oxygen conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) and/or under the control of one or more FNR promoter(s), and one or more payload gene sequence(s) under the control of a one or more constitutive promoter(s) described herein.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to a permissive temperature, for approximately 3 to 5 hours.

In some embodiments, promoters inducible by arabinose, cumate, and salicylate, IPTG, rhamnose, tetracycline, and/or other chemical and/or nutritional inducers described herein or known in the art can be induced under aerobic conditions in the presence of the chemical and/or nutritional inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut and/or conditions of the tumor microenvironment. In some embodiments, these strains do not further comprise FNR inducible gene sequence(s) for in vivo activation in the gut and/or conditions of the tumor microenvironment.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) are driven by an anaerobic and/or low oxygen promoter, e.g., a FNR promoter. In such instances, cells are for example grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

Without wishing to be bound by theory, strains that comprise one or more payload(s) under the control of an FNR promoter, may allow expression of payload(s) from these promoters in vitro, under microaerobic culture conditions, and in vivo, under the low oxygen conditions found in the gut and/or conditions of the tumor microenvironment.

In some embodiments, promoters inducible by arabinose, cumate, and salicylate, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s), and one or more payload gene sequence(s) under the control of a one or more constitutive promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10\hat{ }8 to 1×10\hat{ }11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e., growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour) to reach an of 0.1 to 10, until the cells are at a density ranging from 1×10\hat{ }8 to 1×10\hat{ }11. Then the chemical inducer, e.g., arabinose, cumate, and salicylate or IPTG, is added to the culture. After 3 to 5 hours, an additional dose of the inducer is added to re-initiate the induction. Spiking can be repeated as needed.

In some embodiments, payload(s) induced during cell growth, cell expansion, fermentation, recovery, purification, formulation, and/or manufacture by using phasing or cycling or pulsing or spiking techniques are under the control of different inducible promoters, for example two different chemical inducers. In other embodiments, the payload is induced under low oxygen conditions or microaerobic conditions and a second payload is induced by a chemical inducer.

### Nucleic Acids

In some embodiments, the disclosure provides novel nucleic acids for producing one or more immune initiators and/or immune sustainers. In some embodiments, the nucleic acid encodes one or more immune initiator and/or immune sustainer polypeptides. Thus, in some embodiments, the nucleic acid comprises gene sequence(s) encoding one or more immune initiator and/or immune sustainer polypeptides.

In some embodiments, the disclosure provides novel nucleic acids for producing one or more STING agonist(s). In some embodiments, the nucleic acid encodes one or more STING agonist producing polypeptides. Thus, in some embodiments, the nucleic acid comprises gene sequence(s) encoding one or more STING agonist producing polypeptides.

In some embodiments, the disclosure provides novel nucleic acids for producing c-diAMP. In some embodiments, the nucleic acid encodes one or more c-diAMP producing polypeptides. Thus, in some embodiments, the nucleic acid comprises gene sequence(s) encoding one or more cyclic diadenylate cyclase polypeptides. In some embodiments, the nucleic acid comprises gene sequence encoding a DacA polypeptide. In certain embodiments, the DacA polypeptide has at least about 80% identity with SEQ ID NO: 1257. In certain embodiments, the DacA polypeptide has at least about 90% identity with SEQ ID NO: 1257. In certain embodiments, the DacA polypeptide has at least about 95% identity with SEQ ID NO: 1257. In some embodiments, the DacA polypeptide has at least about 85%, 86%, 87%, 88%, 89%, 90%, 91%, 92%, 93%, 94%, 95%, 96%, 97%, 98%, or 99% identity with SEQ ID NO: 1257. In some specific embodiments, the DacA polypeptide comprises SEQ ID NO: 1257. In other specific embodiments, the DacA polypeptide consists of SEQ ID NO: 1257. In some embodiments, the nucleic acid comprises a dacA gene sequence. In certain embodiments, the nucleic acid comprising the dacA gene sequence has at least about 80% identity with SEQ ID NO: 1258. In certain embodiments, the nucleic acid comprising the dacA gene sequence has at least about 90% identity with SEQ ID NO: 1258. In certain embodiments, the nucleic acid comprising the dacA gene sequence has at least about 95% identity with SEQ ID NO: 1258. In some embodiments, the nucleic acid comprising the dacA gene sequence has at least about 85%, 86%, 87%, 88%, 89%, 90%, 91%, 92%, 93%, 94%, 95%, 96%, 97%, 98%, or 99% identity with SEQ ID NO: 1258. In some specific embodiments, the nucleic acid comprising the dacA gene sequence comprises SEQ ID NO: 1258. In other specific embodiments the nucleic acid comprising the dacA gene sequence consists of SEQ ID NO: 1258.

In certain embodiments, the nucleic acid comprising the dacA gene sequence is operably linked to a low oxygen inducible promoter. In certain embodiments, the nucleic acid comprising the dacA gene sequence is operably linked to a low oxygen inducible promoter and has at least about 80% identity with SEQ ID NO: 1284. In certain embodiments, the nucleic acid comprising the dacA gene sequence operably linked to a low oxygen inducible promoter has at least about 90% identity with SEQ ID NO: 1284. In certain embodiments, the nucleic acid comprising the dacA gene sequence operably linked to a low oxygen inducible promoter has at least about 95% identity with SEQ ID NO: 1284. In some embodiments, the nucleic acid comprising the dacA gene sequence operably linked to a low oxygen inducible promoter has at least about 85%, 86%, 87%, 88%, 89%, 90%, 91%, 92%, 93%, 94%, 95%, 96%, 97%, 98%, or 99% identity with SEQ ID NO: 1284. In some specific embodiments, the nucleic acid comprising the dacA gene sequence operably linked to a low oxygen inducible promoter comprises SEQ ID NO: 1284. In other specific embodiments the nucleic acid comprising the dacA gene sequence operably linked to a low oxygen inducible promoter consists of SEQ ID NO: 1284.

In some embodiments, the disclosure provides novel nucleic acids for producing c-GAMP. In some embodiments, the nucleic acid encodes one or more c-GAMP producing polypeptides. Thus, in some embodiments, the nucleic acid comprises gene sequence(s) encoding one or more cyclic c-GAMP synthase (cGAS) polypeptides. In some embodiments, the nucleic acid comprises gene sequence encoding a GAMP synthase polypeptide. In some embodiments, the nucleic acid comprises gene sequence encoding a human GAMP synthase polypeptide. In some embodiments, the nucleic acid comprises gene sequence encoding a GAMP synthase polypeptide from Verminephrobacter eiseniae. In some embodiments, the nucleic acid comprises gene sequence encoding a GAMP synthase polypeptide Kingella denitrificans (ATCC 33394). In some embodiments, the nucleic acid comprises gene sequence encoding a GAMP synthase polypeptide Neisseria bacilliformis (ATCC BAA-1200). In certain embodiments, the cGAS polypeptide has at least about 80% identity with a sequence selected from SEQ ID NO: 1254, 1260, 1261, and 1262. In certain embodiments, the cGAS polypeptide has at least about 90% identity with a sequence selected from SEQ ID NO: 1254, 1260, 1261, and 1262. In certain embodiments, the cGAS polypeptide has at least about 95% identity with a sequence selected from SEQ ID NO: 1254, 1260, 1261, and 1262. In some embodiments, the cGAS polypeptide has at least about 85%, 86%, 87%, 88%, 89%, 90%, 91%, 92%, 93%, 94%, 95%, 96%, 97%, 98%, or 99% identity with a sequence selected from SEQ ID NO: 1254, 1260, 1261, and 1262. In some specific embodiments, the cGAS polypeptide comprises a sequence selected from SEQ ID NO: 1254, 1260, 1261, and 1262. In other specific embodiments, the cGAS polypeptide consists of a sequence selected from SEQ ID NO: 1254, 1260, 1261, and 1262. In some embodiments, the nucleic acid comprises a cGAS gene sequence. In certain embodiments, the nucleic acid comprising the cGAS gene sequence has at least about 80% identity with a sequence selected from SEQ ID NO: 1255, 1263, 1264, and 1265. In certain embodiments, the nucleic acid comprising the cGAS gene sequence has at least about 90% identity with a sequence selected from SEQ ID NO: 1255, 1263, 1264, and 1265. In certain embodiments, the nucleic acid comprising the cGAS gene sequence has at least about 95% identity with a sequence selected from SEQ ID NO: 1255, 1263, 1264, and 1265. In some embodiments, the nucleic acid comprising the cGAS gene sequence has at least about 85%, 86%, 87%, 88%, 89%, 90%, 91%, 92%, 93%, 94%, 95%, 96%, 97%, 98%, or 99% identity with a sequence selected from SEQ ID NO: 1255, 1263, 1264, and 1265. In some specific embodiments, the nucleic acid comprising the cGAS gene sequence comprises a sequence selected from SEQ ID NO: 1255, 1263, 1264, and 1265. In other specific embodiments the nucleic acid comprising the cGAS gene sequence consists of a sequence selected from SEQ ID NO: 1255, 1263, 1264, and 1265.

In some embodiments, the disclosure provides novel nucleic acids for depleting kynurenine. In some embodiments, the nucleic acid comprises gene sequence encoding one or more kynurenine depleting enzymes. In one of the nucleic acid embodiments described herein, the nucleic acid sequence encoding the kynurenine catabolism enzyme comprises kynU (Pseudomonas). Accordingly, in one embodiment, the nucleic acid sequence comprising the kynU gene has at least about 80% identity with SEQ ID NO: 68. In one embodiment, the nucleic acid sequence comprising the kynU gene has at least about 90% identity with SEQ ID NO: 68. In another embodiment, the nucleic acid sequence comprising the kynU gene has at least about 95% identity with SEQ ID NO: 68. Accordingly, in one embodiment, the nucleic acid sequence comprising the kynU gene has at least about 85%, 86%, 87%, 88%, 89%, 90%, 91%, 92%, 93%, 94%, 95%, 96%, 97%, 98%, or 99% identity with SEQ ID NO: 68. In another embodiment, the nucleic acid sequence comprising the kynU gene comprises SEQ ID NO: 68. In yet another embodiment, the nucleic acid sequence comprising the kynU gene consists of SEQ ID NO: 68.

In one of the nucleic acid embodiments described herein, the kynurenine degradation enzyme comprises Kynureninase (Pseudomonase fluorescens). In one embodiment, the nucleic acid sequence encodes a polypeptide, which has at least about 80% identity with SEQ ID NO: 65. In one embodiment, the nucleic acid sequence encodes a polypeptide, which has at least about 90% identity with SEQ ID NO: 65. In another embodiment, the nucleic acid sequence encodes a polypeptide, which has at least about 95% identity with SEQ ID NO: 65. Accordingly, in one embodiment, the nucleic acid sequence encodes a polypeptide, which has at least about 85%, 86%, 87%, 88%, 89%, 90%, 91%, 92%, 93%, 94%, 95%, 96%, 97%, 98%, or 99% identity with SEQ ID NO: 65. In another embodiment, the nucleic acid sequence encodes a polypeptide, which comprises a sequence which encodes SEQ ID NO: 65. In yet another embodiment, the nucleic acid sequence encodes a polypeptide, which consists of a sequence which encodes SEQ ID NO: 65.

In some embodiments, the disclosure provides novel nucleic acids for consuming kynurenine. In some embodiments, the nucleic acid encodes one or more kynurenine consuming polypeptides. Thus, in some embodiments, the nucleic acid comprises gene sequence(s) encoding one or more kynurenine consuming polypeptides.

In some embodiments, the disclosure provides novel nucleic acids for consuming kynurenine. In some embodiments, the nucleic acid encodes one or more kynurenine consuming polypeptides. Thus, in some embodiments, the nucleic acid comprises gene sequence(s) encoding one or more kynureninase polypeptides. In some embodiments, the nucleic acid comprises gene sequence encoding a kynureninase polypeptide, e.g., from Pseudomonas fluorescens. In certain embodiments, the kynureninase polypeptide has at least about 80% identity with SEQ ID NO: 65. In certain embodiments, the kynureninase polypeptide has at least about 90% identity with SEQ ID NO: 65. In certain embodiments, the kynureninase polypeptide has at least about 95% identity with SEQ ID NO: 65. In some embodiments, the kynureninase polypeptide has at least about 85%, 86%, 87%, 88%, 89%, 90%, 91%, 92%, 93%, 94%, 95%, 96%, 97%, 98%, or 99% identity with SEQ ID NO: 65. In some specific embodiments, the kynureninase polypeptide comprises SEQ ID NO: 65. In other specific embodiments, the kynureninase polypeptide consists of SEQ ID NO: 65. In some embodiments, the nucleic acid comprises a kyn gene sequence. In certain embodiments, the nucleic acid comprising the kyn gene sequence has at least about 80% identity with SEQ ID NO: 68. In certain embodiments, the nucleic acid comprising the kyn gene sequence has at least about 90% identity with SEQ ID NO: 68. In certain embodiments, the nucleic acid comprising the kyn gene sequence has at least about 95% identity with SEQ ID NO: 68. In some embodiments, the nucleic acid comprising the kyn gene sequence has at least about 85%, 86%, 87%, 88%, 89%, 90%, 91%, 92%, 93%, 94%, 95%, 96%, 97%, 98%, or 99% identity with SEQ ID NO: 68. In some specific embodiments, the nucleic acid comprising the kyn gene sequence comprises SEQ ID NO: 68. In other specific embodiments the nucleic acid comprising the kyn gene sequence consists of SEQ ID NO: 68. In some embodiments, the nucleic acid comprises a kyn gene sequence. In certain embodiments, the nucleic acid comprising the kyn gene sequence has at least about 80% identity with SEQ ID NO: 1283. In certain embodiments, the nucleic acid comprising the kyn gene sequence has at least about 90% identity with SEQ ID NO: 1283. In certain embodiments, the nucleic acid comprising the kyn gene sequence has at least about 95% identity with SEQ ID NO: 1283. In some embodiments, the nucleic acid comprising the kyn gene sequence has at least about 85%, 86%, 87%, 88%, 89%, 90%, 91%, 92%, 93%, 94%, 95%, 96%, 97%, 98%, or 99% identity with SEQ ID NO: 1283. In some specific embodiments, the nucleic acid comprising the kyn gene sequence comprises SEQ ID NO: 1283. In other specific embodiments the nucleic acid comprising the kyn gene sequence consists of SEQ ID NO: 1283.

In certain embodiments, the nucleic acid comprising the kyn gene sequence is operably linked to a constitutive promoter as described herein, e.g., PSYN23119. In certain embodiments, the nucleic acid comprising the kyn gene sequence is operably linked to a low oxygen inducible promoter and has at least about 80% identity with SEQ ID NO: 890. In certain embodiments, the nucleic acid comprising the kyn gene sequence operably linked to a low oxygen inducible promoter has at least about 90% identity with SEQ ID NO: 890. In certain embodiments, the nucleic acid comprising the kyn gene sequence operably linked to a low oxygen inducible promoter has at least about 95% identity with SEQ ID NO: 890. In some embodiments, the nucleic acid comprising the kyn gene sequence operably linked to a low oxygen inducible promoter has at least about 85%, 86%, 87%, 88%, 89%, 90%, 91%, 92%, 93%, 94%, 95%, 96%, 97%, 98%, or 99% identity with SEQ ID NO: 890. In some specific embodiments, the nucleic acid comprising the kyn gene sequence operably linked to a low oxygen inducible promoter comprises SEQ ID NO: 890. In other specific embodiments the nucleic acid comprising the kyn gene sequence operably linked to a low oxygen inducible promoter consists of SEQ ID NO: 890.

Additional suitable nucleic acid sequences are described in International Patent Application PCT/US2017/013072, filed Jan. 11, 2017, published as WO2017/123675, the contents of which is herein incorporated by reference in its entirety.

In any of the nucleic acid embodiments described above, the one or more nucleic acid sequence(s) for producing the effectors, e.g., immune modulators, e.g., immune initiators and or immune sustainers, are operably linked to one or more directly or indirectly inducible promoter(s). In some embodiments, the one or more nucleic acid sequence(s) are operably linked to a directly or indirectly inducible promoter that is induced under exogeneous environmental conditions, e.g., conditions found in the gut, the tumor microenvironment or other tissue specific conditions. In some embodiments, the one or more nucleic acid sequence(s) are operably linked to a directly or indirectly inducible promoter that is induced by metabolites found in the gut, the tumor microenvironment or other specific conditions. In some embodiments, the one or more nucleic acid sequence(s) are operably linked to a directly or indirectly inducible promoter that is induced under low-oxygen or anaerobic conditions. In some embodiments, the one or more nucleic acid sequence(s) are operably linked to a directly or indirectly inducible promoter that is induced under inflammatory conditions (e.g., RNS, ROS), as described herein. In some embodiments, the one or more nucleic acid sequence(s) are operably linked to a directly or indirectly inducible promoter that is induced under immunosuppressive conditions, e.g., as found in the tumor, as described herein. In some embodiments, the one or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, cumate, and salicylate, or others described herein. In some embodiments, the one or more payloads are linked to a constitutive promoter as described herein and in International Patent Application PCT/US2017/013072, filed Jan. 11, 2017, published as WO2017/123675, the contents of which is herein incorporated by reference in its entirety. In some embodiments, the one or more gene sequence are operably linked to the same promoter sequences. In some embodiments, the two or more gene sequence are operably linked to two or more different promoter sequences, which can either all be constitutive (same or different constitutive promoters), all inducible (by same or different inducers), or a mix of constitutive and inducible promoters.

In one embodiment, the one or more nucleic acid sequence(s) encoding one or more immune modulators is located on a plasmid in the bacterial cell. In another embodiment, the one or more nucleic acid sequence(s) encoding one or more immune modulators is located in the chromosome of the bacterial cell. In any of these nucleic acid embodiments, such the one or more nucleic acid sequence(s) encoding one or more immune modulators can be combined with any other nucleic acids encoding other immune modulators described herein.

In any of these embodiments, the nucleic acid sequence(s) encoding effector molecules, e.g., immune modulators, e.g., immune sustainers or immune modulators, described supra and elsewhere herein, may be combined with nucleic acid sequence(s) encoding one or more further effector molecule(s), i.e., therapeutic molecule(s) or a metabolic converter(s) (on the same or on a separate nucleic acid molecule). The nucleic acid sequences encoding the immune initiators or immune sustainers may be under the control of a constitutive or inducible promoter, e.g., low oxygen inducible promoter or any other constitutive or inducible promoter described herein.

In any of these embodiments, the nucleic acid sequence(s) encoding one or more of the effector molecules described supra may be combined with nucleic acid sequence(s) encoding one or more STING agonist producing enzymes, as described herein, (on the same or on a separate nucleic acid molecule). In some embodiments, the nucleic acid sequence(s) which are combined with the nucleic acid sequence(s) encoding the effector molecules encode DacA. DacA may be under the control of a constitutive or inducible promoter, e.g., low oxygen inducible promoter such as FNR or any other constitutive or inducible promoter described herein. In some embodiments, the nucleic acid sequence(s) which are combined with the gene sequence(s) encoding the effector molecules encode cGAS. cGAS may be under the control of a constitutive or inducible promoter, e.g., low oxygen inducible promoter such as FNR or any other constitutive or inducible promoter described herein.

In any of these combination embodiments, the nucleic acid sequence(s) comprise an nucleic acid sequence(s) with an auxotrophic modification, e.g., a mutation or deletion in DapA, ThyA, or both. In any of these embodiments, the nucleic acid sequence(s) comprise a nucleic acid sequence comprising a phage modification, e.g., a mutation or deletion, in an endogenous prophage as described herein.

### Secretion

In any of the embodiments described herein, in which the genetically engineered microorganism produces a protein, polypeptide, peptide, or other immune modulatory, DNA, RNA, small molecule or other molecule intended to be secreted from the microorganism, the engineered microorganism may comprise a secretion mechanism and corresponding gene sequence(s) encoding the secretion system.

In some embodiments, the genetically engineered bacteria further comprise a native secretion mechanism or non-native secretion mechanism that is capable of secreting the immune modulator from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

Suitable secretion systems for secretion of heterologous polypeptides, e.g., effector molecules, from gram negative and gram positive bacteria are described in International Patent Application PCT/US2017/013072, filed Jan. 11, 2017, published as WO2017/123675, the contents of which is herein incorporated by reference in its entirety. Such secretion systems include Double membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps, and type VII secretion system (T7SS). Alternatively, hemolysin-based secretion systems, Type V autotransporter secretion systems, traditional or modified type III or a type III-like secretion systems (T3SS), a flagellar type III secretion pathway may be used. In some embodiments, non-native single membrane-spanning secretion systems, e.g. Tat or Tat-like systems or Sec or Sec like systems may be used. Any of the secretion systems described herein and in PCT/US2017/013072 may according to the disclosure be employed to secrete the polypeptides of interest.

One way to secrete properly folded proteins in gram-negative bacteria—particularly those requiring disulphide bonds—is to target the reducing-environment periplasm in conjunction with a destabilizing outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane (DOM). Destabilizing the bacterial outer membrane to induce leakiness can be accomplished by deleting or mutagenizing genes responsible for tethering the outer membrane to the rigid peptidoglycan skeleton, including for example, 1pp, ompC, ompA, ompF, tolA, tolB, and pal. Lpp is the most abundant polypeptide in the bacterial cell existing at ˜500,000 copies per cell and functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpl can induce leaky phenotypes by promoting an excessive build-up of periplasmic protein. Mutation of the proteases can also preserve the effector polypeptide by preventing targeted degradation by these proteases.

Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated 1pp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from 1pp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from 1pp, ompA, ompF, tolA, tolB, pal, degS, degP, and nlpl, under the control of an inducible promoter. For example, expression of 1pp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, wherein peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

In some embodiments in which the one or more proteins of interest or therapeutic proteins are secreted or exported from the microorganism, the engineered microorganism comprises gene sequence(s) that includes a secretion tag. In some embodiments, the one or more proteins of interest or therapeutic proteins include a “secretion tag” of either RNA or peptide origin to direct the one or more proteins of interest or therapeutic proteins to specific secretion systems. The secretion tag can be from the sec or the that system.

In some embodiments, the genetically engineered bacterial comprise a native or non-native secretion system described herein for the secretion of an immune modulator, e.g., a cytokine, antibody (e.g., scFv), metabolic enzyme (e.g., kynureninase), and others described herein.

In some embodiments, the secretion tag is selected from PhoA, OmpF, cvaC, TorA, fdnG, dmsA, PelB, HlyA secretion signal, and HlyA secretion signal. In some embodiments, the secretion tag is the PhoA secretion signal. In some embodiments, the secretion tag comprises a sequence selected from SEQ ID NO: 745 or SEQ ID NO: 746. In some embodiments, the secretion tag is the OmpF secretion signal. In some embodiments, the secretion tag is the OmpF secretion signal. In some embodiments, the secretion tag comprises SEQ ID NO: 747. In some embodiments, the secretion tag is the cvaC secretion signal. In some embodiments, the secretion tag comprises SEQ ID NO: 748. In some embodiments, the secretion tag is the torA secretion signal. In some embodiments, the secretion tag comprises SEQ ID NO: 749. In some embodiments, the secretion tag is the fdnG secretion signal. In some embodiments, the secretion tag comprises SEQ ID NO: 750. In some embodiments, the secretion tag is the dmsA secretion signal. In some embodiments, the secretion tag comprises SEQ ID NO: 751. In some embodiments, the secretion tag is the PelB secretion signal. In some embodiments, the secretion tag comprises SEQ ID NO: 752. In some embodiments, the secretion tag is the HlyA secretion signal. In some embodiments, the secretion tag comprises a sequence selected from SEQ ID NO: 753 and SEQ ID NO: 754.

In some embodiments, the genetically engineered bacteria encode a polypeptide comprising a secretion tag selected from Adhesin (ECOLIN_19880), DsbA (ECOLIN_21525), GltI (ECOLIN_03430), GspD (ECOLIN_16495), HdeB (ECOLIN_19410), MalE (ECOLIN_22540), OppA (ECOLIN_07295), PelB, PhoA (ECOLIN_02255), PpiA (ECOLIN_18620), TolB, tort, OmpA, PelB, DsbA mglB, and lamB secretion tags. Exemplary sequences of secretion tags are shown in SEQ ID NO: 1222, SEQ ID NO: 1223, SEQ ID NO: 1224, SEQ ID NO: 1225, SEQ ID NO: 1226, SEQ ID NO: 1227, SEQ ID NO: 1228, SEQ ID NO: 1229, SEQ ID NO: 1230, SEQ ID NO: 1141, SEQ ID NO: 1142, SEQ ID NO: 1143, SEQ ID NO: 1144, SEQ ID NO: 1145, SEQ ID NO: 1253, SEQ ID NO: 1157, SEQ ID NO: 1158, SEQ ID NO: 1159, SEQ ID NO: 1160, SEQ ID NO: 1161, SEQ ID NO: 1162, SEQ ID NO: 1163, SEQ ID NO: 1164, SEQ ID NO: 1165, SEQ ID NO: 1166, and SEQ ID NO: 1167.

In some embodiments, a secretion tag polypeptide sequence may be selected from SEQ ID NO: 1218, SEQ ID NO: 1219, SEQ ID NO: 1181, SEQ ID NO: 1220, SEQ ID NO: 1221, SEQ ID NO: 1180, SEQ ID NO: 1184, SEQ ID NO: 1186, SEQ ID NO: 1190, SEQ ID NO: 1182, SEQ ID NO: 1135, SEQ ID NO: 1183, SEQ ID NO: 1188, SEQ ID NO: 1187, SEQ ID NO: 747, SEQ ID NO: 1185, and SEQ ID NO: 1189.

Any secretion tag or secretion system can be combined with any immune modulator described herein intended for secretion. In some embodiments, the secretion system is used in combination with one or more genomic mutations, which leads to the leaky or diffusible outer membrane phenotype (DOM), including but not limited to, 1pp, nlP, tolA, PAL. In some embodiments, the therapeutic proteins secreted by the genetically engineered bacteria are modified to increase resistance to proteases, e.g. intestinal proteases.

In some embodiments, the therapeutic polypeptides of interest, e.g., the immune modulators, e.g., immune initiators and/or immune sustainers described herein, are secreted via a diffusible outer membrane (DOM) system. In some embodiments, the therapeutic polypeptide of interest is fused to a N-terminal Sec-dependent secretion signal. Non-limiting examples of such N-terminal Sec-dependent secretion signals include PhoA, OmpF, OmpA, and cvaC. In alternate embodiments, the therapeutic polypeptide of interest is fused to a Tat-dependent secretion signal. Exemplary Tat-dependent tags include TorA, FdnG, and DmsA.

In certain embodiments, the genetically engineered bacteria comprise deletions or mutations in one or more of the outer membrane and/or periplasmic proteins. Non-limiting examples of such proteins, one or more of which may be deleted or mutated, include 1pp, pal, tolA, and/or nlpI. In some embodiments, 1pp is deleted or mutated. In some embodiments, pal is deleted or mutated. In some embodiments, tolA is deleted or mutated. In other embodiments, nlpI is deleted or mutated. In yet other embodiments, certain periplasmic proteases are deleted or mutated, e.g., to increase stability of the polypeptide in the periplasm. Non-limiting examples of such proteases include degP and ompT. In some embodiments, degP is deleted or mutated. In some embodiments, ompT is deleted or mutated. In some embodiments, degP and ompT are deleted or mutated.

### Surface Display

In some embodiments, the genetically engineered bacteria and/or microorganisms encode one or more gene(s) and/or gene cassette(s) encoding an immune modulator which is anchored or displayed on the surface of the bacteria and/or microorganisms. Examples of the immune modulators which are displayed or anchored to the bacteria and/or microorganism, are any of the immune modulators described herein, and include but are not limited to antibodies, e.g., scFv fragments, and tumor-specific antigens or neoantigens. In a non-limiting example, the antibodies or scFv fragments which are anchored or displayed on the bacterial cell surface are directed against checkpoint inhibitors described herein, including, but not limited to, CLTLA4, PD-1, PD-L1.

Suitable systems for surface display of heterologous polypeptides, e.g., effector molecules, on the surface of gram negative and gram positive bacteria are described in International Patent Application PCT/US2017/013072, filed Jan. 11, 2017, published as WO2017/123675, the contents of which is herein incorporated by reference in its entirety

In some embodiments, the genetically engineered bacteria comprise a gene sequence encoding a therapeutic polypeptide comprising an invasion display tag. In one embodiment, the genetically engineered bacteria comprise a gene sequence encoding a polypeptide comprising SEQ ID NO: 990.

In some embodiments, the genetically engineered bacteria comprise a gene sequence encoding a therapeutic polypeptide comprising an LppOmpA display tag. In one embodiment, the genetically engineered bacteria comprise a gene sequence encoding a polypeptide comprising SEQ ID NO: 991.

In some embodiments, the genetically engineered bacteria comprise a gene sequence encoding a therapeutic polypeptide comprising an intimin N display tag. In one embodiment, the genetically engineered bacteria comprise a gene sequence encoding a polypeptide comprising SEQ ID NO: 992. In some embodiments, the genetically engineered bacteria comprise a display anchor which is at least about 80%, at least about 85%, at least about 90%, at least about 95%, or at least about 99% homologous to a sequence selected from SEQ ID NO: 990, SEQ ID NO: 991, and SEQ ID NO: 992. In another embodiment, the genetically engineered bacteria comprise a gene sequence encoding display anchor comprising a sequence selected from SEQ ID NO: 990, SEQ ID NO: 991, and SEQ ID NO: 992. In yet another embodiment, the display anchor expressed by the genetically engineered bacteria consists of a sequence selected from SEQ ID NO: 990, SEQ ID NO: 991, and SEQ ID NO: 992.

In some embodiments, one or more ScFvs are displayed on the bacterial cell surface, alone or in combination with other therapeutic polypeptides of interest.

In some embodiments, a cell surface display strategy or circuit is combined with a secretion strategy or circuit in one bacterium. In some embodiments, the same polypeptide is both displayed and secreted. In some embodiments, a first polypeptide is displayed and a second is secreted. In some embodiments, a display strategy or circuit strategy is combined with a circuit for the intracellular production of an enzyme and consequentially intracellular catabolism of its substrate. In some embodiments, a display strategy or display circuit is combined with a circuit for the intracellular production of a gut barrier enhancer molecule and/or an anti-inflammatory effector molecule.

In some embodiments, the expression of the surface displayed polypeptide or fusion protein is driven by an inducible promoter. In some embodiments, the inducible promoter is an oxygen level-dependent promoter (e.g., FNR-inducible promoter). In some embodiments, the inducible promoter is induced by gut-specific and/or tumor-specific or promoters induced by inflammation or an inflammatory response (RNS, ROS promoters), or promoters induced by a metabolite that may or may not be naturally present (e.g., can be exogenously added) in the gut, e.g., arabinose, cumate, and salicylate. In alternate embodiments, expression of the surface displayed polypeptides or polypeptide fusion proteins is driven by a constitutive promoter.

In some embodiments, the expression of the surface displayed polypeptide or fusion protein is plasmid based. In some embodiments, the gene sequence(s) encoding the antibodies or scFv fragments for surface display is chromosomally inserted.

### Essential Genes, Auxotrophs, Kill Switches, and Host-Plasmid Dependency

As used herein, the term “essential gene” refers to a gene that is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is a DNA synthesis gene, for example, thyA. In another embodiment, the essential gene is a bacterial cell wall synthesis gene, for example, dapA. In yet another embodiment, the essential gene is an amino acid gene, for example, serA or metA. Any gene required for cell survival and/or growth may be targeted, including but not limited to, cysE, ginA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria. Exemplary bacterial genes which may be disrupted or deleted to produce an auxotrophic strain as described in International Patent Application PCT/US2017/013072, filed Jan. 11, 2017, published as WO2017/123675, the contents of which is herein incorporated by reference in its entirety. These include, but are not limited to, genes required for oligonucleotide synthesis, amino acid synthesis, and cell wall synthesis. Table 12 lists exemplary bacterial genes which may be disrupted or deleted to produce an auxotrophic strain. These include, but are not limited to, genes required for oligonucleotide synthesis, amino acid synthesis, and cell wall synthesis.

TABLE 12
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Auxotrophic mutations are useful in some instances in which biocontainment strategies may be required to prevent unintended proliferation of the genetically engineered bacterium in a natural ecosystem. Any auxotrophic mutation in an essential gene described above or known in the art can be useful for this purpose, e.g. DNA synthesis genes, amino acid synthesis genes, or genes for the synthesis of cell wall. Accordingly, in some embodiments, the genetically engineered bacteria comprise modifications, e.g., mutation(s) or deletion(s) in one or more auxotrophic genes, e.g., to prevent growth and proliferation of the bacterium in the natural environment. In some embodiments, the modification may be located in a non-coding region. In some embodiments, the modifications result in attenuation of transcription or translation. In some embodiments, the modifications, e.g., mutations or deletions, result in reduced or no transcription or reduced or no translation of the essential gene. In some embodiments, the modifications, e.g., mutations or deletions, result in transcription and/or translation of a non-functional version of the essential gene. In some embodiments, the modifications, e.g., mutations or deletions result in truncated transcription or translation of the essential gene, resulting in a truncated polypeptide. In some embodiments, the modification, e.g., mutation is located within the coding region of the gene.

While unable to grow in the natural ecosystem, certain auxotrophic mutations may allow growth and proliferation in the mammalian host administered the bacteria, e.g., in the tumor environment. For example, an essential pathway that is rendered non-functional by the auxotrophic mutation may be complemented by production of the metabolite by the host within the tumor microenvironment. As a result, the bacterium administered to the host can take up the metabolite from the environment and can proliferate and colonize the tumor. Thus, in some embodiments, the auxotrophic gene is an essential gene for the production of a metabolite, which is also produced by the mammalian host in vivo, e.g., in a tumor setting. In some embodiments, metabolite production by the host tumor may allow uptake of the metabolite by the bacterium and permit survival and/or proliferation of the bacterium within the tumor. In some embodiments, bacteria comprising such auxotrophic mutations are capable of proliferating and colonizing the tumor to the same extent as a bacterium of the same subtype which does not carry the auxotrophic mutation.

In some embodiments, the bacteria are capable of colonizing and proliferating in the tumor microenvironment. In some embodiments, the tumor colonizing bacteria comprise one or more auxotrophic mutations. In some embodiments, the tumor colonizing bacteria do not comprise one or more auxotrophic modifications or mutations. In a non-limiting example, greater numbers of bacteria are detected after 24 hours and 72 hours than were originally injected into the subject. In some embodiments,

CFUs detected 24 hours post injection are at least about 1 to 2 logs greater than administered. In some embodiments, CFUs detected 24 hours post injection are at least about 2 to 3 logs greater than administered. In some embodiments, CFUs detected 24 hours post injection are at least about 3 to 4 logs greater than administered. In some embodiments, CFUs detected 24 hours post injection are at least about 4 to 5 logs greater than administered. In some embodiments, CFUs detected 24 hours post injection are at least about 5 to 6 logs greater than administered. In some embodiments, CFUs detected 72 hours post injection are at least about 1 to 2 logs greater than administered. In some embodiments, CFUs detected 72 hours post injection are at least about 2 to 3 logs greater than administered. In some embodiments, CFUs detected 72 hours post injection are at least about 3 to 4 logs greater than administered. In some embodiments, CFUs detected 72 hours post injection are at least about 4 to 5 logs greater than administered. In some embodiments, CFUs detected 72 hours post injection are at least about 5 to 6 logs greater than administered. In some embodiments, CFUs can be measured at later time points, such as after at least one week, after at least 2 or more weeks, after at least one month, after at least two or more months post injection.

Non-limiting examples of such auxotrophic genes, which allow proliferation and colonization of the tumor, are thyA and uraA, as shown herein. Accordingly, in some embodiments, the genetically engineered bacteria of the disclosure may comprise an auxotrophic modification, e.g., mutation or deletion, in the thyA gene. In some embodiments, the genetically engineered bacteria of the disclosure may comprise an auxotrophic modification, e.g., mutation or deletion, in the uraA gene. In some embodiments, the genetically engineered bacteria of the disclosure may comprise auxotrophic modification, e.g., mutation or deletion, in the thyA gene and the uraA gene.

Alternatively, the auxotrophic gene is an essential gene for the production of a metabolite which cannot be produced by the host within the tumor, i.e, the auxotrophic mutation is not complemented by production of the metabolite by the host within the tumor microenvironment. As a result, the this mutation may affect the ability of the bacteria to grow and colonize the tumor and bacterial counts decrease over time. This type of auxotrophic mutation can be useful for the modulation of in vivo activity of the immune modulator or duration of activity of the immune modulator, e.g., within a tumor. An example of this method of fine-tuning levels and timing of immune modulator release is described herein using a auxotrophic modification, e.g, mutation, in dapA. Diaminopimelic acid (Dap) is a characteristic component of certain bacterial cell walls, e.g., of gram negative bacteria. Without diaminopimelic acid, bacteria are unable to form proteoglycan, and as such are unable to grow. DapA is not produced by mammalian cells, and therefore no alternate source of DapA is provided in the tumor. As such, a dapA auxotrophy may present a particularly useful strategy to modulate and fine tune timing and extent of bacterial presence in the tumor and/or levels and timing of immune modulator expression and production. Accordingly, in some embodiments, the genetically engineered bacteria of the disclosure comprise an mutation in an essential gene for the production of a metabolite which cannot be produced by the host within the tumor. In some embodiments, the auxotrophic mutation is in a gene which is essential for the production and maintenance of the bacterial cell wall known in the art or described herein, or a mutation in a gene that is essential to another structure that is unique to bacteria and not present in mammalian cells. In some embodiments, bacteria comprising such auxotrophic mutations are capable of proliferating and colonizing the tumor to a substantially lesser extent than a bacterium of the same subtype which does not carry the auxotrophic mutation. Control of bacterial growth (and by extent effector levels) may be further combined with other regulatory strategies, including but not limited to, metabolite or chemically inducible promoters described herein.

In a non-limiting example, lower numbers of bacteria are detected after 24 hours and 72 hours than were originally injected into the subject. In some embodiments, CFUs detected 24 hours post injection are at least about 1 to 2 logs lower than administered. In some embodiments, CFUs detected 24 hours post injection are at least about 2 to 3 logs lower than administered. In some embodiments, CFUs detected 24 hours post injection are at least about 3 to 4 logs lower than administered. In some embodiments, CFUs detected 24 hours post injection are at least about 4 to 5 logs lower than administered. In some embodiments, CFUs detected 24 hours post injection are at least about 5 to 6 logs lower than administered. In some embodiments, CFUs detected 72 hours post injection are at least about 1 to 2 logs lower than administered. In some embodiments, CFUs detected 72 hours post injection are at least about 2 to 3 logs lower than administered. In some embodiments, CFUs detected 72 hours post injection are at least about 3 to 4 logs lower than administered. In some embodiments, CFUs detected 72 hours post injection are at least about 4 to 5 logs lower than administered. In some embodiments, CFUs detected 72 hours post injection are at least about 5 to 6 logs lower than administered. In some embodiments, CFUs can be measured at later time points, such as after at least one week, after at least 2 or more weeks, after at least one month, after at least two or more months post injection.

In some embodiments, the genetically engineered bacteria of the disclosure comprise a auxotrophic modification, e.g., mutation, in dapA. A non-limiting example described herein is a genetically engineered bacterium comprising gene sequences encoding dacA for c-di-AMP production. Production of the STING agonist can be temporally regulated or restricted through the introduction of a dapA auxotrophy. In some embodiments, the dapA auxotrophy provides a means for tunable STING agonist production.

Auxotrophic modifications may also be used to screen for mutant bacteria that produce the effector molecule for various applications. In one example, the auxotrophy is useful to monitor purity or “sterility” of batches in small and large scale production of a bacterial strain. In this case, the auxotrophy presents a means to distinguish the engineered bacterium from a potential contaminant. In a non-limiting example, during the manufacturing process of the live biotherapeutic (i.e., large scale), an auxotrophy can be a useful tool to demonstrate purity or “sterility” of the drug substance. This method to determine purity of the culture is particularly useful in the absence of an antibiotic resistance gene, which is often used for this purpose in experimental strains, but which may be removed during the development of the live therapeutic drug product.

trpE is another auxotrophic mutation described herein. Bacteria carrying this mutation cannot produce tryptophan. Genetically engineered bacteria described herein with a trpE mutation further comprise kynureninase. Kynureninase allows the bacterium to convert kynurenine into the tryptophan precursor anthranilate and therefore the bacterium can grow in the absence of tryptophan if kynurenine is present.

In some embodiments, the genetically engineered bacteria comprise auxotrophic mutation(s) in one essential gene. In some embodiments, the genetically engineered bacteria comprise auxotrophic mutation(s) in two essential genes (double auxotrophy). In some embodiments, the genetically engineered bacteria comprise auxotrophic mutation(s) in three or more essential gene(s).

In some embodiments, the genetically engineered bacteria comprise auxotrophic mutation(s) in dapA and thyA. In some embodiments, the genetically engineered bacteria comprise auxotrophic mutation(s) in dapA and uraA. In some embodiments, the genetically engineered bacteria comprise auxotrophic mutation(s) in thyA and uraA. In some embodiments, the genetically engineered bacteria comprise auxotrophic mutation(s) in dapA, thyA and uraA.

In some embodiments, the genetically engineered bacteria comprise auxotrophic mutation(s) in trpE. In some embodiments, the genetically engineered bacteria comprise auxotrophic mutation(s) in trpE and thyA. In some embodiments, the genetically engineered bacteria comprise auxotrophic mutation(s) in trpE and dapA. In some embodiments, the genetically engineered bacteria comprise auxotrophic mutation(s) in trpE and uraA. In some embodiments, the genetically engineered bacteria comprise auxotrophic mutation(s) in trpE, dapA and thyA. In some embodiments, the genetically engineered bacteria comprise auxotrophic mutation(s) in trpE, dapA and uraA. In some embodiments, the genetically engineered bacteria comprise auxotrophic mutation(s) in trpE, thyA and uraA. In some embodiments, the genetically engineered bacteria comprise auxotrophic mutation(s) in trpE, dapA, thyA and uraA.

In another non-limiting example, a conditional auxotroph can be generated. The chromosomal copy of dapA or thyA is knocked out. Another copy of thyA or dapA is introduced, e.g., under control of a low oxygen promoter. Under anaerobic conditions, dapA or thyA—as the case may be-are expressed, and the strain can grow in the absence of dap or thymidine. Under aerobic conditions, dapA or thyA expression is shut off, and the strain cannot grow in the absence of dap or thymidine. Such a strategy can also be employed to allow survival of bacteria under anaerobic conditions, e.g., the gut or conditions of the tumor microenvironment, but prevent survival under aerobic conditions.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference). SLiDE bacterial cells are described in International Patent Application PCT/US2017/013072, filed Jan. 11, 2017, published as WO2017/123675, the contents of which is herein incorporated by reference in its entirety.

In some embodiments, the genetically engineered bacteria of the invention also comprise a kill switch. Suitable kill switches are described in International Patent Application PCT/US2016/39427, filed Jun. 24, 2016, published as WO2016/210373, the contents of which are herein incorporated by reference in their entirety. The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

In some embodiments, the genetically engineered bacteria of the invention also comprise a plasmid that has been modified to create a host-plasmid mutual dependency. In certain embodiments, the mutually dependent host-plasmid platform is as described in Wright et al., 2015. These and other systems and platforms are described in International Patent Application PCT/US2017/013072, filed Jan. 11, 2017, published as WO2017/123675, the contents of which is herein incorporated by reference in its entirety.

### Genetic Regulatory Circuits

In some embodiments, the genetically engineered bacteria comprise multi-layered genetic regulatory circuits for expressing the constructs described herein. Suitable multi-layered genetic regulatory circuits are described in International Patent Application PCT/US2016/39434, filed on Jun. 24, 2016, published as WO2016/210378, the contents of which is herein incorporated by reference in its entirety. The genetic regulatory circuits are useful to screen for mutant bacteria that produce an immune modulator or rescue an auxotroph. In certain embodiments, the invention provides methods for selecting genetically engineered bacteria that produce one or more genes of interest.

### Pharmaceutical Compositions and Formulations

Pharmaceutical compositions comprising the genetically engineered microorganisms of the invention may be used to treat, manage, ameliorate, and/or prevent cancer. Pharmaceutical compositions of the invention comprising one or more genetically engineered bacteria, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., one or more genes encoding one or more effectors, e.g., immune modulators. In alternate embodiments, the pharmaceutical composition comprises two or more species, strains, and/or subtypes of bacteria that are each engineered to comprise the genetic modifications described herein, e.g., one or more genes encoding one or more effectors, e.g., immune modulators.

In some embodiments, the genetically engineered bacteria are administered systemically or intratumorally as spores. As a non-limiting example, the genetically engineered bacteria are Clostridial strains, and administration results in a selective colonization of hypoxic/necrotic areas within the tumor. In some embodiments, the spores germinate exclusively in the hypoxic/necrotic regions present in solid tumors and nowhere else in the body.

The pharmaceutical compositions of the invention may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection. Alternatively, the genetically engineered microorganisms may be administered intratumorally and/or peritumorally. In other embodiments, the genetically engineered microorganisms may be administered intra-arterially, intramuscularly, or intraperitoneally. In some embodiments, the genetically engineered bacteria colonize about 20%, 30%, 40%, 50%, 60%, 70%, 80%, 90% or more of the tumor. In some embodiments, the genetically engineered bacteria are co-administered with a PEGylated form of rHuPH20 (PEGPH20) or other agent in order to destroy the tumor septae in order to enhance penetration of the tumor capsule, collagen, and/or stroma. In some embodiments, the genetically engineered bacteria are capable of producing an immune modulator as well as one or more enzymes that degrade fibrous tissue.

The genetically engineered microorganisms of the disclosure may be administered via intratumoral injection, resulting in bacteria or virus that is directly deposited within the target tumor. Intratumoral injection of the engineered bacteria or virus may elicit a potent localized inflammatory response as well as an adaptive immune response against tumor cells. Bacteria or virus are suspended in solution before being withdrawn into a 1-ml syringe. In some embodiments, the tumor is injected with an 18-gauge multipronged needle (Quadra-Fuse, Rex Medical). The injection site is aseptically prepared. If available, ultrasound or CT may be used to identify a necrotic region of the tumor for injection. If a necrotic region is not identified, the injection can be directed to the center of the tumor. The needle is inserted once into a predefined region, and dispensed with even pressure. The injection needle is removed slowly, and the injection site is sterilized.

Direct intratumoral injection of the genetically engineered bacteria or virus of the invention into solid tumors may be advantageous as compared to intravenous administration. Using an intravenous injection method, only a small proportion of the bacteria may reach the target tumor. For example, following E. coli Nissle injection into the tail vein of 4T1 tumor-bearing mice, most bacteria (>99%) are quickly cleared from the animals and only a small percentage of the administered bacteria colonize the tumor (Stritzker et al., 2007). In particular, in large animals and human patients, which have relatively large blood volumes and relatively small tumors compared to mice, intratumoral injection may be especially beneficial. Injection directly into the tumor allows the delivery of a higher concentration of therapeutic agent and avoids the toxicity, which can result from systemic administration. In addition, intratumoral injection of bacteria induces robust and localized immune responses within the tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. Prior to the intratumor administration procedures, sedation in combination with a local anesthetic and standard cardiac, pressure, and oxygen monitoring, or full anesthesia of the patient is performed.

For some tumors, percutaneous injection can be employed, which is the least invasive administration method. Ultrasound, computed tomography (CT) or fluoroscopy can be used as guidance to introduce and position the needle. Percutaneous intratumoral injection is for example described for hepatocellular carcinoma in Lencioni et al., 2010. Intratumoral injection of cutaneous, subcutaneous, and nodal tumors is for example described in WO/2014/036412 (Amgen) for late stage melanoma.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the tumor is larger than the radial reach of the needle. The needle can be pulled back without exiting, and redirected as often as necessary until the full dose is injected and dispersed. To maintain sterility, a separate needle is used for each injection. Needle size and length varies depending on the tumor type and size.

In some embodiments, the tumor is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the tumor and can be positioned at the deepest margin of the tumor. The prongs are deployed to the margins of the tumor. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

Therapeutic endoscopic ultrasonography (EUS) is employed to overcome the anatomical constraints inherent in gaining access to certain other tumors (Shirley et al., 2013). EUS-guided fine needle injection (EUS-FNI) has been successfully used for antitumor therapies for the treatment of head and neck, esophageal, pancreatic, hepatic, and adrenal masses (Verna et al, 2008). EUS-FNI has been extensively used for pancreatic cancer injections. Fine-needle injection requires the use of the curvilinear echoendoscope. The esophagus is carefully intubated and the echoendoscope is passed into the stomach and duodenum where the pancreatic examination occurs, and the target tumor is identified. The largest plane is measured to estimate the tumor volume and to calculate the injection volume. The appropriate volume is drawn into a syringe. A primed 22-gauge fine needle aspiration (FNA) needle is passed into the working channel of the echoendoscope. Under ultrasound guidance, the needle is passed into the tumor. Depending on the size of the tumor, administration can be performed by dividing the tumor into sections and then injecting the corresponding fractions of the volume into each section. Use of an installed endoscopic ultrasound processor with Doppler technology assures there are no arterial or venous structures that may interfere with the needle passage into the tumor (Shirley et al., 2013). In some embodiments, ‘multiple injectable needle’ (MIN) for EUS-FNI can be used to improvement the injection distribution to the tumor in comparison with straight-type needles (Ohara et al., 2013).

Intratumoral administration for lung cancer, such as non-small cell lung cancer, can be achieved through endobronchial intratumor delivery methods, as described in Celikoglu et al., 2008. Bronchoscopy (trans-nasal or oral) is conducted to visualize the lesion to be treated. The tumor volume can be estimated visually from visible length-width height measurements over the bronchial surface. The needle device is then introduced through the working channel of the bronchoscope. The needle catheter, which consists of a metallic needle attached to a plastic catheter, is placed within a sheath to prevent damage by the needle to the working channel during advancement. The needle size and length varies and is determined according to tumor type and size of the tumor. Needles made from plastic are less rigid than metal needles and are ideal, since they can be passed around sharper bends in the working channel. The needle is inserted into the lesion and the genetically engineered bacteria of the invention are in injected. Needles are inserted repeatedly at several insertion points until the tumor mass is completely perfused. After each injection, the needle is withdrawn entirely from the tumor and is then embedded at another location. At the end of the bronchoscopic injection session, removal of any necrotic debris caused by the treatment may be removed using mechanical dissection, or other ablation techniques accompanied by irrigation and aspiration.

In some embodiments, the genetically engineered bacteria or virus capable of delivering an immune modulator to a target tumor are administrated directly into the tumor using methods, including but not limited to, percutaneous injection, EUS-FNI, or endobronchial intratumor delivery methods. In some cases, other techniques, such as laparoscopic or open surgical techniques are used to access the target tumor, however, these techniques are much more invasive and bring with them much greater morbidity and longer hospital stays.

In some embodiments, bacteria, e.g., E. coli Nissle, or spores, e.g., Clostridium novyi NT, are dissolved in sterile phosphate buffered saline (PBS) for systemic or intratumor injection.

The dose to be injected is derived from the type and size of the tumor. The dose of a drug or the genetically engineered bacteria or virus of the invention is typically lower, e.g., orders of magnitude lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

For example, as is for example described in WO/2014/036412, for tumors larger than 5 cm in their largest dimension, up to 4 ml can be injected. For tumors between 2.5 and 5 cm in their largest dimension, up to 2 ml can be injected. For tumors between 2.5 and 5 cm in their largest dimension, up to 2 ml can be injected. For tumors between 1.5 and 2.5 cm in their largest dimension, up to 1 ml can be injected. For tumors between 0.5 and 1.5 cm in their largest dimension, up to 0.5 ml can be injected. For tumors equal or small than 0.5 in their largest dimension, up to 0.1 ml can be injected. Alternatively, ultrasound scan can be used to determine the injection volume that can be taken up by the tumor without leakage into surrounding tissue.

In some embodiments, the treatment regimen will include one or more intratumoral administrations. In some embodiments, a treatment regimen will include an initial dose, which followed by at least one subsequent dose. One or more doses can be administered sequentially in two or more cycles.

For example, a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

In one embodiment, Clostridium spores are delivered systemically. In another embodiment, Clostridium spores are delivered via intratumor injection. In one embodiment, E. coli Nissle are delivered via intratumor injection. In other embodiments, E. coli Nissle, which is known to hone to tumors, is administered via intravenous injection or orally, as described in a mouse model in for example in Danino et al. 2015, or Stritzker et al., 2007, the contents of which is herein incorporated by reference in its entirety. E. coli Nissle mutations to reduce toxicity include but are not limited to msbB mutants resulting in non-myristoylated LPS and reduced endotoxin activity, as described in Stritzker et al., 2010 (Stritzker et al, Bioengineered Bugs 1:2, 139-145; Myristylation negative msbB-mutants of probiotic E. coli Nissle 1917 retain tumor specific colonization properties but show less side effects in immunocompetent mice.

For intravenous injection a preferred dose of bacteria is the dose in which the greatest number of bacteria is found in the tumor and the lowest amount found in other tissues. In mice, Stritzker et al (International Journal of Medical Microbiology 297 (2007) 151-162; Tumor specific colonization, tissue distribution, and gene induction by Escherichia coli Nissle 1917 in live mice) found that the lowest number of bacteria needed for successful tumor colonization was 2e4 CFU, in which half of the mice showed tumor colonization. Injection of 2e5 and 2e6 CFU resulted in colonization of all tumors, and numbers of bacteria in the tumors increased. However, at higher concentrations, bacterial counts became detectable in the liver and the spleen.

In some embodiments, the microorganisms of the disclosure may be administered orally. In some embodiments, the genetically engineered bacteria may be useful in the prevention, treatment or management of liver cancer or liver metastases. For example, Danino et al showed that orally administered E. coli Nissle is able to colonize liver metastases by crossing the gastrointestinal tract in a mouse model of liver metastases (Danino et al., Programmable probiotics for detection of cancer in urine. Science Translational Medicine, 7 (289): 1-10, the contents of which is herein incorporated by reference in its entirety).

In one embodiment, the genetically engineered microorganism is delivered by intratumor injection. In one embodiment, the genetically engineered microorganisms is delivered intrapleurally. In one embodiment, the genetically engineered microorganism is delivered subcutaneously. In one embodiment, the genetically engineered microorganism is delivered intravenously. In one embodiment, the genetically engineered microorganism is delivered intrapleurally.

In some embodiments, the genetically engineered microorganisms of the invention may be administered intratumorally according to a regimen which requires multiple injections. In some embodiments, the same bacterial strains are administered in each intratumoral injection. In some embodiments, a first strain is injected first and a second strain is injected at a later timepoint. For example, a strain capable of producing an immune initiator, e.g., STING agonist, may be administered concurrently or sequentially with a strain capable or producing another immune initiator, e.g., a co-stimulatory molecule, e.g., agonistic anti-OX40, 41BB, or GITR. Additional injections of the two immune initiators, either concurrently or sequentially, can follow. In another example, a strain capable of producing an immune initiator, e.g., STING agonist, may be administered first, and a strain capable of producing an immune sustainer, e.g., kynurenine consumption, or anti-PD-1/anti-PD-L1 secretion or anti-PD-1/anti-PD-L1 surface display, may be administered second. Additional injections of STING agonist producing strains and/or anti-PD-1/anti-PD-L1 producing strains can follow. In any of these examples, optionally, a tumor biopsy may be taken concurrently with the intratumor injection. Optionally, antibiotics can be used to clear a first strain from the tumor before injection of a second strain. Alternatively, an auxotrophic modification, e.g., mutation in the dapA gene, which limits colonization, can be incorporated into the first strain, which may eliminate the bacteria of the first strain prior to injection of a second strain.

Tumor types into which the engineered bacteria or virus of the current invention are intratumorally delivered include locally advanced and metastatic tumors, including but not limited to, B, T, and NK cell lymphomas, colon and rectal cancers, melanoma, including metastatic melanoma, mycosis fungoides, Merkel carcinoma, liver cancer, including hepatocellular carcinoma and liver metastasis secondary to colorectal cancer, pancreatic cancer, breast cancer, follicular lymphoma, prostate cancer, refractory liver cancer, and Merkel cell carcinoma.

In some embodiments, tumor cell lysis occurs as part of the intratumor injection. As result, tumor antigens may exposed eliciting an anti-tumor response. This exposure may work together with the effector expressed by the bacteria to enhance the anti-tumor effect. In some embodiments, tumor cell lysis does not occur as part of the intratumor injection.

The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinized maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered bacteria are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionized at low pH, and therefore remain insoluble. But as the pH increases in the gastrointestinal tract, the acidic functional groups are capable of ionization, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (M H EC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered microorganisms and composition thereof is formulated for intravenous administration, intratumor administration, or peritumor administration. The genetically engineered microorganisms may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, the genetically engineered OVs are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Methods of Treatment

Another aspect of the invention provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

The method may comprise preparing a pharmaceutical composition with at least one genetically engineered species, strain, or subtype of bacteria described herein, and administering the pharmaceutical composition to a subject in a therapeutically effective amount. The genetically engineered microorganisms may be administered locally, e.g., intratumorally or peritumorally into a tissue or supplying vessel, or systemically, e.g., intravenously by infusion or injection. In some embodiments, the genetically engineered bacteria are administered intravenously, intratumorally, intra-arterially, intramuscularly, intraperitoneally, orally, or topically. In some embodiments, the genetically engineered microorganisms are administered intravenously, i.e., systemically.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions of the invention to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

For genetically engineered microorganisms expressing immune-based immune modulators, responses patterns may be different than for traditional cytotoxic therapies. For example, tumors treated with immune-based therapies may enlarge before they regress, and/or new lesions may appear (Agarwala et al., 2015). Increased tumor size may be due to heavy infiltration with lymphocytes and macrophages that are normally not present in tumor tissue. Additionally, response times may be slower than response times associated with standard therapies, e.g., cytotoxic therapies. In some embodiments, delivery of the immune modulator may modulate the growth of a subject's tumor and/or ameliorate the symptoms of a cancer while temporarily increasing the volume and/or size of the tumor.

The genetically engineered bacteria may be destroyed, e.g., by defense factors in tissues or blood serum (Sonnenborn et al., 2009), or by activation of a kill switch, several hours or days after administration. Thus, the pharmaceutical composition comprising the gene or gene cassette for producing the immune modulator may be re-administered at a therapeutically effective dose and frequency. In alternate embodiments, the genetically engineered bacteria are not destroyed within hours or days after administration and may propagate in the tumor and colonize the tumor.

The pharmaceutical composition may be administered alone or in combination with one or more additional therapeutic agents, e.g., a chemotherapeutic drug or a checkpoint inhibitor, e.g., as described herein and known in the art. An important consideration in selecting the one or more additional therapeutic agents is that the agent(s) should be compatible with the genetically engineered bacteria of the invention, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In some embodiments, the pharmaceutical composition comprising the bacteria augments the effect of a checkpoint inhibitor or a chemotherapeutic agent, e.g., allowing lowering of a the dose of systemically administrated chemotherapeutic or immunotherapeutic agents. In some embodiments, the pharmaceutical composition is administered with one or more commensal or probiotic bacteria, e.g., Bifidobacterium or Bacteroides.

In certain embodiments, the pharmaceutical composition may be administered to a subject for treating cancer by administering a first genetically engineered bacterium to the subject, wherein the first genetically engineered bacterium comprises at least one gene encoding a first immune initiator; and administering a second genetically engineered bacterium to the subject, wherein the second genetically engineered bacterium comprising at least one gene encoding a second immune initiator. In some embodiments, the administering steps are performed at the same time. In some embodiments, administering the first genetically engineered bacterium to the subject occurs before the administering of the second genetically engineered bacterium to the subject. In some embodiments, administering of the second genetically engineered bacterium to the subject occurs before the administering of the first genetically engineered bacterium to the subject. In some embodiments, the ratio of the first genetically engineered bacterium to the second genetically engineered bacterium is 5:1, 4:1, 3:1, 2:1, 1:1, 1:2, 1:3, 1:4, or 1:5. In some embodiments, the ratio of the second genetically engineered bacterium to the first genetically engineered bacterium is 5:1, 4:1, 3:1, 2:1, 1:1, 1:2, 1:3, 1:4, or 1:5.

### Chemotherapeutic Agents

In some embodiments, the genetically engineered bacteria are administered sequentially, simultaneously, or subsequently to dosing with one or more chemotherapeutic agents. In some embodiments, the genetically engineered bacteria are administered sequentially, simultaneously, or subsequently to dosing with one or more chemotherapeutic agents selected from Trabectedin®, Belotecan®, Cisplatin®, Carboplatin®, Bevacizumab®, Pazopanib®, 5-Fluorouracil, Capecitabine®, Irinotecan®, and Oxaliplatin®. In some embodiments, the genetically engineered bacteria are administered sequentially, simultaneously, or subsequently to dosing with gemcitabine (Gemzar). In some embodiments, the genetically engineered bacteria are administered sequentially, simultaneously, or subsequently to dosing with cyclophosphamide. In any of these embodiments, the one or more bacteria are administered systemically or orally or intratumorally.

In some embodiments, one or more engineered bacteria described herein which comprise gene sequence(s) encoding enzymes for the production or consumption of a metabolite, e.g., kynurenine or adenosine consumers ammonia consumers, arginine producers and/or STING agonist producers, are administered sequentially, simultaneously, or subsequently to dosing with one or more chemotherapeutic agents. In some embodiments, the chemotherapeutic agent is administered systemically, and the bacteria are administered intratumorally. In some embodiments, the chemotherapeutic agent and bacteria are administered systemically. In some embodiments, one or more engineered bacteria described herein, which comprise gene sequence(s) encoding a metabolic converter, e.g., kynurenine, consumers, adenosine consumers, arginine producers, ammonia consumers and/or STING agonist producers and/or ammonia consumers, are administered sequentially, simultaneously, or subsequently to dosing with a chemotherapeutic agent. In one embodiment, the chemotherapeutic agent is cyclophosphamide.

In some embodiments, one or more engineered bacteria expressing any one or more of the described circuits for the degradation of adenosine are administered sequentially, simultaneously, or subsequently to dosing a chemotherapeutic agent. In one embodiment, the chemotherapeutic agent is cyclophosphamide. In some embodiments, genetically engineered bacteria expressing any one or more of the described circuits for the consumption of kynurenine are administered sequentially, simultaneously, or subsequently to dosing with a chemotherapeutic agent. In one embodiment, the chemotherapeutic agent is cyclophosphamide. In some embodiments, genetically engineered bacteria expressing any one or more of the described circuits for the production of arginine are administered sequentially, simultaneously, or subsequently to dosing with a chemotherapeutic agent. In some embodiments, genetically engineered bacteria expressing any one or more of the described circuits for the consumption and/or arginine production are administered sequentially, simultaneously, or subsequently to dosing with a chemotherapeutic agent. In one embodiment, the chemotherapeutic agent is cyclophosphamide.

In some embodiments, the one or more engineered bacteria, e.g., adenosine consumer, kynurenine consumer, ammonia consumer, arginine producer, are able to improve anti-tumor activity (e.g., tumor proliferation, size, volume, weight) of the co-administered chemotherapeutic agent (e.g., cyclophosphamide or another agent described herein or known in the art), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions or as compared to an unmodified bacteria of the same subtype under the same conditions. In some embodiments, the genetically engineered bacteria, e.g., adenosine consumer, kynurenine consumer, arginine producer, are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the genetically engineered bacteria, e.g., adenosine consumer, kynurenine consumer, arginine producer, are able to improve anti-tumor activity (e.g., tumor proliferation, size, volume, weight) of the co-administered chemotherapeutic agent (e.g., cyclophosphamide or another agent described herein or known in the art), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, one or more engineered bacteria described herein, which comprise gene sequence(s) for the production of an effector for immune activation and priming, are administered sequentially, simultaneously, or subsequently to dosing with a chemotherapeutic agent. In some embodiments, the chemotherapeutic agent is administered systemically, and the bacteria are administered intratumorally. In some embodiments, the chemotherapeutic agent and bacteria are administered systemically.

In some embodiments, the genetically engineered bacteria administered with the chemotherapeutic agent comprise gene sequence(s) encoding one or more enzymes for the production of one or more STING agonists. In some embodiments, one or more engineered bacteria described herein, which comprise gene sequence(s) encoding diadenylate cyclase and/or other STING agonist producing enzyme, such as human or bacterial cGAS, are administered sequentially, simultaneously, or subsequently to dosing with a chemotherapeutic agent. In one embodiment, the chemotherapeutic agent is cyclophosphamide.

In some embodiments, the genetically engineered bacteria expressing diadenylate cyclase, human or bacterial cGAS, and/or or other enzyme for the generation of STING agonist, are able to improve anti-tumor activity (e.g., tumor proliferation, size, volume, weight) of the co-administered chemotherapeutic agent (e.g., cyclophosphamide), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions or as compared to an unmodified bacteria of the same subtype under the same conditions. In some embodiments, the genetically engineered bacteria expressing diadenylate cyclase, cGAS, and/or other enzyme for the generation of STING agonist, are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the genetically engineered bacteria expressing diadenylate cyclase or other enzyme for the generation of STING agonist, are able to improve anti-tumor activity (e.g., tumor proliferation, size, volume, weight) of the co-administered chemotherapeutic agent (e.g., cyclophosphamide or another agent described herein or known in the art), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, genetically engineered bacteria expressing any one or more of the described cytosine deaminases for the conversion of 5-FC to 5-FU are administered sequentially, simultaneously, or subsequently to dosing a chemotherapeutic agent. In one embodiment, the chemotherapeutic agent is cyclophosphamide.

In some embodiments, the genetically engineered bacteria administered with the chemotherapeutic agent comprise gene sequence(s) encoding one or more enzymes for the conversion of 5-FC to 5-FU. In some embodiments, the genetically engineered bacteria expressing cytosine deaminase for the conversion of 5-FC to 5-FU, are able to improve anti-tumor activity (e.g., tumor proliferation, size, volume, weight) of the co-administered chemotherapeutic agent (e.g., cyclophosphamide), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions or as compared to an unmodified bacteria of the same subtype under the same conditions.

In some embodiments, the genetically engineered bacteria expressing cytosine deaminase for the conversion of 5-FC to 5-FU, are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the genetically engineered bacteria expressing cytosine deaminase for the conversion of 5-FC to 5-FU, are able to improve anti-tumor activity (e.g., tumor proliferation, size, volume, weight) of the co-administered chemotherapeutic agent (e.g., cyclophosphamide or another agent described herein or known in the art), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, one or more engineered bacteria described herein which comprise gene sequence(s) encoding enzymes for the production of a STING agonist in combination with gene sequence(s) encoding one or more enzymes for the degradation of adenosine, for kynurenine consumption, for ammonia consumption and/or arginine production, are administered sequentially, simultaneously, or subsequently to dosing with one or more chemotherapeutic reagents described herein. In any of these embodiments, the one or more chemotherapeutic reagent is administered systemically or orally or intratumorally. In any of these embodiments, the one or more bacteria are administered systemically or orally or intratumorally.

In some embodiments, in which one or more genetically engineered bacteria are combined with a chemotherapeutic agent, the genetically engineered bacteria are able to improve anti-tumor activity (e.g., tumor proliferation, size, volume, weight) of the co-administered chemotherapeutic agent (e.g., cyclophosphamide), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions or as compared to an unmodified bacteria of the same subtype under the same conditions. In some embodiments, one or more genetically engineered bacteria are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, one or more genetically engineered bacteria are able to improve anti-tumor activity (e.g., tumor proliferation, size, volume, weight) of the co-administered chemotherapeutic agent (e.g., cyclophosphamide or another agent described herein or known in the art), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, one or more genetically engineered bacteria comprising gene sequence(s) encoding one or more adenosine degradation enzyme(s) described herein are administered sequentially, simultaneously, or subsequently to dosing with gemcitabine. In some embodiments, the enzymes for adenosine consumption encoded by the genetically engineered bacteria comprise one or more of add, xapA, deoD, xdhA, xdhB, and xdhC and nupC. In some embodiments, the enzymes for adenosine consumption encoded by the genetically engineered bacteria comprise one or more of add, xapA, deoD, xdhA, xdhB, and xdhC and nupG. In some embodiments, one or more engineered bacteria described herein which comprise gene sequence(s) encoding anti-CD40 antibody for surface display or secretion, are administered sequentially, simultaneously, or subsequently to dosing with gemcitabine. In some embodiments, one or more engineered bacteria described herein which comprise gene sequence(s) encoding hyaluronidase for secretion or for surface display, are administered sequentially, simultaneously, or subsequently to dosing with gemcitabine. In any of these embodiments, the one or more bacteria are administered systemically or orally or intratumorally.

In some embodiments, in which the genetically engineered bacteria are combined with a gemcitabine, the genetically engineered bacteria are able to improve anti-tumor activity (e.g., tumor proliferation, size, volume, weight) of the co-administered chemotherapeutic agent (e.g., cyclophosphamide), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions or as compared to an unmodified bacteria of the same subtype under the same conditions. In some embodiments, the genetically engineered bacteria are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the genetically engineered bacteria are able to improve anti-tumor activity (e.g., tumor proliferation, size, volume, weight) of the co-administered chemotherapeutic agent (e.g., cyclophosphamide or another agent described herein or known in the art), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

### Checkpoint Inhibition

In certain embodiments, one or more genetically engineered bacteria are administered sequentially, simultaneously, or subsequently to dosing with one or more checkpoint inhibitors, immune stimulatory antibodies (inhibitory or agonistic) or other agonists known in the art or described herein. In certain embodiments, the one or more genetically engineered bacteria are administered sequentially, simultaneously, or subsequently to dosing with one checkpoint inhibitors, immune stimulatory antibodies (inhibitory or agonistic) or other agonists known in the art or described herein. In certain embodiments, the one or more genetically engineered bacteria are administered sequentially, simultaneously, or subsequently to dosing with two checkpoint inhibitors, immune stimulatory antibodies (inhibitory or agonistic) or other agonists known in the art or described herein.

Non-limiting examples of immune checkpoint inhibitors include CTLA-4 antibodies (including but not limited to Ipilimumab and Tremelimumab (CP675206)), anti-4-1BB (CD137, TNFRSF9) antibodies (including but not limited to PF-05082566, and Urelumab), anti CD134 (OX40) antibodies, including but not limited to Anti-OX40 antibody (Providence Health and Services), anti-PD-1 antibodies (including but not limited to Nivolumab, Pidilizumab, Pembrolizumab (MK-3475/SCH900475, lambrolizumab, REGN2810, PD-1 (Agenus)), anti-PD-L1 antibodies (including but not limited to durvalumab (MEDI4736), avelumab (MSB0010718C), and atezolizumab (MPDL3280A, RG7446, R05541267)), and anti-KIR antibodies (including but not limited to Lirilumab), LAG3 antibodies (including but not limited to BMS-986016), anti-CCR4 antibodies (including but not limited to Mogamulizumab), anti-CD27 antibodies (including but not limited to Varlilumab), anti-CXCR4 antibodies (including but not limited to Ulocuplumab). In some embodiments, the at least one bacterial cell is administered sequentially, simultaneously, or subsequently to dosing with an anti-phosphatidyl serine antibody (including but not limited to Bavituxumab).

In some embodiments, the at least one bacterial cell is administered sequentially, simultaneously, or subsequently to dosing with one or more antibodies selected from TLR9 antibody (including, but not limited to, MGN1703 PD-1 antibody (including, but not limited to, SHR-1210 (Incyte/Jiangsu Hengrui)), anti-OX40 antibody (including, but not limited to, OX40 (Agenus)), anti-Tim3 antibody (including, but not limited to, Anti-Tim3 (Agenus/INcyte)), anti-Lag3 antibody (including, but not limited to, Anti-Lag3 (Agenus/INcyte)), anti-B7H3 antibody (including, but not limited to, Enoblituzumab (MGA-271), anti-CT-011 (hBAT, hBAT1) as described in WO2009101611, anti-PDL-2 antibody (including, but not limited to, AMP-224 (described in WO2010027827 and WO2011066342)), anti-CD40 antibody (including, but not limited to, CP-870, 893), anti-CD40 antibody (including, but not limited to, CP-870, 893).

In some embodiments, one or more engineered bacteria described herein, which comprise gene sequence(s) encoding enzymes for the production or consumption of metabolite, e.g., kynurenine, consumers, adenosine consumers, arginine producers, ammonia consumers and/or STING agonist producers are administered sequentially, simultaneously, or subsequently to dosing with one or more checkpoint inhibitors. In some embodiments, the checkpoint inhibitor is administered systemically, and the bacteria are administered intratumorally. In some embodiments, the checkpoint inhibitor and bacteria are administered systemically. In some embodiments, one or more engineered bacteria described herein, which comprise gene sequence(s) encoding a metabolic converter, e.g., kynurenine consumers, adenosine consumers, arginine producers and/or ammonia consumers, and/or STING agonist producers, are administered sequentially, simultaneously, or subsequently to dosing with an anti-PD1 antibody. In some embodiments, one or more engineered bacteria described herein, which comprise gene sequence(s) encoding a metabolic converter, e.g., kynurenine, consumers, adenosine consumers, arginine producers, ammonia consumers and/or STING agonist producers, are administered sequentially, simultaneously, or subsequently to dosing with an anti-CTLA-4 antibody. In some embodiments, genetically engineered bacteria expressing any one or more of the described circuits for the degradation of adenosine are administered sequentially, simultaneously, or subsequently to dosing with an anti-PD-L1 antibody.

In some embodiments, genetically engineered bacteria comprising gene sequence(s) encoding a metabolic converter, e.g., kynurenine, consumers, adenosine consumers, arginine producers, ammonia consumers and/or STING agonist producers, are administered sequentially, simultaneously, or subsequently to dosing with an anti-CTLA4 antibody and an anti-PD-1 antibody. In some embodiments, genetically engineered bacteria expressing any one or more of the described circuits for the degradation of adenosine are administered sequentially, simultaneously, or subsequently to dosing with an anti-PD-L1 antibody and a CTLA4 antibody.

In some embodiments, genetically engineered bacteria expressing any one or more of the described circuits for the degradation of adenosine are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, genetically engineered bacteria expressing any one or more of the described circuits for the degradation of adenosine are administered sequentially, simultaneously, or subsequently to dosing with anti-PD1 antibody. In some embodiments, genetically engineered bacteria expressing any one or more of the described circuits for the degradation of adenosine are administered sequentially, simultaneously, or subsequently to dosing with anti-PD-L1 antibody. In some embodiments, genetically engineered bacteria expressing any one or more of the described circuits for the degradation of adenosine are administered sequentially, simultaneously, or subsequently to dosing with anti-CTLA4 antibody. In some embodiments, genetically engineered bacteria of expressing any one or more of the described circuits for the degradation of adenosine are administered sequentially, simultaneously, or subsequently to dosing with an anti-CTLA4 antibody and anti-PD1 antibody and/or PD-L1 antibody.

In some embodiments, genetically engineered bacteria expressing any one or more of the described circuits for the consumption of kynurenine are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, genetically engineered bacteria expressing any one or more of the described circuits for the consumption of kynurenine are administered sequentially, simultaneously, or subsequently to dosing with an anti-CTLA4 antibody. In some embodiments, genetically engineered bacteria expressing any one or more of the described circuits for the consumption of kynurenine are administered sequentially, simultaneously, or subsequently to dosing with anti-PD1 antibody. In some embodiments, genetically engineered bacteria expressing any one or more of the described circuits for the consumption of kynurenine are administered sequentially, simultaneously, or subsequently to dosing with anti-PD-L1 antibody. In some embodiments, genetically engineered bacteria of expressing any one or more of the described circuits for the consumption of kynurenine are administered sequentially, simultaneously, or subsequently to dosing with an anti-CTLA4 antibody and anti-PD1 antibody and/or anti-PD-L1 antibody.

In some embodiments, genetically engineered bacteria expressing any one or more of the described circuits for the production of arginine and/or consumption of ammonia are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, genetically engineered bacteria expressing any one or more of the described circuits for the production of arginine and/or consumption of ammonia are administered sequentially, simultaneously, or subsequently to dosing with an anti-CTLA4 antibody. In some embodiments, genetically engineered bacteria expressing any one or more of the described circuits for the production of arginine and/or consumption of ammonia are administered sequentially, simultaneously, or subsequently to dosing with anti-PD1 antibody. In some embodiments, genetically engineered bacteria expressing any one or more of the described circuits for the production of arginine and/or consumption of ammonia are administered sequentially, simultaneously, or subsequently to dosing with anti-PD-1L antibody. In some embodiments, genetically engineered bacteria of expressing any one or more of the described circuits for the production of arginine and/or consumption of ammonia are administered sequentially, simultaneously, or subsequently to dosing with an anti-CTLA4 antibody and anti-PD1 antibody.

In some embodiments, genetically engineered bacteria expressing any one or more of the described circuits for the production of one or more STING agonists are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, genetically engineered bacteria expressing any one or more of the described circuits for the production of one or more STING agonists are administered sequentially, simultaneously, or subsequently to dosing with an anti-CTLA4 antibody. In some embodiments, genetically engineered bacteria expressing any one or more of the described circuits for the production of one or more STING agonists are administered sequentially, simultaneously, or subsequently to dosing with anti-PD1 antibody. In some embodiments, genetically engineered bacteria expressing any one or more of the described circuits for the production of one or more STING agonists are administered sequentially, simultaneously, or subsequently to dosing with anti-PD-L1 antibody. In some embodiments, genetically engineered bacteria of expressing any one or more of the described circuits for the production of one or more STING agonists are administered sequentially, simultaneously, or subsequently to dosing with an anti-CTLA4 antibody and anti-PD1 antibody and/or anti-PD-L1 antibody.

In some embodiments, one or more genetically engineered bacteria expressing any one or more of the described circuits for the production of one or more STING agonists and one or more metabolic conversion circuits are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, one or more genetically engineered bacteria expressing any one or more of the described circuits for the production of one or more STING agonists and one or more metabolic conversion circuits are administered sequentially, simultaneously, or subsequently to dosing with an anti-CTLA-4 antibody. In some embodiments, one or more genetically engineered bacteria expressing any one or more of the described circuits for the production of one or more STING agonists and one or more metabolic conversion circuits are administered sequentially, simultaneously, or subsequently to dosing with anti-PD-1 antibody. In some embodiments, one or more genetically engineered bacteria expressing any one or more of the described circuits for the production of one or more STING agonists and one or more metabolic conversion circuits are administered sequentially, simultaneously, or subsequently to dosing with anti-PD-L1 antibody. In some embodiments, genetically engineered bacteria of expressing any one or more of the described circuits for the production of one or more STING agonists and one or more metabolic conversion circuits are administered sequentially, simultaneously, or subsequently to dosing with an anti-CTLA-4 antibody and anti-PD-1 antibody and/or anti-PD-L1 antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions in dapA. In any of these embodiments, the bacteria may comprise one or more auxotrophic modifications, e.g., mutations or deletions in thyA. In any of these embodiments, the bacteria may further comprise a phage modification, e.g., a mutation or deletion, in an endogenous prophage as described herein.

In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with an anti-CTLA-4 antibody. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with anti-PD-1 antibody. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with anti-PD-L1 antibody.

In some embodiments, genetically engineered bacteria of expressing any one or more of the described circuits for the production of one or more STING agonists and one or more kynureninase(s), e.g., from Pseudomonas fluorescens, are administered sequentially, simultaneously, or subsequently to dosing with an anti-CTLA-4 antibody and anti-PD-1 antibody and/or anti-PD-L1 antibody. In some embodiments, the STING agonist is c-diAMP. In some embodiments, the STING agonist producing enzyme is DacA, e.g., from Listeria monocytogenes. In certain embodiments, dacA is operably linked to a promoter inducible under low oxygen conditions, e.g., a FNR promoter. In some embodiments, kynureninase is from Pseudomonas fluorescens and the bacterium comprising gene sequences encoding kynureninase further comprises a mutation or deletion in trpE. In some embodiments, kynureninase is operably linked to a constitutive promoter In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In certain embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with a anti-PD-1, anti-PD-L1 or anti-CTLA-4. In one embodiment, one or more genetically engineered bacteria comprise gene sequence(s) encoding DacA, e.g., from Listeria monocytogenes, wherein DacA is operably linked to a promoter inducible under low oxygen conditions, e.g., a FNR promoter. The bacteria comprising gene sequences encoding dacA comprise a mutation or deletion in dapA. The one or more genetically engineered bacteria further comprise gene sequences encoding kynureninase from Pseudomonas fluorescens and the bacterium comprising gene sequences encoding kynureninase further comprises a mutation or deletion in trpE. In certain embodiments, dacA and kynureninase sequences are integrated into the bacterial chromosome. In one specific embodiment, the one or more genetically engineered bacteria may further comprise mutation(s) or deletion(s) in thyA. In one specific embodiment, the bacteria may further comprise a phage modification, e.g., a mutation or deletion, in an endogenous prophage as described herein.

In one specific embodiment, the checkpoint inhibitor is PD-1. In one specific embodiment, the checkpoint inhibitor is PD-L1. In one specific embodiment, the checkpoint inhibitor is CTLA-4.

In one embodiment, dacA circuitry (e.g., from Listeria monocytogenes, e.g., under control of a low oxygen promoter and chromosomally integrated), kynureninase circuitry (e.g., from Pseudomonas fluorescens, e.g., under the control of a constitutive promoter and chromosomally integrated) and auxotrophic mutations (mutations or deletions in trpE, dapA, and thyA) are combined in one bacterium. In an alternate embodiment, a bacterial composition comprises a first bacterium comprising gene sequences encoding dacA (e.g., from Listeria monocytogenes, e.g., under control of a low oxygen promoter and chromosomally integrated), further comprising a mutation or deletion in dapA, and with an optional mutations or deletions in thyA and a second bacterium comprising gene sequences encoding kynureninase circuitry (e.g., from Pseudomonas fluorescens, e.g., under the control of a constitutive promoter and chromosomally integrated), a mutation or deletion in trpE, and optionally a mutation or deletion in thyA. In any of these embodiments, the bacteria may further comprise a phage modification, e.g., a mutation or deletion, in an endogenous prophage as described herein.

In some embodiments, one or more genetically engineered bacteria expressing any one or more of the described circuits for the production of one or more STING agonists and one or more adenosine consuming enzymes described herein are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, one or more genetically engineered bacteria expressing any one or more of the described circuits for the production of one or more STING agonists and one or more adenosine consuming enzymes described herein are administered sequentially, simultaneously, or subsequently to dosing with an anti-CTLA-4 antibody. In some embodiments, one or more genetically engineered bacteria expressing any one or more of the described circuits for the production of one or more STING agonists and one or more adenosine consuming enzymes described herein are administered sequentially, simultaneously, or subsequently to dosing with anti-PD-1 antibody. In some embodiments, one or more genetically engineered bacteria expressing any one or more of the described circuits for the production of one or more STING agonists and one or more adenosine consuming enzymes described herein are administered sequentially, simultaneously, or subsequently to dosing with anti-PD-L1 antibody. In some embodiments, genetically engineered bacteria of expressing any one or more of the described circuits for the production of one or more STING agonists and one or more adenosine consuming enzymes described herein are administered sequentially, simultaneously, or subsequently to dosing with an anti-CTLA-4 antibody and anti-PD-1 antibody and/or anti-PD-L1 antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria comprising any one or more of the described circuits for the production of one or more STING agonists and one or more arginine production and/or ammonia consuming circuits described herein are administered sequentially, simultaneously, or subsequently to dosing with a checkpoint inhibitor, e.g., as described herein and known in the art, including but not limited to anti-PD-1, anti-PD-L1, and anti-CTLA-4. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with an anti-CTLA-4 antibody. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with anti-PD-1 antibody. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with anti-PD-L1 antibody. In some embodiments, genetically engineered bacteria of comprising any one or more of the described circuits for the production of one or more STING agonists and one or more arginine production and/or ammonia consuming circuits described herein are administered sequentially, simultaneously, or subsequently to dosing with an anti-CTLA-4 antibody and anti-PD-1 antibody and/or anti-PD-L1 antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, genetically engineered bacteria of expressing any one or more of the described circuits for the production of one or more STING agonists and one or more arginine producing/ammonia consuming circuits, are administered sequentially, simultaneously, or subsequently to dosing with an anti-CTLA-4 antibody and anti-PD-1 antibody and/or anti-PD-L1 antibody. In some embodiments, the STING agonist is c-diAMP. In some embodiments, the STING agonist producing enzyme is DacA, e.g., from Listeria monocytogenes. In certain embodiments, dacA is operably linked to a promoter inducible under low oxygen conditions, e.g., a FNR promoter. In some embodiments, the arginine producing/ammonia consuming circuit comprises feedback-resistant ArgA and the bacterium comprising gene sequences encoding feedback-resistant ArgA further has a mutation or deletion in ArgR In some embodiments, feedback-resistant ArgA is operably linked to a promoter inducible under low oxygen conditions. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, thyA.

In certain embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with a anti-PD-1, anti-PD-L1 or anti-CTLA-4. In one embodiment, one or more genetically engineered bacteria comprise gene sequence(s) encoding DacA, e.g., from Listeria monocytogenes, wherein DacA is operably linked to a promoter inducible under low oxygen conditions, e.g., a FNR promoter. The bacteria comprising gene sequences encoding dacA comprise a mutation or deletion in dapA. The one or more genetically engineered bacteria further comprise gene sequences encoding feedback-resistant ArgA and the bacterium comprising gene sequences encoding feedback-resistant ArgA further comprises a mutation or deletion in ArgR. In certain embodiments, dacA and feedback-resistant ArgA sequences are integrated into the bacterial chromosome. In one specific embodiment, the one or more genetically engineered bacteria may further comprise mutation(s) or deletion(s) in thyA. In one specific embodiment, the checkpoint inhibitor is PD-1. In one specific embodiment, the checkpoint inhibitor is PD-L1. In one specific embodiment, the checkpoint inhibitor is CTLA-4.

In one embodiment, dacA circuitry (e.g., from Listeria monocytogenes, e.g., under control of a low oxygen promoter and chromosomally integrated), arginine production/ammonia consumption circuitry (e.g., comprising ArgAfbr, e.g., under the control of a low oxygen inducible promoter and chromosomally integrated and ΔArgR) and auxotrophic mutations (mutations or deletions in dapA, and thyA) are combined in one bacterium. In an alternate embodiment, a bacterial composition comprises a first bacterium comprising gene sequences encoding dacA (e.g., from Listeria monocytogenes, e.g., under control of a low oxygen promoter and chromosomally integrated), further comprising a mutation or deletion in dapA, and with an optional mutation or deletion in thyA, and a second bacterium comprising gene sequences encoding arginine production/ammonia consumption circuitry (e.g., comprising ArgAfbr, e.g., under the control of a low oxygen inducible promoter and chromosomally integrated and ΔArgR), and optionally a mutation or deletion in thyA.

In some embodiments, one or more genetically engineered bacteria, e.g., adenosine consumer, kynurenine consumer, ammonia consumer, arginine producer, and/or STING agonist producer are able to improve anti-tumor activity (e.g., tumor proliferation, size, volume, weight) of the co-administered checkpoint inhibitors (e.g., anti-PD-1, anti-PD-L1 and/or anti-CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to an unmodified bacteria of the same subtype under the same conditions. In some embodiments, one or more genetically engineered bacteria, e.g., adenosine consumer, kynurenine consumer, ammonia consumer, arginine producer, and/or STING agonist producer, are able to improve anti-tumor activity (e.g., tumor proliferation, size, volume, weight) of the co-administered checkpoint inhibitors (e.g., anti-PD-1, anti-PD-L1 and/or anti-CTLA-4), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, one or more genetically engineered bacteria, e.g., adenosine consumer, kynurenine consumer, ammonia consumer, arginine producer, and/or STING agonist producer, are able to improve anti-tumor activity (e.g., tumor proliferation, size, volume, weight) of the co-administered checkpoint inhibitor(e.g., anti-PD-1, anti-PD-L1 and/or anti-CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to consume adenosine are able to improve anti-tumor activity (e.g., tumor proliferation, size, volume, weight) of the co-administered checkpoint inhibitors(e.g., PD-1 and/or CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to an unmodified bacteria of the same subtype under the same conditions. In some embodiments, the bacteria genetically engineered to consume adenosine are able to improve anti-tumor activity (e.g., tumor proliferation, size, volume, weight) of the co-administered checkpoint inhibitors (e.g., anti-PD-1, anti-PD-L1 and/or anti-CTLA-4), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to consume adenosine are able to improve anti-tumor activity (e.g., tumor proliferation, size, volume, weight) of the co-administered checkpoint inhibitor(e.g., anti-PD-1, anti-PD-L1 and/or anti-CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to consume kynurenine are able to improve anti-tumor activity (e.g., tumor proliferation, size, volume, weight) of the co-administered checkpoint inhibitors (e.g., anti-PD-1, anti-PD-L1 and/or anti-CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to an unmodified bacteria of the same subtype under the same conditions. In some embodiments, the bacteria genetically engineered to consume kynurenine are able to improve anti-tumor activity (e.g., tumor proliferation, size, volume, weight) of the co-administered checkpoint inhibitors (e.g., PD-1, PD-L1 and/or CTLA-4), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to consume kynurenine are able to improve anti-tumor activity (e.g., tumor proliferation, size, volume, weight) of the co-administered checkpoint inhibitor(e.g., PD-1, PD-L1 and/or CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to produce arginine and/or consume ammonia are able to improve anti-tumor activity (e.g., tumor proliferation, size, volume, weight) of the co-administered checkpoint inhibitors(e.g., PD-1, PD-L1 and/or CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to an unmodified bacteria of the same subtype under the same conditions. In some embodiments, the bacteria genetically engineered to produce arginine and/or consume ammonia are able to improve anti-tumor activity (e.g., tumor proliferation, size, volume, weight) of the co-administered checkpoint inhibitors (e.g., PD-1, PD-L1 and/or CTLA-4), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to produce arginine and/or consume ammonia are able to improve anti-tumor activity (e.g., tumor proliferation, size, volume, weight) of the co-administered checkpoint inhibitor (e.g., PD-1, PD-L1 and/or CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to produce STING agonist(s) are able to improve anti-tumor activity (e.g., tumor proliferation, size, volume, weight) of the co-administered checkpoint inhibitors(e.g., anti-PD-1, anti-PD-L1 and/or anti-CTLA-4), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a checkpoint inhibitor therapy alone under the same conditions or as compared to an unmodified bacteria of the same subtype under the same conditions. In some embodiments, the bacteria genetically engineered to produce STING agonist(s) are able to improve anti-tumor activity (e.g., tumor proliferation, size, volume, weight) of the co-administered checkpoint inhibitors (e.g., PD-1, PD-L1 and/or CTLA-4), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to produce STING agonist(s) are able to improve anti-tumor activity (e.g., tumor proliferation, size, volume, weight) of the co-administered checkpoint inhibitor(e.g., PD-1, PD-L1 and/or CTLA-4), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

### Co-Stimulatory Molecules

In certain embodiments, one or more genetically engineered bacteria are administered sequentially, simultaneously, or subsequently to dosing with one or more agonistic immune stimulatory molecules or agonists, including but not limited to, agonistic antibodies.

In some embodiments, the at least one bacterial cell is administered sequentially, simultaneously, or subsequently to dosing with one or more antibodies selected from anti-OX40 antibody (including, but not limited to, INCAGN01949 (Agenus); BMS 986178 (Bristol-Myers Squibb), MEDI0562 (Medimmune), GSK3174998 (GSK), PF-04518600 (Pfizer)), anti-41BB/CD137 (including but not limited to PF-05082566 (Pfizer), urelumab (BMS-663513; Bristol-Myers Squibb), and anti-GITR (including but not limited to TRX518 (Leap Therapeutics), MK-4166 (Merck), MK-1248 (Merck), AMG 228 (Amgen), BMS-986156 (BMS), INCAGN01876 (Incyte/Agenus), MEDI1873 (AZ), GWN323 (NVS). In some embodiments, one or more engineered bacteria described herein, which comprise gene sequence(s) encoding enzymes for the production or consumption of metabolite, e.g., kynurenine, consumers, adenosine consumers, arginine producers, ammonia consumers and/or STING agonist producers are administered sequentially, simultaneously, or subsequently to dosing with agonistic antibodies selected from anti-OX40 antibody, anti-41BB, and/or anti-GITR. In some embodiments, the agonistic antibody, e.g., anti-OX40 antibody, anti-41BB, and/or anti-GITR, is administered systemically, and the bacteria are administered intratumorally. In some embodiments, the agonistic antibody, e.g., anti-OX40 antibody, anti-41BB, and/or anti-GITR, and bacteria are administered systemically. In some embodiments, one or more engineered bacteria described herein, which comprise gene sequence(s) encoding a metabolic converter, e.g., kynurenine consumers, adenosine consumers, arginine producers and/or ammonia consumers, and/or STING agonist producers, are administered sequentially, simultaneously, or subsequently to dosing with an anti-OX40 antibody. In some embodiments, one or more engineered bacteria described herein, which comprise gene sequence(s) encoding a metabolic converter, e.g., kynurenine consumers, adenosine consumers, arginine producers, ammonia consumers and/or STING agonist producers, are administered sequentially, simultaneously, or subsequently to dosing with an anti-41BB antibody. In some embodiments, genetically engineered bacteria expressing any one or more of the described circuits for the degradation of adenosine are administered sequentially, simultaneously, or subsequently to dosing with an anti-GITR antibody.

In some embodiments, genetically engineered bacteria expressing any one or more of the described circuits for the degradation of adenosine are administered sequentially, simultaneously, or subsequently to dosing with an agonistic immune stimulatory molecule, e.g., an agonistic antibody, e.g., anti-OX40 antibody, anti-41BB antibody, and/or anti-GITR antibody. In some embodiments, genetically engineered bacteria expressing any one or more of the described circuits for the degradation of adenosine are administered sequentially, simultaneously, or subsequently to dosing with anti-Ox40 antibody. In some embodiments, genetically engineered bacteria expressing any one or more of the described circuits for the degradation of adenosine are administered sequentially, simultaneously, or subsequently to dosing with anti-41BB antibody. In some embodiments, genetically engineered bacteria expressing any one or more of the described circuits for the degradation of adenosine are administered sequentially, simultaneously, or subsequently to dosing with anti-GITR antibody. In some embodiments, genetically engineered bacteria of expressing any one or more of the described circuits for the degradation of adenosine are administered sequentially, simultaneously, or subsequently to dosing with an anti-GITR antibody and anti-OX40 antibody and/or anti-41BB antibody.

In some embodiments, genetically engineered bacteria expressing any one or more of the described circuits for the consumption of kynurenine are administered sequentially, simultaneously, or subsequently to dosing with an agonistic immune stimulatory molecule, e.g., an agonistic antibody, e.g., anti-OX40 antibody, anti-41BB antibody, and/or anti-GITR antibody. In some embodiments, genetically engineered bacteria expressing any one or more of the described circuits for the consumption of kynurenine are administered sequentially, simultaneously, or subsequently to dosing with an anti-GITR antibody. In some embodiments, genetically engineered bacteria expressing any one or more of the described circuits for the consumption of kynurenine are administered sequentially, simultaneously, or subsequently to dosing with anti-OX40 antibody. In some embodiments, genetically engineered bacteria expressing any one or more of the described circuits for the consumption of kynurenine are administered sequentially, simultaneously, or subsequently to dosing with anti-41BB antibody. In some embodiments, genetically engineered bacteria of expressing any one or more of the described circuits for the consumption of kynurenine are administered sequentially, simultaneously, or subsequently to dosing with an anti-GITR antibody and anti-OX40 antibody and/or anti-41BB antibody.

In some embodiments, genetically engineered bacteria expressing any one or more of the described circuits for the production of arginine and/or consumption of ammonia are administered sequentially, simultaneously, or subsequently to dosing with an agonistic immune stimulatory molecule, e.g., an agonistic antibody, e.g., anti-OX40 antibody, anti-41BB antibody, and/or anti-GITR antibody. In some embodiments, genetically engineered bacteria expressing any one or more of the described circuits for the production of arginine and/or consumption of ammonia are administered sequentially, simultaneously, or subsequently to dosing with an anti-GITR antibody. In some embodiments, genetically engineered bacteria expressing any one or more of the described circuits for the production of arginine and/or consumption of ammonia are administered sequentially, simultaneously, or subsequently to dosing with anti-OX40 antibody. In some embodiments, genetically engineered bacteria expressing any one or more of the described circuits for the production of arginine and/or consumption of ammonia are administered sequentially, simultaneously, or subsequently to dosing with anti-OX40L antibody. In some embodiments, genetically engineered bacteria of expressing any one or more of the described circuits for the production of arginine and/or consumption of ammonia are administered sequentially, simultaneously, or subsequently to dosing with an anti-GITR antibody and anti-OX40 antibody.

In some embodiments, genetically engineered bacteria expressing any one or more of the described circuits for the production of one or more STING agonists are administered sequentially, simultaneously, or subsequently to dosing with an agonistic immune stimulatory molecule, e.g., an agonistic antibody, e.g., anti-OX40 antibody, anti-41BB antibody, and/or anti-GITR antibody. In some embodiments, genetically engineered bacteria expressing any one or more of the described circuits for the production of one or more STING agonists are administered sequentially, simultaneously, or subsequently to dosing with an anti-GITR antibody. In some embodiments, genetically engineered bacteria expressing any one or more of the described circuits for the production of one or more STING agonists are administered sequentially, simultaneously, or subsequently to dosing with anti-OX40 antibody. In some embodiments, genetically engineered bacteria expressing any one or more of the described circuits for the production of one or more STING agonists are administered sequentially, simultaneously, or subsequently to dosing with anti-41BB antibody. In some embodiments, genetically engineered bacteria of expressing any one or more of the described circuits for the production of one or more STING agonists are administered sequentially, simultaneously, or subsequently to dosing with an anti-GITR antibody and anti-OX40 antibody and/or anti-41BB antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria expressing any one or more of the described circuits for the production of one or more STING agonists and one or more metabolic conversion circuits are administered sequentially, simultaneously, or subsequently to dosing with a agonistic antibody or other immune stimulatory agonist, e.g., as described herein and known in the art, including but not limited to anti-OX40, anti-41BB, and anti-GITR. In some embodiments, one or more genetically engineered bacteria expressing any one or more of the described circuits for the production of one or more STING agonists and one or more metabolic conversion circuits are administered sequentially, simultaneously, or subsequently to dosing with an anti-GITR antibody. In some embodiments, one or more genetically engineered bacteria expressing any one or more of the described circuits for the production of one or more STING agonists and one or more metabolic conversion circuits are administered sequentially, simultaneously, or subsequently to dosing with anti-OX40 antibody. In some embodiments, one or more genetically engineered bacteria expressing any one or more of the described circuits for the production of one or more STING agonists and one or more metabolic conversion circuits are administered sequentially, simultaneously, or subsequently to dosing with anti-41BB antibody. In some embodiments, genetically engineered bacteria of expressing any one or more of the described circuits for the production of one or more STING agonists and one or more metabolic conversion circuits are administered sequentially, simultaneously, or subsequently to dosing with an anti-GITR antibody and anti-OX40 antibody and/or anti-41BB antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with a agonistic antibody or other immune stimulatory agonist, e.g., as described herein and known in the art, including but not limited to anti-OX40, anti-41BB, and anti-GITR. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with an anti-GITR antibody. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with anti-OX40 antibody. In some embodiments, one or more genetically engineered bacteria expressing any one or more of the described circuits for the production of one or more STING agonists and one or more kynureninase(s), e.g., from Pseudomonas fluorescens, are administered sequentially, simultaneously, or subsequently to dosing with anti-41BB antibody. In some embodiments, genetically engineered bacteria of expressing any one or more of the described circuits for the production of one or more STING agonists and one or more kynureninase(s), e.g., from Pseudomonas fluorescens, are administered sequentially, simultaneously, or subsequently to dosing with an anti-GITR antibody and anti-OX40 antibody and/or anti-41BB antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria expressing any one or more of the described circuits for the production of one or more STING agonists and one or more adenosine consuming enzymes described herein are administered sequentially, simultaneously, or subsequently to dosing with a agonistic antibody or other immune stimulatory agonist, e.g., as described herein and known in the art, including but not limited to anti-OX40, anti-41BB, and anti-GITR. In some embodiments, one or more genetically engineered bacteria expressing any one or more of the described circuits for the production of one or more STING agonists and one or more adenosine consuming enzymes described herein are administered sequentially, simultaneously, or subsequently to dosing with an anti-GITR antibody. In some embodiments, one or more genetically engineered bacteria expressing any one or more of the described circuits for the production of one or more STING agonists and one or more adenosine consuming enzymes described herein are administered sequentially, simultaneously, or subsequently to dosing with anti-OX40 antibody. In some embodiments, one or more genetically engineered bacteria expressing any one or more of the described circuits for the production of one or more STING agonists and one or more adenosine consuming enzymes described herein are administered sequentially, simultaneously, or subsequently to dosing with anti-41BB antibody. In some embodiments, genetically engineered bacteria of expressing any one or more of the described circuits for the production of one or more STING agonists and one or more adenosine consuming enzymes described herein are administered sequentially, simultaneously, or subsequently to dosing with an anti-GITR antibody and anti-OX40 antibody and/or anti-41BB antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria comprising any one or more of the described circuits for the production of one or more STING agonists and one or more arginine production and/or ammonia consuming circuits described herein are administered sequentially, simultaneously, or subsequently to dosing with a agonistic antibody or other immune stimulatory agonist, e.g., as described herein and known in the art, including but not limited to anti-OX40, anti-41BB, and anti-GITR. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with an anti-GITR antibody. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with anti-OX40 antibody. In some embodiments, genetically engineered bacteria comprising any one or more of the described circuits for the production of one or more STING agonists and one or more arginine production and/or ammonia consuming circuits described herein are administered sequentially, simultaneously, or subsequently to dosing with anti-41BB antibody. In some embodiments, genetically engineered bacteria of comprising any one or more of the described circuits for the production of one or more STING agonists and one or more arginine production and/or ammonia consuming circuits described herein are administered sequentially, simultaneously, or subsequently to dosing with an anti-GITR antibody and anti-OX40 antibody and/or anti-41BB antibody. In any of these embodiments, the genetically engineered bacteria may further comprise mutation(s) or deletion(s) in one or more essential genes. In any of these embodiments, the bacteria may comprise one or more auxotrophic modifications, e.g., mutations or deletions, dapA. In any of these embodiments, the bacteria may comprise one or more auxotrophic modifications, e.g., mutations or deletions, trpE. In any of these embodiments, the bacteria may comprise one or more auxotrophic modifications, e.g., mutations or deletions, thyA. In any of these embodiments, the bacteria may further comprise a phage modification, e.g., a mutation or deletion, in an endogenous prophage as described herein.

In some embodiments, one or more genetically engineered bacteria, e.g., adenosine consumer, kynurenine consumer, ammonia consumer, arginine producer, and/or STING agonist producer are able to improve anti-tumor activity (e.g., tumor proliferation, size, volume, weight) of the co-administered agonistic antibodies or other immune stimulatory agonist (e.g., anti-OX40, anti-41BB, and/or anti-GITR), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an agonistic antibody therapy alone under the same conditions or as compared to an unmodified bacteria of the same subtype under the same conditions. In some embodiments, one or more genetically engineered bacteria, e.g., adenosine consumer, kynurenine consumer, ammonia consumer, arginine producer, and/or STING agonist producer, are able to improve anti-tumor activity (e.g., tumor proliferation, size, volume, weight) of the co-administered agonistic antibodies or other immune stimulatory agonist (e.g., anti-OX40, anti-41BB, and/or anti-GITR), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, one or more genetically engineered bacteria, e.g., adenosine consumer, kynurenine consumer, ammonia consumer, arginine producer, and/or STING agonist producer, are able to improve anti-tumor activity (e.g., tumor proliferation, size, volume, weight) of the co-administered agonistic antibody or other immune stimulatory agonist (e.g., anti-OX40, anti-41BB, and/or anti-GITR), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to consume adenosine are able to improve anti-tumor activity (e.g., tumor proliferation, size, volume, weight) of the co-administered agonistic antibodies or other immune stimulatory agonist (e.g., OX40 and/or GITR), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an agonistic antibody therapy alone under the same conditions or as compared to an unmodified bacteria of the same subtype under the same conditions. In some embodiments, the bacteria genetically engineered to consume adenosine are able to improve anti-tumor activity (e.g., tumor proliferation, size, volume, weight) of the co-administered agonistic antibodies or other immune stimulatory agonist (e.g., anti-OX40, anti-41BB, and/or anti-GITR), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to consume adenosine are able to improve anti-tumor activity (e.g., tumor proliferation, size, volume, weight) of the co-administered agonistic antibody or other immune stimulatory agonist (e.g., anti-OX40, anti-41BB, and/or anti-GITR), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to consume kynurenine are able to improve anti-tumor activity (e.g., tumor proliferation, size, volume, weight) of the co-administered agonistic antibodies or other immune stimulatory agonist (e.g., anti-OX40, anti-41BB, and/or anti-GITR), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an agonistic antibody therapy alone under the same conditions or as compared to an unmodified bacteria of the same subtype under the same conditions. In some embodiments, the bacteria genetically engineered to consume kynurenine are able to improve anti-tumor activity (e.g., tumor proliferation, size, volume, weight) of the co-administered agonistic antibodies or other immune stimulatory agonist (e.g., anti-OX40, anti-41BB and/or anti-GITR), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to consume kynurenine are able to improve anti-tumor activity (e.g., tumor proliferation, size, volume, weight) of the co-administered agonistic antibody or other immune stimulatory agonist (e.g., anti-OX40, anti-41BB and/or anti-GITR), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to produce arginine and/or consume ammonia are able to improve anti-tumor activity (e.g., tumor proliferation, size, volume, weight) of the co-administered agonistic antibodies or other immune stimulatory agonist (e.g., anti-OX40, anti-41BB and/or anti-GITR), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an agonistic antibody therapy alone under the same conditions or as compared to an unmodified bacteria of the same subtype under the same conditions. In some embodiments, the bacteria genetically engineered to produce arginine and/or consume ammonia are able to improve anti-tumor activity (e.g., tumor proliferation, size, volume, weight) of the co-administered agonistic antibodies or other immune stimulatory agonist (e.g., anti-OX40, anti-41BB and/or anti-GITR), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to produce arginine and/or consume ammonia are able to improve anti-tumor activity (e.g., tumor proliferation, size, volume, weight) of the co-administered agonistic antibody or other immune stimulatory agonist (e.g., anti-OX40, anti-41BB and/or anti-GITR), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the bacteria genetically engineered to produce STING agonist(s) are able to improve anti-tumor activity (e.g., tumor proliferation, size, volume, weight) of the co-administered agonistic antibodies or other immune stimulatory agonist (e.g., anti-OX40, anti-41BB, and/or anti-GITR), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an agonistic antibody therapy alone under the same conditions or as compared to an unmodified bacteria of the same subtype under the same conditions. In some embodiments, the bacteria genetically engineered to produce STING agonist(s) are able to improve anti-tumor activity (e.g., tumor proliferation, size, volume, weight) of the co-administered agonistic antibodies or other immune stimulatory agonist (e.g., anti-OX40, anti-41BB and/or anti-GITR), e.g., 1.0-1.2-fold, 1.2-1.4-fold, 1.4-1.6-fold, 1.6-1.8-fold, 1.8-2-fold, or two-fold more or more as compared to a chemotherapy alone under the same conditions or as compared to an unmodified bacteria of the same subtype under the same conditions. In some embodiments, the bacteria genetically engineered to produce STING agonist(s) are able to improve anti-tumor activity (e.g., tumor proliferation, size, volume, weight) of the co-administered agonistic antibody or other immune stimulatory agonist (e.g., anti-OX40, anti-41BB and/or anti-GITR), e.g., about three-fold, four-fold, about three-fold, four-fold, five-fold, six-fold, seven-fold, eight-fold, nine-fold, ten-fold, fifteen-fold, twenty-fold, thirty-fold, forty-fold, fifty-fold, hundred-fold, five hundred-fold, or one-thousand-fold more or more as compared to a chemotherapy alone under the same conditions or as compared to an unmodified bacteria of the same subtype under the same conditions.

In some embodiments, the genetically engineered microorganisms may be administered as part of a regimen, which includes other treatment modalities or combinations of other modalities. Non-limiting examples of these modalities or agents are conventional therapies (e.g., radiotherapy, chemotherapy), other immunotherapies, stem cell therapies, and targeted therapies, (e.g., BRAF or vascular endothelial growth factor inhibitors; antibodies or compounds), bacteria described herein, and oncolytic viruses. Therapies also include related to antibody-immune engagement, including Fc-mediated ADCC therapies, therapies using bispecific soluble scFvs linking cytotoxic T cells to tumor cells (e.g., BiTE), and soluble TCRs with effector functions. Immunotherapies include vaccines (e.g., viral antigen, tumor associated antigen, neoantigen, or combinations thereof), checkpoint inhibitors, cytokine therapies, adoptive cellular therapy (ACT). ACT includes but is not limited to, tumor infiltrating lymphocyte (TIL) therapies, native or engineered TCR or CAR-T therapies, natural killer cell therapies, and dendritic cell vaccines or other vaccines of other antigen presenting cells. Targeted therapies include antibodies and chemical compounds, and include for example antiangiogenic strategies and BRAF inhibition.

The immunostimulatory activity of bacterial DNA is mimicked by synthetic oligodeoxynucleotides (ODNs) expressing unmethylated CpG motifs. Bode et al., Expert Rev Vaccines. 2011 April; 10(4): 499-511. CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the genetically engineered bacteria of the invention.

In one embodiment, the genetically engineered microorganisms are administered in combination with tumor cell lysates.

The dosage of the pharmaceutical composition and the frequency of administration may be selected based on the severity of the symptoms and the progression of the cancer. The appropriate therapeutically effective dose and the frequency of administration can be selected by a treating clinician.

### Treatment In Vivo

The modified microorgansims may be evaluated in vivo, e.g., in an animal model. Any suitable animal model of a disease or condition associated with cancer may be used, e.g., a tumor syngeneic or xenograft mouse models (see, e.g., Yu et al., 2015). The genetically engineered bacteria may be administered to the animal systemically or locally, e.g., via oral administration (gavage), intravenous, or subcutaneous injection or via intratumoral injection, and treatment efficacy determined, e.g., by measuring tumor volume.

Non-limiting examples of animal models include mouse models, as described in Dang et al., 2001, Heap et al., 2014 and Danino et al., 2015).

Pre-clinical mouse models determine which immunotherapies and combination immunotherapies will generate the optimal therapeutic index (maximal anti-tumor efficacy and minimal immune related adverse events (irAEs)) in different cancers.

Implantation of cultured cells derived from various human cancer cell types or a patient's tumor mass into mouse tissue sites has been widely used for generations of cancer mouse models (xenograft modeling). In xenograft modeling, human tumors or cell lines are implanted either subcutaneously or orthotopically into immune-compromised host animals (e.g., nude or SCID mice) to avoid graft rejection. Because the original human tumor microenvironment is not recapitulated in such models, the activity of anti-cancer agents that target immune modulators may not be accurately measured in these models, making mouse models with an intact immune system more desirable.

Accordingly, implantation of murine cancer cells in a syngeneic immunocompetent host (allograft) are used to generate mouse models with tumor tissues derived from the same genetic background as a given mouse strain. In syngeneic models, the host immune system is normal, which may more closely represent the real life situation of the tumor's micro-environment. The tumor cells or cancer cell lines are implanted either subcutaneously or orthotopically into the syngeneic immunocompetent host animal (e.g., mouse). Representative murine tumor cell lines, which can be used in syngeneic mouse models for immune checkpoint benchmarking include, but are not limited to the cell lines listed in International Patent Application PCT/US2017/013072, filed Jan. 11, 2017, published as WO2017/123675, the contents of which is herein incorporated by reference in its entirety.

For tumors derived from certain cell lines, ovalbumin can be added to further stimulate the immune response, thereby increasing the response baseline level. Examples of mouse strains that can be used in syngeneic mouse models, depending on the cell line include C57BL/6, FVB/N, Balb/c, C3H, HeJ, C3H/HeJ, NOD/ShiLT, NJ, 129S1/Sv1 mJ, NOD. Additionally, several further genetically engineered mouse strains have been reported to mimic human tumorigenesis at both molecular and histologic levels. These genetically engineered mouse models also provide excellent tools to the field and additionally, the cancer cell lines derived from the invasive tumors developed in these models are also good resources for cell lines for syngeneic tumor models Examples of genetically engineered strains are provided in in International Patent Application PCT/US2017/013072, filed Jan. 11, 2017, published as WO2017/123675, the contents of which is herein incorporated by reference in its entirety.

Often potential therapeutic molecules which interact with human immune modulators and stimulate human immune system and do not detect their murine counterparts and vice versa. In studying therapeutic molecules, it is necessary to take this in consideration. More recently, “humanized” mouse models have been developed, in which immunodeficient mice are reconstituted with a human immune system, and which have helped overcome issues relating to the differences between the mouse and human immune systems, allowing the in vivo study of human immunity. Severely immunodeficient mice which combine the IL2receptor null and the severe combined immune deficiency mutation (scid) (NOD-scid IL2Rgnull mice) lack mature T cells, B cells, or functional NK cells, and are deficient in cytokine signaling. These mice can be engrafted with human hematopoietic stem cells and peripheral-blood mononuclear cells. CD34+ hematopoietic stem cells (hu-CD34) are injected into the immune deficient mice, resulting in multi-lineage engraftment of human immune cell populations including very good T cell maturation and function for long-term studies. This model has a research span of 12 months with a functional human immune system displaying T-cell dependent inflammatory responses with no donor cell immune reactivity towards the host. Patient derived xenografts can readily be implanted in these models and the effects of immune modulatory agents studied in an in vivo setting more reflective of the human tumor microenvironment (both immune and non-immune cell-based) (Baia et al., 2015). Human cell lines of interest for use in the humanized mouse models include but are not limited to HCT-116 and HT-29 colon cancer cell lines.

A rat F98 glioma model and the utility of spontaneous canine tumors, as described in Roberts et al 2014, the contents of each of which are herein incorporated by reference in their entireties. Locally invasive tumors generated by implantation of F98 rat glioma cells engineered to express luciferase were intratumorally injected with C. novyi-NT spores, resulting in germination and a rapid fall in luciferase activity. C. novyi-NT germination was demonstrated by the appearance of vegetative forms of the bacterium. In these studies, C. novyi-NT precisely honed to the tumor sparing neighboring cells.

Canine soft tissue sarcomas for example are common in many breeds and have clinical, histopathological, and genetically features similar to those in humans (Roberts et al, 2014; Staedtke et al., 2015), in particular, in terms of genetic alterations and spectrum of mutations. Roberts et al. conducted a study in dogs, in which C. novyi-NT spores were intratumorally injected (1×10⁸ C. novyi-NT spores) into spontaneously occurring solid tumors in one to 4 treatment cycles and followed for 90 days. A potent inflammatory response was observed, indicating that the intratumoral injections mounted an innate immune response.

In some embodiments, the genetically engineered microorganisms of the invention are administered systemically, e.g., orally, subcutaneously, intravenously or intratumorally into any of the models described herein to assess anti-tumor efficacy and any treatment related adverse side effects.

### Sequences

In some embodiments, certain precursor sequences are replaced with one or more bacterial sequences, including but not limited to bacterial secretion signal sequences. In some embodiments, the polynucleotide sequence encoding the cytokines are codon-optimized for bacterial expression. In some embodiments, certain precursor sequences are replaced with one or more mammalian sequences, including but not limited to mammalian secretion signal sequences. In some embodiments, the polynucleotide sequence encoding the cytokines are codon-optimized for mammalian expression.

In some embodiments, the genetically engineered bacteria comprise gene sequences encoding immune modulatory cytokines. In some embodiments, the genetically engineered bacteria comprise one or more gene sequences for the expression of hIL-12. In some embodiments, genetically engineered bacteria comprise one or more gene sequences that encode a polypeptide of SEQ ID NO: 1053. In some embodiments, genetically engineered bacteria comprise one or more gene sequences that encode a polypeptide of SEQ ID NO: 1054.

In some embodiments, the genetically engineered bacteria comprise one or more gene sequences for the expression of hIL-15. In some embodiments, genetically engineered bacteria comprise one or more gene sequences that encode a polypeptide of SEQ ID NO: 1057.

In some embodiments, the genetically engineered bacteria comprise one or more gene sequences for the expression of GMCSF. In some embodiments, genetically engineered bacteria comprise one or more gene sequences that encode a polypeptide of SEQ ID NO: 1058.

In some embodiments, the genetically engineered bacteria comprise one or more gene sequences for the expression of TNFα, e.g., the extracellular portion. In some embodiments, genetically engineered bacteria comprise one or more gene sequences that encode a polypeptide of SEQ ID NO: 1059.

In some embodiments, the genetically engineered bacteria comprise one or more gene sequences for the expression of IFN-gamma. In some embodiments, genetically engineered bacteria comprise one or more gene sequences that encode a polypeptide of SEQ ID NO: 1060.

In some embodiments, the genetically engineered bacteria comprise one or more gene sequences for the expression of CXCL10. In some embodiments, genetically engineered bacteria comprise one or more gene sequences that encode a polypeptide of SEQ ID NO: 1061.

In some embodiments, the genetically engineered bacteria comprise one or more gene sequences for the expression of CXCL9. In some embodiments, genetically engineered bacteria comprise one or more gene sequences that encode a polypeptide of SEQ ID NO: 1062.

In some embodiments, genetically engineered bacteria comprise a nucleic acid sequence that encodes a polypeptide that is at least about 80%, at least about 85%, at least about 90%, at least about 95%, or at least about 99% homologous to SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061 SEQ, and ID NO: 1062. In some embodiments, genetically engineered bacteria comprise a nucleic acid sequence that encodes a polypeptide that comprise a sequence selected from SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061 SEQ, and ID NO: 1062. In some embodiments, genetically engineered bacteria comprise a nucleic acid sequence that encodes a polypeptide that consists of a sequence selected from SEQ ID NO: 1053, SEQ ID NO: 1054, SEQ ID NO: 1055, SEQ ID NO: 1056, SEQ ID NO: 1057, SEQ ID NO: 1058, SEQ ID NO: 1059, SEQ ID NO: 1060, SEQ ID NO: 1061 SEQ, and ID NO: 1062.

In some embodiments, the genetically engineered bacteria comprise a gene sequence that but for the redundancy of the genetic code encodes the same polypeptide as SEQ ID NO: 1063. In some embodiments, the genetically engineered bacteria comprise SEQ ID NO: 1063. In some embodiments, the genetically engineered bacteria comprise a gene sequence that but for the redundancy of the genetic code encodes the same polypeptide as SEQ ID NO: 1064. In some embodiments, the genetically engineered bacteria comprise a gene sequence SEQ ID NO: 1064. In some embodiments, the genetically engineered bacteria comprise a gene sequence that but for the redundancy of the genetic code encodes the same polypeptide as SEQ ID NO: 1067 In some embodiments, the genetically engineered bacteria comprise SEQ ID NO: 1067. In some embodiments, the genetically engineered bacteria comprise a gene sequence that but for the redundancy of the genetic code encodes the same polypeptide as SEQ ID NO: 1068 In some embodiments, the genetically engineered bacteria comprise SEQ ID NO: 1068. In some embodiments, the genetically engineered bacteria comprise a gene sequence that but for the redundancy of the genetic code encodes the same polypeptide as SEQ ID NO: 1069 In some embodiments, the genetically engineered bacteria comprise SEQ ID NO: 1069. In some embodiments, the genetically engineered bacteria comprise a gene sequence In some embodiments, the genetically engineered bacteria comprise a gene sequence that but for the redundancy of the genetic code encodes the same polypeptide as SEQ ID NO: 1070. In some embodiments, the genetically engineered bacteria comprise SEQ ID NO: 1070 In some embodiments, the genetically engineered bacteria comprise a gene sequence that but for the redundancy of the genetic code encodes the same polypeptide as SEQ ID NO: 1071 In some embodiments, the genetically engineered bacteria comprise SEQ ID NO: 1071. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 1063, SEQ ID NO: 1064, SEQ ID NO: 1067, SEQ ID NO: 1068, SEQ ID NO: 1069, SEQ ID NO: 1070, and/or SEQ ID NO: 1071. In some embodiments, genetically engineered bacteria comprise a nucleic acid sequence comprising a sequence selected from SEQ ID NO: 1063, SEQ ID NO: 1064, SEQ ID NO: 1067, SEQ ID NO: 1068, SEQ ID NO: 1069, SEQ ID NO: 1070, and/or SEQ ID NO: 1071. In some embodiments, genetically engineered bacteria comprise a nucleic acid sequence consisting of a sequence selected from SEQ ID NO: 1063, SEQ ID NO: 1064, SEQ ID NO: 1067, SEQ ID NO: 1068, SEQ ID NO: 1069, SEQ ID NO: 1070, and/or SEQ ID NO: 1071.

In some embodiments, genetically engineered bacteria comprise a nucleic acid sequence that is at least about 80%, at least about 85%, at least about 90%, at least about 95%, or at least about 99% homologous to the DNA sequence of SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, and/or SEQ ID NO: 913. In some embodiments, genetically engineered bacteria comprise a nucleic acid sequence comprising a the DNA sequence selected from SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, and/or SEQ ID NO: 913. In some embodiments, genetically engineered bacteria comprise a nucleic acid sequence consisting of DNA sequence selected from SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, and/or SEQ ID NO: 913.

In some embodiments, genetically engineered bacteria comprise a nucleic acid sequence that is at least about 80%, at least about 85%, at least about 90%, at least about 95%, or at least about 99% homologous to the DNA sequence of SEQ ID NO: 914, SEQ ID NO: 915, SEQ ID NO: 916, SEQ ID NO: 917, SEQ ID NO: 918, and SEQ ID NO: 919. In some embodiments, genetically engineered bacteria comprise a nucleic acid sequence comprising a DNA sequence selected from SEQ ID NO: 914, SEQ ID NO: 915, SEQ ID NO: 916, SEQ ID NO: 917, SEQ ID NO: 918, and SEQ ID NO: 919. In some embodiments, genetically engineered bacteria comprise a nucleic acid sequence consisting of a DNA sequence selected from SEQ ID NO: 914, SEQ ID NO: 915, SEQ ID NO: 916, SEQ ID NO: 917, SEQ ID NO: 918, and SEQ ID NO: 919.

In one embodiment, genetically engineered bacteria comprise a gene sequence encoding a human IL-12a construct with a N terminal OmpF secretion tag, e.g., SEQ ID NO: 920. In one embodiment, genetically engineered bacteria comprise a gene sequence encoding a human IL-12a construct with a N terminal PhoA secretion tag, e.g., SEQ ID NO: 921. In one embodiment, genetically engineered bacteria comprise a gene sequence encoding a human IL-12a construct with a N terminal TorA secretion tag, e.g., SEQ ID NO: 922. In one embodiment, genetically engineered bacteria comprise a gene sequence encoding a human IL-12b construct with a N terminal OmpF secretion tag, e.g., SEQ ID NO: 923. In one embodiment, genetically engineered bacteria comprise a gene sequence encoding a human IL-12b construct with a N terminal PhoA secretion tag, e.g., SEQ ID NO: 924. In one embodiment, genetically engineered bacteria comprise a gene sequence encoding a human IL-12 construct with a N terminal TorA secretion tag, e.g., SEQ ID NO: 925. In one embodiment, genetically engineered bacteria comprise a gene sequence encoding a human GMCSF construct with a N terminal OmpF secretion tag, e.g., SEQ ID NO: 932. In one embodiment, genetically engineered bacteria comprise a gene sequence encoding a human GMCSF construct with a N terminal PhoA secretion tag, e.g., SEQ ID NO: 933. In one embodiment, genetically engineered bacteria comprise a gene sequence encoding a human GMCSF construct with a N terminal TorA secretion tag, e.g., SEQ ID NO: 934.

In one embodiment, genetically engineered bacteria comprise a gene sequence encoding a human IL-15 construct with a N terminal OmpF secretion tag, e.g., SEQ ID NO: 935. In one embodiment, genetically engineered bacteria comprise a gene sequence encoding a human IL-15 construct with a N terminal PhoA secretion tag, e.g., SEQ ID NO: 936. In one embodiment, genetically engineered bacteria comprise a gene sequence encoding a human IL-15 construct with a N terminal TorA secretion tag, e.g., SEQ ID NO: 937. In one embodiment, genetically engineered bacteria comprise a gene sequence encoding a human TNFα construct with a N terminal OmpF secretion tag, e.g., SEQ ID NO: 938. In one embodiment, genetically engineered bacteria comprise a gene sequence encoding a human TNFα construct with a N terminal PhoA secretion tag, e.g., SEQ ID NO: 939. In one embodiment, genetically engineered bacteria comprise a gene sequence encoding a human TNFα construct with a N terminal TorA secretion tag, e.g., SEQ ID NO: 940. In one embodiment, genetically engineered bacteria comprise a gene sequence encoding a human IFNg construct with a N terminal OmpF secretion tag, e.g., SEQ ID NO: 941. In one embodiment, genetically engineered bacteria comprise a gene sequence encoding a human IFNg construct with a N terminal PhoA secretion tag, e.g., SEQ ID NO: 942. In one embodiment, genetically engineered bacteria comprise a gene sequence encoding a human IFNgamma construct with a N terminal TorA secretion tag, e.g., SEQ ID NO: 943. In one embodiment, genetically engineered bacteria comprise a gene sequence encoding a human CXCL9 construct with a N terminal OmpF secretion, e.g., SEQ ID NO: 1075. In one embodiment, genetically engineered bacteria comprise a gene sequence encoding a human CXCL9 construct with a N terminal PhoA secretion tag, e.g., SEQ ID NO: 1076. In one embodiment, genetically engineered bacteria comprise a gene sequence encoding a human CXCL9 construct with a N terminal TorA secretion tag, e.g., SEQ ID NO: 1077.

In some embodiments, genetically engineered bacteria comprise a gene sequence, which encodes a polypeptide that is at least about 80%, at least about 85%, at least about 90%, at least about 95%, or at least about 99% homologous to a polypeptide sequence selected from SEQ ID NOs: 920-943 or 1072-1078, or a functional fragment or variant thereof. In some embodiments, genetically engineered bacteria comprise a gene sequence, which encodes a polypeptide that is at least about 80%, at least about 85%, at least about 90%, at least about 95%, or at least about 99% homologous to a polypeptide sequence selected from SEQ ID NOs: 920-943 or 1072-1078. In some embodiments, genetically engineered bacteria comprise a gene sequence, which encodes a polypeptide comprising a sequence selected from SEQ ID NOs: 920-943 or 1072-1078. In some embodiments, genetically engineered bacteria comprise a gene sequence, which encodes a polypeptide consisting of a sequence selected from SEQ ID NOs: 920-943 or 1072-1078.

In some embodiments, genetically engineered bacteria comprise one or more nucleic acid sequences selected from SEQ ID NOs: 953-960 and SEQ ID NOs: 1081-1084. In some embodiments, genetically engineered bacteria comprise a nucleic acid sequence that is at least about 80%, at least about 85%, at least about 90%, at least about 95%, or at least about 99% homologous to one or more DNA sequences selected from SEQ ID NOs: 953-960 and SEQ ID NOs: 1081-1084.

In one embodiment, genetically engineered bacteria comprise a gene sequence comprising a construct comprising both human IL-12a and human IL-12b. In one embodiment, genetically engineered bacteria comprise a gene sequence comprising the phoA-hIL12b-phoA-hIL12a portion of SEQ ID NO: 965 or the phoA-mIL12b-phoA-mIL12a portion of SEQ ID NO: 966.

In one embodiment, genetically engineered bacteria comprise a gene sequence comprising a construct comprising phoA-IL15. In one embodiment, genetically engineered bacteria comprise a gene sequence comprising the phoA-IL15 portion of SEQ ID NO: 967

In one embodiment, genetically engineered bacteria comprise a gene sequence comprising a construct comprising phoA-GMCSF. In one embodiment, genetically engineered bacteria comprise a gene sequence comprising the phoA-GMCSF portion of SEQ ID NO: 968.

In one embodiment, genetically engineered bacteria comprise a gene sequence comprising a construct comprising phoA-TNFα. In one embodiment, genetically engineered bacteria comprise a gene sequence comprising the phoA-TNFα portion of SEQ ID NO: 969.

In one embodiment, genetically engineered bacteria comprise a gene sequence comprising a construct comprising phoA-IFNgamma. In one embodiment, genetically engineered bacteria comprise a gene sequence comprising the phoA-IFNgamma portion of SEQ ID NO: 970.

In some embodiments, genetically engineered bacteria comprise a nucleic acid sequence that is at least about 80%, at least about 85%, at least about 90%, at least about 95%, or at least about 99% homologous a DNA sequence selected from SEQ ID NOs: 965, SEQ ID NO: 966, SEQ ID NO: 967, SEQ ID NO: 968, SEQ ID NO: 969, SEQ ID NO: 970, excluding the non-coding regions.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

The disclosure provides herein a sequence having at least about 80%, at least about 85%, at least about 90%, at least about 95%, or at least about 99% homologous to the sequence any of the SEQ ID NOs described in the Examples, below.

### Example 1. Immune Modulators

Exemplary nucleic acid sequences for use in constructing single-chain anti-CTLA-4 antibodies are described in International Patent Application PCT/US2017/013072, filed Jan. 11, 2017, published as WO2017/123675, the contents of which is herein incorporated by reference in its entirety, for example in Example 1. In some embodiments, genetically engineered bacteria comprise a nucleic acid sequence that is at least about 80%, at least about 85%, at least about 90%, at least about 95%, or at least about 99% homologous to the DNA sequence or comprises a DNA sequence that encodes a polypeptide that is at least about 80%, at least about 85%, at least about 90%, at least about 95%, or at least about 99% homologous to SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, and/or SEQ ID NO: 776. In another embodiment, the genetically engineered bacteria comprise a gene sequence encoding a polypeptide comprising a sequence selected from SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, and/or SEQ ID NO: 776. In yet another embodiment, the polypeptide expressed by the genetically engineered bacteria consists of a sequence selected from SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, and/or SEQ ID NO: 776.

In some embodiments, genetically engineered bacteria comprise a nucleic acid sequence that is at least about 80%, at least about 85%, at least about 90%, at least about 95%, or at least about 99% homologous to the DNA sequence or comprises a DNA sequence that encodes a polypeptide that is at least about 80%, at least about 85%, at least about 90%, at least about 95%, or at least about 99% homologous to SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, and/or SEQ ID NO: 788. In another embodiment, the gene sequence comprises a sequence selected from SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, and/or SEQ ID NO: 788. In another embodiment, the gene sequences consists of a sequence selected from SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, and/or SEQ ID NO: 788.

### Example 2. Tumor Pharmacokinetics for E. coli Nissle

Tumor pharmacokinetics were assayed and determined as described in are described in International Patent Application PCT/US2017/013072, filed Jan. 11, 2017, published as WO2017/123675, the contents of which is herein incorporated by reference in its entirety, e.g., Example 58-61. Tumor pharmacokinetics of Nissle (1e7 and 1e8 cells/dose) were determined using a CT26 tumor model over 7 days. Bacterial counts in the tumor tissue were similar at both doses. No bacteria were detected in blood at any of the time points.

Tumor pharmacokinetics of streptomycin resistant Nissle and a Nissle DOM mutant (Nissle ΔPAL::CmR) were compared in a CT26 tumor model. Bacterial counts in the tumor tissue were similar in both strains. No bacteria were detected in the blood. These results indicate that both the wild type and the DOM mutant Nissle can survive in the tumor environment.

Cytokine response in vivo to intratumoral administration of streptomycin resistant Nissle was assessed using a CT26 tumor model at either 1e6 (Group1) or 1e7 cells/dose (Group 2). Levels measured in serum and in the tumor over the time course post SYN94 intratumoral administration in the mouse CT-24 model at the indicated doses. Results indicate that a cytokine response is elicited in the tumor at the higher dose but not in the serum. The lower dose does not elicit a substantial cytokine response.

Tumoral PK, levels of bacteria in various tissues and cytokine levels in these tissues were assessed post IT dosing (1e7 cells/dose) at 48 hours. As seen in Internationals Patent Application PCT/US2017/013072, incorporated herein by reference, bacteria were predominantly present in the tumor and absent in other tissues tested. TNFα levels measured were similar in all serum, tumor and liver between SYN94, Saline treated and naïve groups. TNFα levels are negligible relative to TNFα levels measured at 1.5 hours when Nissle is administered at 1e8 via IV. However, even with IV administration, TNFα levels drop off to undetectable levels at 4 hours Similar low levels of TNFα are detected at a 1e6 IV dose of SYN94.

### Example 3A. LC-MS/MS Quantification of Metabolites

Quantification of adenosine, kynurenine, tryptophan and arginine in bacterial supernatants and in tumor tissues was performed by LC-MS/MS as described in International Patent Application PCT/US2017/013072, filed Jan. 11, 2017, published as WO2017/123675, the contents of which is herein incorporated by reference in its entirety.

### Example 3B. Engineering Bacterial Strains Using Chromosomal Insertions

Methods for the integration of constructs into the E. coli Nissle genome are described in Internationals Patent Application PCT/US2017/013072, incorporated herein by reference.

### Example 4. Generation of Adenosine Degrading Strains

A schematic representation of the 3 operons in the adenosine degradation pathway is shown in FIG. 47 and FIG. 48. To generate Adenosine consuming strains, each one of the operons (or single gene in the case of nupC) were cloned into a KIKO vector under the control of the p_{fnrS} promoter. Knock-in PCR products were made from the KIKO vectors and allelic exchange was performed to integrate these operons into E. coli genome. Allelic exchange was facilitated through use of the lambda red recombinase system as described herein. Multiple strain combinations were generated and Table 13. summarizes the strains generated and compared in adenosine degradation assays. Table 14. summarizes the integration sites that were used for each of the constructs.

TABLE 13
Adenosine consuming strains

| Strain: | Genotype |
|---|---|
| SYN01 | WT |
| SYN1565 | P_{fnrS}-nupC |
| SYN1584 | P_{fnrS}-nupC; P_{fnrS}-xdhABC |
| SYN1655 | P_{fnrS}-nupC; P_{fnrS}-add-xapA-deoD |
| SYN1656 | P_{fnrS}-nupC; P_{fnrS}-xdhABC; P_{fnrS}-add-xapA-deoD |

TABLE 14
Integration sites (can also see strain table)

| Construct | Chromosomal Integration Site |
|---|---|
| P_{fnrS}-nupC | integrated into HA1/2 (ugaI/rsmI) region |
| P_{fnrS}-xdhABC | integrated into HA9/10 (exo/cea) region |
| P_{fnrS}-add-xapA-deoD | integrated into malE/K region |

### Example 5. In Vitro Adenosine Degradation Measurements

### In Vitro Adenosine Consumption

Glucose is the preferred carbon source of E. coli. However, E. coli can also use adenosine as a sole source of carbon in the absence of glucose. To assess the ability of the newly generated strains to degrade adenosine, and if able to do so even in the presence of the preferred carbon source, glucose.

To accomplish this, overnight cultures of each strain including a wild type control were grown in LB at 37 C, shaking at 250 rpm. Cultures were back diluted 1:100 (10 mL in 125 mL baffled flask) and grown for 1.5 hours to early log phase. Once cultures reached early log, cultures were moved into a Coy anaerobic chamber supplying an anaerobic atmosphere (85% N₂, 10% CO₂, 5% H₂). Cultures were incubated anaerobically for 4 hours to allow for induction of the engineered adenosine degradation pathway gene(s).

Cultures were removed from the anaerobic chamber and tested for adenosine degradation activity. To accomplish this, ˜1e8 activated bacterial cells were spun down in 1.5 mL microcentrifuge tubes and resuspended in adenosine assay buffer (1× M9 minimal media containing 10 mM adenosine that either contained no glucose or 0.5% glucose (see slide)). Tubes were incubated statically at 37 degrees Celsius for 5h, and supernatant samples were removed every hour for 5h. Supernatant samples were analyzed via LC-MS for determination of adenosine concentration.

Results are show in FIG. 3. and indicate that all engineered strains were able to degrade adenosine (determined by its absence in the supernatant samples) at a rate higher than that of the wild type control strain. All strains were able to degrade adenosine regardless whether E. coli's preferred carbon source, glucose, was present.

### In Vitro Activity Under Substrate Limited Conditions

In the previous study, substrate was not limiting, i.e., strains were able to function at Vₘₐₓ. Such substrate concentrations were far in excess of concentrations expected in vivo. Next, adenosine degradation ability of the engineered bacteria was assessed at more limiting substrate concentrations (more consistent with adenosine concentrations in a tumor in vivo), and at lower doses (more consistent with doses which can be administered IV or IT in a mouse without causing sepsis).

Overnight cultures of each strain were grown in LB at 37 C, shaking at 250 rpm. Cultures were back diluted 1:100 (10 mL in 125 mL baffled flask) and grown for 1.5 hours to early log phase. Once cultures reached early log, they were moved into a Coy anaerobic chamber supplying an anaerobic atmosphere (85% N₂, 10% CO₂, 5% H₂). Cultures were incubated anaerobically for 4 hours to allow for induction of the engineered adenosine degradation pathway gene(s).

Activated cells were quantitated on a cellometer and diluted in PBS to 5e8 cfu/mL. 10 uL of this suspension (comprising 5e6 bacteria) were resuspended in 1 mL of adenosine assay buffer comprised of M9 minimal media, 0.5% glucose, and 100 uM adenosine. Cells were incubated statically at 37 C. Supernatant samples were removed every hour for 5 hours to determine rates of adenosine degradation. Supernatant samples were analyzed via LC-MS for determination of adenosine concentration. Rates of degradation reported are the maximal linear rates between 0 to 5 hours of sampling (this may not include the later time points as rates may not be linear at extremely low substrate (adenosine) degradation).

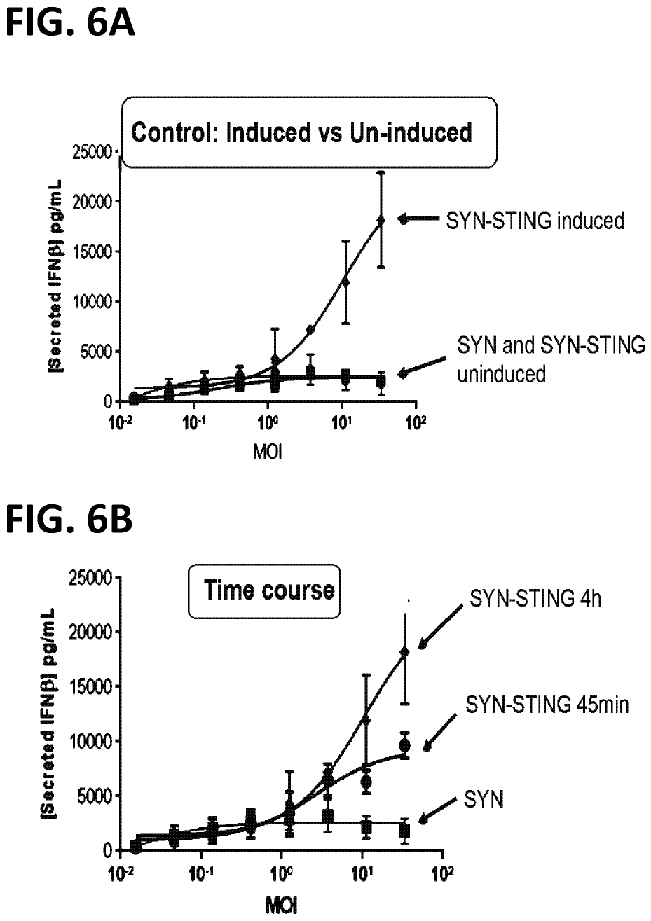

Results are shown in FIGS. 5 and 6 and indicate that all engineered strains were able to degrade adenosine (determined by its absence in the supernatant samples) at a rate higher than that of control strain SYN01. SYN1656, the most highly engineered strain containing all three integrations comprising the adenosine degradation pathway, was able to degrade adenosine at the highest rate and to take adenosine levels to undetectable levels by 3 hours.

The linear rate is shown in Table 15.

TABLE 15
Linear Adenosine Degradation Rates

| | Linear Rate (umol/hr/10⁹ cells) |
|---|---|
| SYN001 | 1.95 |
| SYN1552 | 5.90 |
| SYN1584 | 6.39 |
| SYN1655 | 5.65 |
| SYN1656 | 6.88 |

### Example 6. Effect of Adenosine Consuming Strains In Vivo

The effects of an adenosine consuming strain SYN1656 (comprising P_{fnrS}-nupC; P_{fnrS}-xdhABC; P_{fnrS}-add-xapA-deoD) in vivo was assessed, alone and in combination with anti-PD1.

CT26 cells obtained from ATCC were cultured according to guidelines provided. Approximately ˜1e6cells/mouse in PBS were implanted subcutaneously into the right flank of each animal (BalbC/J (female, 8 weeks)), and tumor growth was monitored for approximately 10 days. When the tumors reached about ˜100-150 mm3, animals were randomized into groups for dosing.

To prepare the cells, streptomycin resistant Nissle (SYN094) was grown in LB broth until reaching an absorbance at 600 nm (A600 nm) of 0.4 (corresponding to 2×108 colony-forming units (CFU)/mL) and washed twice in PBS. The suspension was diluted in PBS or saline so that 100 microL can be injected at the appropriate doses intratumorally into tumor-bearing mice. To prepare the SYN1656, cells were diluted 1:100 in LB (2 L), grown for 1.5 h aerobically, then shifted to the anaerobe chamber for 4 hours. Prior to administration, cells were concentrated 200× and frozen (15% glycerol, 2 g/L glucose, in PBS).

Approximately 10 days after CT 26 implantation, on day 1, bacteria were suspended in 0.1 ml of PBS and mice were weighed, measured, and randomized into treatment groups as follows: Group 1 saline injection (100 ul) (n=14); Group 2 SYN94 IT 10e7 (n=14); Group 3—SYN1656 IT 10e7 (n=14);

Group 4—SYN1656 IT 10e7 plus aPD-1 (BioXcell), 10 mg/kg, i.p. (n=14); Group 5—aPD-1 (BioXcell), 10 mg/kg, i.p (n=9).

On day 1 and day 4, animals were dosed according to their grouping either with saline or with the strains intratumorally (IT) alone or in combination with anti-PD1 (I.P). Plasma and were collected for further analysis. FIG. 49 shows the tumor volume of the mice from day 1, 4, and 7. Results show that the tumor volume is decreased in all three treatment groups (SYN1656, anti-PD1, and SYN1656 plus anti-PD1) as compared to the saline treated controls at 7 days; tumor size is smallest in the SYN1656 and anti-PD1 treated group, followed by SYN1656 alone and anti-PD1 alone, indicating that there may be a synergistic effect between the two treatments, and suggesting anti-tumor activity of adenosine-consuming strain as single agent and in combination with aPD-1. Tumor volume was significantly lower in the animals treated with SYN1656 and anti-PD1 than with saline alone (p=0.01). Tumor volume of animals treated with SYN1656 and anti-PD1 was also significantly lower than animals treated with anti-PD1 alone.

In other studies, this study is extended to include dosing and analysis at days 10, 15, and 18, until animals reach a tumor size of approximately 2000 mm³.

### Example 7. Adaptive Laboratory Evolution (ALE)

First, strains were generated, which comprise the trpE knock out and integrated constructs for the expression of Pseudomonas fluorescens KYNase driven by a constitutive promoter, as described in International Patent Application PCT/US2017/013072, filed on Jan. 11, 2017, published as WO2017/123675; and International Patent Application PCT/US2018/012698, filed on Jan. 5, 2018, the contents of which are incorporated by reference in their entirety. KYNase constructs were integrated at the HA3/4 site, and two different promoters were used; the promoter of the endogenous 1pp gene was used in parental strain SYN2027 (HA3/4::Plpp-pKYNase KanR TrpE::CmR) and the synthetic pSynJ23119 was used in parental strain SYN2028 (HA3/4::PSynJ23119-pKYNase KanR TrpE::CmR). These strains were generated so that a strain would be evolved, which would comprise a chromosomally integrated version of Pseudomonas fluorescens KYNase. These strains were validated in the checkerboard assay (e.g., as described in PCT/US2017/013072) to have similar ALE parameters to their plasmid-based Ptet counterpart. Lower limit of kynurenine (KYN) and ToxTrp concentration for use in the ALE experiment were established using the checkerboard assay described in PCT/US2017/013072, and lower limit concentrations corresponded to those observed for the strains expressing tet inducible KYNase from a medium copy plasmid.

Mutants derived from parental strains SYN2027 and SYN2028 were evolved by passaging in lowering concentrations of KYN and three different ToxTrp concentrations as follows.

The ALE parental strains were cultured on plates with M9 minimal media supplemented with glucose and L-kynurenine (M9+KYN). A single colony from each parent was selected, resuspended in 20 uL of sterile phosphate-buffered saline solution. This colony was then used to inoculate two cultures of M9+KYN, grown into late-logarithmic phase and the optical density was determined at 600 nm. These cultures were then diluted to 10³ in 3 columns of a 96-well deep-well plate with 1 mL of M9+KYNU. Each one of the three rows had different ToxTrp concentrations (increasing 2-fold), while each column had decreasing concentrations of KYN (by 2-fold). Every 12 hours, the plate was diluted back using 30 uL from the well in which the culture had grown to an OD600 of roughly 0.1. This process was repeated for five days, and then the ToxTrp concentrations were doubled to maintain selection pressure. After two weeks' time, no growth rate increases were detected and the culture was plated onto M9+KYN. All culturing was done shaking at 350 RPM at 37° C. Individual colonies were selected and screened in M9+KYN+ToxTrp media to confirm increased growth rate phenotype.

Two replicates for each parental strain (SYN20207-R1, SYN2027-R2, SYN2028-R1, and SYN2028-R2) were selected and assayed for kynurenine production.

Briefly, overnight cultures were diluted 1:100 in 400 ml LB and let grow for 4 hours. Next, 2 ml of the culture was spun down and resuspended in 2 ml M9 buffer. The OD600 of the culture was measured (1/100 dilution in PBS). The necessary amount of cell culture for a 3 ml assay targeting starting cell count of ˜OD 0.8 (˜1E8) was spun down. The cell pellet was resuspended in M9+0.5% glucose+75 uM KYN in the assay volume (3 ml) in a culture tube. 220 ul was removed in triplicate at each time point (t=0, 2, and 3 hours) into conical shaped 96WP, and 4 ul were removed for cfu measurement at each time point. At each time point, the sample was spun down in the conical 96WP for 5 minutes at 3000 g, and 200 ul were transferred from each well into a clear, flat-bottomed, 96WP. A kynurenine standard curve and blank sample was prepared in the same plate. Next, 40 ul of 30% Tri-Chloric Acid (v/v) was added to each well and mixed by pipetting up and down. The plat was sealed with aluminum foil and incubated at 60 C for 15 minutes. The plate was the spun down at 11500 rpm, at 4 C, for 15 minutes, and 125 ul from each well were aliquoted and mixed with 125 ul of 2% Ehrlich's reagent in glacial acetic acid in another 96WP. Samples were mixed pipetting up and down and the absorbance was measured at OD480. Growth rates are shown for parental strains SYN2027 and SYN2028 and the corresponding evolved strains in FIG. 51.

### Example 8. Kynurenine Consuming Strains Decrease Tumoral Kynurenine Levels in the CT26 Murine Tumor Model

The ability of genetically engineered bacteria comprising kynureninase from Pseudomonas fluorescens to consume kynurenine in vivo in the tumor environment was assessed. SYN1704, an E. coli Nissle strain comprising a deletion in Trp:E and a medium copy plasmid expressing kynureninase from Pseudomonas fluorescens under control of a constitutive promoter (Nissle ΔTrpE::CmR+Pconstitutive-Pseudomonas KYNU KanR) was used for in a first study (Study 1).

In a second study (Study 2) the activity of SYN2028, an E. coli Nissle strain comprising a deletion in Trp:E and an integrated construct expressing kynureninase from Pseudomonas fluorescens under the control of a constitutive promoter (Nissle HA3/4::PSynJ23119-pKYNase KanR TrpE::CmR) was assessed.

In both studies, CT26 cells obtained from ATCC were cultured according to guidelines provided. Approximately ˜1e6cells/mouse in PBS were implanted subcutaneously into the right flank of each animal (BalbC/J (female, 8 weeks)), and tumor growth was monitored for approximately 10 days. When the tumors reached about ˜100-150 mm3, animals were randomized into groups for dosing.

For intratumoral injection, bacteria were grown in LB broth until reaching an absorbance at 600 nm (A600 nm) of 0.4 (corresponding to 2×108 colony-forming units (CFU)/mL) and washed twice in PBS. The suspension was diluted in PBS or saline so that 100 microL can be injected at the appropriate doses intratumorally into tumor-bearing mice.

Study 1:

Approximately 10 days after CT 26 implantation, bacteria were suspended in 0.1 nil of PBS and mice were injected (5e6 cells/mouse) with 100 ul intratumorally as follows: Group 1-Vehicle Control (n=8), Group 2-SYN94 (n=8), and Group 3-SYN1704 (n=8). From Day 2 until study end, animals were dosed intratumorally biweekly with 100 ul of vehicle control or bacteria at 5e6 cells/mouse. Animals were weighed and the tumor volume measured twice weekly. Animals were euthanized when the tumors reached ˜2000 mm3 and kynurenine concentrations were measured by LC/MS as described herein. Results are shown in FIG. 52A. A significant reduction in intra tumor concentration was observed for the kynurenine consuming strain SYN1704 and for wild type E. coli Nissle. Intratumoral kynurenine levels were reduced in SYN1704, as compared to wild type Nissle, although the difference did not reach significance due to one outlier.

Study 2:

Approximately 10 days after CT 26 implantation, bacteria were suspended in 0.1 nil of saline and mice were injected (1e8 cells/mouse) with the bacterial suspension intratumorally as follows: Group 1-Vehicle Control (n=10), Group 2-SYN94 (n=10), Group 3-SYN2028 (n=10). Group 5 (n=10) received INCB024360 (IDO inhibitor) via oral gavage as a control twice daily. From Day 2 until study end, animals were dosed intratumorally biweekly with 100 ul of vehicle control or bacteria at 1e8 cells/mouse. Animals were weighed and the tumor volume measured twice weekly. Group 5 received INCB024360 via oral gavage as a control twice daily until study end Animals were euthanized when the tumors reached ˜2000 mm3 Tumor fragments were placed in pre-weighed bead-buster tubes and store don ice for analysis. Kynurenine concentrations were measured by LC/MS as described herein. Results are shown in FIG. 52B. A significant reduction in intra tumor concentration was observed for the kynurenine consuming strain SYN2028 as compared to wild type Nissle or wild type control. Intratumoral kynurenine levels seen in SYN2028 were similar to those observed for the IDO inhibitor INCB024360.

### Example 9. Comparison of In Vitro Efficacy of Chromosomal Insertion and Plasmid-Bearing Engineered Bacterial Strains

To compare the in vitro efficacy between engineered bacterial strains harboring a chromosomal insertion of ArgAfbr driven by an fnr inducible promoter at the malEK locus and strains with a low copy plasmid comprising ArgAfbr driven by an fnr inducible promoter, arginine levels in the media were measured at various time points post anaerobic induction. Additionally, to assess whether auxotrophy for thymidine may have an effect on arginine production efficiency, arginine production of engineered bacterial strains with or without a ThyA deletion, comprising the fnr-ArgAfbr on a low copy plasmid or integrated on the chromosome, were compared.

Overnight cultures were diluted 1:100 in LB and grown with shaking (250 rpm) at 37° C. After 1.5 hrs of growth, the bacteria cultures were induced as follows: (1) bacteria comprising FNR-inducible argA^{fbr} were induced in LB at 37° C. for 4 hrs in anaerobic conditions in a Coy anaerobic chamber (supplying 90% N₂, 5% CO₂, 5% H₂, and 20 mM nitrate) at 37° C.; (2) bacteria comprising tetracycline-inducible argA^{fbr} were induced with anhydrotetracycline (100 ng/mL). After induction, bacteria were removed from the incubator and spun down at maximum speed for 5 min. The cells were resuspended in 1 mL M9 glucose, and the OD₆₀₀ was measured. Cells were diluted until the OD₆₀₀ was between 0.6-0.8. Resuspended cells in M9 glucose media were grown aerobically with shaking at 37 C. 100 μL of the cell resuspension was removed and the OD₆₀₀ is measured at time=0. A 100 μL aliquot was frozen at −20° C. in a round-bottom 96-well plate for mass spectrometry analysis (LC-MS/MS). At each subsequent time point (e.g., 30, 60, and 120 min), 100 μL of the cell suspension was removed and the OD₆₀₀ was measured; a 100 μL aliquot was frozen at −20C in a round-bottom 96-well plate for mass spectrometry analysis. Samples were analyzed for arginine concentrations. At each time point, normalized concentrations as determined by mass spectrometry vs. OD₆₀₀ were used to determine the rate of arginine production per cell per unit time. A summary of the LC-MS/MS method is provided above.

Arginine production at 30, 60, and 120 min post induction was compared between (1) Syn-UCD301 (SYN-UCD304; comprising 4ArgR and argA^{fbr} expressed under the control of a FNR-inducible promoter integrated into the chromosome at the malEK locus), (2) SYN-UCD205 (comprising ΔArgR and arg^{fbr} expressed under the control of a FNR-inducible promoter on a low-copy plasmid), and (3) SYN-UCD206 (comprising ΔArgR and ΔThyA and argA^{fbr} expressed under the control of a FNR-inducible promoter on a low-copy plasmid. SYN-UCD103 was used as is a control Nissle construct and results are shown.

Shown herein are the levels of arginine production of SYN-UCD205, SYN-UCD206, and SYN-UCD301 measured at 0, 30, 60, and 120 minutes. Arginine production was comparable between all three strains, with the greatest arginine production seen with SYN-UCD301 at 120 minutes, indicating that chromosomal integration of FNR ArgA fbr results in similar levels of arginine production as seen with the low copy plasmid strains expressing the same construct, and may even slightly increase the rate of arginine production. SYN-UCD206 exhibited attenuated arginine production as compared to SYN-UCD205 and SYN-UCD-301 (lower arginine levels at 60 minutes), but reached comparable arginine production levels at 120 minutes, indicating that ΔThyA may have a slight attenuating effect on arginine production. No arginine production was detected for the SYN-UCD103 control.

Next, samples were prepared as described above and arginine production at 120 min post induction was compared between (1) SYN-UCD204 (comprising ΔArgR and argA^{fbr} expressed under the control of a tetracycline-inducible promoter on a low-copy plasmid), and (2) SYN-UCD301 (comprising ΔArgR, CmR and argA^{fbr} expressed under the control of a FNR-inducible promoter integrated into the chromosome at the malEK locus), (3) SYN-UCD302 (comprising ΔArgR, ΔThyA, CmR (chloramphenicol resistance) and argAfbr expressed under the control of a FNR-inducible promoter integrated into the chromosome at the malEK locus), and (4) SYN-UCD303 (comprising ΔArgR, ΔThyA, KanR (kanamycin resistance) and argAfbr expressed under the control of a FNR-inducible promoter integrated into the chromosome at the malEK locus).

SYN-UCD106, comprising ΔArgR and ΔThyA was used as is a control Nissle construct. Results are shown in FIG. 42B. As seen in FIG. 42B, arginine production was elevated to between 0.7 and 0.9 umol/1×10⁹ cells, indicating that arginine production is at similar levels in strains bearing ArgAfbr on a plasmid and strains with integrated copies of ArgAfbr.

### Example 10. Comparison of In Vitro Efficacy of Chromosomal Insertion and Plasmid-Bearing Engineered Bacterial Strains

The in vitro efficacy (arginine production from ammonia) in an engineered bacterial strain harboring a chromosomal insertion of ArgAfbr driven by an fnr inducible promoter at the malEK locus, with ΔArgR and a ThyA deletion and no antibiotic resistance was assessed (SYN-UCD303).

Overnight cultures were diluted 1:100 in LB and grown with shaking (250 rpm) at 37° C. After 1.5 hrs of growth, the bacteria cultures were induced in LB at 37° C. for 4 hrs in anaerobic conditions in a Coy anaerobic chamber (supplying 90% N₂, 5% CO₂, 5% H₂, and 20 mM nitrate) at 37° C. After induction, bacteria were removed from the incubator and spun down at maximum speed for 5 min. The cells were resuspended in 1 mL M9 glucose, and the OD₆₀₀ was measured. Cells were diluted until the OD₆₀₀ was between 0.6-0.8. Resuspended cells in M9 glucose media were grown aerobically with shaking at 37 C. 100 μL of the cell resuspension was removed and the OD₆₀₀ is measured at time=0. A 100 μL aliquot was frozen at −20° C. in a round-bottom 96-well plate for mass spectrometry analysis (LC-MS/MS). At each subsequent time point (e.g., 20, 40, 60, 80, 100, and 120 min), 100 μL of the cell suspension was removed and the OD₆₀₀ was measured; a 100 μL aliquot was frozen at −20C in a round-bottom 96-well plate for mass spectrometry analysis. Samples were analyzed for arginine concentrations. At each time point, normalized concentrations as determined by mass spectrometry vs. OD₆₀₀ were used to determine the rate of arginine production per cell per unit time. A summary of the LC-MS/MS method is provided herein. Results are shown in FIG. 43.

### Example 11. Generation of Constructs and Bacteria for Cytokine Secretion

To produce strains capable of secreting immune modulatory polypeptides, e.g., cytokines, such as hIL-12, mIL-12, hIL-15, GMCSF, TNFα, IFN-gamma, CXCL9 and CXCL10, several constructs were designed employing different secretion strategies. Various cytokine constructs were synthesized, and cloned into vector pBR322 for transformation of E. coli. In some embodiments, the constructs encoding the effector molecules are integrated into the genome. In some embodiments, the constructs encoding the effector molecules are on a plasmid, e.g., a medium copy plasmid.

### Example 12. Activity of Kynurenine Consuming Strain in Combination with Systemic Anti-PD-1 and Anti-CTLA-4 in the MC38 Model

The ability of the kynurenine consuming strain SYN2028 to augment the anti-tumor response of combined anti-CTLA4 and anti-PD-1 was assessed in the C57BL/6-MC38 syngeneic tumor model.

To produce cells used in the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with MC38 tumors, and mice injected intratumorally with the kynurenine consuming bacteria and intraperitoneally with anti-CTLA-4 and anti-PD-1 antibodies according to the study design in Table 16. MC38 cells were implanted (1×105/mouse/100 μL) SC into the right flank of each animal on day −9. Tumor growth was monitored; when the tumors reached ˜50-80 mm\hat{ }3 on day 1, mice were randomized into treatment groups as shown in Table 16.

Tumor volumes and body weights were recorded three times in a week with a gap of 1-2 days in between two measurements.

TABLE 16
Study design

| | | Treatment 1 | | | | Treatment 2 | | | | |
|---|---|---|---|---|---|---|---|---|---|---|
| | | Test | | | | Test | | | | Treatment 3 |
| Group | N | Article | Route | Dose | Schedule | Article | Route | Dose | Schedule | Compound |
| 1 | 12 | Anti- | i.p. | 200 ug | Day 1, 4, | Anti- | i.p. | 100 ug | Day 1, 4, | NA |
| | | PD-1 | | | 7 and 10 | CTLA-4 | | | 7 and 10 | |
| | | Isotype | | | | Isotype | | | | |
| | | Control | | | | Control | | | | |
| 2 | 12 | Anti- | i.p. | 200 ug | Day 1, 4, | Anti- | i.p. | 100 ug | Day 1, 4, | NA |
| | | PD-1 | | | 7 and 10 | CTLA-4 | | | 7 and 10 | |
| 3 | 12 | Anti- | i.p. | 200 ug | Day 1, 4, | Anti- | i.p. | 100 ug | Day 1, 4, | SYN094, |
| | | PD-1 | | | 7 and 10 | CTLA-4 | | | 7 and 10 | 5e6 bacteria, |
| | | | | | | | | | | i.t., BIWx2 |
| 4 | 12 | Anti- | i.p. | 200 ug | Day 1, 4, | Anti- | i.p. | 100 ug | Day 1, 4, | SYN2028, |
| | | PD-1 | | | 7 and 10 | CTLA-4 | | | 7 and 10 | 5e6 bacteria, |
| | | | | | | | | | | i.t, BIWx2 |

Results in FIGS. 58A, 58B, 58C, and 58E show that the kynurenine consuming strain has the ability to improve anti-CTLA-4/anti-PD-1 antibody-mediated anti-tumor activity in the MC38 model. Specifically, in the anti-PD-1/anti-CTLA-4 group, 25% of mice responded to the treatment; same response rate was observed with anti-PD-1/anti-CTLA-4 plus SYN94 group. In the anti-PD-1/anti-CTLA-4 plus SYN2028 group, 71% of mice responded.

### Example 13. Activity of Arginine Producer and Kynurenine Consumer in Combination with Nonmyeloablative Chemotherapy

The activity of the arginine producer (SYN828) and kynurenine consumer (SYN2028) was assessed in combination with cyclophosphamide treatment in the CT26 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with CT26 tumors, and mice injected intratumorally with bacteria producing arginine (SYN828) and consuming kynurenine (SYN2028) and controls in combination with cyclophosphamide (CP) at 100 mg/kg, according to the time line described below and in FIG. 44A.

Briefly, CT26 cells were implanted (1e6 cells/mouse in PBS) SC into the right flank of each animal on day −9. Tumor growth was monitored until the tumors reached ˜50-80 mm\hat{ }3. On day 0, mice were pre-treated with cyclophosphamide at 100 mg/kg (100 uL/mouse) I.P. and randomized into groups. On Day 1 of treatment mice received either SYN94 (WT, 1e8 CFU/mL) (I.T.), SYN2028 (Kyn, 1e8 CFU/mL), or SYN825 (Arg, 1e8 CFU/mL) (I.T.) in 100 uL. On Day 4, 8, 11, and 15, animals were weighed, tumor was measured, and mice were dosed mice with the appropriate treatment/group. Tumor volumes for individual mice are shown in FIGS. 44B, 44C, 44D, 44E, and 44F. and indicate Anti-tumor activity of the kynurenine-consuming and arginine producing strains in combination with cyclophosphamide over cyclophosphamide alone.

### Example 14. Cyclic-Di-AMP Quantitation in Bacterial Cell Pellet and Tumor Tissue Homogenate by LC-MS/MS

Sample Preparation

To generate the standards, 10 mg/mL of Cyclic-di-AMP was prepared in 1.5 mL microcentrifuge tubes, and 250, 100, 20, 4, 0.8, 0.16, 0.032 μg/mL solutions were prepared in water. QC solutions were prepared with 200, 20, and 2 μg/mL levels.

Sample Preparation: for in vitro samples, bacterial pellet was extracted by adding 100 uL of 2:1 acetonitrile:water, and vortexed and centrifuged. 20 μL of supernatant was transferred to a new 96 well plate and diluted tenfold by adding 180 μL 0.1% formic acid. For in vivo sample, tissue homogenates were extracted by adding 90 μL of 2:1 acetonitrile: water to 10 μL tumor homogenate. Samples were vortexed and centrifuged. 20 μL of supernatant was transferred to a new 96 well plate and diluted tenfold by adding 180 μL 0.1% formic acid. Plates were heat-sealed with a ClearASeal sheet and mixed well.

### LC-MS/MS Method

Analytes were measured by liquid chromatography coupled to tandem mass spectrometry (LC-MS/MS) using a Thermo TSQ Quantum Max triple quadrupole mass spectrometer. Table 17-Table 19 provide the summary of the LC-MS/MS method.

TABLE 17

| | |
|---|---|
| Column: | Thermo Accucore aQ C18 2.6 μm (100 × 2.1 mm) |
| Mobile Phase A: | 100% H2O, 0.1% Formic Acid |
| Mobile Phase B: | 100% ACN, 0.1% Formic Acid |
| Injection volume: | 10 μL |

TABLE 18
HPLC Method

| Time (min) | Flow Rate (μL/min) | A % | B % |
|---|---|---|---|
| 0 | 300 | 100 | 0 |
| 0.5 | 300 | 100 | 0 |
| 1.0 | 300 | 10 | 90 |
| 2.5 | 300 | 10 | 90 |
| 2.51 | 300 | 100 | 0 |
| 4.0 | 300 | 100 | 0 |

TABLE 19
Tandem Mass Spectrometry:

| | |
|---|---|
| Ion Source: | HESI-II |
| Polarity: | Positive |

Analyte SRM transitions:

| | |
|---|---|
| Cyclic-di-AMP: | 659.4 > 329.6 |

For data analysis, SRM chromatograms were integrated and peak areas of the standards were plotted against concentration. A linear curve was fit and concentrations of the unknowns were calculated using their peak areas and the slope intercept form equation from the standard curve.

### Example 15. Display of Anti-mPD1-scFv on E. coli Nissle Cell Surface

To generate genetically engineered bacteria which are capable of displaying anti-mPD1-scFv on the Nissle cell surface, constructs were generated according to methods described herein as shown in Table 20. Sequences are SEQ ID NO: 987-989. Display anchor polypeptides include SEQ ID NO: 990-992.

TABLE 20
Strains for display of anti-mPD1-scFv

| Strain | Strain | |
|---|---|---|
| Number | Genotype | Construct |
| SYN2797 | wt Nissle | p15A-Kan-ptet-Invasin-FLAG-J43scFv- |
| | | V5-HIS |
| SYN2798 | wt Nissle | p15A-Kan-ptet-LppOmpA-FLAG-J43scFv- |
| | | V5-HIS |
| SYN2799 | wt Nissle | p15A-Kan-ptet-IntiminN-FLAG-J43scFv- |
| | | V5-HIS |

In some embodiments, the display anchor is at least about 80%, at least about 85%, at least about 90%, at least about 95%, or at least about 99% homologous to the sequence of SEQ ID NO: 990, SEQ ID NO: 991, and/or SEQ ID NO: 992.

E. coli Nissle comprising a plasmid based construct comprising tet-inducible ptet-LppOmpA-anti-PD1-scFv was grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression of ptet-LppOmpA-J43-scFv for 18 hours.

To determine whether the single-chain antibody was displayed on the surface of the genetically engineered E. coli Nissle and functionally binds to PD1 a whole cell ELISA assay was performed. 10\hat{ }9 cells were blocked using PBS with 2% BSA for 1 h at room temperature and biotinylated-mPD1 was added and incubated for 1 h at room temperature. Afterwards, cells were washed 3 times with PBST (PBS/0.1% Tween-20) and incubated with a streptavidin conjugated HRP in blocking solution for 40 min. Following incubation, wells were washed 3 times with PBST and resuspended in PBS, then stained using a 3,3′,5,5′-tetramethylbenzidine (TMB) substrate kit per the manufacturer's instructions (Thermofisher). Biotinylated IgG and plain PBS were used instead of mPD1 as negative controls. Cells were removed by centrifugation and supernatants were collected. Signal intensities of supernatant were measured using an ELISA reader at 450 nm. Results (data not available) indicate that the J43-scFv (anti-mPD1) is displayed on the surface of the genetically engineered bacteria and can bind to mPD1 (Table 21).

TABLE 21
Nissle Surface Display ELISA Assay

| | | Primary | Secondary |
|---|---|---|---|
| Strain | OD450 | antibody | antibody |
| SYN2798 (p15A-ptet-LppOmpA-anti- | 0.125 | PBS only | Strp-HRP |
| PD1-scFv) | | | |
| SYN2798 (p15A-ptet-LppOmpA-anti- | 0.133 | mIgG-strp | Strp-HRP |
| PD1-scFv) | | | |
| SYN2798 (p15A-ptet-LppOmpA-anti- | 0.421 | mPD1-strp | Strp-HRP |
| PD1-scFv) | | | |

### Example 16. α-PD1-scFv Expression in E. coli

To determine whether a functional scFv can be expressed in E. coli, an anti-PD1-scFv fragment was generated based on J43 monoclonal antibody, which reacts with mouse PD-1.

Mouse monoclonal antibody J43 sequence was obtained from patent EP 1445264 A1. Next, the single-chain variable fragment (scFv) was designed. A fragment containing tet promoter, a ribosome binding site, the designed J43-scFv, a C terminal V5 tag and a C terminal hexa-histidine tag (SEQ ID NO: 1252) was synthesized by IDTDNA. The construct was cloned into the pCR™-Blunt II-TOPO® Vector (Invitrogen) and transformed into E. coli DH5a as described herein to generate the plasmid pUC-ptet-J43scFv-V5-HIS (SEQ ID NO: 976-980).

E. coli comprising either tet-inducible J43-Anti-PD1-scFv-V5 or wild type controls were grown overnight in LB medium. Cultures were diluted 1:40 in LB and grown shaking (250 rpm) to an optical density of 0.8 at which time anhydrous tetracycline (ATC) was added to cultures at a concentration of 100 ng/mL to induce expression of J43-Anti-PD1-scFv-V5. Same amount of tetracycline was added to wild type control cultures. After 4 hrs of induction, bacteria were pelleted, washed in PBS, and harvested, resuspended in 2 mL sonication buffer (PBS), and lysed by sonication on ice. Insoluble debris was spun down twice for 15 min at 12,000 rpm at 4° C.

Protein concentration was determined by BCA protein assay, and isolated extracts from wild type and strains comprising the Ptet-J43-Anti-PD1-scFV-V5 were analyzed by Western blot. Proteins were transferred onto PVDF membranes and J43-Anti-PD1-scFv was detected with an HRP-conjugated anti-V5 antibody (Biolegend). A single band was detected at 27 kDa in lane 2 (extract from J43-Anti-PD1-scFv-V5 strain). No bands were detected in lane 1 (wild type extract).

To determine whether the single-chain antibody purified from E. coli DH5a functionally binds to the target protein, PD1, an ELISA assay was performed. Plates were absorbed overnight at 4° C. with 100 μL of 2 μg/mL per well of PD1 (Rndsystems). Wells were blocked with 2% BSA in PBS/0.1% Tween-20 for 2 hours at room temperature. After three washes, wells were incubated with bacterial extracts (J43-scFv-V5 or wild type-neg-ctrl) for 1 hour at room temperature. Wells were washed 4 times with PBST (PBS/0.1% Tween-20) and incubated with a HRP-conjugated anti-V5 antibody (Biolegend) in blocking solution for 40 min. Following incubation, wells were washed 4 times with PBST and then stained using a 3,3′,5,5′-tetramethylbenzidine (TMB). Signal intensities were measured using an ELISA reader at 450 nm. Results are shown in Table 22 and indicate that the antibody expressed by the genetically engineered bacteria can bind to PD1 specifically.

TABLE 22
ELISA Binding Assay

| 1′ | PBS | mPD1 | IgG | 2′ |
|---|---|---|---|---|
| antibody | coating | coating | coating | antibody |
| Wild type-neg-ctrl | 0.11 | 0.13 | 0.12 | α-V5-HRP |
| J43-scFv-V5 | 0.11 | 1.41 | 0.13 | α-V5-HRP |
| Wild type-neg-ctrl (1/2) | 0.10 | 0.09 | 0.10 | α-V5-HRP |
| J43-scFv-V5 (1/2) | 0.10 | 0.90 | 0.11 | α-V5-HRP |

Next, recombinant J43-Anti-scFv-V5 was expression using pET22b vector harvesting a C-terminal poly-histidine tag and purified using immobilized metal ion affinity chromatography. Protein concentration was determined by absorption at 280 nm and purity was confirmed by Coomassie gel (data not shown).

To determine whether anti-PD1-scFv expressed in E. coli binds to surface PD1 on mouse EL4 cells, flow cytometric analysis was performed using EL4 cells. EL4 are a mouse lymphoma cell line which expresses PD1 on its cell surface.

EL4 cells were grown in Dulbecco's Modified Eagle's Medium (DMEM) with 10% FBS. Cells were spun down, supernatant was aspirated, pellet was resuspended in 1 nil D-PBS, transferred into chilled assay tubes (1×106 cells), and washed 2-3 times in D-PBS with 0.5% BSA. Cells were resuspended in PBS with 0.5% BSA, to which the purified scFv-V5 and anti-V5-FITC antibody were added and incubated for 1 hour at room temperature. Negative control left out scFv-V5. Cells were resuspended in 0.5 ml PBS and analyzed on a flow cytometer. Results are shown in FIG. 62. A population shift is observed only when the purified anti-PD1-scFv-V5 and anti-V5-FITC were both present (two different batches were shown), relative to samples with EL4 alone and EL4 plus secondary antibody only.

### Example 17. Secretion of Anti-mPD1-scFv

Strains generated according to methods described herein for secretion of anti-mPD1-scFv are shown in Table 24.

TABLE 23
Strains for secretion of anti-mPD1-scFv

| Strain Number | Genotype | Construct |
|---|---|---|
| SYN2790 | Nissle Δ nlpI::CmR | pUC-ptet-OmpF-FLAG- |
| SYN2767 | Nissle Δ tolA::CmR | pUC-ptet-OmpF-FLAG- |
| SYN2768 | Nissle Δ PAL::CmR | pUC-ptet-OmpF-FLAG- |
| SYN2769 | Nissle Δ lpp::CmR | pUC-ptet-OmpF-FLAG- |
| SYN2770 | Nissle Δ nlpI::CmR | pUC-ptet-PhoA-FLAG- |
| SYN2771 | Nissle Δ tolA::CmR | pUC-ptet-PhoA-FLAG- |
| SYN2772 | Nissle Δ PAL::CmR | pUC-ptet-PhoA-FLAG- |
| SYN2773 | Nissle Δ lpp::CmR | pUC-ptet-PhoA-FLAG- |
| SYN2774 | Nissle Δ nlpI::CmR | pUC-ptet-PelB-FLAG- |
| SYN2775 | Nissle Δ tolA::CmR | pUC-ptet-PelB-FLAG- |
| SYN2776 | Nissle Δ PAL::CmR | pUC-ptet-PelB-FLAG- |
| SYN2777 | Nissle Δ lpp::CmR | pUC-ptet-PelB-FLAG- |

E. coli Nissle comprising plasmid based construct comprising tet-inducible J43-Anti-scFv-V5 with PhoA, OmpF or PelB secretion tags (see SEQ ID NOs: 981-986) or wild type control were grown overnight in LB medium. Cultures were diluted 1:100 in LB and grown shaking (200 rpm) to an optical density of 0.8 at which time cultures were cooled down to room temperature and anhydrous tetracycline (ATC) was added to cultures at a concentration of 100 ng/mL to induce expression of PhoA-, OmpF- or PelB-J43-Anti-scFv-V5. No tetracycline was added to wild type Nissle cultures. After 18 hrs of induction at room temperature, bacteria were pelleted, and the supernatant was collected and placed on ice.

Protein concentration in the medium and the cell lysates was determined by BCA protein assay, and isolated extracts and media from wild type and strains comprising the Ptet-J43-anti-scFv-V5 were analyzed by Western blot. Proteins were transferred onto PVDF membranes and J43-anti-scFv detected with an HRP-conjugated anti-V5 antibody (Biolegend). Results are shown in FIG. 63. A single band was detected around 34 kDa in lane 1-6 corresponding to extracts from SYN2767, SYN2769, SYN2771, SYN2773, SYN2775 and SYN2777 respectively.

To determine whether the secreted J43-anti-scFv in E. coli Nissle binds to PD1 on mouse cells, flow cytometric analysis was performed using EL4 cells. EL4 are a mouse lymphoma cell line which expresses PD1 on its cell surface.

EL4 cells were grown in Dulbecco's Modified Eagle's Medium (DMEM) with 10% FBS and 1% Penicillin-Streptomycin Cells were spun down, supernatant was aspirated, pellet was resuspended in 1 ml D-PBS, transferred into chilled assay tubes (1×10\hat{ }6 cells), and washed 3 times in D-PBS. Cells were resuspended in D-PBS with 0.5% BSA, to which the purified scFv-V5 and anti-V5-FITC antibody were added and incubated for 1 hour at room temperature. Negative control left out secreted J43-scFv-V5. Cells were then resuspended in 0.5 ml PBS and analyzed on a flow cytometer. Results are shown in (FIG. 64). A population shift is observed only when the secreted anti-PD1-scFv-V5 (1′ antibody) and anti-V5-FITC (2′ antibody) were both present, relative to samples with EL4 alone and EL4 plus secondary antibody only. A similar study was conducted with different amounts of the secreted scFv (0, 2, 5, and 15 tit), and a dose-dependent staining of the EL4 cells was observed (FIG. 65).

Next, a competition assay was conducted to determine whether PDL1 could inhibit the binding of the anti-PD1-scFv secreted by the genetically engineered bacteria from binding to murine PD1. EL4 cells were grown and flow cytometry protocol was conducted essentially as described above except that PDL1 was added at various concentrations (0, 5, 10, and 30 mg/mL) during the incubation of the secreted anti-PD1-scFv-V5. Rat-IgG was used as a negative control of secreted scFv. Results are shown in FIG. 66A and FIG. 66B. PDL1 competed in a dose dependent manner against the binding of secreted anti-mPD1-scFv to mPD1 on the surface of EL4 cells. Negative control of Rat-IgG protein did not show similar dose dependent binding competition.

### Example 18. Cytokine Secretion (IL-15)

To determine whether the hIL-15 expressed by engineered bacteria is secreted, the concentration of hIL-15 in the bacterial supernatant from engineered strains comprising hIL-15 secretion constructs/strains was measured. The strains comprise either a deletion in Lpp (lpp::Cm), nlpl (nlpL::Cm), tolA (tolA::Cm), or PAL (PAL::Cm). All strains further comprise a plasmid expressing hIL-15 with a PhoA secretion tag.

E. coli Nissle strains were grown overnight in LB medium. Cultures were diluted 1:200 in LB and grown shaking (200 rpm) for 2 hours. Cultures were diluted to an optical density of 0.5 at which time anhydrous tetracycline (ATC) was added to cultures at a concentration of 100 ng/mL to induce expression of hIL-15. After 12 hours of induction, cells were spun down, and supernatant was collected. To generate cell free medium, the clarified supernatant was further filtered through a 0.22 micron filter to remove any remaining bacteria and placed on ice. Additionally, to detect intracellular recombinant protein production, pelleted were bacteria washed and resuspended in BugBuster™ (Millipore) with protease inhibitors and Ready-Lyse Lysozyme Solution (Epicenter), resulting in lysate concentrated 10-fold compared to original culture conditions. After incubation at room temperature for 10 minutes insoluble debris is spun down at 20 min at 12,000 ref at 4.0 then placed on ice until further processing.

The concentration of hIL-15 in the cell-free medium and in the bacterial cell extract was measured by hIL-15 ELISA (RnD Systems, Minneapolis, Minn.), according to manufacturer's instructions. All samples were run in triplicate, and a standard curve was used to calculate secreted levels of hIL-15. Standard curves were generated using recombinant hIL-15. Wild type Nissle was included in the ELISA as a negative control, and no signal was observed. Table 24 summarizes levels of hIL-15 measured in the respective supernatants. The data show that hIL-15 is secreted at various levels from the different bacterial strains.

TABLE 24
Concentration of Secreted hIL-15

| | | | [IL-15] (ng/ml) |
|---|---|---|---|
| ID | Genotype | Construct | in the medium |
| SYN1817 | Lpp (lpp::Cm) | pBR322.Ptet.phoA-IL15 | 27.9 |
| SYN1818 | nlpI (nlpI::Cm) | pBR322.Ptet.phoA-IL15 | 30.4 |
| SYN1819 | tolA (tolA::Cm) | pBR322.Ptet.phoA-IL15 | 33.8 |
| SYN1820 | PAL (PAL::Cm) | pBR322.Ptet.phoA-IL15 | 38.0 |

### Example 19. Cytokine Secretion (GMCSF)

To determine whether hGMCSF expressed by engineered bacteria is secreted, the concentration of hGMCSF in the bacterial supernatant from engineered strains comprising hGMCSF secretion constructs/strains was measured. The strains comprise either a deletion in Lpp (lpp::Cm), nlpl (nipL::Cm), tolA (tolA::Cm), or PAL (PAL::Cm). All strains further comprise a plasmid expressing hGMCSF with a PhoA secretion tag.

E. coli Nissle strains were grown, induced and processed as described in the previous example for IL-15.

The concentration of hGMCSF in the cell-free medium and in the bacterial cell extract was measured by hGMCSF ELISA (RnD Systems, Minneapolis, Minn.), according to manufacturer's instructions. All samples were run in triplicate, and a standard curve was used to calculate secreted levels of hGMCSF. Standard curves were generated using recombinant hGMCSF. Wild type Nissle was included in the ELISA as a negative control, and no signal was observed. Table 25 summarizes levels of hGMCSF measured in the respective supernatants. The data show that hGMCSF is secreted at various levels from the different bacterial strains.

TABLE 25
Concentration of Secreted GMCSF

| | | | | [GMCSF] (ng/ml) | [GMCSF] (ng/ml) |
|---|---|---|---|---|---|
| | | | | in the medium | in the medium |
| ID | Genotype | High copy construct | Low copy construct | High copy plasmid | Low copy plasmid |
| SYN094 | WT | None | None | 0.0 | 0.0 |
| SYN2036/ | lpp | pUC.Ptet.phoA- | pUN UNSX-TetR-Ptet- | 45.8 | 44.7 |
| SYN2093 | | GMCSF | phoA-GMCSF-UNS9 | | |
| SYN2038/ | PAL | pUC.Ptet.phoA- | pUN UNSX-TetR-Ptet- | 114.3 | 98.8 |
| SYN2103 | | GMCSF | phoA-GMCSF-UNS9 | | |
| SYN2037/ | nlpI | pUC.Ptet.phoA- | pUN UNSX-TetR-Ptet- | 39.9 | 44.0 |
| SYN2095 | | GMCSF | phoA-GMCSF-UNS9 | | |

### Example 20. Cytokine Secretion (TNFa)

To determine whether hTNFa expressed by engineered bacteria is secreted, the concentration of hTNFa in the bacterial supernatant from engineered strains comprising hTNFa secretion constructs/strains was measured. The strains comprise either a deletion in Lpp (lpp::Cm), nlpI (nlpL::Cm), tolA (tolA::Cm), or PAL (PAL::Cm). All strains further comprise a plasmid expressing hTNFa with a PhoA secretion tag.

E. coli Nissle strains were grown, induced and processed as described in the previous example for IL-15.

The concentration of hTNFa in the cell-free medium and in the bacterial cell extract was measured by hTNFa ELISA (RnD Systems, Minneapolis, Minn.), according to manufacturer's instructions. All samples were run in triplicate, and a standard curve was used to calculate secreted levels of hTNFa. Standard curves were generated using recombinant hTNFa. Wild type Nissle was included in the ELISA as a negative control, and no signal was observed. Table 26 summarizes levels of hTNFa measured in the respective supernatants. The data show that hTNFa is secreted at various levels from the different bacterial strains.

TABLE 26
Concentration of Secreted TNFa

| | | | Secreted |
|---|---|---|---|
| Strain | Genotype | Construct | [TNFa] ng/mL |
| SYN094 | WT | None | 0 |
| SYN2541 | lpp::Cm | Nissle Ptet-phoA- | 129.6 |
| | | TNFa | |
| SYN2542 | nlpI::Cm | Nissle Ptet-phoA- | 345.3 |
| | | TNFa | |
| SYN2543/ | PAL::Cm | Nissle Ptet-phoA- | >400 |
| SYN2304 | | TNFa | |
| SYN2544 | TrpE PAL::Cm | HA3/4::Plpp- | >400 |
| | | pKYNase Ptet-phoA- | |
| | | TNFa | |
| SYN2545 | TrpE PAL::Cm | HA3/4::PSyn- | >400 |
| | | pKYNase Ptet-phoA- | |
| | | TNFa | |

### Example 21. Functional Assay for Secreted TNFα

Next, studies were conducted to demonstrate that TNFα secreted from genetically engineered bacteria is functional. A cell-based assay was employ based on TNFα mediated NF-kappaB activation. TNFα binding to its receptor binding results in phosphorylation and degradation of IkBalpha by IKK. Bioactivity of TNFα can be determined by quantification of IkB degradation via flow cytometry.

Briefly, HeLa cells were treated with TNFα secreting supernatants derived from SYN2304 (comprising PAL::Cm p15a TetR Ptet-phoA TNFα) for 10 min. Cells were then fixed in paraformaldehyde based buffer followed by permeabilization in Triton X-100. Modulation of IkBa degradation determined by flow cytometry, and results are shown in FIG. 37.

As seen in FIG. 37, SYN2304 exhibits bioactivity approaching that of rhTNFa, and SYN1557 treatment does not result in measurable signal indicating no confounds from off target components of bacterial supernatants (ie LPS).

### Example 22. Cytokine Secretion (hIFNg)

To determine whether hIFNg expressed by engineered bacteria is secreted, the concentration of hIFNg in the bacterial supernatant from engineered strains comprising hTNFa secretion constructs/strains was measured. The strains comprise either a deletion in Lpp (lpp::Cm), nlpI (nlpI::Cm), tolA (tolA::Cm), or PAL (PAL::Cm). All strains further comprise a plasmid expressing hIFNg with a PhoA secretion tag.

E. coli Nissle strains were grown, induced and processed as described in the previous example for IL-15.

The concentration of hIFNg in the cell-free medium and in the bacterial cell extract was measured by hIFNg ELISA (RnD Systems, Minneapolis, Minn.), according to manufacturer's instructions. All samples were run in triplicate, and a standard curve was used to calculate secreted levels of hIFNg. Standard curves were generated using recombinant hIFNg. Wild type Nissle was included in the ELISA as a negative control, and no signal was observed. Table 27 summarizes levels of hIFNg measured in the respective supernatants. The data show that hIFNg is secreted at various levels from the different bacterial strains.

TABLE 27
Concentration of Secreted IFNg

| | | | Secreted |
|---|---|---|---|
| Strain | Genotype | Construct | [IFNg] ng/mL |
| SYN094 | WT | None | 0 |
| SYN2546 | lpp::Cm | Nissle Ptet-phoA-IFNg | 44.9 |
| SYN2547 | nlpI::Cm | Nissle Ptet-phoA-IFNg | 51.5 |
| SYN2548 | PAL::Cm | Nissle Ptet-phoA-IFNg | 85.9 |
| SYN2549 | TrpE PAL::Cm | HA3/4::Plpp-pKYNase | 39.1 |
| | | Ptet-phoA-IFNg | |
| SYN2550 | TrpE PAL::Cm | HA3/4::PSyn-pKYNase | 87.6 |
| | | Ptet-phoA-IFNg | |

Table 28 provides a summary of the levels of secretion obtained for each cytokine, and lists some structural features of the cytokine which may explain some of the differences in secretion levels observed.

TABLE 28
Summary of Secretion Results

| | | | | | | Secretion |
|---|---|---|---|---|---|---|
| | Size | | O-linked | N-linked | Disulphide | level |
| Therapeutic | (Dal) | Stoichiometry | Glycosylation | Glycosylation | Bonds | (ng/mL) |
| hIL-15 | 14715 | Monomer | 0 | 1 | 2 | 38.0 |
| GMCSF | 14477 | Monomer | 4 | 2 | 2 | 114.0 |
| TNFα | 17353 | Monomer | 1 | 0 | 1 | >400 |
| IFN-gamma | 16177 | Homodimer | 0 | 2 | 0 | 87.6 |

### Example 23. Anti-CD47 scFv Expression in E. coli

To determine whether a functional anti-CD47-scFv can be expressed in E. coli, an anti-CD47-scFv fragment was generated based on B6H12 and 5F9 monoclonal antibodies, which reacts with human CD47.

Monoclonal antibody B6H12 and 5F9 (anti human CD47) sequences were obtained from published patent (US 20130142786 A1). Next, the single-chain variable fragment (scFv) targeting human CD47 was designed. A fragment containing tet promoter, a ribosome binding site, the designed antihCD47-scFv, a C terminal V5 tag and a C terminal hexa-histidine tag (SEQ ID NO: 1252) was synthesized by IDTDNA. The construct was cloned into the pCR™-Blunt II-TOPO® Vector (Invitrogen) and transformed into E. coli DH5a as described herein to generate the plasmid pUC-ptet-B6H12antihCD47scFv-V5-HIS (SEQ ID NO: 993) and pUC-ptet-5F9antihCD47scFv-V5-HIS (SEQ ID NO: 994).

E. coli DH5a comprising pUC-ptet-B6H12antihCD47scFv-V5-HIS or pUC-ptet-5F9antihCD47scFv-V5-HIS were grown overnight in LB medium. Cultures were diluted 1:100 in LB and grown shaking (200 rpm) to an optical density of 0.8 at which time cultures were cooled down to room temperature and anhydrous tetracycline (ATC) was added to cultures at a concentration of 100 ng/mL to induce expression of ptet-scFv for 18 hours and then bacteria were pelleted, washed in PBS, and harvested, resuspended in 2 mL PBS buffer and lysed by sonication on ice. Insoluble debris is spun down twice for 15 min at 12,000 rpm at 4.C.

To determine whether the anti CD47 single-chain antibody expressed in E. coli DH5a functionally binds to the target protein, an ELISA assay was performed. Plates were absorbed overnight at 4° C. with 100 μL of 2 μg/mL per well of target proteins (humanCD47, mouseCD47, IgG and PBS, from Rndsystems). Wells were blocked with 2% BSA in PBS/0.1% Tween-20 for 2 hours at room temperature. After three washes, wells were incubated with bacterial extracts for 1 hour at room temperature. Wells were washed 4times with PBST (PBS/0.1% Tween-20) and incubated with a HRP-conjugated anti-V5 antibody(Biolegend) in blocking solution for 40 min. Following incubation, wells were washed 4 times with PBST and then stained using a 3,3′,5,5′-tetramethylbenzidine (TMB). Signal intensities were measured using an ELISA reader at 450 nm. Results are shown in Table 29 and indicate that the antiCD47-scFv expressed by the genetically engineered bacteria can bind to humanCD47 specifically.

TABLE 29
ELISA Binding Assay

| | | Primary | Secondary | |
|---|---|---|---|---|
| Strain | Coating | antibody | antibody | OD450 |
| SYN2936 (pUC-Ptet- | PBS | B6H12-scFv | Anti-V5-HRP | 0.047 |
| B6H12scFv-V5-HIS) | | extracts | | |
| SYN2936 (pUC-Ptet- | IgG | B6H12-scFv | Anti-V5-HRP | 0.064 |
| B6H12scFv-V5-HIS) | | extracts | | |
| SYN2936 (pUC-Ptet- | hCD47 | B6H12-scFv | Anti-V5-HRP | 1.587 |
| B6H12scFv-V5-HIS) | | extracts | | |
| SYN2936 (pUC-Ptet- | mCD47 | B6H12-scFv | Anti-V5-HRP | 0.053 |
| B6H12scFv-V5-HIS) | | extracts | | |
| SYN2937 (pUC-Ptet- | PBS | 5F9-scFv | Anti-V5-HRP | 0.048 |
| 5F9scFv-V5-HIS) | | extracts | | |
| SYN2937 (pUC-Ptet- | IgG | 5F9-scFv | Anti-V5-HRP | 0.057 |
| 5F9scFv-V5-HIS) | | extracts | | |
| SYN2937 (pUC-Ptet- | hCD47 | 5F9-scFv | Anti-V5-HRP | 1.838 |
| 5F9scFv-V5-HIS) | | extracts | | |
| SYN2937 (pUC-Ptet- | mCD47 | 5F9-scFv | Anti-V5-HRP | 0.053 |
| 5F9scFv-V5-HIS) | | extracts | | |

### Example 24. Kynurenine Consuming Strains Decrease Tumoral Kynurenine Levels in the CT26 Murine Tumor Model

The ability of genetically engineered bacteria comprising kynureninase from Pseudomonas fluorescens to consume kynurenine in vivo in the tumor environment was assessed. SYN1704, an E. coli Nissle strain comprising a deletion in Trp:E and a medium copy plasmid expressing kynureninase from Pseudomonas fluorescens under control of a constitutive promoter (Nissle deltaTrpE::CmR+Pconstitutive-Pseudomonas KYNU KanR) was used.

In both studies, CT26 cells obtained from ATCC were cultured according to guidelines provided. Approximately ˜1e6 cells/mouse in PBS were implanted subcutaneously into the right flank of each animal (BalbC/J (female, 8 weeks)), and tumor growth was monitored for approximately 10 days. When the tumors reached about ˜100-150 mm³, animals were randomized into groups for dosing.

For intratumoral injection, bacteria were grown in LB broth until reaching an absorbance at 600 nm (A600 nm) of 0.4 (corresponding to 2×10⁸ colony-forming units (CFU)/mL) and washed twice in PBS. The suspension was diluted in PBS or saline so that 100 uL can be injected at the appropriate doses intratumorally into tumor-bearing mice.

Approximately 10 days after CT 26 implantation, bacteria were suspended in 0.1 mL of PBS and mice were injected (1e7 cells/mouse) with 100 uL intratumorally as follows: Group 1-Saline Control (n=7), Group 2-SYN1704 (n=7), Animals were dosed bi-weekly (BIW) according to their grouping either with saline or with the strains intratumorally (IT) Animals were weighed and the tumor volume measured twice weekly. Animals were euthanized when the tumors reached ˜2000 mm³. Plasma and tumor tissue were harvested and kynurenine and tryptophan concentrations were measured by LC/MS as described herein. Results are shown in FIGS. 42A and 42B. A significant reduction in intratumoral (P<0.001) and plasma (P<0.005) concentration of kynurenine was observed for the kynurenine consuming strain SYN1704. Tryptophan levels remained constant (data not shown).

### Example 25. Kinetic Study with SYN94 and SYN1704 in a CT26 Tumor Model

The effects of single administration of a KYN-consuming strain in CT26 tumors has on tumoral KYN levels and tumor weight was assessed. Female Balb/C mice (18-25 g) from CRL at 8wks of age were allowed to acclimate to facility for at least 3 days Animals were being placed on regular food and water.

On day −8, CT26 cells (˜1e6 cells/mouse in PBS) were implanted SC into right flank of each animal. Tumor growth was monitored for one week until tumors reached ˜100-150 mm3 Animals were then weighed, measured and randomized into treatment groups according to Table 30 (Day 1) Animals were dosed with SYN94 or SYN1704 at the appropriate concentration via intratumoral dosing. For intratumoral injection, SYN94 cells were diluted from a 3.0×10e11 CFU/mL stock to a concentration of 1×10e8 CFU/mL and SYN1704 cells were diluted from a stock to a concentration of 1×10e8 CFU/mL.

TABLE 30
Study Design

| | Number of | | | | | |
|---|---|---|---|---|---|---|
| Group | animals/ | Test | Concen- | Dose | | Dose |
| Number | sex | Article | tration | Volume | Media | Route |
| 1 | 4/F | None | None | 100 μL | None | None |
| 2 | 4/F | SYN94 | 1e8 CFU/mL | 100 μL | PBS | IT |
| 3 | 4/F | SYN94 | 1e8 CFU/mL | 100 μL | PBS | IT |
| 4 | 4/F | SYN94 | 1e8 CFU/mL | 100 μL | PBS | IT |
| 5 | 4/F | SYN1704 | 1e8 CFU/mL | 100 μL | PBS | IT |
| 6 | 4/F | SYN1704 | 1e8 CFU/mL | 100 μL | PBS | IT |
| 7 | 4/F | SYN1704 | 1e8 CFU/mL | 100 μL | PBS | IT |

Animals were observed daily after dosing for signs of abnormalities or excessive pain associated with tumor growth. Animals were sacrificed on day 1 (T=0 group; Group 1), day 2 (T=24h; Groups 2&5). day 4 (T=72h; Groups 3&6), and day 8 (T=168h; Groups 4&7).

Whole blood was collected for each group via cardiac bleed at appropriate endpoint. Maximum obtainable of blood was collected into LiHep tubes (BD). Samples were kept on ice until they were spun in a centrifuge (2000 g for 10 min at 4C). Plasma was then transferred into 1.5 mL Eppendorf tubes and stored at −80 C until later analysis. Tumor samples were split into two parts, and the first part was collected at appropriate endpoint in reweighed bead buster tubes. Tissues were weighed in tubes before being stored at −80 C for further analysis. The other part of the tumor was fixed in 10% formalin for sectioning and analysis. Plasma samples from blood collection were analyzed by LCMS and cytokine analysis was conducted using cell based assays. Tumor samples were analyzed by LCMS for kynurenine levels. Results are shown in FIGS. 54A-54C.

### Example 26. Secretion of Murine CD40L

To generate genetically engineered bacteria which are capable of secreting CD40L, mCD40L1(47-260) and mCD40L2(122-260) constructs as shown in Table 89 were generated according to methods described herein. mCD40L1(47-260) and mCD40L2(122-260) correspond to extracellular portion of the full length mCD40L and soluble form of mCD4L, respectively.

TABLE 31
Strains for secretion of murine CD40L

| Strain | | |
|---|---|---|
| Number | Genotype | Construct |
| SYN1557 | Nissle ΔPAL::CmR | — |
| (parental | | |
| strain) | | |
| SYN3366 | Nissle ΔPAL::CmR | pUC-ptet-phoA-mCD40L1 |
| | | (47-260) -V5-HIS |
| SYN3367 | Nissle ΔPAL::CmR | pUC- ptet-phoA-mCD40L2 |
| | | (112-260) -V5-HIS |

E. coli Nissle comprising plasmid-based tet-inducible constructs comprising ptet-PhoA-CD40L1 (47-260) (SYN3366) and tet-PhoA-CD40L2 (112-260) (SYN3367) and the parental control strain SYN1557 were grown overnight in LB medium. Cultures were diluted 1:100 in LB and grown shaking (200 rpm) to an optical density of 0.8, at which time the cultures were cooled down to room temperature and anhydrous tetracycline (ATC) was added to cultures at a concentration of 100 ng/mL, to induce expression of mCD40L1 and mCD40L2.

After 18 hours of induction, cells were spun down, and supernatant was collected. To generate cell free medium, the clarified supernatant was further filtered through a 0.22 micron filter to remove any remaining bacteria and placed on ice.

Supernatants were then analyzed by western blot. Proteins from 25 μL supernatant were transferred onto PVDF membranes and mCD40-L1 and mCD40L-2 were detected with an HRP-conjugated anti-V5 antibody (Biolegend). Results are shown in FIG. 55. A single band was detected around 32 kDa and 24 kDa for mCD40L1 and mCD40L2, respectively.

To determine whether the mCD40L secreted by the genetically engineered E. coli Nissle captured in the clarified supernatant can functionally bind to mCD40 and/or is detected by an anti-mCD40L antibody, an ELISA assay was performed by coating the plate with mCD40 or anti-mCD40L antibody. Results are shown in Table 32 and indicate that mCD40L1 (47-260) and mCD40L2 (112-260) secreted by the genetically engineered bacteria and can bind to mCD40.

TABLE 32
CD40 ELISA Binding Assay

| | Coating Materials | | | |
|---|---|---|---|---|
| Samples | mCD40 | Anti-mCD40L | IgG | PBS |
| PBS | 0.051 | 0.063 | 0.049 | 0.047 |
| Control | 0.054 | 0.065 | 0.052 | 0.054 |
| Secreted CD40-L1 | 0.231 | 0.394 | 0.052 | 0.049 |
| Secreted CD40-L2 | 0.639 | 0.825 | 0.052 | 0.05 |

### Example 27. Secretion of SIRPα and Variants and Anti-CD47 scFv

To generate genetically engineered bacteria which are capable of secreting anti-SIRPA, constructs shown in Table 33 were generated according to methods described herein. Sequences are shown include SEQ ID NOs: 1094-1104 and SEQ ID NOs: 1105-1121.

TABLE 33
Strains for secretion of SIRPα, SIRPα
variants and mCD47 scFv Ligand

| Strain | | |
|---|---|---|
| Number | Genotype | Construct |
| SYN1557 | Nissle ΔPAL::CmR | — |
| (parental | | |
| strain) | | |
| SYN2996 | Nissle ΔPAL::CmR | p15A-ptet-PhoA-FLAG-mSIRPa(32- |
| | | 373)-V5-HIS |
| SYN3159 | Nissle ΔPAL::CmR | pUC-ptet-PhoA-FLAG-CV1sirpα- |
| | | V5-HIS |
| SYN3160 | Nissle ΔPAL::CmR | pUC-ptet-PhoA-FLAG-FD6x2sirpα- |
| | | V5-HIS |
| SYN3021 | Nissle ΔPAL::CmR | pUC-ptet-PhoA-SIRPαCV1hIgG4- |
| | | V5-HIS |
| SYN3020 | Nissle ΔPAL::CmR | pUC-ptet-PhoA-FD6sirpαhIgG4- |
| | | V5-HIS |
| SYN3161 | Nissle ΔPAL::CmR | pUC-tet-PhoA-αmCD47scFv-V5-HIS |

E. coli Nissle strains SYN1557, SYN2996, SYN3159, SYN3160, SYN3021, SYN3020, and SYN3161, were grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression of SIRPα or SIRPα variants, or CD47 scFvs.

After 18 hours of induction, cells were spun down, and supernatants were collected. To generate cell free medium, the clarified supernatants were further filtered through a 0.22 micron filter to remove any remaining bacteria and placed on ice.

Supernatant was then analyzed by western blot. Proteins in 25 pt supernatant were transferred onto PVDF membranes and SIRPα and SIRPα variants and anti-CD47 scFv were detected using anti-V5-HRP antibody (Biolegend). Results are shown in FIG. 67. A single band was detected around 46 kDa for WT mSIRPα, 20 kDa for CV1SIRPα, 33 kDa for FD6×2 SIRPα, 42 kDa for FD6SIRPα-IgG4, 42 kDa for CV1SIRPα-IgG4, and 30 kDa for anti-CD47 scFv, respectively.

To determine whether the wild type SIRPα, SIRPα variants, and anti-CD47-scFvs secreted by the genetically engineered E. coli Nissle can functionally bind to CD47 and/or are detected by an anti-SIRPα antibody, an ELISA assay was performed by coating the plate with corresponding antibodies or ligands. Results are shown in Table 34 and indicate that both the secreted mSIRPα and anti-mCD47scFv by the genetically engineered bacteria can bind to mCD47.

TABLE 34
SIRPα/CD47 ELISA Binding Assay

Coating Materials

| Samples | mCD47 | Anti-mSIRPα | IgG | PBS |
|---|---|---|---|---|
| PBS | 0.051 | 0.054 | 0.054 | 0.051 |
| Control | 0.056 | 0.052 | 0.053 | 0.054 |
| Secreted WT | 1.069 | 0.829 | 0.056 | 0.059 |
| mSIRPα | | | | |

| | Coating Materials | | |
|---|---|---|---|
| Samples | mCD47 | IgG | PBS |
| PBS | 0.046 | 0.053 | 0.048 |
| Control | 0.049 | 0.051 | 0.048 |
| Secreted | 0.527 | 0.067 | 0.053 |
| anti-mCD47 scFv | | | |

To determine whether the SIRPα, SIRPα variants, and anti-CD47 scFv secreted from in E. coli Nissle bind to CD47 on mouse cells, flow cytometric analysis was performed using CT26 cells. CT26 is a mouse colon carcinoma cell line which expresses CD47 on its cell surface.

CT26 cells were grown in Dulbecco's Modified Eagle's Medium (DMEM) with 10% FBS and 1% Penicillin-Streptomycin. Cells were spun down, supernatant was aspirated, pellet was resuspended in 1 nil D-PBS, transferred into chilled assay tubes (1×10\hat{ }6 cells), and washed 3 times in D-PBS. Cells were resuspended in D-PBS with 0.5% BSA, to which the 10 μL of the supernatant (10× concentrated) were added and incubated for 1 hour at 4 C. Supernatant from SYN1557 was used as a baseline negative control. Cells were then resuspended in 0.5 ml PBS, diluted to proper concentration and analyzed on a flow cytometer. Results are shown in (FIG. 68 and FIG. 69). A population shift relative to baseline is observed for the samples containing the secreted SIRPα, SIRPα variants, and anti-CD47 scFv, with the greatest shift observed with SYN3021, expressing CV1SIRPα-IgG4.

Next, a competition assay was conducted to determine whether the murine SIRPα secreted by the genetically engineered bacteria could compete with the binding of a recombinant mSIRPα or an anti-CD47 antibody to murine CD47 on CT26 cells. CT26 cells were grown and flow cytometry protocol was conducted essentially as described above except that recombinant SIRPα (Rndsystems) or an anti-CD47 antibody (Biolegend) was added during the incubation of the secreted FD6×2sirpα or FD6sirpαhIgG4. FIG. 70 and FIG. 71 show the results of the competition with recombinant SIRPα and anti-CD47 antibody, respectively. Both recombinant SIRPα and anti-CD47 antibody were able to compete with the secreted SIRPα for the binding to CD47 on the CT26 cells.

### Example 28. Secretion of Hyaluronidase

To generate genetically engineered bacteria which are capable of secreting hyaluronidase, constructs were generated according to methods described herein (SYN2998: Nissle ΔPAL::CmR; p15A-ptet-RBS-PhoA--FLAG-human hyaluronidase-V5-His tags; SYN2997: Nissle ΔPAL::CmR; p15A-ptet-RBS-PhoA-FLAG-human hyaluronidase-V5-His; SYN3369: Nissle ΔPAL::CmR; p15A-ptet-RBS-PhoA-FLAG-leech hyaluronidase-V5-His).

E. coli Nissle strains SYN1557, SYN2997 (secreting mouse hyaluronidase), SYN2998 (secreting human hyaluronidase) and SYN3369 (secreting leech hyaluronidase), were grown overnight in LB medium. Cultures were diluted 1:100 in LB and grown shaking (200 rpm) to an optical density of 0.8 at which time culture was cooled down to room temperature and anhydrous tetracycline (ATC) was added to cultures at a concentration of 100 ng/mL to induce expression hyaluronidase.

After 18 hours of induction, cells were spun down, and supernatant was collected. To generate cell free medium, the clarified supernatant was further filtered through a 0.22 micron filter to remove any remaining bacteria and placed on ice.

Supernatants were then analyzed by western blot. Proteins in 25 μL supernatant were transferred onto PVDF membranes and hyaluronidase was detected using anti-V5-HRP antibody (Biolegend). Results are shown in FIG. 72. A single band was detected around 50 kDa for both secreted mouse and human hyaluronidases and around 57 kDa for leech hyaluronidase.

To determine whether the hyaluronidase secreted by the genetically engineered E. coli Nissle is active, a hyaluronidase activity assay on ability to cleave hyaluronan was performed, using biotinylated hyaluronate coated plate, according to manufacturer's instructions. Briefly, hyaluronidase will cleaved the polymer and result in the loss of biotin on plate which can then be detected by streptavidin-HPR and substrate. Results are shown in FIGS. 74A-74B and indicate that hyaluronidase secreted by the genetically engineered bacteria is able to degrade hyaluronan. The parental strain SYN1557 was used as a negative control. Results obtained for secreted leech hyaluronidase are shown in FIGS. 74A-74B.

### Example 29. An Engineered IL-15-Producing E. coli Nissle Strain

### Strain Construction and Biochemical Analysis

To generate an engineered E. coli Nissle strain capable of secreting biologically active interleukin 15 (IL-15) a fusion protein was constructed, in which IL-15 is fused to the minimal region of IL-15Rα required for the formation of a functional receptor, known as the Sushi domain. IL-15 and IL-15Rα form functional complexes, which stimulate cell signaling, and activation and proliferation of neighboring lymphocytes expressing IL-2Rβ and γc in a process called trans-presentation (see, e.g., Ochoa et al., High-density lipoproteins delivering interleukin-15; Oncoimmunology. 2013 Apr. 1; 2(4): e23410). The biological activity of IL-15 is greatly improved by two different modifications: an asparagine to aspartic acid substitution at amino acid 72 of IL-15 (Zhu X, Marcus WD, Xu W, Lee H I, Han K, Egan J O, Yovandich J L, Rhode P R, Wong H C. Novel human interleukin-15 agonists. J Immunol 2009; 183:3598-3607) and/or direct fusion with the sushi domain of IL-15Rα by mimicking trans-presentation of IL-15 by cell-associated IL-15Rα (Mortier et al., Soluble interleukin-15 receptor alpha (IL-15R alpha)-sushi as a selective and potent agonist of IL-15 action through IL-15R betagamma. Hyperagonist IL-15×IL-15R alpha fusion proteins. J Biol Chem 2006; 281:1612-9).

To produce the modified recombinant IL-15-Sushi fusion protein, IL-15 monomer with an N72D mutation was fused to the C-terminus of the sushi domain (78 amino acids) linked by a 20 amino acid linker. To the N-termini of the IL-15R sushi domain, a FLAG-tag and Factor Xa cleavage site was included to facilitate detection, purification and removal of the tag. To promote translocation to the periplasm, the IL15 fusion protein was cloned into a 10-member plasmid library. The coding sequence for the secreted fusion protein was codon optimized for expression in E. coli and ordered from IDT Technologies as a double-stranded DNA fragment. Once the DNA was received it was digested and ligated into the 10-member plasmid library using standard cloning procedures. Each member of the plasmid library contains a low-copy plasmid backbone and a Ptet promoter that drives expression of variable optimized ribosome-binding sites and secretion tags. Once the IL-15 fusion was cloned C-terminally of the secretion tags, the plasmids were transformed into SYN1557 (ΔPAL, diffusible outer membrane (DOM) phenotype) to create the IL-15-Sushi secretion strains SYN3516-SYN3525. Non-limiting examples of construct sequences include SEQ ID NOs: 1132-1137 and SEQ ID NOs: 1138-1144. Table 35 provides a description of the IL-15-Sushi strains. Table 36 provides a listing of strains generated using WT IL-15.

TABLE 35
Strain descriptions

| ID | Genotype | Construct |
|---|---|---|
| SYN3424 | PAL (PAL::Cm) | pBR322.Ptet.PpiA |
| | | (ECOLIN_18620)-IL-15 |
| SYN3423 | PAL (PAL::Cm) | pBR322.Ptet.phoA-IL-15 |
| SYN3422 | PAL (PAL::Cm) | pBR322.Ptet.PelB-IL-15 |
| SYN3421 | PAL (PAL::Cm) | pBR322.Ptet.OppA- IL-15 |
| SYN3420 | PAL (PAL::Cm) | pBR322.Ptet.MalE-IL-15 |
| SYN3419 | PAL (PAL::Cm) | pBR322.Ptet.HdeB-IL-15 |
| SYN3418 | PAL (PAL::Cm) | pBR322.Ptet.GspD-IL-15 |
| SYN3417 | PAL (PAL::Cm) | pBR322.Ptet.Gltl- IL-15 |
| SYN3416 | PAL (PAL::Cm) | pBR322.Ptet.DsbA- IL-15 |
| SYN3415 | PAL (PAL::Cm) | pBR322.Ptet.Adhesin- IL-15 |

TABLE 36
Strain descriptions

| ID | Genotype | Construct |
|---|---|---|
| SYN3525 | | pBR322.Ptet PpiA |
| | | (ECOLIN_18620)-IL-15-Sushi |
| SYN3524 | PAL (PAL::Cm) | pBR322.Ptet.phoA-IL-15-Sushi |
| SYN3523 | PAL (PAL::Cm) | pBR322.Ptet.PelB-IL-15-Sushi |
| SYN3522 | PAL (PAL::Cm) | pBR322.Ptet.OppA-IL-15-Sushi |
| SYN3521 | PAL (PAL::Cm) | pBR322.Ptet.MalE-IL-15-Sushi |
| SYN3520 | PAL (PAL::Cm) | pBR322.Ptet.HdeB-IL-15-Sushi |
| SYN3519 | PAL (PAL::Cm) | pBR322.Ptet.GspD-IL-15-Sushi |
| SYN3518 | PAL (PAL::Cm) | pBR322.Ptet.Gltl- IL-15-Sushi |
| SYN3517 | PAL (PAL::Cm) | pBR322.Ptet.DsbA- IL-15-Sushi |
| SYN3516 | PAL (PAL::Cm) | pBR322.Ptet.Adhesin- IL-15-Sushi |

Production of and in vitro quantification of IL-15 by SYN3525

To assay for production of IL-15 and/or to quantify bioactivity, cultures were grown and induced, then supernatants were harvested and quantified by ELISA. Prior to assay date, both the negative control strain (SYN1557) and the IL-15-sushi domain producing strains (SYN3516-SYN3525) were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ng/mL). These cultures were placed at 30° C. with shaking for 4 hours to induce IL-15-sushi fusion protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

To evaluate the production of IL-15 fusions by the plasmid library, cultures of SYN1557 and the IL-15—producing library were induced and harvested in duplicate. These supernatants were serially-diluted and quantified using an IL-15 ELISA Kit (Human IL-15 Quantikine ELISA Kit—D1500, R&D Systems, Minneapolis, Minn.). The data from screening the secretion library displayed in Table 37 shows maximal production from SYN3525, which contains the PpiA secretion signal from E. coli as the leader peptide. SYN1557 supernatants showed no cross-reactivity in the ELISA (data not shown).

TABLE 37

| | Secretion |
|---|---|
| Strain | (ng/ml; average of 2) |
| SYN3525 | 84.78 |
| SYN3524 | 46.25 |
| SYN3523 | 92.88 |
| SYN3522 | 53.84 |
| SYN3521 | 54.71 |
| SYN3520 | 80.36 |
| SYN3519 | 37.98 |
| SYN3518 | 59 |
| SYN3517 | 42.81 |
| SYN3516 | 36.33 |

To further evaluate the production of IL-15 from SYN3525, the strain was grown and induced in baffled flasks to generate maximum yield. From the filtered supernatants of SYN3525, samples of SYN1557 and SYN3525 were diluted in triplicate and run on an ELISA Kit (Human IL-15 Quantikine ELISA Kit—D1500, R&D Systems, Minneapolis, Minn.). The results of these analyses are shown in Table 38. The results showed that under maximal induction conditions the SYN3525 supernatant contained between 733-795 ng/mL of material that reacted positively in the IL-15 ELISA assay. In contrast, the SYN1557 supernatants had undetectable levels (not shown).

TABLE 38
SYN3525 supernatant results from three different ELISA runs.

| SYN3525 | IL-15 (ng/ml) |
|---|---|
| Run 1 | 795.23 |
| Run 2 | 733.75 |
| Run 3 | 792.80 |
| AVERAGE | 773.93 |
| SEM (n=3) | 20.10 |

For comparison, Secretion from constructs expressing WT IL-15 are shown in Table 39.

TABLE 39

| Strain number | ng/mL (Average of 2) |
|---|---|
| SYN3424 | 2.26 |
| SYN3423 | 7.83 |
| SYN3422 | 1.78 |
| SYN3421 | 0.09 |
| SYN3420 | 0.04 |
| SYN3419 | 0.83 |
| SYN3418 | 10.42 |
| SYN3417 | 0.37 |
| SYN3416 | 1.06 |
| SYN3415 | 0.43 |

### Example 30. Functional Assay for Bacterially Secreted IL-15

Next a functional assay was conducted to demonstrate that IL-15 secreted from SYN3525 was functional. STAT3 and STATS have been shown to be phosphorylated by IL-15Ralpha upon ligand binding.

T-cells were purified from human leukopheresis via Miltenyi untouched pan-T-cell kit (yield=˜5e7 cells/prep). IL15Rα expression was induced via mild stimulation with Phytohaemagglutinin P (PHA) for 48 hrs. The activated T-cells were treated with supernatants from IL15 expressing bacteria SYN 3525 for 15 min. The treated cells were fixed in paraformaldehyde based solution followed by harsh permeabilization in 90% methanol. Modulation of phospho-STATS was quantified by multi-color flow cytometry in IL15R+CD3+ cells. Results are shown in FIG. 46 and show that SYN3525 derived IL15 exhibits bioactivity comparable to that of rhIL15. Consistent results were observed between supernatants from two individually bacterial preps.

### Example 31. Construction of a Dimerized IL-12 for Secretion

Strain Engineering:

To generate an engineered E. coli Nissle strain capable of secreting biologically active Interleukin 12 (IL-12) heterodimer, a construct was generated in which two interleukin-12 monomer subunits (IL-12A (p35) and IL-12B (p40)) were covalently linked by a linker.

To facilitate the dimerization of recombinant human IL-12 protein produced from E. coli Nissle, a 15 amino acid linker of ‘GGGGSGGGGSGGGGS’ (SEQ ID NO: 1247) was inserted between two monomer subunits (IL-12A (p35) and IL-12B (p40) to produce a forced dimer human IL-12 (diIL-12) fusion protein, and the sequence was codon-optimized.

To promote translocation to the periplasm, secretion tags were added to the N-terminus of the diIL-12 fusion protein. The DNA sequence containing the elements of the inducible Ptet promoter, ribosome binding site, diIL-12 coding sequence and other necessary linkers were synthesized by IDT Technologies and subsequently cloned into a high copy number plasmid vector. The plasmid was transformed into SYN1555, SYN1556, SYN1557 or SYN1625 (comprising the nlpl, tolA, PAL, or 1pp deletion respectively to create the diffusible membrane phenotype) to create the dimerized human IL-12 (diIL-12) secretion strains.

TABLE 40
Non-limiting IL-12 Construct Sequences

| Construct comprising secretion tag 19410 - human | SEQ ID NO: |
|---|---|
| IL-12 (p35) - Linker (15aa) - human IL-12 (p40) | 1235 |
| Construct comprising secretion tag dsba - human | SEQ ID NO: |
| IL-12 (p35) - Linker (15aa) - human IL-12 (p40) | 1146 |
| Construct comprising secretion tag phoA - human | SEQ ID NO: |
| IL-12 (p35) - Linker (15aa) - human IL-12 (p40) | 1147 |
| Construct comprising secretion tag tolB - human | SEQ ID NO: |
| IL-12 (p35) - Linker (15aa) - human IL-12 (p40) | 1148 |
| Construct comprising secretion tag malE - human | SEQ ID NO: |
| IL-12 (p35) - Linker (15aa) - human IL-12 (p40) | 1149 |
| Construct comprising secretion tag mglB - human | SEQ ID NO: |
| IL-12 (p35) - Linker (15aa) - human IL-12 (p40) | 1150 |
| Construct comprising secretion tag ompF - human | SEQ ID NO: |
| IL-12 (p35) - Linker (15aa) - human IL-12 (p40) | 1151 |
| Construct comprising secretion tag ompA - human | SEQ ID NO: |
| IL-12 (p35) - Linker (15aa) - human IL-12 (p40) | 1152 |
| Construct comprising secretion tag tort - human | SEQ ID NO: |
| IL-12 (p35) - Linker (15aa) - human IL-12 (p40) | 1153 |
| Construct comprising secretion tag lamB - human | SEQ ID NO: |
| IL-12 (p35) - Linker (15aa) - human IL-12 (p40) | 1154 |
| Construct comprising secretion tag pelB - human | SEQ ID NO: |
| IL-12 (p35) - Linker (15aa) - human IL-12 (p40) | 1156 |
| human IL-12 (p35) - Linker (15aa) - human IL-12 (p40) | SEQ ID NO: |
| | 1168 |

TABLE 41
Non-limiting IL-12 Construct Polypeptide Sequences

Description

| Construct comprising secretion tag 19410 - human | SEQ ID NO: |
|---|---|
| IL-12 (p35) - Linker (15aa) - human IL-12 (p40); | 1169 |
| (includes C terminal V5 and HIS tag and FLAG tag | |
| located at C terminus of secretion tag) | |
| Construct comprising secretion tag dsba - human | SEQ ID NO: |
| IL-12 (p35) - Linker (15aa) - human IL-12 (p40); | 1170 |
| (includes C terminal V5 and HIS tag and FLAG tag | |
| located at C terminus of secretion tag) | |
| Construct comprising secretion tag phoA - human | SEQ ID NO: |
| IL-12 (p35) - Linker (15aa) - human IL-12 (p40); | 1171 |
| (includes C terminal V5 and HIS tag and FLAG tag | |
| located at C terminus of secretion tag) | |
| Construct comprising secretion tag tolB - human | SEQ ID NO: |
| IL-12 (p35) - Linker (15aa) - human IL-12 (p40); | 1172 |
| (includes C terminal V5 and HIS tag and FLAG tag | |
| located at C terminus of secretion tag) | |
| Construct comprising secretion tag malE - human | SEQ ID NO: |
| IL-12 (p35) - Linker (15aa) - human IL-12 (p40); | 1173 |
| (includes C terminal V5 and HIS tag and FLAG tag | |
| located at C terminus of secretion tag) | |
| Construct comprising secretion tag mglB - human | SEQ ID NO: |
| IL-12 (p35) - Linker (15aa) - human IL-12 (p40); | 1174 |
| (includes C terminal V5 and HIS tag and FLAG tag | |
| located at C terminus of secretion tag) | |
| Construct comprising secretion tag ompF - human | SEQ ID NO: |
| IL-12 (p35) - Linker (15aa) - human IL-12 (p40); | 1175 |
| (includes C terminal V5 and HIS tag and FLAG tag | |
| located at C terminus of secretion tag) | |
| Construct comprising secretion tag ompA - human | SEQ ID NO: |
| IL-12 (p35) - Linker (15aa) - human IL-12 (p40); | 1176 |
| (includes C terminal V5 and HIS tag and FLAG tag | |
| located at C terminus of secretion tag) | |
| Construct comprising secretion tag tort - human | SEQ ID NO: |
| IL-12 (p35) - Linker (15aa) - human IL-12 (p40); | 1177 |
| (includes C terminal V5 and HIS tag and FLAG tag | |
| located at C terminus of secretion tag) | |
| Construct comprising secretion tag lamB - human | SEQ ID NO: |
| IL-12 (p35) - Linker (15aa) - human IL-12 (p40); | 1178 |
| (includes C terminal V5 and HIS tag and FLAG tag | |
| located at C terminus of secretion tag) | |
| Construct comprising secretion tag pelB - human | SEQ ID NO: |
| IL-12 (p35) - Linker (15aa) - human IL-12 (p40); | 1179 |
| (includes C terminal V5 and HIS tag and FLAG tag | |
| located at C terminus of secretion tag) | |
| human IL-12 (p35) - Linker (15aa) - human IL-12 (p40) | SEQ ID NO: |
| | 1191 |
| Human IL-12 monomer p35 | SEQ ID NO: |
| | 1192 |
| Human IL-12 monomer p40 | SEQ ID NO: |
| | 1193 |
| Linker | SEQ ID NO: |
| | 1194 |

Production of diIL-12 by SYN3466 to SYN3505 for In Vitro Assays:

To assay for production of IL-12 and/or to quantify bioactivity, cultures were grown and induced, then supernatants were harvested and quantified via ELISA, as follows using shaker plate. Both the negative control strain (SYN1555, SYN1556, SYN1557 or SYN1625) and the diIL-12-producing strain (SYN3466 to SYN3505) were struck onto LB agar plates and grown at 37° C. with chloramphenicol, or chloramphenicol and carbenicillin, respectively. After overnight growth, an individual colony was picked and used to inoculate a 4 mL starter culture of 2YT broth for future shaker plate to be grown. The starter cultures were inoculated with the same antibiotics used in the agar plates. When the starter culture reached saturation, the culture was back-diluted 1:25 into a new shaker plate of 2YT with appropriate antibiotics. This starter culture was allowed to grow at 37° C. for 2 hours to an OD600=˜0.8-1. The culture was then induced by adding aTc inducer (100 microg/mL). These induced cultures were incubated at 30° C. with shaking for 4 hours to promote IL-12 production.

When the production phase was complete, the shaker plate of cultures was removed from the incubator and centrifuged at 20000×g for 10 minutes to pellet all cells. The supernatants were kept for ELISA analysis.

Quantification of diIL-12 in supernatants by ELISA:

To evaluate the amounts of diIL-12 in the supernatants, samples were assayed using a Human IL-12 p70 Quantikine ELISA Kit from R&D systems. The results of these analyses are shown in Table 42.

The results showed that the supernatants contained between 17 and 309 pg/mL of material that reacted positively in the IL-12 ELISA assay. In contrast, the SYN1557 supernatants had undetectable levels.

TABLE 42
Supernatant results from ELISA analysis (pg/mL)

| Signal Peptides, | | | | | | |
|---|---|---|---|---|---|---|
| Host strain | Vector | 19410 | pelB | tort | LamB | OmpF |
| Nissle ΔnlpI::CmR | 15.1 | 130.7 | 61.4 | 255.6 | 81.6 | 17.0 |
| Nissle ΔtolA::CmR | 12.8 | 47.7 | 93.0 | 205.3 | 189.8 | 220.2 |
| Nissle | 10.2 | 142.3 | 31.2 | 266.7 | 167.0 | 62.8 |
| ΔΔPAL::CmR | | | | | | |
| Nissle Δlpp::CmR | 10.5 | 233.7 | 57.9 | 278.6 | 107.2 | 128.4 |

| Signal Peptides, | | | | | | |
|---|---|---|---|---|---|---|
| Host strain | OmpF | mglB | malE | tolB | phoA | dsbA |
| Nissle ΔnlpI::CmR | 17.0 | 194.4 | 224.7 | 234.2 | 241.9 | 99.3 |
| Nissle ΔtolA::CmR | 220.2 | 227.0 | 194.4 | 187.0 | 309.1 | 146.0 |
| Nissle ΔPAL::CmR | 62.8 | 218.4 | 256.5 | 250.0 | 203.7 | 237.0 |
| Nissle Δlpp::CmR | 128.4 | 203.3 | 242.3 | 197.2 | 250.7 | 101.9 |

### Example 32. An Engineered IL-15-Producing E. coli Nissle Strain

Strain Engineering

To generate an engineered E. coli Nissle strain capable of secreting biologically active interleukin 15 (IL-15) a fusion protein was constructed, in which IL-15 is fused to the minimal region of IL-15Rα required for the formation of a functional receptor, which is known as Sushi domain. IL-15 and IL-15Rα form functional complexes, which stimulate cell signaling, and activation and proliferation of neighboring lymphocytes expressing IL-2Rβ and γc in a process called trans-presentation (see, e.g., Ochoa et al., High-density lipoproteins delivering interleukin-15; Oncoimmunology. 2013 Apr. 1; 2(4): e23410). The biological activity of IL-15 is greatly improved by direct fusion with the sushi domain of IL-15Rα by mimicking trans-presentation of IL-15 by cell-associated IL-15Rα (Mortier et al., Soluble interleukin-15 receptor alpha (IL-15R alpha)-sushi as a selective and potent agonist of IL-15 action through IL-15R betagamma. Hyperagonist IL-15×IL-15R alpha fusion proteins. J Biol Chem 2006; 281:1612-9).

To produce the recombinant IL-15-Sushi fusion protein, IL-15 monomer was fused to the C-terminus of the sushi domain linked by a 20 amino acid linker. To promote translocation to the periplasm, 19410, tort, or pelB secretion tag was added to the N-terminus of IL-15-Sushi fusion protein. The DNA sequence containing the Ptet promoter, RBS, coding sequence for IL-15-Sushi and other necessary linkers were synthesized by IDT Technologies and subsequently cloned into a high copy number plasmid vector provided by IDT Technologies under the control of a tet-inducible promoter. The plasmids were transformed into SYN1557 (ΔPAL, diffusible outer membrane (DOM) phenotype) or SYN94 (no PAL deletion E. coli Nissle strain) to create the IL-15-Sushi secretion strains SYN3458 to SYN3463. Table 43 and Table 44 list a number of non-limiting examples of construct sequences.

TABLE 43
Non-limiting IL-15 Construct Polypeptide Sequences

| Description | Sequence |
|---|---|
| Human IL-15Rα sushi domain | |
| Construct comprising 19410 secretion tag- | SEQ ID NO: 1195 |
| Sushi- linker - human IL-15 | |
| Construct comprising tort secretion tag- | SEQ ID NO: 1196 |
| Sushi- linker - human IL-15 | |
| Construct comprising pelB secretion tag- | SEQ ID NO: 1197 |
| Sushi- linker - human IL-15 | |
| Sushi- linker - human IL-15 | SEO ID NO: 1198 |

TABLE 44
Non-limiting IL-15 Construct Polynucleotide Sequences

| Description | Sequence |
|---|---|
| Human IL-15Rα sushi domain | SEQ ID NO: 1345 |
| Construct comprising 19410 secretion tag- | SEQ ID NO: 1200 |
| Sushi- linker - human IL-15 | |
| Construct comprising tort secretion tag- | SEQ ID NO: 1201 |
| Sushi- linker - human IL-15 | |
| Construct comprising pelB secretion tag- | SEQ ID NO: 1202 |
| Sushi- linker - human IL-15 | |
| Sushi- linker - human IL-15 | SEQ ID NO: 1203 |
| Human IL-1 | SEQ ID NO: 1204 |
| Linker | SEQ ID NO: 1199 |

Production of IL-15 by SYN3458 to SYN3463 for in vitro Assays:

To assay for production of IL-15 and/or to quantify bioactivity, cultures were grown and induced, then supernatants were harvested and quantified by ELISA. Prior to assay date, both the negative control strain (SYN1557) and the IL-15-sushi domain producing strain SYN3458 to SYN3463 were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ug/mL). These cultures were placed at 30° C. with shaking for 4 hours to induce IL-15-sushi fusion protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

### Quantification of IL-15 in SYN3458 to SYN3463 Supernatants by ELISA:

To evaluate the production of IL-15 in the filtered supernatants, samples of SYN1557 and SYN3458 to SYN3463 were diluted in triplicate and run on an Human IL-15 Quantikine ELISA Kit (Ra&D Systems). The results of these analyses are shown in Table 45. The results showed that the SYN3458 to SYN3463 supernatant contained between 4 and 275 ng/mL of material that reacted positively in the IL-15 ELISA assay. In contrast, the SYN94 and SYN1557 supernatants had undetectable levels (not shown).

TABLE 45
Supernatant results from three different ELISA runs.

| final strain | host strain | plasmid | ng/mL |
|---|---|---|---|
| SYN3460 | SYN1557 | ptet-pelBss-hIL15-SUSHI-fusion | 275 |
| SYN3461 | SYN1557 | ptet-19410ss-hIL15-SUSHI-fusion | 166 |
| SYN3458 | SYN1557 | ptet-tortss-hIL15-SUSHI-fusion | 59 |
| SYN3459 | SYN94 | ptet-tortss-hIL15-SUSHI-fusion | 78 |
| SYN3462 | SYN94 | ptet-pelBss-hIL15-SUSHI-fusion | 4 |
| SYN3463 | SYN94 | ptet-19410ss-hIL15-SUSHI-fusion | 72 |

### Example 33. CXCL10 Secretion

To produce the recombinant CXCL10 chemokine, a synthetic construct was devised (no data provided). The secreted/soluble form of the CXCL10 protein was codon optimized for expression in E. coli and ordered from IDT Technologies as a double-stranded DNA fragment. To promote translocation to the periplasm, the CXCL10 soluble protein was cloned into a 10-member plasmid library. Once the DNA was received it was digested and ligated into the 10-member plasmid library using standard cloning procedures. Each member of the plasmid library contains a low-copy plasmid backbone and a Ptet promoter that drives expression of variable optimized ribosome-binding sites and secretion tags. Once the CXCL10 was cloned C-terminally of the secretion tags, the plasmids were transformed into SYN1557 (OPAL, diffusible outer membrane (DOM) phenotype) to create the CXCL10 secretion strains, SYN3404 and SYN3406-SYN3414. Construct sequences include SEQ ID NO: 1205, SEQ ID NO: 1206, SEQ ID NO: 1208, and SEQ ID NO: 1209.

TABLE 46
Strain Descriptions

| ID | Genotype | Construct |
|---|---|---|
| SYN3414 | PAL (PAL::Cm) | p15a.Ptet.PpiA-CXCL10 |
| SYN3413 | PAL (PAL::Cm) | p15a.Ptet.phoA-CXCL10 |
| SYN3412 | PAL (PAL::Cm) | p15a.Ptet.PelB-CXCL10 |
| SYN3411 | PAL (PAL::Cm) | p15a.Ptet.OppA-CXCL10 |
| SYN3410 | PAL (PAL::Cm) | p15a.Ptet.MalE-CXCL10 |
| SYN3409 | PAL (PAL::Cm) | p15a.Ptet.HdeB-CXCL10 |
| SYN3408 | PAL (PAL::Cm) | p15a.Ptet.GspD-CXCL10 |
| SYN3407 | PAL (PAL::Cm) | p15a.Ptet.Gltl-CXCL10 |
| SYN3406 | PAL (PAL::Cm) | p15a.Ptet.DsbA-CXCL10 |
| SYN3404 | PAL (PAL::Cm) | p15a.Ptet.Adhesin-CXCL10 |

To assay for production of CXCL10 and/or to quantify bioactivity, cultures were grown and induced, then supernatants were harvested and quantified by ELISA. Prior to assay date, both the negative control strain (SYN1557) and the CXCL10-producing strains (SYN3404-SYN3414) were struck onto LB agar plates and grown at 37° C. with the appropriate antibiotics. A 2 mL starter culture of 2YT broth was inoculated with a single colony for every 50 mL of induced culture to be grown, allowed to grow at 37° C. for 2 hours, spun down at 8000×g for 10 minutes, and cells were resuspended in 2YT media of the original volume with the anhydrotetracycline (aTc) inducer (100 ng/mL). These cultures were placed at 30° C. with shaking for 4 hours to induce CXCL10 protein production. Next, cultures were removed from the incubator and centrifuged at 20,000×g for 10 minutes to pellet all cells and the supernatants were passed through a 0.22 μm filter to yield sterilized supernatants. These supernatants were used in cell-based assays.

To evaluate the production of CXCL10 by the plasmid library, cultures of SYN1557 and the CXCL10—producing library were induced and harvested in duplicate. These supernatants were serially-diluted and quantified using a CXCL10 ELISA Kit (Human CXCL10/IP-10 Quantikine ELISA Kit—DIP100, R&D Systems, Minneapolis, Minn.). The data from screening our secretion library displayed in Table 47 shows maximal production from SYN3413, which contains the PhoA secretion signal from E. coli as the leader peptide. SYN1557 supernatants showed no cross-reactivity in the ELISA (data not shown).

TABLE 47
CXCL10 Secretion

| Strain | |
|---|---|
| Name | ng/mL (Average of 2) |
| SYN3414 | 5.54 |
| SYN3413 | 52.41 |
| SYN3412 | 11.06 |
| SYN3411 | 0.14 |
| SYN3410 | 0.09 |
| SYN3409 | 3.66 |
| SYN3408 | 35.79 |
| SYN3407 | 1.25 |
| SYN3406 | 1.22 |
| SYN3405 | 1.07 |

To further evaluate the production of CXCL10 from SYN3413, the strain was grown and induced in baffled flasks to generate maximum yield. From the filtered supernatants of SYN3413, samples of SYN1557 and SYN3413 were diluted in triplicate and run on an ELISA Kit (Human CXCL10/IP-10 Quantikine ELISA Kit—DIP100, R&D Systems, Minneapolis, Minn.). The results of these analyses are shown in Table 112. The results showed that under maximal induction conditions the SYN3413 supernatant contained between 199-232 ng/mL of material that reacted positively in the CXCL10 ELISA assay. In contrast, the SYN1557 supernatants had undetectable levels (not shown).

TABLE 48
Concentration of Secreted hCXCL10 from triplicate SYN3414

| SYN3414 | CXCL10 (ng/ml) |
|---|---|
| Run 1 | 199.71 |
| Run 2 | 231.96 |
| Run 3 | 232.16 |
| AVERAGE | 221.28 |
| SEM (n = 3) | 10.78 |

### Functional Assay for CXCL10

To determine whether CXCL10 secreted from any of the strains described above is functional, a Chemotaxis Assay is performed, essentially as described in Mikucki et al. (Mikucki, et al., Non-redundant Requirement for CXCR3 Signaling during Tumoricidal T Cell Trafficking across Tumor Vascular Checkpoints; Nat Commun. 2015; 6: 7458, the contents of which is herein incorporated by reference in its entirety).

Briefly, cell trace labeled naïve (or expanded) human CD8+ T cells are transferred into 24-well transwell plate (5 uM pore) with T cells on top (5×105 cells) and rCXCL10/supernatants on bottom, in the presence or absence of PTX or anti-CXCL10)->Cells are incubated for 3 hrs, and migrated cells are counted by flow cytometry. Alternatively, phosphoAKT is measured by flow, western or ELISA.

### Example 34. Generation of STING Agonist Producing Strains

To generate STING agonist strains, DacA (Listeria monocytogenes cyclic di AMP synthase) was cloned into p15 under the control of the Pₜₑₜpromoter; to generate the strain as described in Table 50. Summarizes sequences of the constructs.

TABLE 49
c-di-AMP producing strain

| Strain: | Genotype |
|---|---|
| SYN3527 | Nissle p15A Ptet-DacA (listeria |
| | monocytogenes cyclic di AMP |
| | synthase) |

### Example 35. In Vitro STING Agonist Production

The ability of the newly generated strain to produce c-di-AMP was first assessed in vitro.

E. coli Nissle strains SYN3527 (comprising the DacA construct) and a control strain were grown overnight in LB medium. Cultures were diluted 1:25 in M9 minimal media supplemented with 0.5% glucose (w/v) and grown shaking (350 rpm) at 37° C. for 2 hours. Cultures were diluted to an optical density of 0.5 at which time anhydrous tetracycline (ATC) was added to cultures at a concentration of 100 ng/mL to induce expression of DacA. After 4 hours of induction, samples were removed for LC/MS analysis of cyclic dinucleotide production. Samples were centrifuged for 20 minutes at 5000 RPM to separate cellular and extracellular fractions. Cell pellets were then used to determine intracellular cyclic-di-AMP and the media supernatants were used to determined extracellular accumulation of cyclic-di-AMP. Concentrations were determined via LC/MS.

Results are shown in FIG. 2. and indicate that engineered strains were able to produce c-di-AMP intracellularly and, to a lesser extent, extracellularly. For the wild-type control, cyclic-di-AMP was not found in either the intracellular or extracellular fractions (data not shown).

FIG. 3 depicts a follow on study conducted essentially as described above.

### Example 36. Bacterially Produced STING Agonists Induce the Immune Response

To determine whether bacterially produced STING agonists or the bacterial chassi themselves is responsible for induction of the immune response, live and heat-killed SYN3527 (comprising plasmid based DacA under control of a tetracycline-inducible promoter (p15-ptet-DacA)); were added to the supernatant of RAW 267.4 mouse macrophage cell line and levels of IFN-beta1 mRNA were measured.

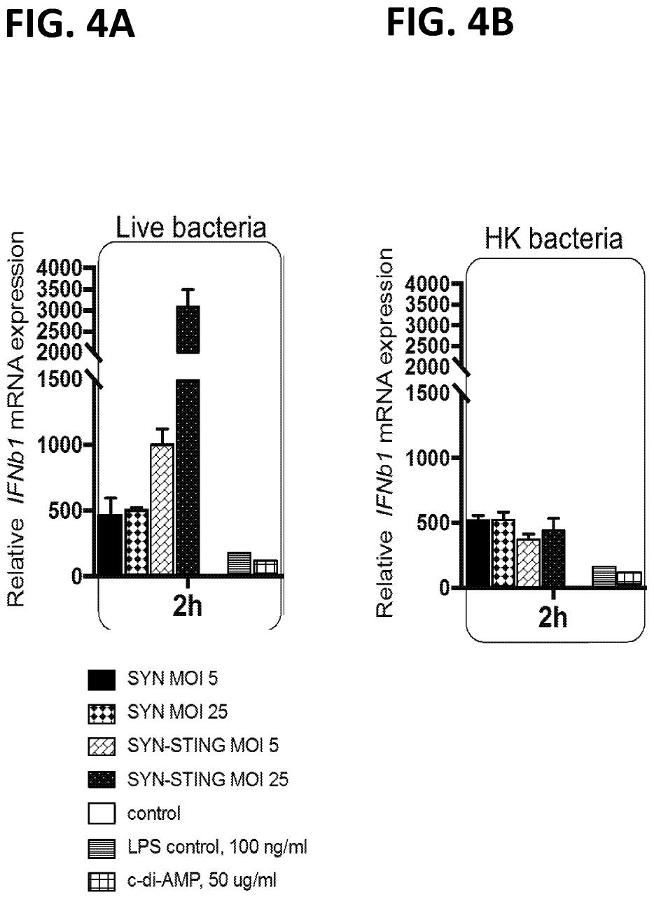

As seen in FIG. 4, IFNb1 mRNA expression increases with c-di-AMP secreting strain dose-dependently, but not when heat killed.

### Example 37. Activity of STING Agonist Producing Strain In Vivo

To determine the tolerability, colonization and activity of the STING agonist producing strain SYN3527 (E. coli Nissle comprising plasmid-based, tetracycline-inducible p15a Ptet-DacA from Listeria monocytogenes, tumor volume, weight and T cell response was assessed in the B16 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

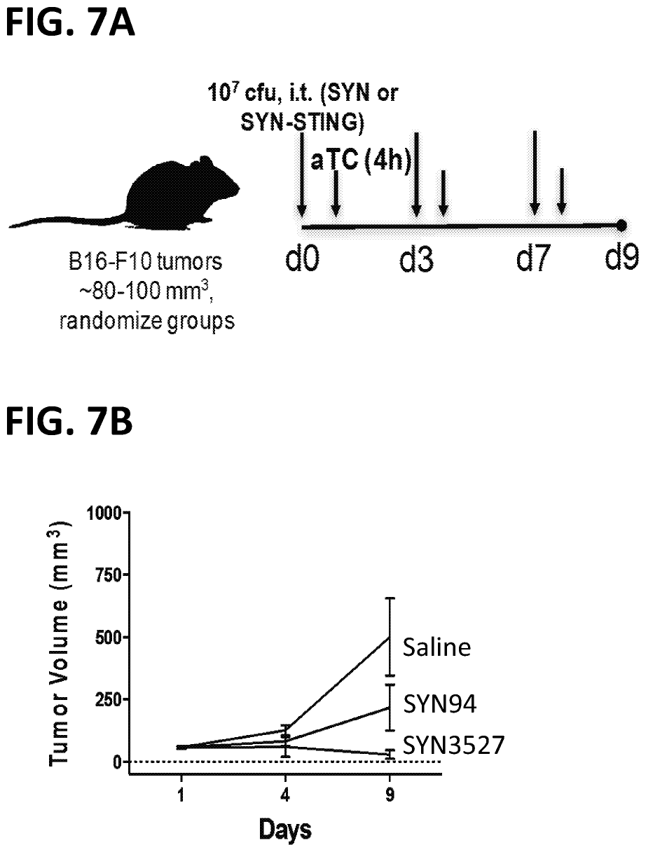

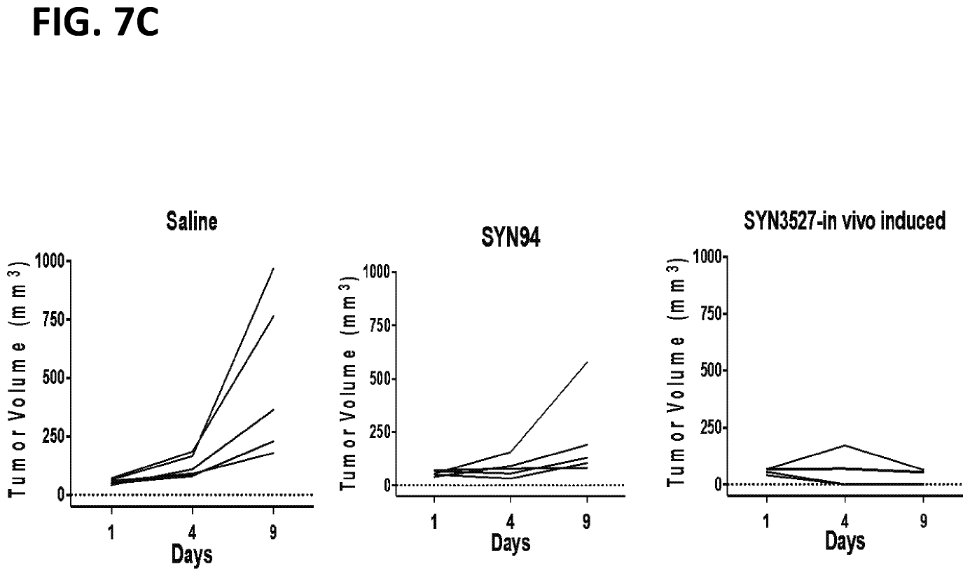

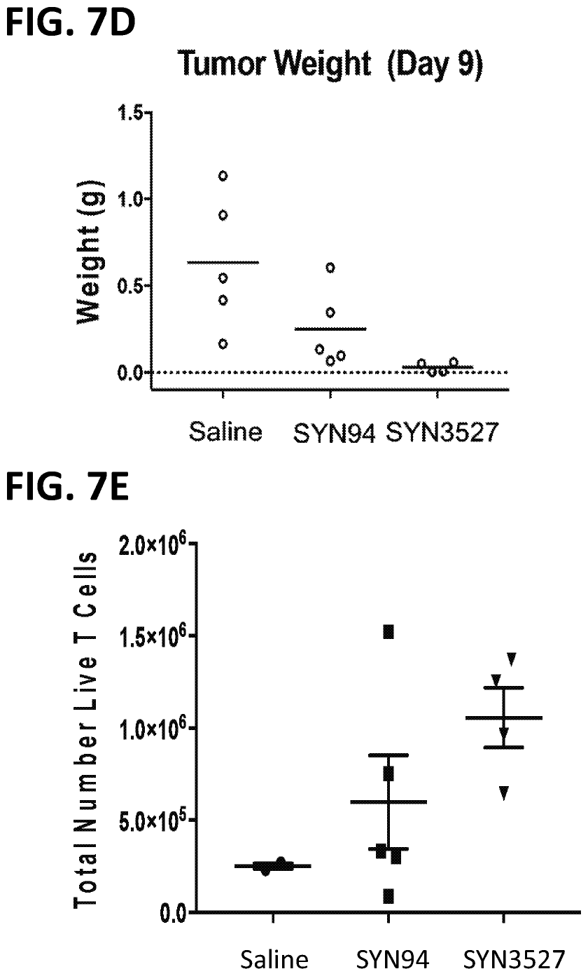

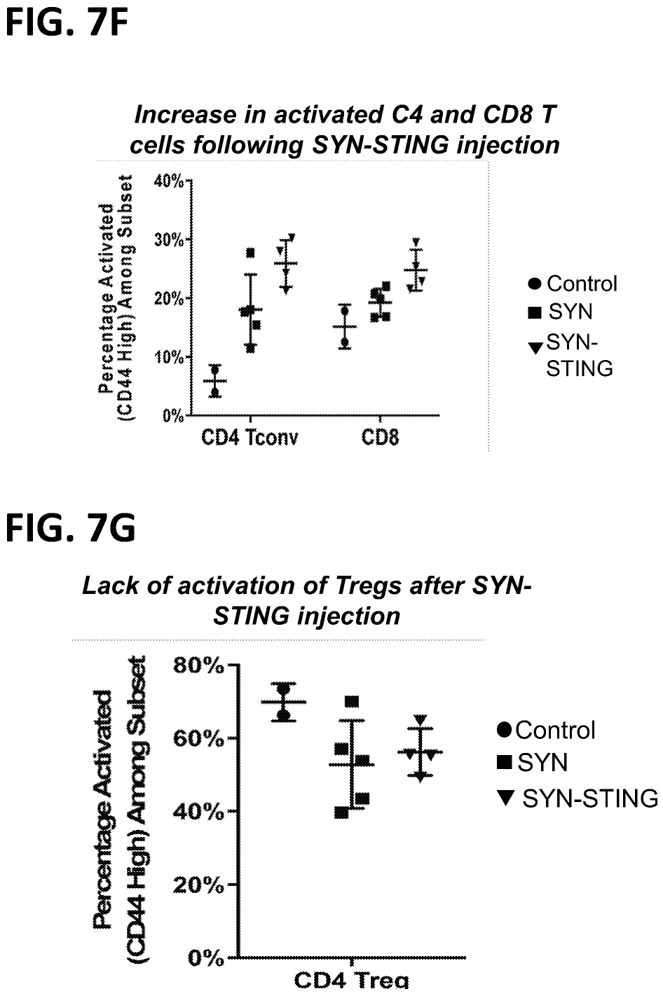

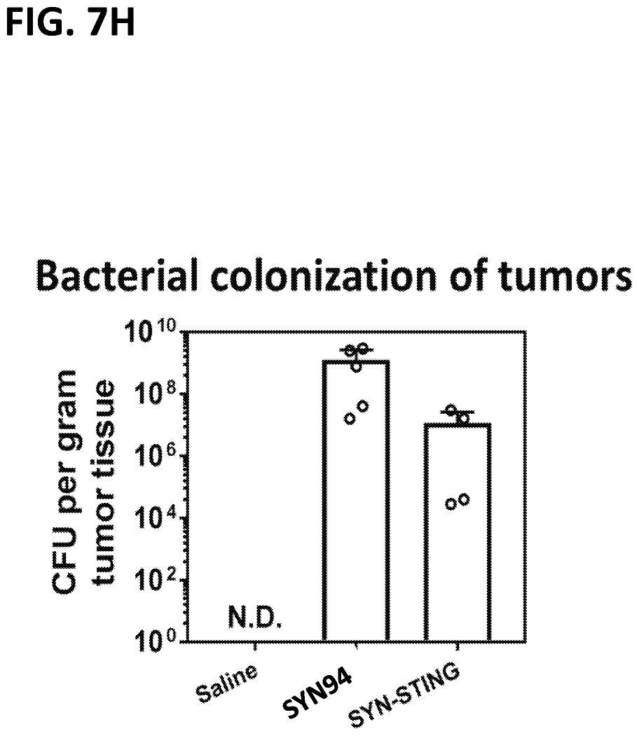

Mice were implanted with B16 tumors, and mice injected intratumorally with bacteria producing STING agonists and controls, according to the time line described below and in FIG. 7A. Samples taken at various timepoints were processed for CFU counts per gram of tumor, cytokine analysis (IFN-beta and T cell panels) and flow cytometric analysis of tumor draining lymph nodes (TDLN).

Briefly, B16 cells were implanted (2×10e5 cells/mouse in PBS) SC into the right flank of each animal on day −9. On day Day −3 tumor growth was monitored; when the tumors reached ˜50-80 mm\hat{ }3 on day 1, mice were randomized into groups (N=15 per group/5 mice/timepoint) for intratumor dosing as follows: saline (control, group 1), SYN94 (streptomycin resistant Nissle, group 2, 1×10e7), and SYN3527 (STING Agonist, group 3, 1×10e7) Animals were dosed with appropriate bacteria based on group or saline (to control for injection). Four hours later, mice were treated with 10 ug ATC (anhydrotetracycline) via intraperitoneal injection. On day 2, 5 mice from each treatment group were sacrificed 5 mice/group (Group 1-4) 24 hours after ATC dose and samples were processed for analysis. On day 4, mice were weighed, tumor volumes were measured, and mice were dosed with the appropriate treatment/group. Four hours later, mice were injected with 10 ug ATC. On day 5, 5 mice/group were sacrificed 24 hours after the ATC dose. On day 8, the tumors in the remaining mice were weighed, measured and mice were dosed with appropriate treatment/group. On day 9, 5 mice/group were sacrificed for 24 hours after ATC dose.

Tumor volume at day 1, 4, and 9 are shown in FIG. 7B and for individual mice in FIG. 7C and indicate that administration of SYN3527 results in rejection or control of tumor growth over this time period (p<0.02 for SYN3527 vs Saline Control on Day 9). Tumor weight shown in FIG. 7D at day 9 following administration of STING agonist producing bacteria shows significant tumor regression compared to saline control treatments (p<0.02). Flow cytometric analysis of lymphocytes from the tumor draining lymph node were performed by placing cells into single cell suspension and straining with the following antibodies: Anti-CD4-APC, TCR-beta-PECy7, CD8-alpha-BV785, CD25-BV650, and FoxP3-PE (all from Biolegend). Tumor regression correlates with an increase in total T cell numbers in the tumor draining lymph node (TDLN), as measured by flow cytometric analysis (p<0.03 for SYN3527 vs Saline Control), which may indicate activation and expansion of tumor specific T cells (FIG. 7E).

To evaluate of tumor colonization and bacterial growth following SYN-STING treatment of B16F10 tumors, B16F10 tumors were essentially treated as described above. On day 9 post initiation of treatment tumors were homogenized. Homogenates were serially diluted and plated on LB agar plates to calculate the number of viable bacteria (or colony forming units) per gram tissue within tumors. SYN-STING samples were plated on agar plates containing the appropriate antibiotic to ensure bacteria did not lose expression of STING circuit. Results are shown in FIG. 7H.

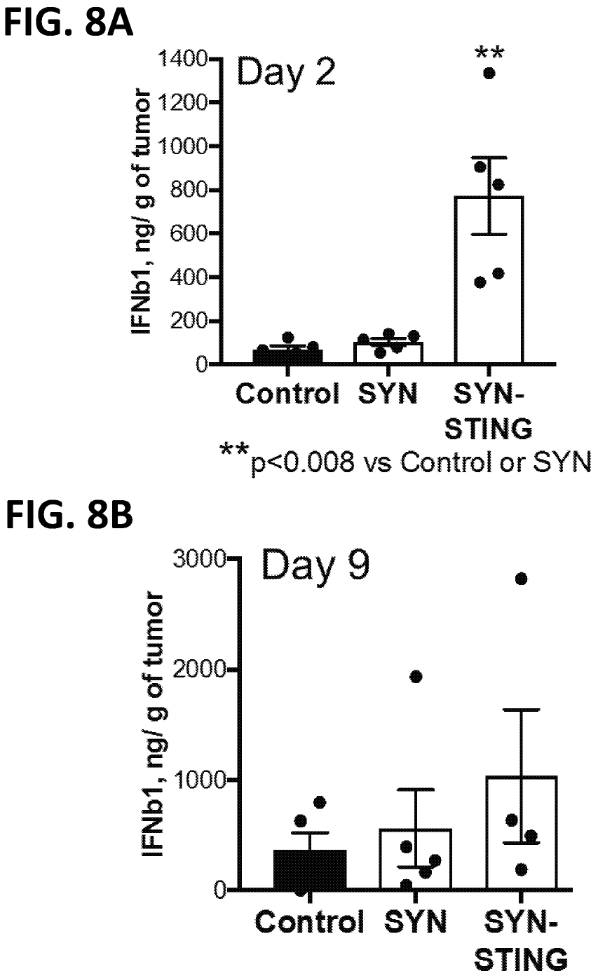

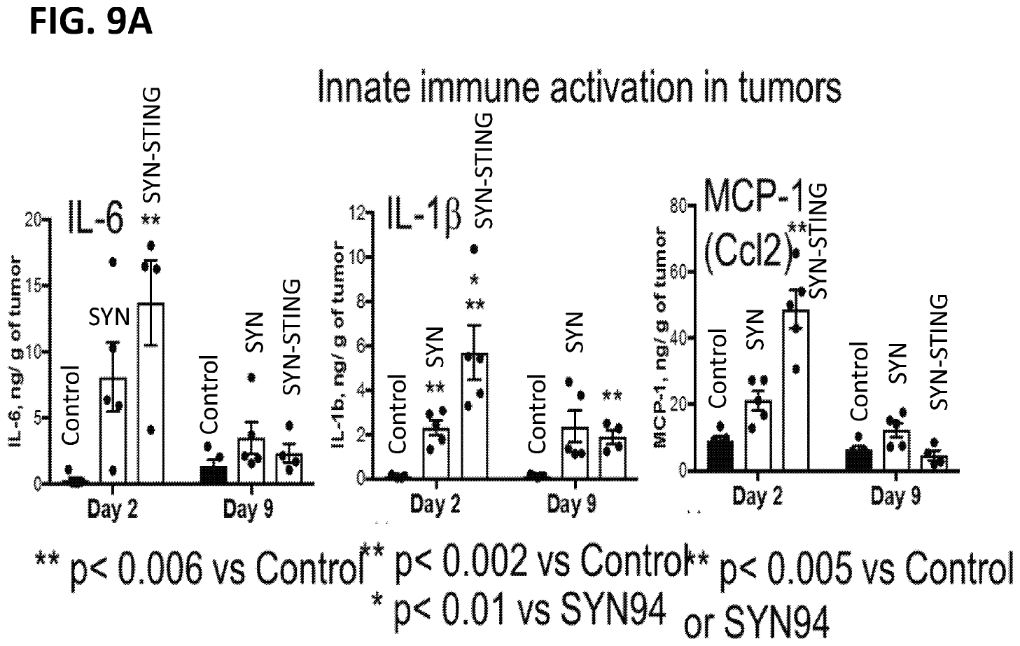

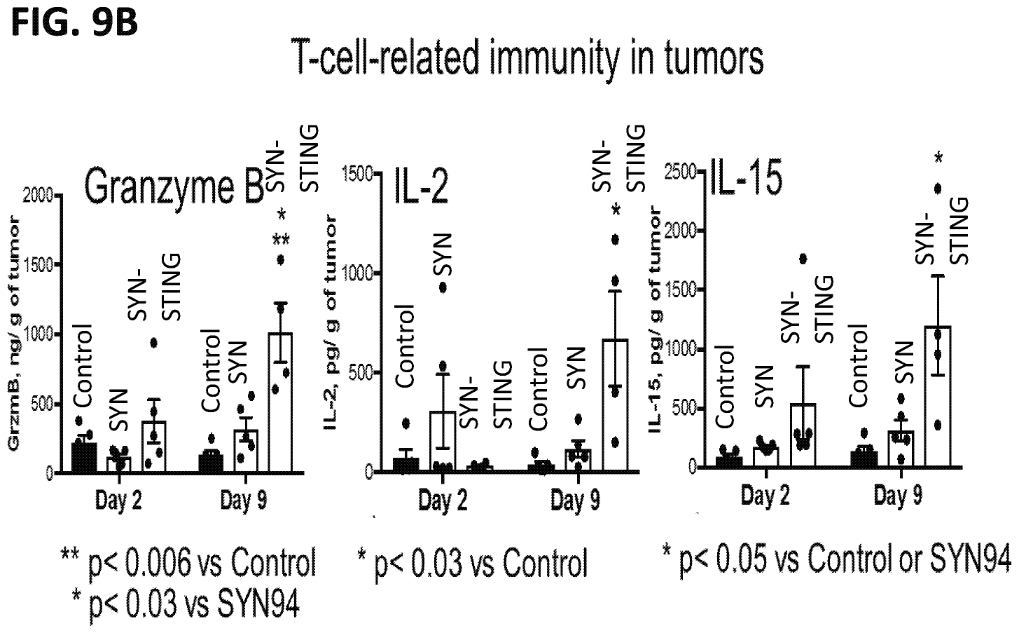

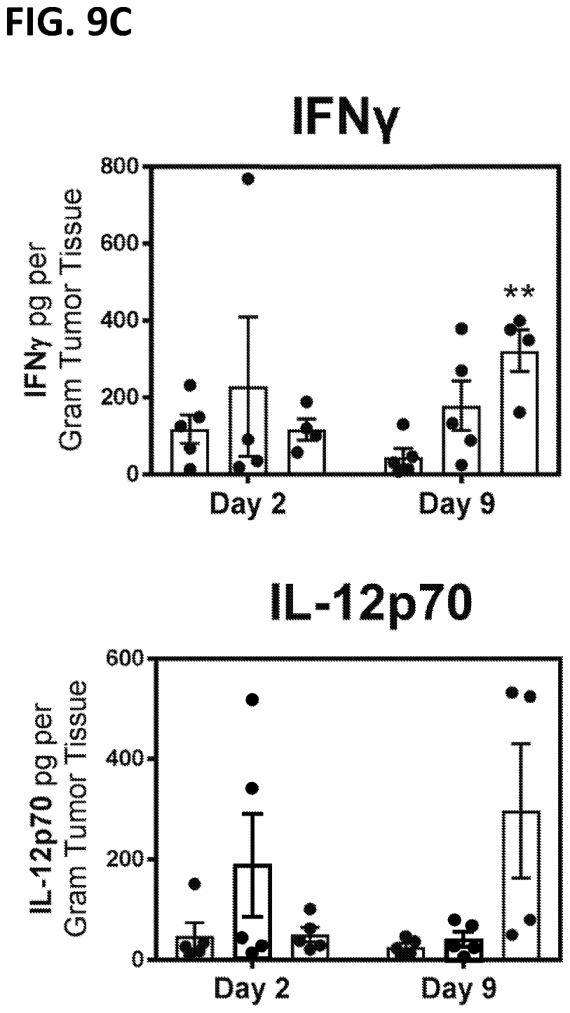

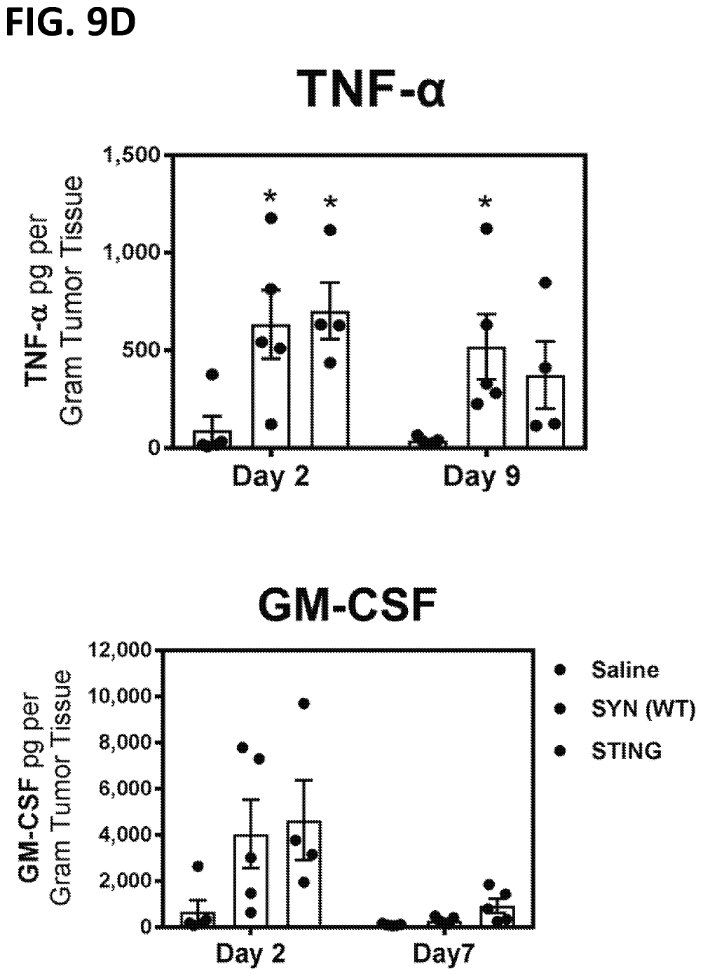

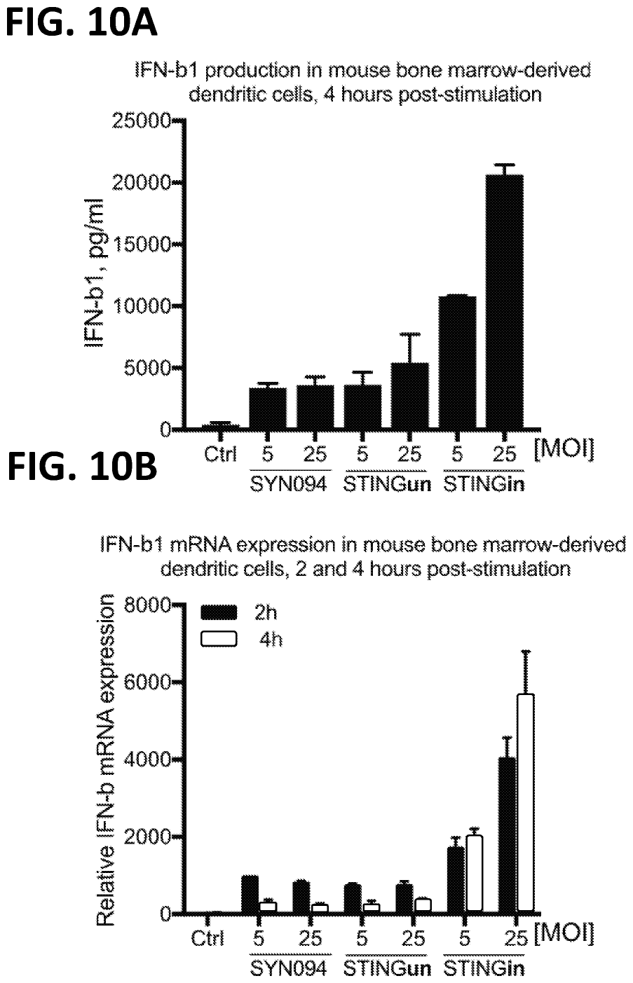

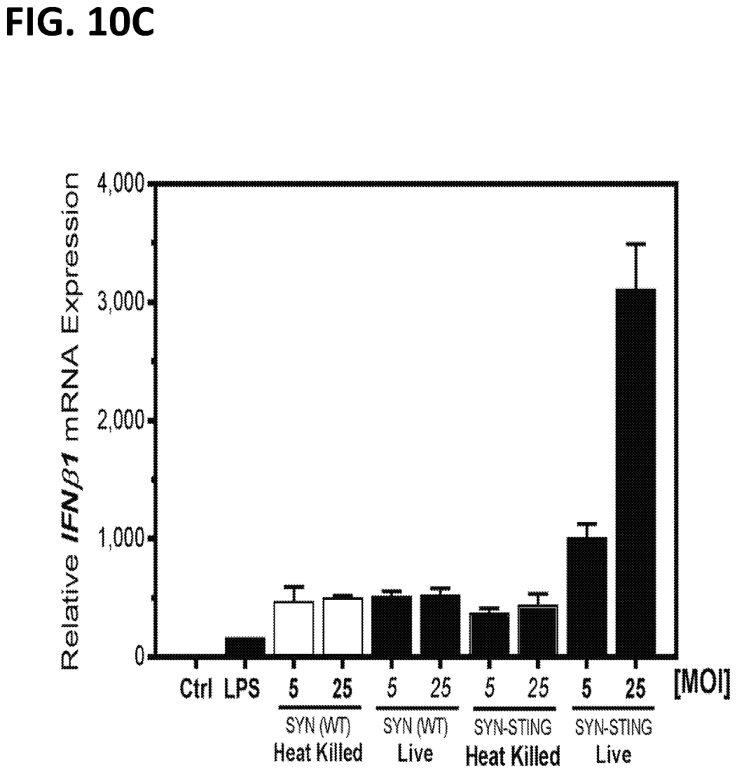

For cytokine analysis, a Luminex assay was performed according to manufacturer's instructions. To determine the activation of the innate immune system by the bacterially produced STING agonist, levels of INF-beta1, IL-6, IL-1beta, and MCP-1 (Cc12)) were assessed and results for day 2 and day 9 are shown in FIG. 8A and FIG. 8B. Results indicate that STING agonists produced by SYN3527 are able to increase significantly the IFN-beta production at day 2 in the tumor (P<0.008 vs control or SYN94), as well as induce the general innate immune response over saline and Nissle alone at day 2, but not day 9, as shown by IL-6 (p<0.006 vs Control), TNFα (** p<0.002 vs Control, * p<0.01 vs SYN94) and MCP-1 induction. The innate response within the tumor at day 2 was switched to a T-cell related response at day 9, as shown by the production of T-cell related cytokines granzyme B (p<0.006 vs Control; <0.03 vs SYN94, IL-2 (p<0.03 vs Control) and IL-15 (p<0.05 vs Control or SYN94) (FIG. 8B). FIG. 9 shows cytokines upregulated by bacterial injection (i.e., both WT and SYN-STING).

### Example 38. Activity of Adenosine Consuming Strain in Combination with Systemic Anti-PD-1 and Anti-CTLA-4 in MC38 Tumor Model

The ability of the adenosine consuming strain SYN1656 to augment the anti-tumor response of combined anti-CTLA4 and anti-PD-1 was assessed in the C57BL/6-MC38 syngeneic tumor model.

To produce cells used in the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with MC38 tumors, and mice injected intratumorally with the adenosine consuming bacteria and intraperitoneally with anti-CTLA-4 and anti-PD-1 antibodies according to the study design in Table 54. MC38 cells were implanted (1×105/mouse/100 μL) SC into the right flank of each animal on day −9. Tumor growth was monitored; when the tumors reached ˜50-80 mm\hat{ }3 on day 1, mice were randomized into treatment groups as shown in Table 50.

Tumor volumes and body weights were recorded three times in a week with a gap of 1-2 days in between two measurements.

TABLE 50
Study design

| | | Treatment 1 | | | | Treatment 2 | | | | |
|---|---|---|---|---|---|---|---|---|---|---|
| | | Test | | | | Test | | | | Treatment 3 |
| Group | N | Article | Route | Dose | Schedule | Article | Route | Dose | Schedule | Compound |
| 1 | 12 | Anti- | i.p. | 200 ug | Day 2, 5, | Anti- | i.p. | 100 ug | Day 2, 5, | NA |
| | | PD-1 | | | 8, 11, 14, | CTLA-4 | | | 8, 11, 14, | |
| | | Isotype | | | 17, 20 | Isotype | | | 17, 20 | |
| | | Control | | | | Control | | | | |
| 2 | 12 | Anti- | i.p. | 200 ug | Day 2, 5, | Anti- | i.p. | 100 ug | Day 2, 5, | NA |
| | | PD-1 | | | 8, 11, 14, | CTLA-4 | | | 8, 11, 14, | |
| | | | | | 17, 20 | | | | 17, 20 | |
| 3 | 12 | Anti- | i.p. | 200 ug | Day 2, 5, | Anti- | i.p. | 100 ug | Day 2, 5, | SYN094, 5e6 bacteria, |
| | | PD-1 | | | 8, 11, 14, | CTLA-4 | | | 8, 11, 14, | i.t., BIWx3 |
| | | | | | 17, 20 | | | | 17, 20 | Starting on Day 1 |
| 4 | 12 | Anti- | i.p. | 200 ug | Day 2, 5, | Anti- | i.p. | 100 ug | Day 2, 5, | SYN825, 5e6 bacteria, |
| | | PD-1 | | | 8, 11, 14, | CTLA-4 | | | 8, 11, 14, | i.t., BIWx3 |
| | | | | | 17, 20 | | | | 17, 20 | Starting on Day 1 |
| 5 | 12 | Anti- | i.p. | 200 ug | Day 2, 5, | Anti- | i.p. | 100 ug | Day 2, 5, | SYN1656, 5e6 bacteria, |
| | | PD-1 | | | 8, 11, 14, | CTLA-4 | | | 8, 11, 14, | i.t., BIWx3 |
| | | | | | 17, 20 | | | | 17, 20 | Starting on Day 1 |

Results show that the adenosine consuming strain has the ability to improve anti-CTLA-4/anti-PD-1 antibody-mediated anti-tumor activity in the MC38 model. Specifically, in the anti-PD-1/anti-CTLA-4 group, 5 out of 12 mice responded to the treatment, including 1 out of 12 with a complete response. In the anti-PD-1/anti-CTLA-4 plus SYN1656 group, the same number (5 out of 12) responded, but at least 4 out of 5 responders were complete responders.

### Example 39. Generation of a Strain for the Conversion of 5-FC to 5-FU

5-FU is a common chemotherapeutic limited by its systemic toxicity. However, 5-FC is much better tolerated. Using codA (cytosine deaminase) or a fusion of the codA (cytosine deaminase) and upp (uracil phosphoribosyltransferase), 5-FC can be converted into active drug form—5F-UMP—in the tumor, minimizing associated side effects.

To generate 5-FU producing strains, codA (cytosine deaminase) or a fusion of the codA (cytosine deaminase) and upp (uracil phosphoribosyltransferase) was cloned into a p15 vector under the control of the Ptet promoter; to generate the strain as described in Table 51.

TABLE 51

| Strain: | Genotype |
|---|---|
| SYN3529 | Nissle pUC-Kan-tet-CodA (cytosine deaminase) |
| SYN3620 | Nissle p15A Ptet-CodA::Upp fusion |

### Example 40. In Vitro Conversion of 5-FC to 5-FU

The ability of the newly generated strain to convert 5-FC to 5-FU was first assessed in vitro.

E. coli Nissle strains SYN3529, SYN3620 described above and SYN94 control (wild type Nissle with streptomycin resistance), were grown overnight in LB medium. Cultures were diluted 1:50 in M9 minimal media supplemented with 0.5% glucose (w/v) and grown shaking (350 rpm) at 37° C. for 2 hours, at which time anhydrous tetracycline (ATC) was added to cultures at a concentration of 100 ng/mL to induce expression of CodA or CodA-Upp fusion. After 2 hours of induction, cultures were spun down, media was aspirated and the cell pellets were resuspended into M9+0.5% glucose (w/v)+ATC (100 ng/mL)+10 mM 5-fluorocytosine (Sigma-Aldrich®). These cultures were then returned to the 37° C. incubator and allowed to incubate with shaking for additional 2 hours at which point samples were removed for LC/MS analysis of 5-FU production. Samples were centrifuged for 20 minutes at 5000 RPM to separate cellular and extracellular fractions. Cell pellets were then used to determine intracellular 5-FC and 5-FU and the media supernatants were used to determined extracellular accumulation of 5-FU or consumption of 5-FC.

Results are shown in FIG. 34A and FIG. 34B. and indicate that engineered strains were able to degrade 5-FC (FIG. 34A) and to produce 5-FU (FIG. 34B) at a rate higher than that of the wild type control strain. Since there was no available standard for 5-FUMP, we were unable to measure the accumulation of 5-FUMP from SYN3620.

### Example 41. In Vivo Activity of 5F-C to 5-FU Converter in the CT26 Tumor Model

To determine In Vivo activity and efficacy of 5-FC to 5-FU converting strains SYN3529 (comprising pUC-Kan-tet-CodA (cytosine deaminase)) and SYN3620 (comprising pUC-Kan-tet-CodA::Upp fusion), tumor volume was assessed and compared to PBS control in the CT26 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with CT26 tumors, injected intratumorally with bacteria producing enzymes capable of converting 5-FC into 5-FU or vehicle control, and dosed with 5-FC according to the time line described below and in FIG. 35A. Tumor volumes were measured at various time points, while tumors were weighed and processed at the conclusion of the experiment to evaluate relative consumption of 5-FC as a measure of the strains bioactivities.

Briefly, CT26 cells were implanted (1×10\hat{ }6 cells/mouse in PBS) SC into the right flank of each animal on day −15. Tumor growth was monitored until the tumors reached ˜150-450 mm\hat{ }3. On day 0, mice were randomized into groups (N=5 per group) for intratumor dosing as follows: PBS (group 1, vehicle control), SYN3529 (group 2, 1×10\hat{ }7 CFU), SYN3620 (group 3, 1×10\hat{ }7 CFU). Tumor sizes were measured and mice were injected I.T. with bacteria or PBS on day 0, 2, and 5, followed by ATC (lug I.P.) 4 hours later. Starting on day 3, 5-FC (500 mg/kg) was administered daily via IP injection. Mice were sacrificed on day 6 for final analysis.

Average tumor volumes at day 0, 2, and 5 are shown in FIG. 35B and for individual mice in FIG. 35C, and indicate that administration of either SYN3529 or SYN3560 alongside 5-FC results in a blunting of tumor growth over this time period compared to PBS control. Tumor weight shown in FIG. 35D at day 6 following administration of bacteria, day 3 post 5-FC dosing, shows reduction in tumor mass compared to PBS control treatments. Mass spectrometry analysis of tumor homogenates demonstrate a reduction in 5-FC levels for bacterially colonized tumors compared to PBS controls, suggesting in situ conversion of 5-FC to its active form 5-FU (FIG. 35E).

### Example 42. Generation of a Combined Kynurenine Consumer and a STING Agonist Producer and In Vitro Measurement of Strain Activity

To generate a strain that consumes kynurenine and produces a STING agonist, SYN2028 (comprising Nissle HA3/4::PSynJ23119-pKynase TrpE::CmR) was transformed with the construct previously used in SYN3527 (DacA cloned into a p15 vector under the control of the Ptet promoter); to generate the strain as described in Table 52.

TABLE 52

| Strain: | Genotype |
|---|---|
| SYN2028 | Nissle HA3/4::PSynJ23119-pKynase TrpE::CmR |
| SYN3527 | Nissle p15A Ptet-DacA (listeria monocytogenes cyclic di |
| | AMP synthase) |
| SYN3831 | Nissle HA3/4::PSynJ23119-pKynase TrpE::CmR; p15a |
| | Ptet - DacA |

Next, the ability of the newly generated strain to consume kynurenine and to produce a STING agonist was assessed in vitro. E. coli Nissle strains SYN094 (wild-type control), SYN2028 (KYN), SYN3527 (STING) and SYN3831 (KYN+STING) were grown overnight in LB medium containing appropriate antibiotics. Cultures were diluted 1:25 in M9 minimal media supplemented with 0.5% glucose (w/v) and appropriate antibiotics or LB and antibiotics and grown shaking (350 rpm) at 37° C. for 2 hours.

For measurement of cyclic-di-AMP production, cultures in the M9 media were diluted with the same M9 supplemented media to an optical density (600 nm) of 0.5 at which time anhydrous tetracycline (ATC) was added to cultures at a concentration of 100 ng/mL to induce expression of DacA. Cells were induced for a further 4 hours to allow accumulation of cyclic-di-AMP.

Samples were removed for LC/MS analysis of cyclic dinucleotide production. Samples were centrifuged for 5 minutes at 10000×g to separate cellular and extracellular fractions. Cell pellets were then used to determine intracellular cyclic-di-AMP. Concentrations were determined via LC/MS. For quantifying kynurenine consumption, the LB cultures were spun down and resuspended in LB with 100 μM L-kynurenine (Sigma-Aldrich®) to an optical density (600 nm) of 1.0. These cultures were then placed at 37° C. with shaking for 4 hours to allow consumption of kynurenine. For kynurenine consumption measurements, samples were removed from each culture and spun for 5 minutes at 10000×g to separate the cellular and extracellular fractions. Media supernatant was removed and used to determine consumption of kynurenine via LC/MS analysis. Results shown in FIG. 17A indicate that the engineered strain SYN3831 was able to produce cyclic-di-AMP similar to SYN3527, where it's kynurenine-consuming parent, SYN2028, was not able to produce cyclic-di-AMP. Results of FIG. 17B indicate that the new combo strain SYN3831 also retained the ability to consume kynurenine similarly to it's parent

SYN2028. Taken together these results support that in vitro the combo strain SYN3831 possesses the ability to both produce the STING agonist cyclic-di-AMP, as well as consume the AHR agonist, kynurenine.

### Example 43. Functional Assay for Secreted IFN-Gamma

Next, studies were conducted to demonstrate that IFN-gamma secreted from genetically engineered bacteria is functional. A cell-based assay was employ based on increased phosphorylation of STAT1 upon binding of IFNgamma to its receptor. Bioactivity of IFN-gamma can be determined by quantification of STAT1 phosphorylation via flow cytometry.

Next, studies were conducted to demonstrate that IFN-gamma secreted from genetically engineered bacteria is functional. A cell-based assay was employ based on increased phosphorylation of STAT1 upon binding of IFNgamma to its receptor. Bioactivity of IFN-gamma can be determined by quantification of STAT1 phosphorylation via flow cytometry.

Briefly, mouse RAW264.7 cells were treated with supernatants from murine IFNg expressing bacteria (SYN3543 comprising PAL::Cm p15a Ptet-87K PhoA-mIFNg) for 15 min. Treated cells were fixed in paraformaldehyde based solution followed by harsh permeabilization in 90% methanol. Modulation of phospho-STAT1 was quantified by flow cytometry, and results are shown in FIGS. 39A and 39B.

FIG. 39A and FIG. 39B, shows bioactivity of SYN3543 in two independent assays.

### Example 44. Activity of CD40L Secreting Strain SYN3367 In Vivo

To determine the in vivo activity of the CD40L secreting strain SYN3367 (comprising PAL::Cm pUC-tet-PhoA-mCD40L 112-260; referred to in this Example and FIG. 36 as SYN-CD40L), intratumoral antigen presenting cell (APC) activation was assessed by flow cytometry and compared to treatment with either SYN1557 (DOM mutant; referred to in this Example and FIG. 36 as SYN) or recombinant mouse CD40L (R&D Systems) in the CT26 tumor model.

To produce bacterial cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration-tested by serial plating.

Briefly, CT26 cells were implanted (1×10\hat{ }6 cells/mouse in PBS) SC into the right flank of each animal. Tumor growth was monitored; when the tumors reached ˜80-100 mm\hat{ }3, mice were randomized into groups (N=6 per group) for intratumor dosing as follows: SYN1557 (leaky phenotype DOM mutant, group 1, 1×10\hat{ }7), SYN3367 (mCD40L secreter, group 2, 1×10\hat{ }7), and recombinant mCD40L (1 ug; Group 3). On day 0, mice were injected I.T. with bacteria. On day 1, 4, and 7, all groups were injected with 1 ug of ATC via IP injection (in three pulses). On day 1, 4, and 7, Group 3 was treated with 1 ug recombinant CD40L via I.T. injection. On day 8, all mice were sacrificed with 3 mice utilized to analyze activation of intratumoral APCs via flow cytometry and 3 mice utilized to measure bacterial colonization via CFU plating.

Three representative tumors were homogenized, tumor homogenates were serially diluted and plated on LB Agar plates containing the appropriate antibiotics in order to measure the colony forming unit concentration. As depicted in FIG. 36B, SYN-CD40L colonized tumors to a similar extent as SYN reaching up to 1×10⁸ CFUs/gram tumor. Flow cytometric analysis of intratumoral APCs from three representative tumors were performed by digesting tumors in a mixture of DNAse and Liberase TL (Sigma) for 30 minutes at 37° C., placing cells into single cell suspension and straining with the following antibodies: Anti-MHCII(IA/IE), CD45.2, CD40, Gr1, CD197(CCR7), CD11b, CD11c (all from Biolegend). Treatment of CT26 tumors with SYN-CD40L resulted in higher levels of CCR7 expression on intratumoral dendritic cells (p<0.04 for SYN-CD40L vs. SYN control) with a trend towards higher expression on macrophages as well (FIG. 36C). Additionally, a trend towards higher expression for CD40 was observed on both dendritic cells and macrophages in the tumor. These results suggest that treatment with SYN-CD40L results an increased activation of critical APC subsets within the tumor.

### Example 45. Activity of hTNFa in CT26 Tumors

To determine In Vivo activity of hTNFa expressing strain SYN2304 (comprising PAL::CM p15a TetR Ptet-PhoA-TNFα; referred to in this Example and FIG. 38 as SYN-TNFα) tumor volume was assessed and compared to SYN1557 (DOM mutant; referred to in this Example and FIG. 38 as SYN) in CT26 tumors.

To produce bacterial cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Briefly, CT26 cells were implanted (1×10\hat{ }6 cells/mouse in PBS) SC into the right flank of each animal. Tumor growth was monitored; when the tumors reached ˜50-80 mm\hat{ }3, mice were randomized into groups (N=5 per group) for intratumor dosing as follows: SYN (leaky phenotype DOM mutant, group 1, 1×10\hat{ }7), and SYN-TNFα (TNFα secreter, group 2, 1×10\hat{ }7). On days 0 and 4, mice were injected I.T. with bacteria. On days 1 and 4, both groups were given 1 ug of ATC via IP injection (in two pulses). Tumor size was measured 2× times a week. Tumors were homogenized, tumor homogenates were serially diluted and plated on LB Agar plates containing the appropriate antibiotics in order to measure the colony forming unit concentration. As depicted in FIG. 38B, SYN-TNFα colonized tumors to a similar extent as SYN, reaching up to 1×10⁸ CFUs/gram tumor. Intrumoral expression of hTNFa was only detected in SYN-TNFα treated CT26 tumors, as measured by hTNFa ELISA (R&D Systems) (FIG. 38C). Results in FIG. 38D show significantly reduced tumor growth for CT26 tumors treated with SYN-TNFα at day 7 post first bacterial dose compared to treatment with SYN (P<0.02). Taken together these results demonstrate the SYN-TNFα is capable of colonizing and persisting in CT26 tumors, where they express detectable levels of hTNFa and result in a significant reduction in tumor growth.

### Example 46. Activity of mIFNgamma Secreting Strain SYN3367 In Vivo

To determine the in vivo dynamics of the IFNgamma expressing strain SYN3367 (comprising PAL::Cm p15a Ptet-87K PhoA-mIFNg; referred herein as SYN-IFNγ) CT26 tumors were treated and compared to SYN1557 (DOM mutant; referred herein as SYN) treated tumors.

To produce bacterial cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Briefly, CT26 cells were implanted (1×10\hat{ }6 cells/mouse in PBS) SC into the right flank of each animal. Tumor growth was monitored; until tumors reached ˜50-80 mm\hat{ }3 when mice were randomized into groups (N=6 per group) for intratumor dosing as follows: SYN (leaky phenotype DOM mutant, group 1, 1×10\hat{ }7) and SYN-IFNγ (IFNgamma secreter, group 2, 1×10\hat{ }7) On day 0 and day 3 mice were dosed with the appropriate bacterial strain. On days 1, 4 and 7 4, all mice were injected I.P. with 1 ug ATC (in three pulses). On day 8, all mice were sacrificed with 3 mice utilized to measure intratumoral bacterial colonization via CFU plating and 3 mice utilized to analyze intratumoral IFNγ production by ELSIA. Tumors were homogenized, tumor homogenates were serially diluted and plated on LB Agar plates containing the appropriate antibiotics in order to measure the colony forming unit concentration. As depicted in Figure XB, SYN-IFNγ colonized tumors to a similar extent as SYN, reaching up to 1×10⁸ CFUs/gram tumor. Intrumoral expression of IFNγ was only detected in SYN-IFNγ treated CT26 tumors, as measured by murine IFNγ ELISA (R&D Systems) (FIG. 40C). Taken together these results demonstrate the SYN-IFNγ is capable of colonizing and persisting in CT26 tumors, where they express detectable levels of IFNγ following induction with ATC.

### Example 47. Functional Assay for Secreted CXCL10

To determine whether human CXCL10 secreted from any of the strains described above is functional, a Chemotaxis Assay was performed, essentially as described in Mikucki et al. (Mikucki, et al., Non-redundant Requirement for CXCR3 Signaling during Tumoricidal T Cell Trafficking across Tumor Vascular Checkpoints; Nat Commun. 2015; 6: 7458, the contents of which is herein incorporated by reference in its entirety). In this assay, human CD8+ T cells (8 days post anti-CD3/CD28 expansion) were cell trace violet labeled and transferred into a 24-well transwell plate (5 uM pore) with T cells on top and bacterial supernatants on bottom, in the presence or absence of anti-hCXCR3. Cells were incubated for 3 hrs, and migrated cells recovered from the bottom of wells were counted by flow cytometry.

To prepare the bacterial supernatants, strains (SYN1557 control strain and SYN2942 (comprising PAL::Cm Ptet-87K PhoA (ECOLIN_02255))) were thawed and grown over night, then induced to express hCXCL10 by addition of tetracycline for 3 hours. Supernatants were sterifiltered and then used as noted below. To prepare the T cells, pre-isolated primary human CD8 T cells (AllCells) were counted, harvested and resuspended in T cell media, approximately 8 days post stimulation with anti-CD3/CD28beads. 24-well trans well plates with space 0.5 ml on bottom and 0.1 nil on top of the wells and 5 uM pore size were used for the assay. To prepare bacterial supernatants for the bottom of the well, supernatants from SYN2942 were diluted in supernatants taken from SYN1557 control bacteria to generate mixtures containing 100%, 33%, 11%, 3.7%, and 0% SYN2942. To prepare the top of the well, 100 uL of T cells were added to the top well (containing approximately 2.5×10 cells). Anti-CXCR3(mouse IgG1; clone G025H7) was added to the control wells at a final concentration of 1 ug/ml). Plates were placed in 37 C incubator for 3 hours before analysis. For analysis, the contents of each bottom well were transferred to two wells of a 96-well plate. Plates were spun down, two wells were combined, resuspended in PBS, and analyzed on a MACSQuant flow cytometer to quantify the number of migrated cells. Primary human T cells trafficked towards SYN2942 supernatants in a dose-dependent manner which was abrogated by blockade of CXCL10's receptors CXCR3. This data (not shown) would suggest that the hCXCL10 produced by SYN2942 is biologically active and abundant in sufficient concentrations to trigger primary T cell trafficking in vitro.

### Example 48. Evaluation of Efficacy of SYN3527 Treatment in a Balb/c-A20 Tumor Model

To determine In Vivo activity and efficacy of SYN3527 (comprising plasmid-based tet-inducible dacA from Listeria monocytogenes), over time at three different doses and compared to PBS control in the c-A20 tumor model (A20 B-cell lymphoma).

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Female Balb/c mice 6 weeks of age were implanted with A20 tumors, injected intratumorally with three different doses of bacteria producing enzymes capable of producing c-diAMP. Tumor volumes were measured at various time points, while tumors were weighed and processed at the conclusion of the experiment.

Briefly, A-20 cells were implanted (2×10 5/mouse/100 μL in PBS) SC into the right flank of each animal on day −15. Tumor growth was monitored until the tumors reached ˜100 mm\hat{ }3. On day 0, mice were randomized into groups (N=8 per group) for intratumor dosing as follows: PBS (group 1, vehicle control), SYN3527 (group 2, 1×10\hat{ }7 CFU), SYN3527 (group 3, 5×10\hat{ }7 CFU), and SYN3527 (group 4, 5×10\hat{ }8 CFU). Tumor sizes were measured and mice were injected I.T. with bacteria or PBS on day 0, 2, and 5, followed by ATC (lug I.P.) 4 hours later.

Animals were dosed with appropriate bacteria based on group or saline (to control for injection) on days 0, 3, and 7. Four hours after dosing with the bacteria, mice were treated with 10 ug ATC (anhydrotetracycline) via intraperitoneal injection. Tumor volumes and body weights were recorded three times in a week with a gap of 1-2 days in between two measurements.

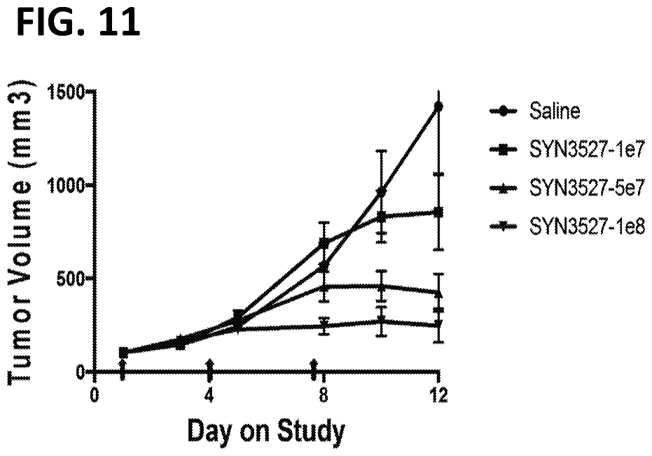

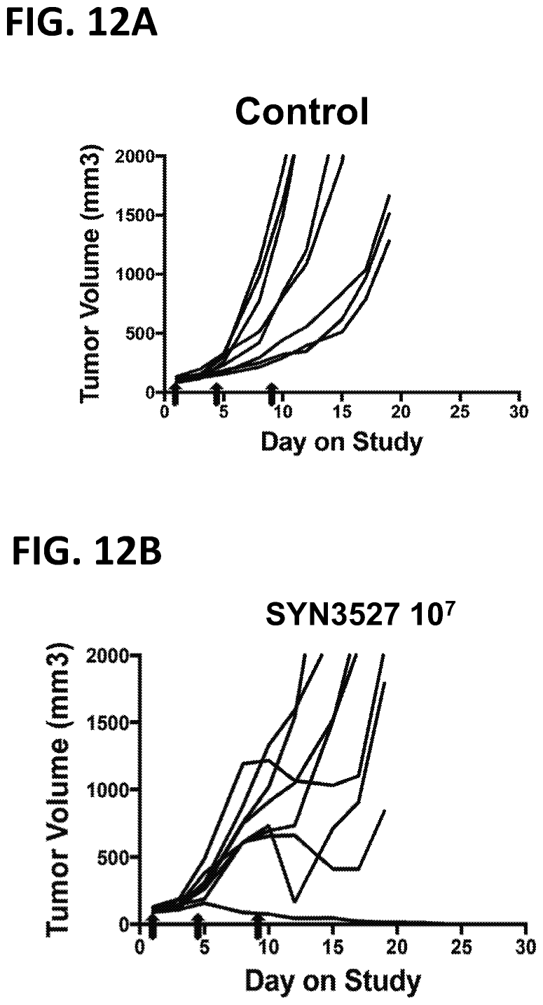

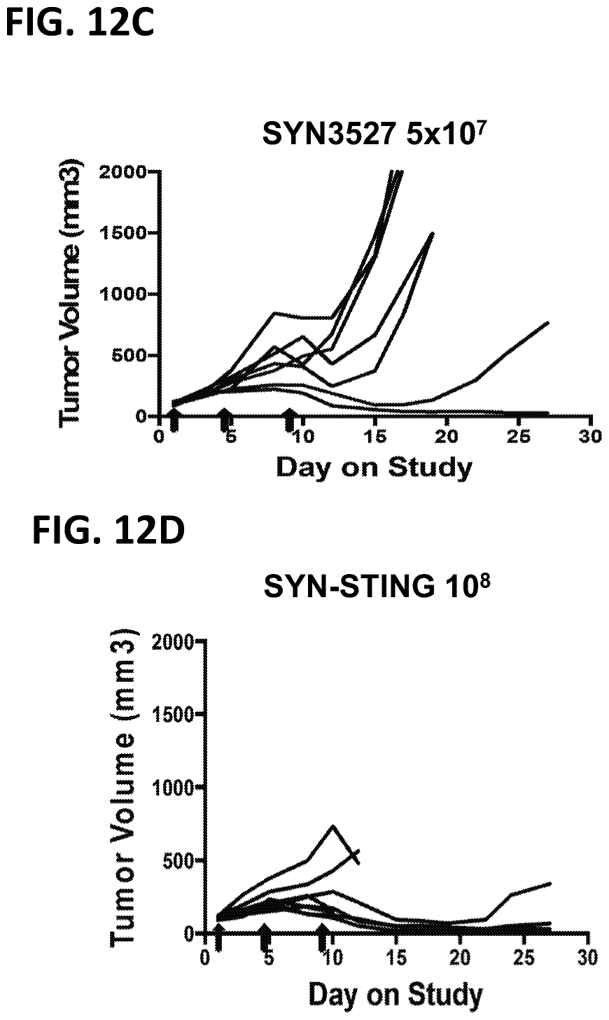

Tumor volume at day 1, 4, and 12 are shown in FIG. 11 and for up to 27 days in individual mice in FIG. 12A (saline control), FIG. 12B (1×10\hat{ }7 CFU), FIG. 12C (5×10\hat{ }7 CFU), and FIG. 12D (5×10\hat{ }8 CFU). Results indicate that administration of SYN3527 drives dose-dependent tumor control in A20 lymphoma model.

### Example 49. Combining STING Initiator and Kyn Sustainer: Improved Efficacy with aPD1 Checkpoint Inhibition

Next combination of an immune initiator/sustainer pair was tested, alone or in combination with checkpoint inhibitor therapy. To determine the efficacy of the initiator STING producing strain SYN3527 (E. coli Nissle comprising plasmid-based, tetracycline-inducible p15a Ptet-DacA from Listeria monocytogenes) in combination with the sustainer Kynurenine consuming strain SYN2028 (comprising integrated kynureninase under constitutive promoter and ΔTrpE), tumor volume, survival and body weight was monitored in a B16F10 tumor model, which is considered an immunologically “cold” tumor model. To assess whether checkpoint inhibition would further improve efficacy of the initiator/sustainer pair, the effect of the addition of an anti-PD1 antibody administered systemically was also assessed. Mice were treated with 2 doses of the STING agonist producing strain (initiator) followed by either anti-PD1, Kynurenine-consuming strain or the combination (sustainer).

To produce cells for the study (SYN3527 and SYN2028), overnight cultures were used to inoculate 500 mL LB medium with antibiotic. The strains were incubated with shaking at 37 C until the cultures reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with B16F10 tumors, and mice injected intratumorally with bacteria producing STING agonists and subsequently with bacteria that consume kynurenine, according to the time line described below. Briefly, B16F10 cells were implanted (2e5 cells/mouse in PBS) SC into the right flank of each animal on day −9. On day day −3 tumor growth was monitored; when the tumors reached ˜50-80 mm³ on day 0, mice were randomized into experimental groups (N=10 per group) for intratumor dosing as follows: Group 1: Saline+Isotype control; Group 2: Saline+anti-PD1 antibody; Group 3: SYN3527 [1e7]+Isotype control; Group 4: SYN3527 [1e7]+anti-PD1 antibody; Group 5: SYN3527 [1e7]->SYN3527:SYN2028 [1e7]->SYN2028 [1e7]+Isotype control; Group 6: SYN3527 [1e7]->SYN3527:SYN2028 [1e7]->SYN2028 [1e7]+anti-PD1 antibody; Group 7: SYN3527 [1e6]->SYN3527 [1e7]+Isotype control; Group 8: SYN3527 [1e6]->SYN3527 [1e7]+anti-PD1 antibody. Isotype control and anti-PD1 antibody were dosed at 200 ug per injection Animals were dosed with appropriate bacteria based on group on day 1, day 5, and day 8. Four hours after each bacterial dose, mice were treated with 10 ug ATC (anhydrotetracycline) via intraperitoneal injection. Bacterial dosing was stopped for complete responders. Table 53 provides a summary of the treatment timeline. Results are shown in Table 54.

TABLE 53
Summary of Treatment Timeline

| | | | | 2X weekly, |
|---|---|---|---|---|
| | | | | starting at Day |
| | | | | 8 until end of |
| Group | Day 1 | Day 5 | Day 5 | study (Day 21) |
| 1 | Saline | Randomize prior | Saline + Iso | Saline + Iso |
| 2 | | to day 5 | Saline + PD1 | Saline + PD1 |
| | | treatment | | |
| 3 | SYN3527 | Randomize prior | SYN3527 | Saline + Iso |
| | [1e7] | to day 5 | [1e7] + Iso | |
| 4 | | treatment | SYN3527 | Saline + PD1 |
| | | | [1e7] + PD1 | |
| 5 | | | SYN3527 | SYN2028 |
| | | | [5e6] + | [1e7] + Iso |
| | | | SYN2028 | |
| | | | [5e6] + Iso | |
| 6 | | | SYN3527 | SYN2028 |
| | | | [5e6] + | [1e7] + PD1 |
| | | | SYN2028 | |
| | | | [5e6] + PD1 | |
| 7 | SYN3527 | Randomize prior | SYN3527 | SYN3527 |
| | [1e6] | to day 5 | [1e7] + Iso | [1e7] + Iso |
| 8 | | treatment | SYN3527 | SYN3527 |
| | | | [1e7] + PD1 | [1e7] + PD1 |

TABLE 54
Study Results

| | Complete Response | Partial Response | |
|---|---|---|---|
| | (CR) (Tumor | (PR) (<50 mm³ | ORR |
| Group | Regression) | tumors) | (CR + PR) |
| Saline | 0 out of 5 | 0 out of 5 | 0 out of 5 |
| | (0%) | (0%) | (0%) |
| aPD-1 | 0 out of 5 | 0 out of 5 | 0 out of 5 |
| | (0%) | (0%) | (0%) |
| STING | 3 out of 16 | 0 out of 16 | 3 out of 16 |
| | (18.75%) | (0%) | (18.75%) |
| STING + aPD-1 | 2 out of 16 | 1 out of 16 | 3 out of 16 |
| | (12.5%) | (6.25%) | (18.75%) |
| STING → Kyn | 2 out of 15 | 0 out of 15 | 2 out of 15 |
| | (13.3%) | (0%) | (13.3%) |
| STING → Kyn + | 4 out of 16 | 2 out of 16 | 6 out of 16 |
| aPD-1 | (25%) | (12.5%) | (37.5%) |

Dosing was discontinued for mice with tumors that demonstrated a complete or partial response to treatment and mice were monitored for durable response for 50 days. No tumors relapsed during this time period.

Next, animals showing CR were re-challenged by tumor re-implantation on the contralateral flank from the first tumor implant to assess whether the treatment with SYN3527 could confer immunological memory. Briefly, B16F10 cells were re-implanted on day 71 into the 11 complete responders from Groups 3-8 and compared to naïve animals (n=6). Results are shown in FIG. 12 and demonstrate a significant delay in tumor growth for mice which previously underwent a CR to the initial SYN3527 treatment, compared to naïve controls, implying the formation of immunological memory.

### Example 50. Assessment of Safety and Biocontainment Strategies: Proliferation of Auxotrophic Mutants in Tumors

The ability of auxotrophic mutant strains to proliferate in tumors was assessed. Three auxotrophic strains were generated: SYN1193 (ΔUraA::CM), SYN1534 or SYN1605 (ΔThyA::CM), SYN766 (ΔDapA::CM).

Proliferation of the auxotrophic strains were first assessed in the CT26 model. CT26 cells (˜1e6 cells/mouse in PBS) were implanted subcutaneously into the right flank of each animal (BalbC/J (female, 8 weeks-of-age)), and tumor growth was monitored for approximately 10 days. When the tumors reached ˜100-150 mm³, animals were randomized into groups for dosing.

For intratumoral injection, bacteria were grown in LB broth containing thymidine (3 mM), diaminopimelic (100 ug/mL DAP) acid or uracil as needed until reaching an absorbance at 600 nm (A600 nm) of 0.4 (corresponding to 2e8 colony-forming units (CFU)/mL) and washed twice in PBS. The suspension was diluted in PBS or saline so that 40 uL can be injected at the appropriate doses intratumorally into tumor-bearing mice.

Bacteria were suspended in PBS and mice were injected (1e6 cells/mouse) with 40 uL intratumorally as follows: Group 1-SYN94 (n=6), Group 2—SYN1193 (n=6), Group 3—SYN1534 (n=6), and Group 2—SYN766 (n=6). Tumor tissue was harvested at 24 and 72 hours. Tumor tissue homogenates were serially diluted and plated on LB agar plates (containing antibiotic and thymidine, diaminopimelic acid or uracil) to calculate the number of viable bacteria (or colony forming units) per gram tissue within tumors. Results in FIG. 18A show that strains comprising the ΔThyA and ΔUraA mutations are able to colonize and proliferate in the tumor, similar to the unmodified E. coli Nissle strain. Mutations in the pyrimidine pathways prevent bacteria from growing in the absence of a pyrimidine source, however, results indicate that sufficient levels of these nutrients are present in the tumor to allow colonization and growth. As such, these auxotrophic mutations are useful for biocontainment strategies by preventing growth in the absence of pyrimidines, while not negatively affecting growth and colonization properties and, by extension, effector production or efficacy within the tumor.

In contrast, the strain containing the ΔDapA mutation does not proliferate and lower numbers of bacteria are detected after 24 hours and 72 hours than were originally injected. Diaminopimelic acid (Dap) is an amino acid that is made by bacteria but not the mammalian host. It is a characteristic component of certain bacterial cell walls, e.g., of gram negative bacteria. Without diaminopimelic acid, bacteria are unable to form proteoglycan, and as such are unable to grow. As such, a DapA auxotrophy may affect bacterial growth and colonization of tumor and may present a particularly useful strategy to temporally modulate, fine tune timing, the extent of bacterial presence in the tumor and/or effector expression and production levels over time

Follow-on studies were conducted with ThyA auxotrophic strain in B16F10 and EL4 cells, shown in FIG. 18B and FIG. 18C. The ThyA auxotrophic strain was able to proliferate at a similar rate as the WT strain over the 72-hour time frame, recapitulating the growth patterns observed relative to the unmodified strain in the CT26 model.

### Example 51. Modulating Temporal Dose Through Chassis Modifications: Potential to Improve Therapeutic Window with DAP-STING Strain

Various methods to control levels and timing of effector production in vivo were assessed, each of which could be used individually or in combination (to allow for tighter control). Studies to assess the ability to control or fine-tune levels and timing of effector molecule production using inducible promoters (tetracycline, cumate, salicylate or hypoxia/anaerobiosis inducible) were conducted and are described below.

Additionally, the ability to regulate levels and timing of effector molecule production through controlling abundance of the genetically engineered bacteria was assessed. As described in the previous examples, it was advantageously found that DapA auxotrophic strains are unable to grow and colonize the tumor. Therefore, the DapA auxotrophy might present a means of gaining temporal control over bacteria load and dose and by extension effector payload delivery and abundance.

To test the effect of DapA on STING production, STING producer SYN4023 (E. coli Nissle comprising plasmid-based, tetracycline-inducible p15a Ptet-DacA from Listeria monocytogenes and ΔDapA) was generated. Next, the ability to modulate bacterial growth or colonization of the tumor and effector production was tested.

To determine the efficacy of treatments comprising the DAP-STING strain SYN4023 (E. coli Nissle comprising p15a Ptet-DacA from Listeria monocytogenes and ΔDapA), tumor volume and body weight was monitored in a B16F10 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with B16F10 tumors, and mice injected intratumorally with bacteria producing STING agonists, according to the time line described below. Briefly, B16F10 cells were implanted (2e5 cells/mouse in PBS) SC into the right flank of each animal on day −9. On day Day −3 tumor growth was monitored; when the tumors reached ˜50-80 mm³ on day 0, mice were randomized into groups (N=10 per group) for intratumor dosing as follows: saline (control, group 1), SYN4023 (DAP-STING) group 2, 1e7 CFU) and SYN4023 (DAP-STING) group 3, 1e8 CFU) Animals were dosed with appropriate bacteria based on group or saline (to control for injection) on days 1, 5 and 8. Four hours before the bacterial dose, mice were treated with 10 ug ATC (anhydrotetracycline) via intraperitoneal injection. Bacterial dosing was stopped for complete responders.

Tumor volumes at day 5, 8, and 12 are shown in FIG. 19A. Tumor volumes for individual mice in are shown in FIG. 19B, FIG. 19C and FIG. 19D. Results indicate that administration of SYN4023 at a dose of 1e8 results in rejection or control of tumor growth over this time period.

Additionally, these results indicate that limitation of bacterial growth through introduction of an DapA auxotrophy does not reduce efficacy of the strain.

### Example 52. Exclusion of Cytokine Release Syndrome

Cytokine release syndrome (CRS) is a form of systemic inflammatory response syndrome that arises as a complication of some diseases or infections and is also an adverse effect of some monoclonal antibody drugs, as well as adoptive T-cell therapies, such as CAR-T therapy. Cytokine release syndrome is caused by a large, rapid release of cytokines into the blood from immune cells during the course of an immunotherapy Similarly, systemic bacterial infection can lead to a rapid release of cytokines in a process known as sepsis. To ensure that intratumoral bacterial treatment would not lead to elevated levels of cytokines related to CRS or sepsis in the blood of mice, levels of cytokines in blood were measured upon treatment of tumors with the STING agonist producing bacteria and corresponding unmodified controls.

B16F10 tumors were implanted and monitored as described elsewhere herein, and mice were randomized and injected I.T. at time 0 according to groups as follows: saline, SYN3527 (STING, induced) at 1e7 CFU, SYN3527 (STING, uninduced) at 1e7 CFU, SYN4023 (STING DAP, induced) at 1e8 CFU or SYN94 (unmodified bacterium) at 1e7 CFU (n=12 per group). An LPS induced sepsis Control (500 ug in 100 uL PBS) was included in the study and administered intravenously. Four hours after each bacterial dose, mice were treated with 10 ug ATC (anhydrotetracycline) via intraperitoneal injection (induced) or with PBS vehicle control (uninduced). Mice were sacrificed at 1, 5, and 24 hours (4 mice per timepoint). Levels TNFα, and IL-1β in the blood was quantified using a custom Luminex kit according to manufacturer's instructions. Results are shown in FIG. 20 and indicate that cytokine levels fall well below those observed in the sepsis control. These data suggest that septic shock and cytokine storm does not occur following treatment of SYN3527 or SYN4023 in tumor bearing mice.

Tumors homogenates (1 hr, 5 hrs, 24 hrs) were analyzed for production of the STING agonist c-di-AMP via liquid chromatography mass spectrometry and CFUs were measured via plating on LB plates. FIG. 20C shows c-diAMP measurements and confirms that c-di-AMP is generated by both SYN3527 and SYN4023 (when induced with ATC) in vivo. FIG. 20D shows CFU counts and demonstrates that SYN3527 colonize and grown in the tumor to a similar extent as the unmodified SYN94, while the ΔDapA strain, SYN4023, shows reduced bacterial numbers over the time period measured (24 hrs). Of note, all blood samples were negative for bacterial growth. Of note, no release of cytokines into the blood stream occurs following treatment with any of the bacterial strains analyzed. Overall, these results indicate that intratumoral treatment of B16F10 tumors and the expression of STING agonist is safe and does not cause a systemic increase in sepsis or cytokine storm related cytokines.

### Example 53. STING Polymorphisms: Building Additional Agonist-Producing Strains to Address Multiple Alleles

Multiple alleles have been described for human STING (hSTING). Different STING agonists activate these alleles with different specificities (see e.g., Yi et al., Single Nucleotide Polymorphisms of Human STING Can Affect Innate Immune Response to Cyclic Dinucleotides; PLOS One October 2013 I Volume 8 I Issue 10 I e77846, the contents of which is herein incorporated by reference in its entirety). STING senses cyclic-di-nucleotides from bacteria and/or an endogenous cyclic GAMP. In addition to production of c-di-AMP, as shown herein for SYN3527, production of additional or alternative agonists could potentially be used to augment stimulation of additional alleles and could provide more flexibility to modulate efficacy of the engineered strain.

A phylogenetic library of putative cGAMP synthases (21 proteins) was identified based on homology to a the known cGAMP synthase (cGAS) DncV. To generate STING agonist strains, cGAS orthologs were cloned into p15 under the control of the Ptet promoter. The cGAS orthologs were then screened to identify alternative enzymes capable of producing cGAMP in vitro.

Briefly, E. coli Nissle strains comprising the heterologous construct encoding one of the cGAS orthologs and a control strain were grown overnight in LB medium. Cultures were diluted 1:10 in M9 minimal media supplemented with 0.5% glucose (w/v) and grown shaking (350 rpm) at 37° C. for 2 hours. Cultures were diluted to an optical density of 1.0 at which time anhydrous tetracycline (ATC) was added to cultures at a concentration of 100 ng/mL to induce expression of cGAS. After 4 hours of induction, samples were removed for LC/MS analysis of cyclic dinucleotide production. Samples were centrifuged for 20 minutes at 5000 RPM to separate cellular and extracellular fractions. Cell pellets were then used to determine intracellular 3′,3′ cGAMP. Concentrations were determined via LC/MS.

Results are shown in FIG. 33A and FIG. 33B. As shown previously, SYN3527 produces high levels of cdAMP. No significant production was observed in other strains tested. However, several significant producers of cGAMP were identified (SYN4251, SYN4240, SYN4241). SYN4251 expresses an ortholog from Verminephrobacter eiseniae (EF01-2 Earthworm symbiont), SYN4240 an ortholog from Kingella denitrificans (ATCC 33394), and SYN4241 an ortholog from Neisseria bacilliformis (ATCC BAA-1200). Sequences are described in Table 55.

TABLE 55
Bacterial cGAMP producing strains

| | | | SEQ ID NO |
|---|---|---|---|
| Strain | | Uniprot | Polypeptide/ |
| Name | Gene Name | ID | Polynucleotide |
| SYN4240 | HMPREF9098_1812 | F0F127 | SEQ ID NO: 1260/SEQ |
| | | | ID NO: 1263 |
| SYN4241 | HMPREF9123_0074 | F2B8M1 | SEQ ID NO: 1261/SEQ |
| | | | ID NO: 1264 |
| SYN4251 | Veis_4659 | A1WRU9 | SEQ ID NO: 1262/SEQ |
| | | | ID NO: 1265 |

### Example 54. Antitumor Efficacy of SYN4023 Treatment for the A20 Tumor Model

Anti-tumor activity of SYN4023 (ptet-DacA and \DapA) was assessed in the A20 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with A20 tumors (1e6 cells/mouse in PBS) SC into the right flank of each animal on Day −14. On Day −3 tumor growth was monitored; when the tumors reached ˜40-80 mm³ mice (Day 0) were randomized into experimental groups (N=12 per group) for intratumor dosing as shown in Table 56.

TABLE 56
Experimental groups

| Group No | | |
|---|---|---|
| (n = 12) | Treatment | Dosing Schedule |
| 1 | Saline | 40 uL saline day 1 and 4 |
| 2 | SYN4023 (ptet-DacA; | [1e8 CFU] in 40 uL day |
| | ΔDapA) | 1 and 4 |

Animals were dosed with SYN4023 bacteria or saline I.T. on day 1 and day 4. Four hours before each bacterial dose, mice were pretreated with 10 ug ATC (anhydrotetracycline) via intraperitoneal injection. Tumor volume was measured, and animal health was assessed 3x/week. Results are shown in FIG. 21 and indicate that treatment of A20 tumors with SYN4023 results in tumor control and rejection. These data demonstrate the SYN4023 treatment is efficacious with only two doses in the A20 tumors model.

### Example 55. Augmentation of Immune Stimulator Efficacy with DAP Auxotroph STING Agonist Producing Strain

Agonistic antibodies triggering immune stimulation via OX40, 41BB and GITR have been shown to promote antitumor immunity via the expansion and proliferation of cytotoxic CD8+ and helper CD4+ T cells (Sanmamed, M. F., et al. (2015). “Agonists of Co-stimulation in Cancer Immunotherapy Directed Against CD137, OX40, GITR, CD27, CD28, and ICOS.” Semin Oncol 42(4): 640-655, the contents of which is herein incorporated by reference it's entirety), and thus we sought to evaluate the combined efficacy of these treatments with our STING agonist producing bacterial strains.

To determine whether SYN4023 (E. coli Nissle comprising plasmid-based, tetracycline-inducible p15a Ptet-DacA from Listeria monocytogenes and ΔDapA) can augment efficacy of aOX40, a41BB, and aGITR, tumor growth was monitored in a B16F10 tumor model.

To produce cells for the study, overnight cultures were used to inoculate 500 mL LB medium with antibiotic. The strains were incubated with shaking at 37 C until the cultures reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with B16F10 tumors (2e5 cells/mouse in PBS) SC into the right flank of each animal on Day −9. On Day −3 tumor growth was monitored; when the tumors reached ˜40-80 mice (Day 0) were randomized into experimental groups (N=10 per group) for intratumor dosing as shown in Table 57.

TABLE 57
Experimental groups

| Group No | Intratumoral | Systemic |
|---|---|---|
| (n = 10) | Treatment | Treatment |
| 1 | PBS | PBS |
| 2 | PBS | αOX40 |
| 3 | PBS | α41BB |
| 4 | PBS | αGITR |
| 5 | SYN4023 (DAP-STING) [1e8] | PBS |
| 6 | SYN4023 (DAP-STING) [1e8] | αOX40 |
| 7 | SYN4023 (DAP-STING) [1e8] | α41BB |
| 8 | SYN4023 (DAP-STING) [1e8] | αGITR |

Animals were dosed with SYN4023 bacteria I.T. and appropriate antibody I.P. twice a week for the duration of the study. Antibody treatments (I.P.) were as follows: anti-OX-40 (anti-OX-40 at 100 μg per dose), anti-GITR (DTA-1 at 350 μg per dose), and a4-1BB (LOB12.3 at 350 μg per dose). Four hours before each bacterial dose, mice were treated with 10 ug ATC (anhydrotetracycline) via intraperitoneal injection. Bacterial dosing was stopped for complete responders. Tumor growth was measured on days 3, 6, 9 and 13 as shown in FIG. 22A (median volume) and FIG. 22B, FIG. 22C, FIG. 22D, FIG. 22E, FIG. 22F, FIG. 22G, and FIG. 22H (individual mice). Results indicate that co-administration of SYN4023 with various agonist antibodies results in superior control of tumor growth and tumor rejection compared to agonist antibodies alone. These data, as well as those described above, demonstrate the ability of STING-agonist producing to improve the efficacy of both immune checkpoint and immune agonist therapies.

### Example 56. In Vivo Promoter Studies in the B16F10 Tumor Model

As a means of control over immunological payload delivery, the function and utility of several inducible promoter systems was evaluated. The activity of low oxygen inducible FNR, cumate-inducible, and salicylate-inducible promoters were assessed in the tumor microenvironment in vivo.

To evaluate expression of various inducible promoters within tumors following induction, GFP reporter constructs were generated driven by the various promoters to be tested. RFP driven by a constitutive promoter was used to identify and isolate the bacterial cells within the tumors.

B16F10 (implanted at 2e5) were implanted as described herein and allowed to reach a tumor size of −100 mm³ before dosing. On day 1 of the study, mice were randomized into groups (8 mice per group; 4 mice per time point) and injected with the inducible-GFP strains as listed in Table 58. Inducers were administered I.P. at 48 hrs post bacteria injection (Day 3).

TABLE 58
Experimental groups

| Group | Strain | Inducer |
|---|---|---|
| 1 | SYN4360 (Tet-GFP:Con-RFP) [0.5e6] | aTC (10 ug) |
| 2 | SYN4359 (fnr-GFP:Con-RFP) [0.5e6] | none |
| 3 | SYN4352 (asp-GFP:Con-RFP) [0.5e6] | Sodium salicylate (150 |
| | | uL of 100 mM in PBS) |
| 4 | SYN4352 (asp-GFP:Con-RFP) [0.5e6] | none |
| 5 | SYN4353 (cum-GFP:Con-RFP) [0.5e6] | water-soluble Cumate |
| | | (6 mg in 200 uL PBS) |
| 6 | SYN4353 (cum-GFP:Con-RFP) [0.5e6] | none |
| 7 | No injection control | none |

At 1 and 16 hrs post inducer injection (Day 3 and Day 4), tumors were crushed in 40 uM cell strainer and spun at 700 g for 5 min. Supernatants were transferred to a new tube and spun at 4,000 rpm for 10 min. Pellets were resuspended in 500 uL PBS and 200 uL was transferred to 96-well plate for flow cytometric analysis. Results are shown in FIG. 24 and FIG. 25. FIG. 24 shows the percentage of RFP positive (bacterial cells, no tumor cells) which are GFP-positive (i.e., in which the promoter is active). Note, the percentage of bacterial cells in which the three promoters are active was the same at 1 and 16 hrs. FIG. 25 depicts the mean fluorescence intensity (MFI) measuring overall reporter gene expression levels. With exception of the tet-inducible construct, which shows higher expression levels at 16 hours, these expression levels also remain the same over the two timepoints. Taken together, these results indicate that all three promoters tested are capable of driving reporter gene expression, a proxy for an immunological payload, in vivo in the tumor.

### Example 57. Administration of WT STING Strain Results in Long-Term Immunological Memory

The ability of the complete regressions elicited by SYN3527 (WT Tet-STING) to result in long lasting immunological memory was assessed in the A20 tumor model.

Animals (n=4) with complete regression from SYN3527 treatment in the SYN3527 Dose Response Study (Example 52, FIG. 65) were used and compared to naïve age-matched mice.

Naïve control mice were implanted with A20 tumors (2e5 cells/mouse in PBS) SC into the right flank of each animal on Day −14. Mice previously treated with SYN3527 (and having shown complete regression) were implanted with A20 tumors (2e5 cells/mouse in PBS) SC into the left flank, opposite flank from original tumor, on day 60 after the original treatment.

TABLE 59
Experimental groups

| Group No | | |
|---|---|---|
| (n = 4) | Mice | Treatment |
| 1 | Animals with complete | Implant cells on left flank- |
| | regressions from SYN3527 | opposite flank from original |
| | treatment in SYN3527 | tumor in mice previously |
| | Dose Response Study | treated with SYN3527 |
| 2 | Naïve Age-matched controls | Right flank |

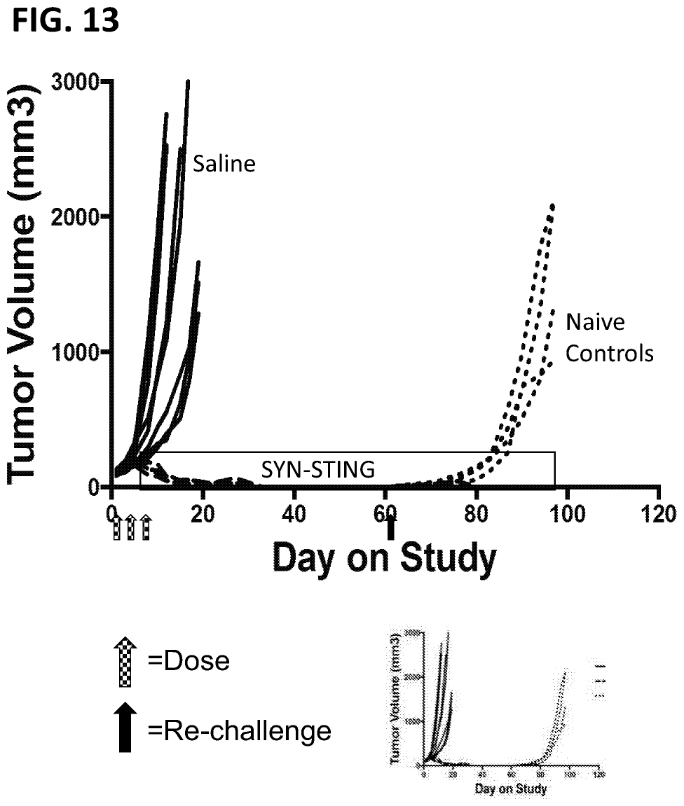

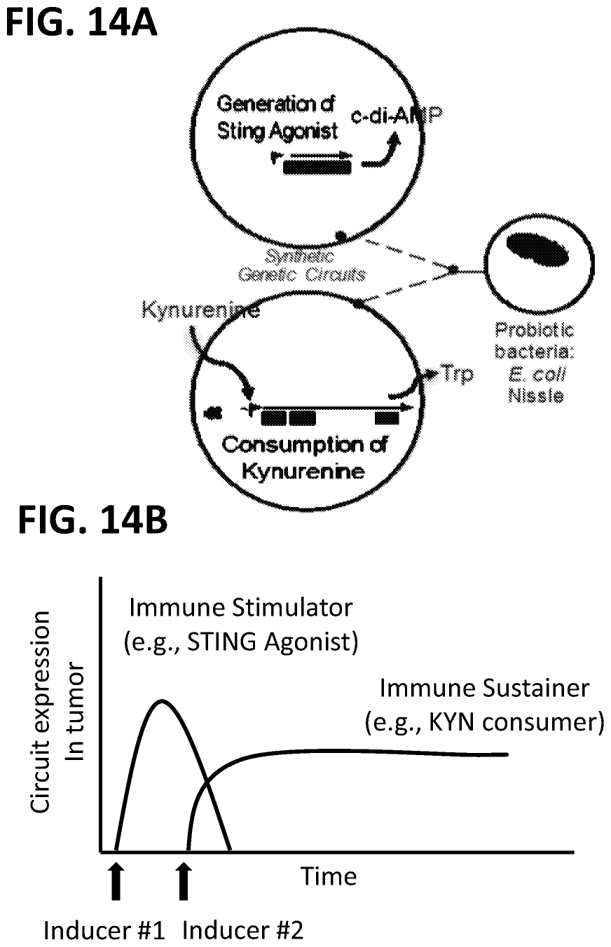

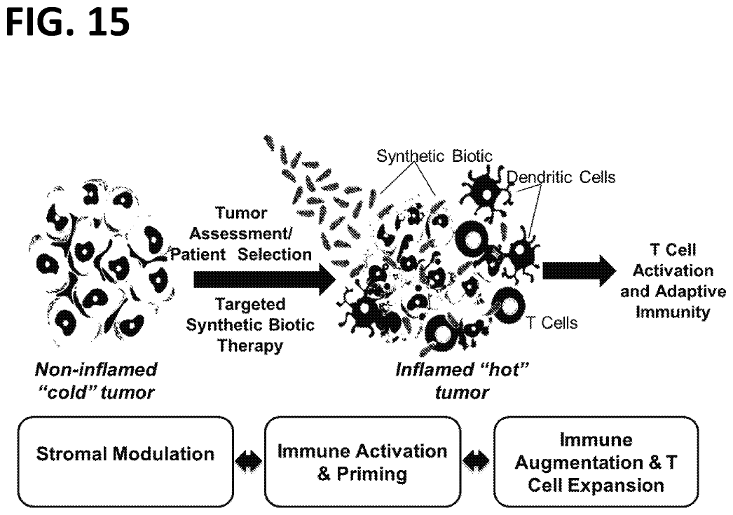

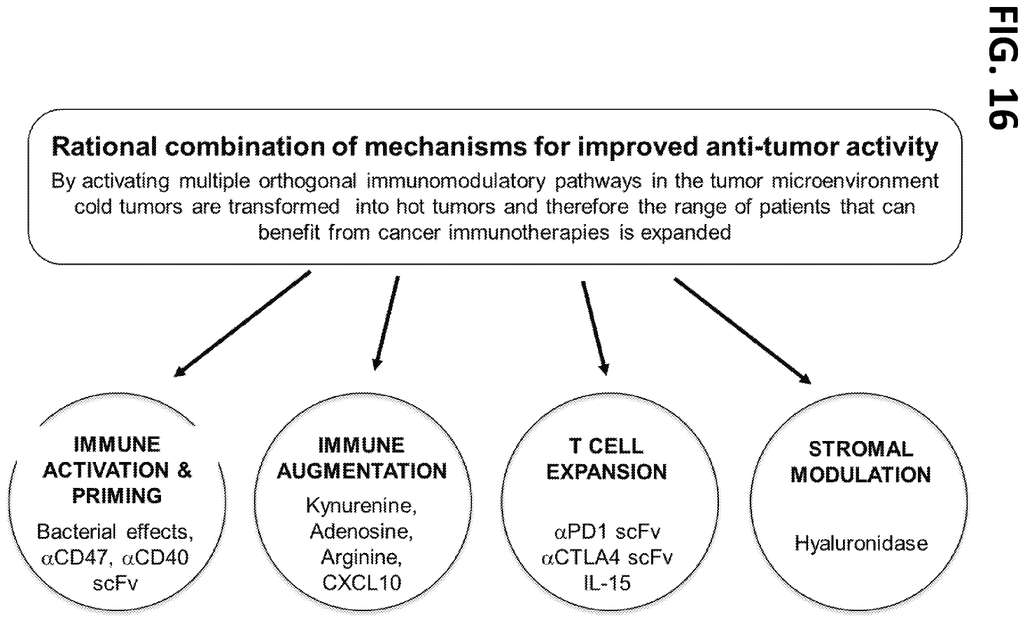

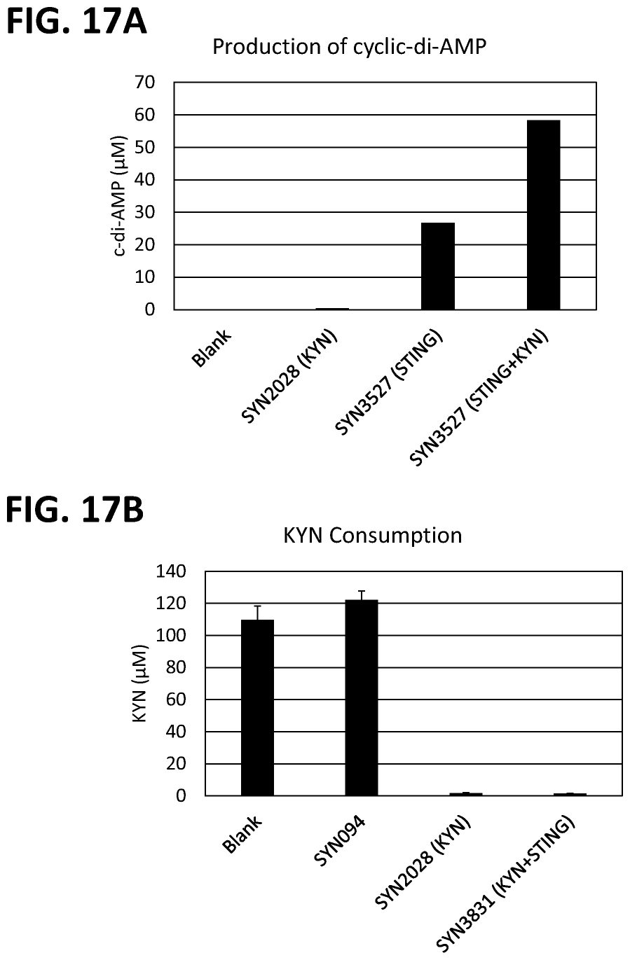

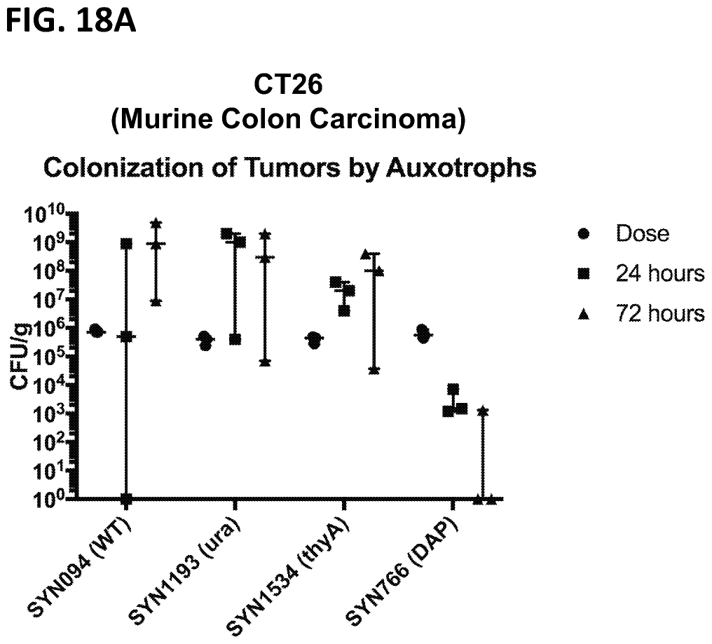

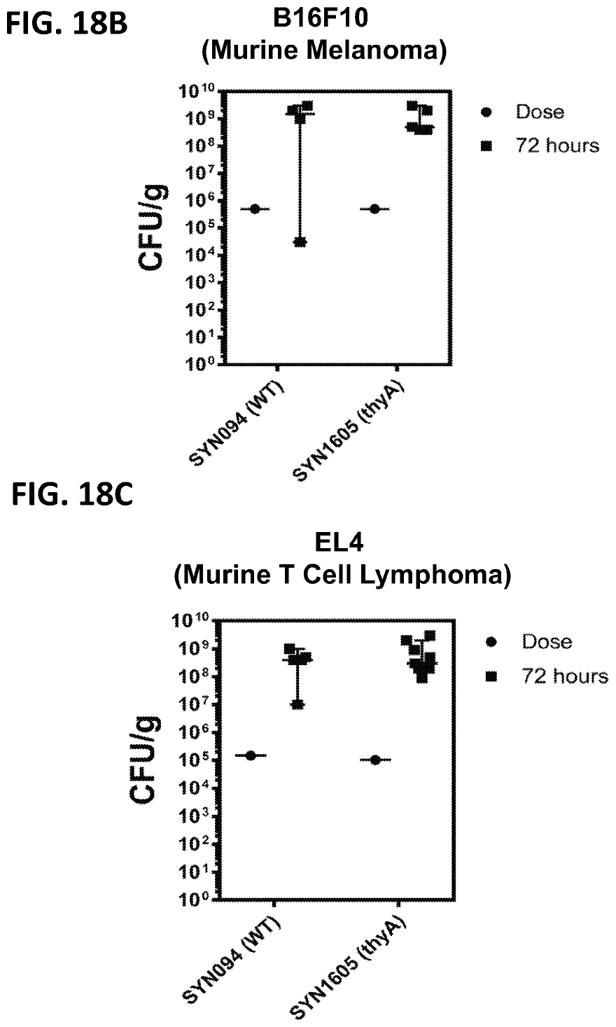

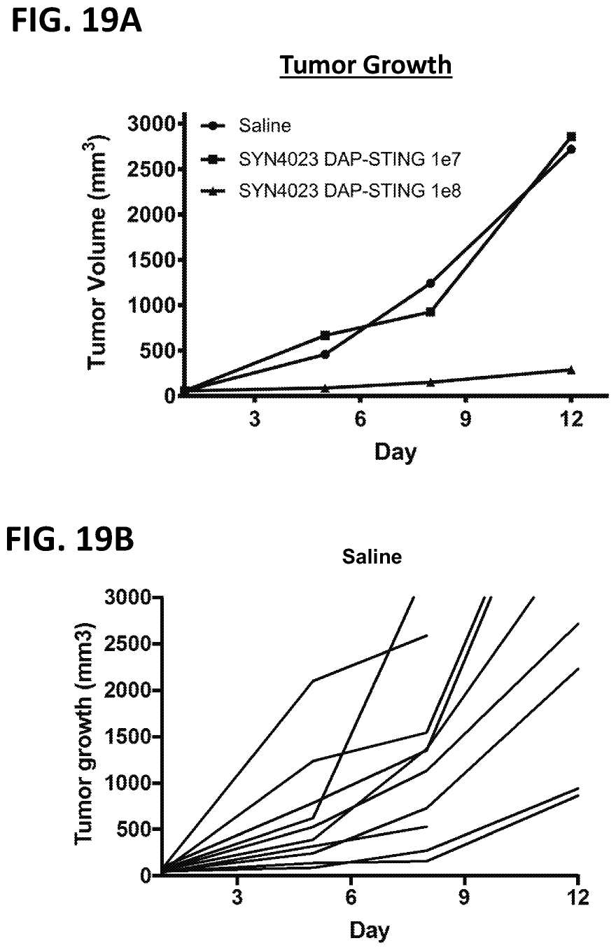

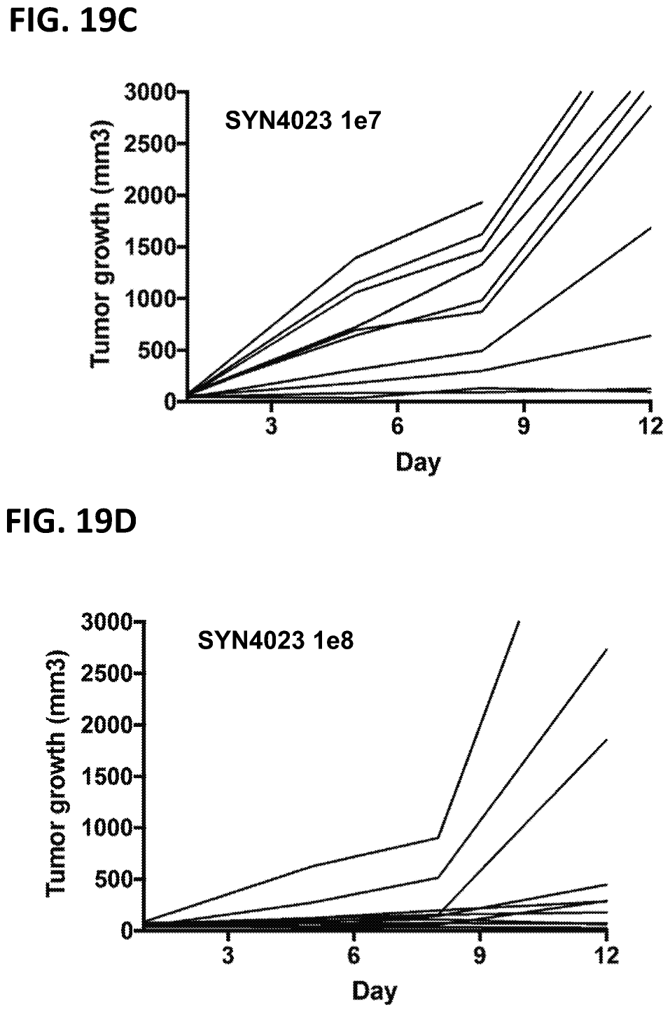

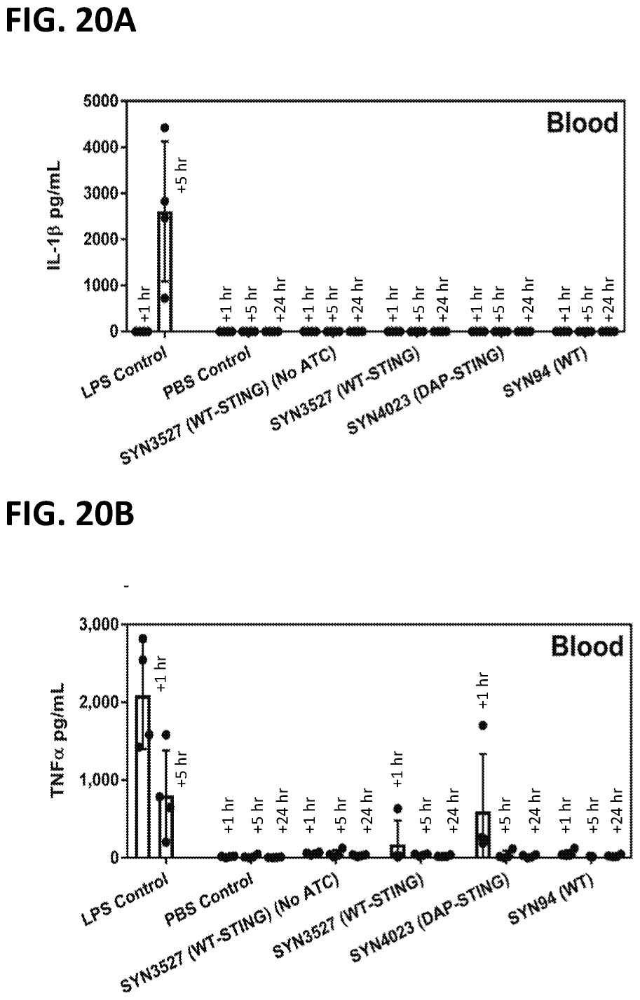

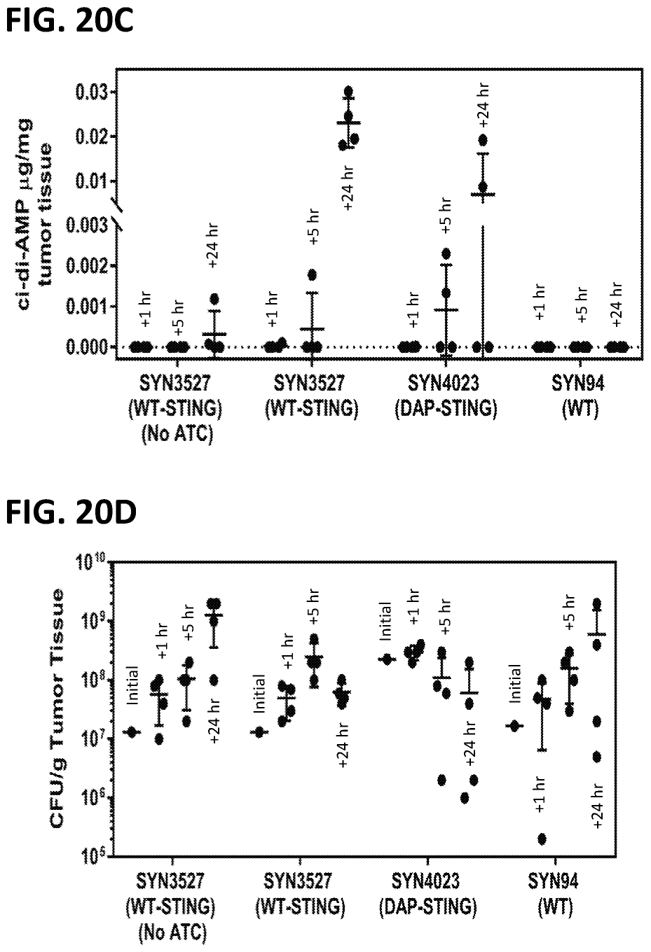

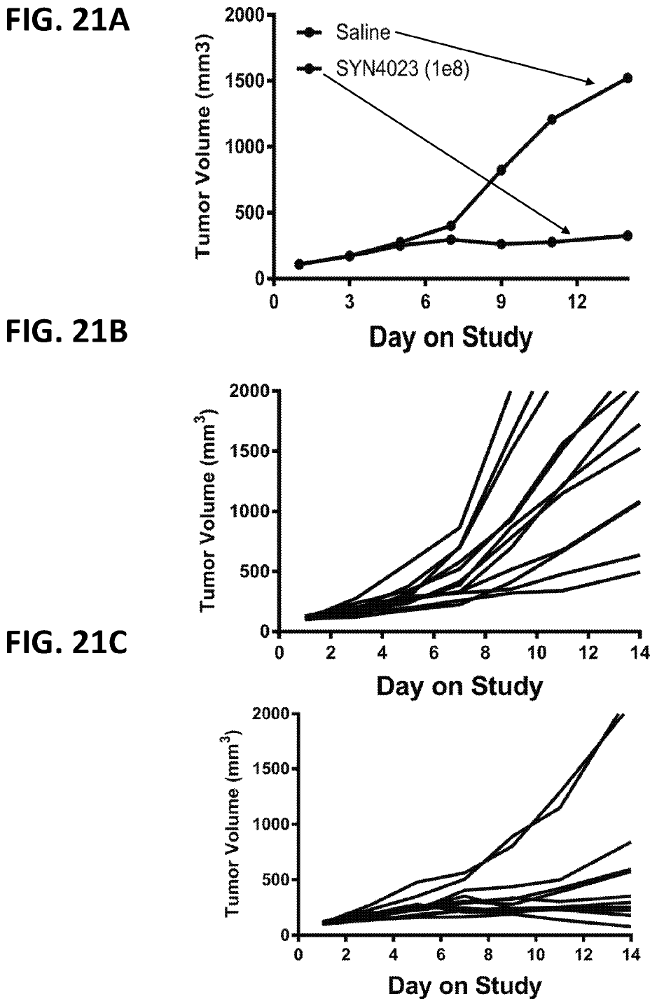

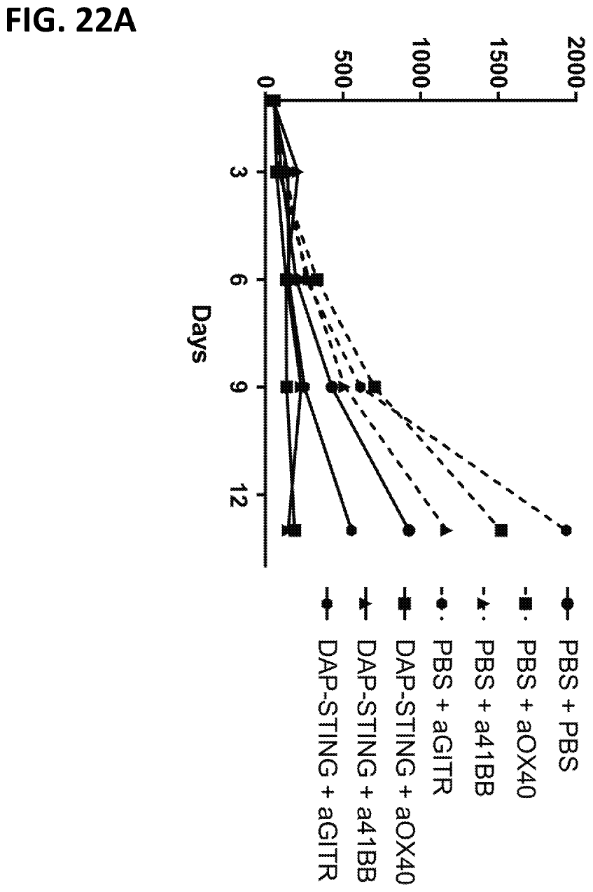

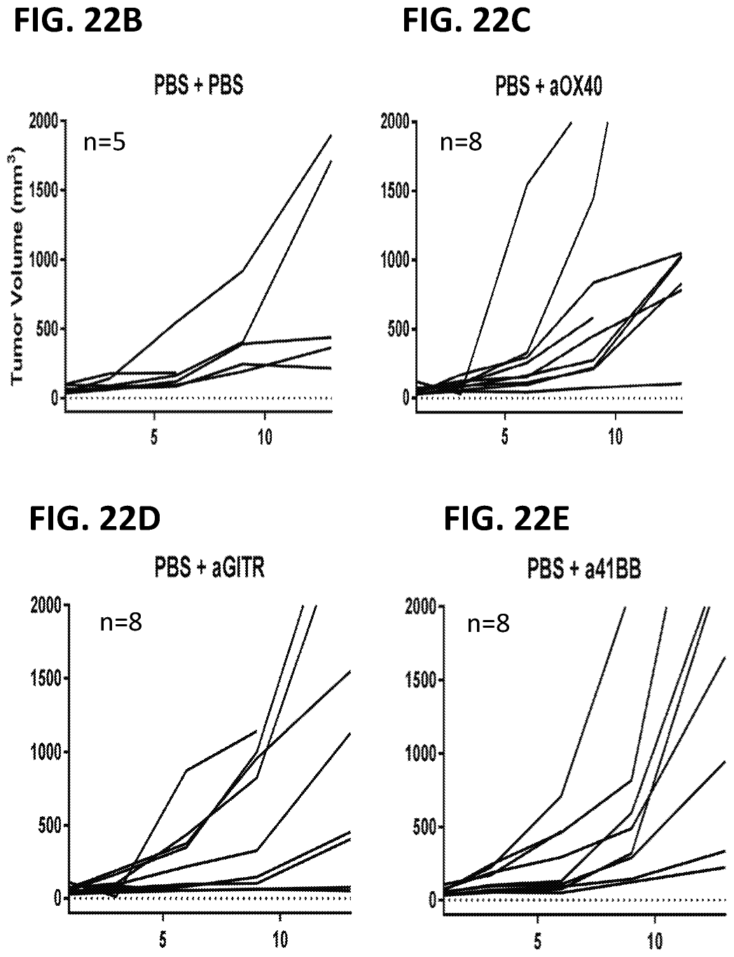

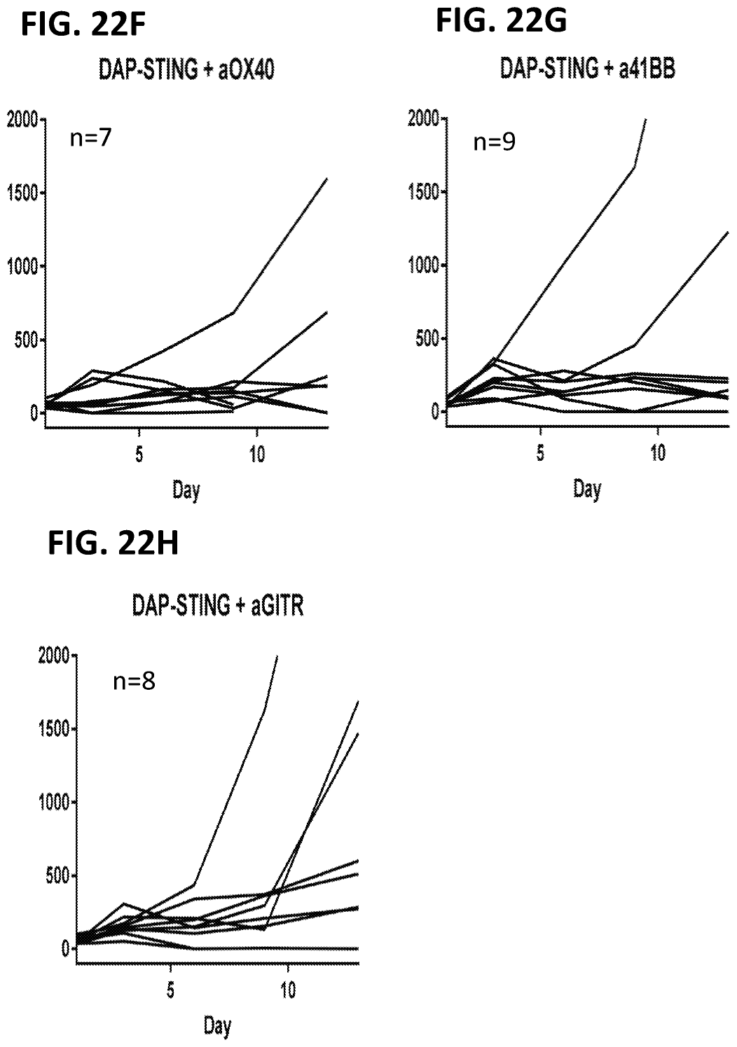

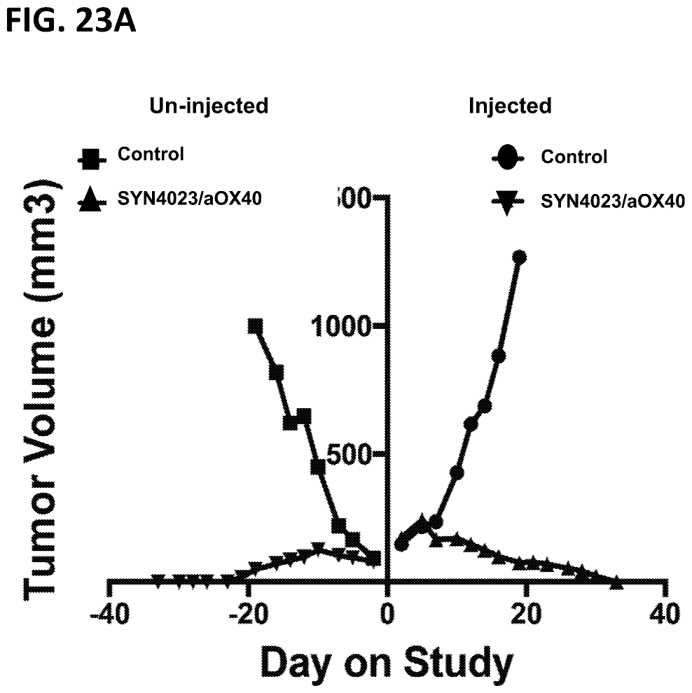

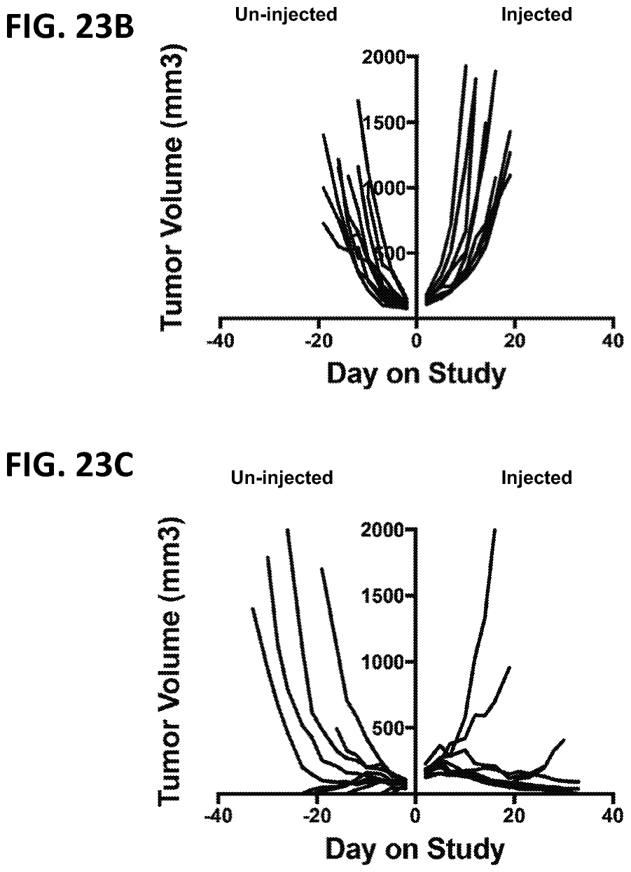

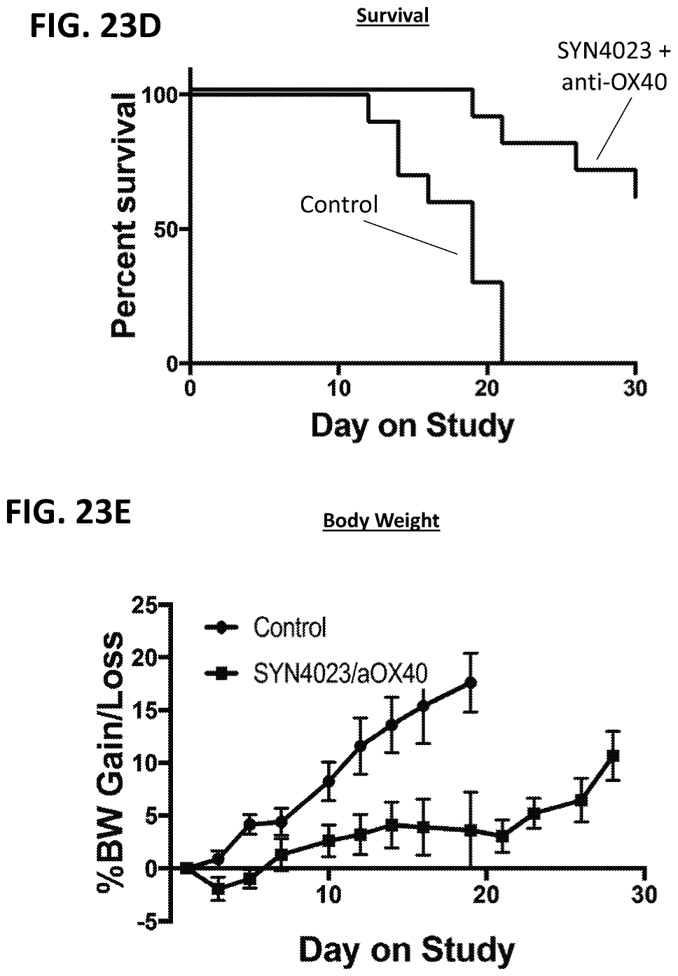

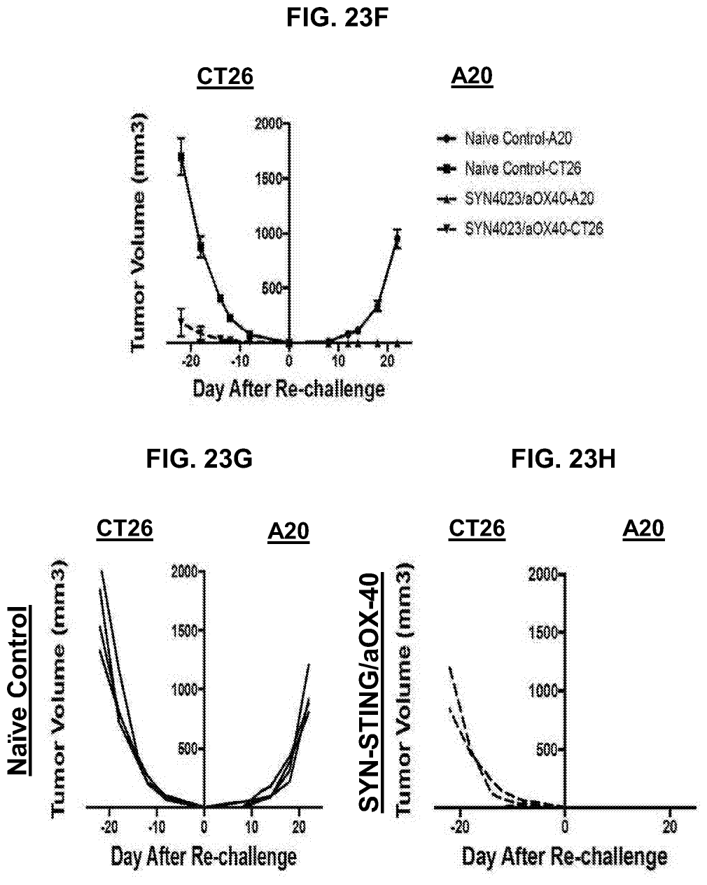

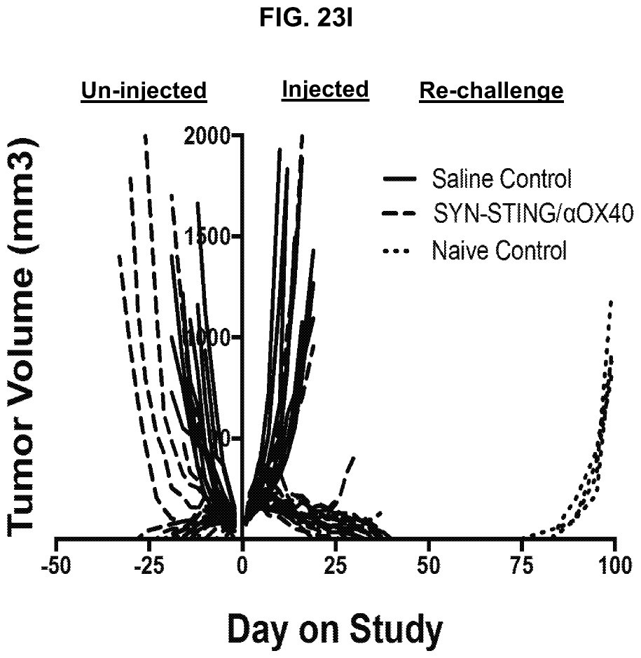

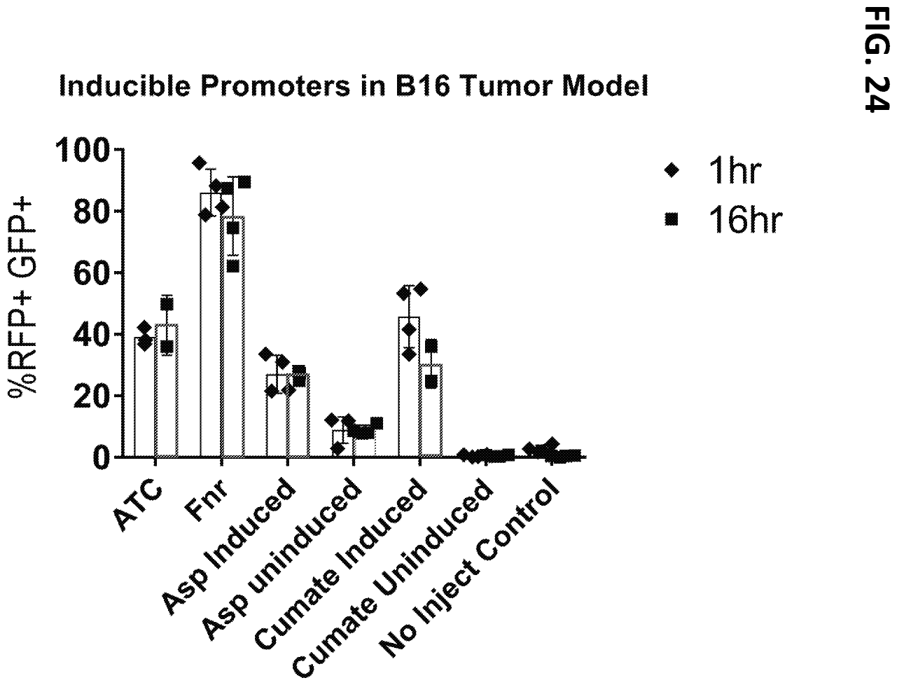

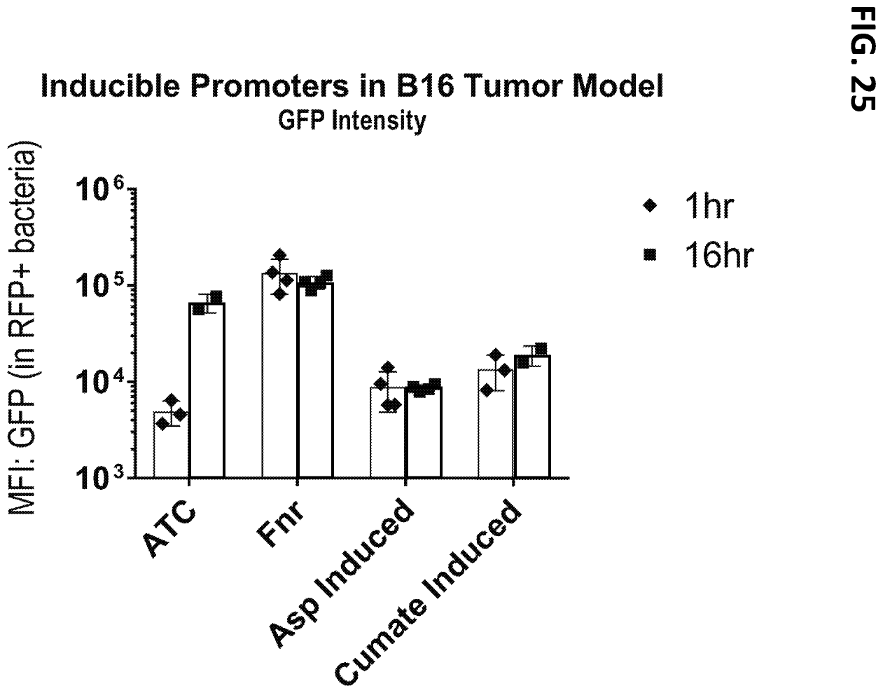

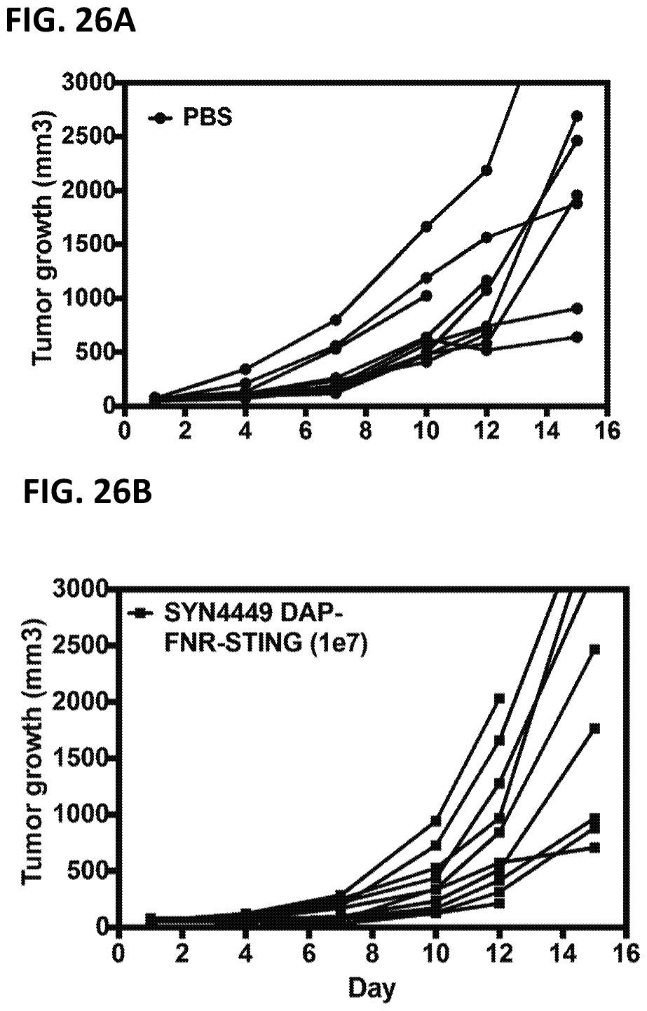

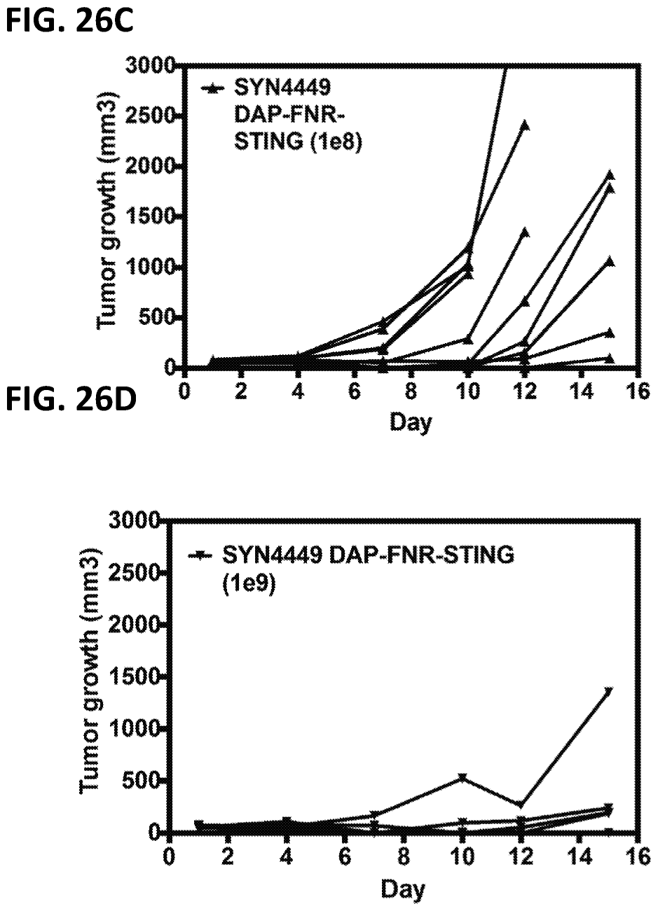

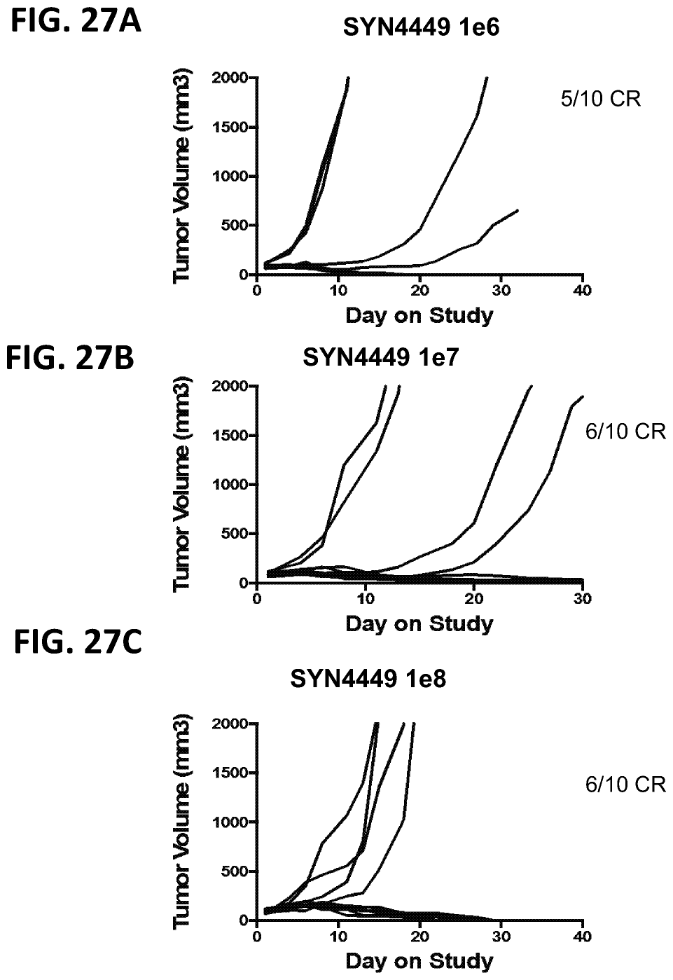

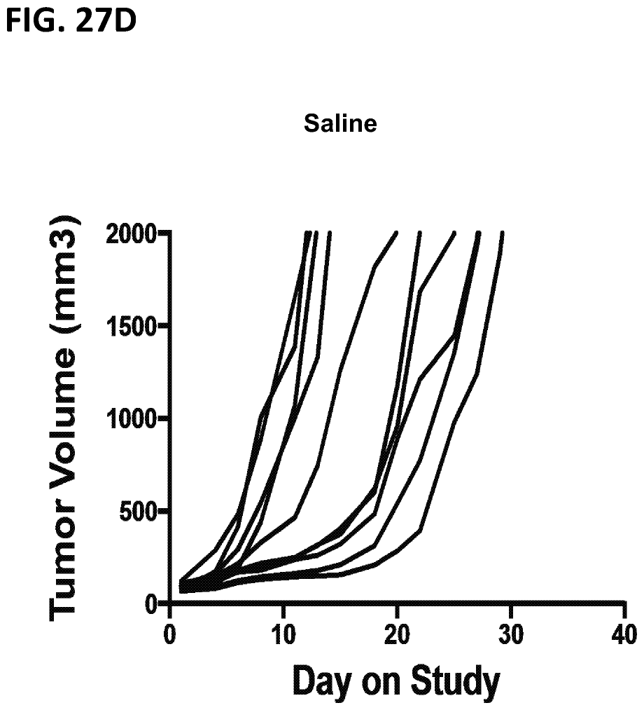

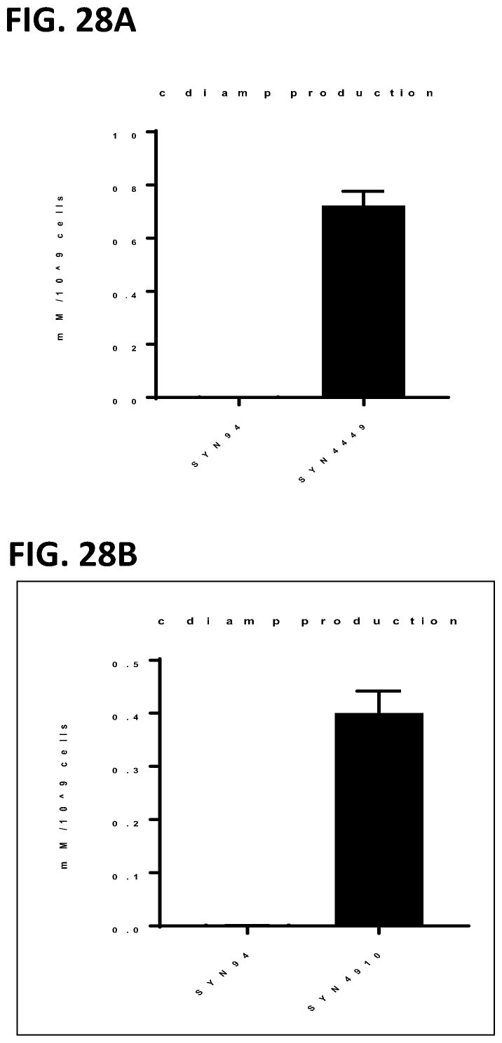

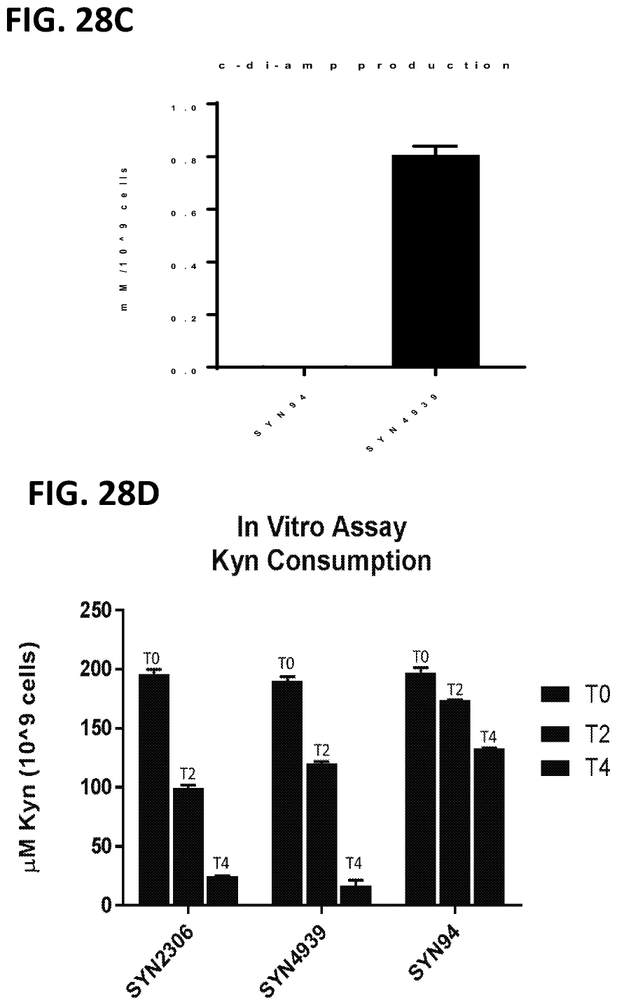

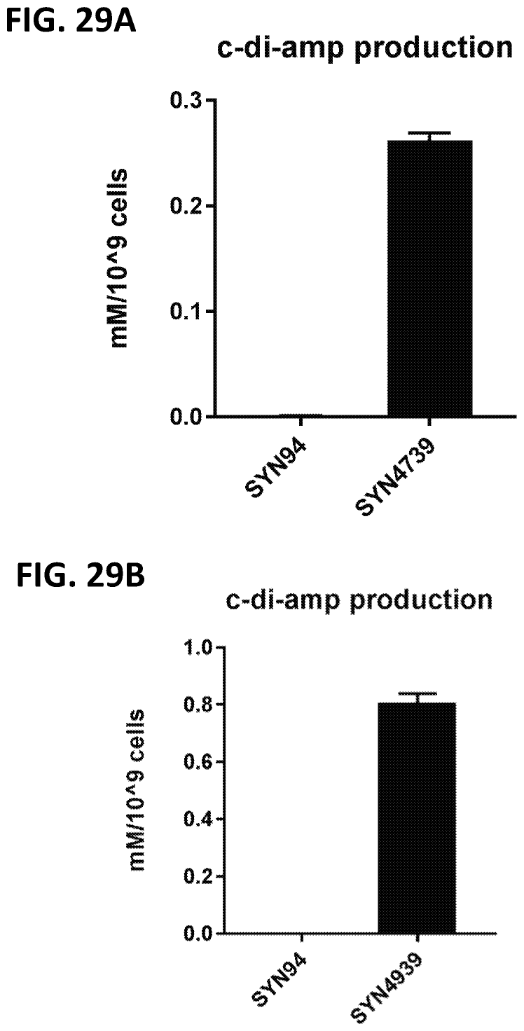

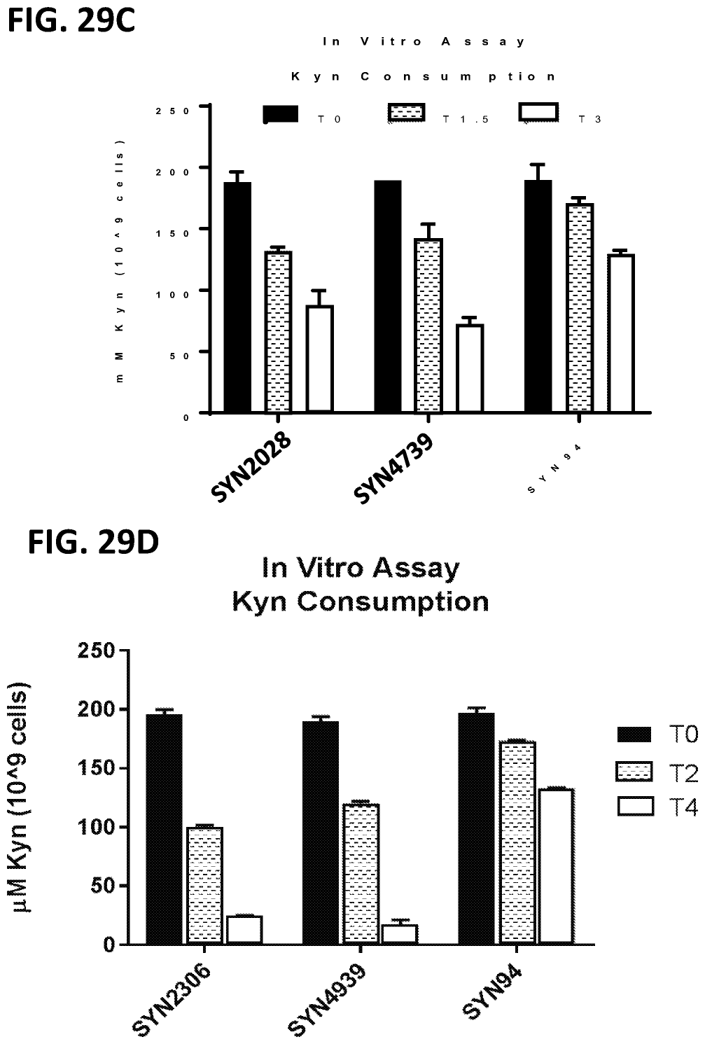

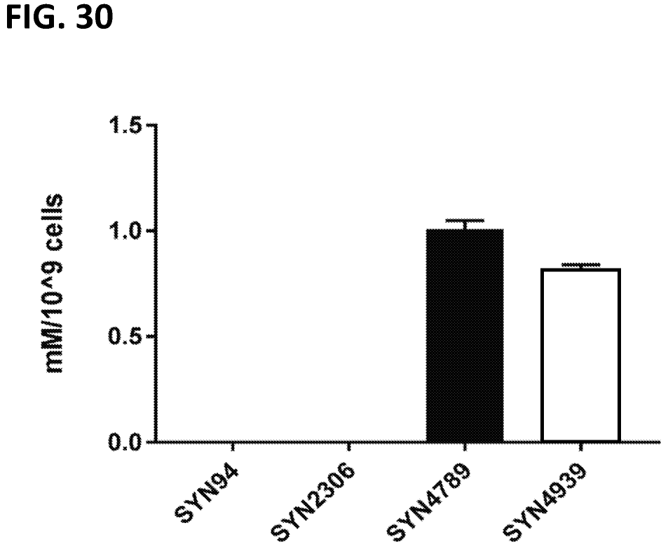

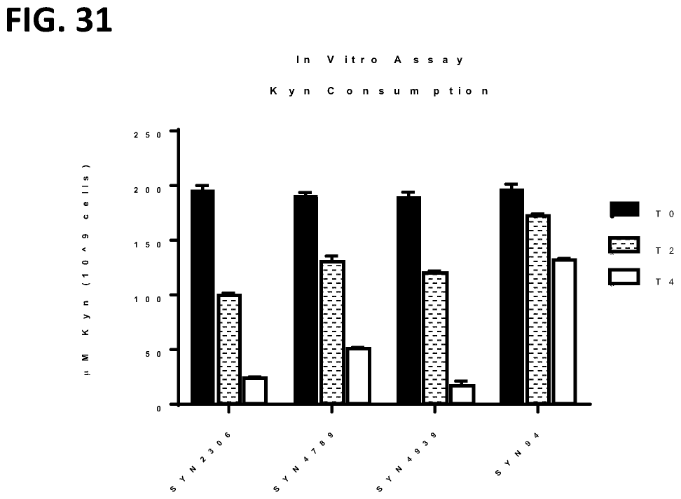

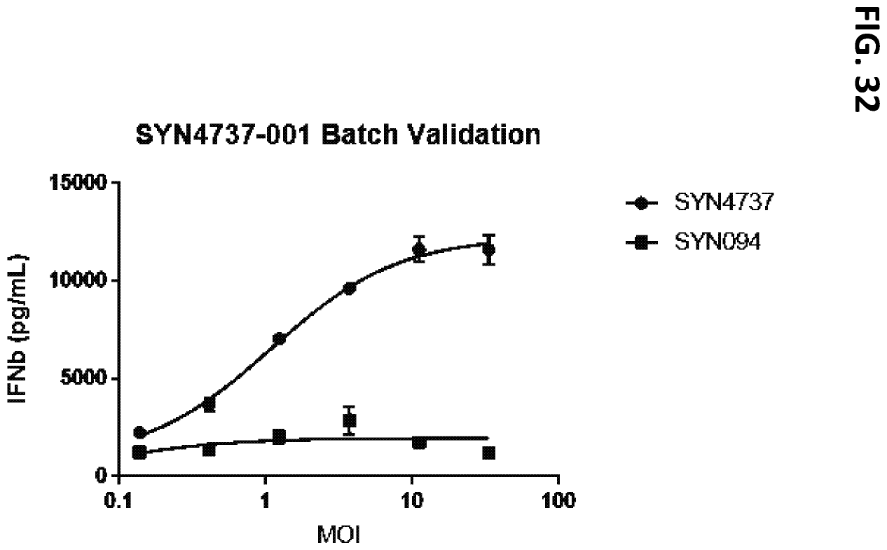

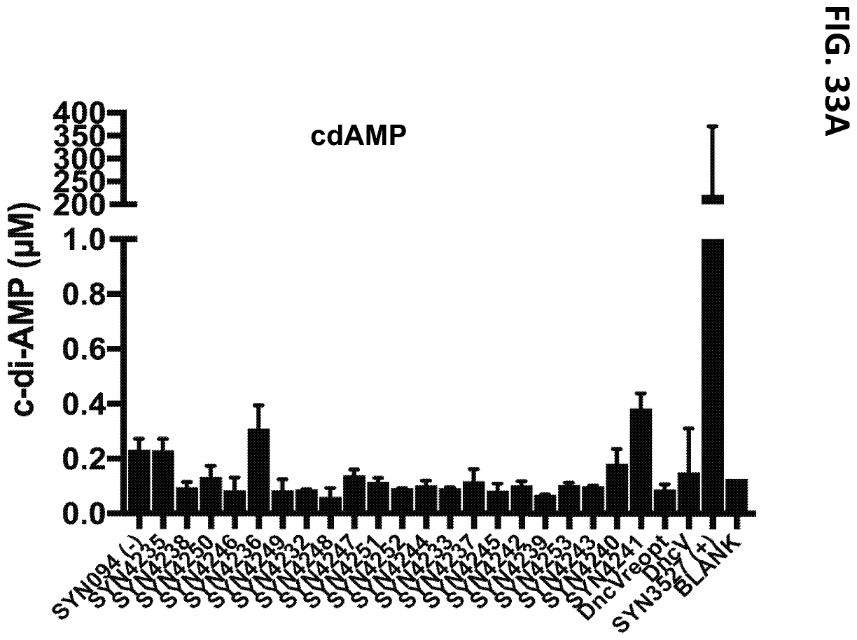

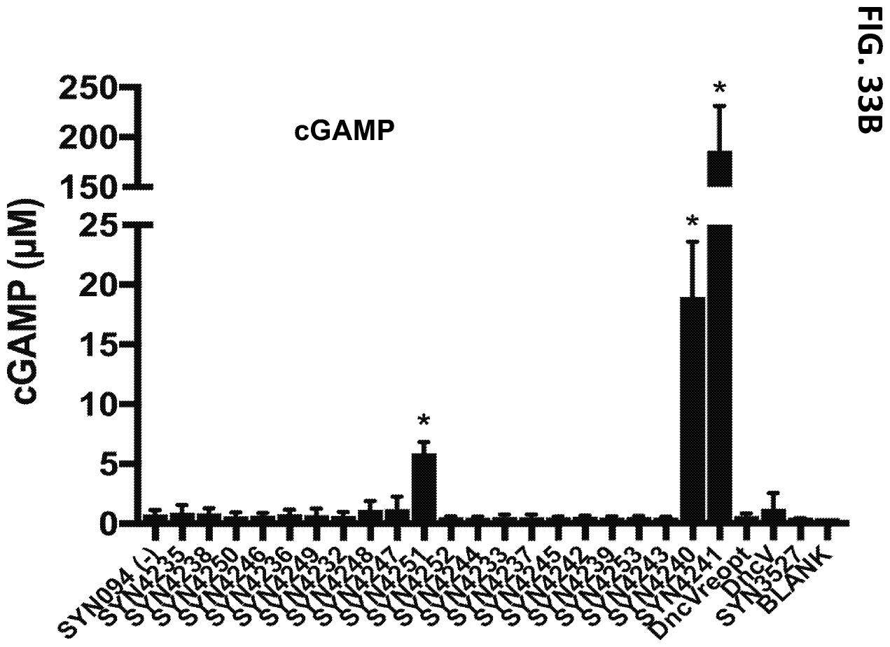

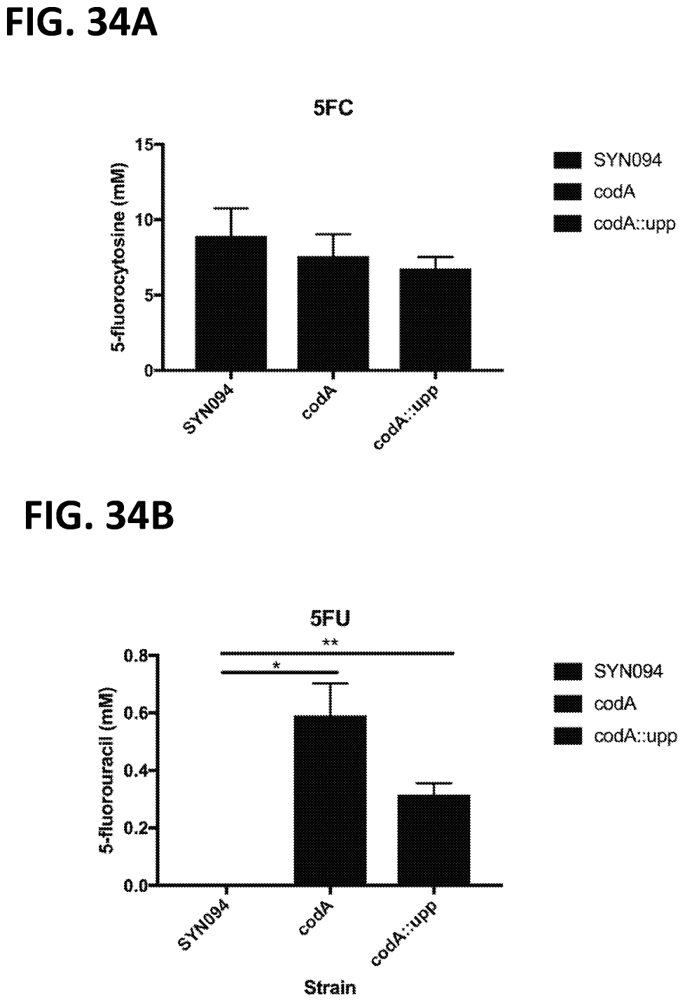

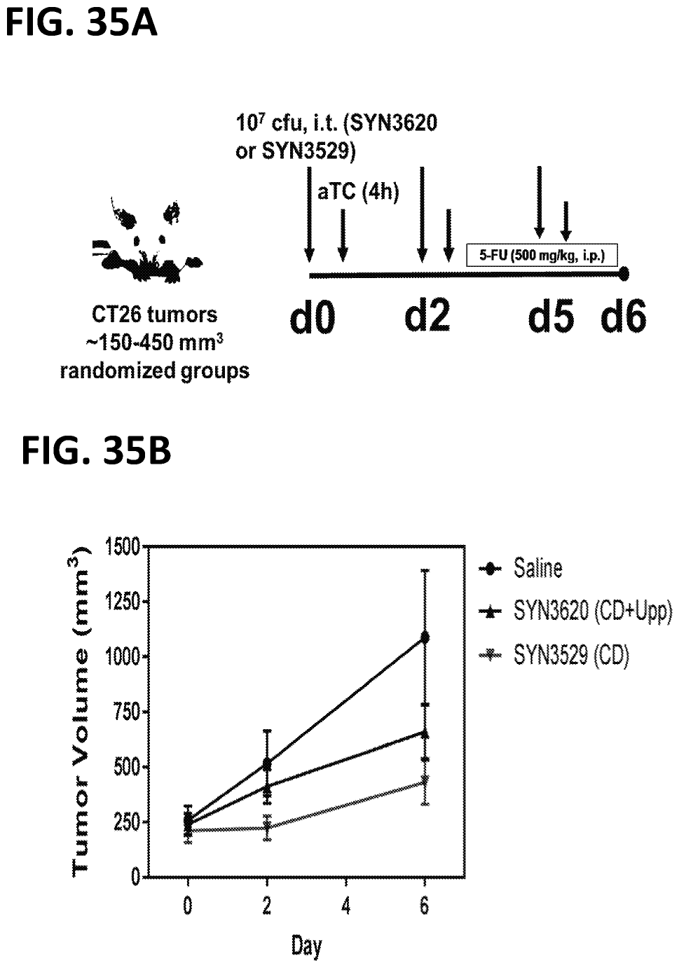

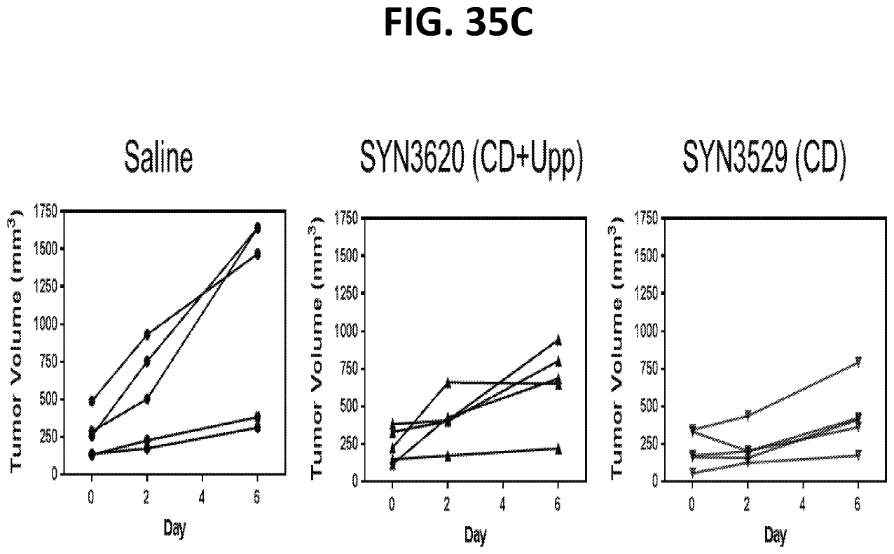

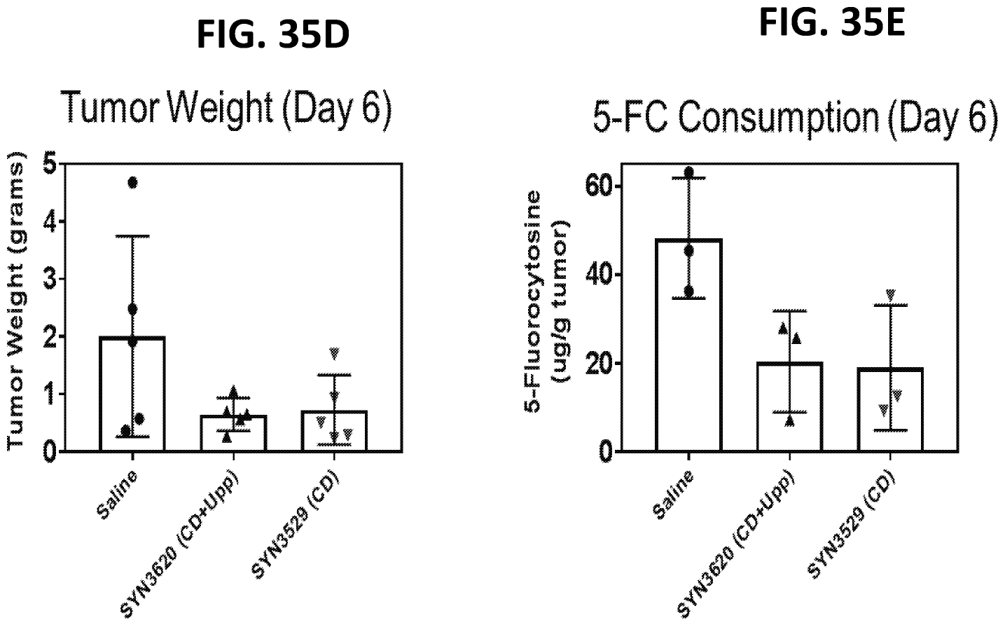

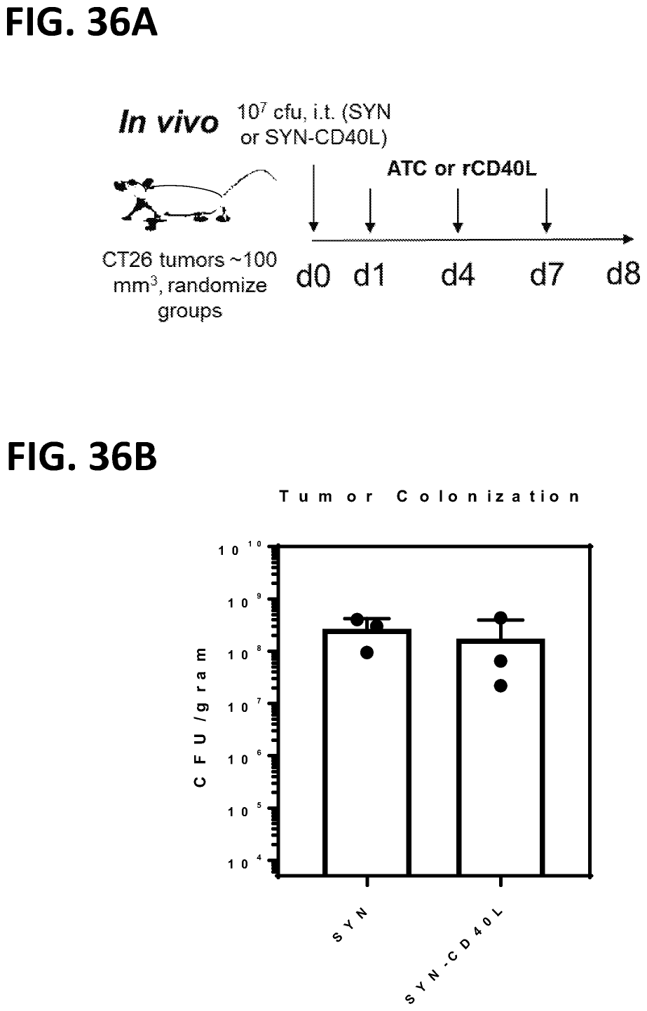

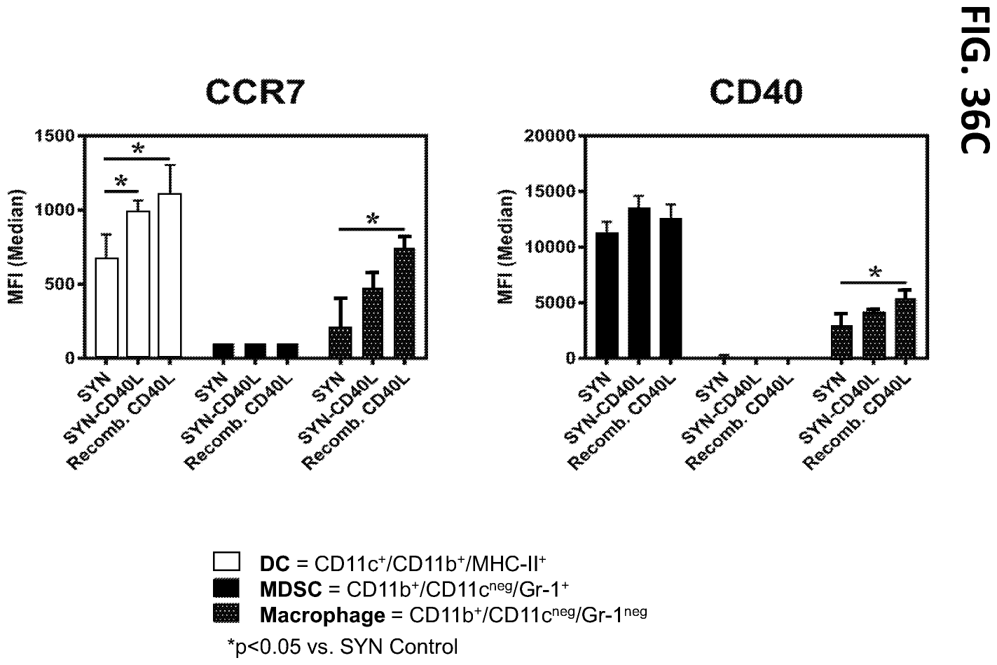

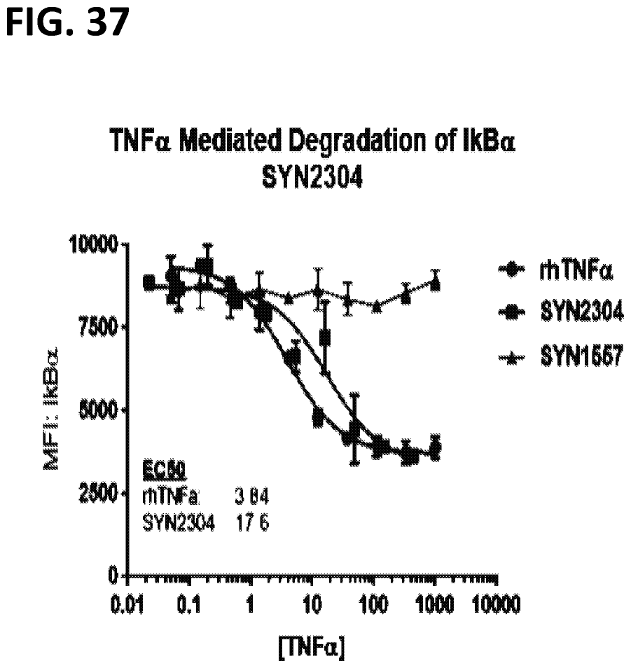

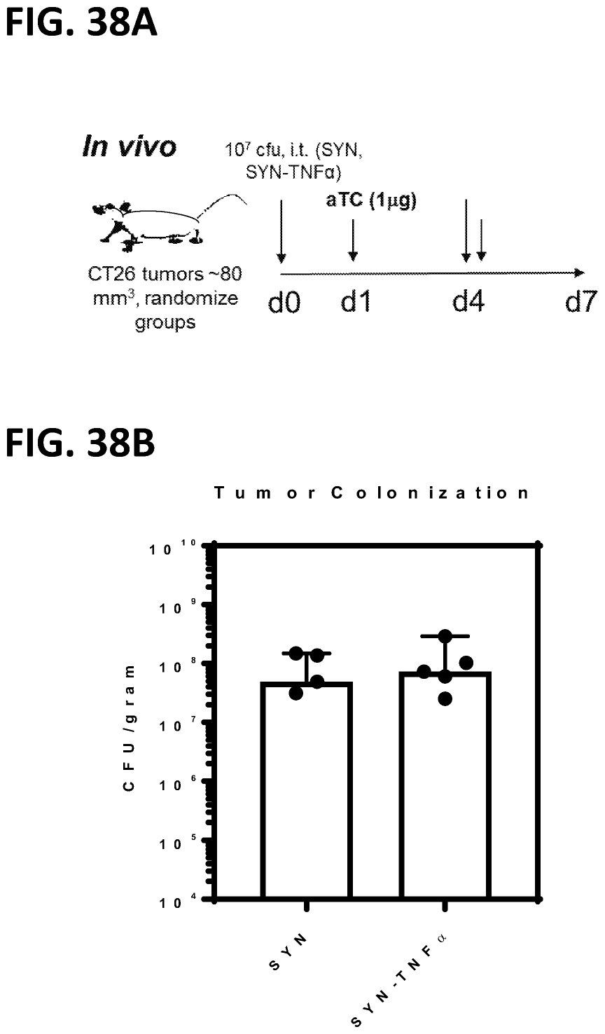

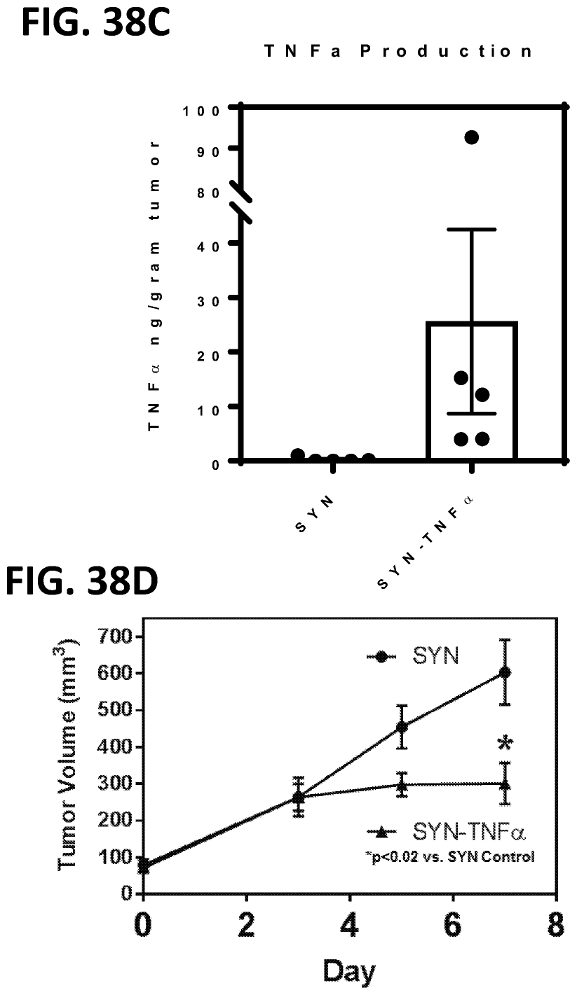

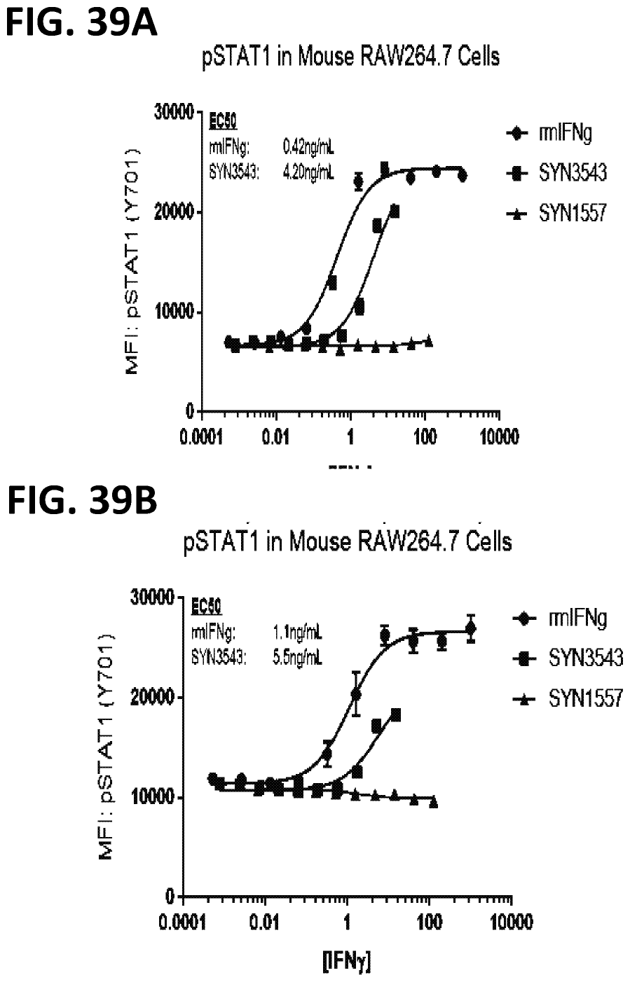

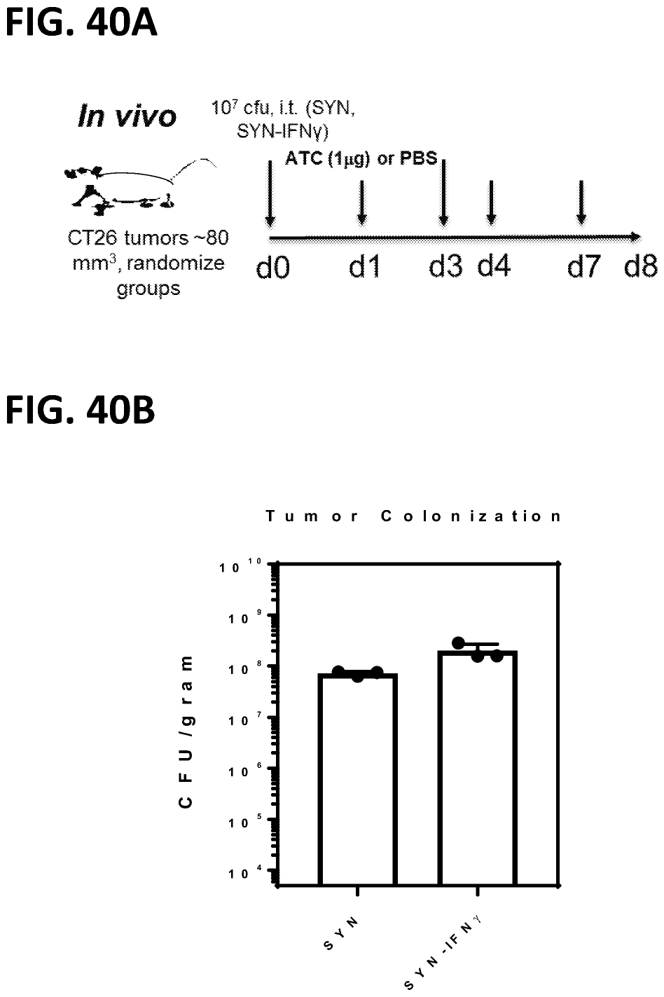

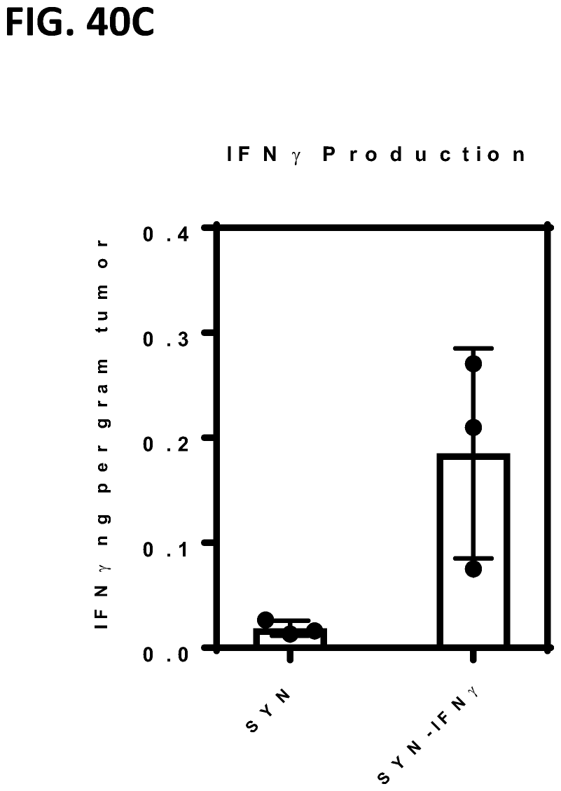

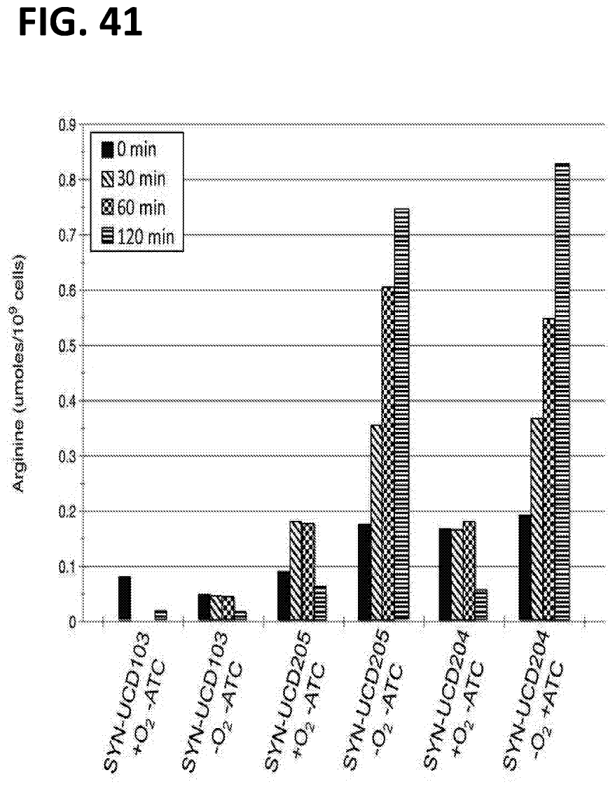

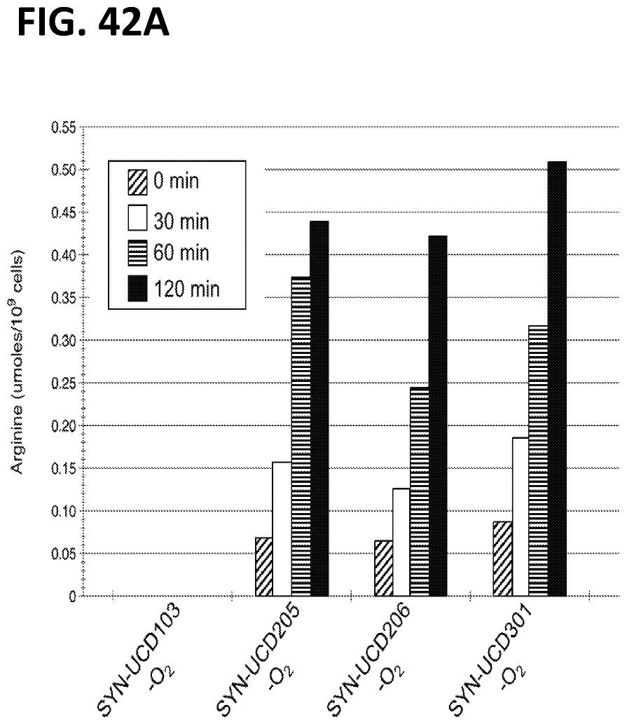

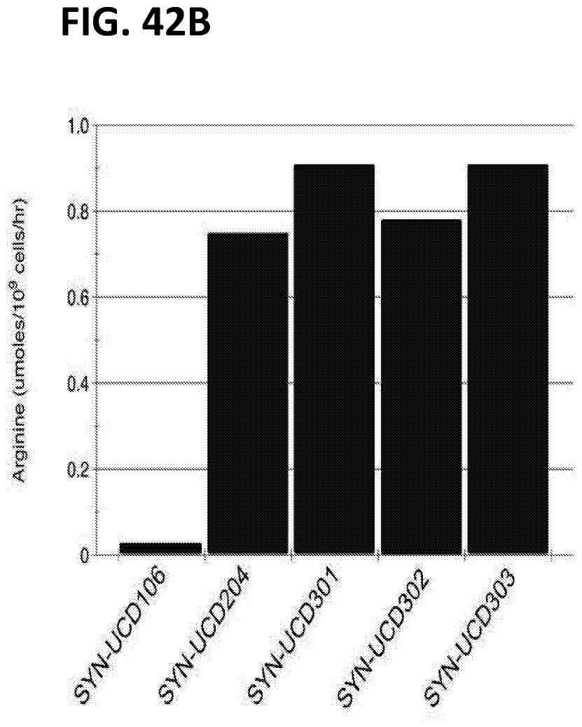

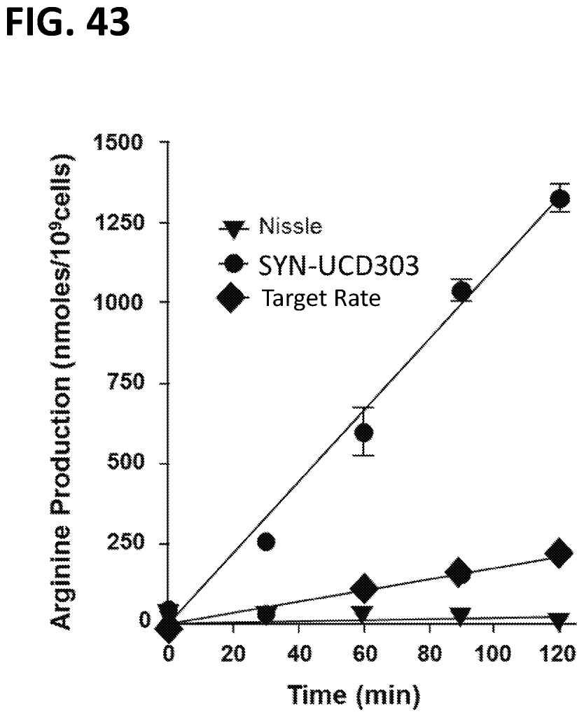

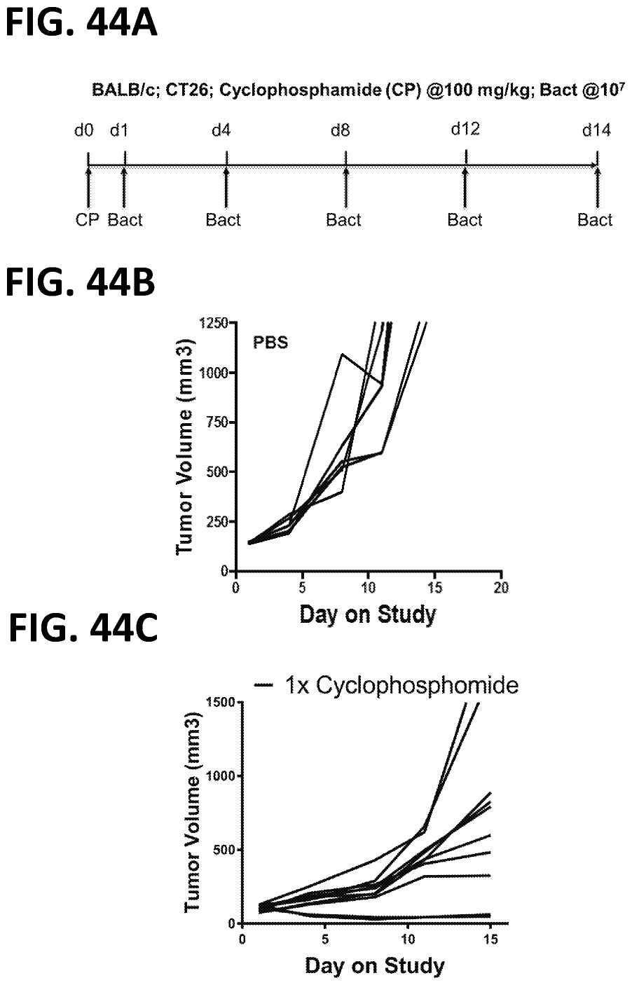

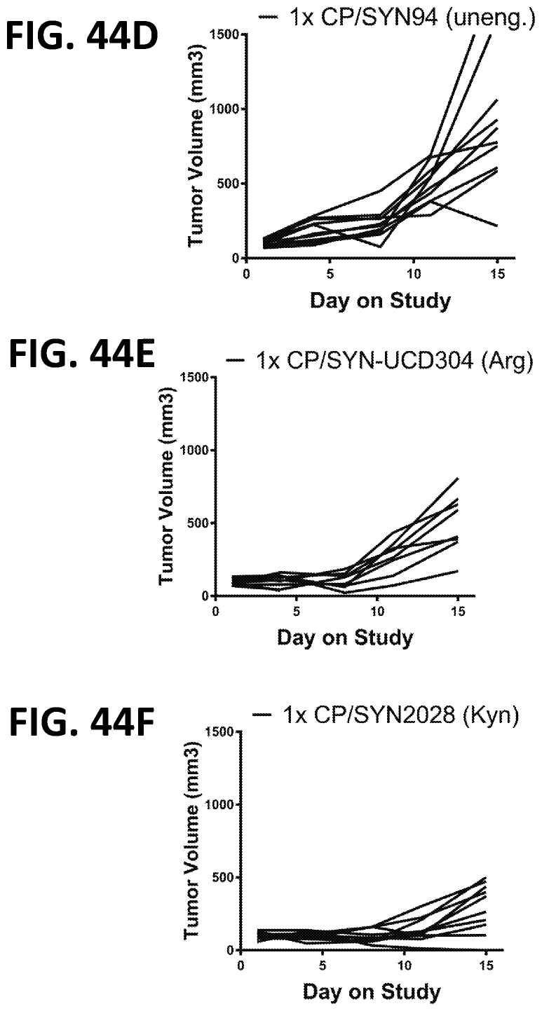

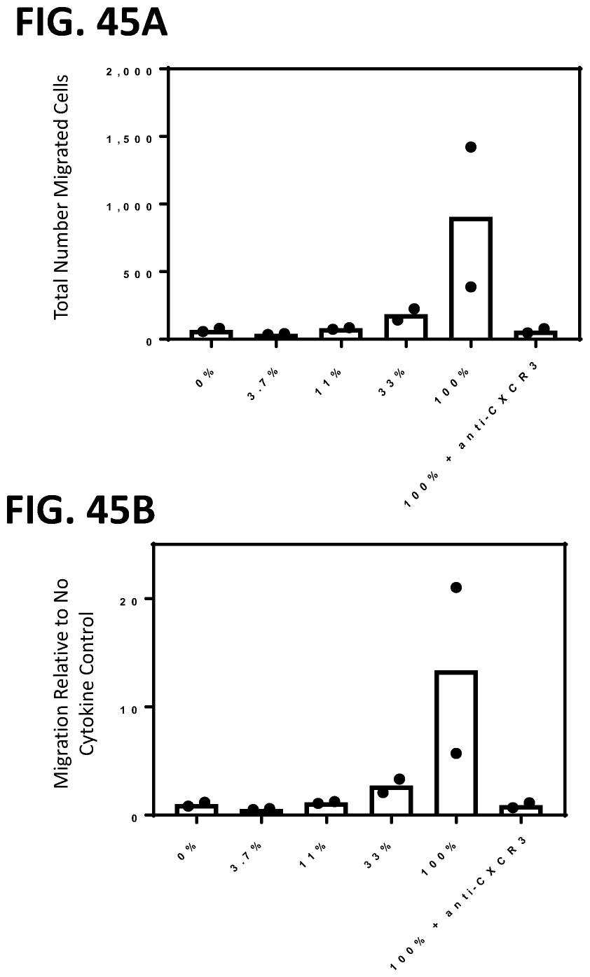

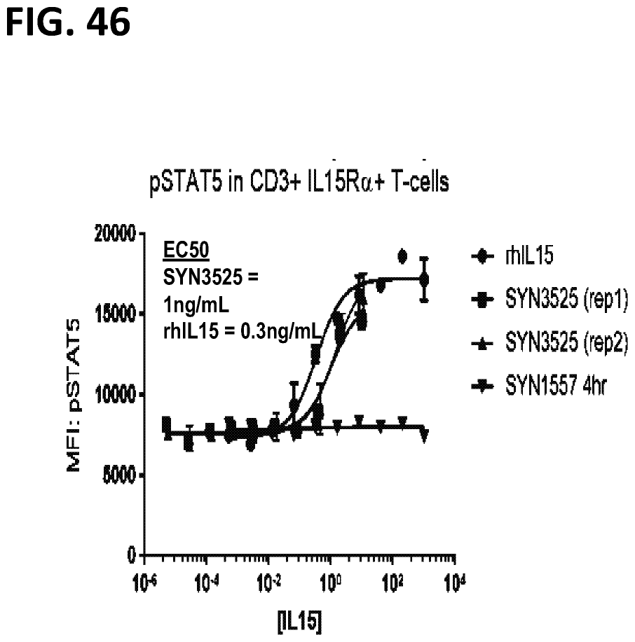

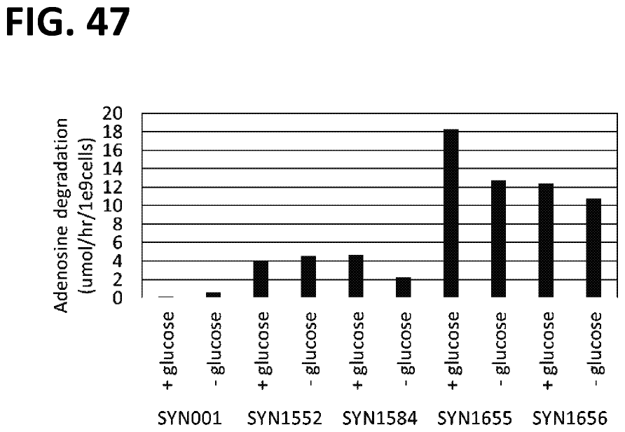

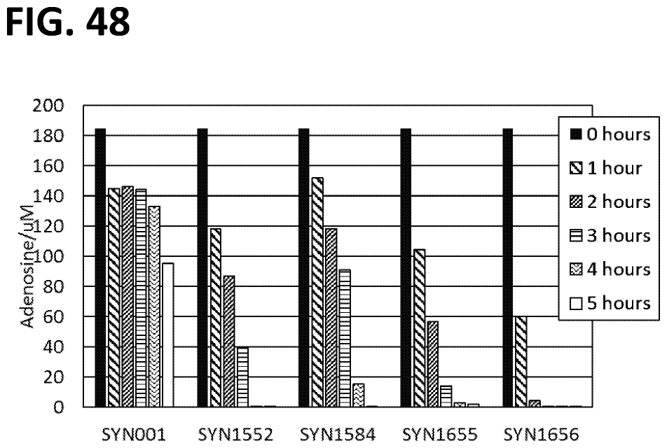

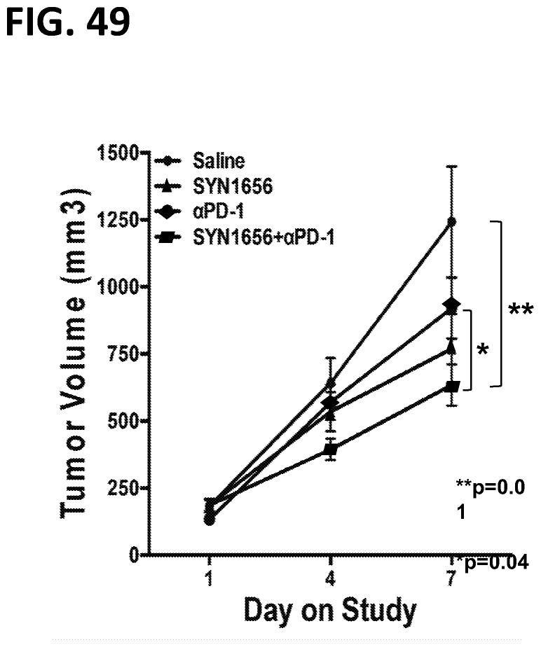

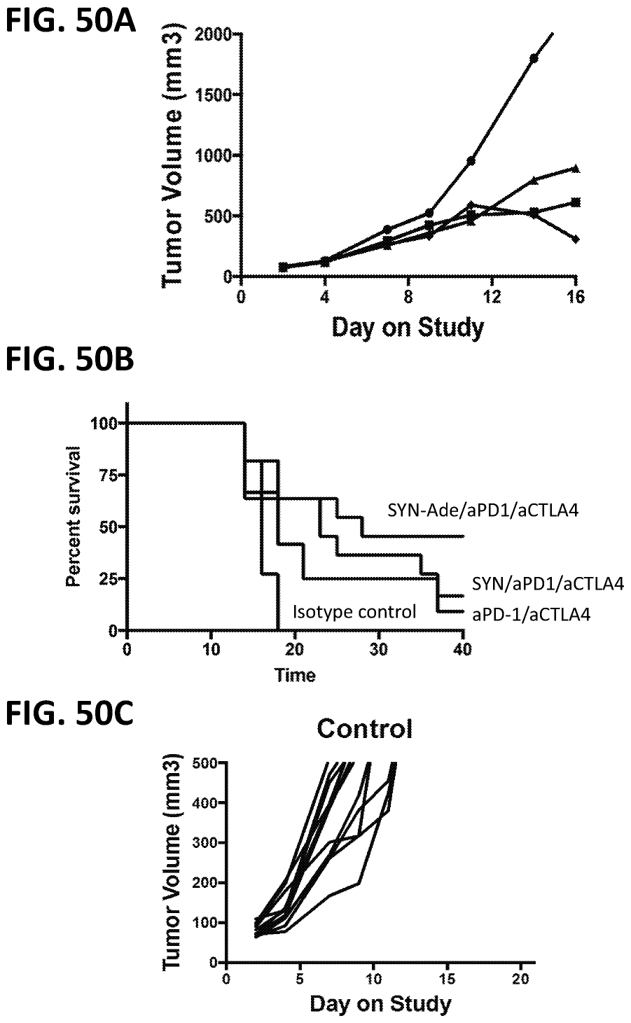

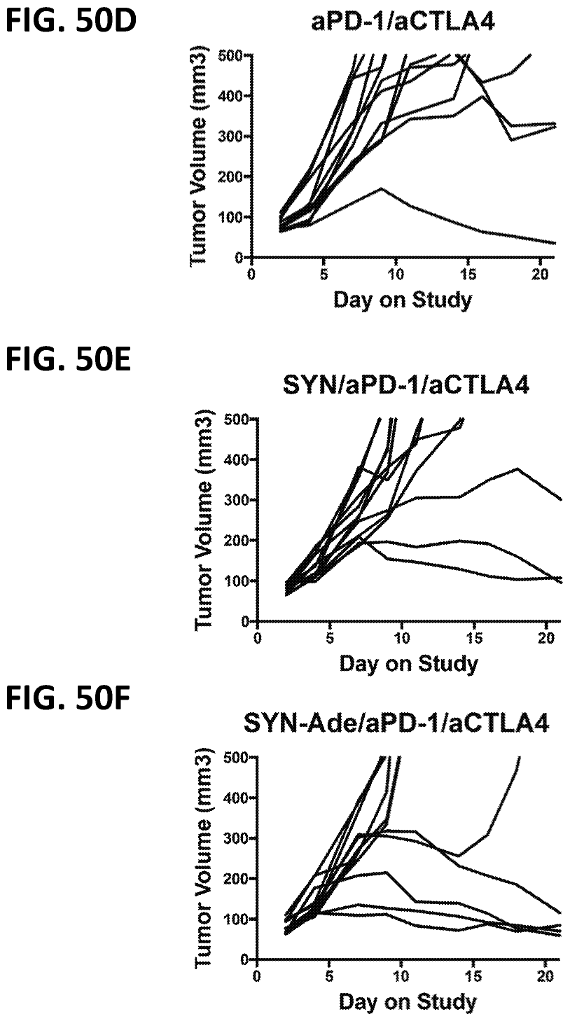

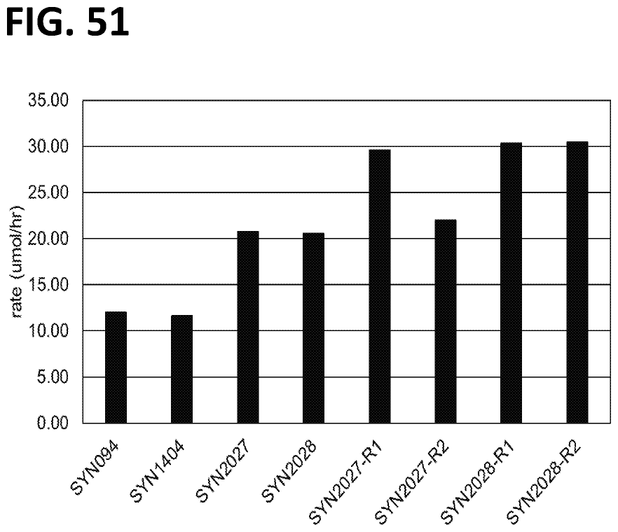

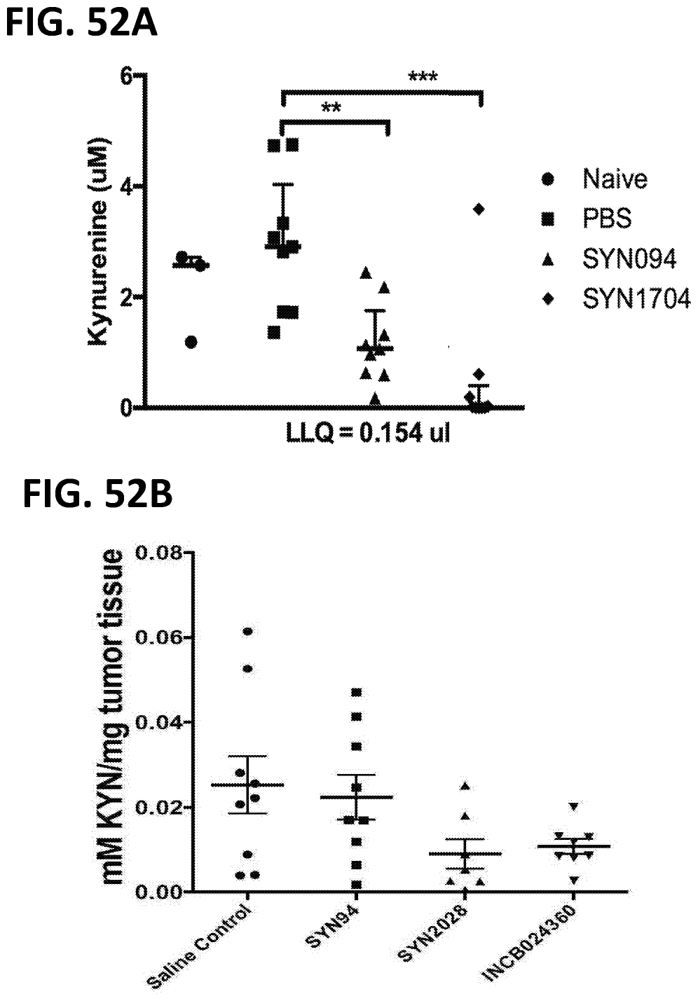

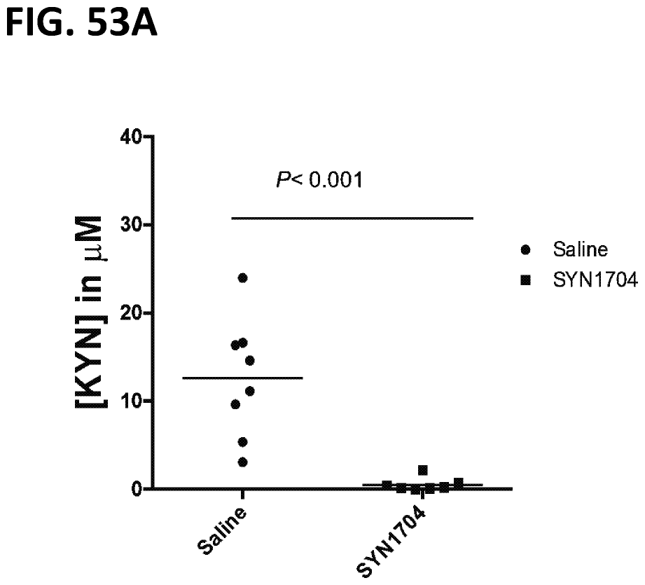

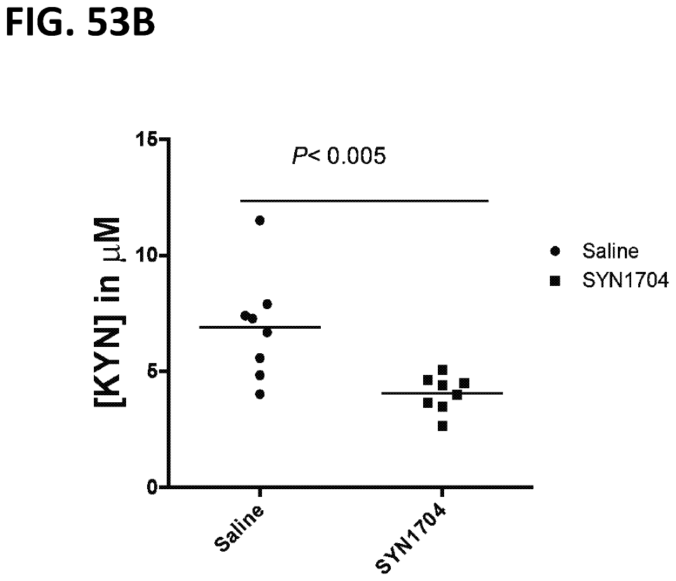

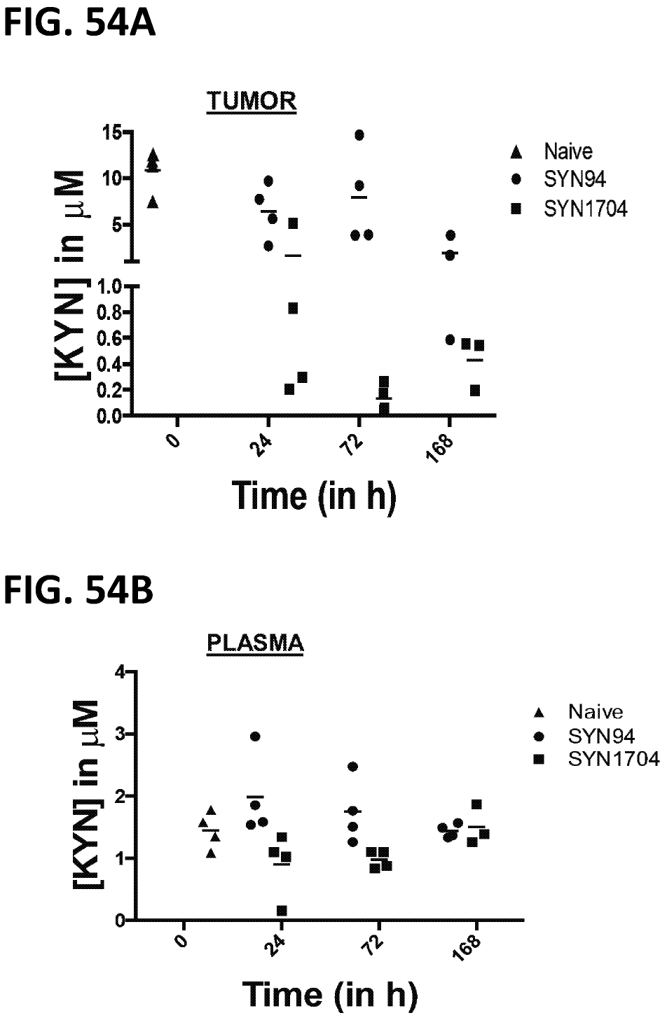

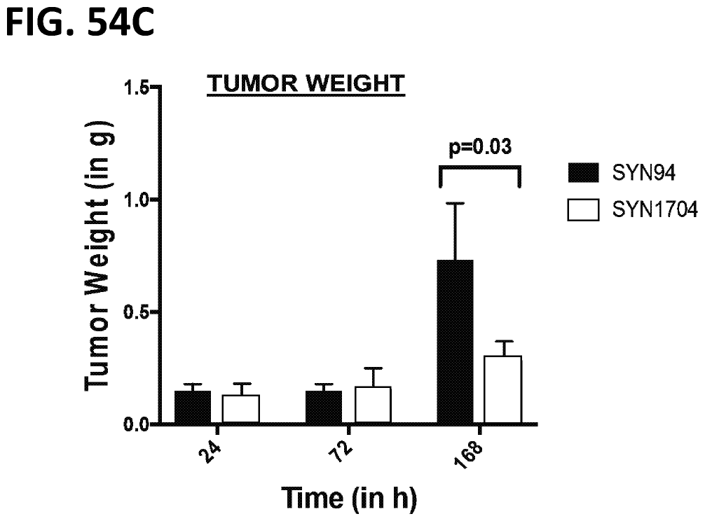

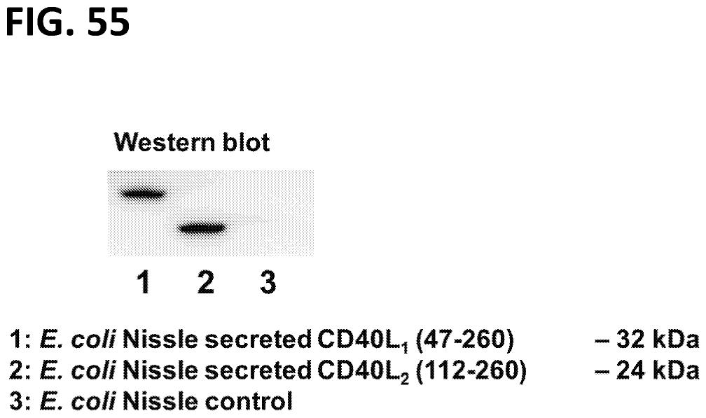

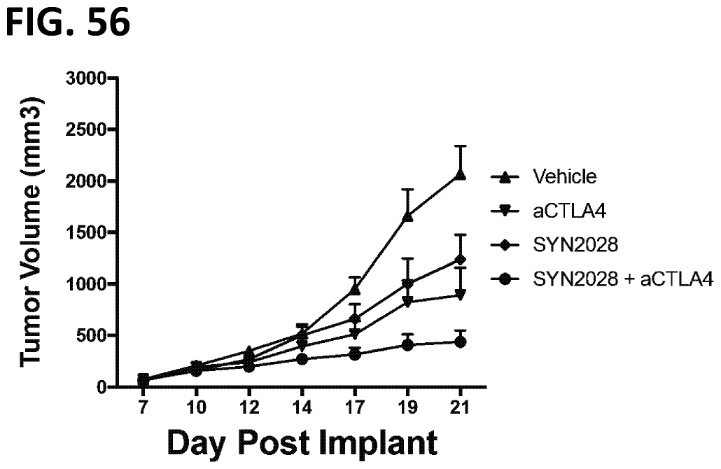

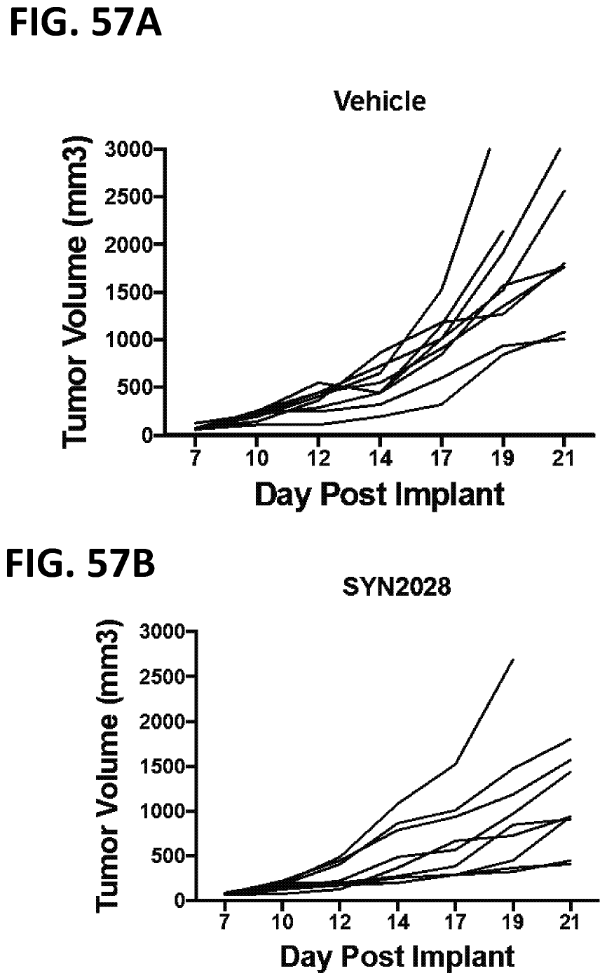

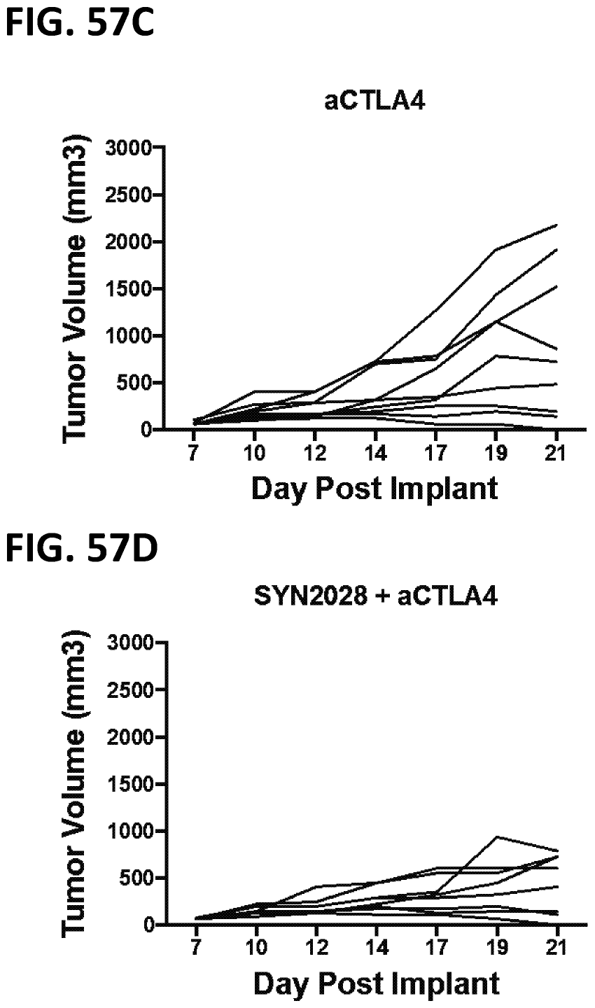

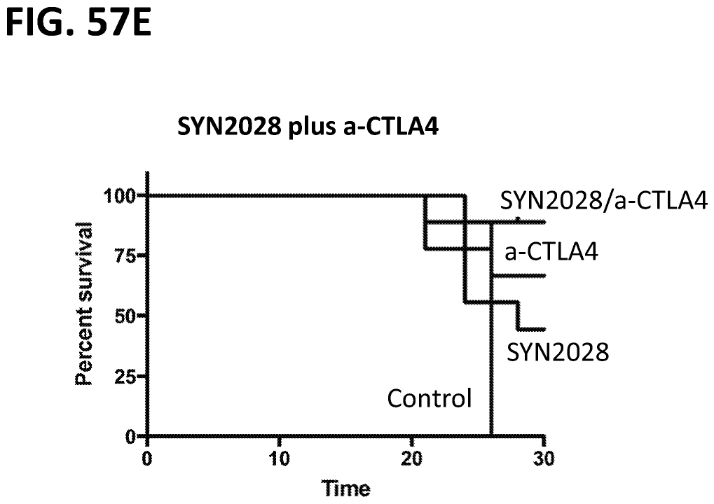

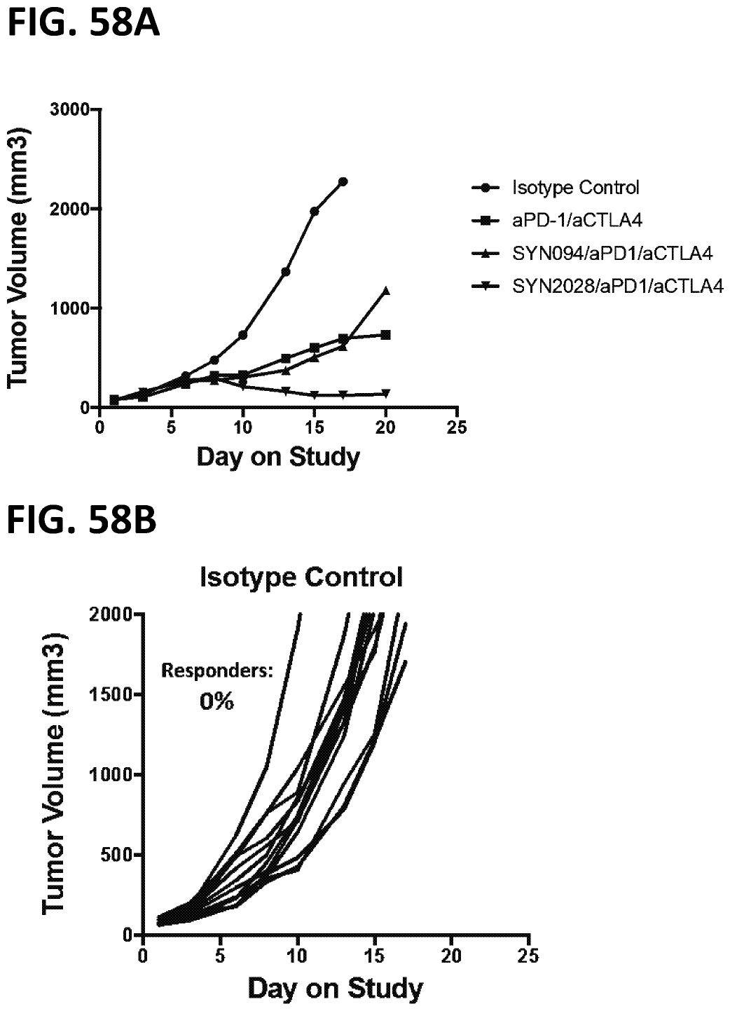

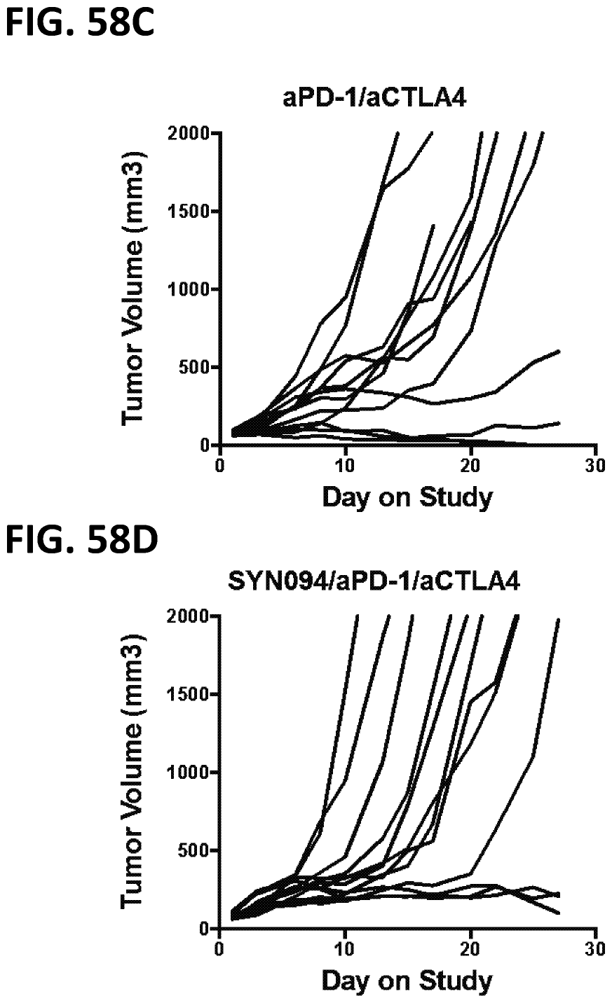

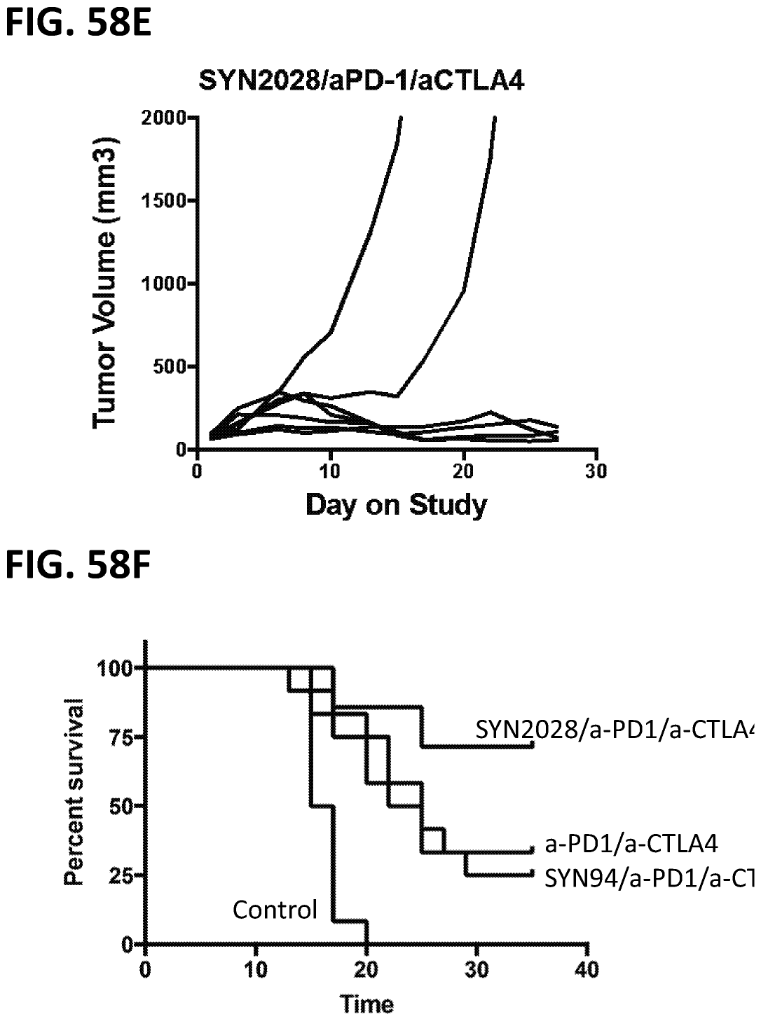

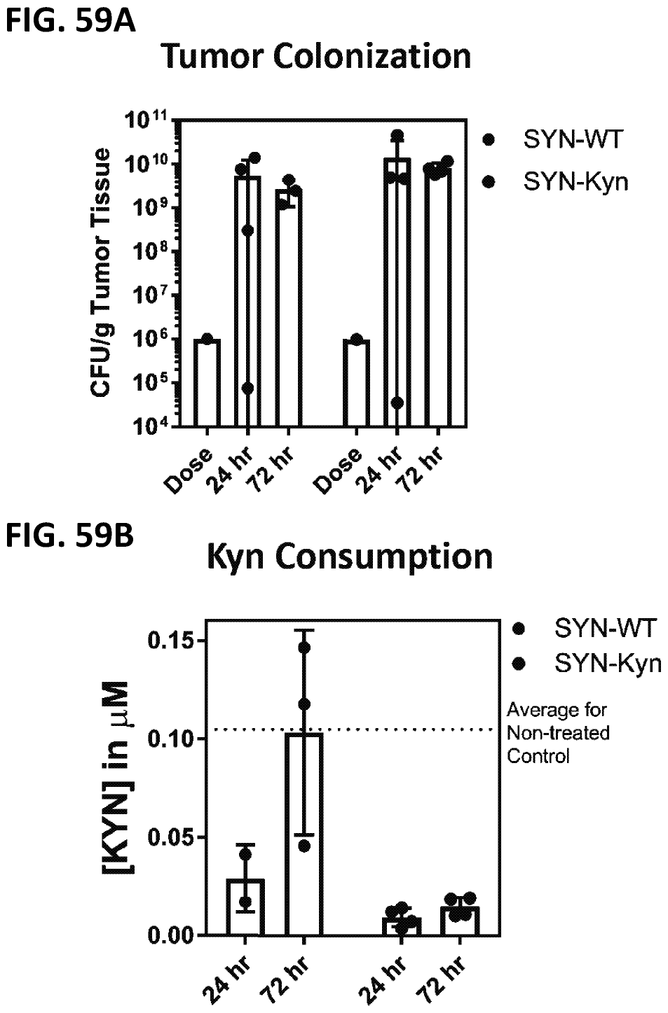

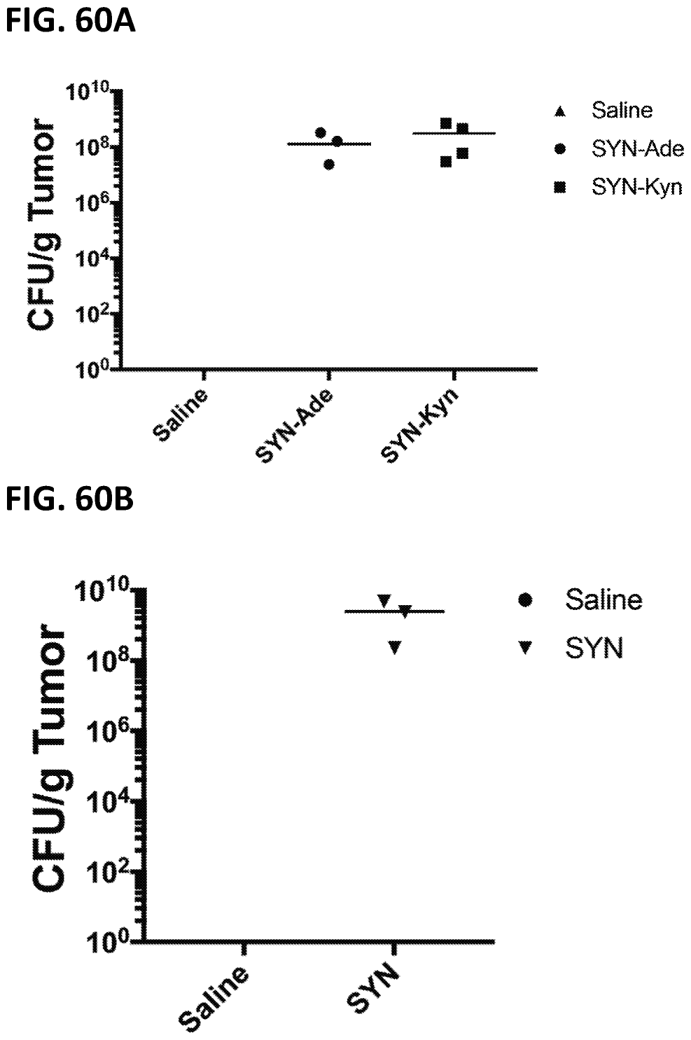

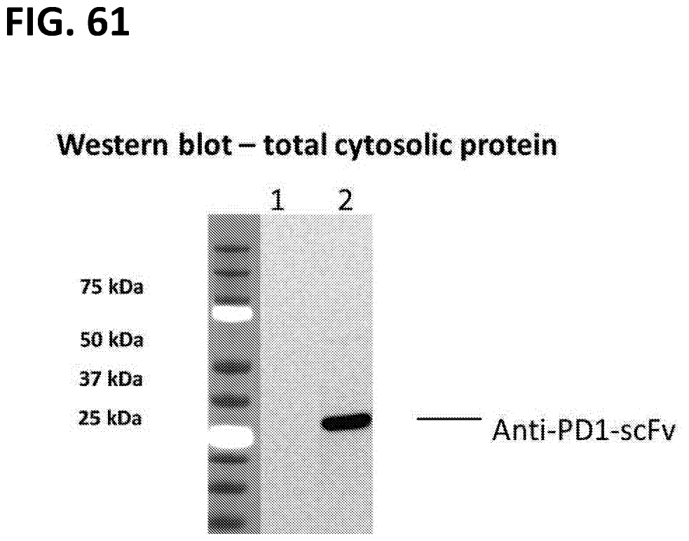

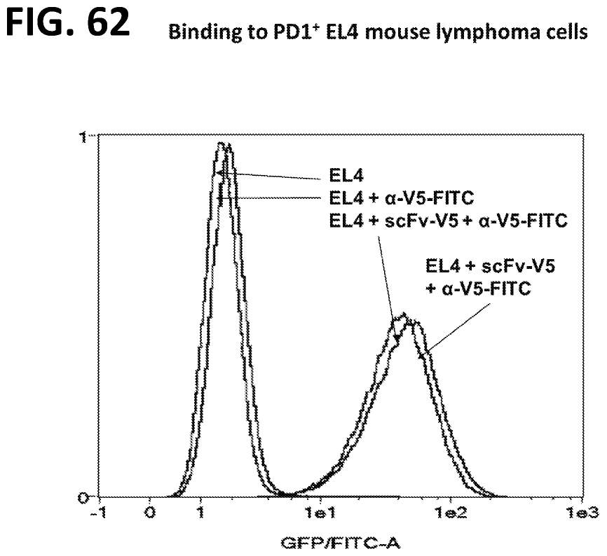

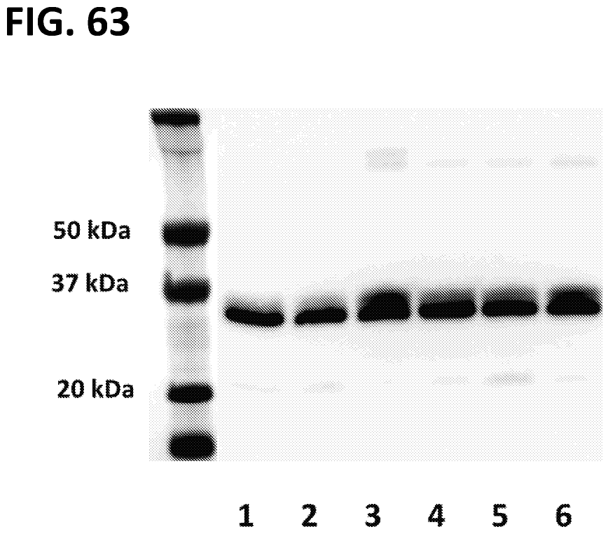

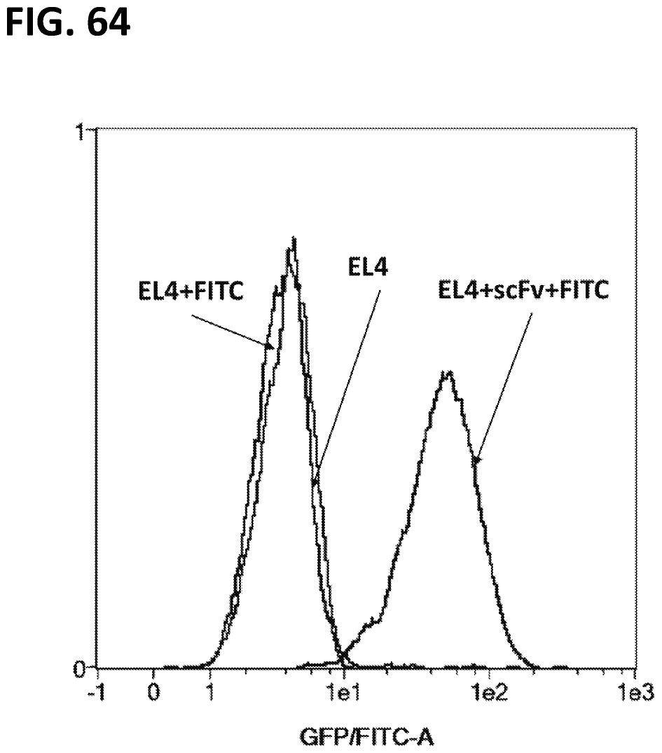

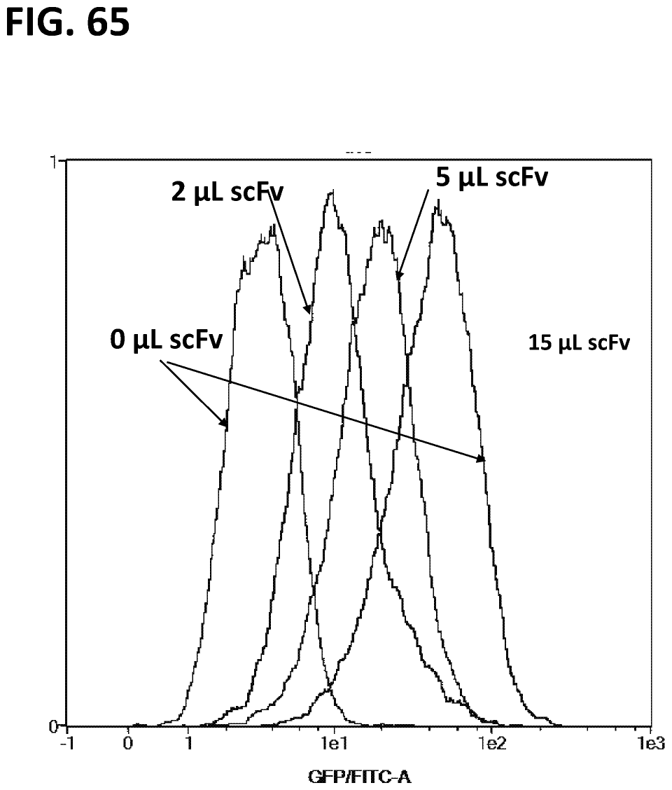

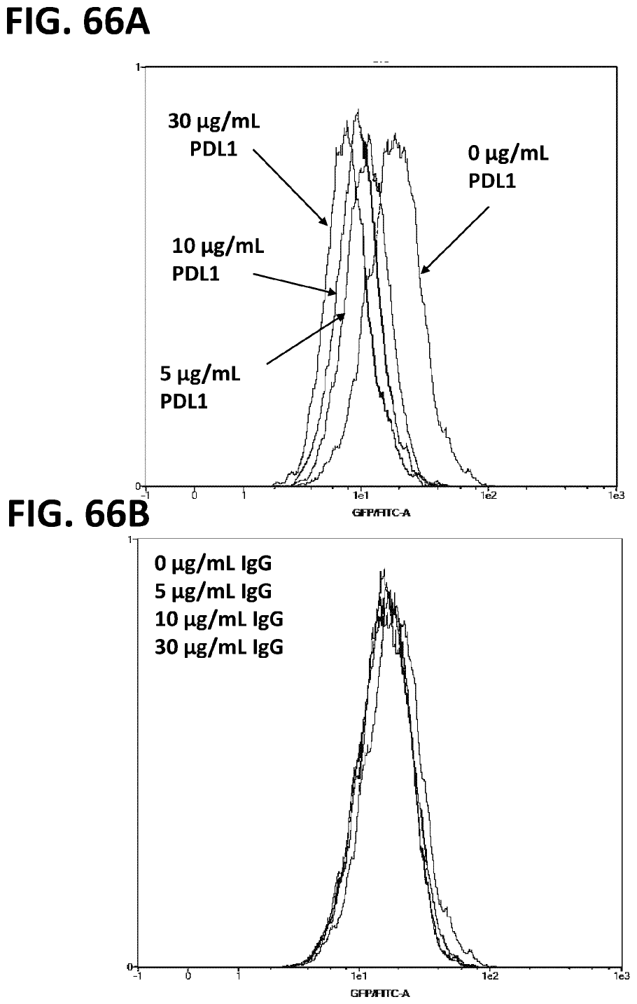

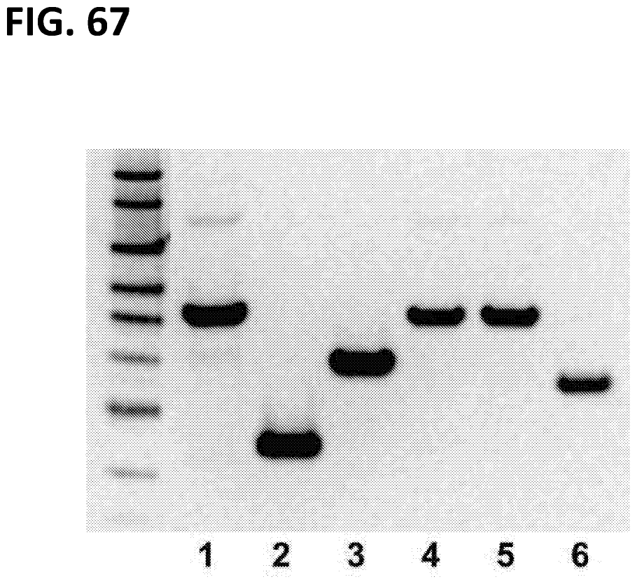

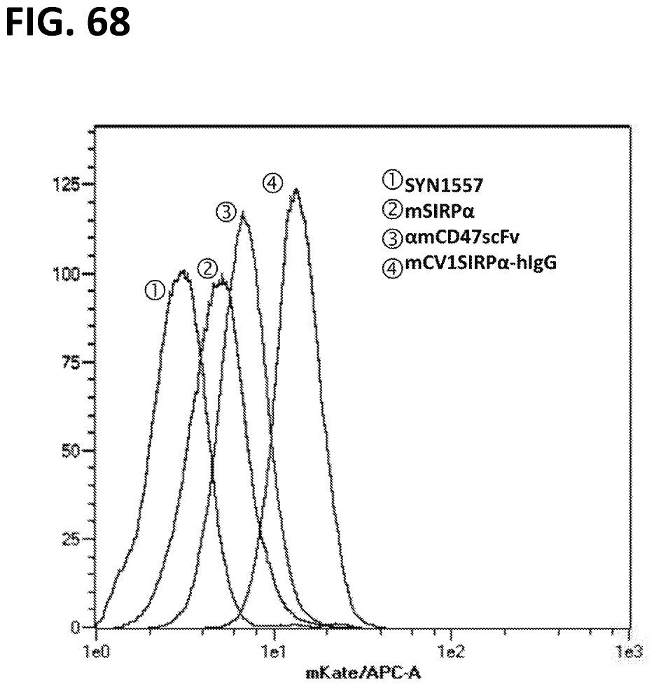

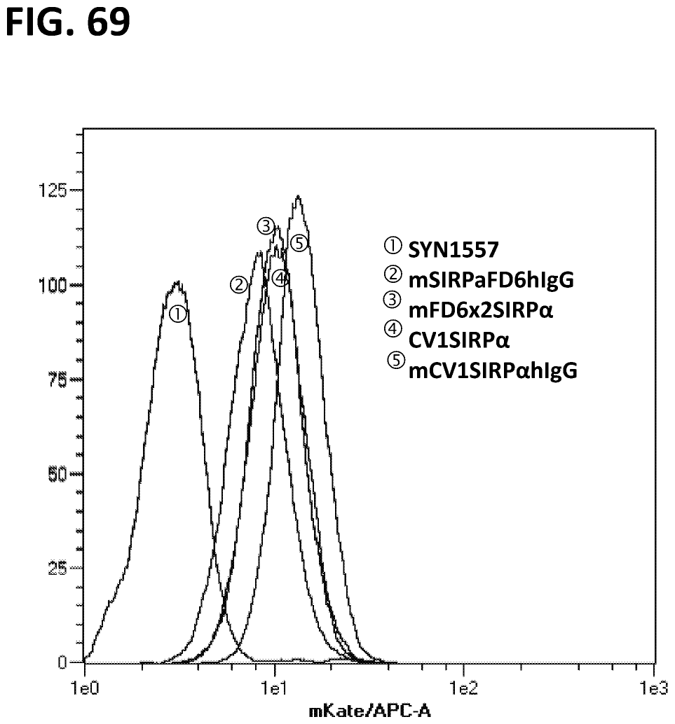

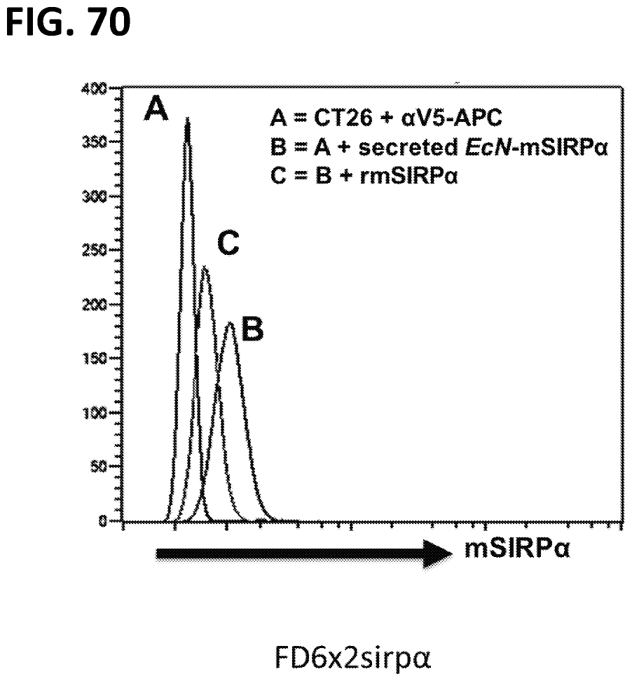

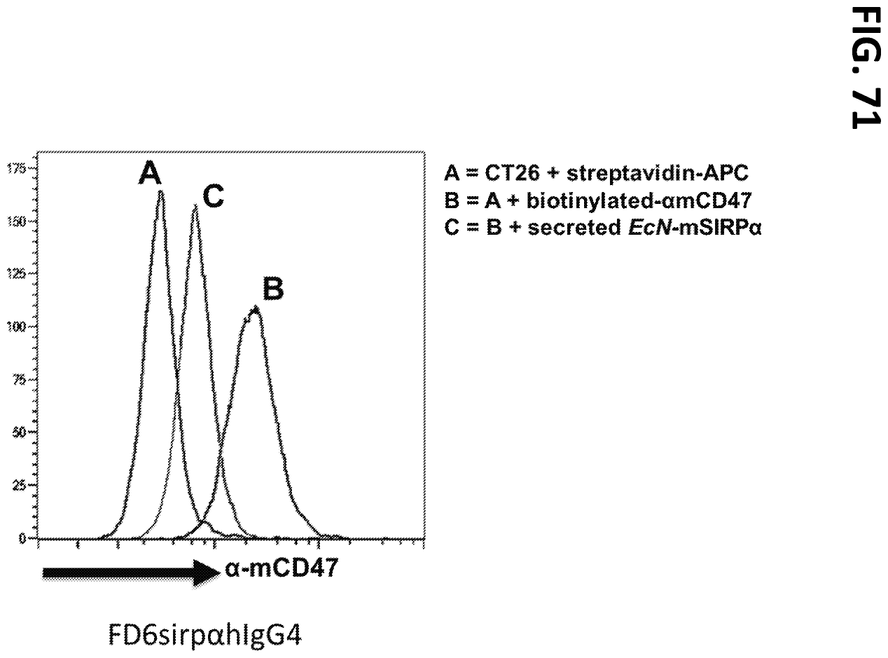

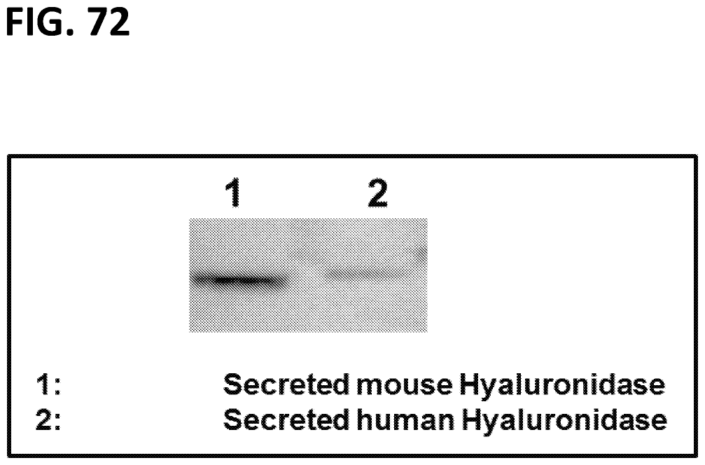

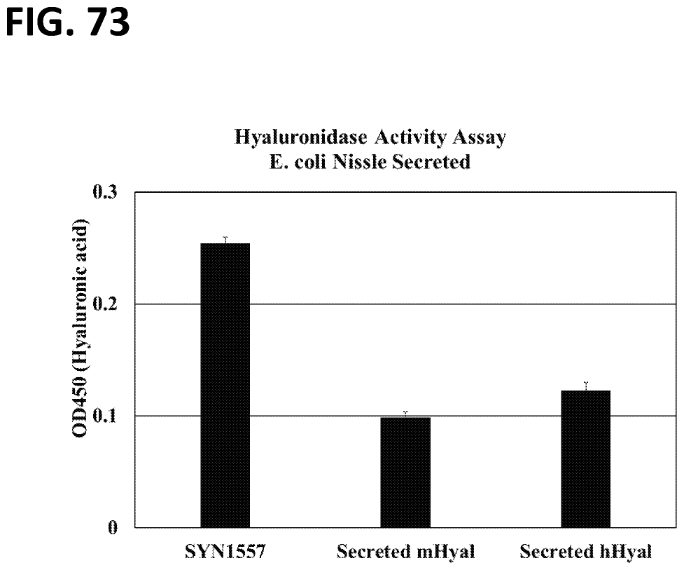

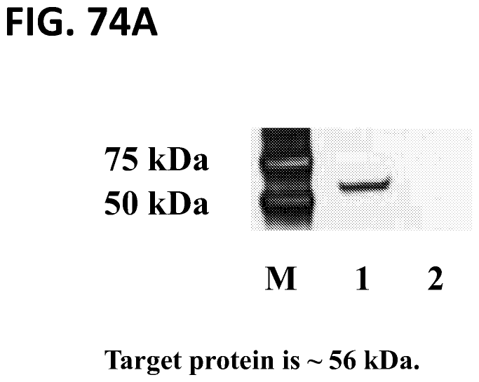

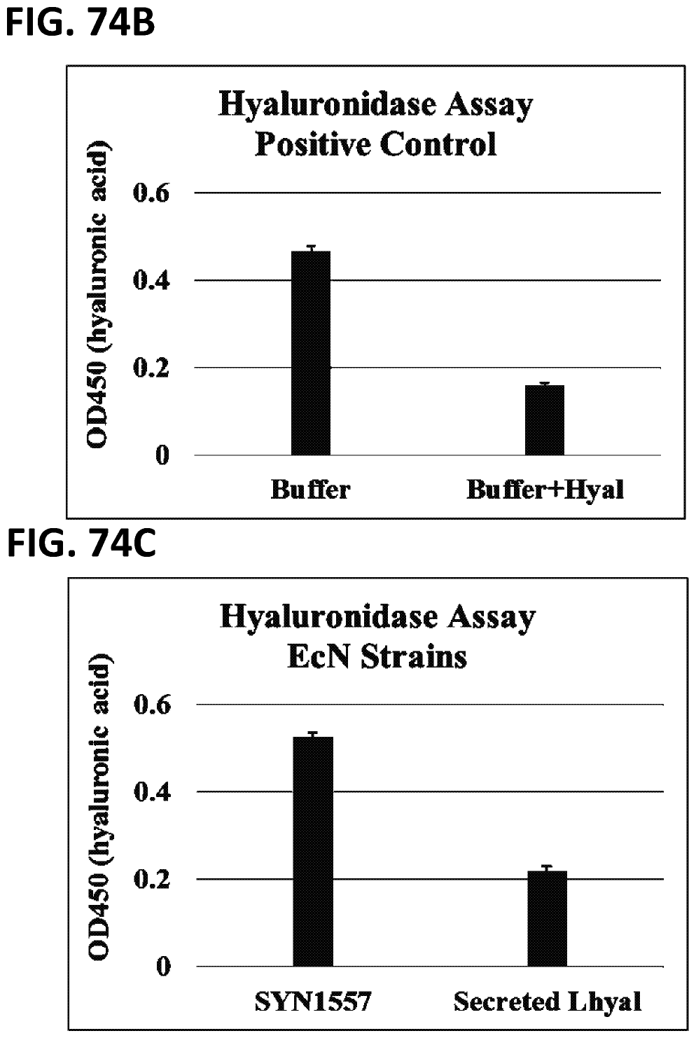

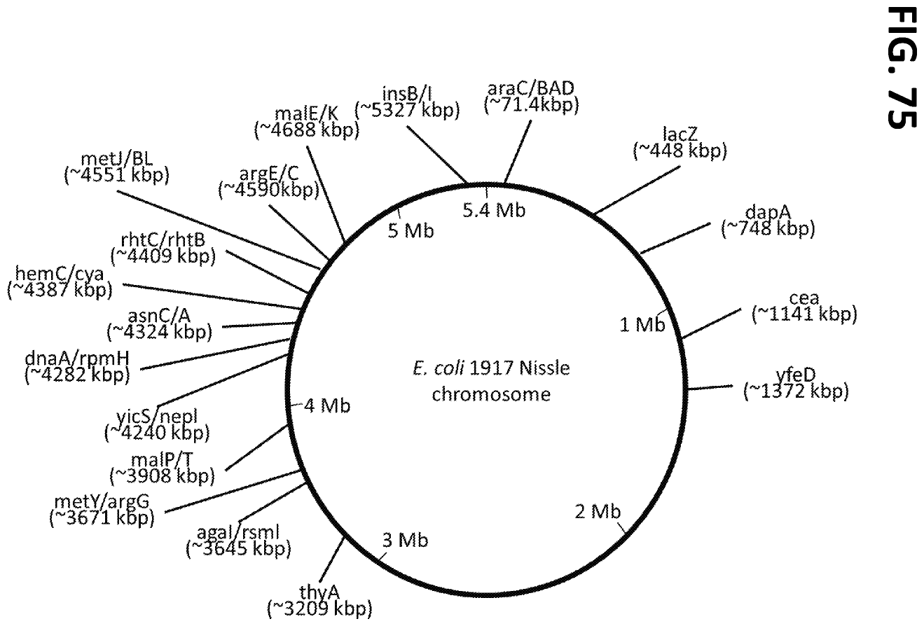

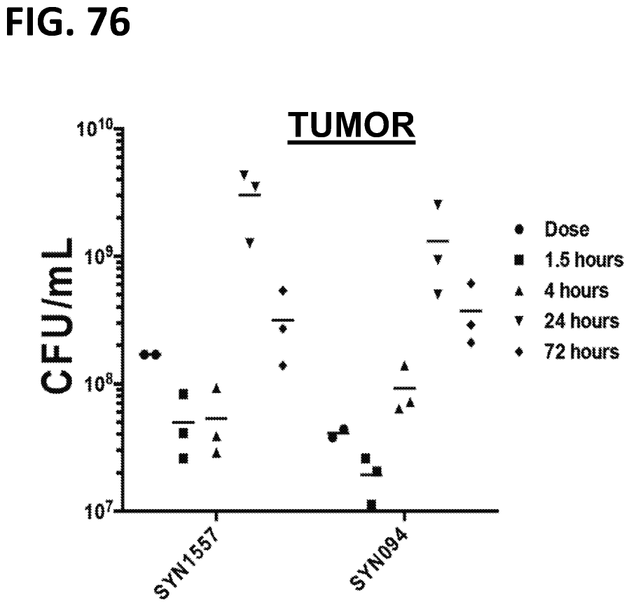

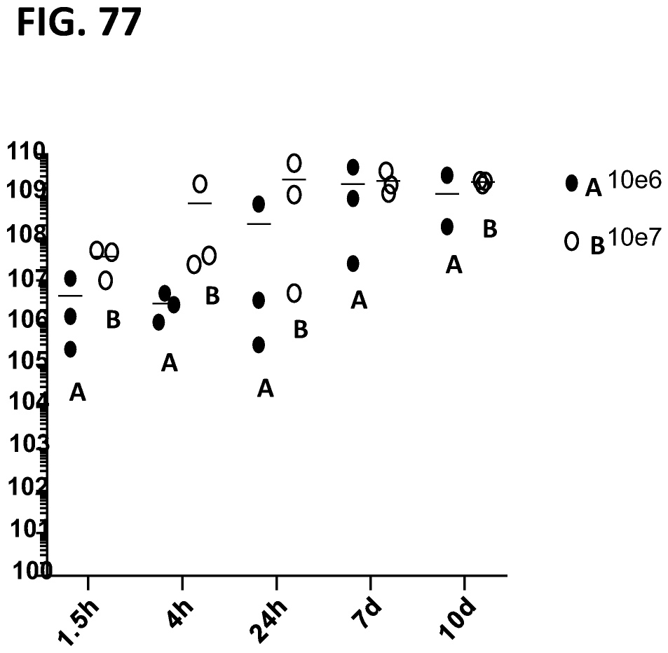

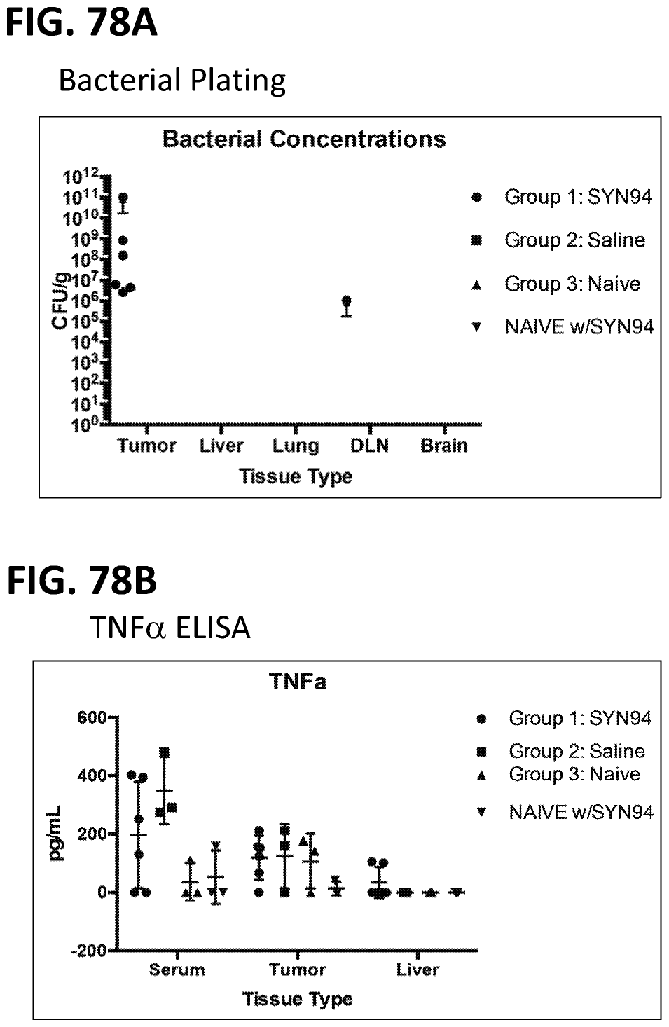

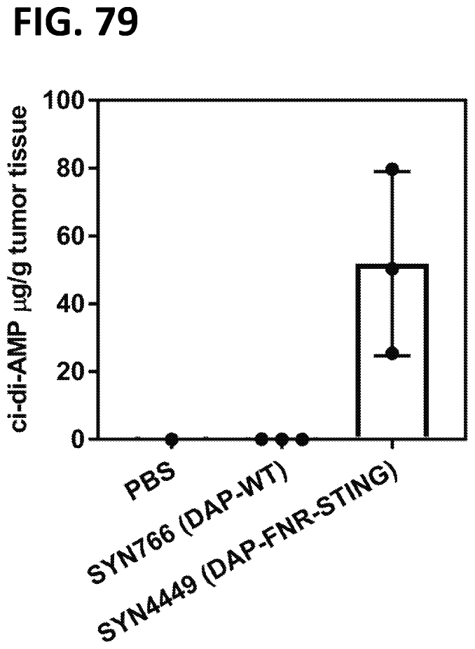

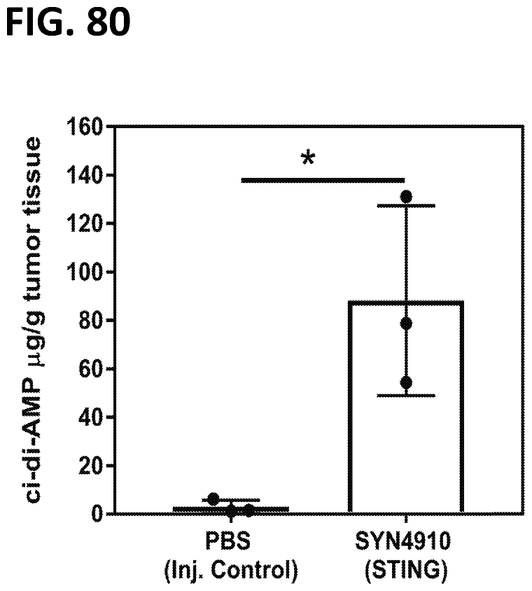

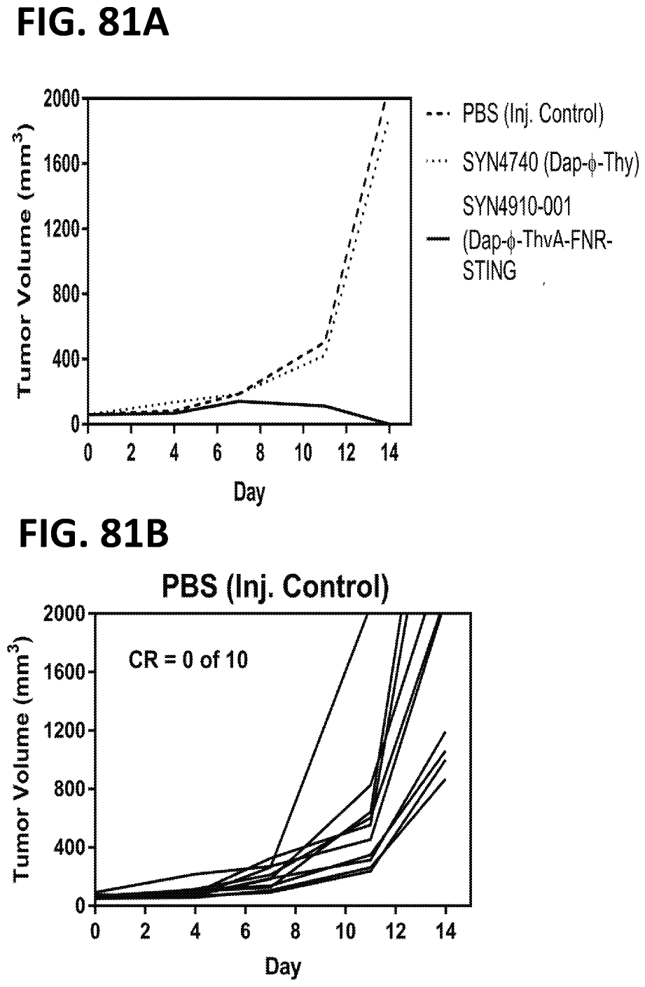

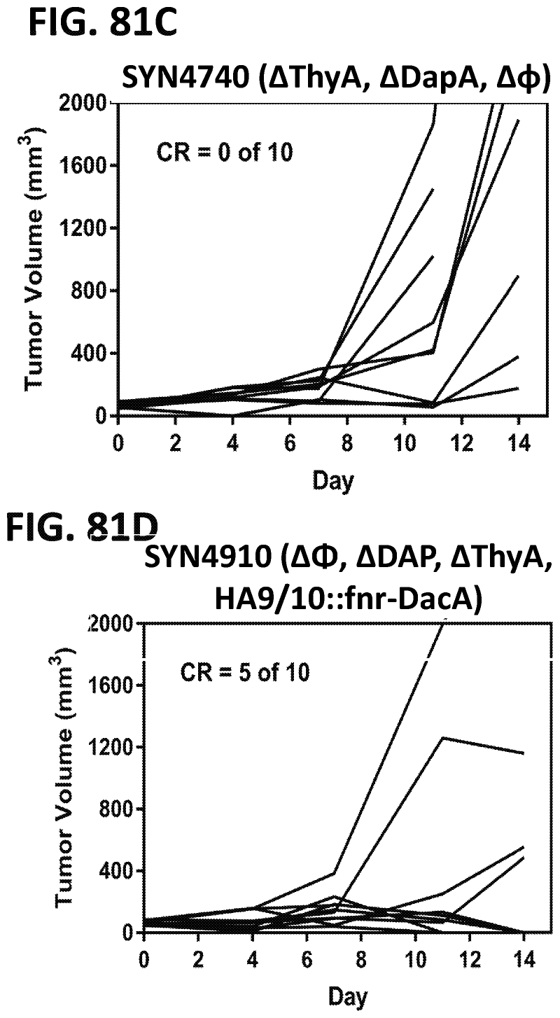

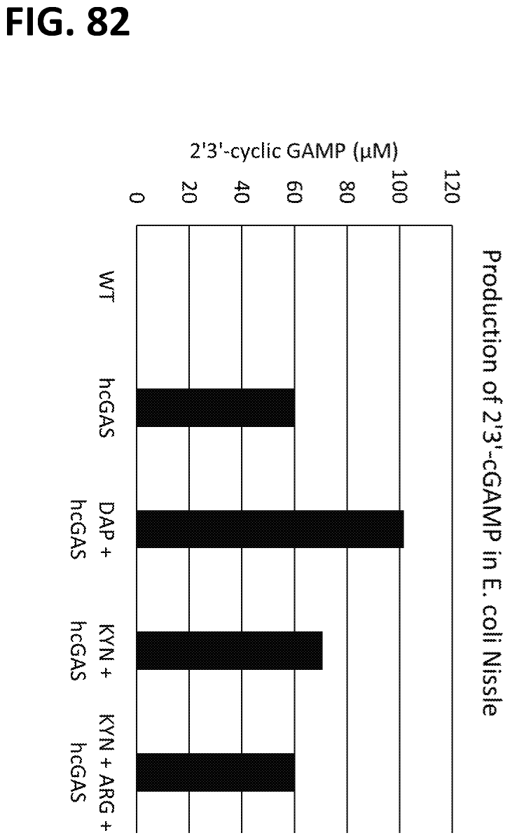

Tumor volume and body weight were measured 3 times per week and animal health was assessed three times per week. Results are depicted in FIG. 13 and show that administration of WT STING strain results in long-term immunological memory. In contrast to the naïve controls, no recurring tumors were observed in the animals previously treated with SYN3527, indicating a lasting memory response.

### Example 58. Promoter Studies (Comparison of FNR, Cumate Inducible and Salicylate Inducible Promoters

FNR, cumate-inducible and salicylate-inducible promoters were assessed for their ability to allow regulatable and efficacious effector expression levels in vitro.

The salicylate sensor circuit PSal/NahR biosensor circuit employed (Part:BBa_J61051) was originally adapted from Pseudomonas putida. The nahR gene was mined from the 83 kb naphthalene degradation plasmid NAH7 of Pseudomonas putida, encoding a 34 kDa protein which binds to nah and sal promoters to activate transcription in response to the inducer salicylate (Dunn, N. W., and I. C. Gunsalus. (1973) Transmissible plasmid encoding early enzymes of naphthalene oxidation in Pseudomonas putida. J. Bacteriol. 114:974-979). NahR is constitutively expressed by a constitutive promoter (Pc), and the expression of the protein of interest is positively regulated by NahR in the presence of inducers. Here, the Biobrick BBa_J61051 (containing the gene encoding NahR driven by a constitutive promoter and the PSal promoter was cloned preceding dacA in the backbone p15.

The basic mechanism by which the cumate-regulated expression functions in the native P. putida Fl and how it is applied to E. coli has been previously described (see e.g., Choi et al., Novel, Versatile, and Tightly Regulated Expression System for Escherichia coli Strains; Appl. Environ. Microbiol. August 2010 vol. 76 no. 15 5058-5066). Essentially, the cumate circuit or switch includes four components: a strong promoter, a repressor-binding DNA sequence or operator, expression of cymR, a repressor, and cumate as the inducer. The addition of the inducer changes causes the formation of a complex between cumate and CymR and results in the removal of the repressor from its DNA binding site, allowing expression of the gene of interest. Here a construct comprising the cymR gene driven by a constitutive promoter and a cymR responsive promoter was cloned in front of the DacA gene in p15 to allow cumate inducible expression.

The ability of these promoters to drive expression of DacA and allow production of c-di-AMP in vitro was assessed. Fragments containing FNR, cumate-inducible and salicylate-inducible promoters were cloned into p15 containing the dacA construct. Strains were designated as follows as shown in Table 60.

TABLE 60
Inducible DacA Strains

| | | | | DacA Construct | | Regulators |
|---|---|---|---|---|---|---|
| | | | | sequence | Promoter | and other |
| | Construct | Parental strain | | (SEQ ID NO) | (SEQ ID NO) | (SEQ ID NO) |
| 1 | tet-dacA | SYN94 | SYN3527-km | | | |
| 2 | salicylate-dacA | SYN94 | SYN4031-km | 1275 | 1273, 1274 | 1276, 1277, |
| | | | | | | 1278, 1279, |
| | | | | | | 1280 |
| 3 | cumate-dacA | SYN94 | SYN4340-km | 1265 | 1270, 1271 | 1266, 1267, |
| | | | | | | 1268, 1269 |
| 4 | fnr-dacA | SYN94 | SYN4448-km | 1284 | 1281, 1282 | |
| 5 | tet-dacA | SYN766 (ΔDapA) | SYN4023-cm-km | | | |
| 6 | salicylate-dacA | SYN766 (ΔDapA) | SYN4356-cm-km | 1275 | 1273, 1274 | 1276, 1277, |
| | | | | | | 1278, 1279, |
| | | | | | | 1280 |
| 7 | cumate-dacA | SYN766 (deltaDapA) | SYN4357-cm-km | 1265 | 1270, 1271 | 1266, 1267, |
| | | | | | | 1268, 1269 |
| 8 | fnr-dacA | SYN766 (deltaDapA) | SYN4449-cm-km | 1284 | 1281, 1282 | |

TABLE 61
Activity comparison between strains
with unmodified and ADapA chassis

| | | | Unmodified/ |
|---|---|---|---|
| Construct | unmodified | auxotroph | DeltaDap |
| tet-dacA | SYN3527-km | SYN4023-cm-km | 1.06 |
| salicylate-dacA | SYN4031-km | SYN4356-cm-km | 0.96 |
| cumate-dacA | SYN4340-km | SYN4357-cm-km | 1.05 |
| fnr-dacA | SYN4448-km | SYN4449-cm-km | 1.24 |

### Example 59. Generation of a ThyA Auxotrophy (ΔthyA)

An auxotrophic mutation causes bacteria to die in the absence of an exogenously added nutrient essential for survival or growth because they lack the gene(s) necessary to produce that essential nutrient. In order to generate genetically engineered bacteria with an auxotrophic modification in the genetically engineered strains, the thyA, a gene essential for oligonucleotide synthesis, is deleted. Deletion of the thyA gene in E. coli Nissle yields a strain that cannot form a colony on LB plates unless they are supplemented with thymidine. A thyA::cam PCR fragment is amplified using 3 rounds of PCR as follows. For the first PCR round, 4×50 ul PCR reactions containing 1 ng pKD3 as template, 25 ul 2×phusion, 0.2 ul primer SR36 and SR38, and either 0, 0.2, 0.4 or 0.6 ul DMSO are brought up to 50 ul volume with nuclease free water and amplified under the following cycle conditions:

step1: 98c for 30s

step2: 98c for 10s

step3: 55c for 15s

step4: 72c for 20s

repeat step 2-4 for 30 cycles

step5: 72c for 5 min

Subsequently, 5 ul of each PCR reaction is run on an agarose gel to confirm PCR product of the appropriate size. The PCR product is purified from the remaining PCR reaction using a Zymoclean gel DNA recovery kit according to the manufacturer's instructions and eluted in 30 ul nuclease free water.

For the second round of PCR, 1 ul purified PCR product from round 1 is used as template, in 4×50 ul PCR reactions as described above except with 0.2 ul of primers SR33 and SR34. Cycle conditions are the same as noted above for the first PCR reaction. The PCR product run on an agarose gel to verify amplification, purified, and eluted in 30 ul as described above.

For the third round of PCR, 1 ul of purified PCR product from round 2 is used as template in 4×50 ul PCR reactions as described except with primer SR43 and SR44. Cycle conditions are the same as described for rounds 1 and 2. Amplification is verified, the PCR product purified, and eluted as described above. The concentration and purity is measured using a spectrophotometer. The resulting linear DNA fragment, which contains 92 bp homologous to upstream of thyA, the chloramphenicol cassette flanked by frt sites, and 98 bp homologous to downstream of the thyA gene, is transformed into a E. coli Nissle 1917 strain containing pKD46 grown for recombineering. Following electroporation, 1 ml SOC medium containing 3 mM thymidine is added, and cells are allowed to recover at 37 C for 2h with shaking. Cells are then pelleted at 10,000×g for 1 minute, the supernatant is discarded, and the cell pellet is resuspended in 100 ul LB containing 3 mM thymidine and spread on LB agar plates containing 3 mM thy and 20 ug/ml chloramphenicol. Cells are incubated at 37 C overnight. Colonies that appeared on LB plates are restreaked. + cam 20 ug/ml + or − thy 3 mM. (thyA auxotrophs will only grow in media supplemented with thy 3 mM).

Next, the antibiotic resistance is removed with pCP20 transformation. pCP20 has the yeast Flp recombinase gene, FLP, chloramphenicol and ampicillin resistant genes, and temperature sensitive replication. Bacteria are grown in LB media containing the selecting antibiotic at 37° C. until OD600=0.4-0.6. 1 mL of cells are ished as follows: cells are pelleted at 16,000×g for 1 minute. The supernatant is discarded and the pellet is resuspended in 1 mL ice-cold 10% glycerol. This step is repeated 3× times. The final pellet is resuspended in 70 ul ice-cold 10% glycerol. Next, cells are electroporated with 1 ng pCP20 plasmid DNA, and 1 mL SOC supplemented with 3 mM thymidine is immediately added to the cuvette. Cells are resuspended and transferred to a culture tube and grown at 30° C. for 1 hours. Cells are then pelleted at 10,000×g for 1 minute, the supernatant is discarded, and the cell pellet is resuspended in 100 ul LB containing 3 mM thymidine and spread on LB agar plates containing 3 mM thy and 100 ug/ml carbenicillin and grown at 30° C. for 16-24 hours. Next, transformants are colony purified non-selectively (no antibiotics) at 42° C.

To test the colony-purified transformants, a colony is picked from the 42° C. plate with a pipette tip and resuspended in 10 μL LB. 3 μL of the cell suspension is pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies are considered cured if there is no growth in neither the Cam or Amp plate, picked, and re-streaked on an LB plate to get single colonies, and grown overnight at 37° C.

Subsequently, 5 ul of each PCR reaction is run on an agarose gel to confirm PCR product of the appropriate size. The PCR product is purified from the remaining PCR reaction using a Zymoclean gel DNA recovery kit according to the manufacturer's instructions and eluted in 30 ul nuclease free water.

For the second round of PCR, 1 ul purified PCR product from round 1 is used as template, in 4×50 ul PCR reactions as described above except with 0.2 ul of primers SR33 and SR34. Cycle conditions are the same as noted above for the first PCR reaction. The PCR product run on an agarose gel to verify amplification, purified, and eluted in 30 ul as described above.

For the third round of PCR, 1 ul of purified PCR product from round 2 is used as template in 4×50 ul PCR reactions as described except with primer SR43 and SR44. Cycle conditions are the same as described for rounds 1 and 2. Amplification is verified, the PCR product purified, and eluted as described above. The concentration and purity is measured using a spectrophotometer. The resulting linear DNA fragment, which contains 92 bp homologous to upstream of thyA, the chloramphenicol cassette flanked by frt sites, and 98 bp homologous to downstream of the thyA gene, is transformed into a E. coli Nissle 1917 strain containing pKD46 grown for recombineering. Following electroporation, 1 ml SOC medium containing 3 mM thymidine is added, and cells are allowed to recover at 37 C for 2h with shaking. Cells are then pelleted at 10,000×g for 1 minute, the supernatant is discarded, and the cell pellet is resuspended in 100 ul LB containing 3 mM thymidine and spread on LB agar plates containing 3 mM thy and 20 ug/ml chloramphenicol. Cells are incubated at 37 C overnight. Colonies that appeared on LB plates are restreaked. + cam 20 ug/ml + or − thy 3 mM. (thyA auxotrophs will only grow in media supplemented with thy 3 mM).

Next, the antibiotic resistance is removed with pCP20 transformation. pCP20 has the yeast Flp recombinase gene, FLP, chloramphenicol and ampicillin resistant genes, and temperature sensitive replication. Bacteria are grown in LB media containing the selecting antibiotic at 37° C. until OD600=0.4-0.6. 1 mL of cells are ished as follows: cells are pelleted at 16,000×g for 1 minute. The supernatant is discarded and the pellet is resuspended in 1 mL ice-cold 10% glycerol. This step is repeated 3× times. The final pellet is resuspended in 70 ul ice-cold 10% glycerol. Next, cells are electroporated with 1 ng pCP20 plasmid DNA, and 1 mL SOC supplemented with 3 mM thymidine is immediately added to the cuvette. Cells are resuspended and transferred to a culture tube and grown at 30° C. for 1 hours. Cells are then pelleted at 10,000×g for 1 minute, the supernatant is discarded, and the cell pellet is resuspended in 100 ul LB containing 3 mM thymidine and spread on LB agar plates containing 3 mM thy and 100 ug/ml carbenicillin and grown at 30° C. for 16-24 hours. Next, transformants are colony purified non-selectively (no antibiotics) at 42° C.

To test the colony-purified transformants, a colony is picked from the 42° C. plate with a pipette tip and resuspended in 10 μL LB. 3 μL of the cell suspension is pipetted onto a set of 3 plates: Cam, (37° C.; tests for the presence/absence of CamR gene in the genome of the host strain), Amp, (30° C., tests for the presence/absence of AmpR from the pCP20 plasmid) and LB only (desired cells that have lost the chloramphenicol cassette and the pCP20 plasmid), 37° C. Colonies are considered cured if there is no growth in neither the Cam or Amp plate, picked, and re-streaked on an LB plate to get single colonies, and grown overnight at 37° C.

In other embodiments, similar methods are used to create other auxotrophies, including, but not limited to, dapA.

### Example 60. Generation of a DAPA Auxotroph STING Strain

To control growth in vivo and in the environment, the parental E. coli Nissle chassis was engineered to be an auxotrophic strain through a deletion of the dapA gene, encoding 4-hydroxy-tetrahydropicolinate synthase, which is essential for bacterial growth. This deletion renders the bacterium unable to synthesize diaminopimelate (DAP), thereby preventing the proper formation of bacterial cell wall unless the strain is supplemented with DAP exogenously.

For creation of the dapA deletion (ΔdapA), two rounds of PCR were performed using nested primers. For the first round of PCR, pKD3 was used as the template DNA. The primers were designed to generate a dsDNA fragment that contained homology adjacent to the dapA gene locus in the EcN chromosome and a chloramphenicol resistance gene flanked by frt sites. The primers used in the second round of PCR utilized the PCR product of the first round as template DNA. These primers contained additional homology to dapA to provide a greater length of EcN homology for recombineering. The resulting dapA knockout fragment included 68 base pairs and 70 base pairs of EcN homology, respectively, on its 5′ and 3′ end. A strain which contains pKD46, was transformed with the dapA knockout fragment by electroporation. Colonies were selected on LB agar containing chloramphenicol (30 μg/mL) and diaminopimelate (100 μg/mL) and correct recombination events were verified by PCR. pKD46 was cured from the strain by passage at 37 C.

This DAP auxotroph chassis was used to generate bacteria expressing gene sequences for the production of effectors, such as the STING agonist producing strain SYN4023.

### Example 61. Systemic Anti-Tumor Immunity: DAP-STING Elicits Abscopal Effects in Combination with an Agonistic Anti-OX40 Antibody and Results in Long-Term Immunological Memory

Positive costimulatory receptors that promoter T-cell activation include CD28, CD137 (also known as 4-1BB), and OX40 (also known as CD134 or Tumor necrosis factor receptor (TNFR) superfamily member 4). Recently, it has been shown that innate immune stimulation (for example though the Toll-Like Receptors) in combination with an agonistic OX40 antibody are capable of promoting an T cell dependent (CD4+ and CD8+ T cell dependent) abscopal effect and immunological memory (Sagiv-Barfi et al., In Situ Vaccination with a TLR9 Agonist and Anti-OX40 Antibody Leads to Tumor Regression and Induces Abscopal Responses in Murine Lymphoma; Blood 2016 128:1847; Sagiv-Barfi et al., Eradication of spontaneous malignancy by local immunotherapy Science Translation Medicine, 2018). The ability of SYN4023 (comprising plasmid-based tetracycline inducible DacA and a dapA auxotrophic mutation) to induce an abscopal effect in combination with an anti-OX40 antibody was assessed in the A20 tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with A20 tumors (50:50 Matrigel:PBS) 2e5 cells/mouse SC into the right flank and 1e5 cells/mouse into the left flank of each animal on Day −14. On Day −3, tumor growth was monitored; when the tumors reached ˜80-100 mm³, mice (Day 0) were randomized into experimental groups (N=10 per group) for intratumor dosing as shown in Table 61.

TABLE 62
Experimental groups

| Group No | | |
|---|---|---|
| (n = 12) | Treatment | Dosing Schedule |
| 1 | Saline | 40 uL saline day 1 and 4 |
| 2 | SYN4023 (ptet-DacA; | [1e8 CFU] in 40 uL, |
| | deltaDapA; 1e8) plus | 400 ug anti-OX40 in 40 uL; |
| | anti-OX40 at 400 | 3 doses for each on d 0 3 and 7 |
| | μg per dose)]] | |

Animals were dosed with SYN4023 bacteria I.T. and OX40 antibody I.T. for 3 doses on d0, 3 and 7. Mice were dosed with 10 ug ATC (anhydrotetracycline) via intraperitoneal injection at 4 hours post dose.

Bacterial dosing was stopped for complete responders. Tumor volume was measured in both flanks, and animal health was assessed 3x/week. Results are shown in FIG. 23A-FIG. 23C and indicate that treatment of A20 tumors with SYN4023 elicits abscopal effects in combination with aOX40. No significant effect on body weight was observed (FIG. 23E). The survival, i.e., the length of time animals could remain on study without having to be removed due to tumor burden is shown in FIG. 23D.

Next, to test whether the complete regressions elicited by SYN4023 in the above study result in long lasting immunological memory, a re-challenge study was conducted with A20 and CT26 tumors. Animals with complete regression from SYN4023 treatment were used and compared to naïve age-matched mice.

Briefly, four 14 week old naïve Balb/c females were inoculated on the left flank with 2×105 A20 cells in 50% Matrigel and on the right flank with 1×105 CT26 cells in 50% matrigel. Mice with tumor remission (n=11) from the first part of the study described above were inoculated in a similar fashion. Mice were monitored 2-3 times per week for tumor volume, body weight and health observations.

Results are depicted in FIG. 23F-H, and FIG. 23I and show that administration of SYN4023 strain results in long-term immunological memory which completely protects against the A20 re-challenge. FIG. 23F depicts the average median tumor volume for each treatment group, and FIG. 23G and FIG. 23H depict line graphs showing the tumor volumes of the individual mice over time. FIG. 23I depicts the entirety of the 2-part study querying the abcopal effect and immunological memory potential (rechallenge with A20 is depicted). In the A20 re-challenge study arm, no recurring tumors were observed in the animals previously treated with SYN4023, in contrast to the naïve controls, indicating a lasting memory response. In the CT26 re-challenge study arm, administration of SYN4023 strain partially protected from CT26 tumor growth. Out of 11 animals previously treated with SYN4023 and challenged with CT26, 9 were protected from tumor re-growth.

### Example 62. Dose Dependent Anti-Tumor Activity of SYN4449 (DAP-FNR STING) in B16.F10 Tumor Model

The Dap auxotrophic strain, constructed as described elsewhere herein, comprising a plasmid based construct expressing dacA under the control of the FNR promoter (SYN4449; ΔDAP, p15A-fnr-dacA) was assessed at various doses in the B16.F10 melanoma tumor model.

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Mice were implanted with B16 tumors, and mice injected intratumorally with bacteria producing STING agonists at various doses and controls.

Briefly, B16 cells were implanted (2×10e5 cells/mouse in PBS) SC into the right flank of each animal on day −9. On day Day −3 tumor growth was monitored; when the tumors reached ˜50-80 mm\hat{ }3 on day 1, mice were randomized into groups (N=9 per group) for intratumor dosing as follows: Group 1—PBS (control); Group 2—SYN4449, 1e7 CFU; Group 3—SYN4449, 1e8 CFU; and Group 4-SYN4449, 1e9 CFU Animals were dosed with appropriate bacteria based on group or PBS (to control for injection) on days 1 and 4. Bacterial dosing was stopped for complete responders.

Tumor volumes at day 4, 7, 10, 12 and 15 are shown in FIGS. 26A-26D for the individual mice. Results indicate that administration of SYN4449 has a dose dependent impact on B16-F10 tumor growth and tumor rejection. At a dose of 1e9 (FIG. 26D), this treatment results in the largest percentage of rejections and significant control of tumor growth over this time period.

### Example 63. SYN4449 Dose Response in A20 Tumor Model

To determine in vivo activity and efficacy of SYN4449 (comprising FNR-dacA, delta dapA (ΔDAP, p15A-fnr-dacA)), over time at three different doses and compared to PBS control in the A20 tumor model (B-cell lymphoma model).

To produce cells for the study, overnight cultures were used to inoculate 500 mL LB medium with antibiotic. The strains were incubated with shaking at 37 C until the culture reached the end of log phase (OD600=0.8-1.0). To harvest, cells were spun down at 5000 rpm for 20 min, media was aspirated, cells were washed with PBS, resuspended in 15% Glycerol and PBS, aliquoted and frozen at −80 C. Cells were concentration tested by serial plating.

Female Balb/c mice 6 weeks of age were implanted with A20 tumors, injected intratumorally with three different doses of bacteria producing enzymes capable of producing c-diAMP. Tumor volumes were measured at various time points.

Briefly, A20 cells were implanted (2×10 5/mouse/100 μL, in PBS) SC into the right flank of each animal on day −15. Tumor growth was monitored until the tumors reached ˜60-80 mm\hat{ }3. On day 0, mice were randomized into groups (N=10 per group) for intratumor dosing as follows: PBS (group 1, vehicle control), SYN4449 (group 2, 1e6 CFU), SYN4449 (group 3, 1e7 CFU), and SYN4449 (group 4, 1e8 CFU). Animals were dosed with appropriate bacteria based on group or PBS (to control for injection) on day 0, 3 and 7. Tumor volumes and body weights were recorded three times in a week with a gap of 1-2 days in between two measurements for 30 days. Results are shown in FIGS. 27A-27D indicate that administration of SYN4449 drives dose-dependent tumor control in A20 lymphoma model, with SYN4449 at 1e6 (FIG. 27A), 1e7 (FIG. 27B), and 1e8 CFU (FIG. 27C) resulting in 5 out of 10 complete responses, 6 out of 10 complete responses and 6 out of 10 complete responses, respectively.

### Example 64. Construction and Activity of a STING Agonist Producing Strains Under Control of a Low Oxygen Promoter

To generate a STING agonist producing strain which is induced under low oxygen conditions, e.g., as exist in the tumor microenvironment, dacA from Listeria monocytogenes was cloned into a low copy plasmid under control of an FNR inducible promoter and transformed into a Nissle strain which further comprises a deletion in DapA to generate SYN4449 (ΔDAP, 15A-fnr-dacA).

To measure in vitro activity of SYN4449, overnight cultures of SYN4449 and wild type control were grown in 2YT (supplemented with diaminopimelic acid) at 37 C, shaking at 250 rpm. Cultures were back diluted 1:100 (10 mL in 125 mL baffled flask) and grown for 2-3 hours to early log phase. Once cultures reached early log, cultures were moved into a Coy anaerobic chamber supplying an anaerobic atmosphere (85% N2, 10% CO2, 5% H2). Cultures were incubated anaerobically for 3-4 hours or shaked at 37 C, 250 rpm overnight in tubes to allow for induction of dacA. Next, samples were centrifuged for 5 minutes at 10000×g to separate cellular and extracellular fractions. Cell pellets were then used to determine intracellular cyclic-di-AMP. Concentrations were determined via LC/MS as described herein. Results are shown in FIG. 28A, and show that SYN4449 can produce c-di-AMP in vitro under control of the FNR promoter.

### Example 65. Construction of Various STING Agonist Producing Strains Under Control of a Low Oxygen Promoter

Next, strains which comprise an integrated copy of FNR-DacA and/or human cGAS were constructed. Examples are listed in Table 63. Strains were constructed with dapA and/or ThyA auxotrophies, strains with both dacA and kynurenine consumption circuits were designed, and in some instances, E. coli Nissle prophage 3 was disrupted using methods described herein and known in the art.

TABLE 63
Experimental groups

| | | |
|---|---|---|
| 1 | SYN4910 | ΔΦ, ΔDAP, ΔThyA, HA9/10::fnr-DacA |
| 2 | SYN4739 | ΔThyA, HA9/10::fnr-DacA, HA3/4::PSynJ23119- |
| | | pKYNase, ATrpE |
| 3 | SYN4789 | ΔThyA, HA9/10::fnr-DacA, HA3/4::PSynJ23119- |
| | | pKYNase, ATrpE |
| 4 | SYN4737 | ΔΦ, ΔDAP, HA9/10::fnr-DacA |
| 5 | SYN4939 | ΔΦ, ΔDAP, ΔThyA, HA9/10::fnr-DacA, |
| | | PSynJ23119-pKYNase, ATrpE |
| 6 | | HA1/2::fnr-hcGAS |
| 7 | SYN5113 | ΔΦ, ΔDAP, ΔThyA, HA1/2::fnr-hcGAS |
| | | ΔΦ, ΔDAP, ΔThyA, HA9/10::fnr-DacA, |
| | | HA1/2::fnr-hcGAS |
| | | ΔΦ, ΔDAP, ΔthyA, HA9/10::fnr-DacA, |
| | | PsynJ23119-pKYNase, AtrpE,, HA1/2::fnr-hcGAS |

In order to generate the strains in Table 63, the methods described herein and known in the art were used in varying sequence. For example, SYN4910 was built from a Nissle strain that was wild type except for a phage3 knockout by adding DapA knockout, a HA910-FNR-dacA knockin and then a thyA knockout. In another example, SYN4939 was built from SYN2306 (a strain which comprises integrated kynureninase under control of a constitutive promoter) by adding a thyA knockout, HA910-FNR-dacA knockin, a dap knockout and a phage knockout.

### A. Chromosomal Integration of DacA

To generate strains comprising chromosomally integrated DacA under the control of the FNR promoter, dacA from Listeria monocytogenes was cloned into a KIKO vector under the control of the FNR promoter (SEQ ID NO: 1281). Knock-in PCR products were made from the KIKO vectors and allelic exchange was performed to integrate these operons into E. coli Nissle genome at the HA910 site.

Allelic exchange was facilitated through use of the lambda red recombinase system as described herein and in PCT/US2017/013072, filed Jan. 11, 2017, the contents of which is herein incorporated by reference in its entirety. The lambda red system is described in Datsenko and Wanner (One-step inactivation of chromosomal genes in Escherichia coli K-12 using PCR products; PNAS Jun. 6, 2000. 97 (12) 6640-6645) and modifications and adaptations thereof are also described in the art.

### B. Generation of a Phage 3 Knockout

Bioinformatic approaches helped identify 3 regions of the genome putatively containing active phage (as described in International Patent Application PCT/US18/38840, filed Jun. 21, 2018, the contents of which is herein incorporated by references in its entirety). Using an in-house developed PCR method, it was shown that the active phage originated from a genomic locus between bases 2,035,867 and 2,079,177 of the E. coli Nissle genome.

To inactivate the phage, lambda red recombineering was used to make a 9,687 base pair deletion. First, primers were designed and synthesized to amplify a chloramphenicol acetyltransferase (CAT) gene flanked by flippase recognition sites (FRT) from the plasmid pKD3. When introduced into Nissle, this cassette provides resistance to the antibiotic chloramphenicol. In addition, these primers contain 60 base pairs of homology to the genome which directs the antibiotic cassette into the Phage loci. The Phage3 KO FWD and Phage3 KO REV primers were used to PCR amplify a 1178 base pair linear DNA fragment, which was PCR purified. The resulting DNA template was used in recombineering.

To prepare for deletion of phage, first the lambda red system was introduced, by transforming a pKD46 plasmid comprising the lambda red genes into the E. coli Nissle host strain (wild type or comprising further engineered components) by electroporation as known in the art. The cells were spread out on a selective media plates and incubated overnight at 30° C. Next, the recombineering construct was transformed into the E. coli Nissle strain comprising pKD46 by electroporation. The transformed cells were spread out on an LB plate containing 35 μg/mL chloramphenicol and incubated overnight. The presence of the mutation was verified by colony PCR. The PCR product only forms if the CAT gene has inserted into the genome (thereby deleting and inactivating the Phage).

The antibiotic resistance gene was removed with the plasmid pCP20. Plasmid pCP20 is a temperature-sensitive plasmid that expresses the Flippase recombinase that will recombine the FRT-sites thereby removing the CAT gene. The strain with deleted phage sequence was grown in LB media containing antibiotics at 37° C. until it reached an OD600 of 0.4-0.6 and then transformed with pCP20 using electroporation. 200 ul of cells were spread on carbenicillin plates, 200 μL of cells were spread on chloramphenicol plates, and both were grown at 37° C. overnight. The carbenicillin plate contain cells with pCP20. The chloramphenicol plate provides an indication of how many cells survived the electroporation. Transformants from the carbenicillin plate were purified non-selectively at 43° C. and allowed to grow overnight.

The purified transformants were tested for sensitivity to carbenicillin and chloramphenicol. If no growth was observed on the chloramphenicol or carbenicillin plates for a colony, then both the CAT gene and the pCP20 plasmid were lost, and the colony was saved for further analysis. The saved colonies were restreaked onto an LB plate to obtain single colonies and grown overnight at 37° C. The deletion of the phage sequence was confirmed by sequencing the phage loci region of the genome and by phenotypically verifying the absence of plaque formation (essentially following the protocol as described in (as described in International Patent Application PCT/US18/38840, filed Jun. 21, 2018, the contents of which is herein incorporated by references in its entirety).

An alternative method of performing knockout such as phage KO is to use loxP recognition site instead of FRT and pCRE plasmid instead of pCP20. In this case, first, primers were designed and synthesized to amplify an aminoglycoside phosphotransferase (NeoR/KanR) gene flanked by flippase recognition sites (loxP) from the plasmid pKD4-loxP (SEQ ID NO: 1442). When introduced into Nissle, this cassette provides resistance to the antibiotic kanamycin. In addition, these primers contain 60 base pairs of homology to the genome which directs the antibiotic cassette into the Phage loci. The primers were used to PCR amplify a 1178 base pair linear DNA fragment, which was PCR purified. The resulting DNA template was used in recombineering.

To prepare for deletion of phage, first the lambda red system was introduced, by transforming a pKD46 plasmid comprising the lambda red genes into the E. coli Nissle host strain (wild type or comprising further engineered components) by electroporation as known in the art. The cells were spread out on a selective media plates and incubated overnight at 30° C. Next, the recombineering construct was transformed into the E. coli Nissle strain comprising pKD46 by electroporation. The transformed cells were spread out on an LB plate containing 50 μg/mL kanamycin and incubated overnight. The presence of the mutation was verified by colony PCR. The PCR product only forms if the CAT gene has inserted into the genome (thereby deleting and inactivating the Phage).

The antibiotic resistance gene was removed with the plasmid pKD4-loxP. Plasmid pKD4-loxP is an altered pKD4, in which the FRT sites are replaced by loxP sites, a temperature-sensitive plasmid that expresses the flippase recombinase that will recombine the loxP-sites thereby removing the NeoR/KanR gene. The strain with deleted phage sequence was grown in LB media containing antibiotics at 37° C. until it reached an OD600 of 0.4-0.6 and then transformed with logic1253 using electroporation. 200 ul of cells were spread on carbenicillin plates, 200 μL of cells were spread on kanamycin plates, and both were grown at 37° C. overnight. The carbenicillin plate contain cells with pKD4-loxP. The kanamycin plate provides an indication of how many cells survived the electroporation. Transformants from the carbenicillin plate were purified non-selectively at 43° C. and allowed to grow overnight.

The purified transformants were tested for sensitivity to carbenicillin and kanamycin. If no growth was observed on the kanamycin or carbenicillin plates for a colony, then both the NeoR/KanR gene and the pKD4-loxP plasmid were lost, and the colony was saved for further analysis. The saved colonies were restreaked onto an LB plate to obtain single colonies and grown overnight at 37° C. The deletion of the phage sequence was confirmed by PCR amplification of an expected, 640-bp fragment using primers (GTGATTAAGTACGTGAAATCGACTGAAC (SEQ ID NO: 1436) and CTCTGTCGGCAACATGAGGA (SEQ ID NO: 1437)) which flank the phage-deletion site.

Table 64. lists the Phage 3 genes that were inactivated by the deletion.

TABLE 64
Phage 3 Genes inactivated by the deletion

| | | | |
|---|---|---|---|
| ECOLIN_10110 | 1 . . . 160 | Minor tail protein U | GI: 660512026 |
| ECOLIN_10115 | 157 . . . 729 | tail protein | GI: 660512027 |
| ECOLIN_10120 | 745 . . . 987 | DNA breaking- | GI: 660512028 |
| | | rejoining protein | |
| ECOLIN_10125 | 1013 . . . 1339 | hypothetical protein | GI: 660512029 |
| ECOLIN_10130 | 1422 . . . 3368 | peptidase S14 | GI: 660512030 |
| ECOLIN_10135 | 3382 . . . 4881 | capsid protein | GI: 660512031 |
| ECOLIN_10140 | 4878 . . . 5093 | hypothetical protein | GI: 660512032 |
| ECOLIN_10145 | 5090 . . . 7192 | DNA packaging | GI: 660512033 |
| | | protein | |
| ECOLIN_10150 | 7192 . . . 7680 | terminase | GI: 660512034 |
| ECOLIN_10160 | 7864 . . . 8592 | hypothetical protein | GI: 660512035 |
| ECOLIN_10165 | 8767 . . . 8997 | hypothetical protein | GI: 660512036 |
| ECOLIN_10170 | 8996 . . . 9592 | hypothetical protein | GI: 660512037 |
| ECOLIN_10175 | 9661 . . . 9687 | hypothetical protein | GI: 660512038 |

C. Generation of a ThyA Auxotroph (ΔthyA) STING Agonist Producing Strain

Mutation of the thyA gene in E. coli Nissle yields a strain that cannot form a colony on LB plates unless they are supplemented with thymidine. A thyA::cam PCR fragment was amplified using 3 rounds of PCR with nested primers. For the first PCR round, pKD3 was used as template. The PCR product was purified from the remaining PCR reaction and used as template in the second PCR reaction. Again, the resulting PCR product was purified, and used as template in a third round of PCR. The resulting linear DNA fragment, which contained 92 bp homologous to upstream of thyA, the chloramphenicol cassette flanked by frt sites, and 98 bp homologous to downstream of the thyA gene, was transformed into a E. coli Nissle 1917 strain containing pKD46 grown for recombineering using electroporation. Colonies were selected on LB agar containing chloramphenicol (30 μg/mL) and thymidine (3 mM) and correct recombination events were verified by PCR. pKD46 was cured from the strain by passage at 37 C.

Next, the antibiotic resistance was removed with pCP20 transformation and cells were spread on LB agar plates containing 3 mM thymidine and 100 ug/ml carbenicillin and grown at 30° C. for 16-24 hours. Next, transformants were colony purified non-selectively (no antibiotics) at 42° C. To test the colony-purified transformants, a colonies were plated on Cam, Amp, and LB only plates and colonies were considered cured if there was no growth on the Cam or Amp plates. Absence of antibiotic cassettes was confirmed by PCR.

### D. Generation of a DAPA Auxotroph STING Strain

For creation of the dapA deletion (ΔdapA), two rounds of PCR were performed using nested primers. For the first round of PCR, pKD3 was used as the template DNA. The primers were designed to generate a dsDNA fragment that contained homology adjacent to the dapA gene locus in the EcN chromosome and a chloramphenicol resistance gene flanked by frt sites. The primers used in the second round of PCR utilized the PCR product of the first round as template DNA. These primers contained additional homology to dapA to provide a greater length of EcN homology for recombineering. The resulting dapA knockout fragment included 68 base pairs and 70 base pairs of EcN homology, respectively, on its 5′ and 3′ end. A strain which contains pKD46, was transformed with the dapA knockout fragment by electroporation. Colonies were selected on LB agar containing chloramphenicol (30 μg/mL) and diaminopimelate (100 μg/mL) and correct recombination events were verified by PCR. pKD46 was cured from the strain by passage at 37 C.

### Example 66. Construction and Activity of Various STING Agonist Producing Strains Under Control of a Low Oxygen Promoter

### Testing of Strains Comprising a Double Auxotrophy and a Phage Knockout

Strains comprising FNR-DacA and comprising a double auxotrophy and phage knockout, SYN4910 (ΔΦ, ΔDAP, ΔThyA, HA9/10::fnr-DacA) and combination strain SYN4939 (Nissle, ΔDAP, ΔThyA, HA9/10::fnr-DacA, PSynJ23119-pKYNase, ΔTrpE), were constructed according to methods described herein.

To measure compare in vitro activity of SYN4910 and SYN4939, overnight cultures of each strain including a Nissle control (SYN94, streptomycin resistant Nissle) were grown in LB at 37 C, shaking at 250 rpm. Cultures were back diluted 1:100 (10 mL in 125 mL baffled flask) and grown for 2-3 hours to early log phase. Once cultures reached early log, cultures were moved into a Coy anaerobic chamber supplying an anaerobic atmosphere (85% N2, 10% CO2, 5% H2). Cultures were incubated anaerobically for 3-4 hours or shaked at 37 C, 250 rpm overnight in a tube to allow for induction of the dacA gene.

Next, cultures were removed for LC/MS analysis of cyclic dinucleotide production. Samples were centrifuged for 5 minutes at 10000×g to separate cellular and extracellular fractions. Cell pellets were then used to determine intracellular cyclic-di-AMP. Concentrations were determined via LC/MS as described herein.

For quantifying kynurenine consumption, the LB overnight cultures SYN4910 and SYN4939 plus kynurenine consuming strain SYN2306 (comprising HA3/4::PSynJ23119-pKYNase delta TrpE) and Nissle control (SYN94) were diluted 1:25 in M9 minimal media supplemented with 0.5% glucose (w/v) and appropriate antibiotics or LB and antibiotics and grown shaking (250 rpm) at 37° C. for 5-6 hours. Cells were spun down and resuspended in LB with 200 μM L-kynurenine (Sigma-Aldrich®) to an optical density (600 nm) of 1.0. These cultures were then incubated at 37° C. with shaking for 3-4 hours to allow consumption of kynurenine. For kynurenine consumption measurements, samples were removed from each culture and spun for 5 minutes at 10000×g to separate the cellular and extracellular fractions. Media supernatant was removed and used to determine consumption of kynurenine via LC/MS analysis.

Results shown in FIG. 28B and FIG. 28C indicate both SYN4910 and SYN4939 were to produce cyclic-di-AMP. Results of FIG. 28D indicate that the combination strain SYN4939 also retained the ability to consume kynurenine similarly to it's parent SYN2306. Taken together, these results indicate that these strains comprising phage knockout and double auxotrophies maintain the ability of in vitro cyclic-di-AMP production and that the combination strain SYN4939 possesses the ability to both produce cyclic-di-AMP and consume kynurenine.

Testing of the effect of phage knockout and dapA auxotrophy on strain activity

To test the effect of phage knockout and dapA auxotrophy on strain activity, strains with and without phage knockout and dapA knockout, SYN4739 (Nissle HA3/4::PSynJ23119-pKYNase, ΔTrpE, ΔThyA, HA9/10::fnr-DacA) and SYN4939 (Nissle, ΔΦ, ΔDAP, ΔThyA, HA9/10::fnr-DacA, PSynJ23119-pKYNase, ΔTrpE), were compared side by side. Strains were grown essentially as described above assessed for c-di-AMP production and kynurenine consumption.

Cyclic-di-AMP production for both strains is shown in FIG. 29A and FIG. 29B. Kynurenine production as compared to kynurenine consumer single circuit strains SYN2028 and SYN2306 are shown in FIG. 29C and FIG. 29D Similar results can be seen in FIG. 30 and FIG. 31. SYN4787 essentially comprises the same circuitry as SYN4739 (Nissle ΔThyA, HA9/10::fnr-DacA, HA3/4::PSynJ23119-pKYNase, ΔTrpE) but does not any antibiotic markers (SYN4739 has kanamycin resistance). Taken together, these results indicate that the phage and dapA knockouts do not affect the activity of the strains in vitro.

### Example 67. Phage Testing Protocol: Plaque Assay of Bacterial Virus from Escherichia coli Using Mitomycin C Induction Data Analysis

The cell lines were analyzed for the production of phage using the mitomycin C phage induction procedure (Method STM-V-708, Plaque Assay of Bacterial Virus from Escherichia coli (E. coli). Using Mitomycin C Induction, as described in Sinsheimer RL. Purification and Properties of Bacteriophage X174. J. Mol. Biol. 1959; 1:37-42, and Clowes, R C and Hayes, W. Experiments in microbial genetics. John Wiley & Sons, N Y. 1968, the contents of each of which is herein incorporated by reference in its entireties). Briefly, sample (with thymidine supplemented media to support cell expansion, as appropriate) and control cells were grown overnight. A portion of the sample, positive control (E. coli, EMG 2: K (lambda), ATCC 23716, or equivalent) and negative control (E. coli, ATCC 13706, or equivalent) were removed and centrifuged, and each supernatant examined in a plaque assay for the presence of bacteriophage. Mitomycin C, at a final concentration of 2 ng/mL, was added to the remaining sample, positive and negative bacterial cultures. The cultures were then placed at 37±2° C. and shaken at 300-400 RPM until lysis occurred in the positive control (−4.5 hours). Each culture was treated with chloroform, centrifuged, and a 0.1 mL aliquot of the supernatant was examined for the presence of bacteriophage. To accomplish this, supernatants were mixed with phage-sensitive E. coli strain ATCC 13706, mixed with 0.7% agarose solution, and plated as a lawn atop lysogeny broth (LB) agar. The test was considered valid if plaques were present in the positive control and no plaques were present in the negative control.

### Example 68. Functional Assay for c-Di-AMP Produced by SYN4737

Next, a functional assay was performed to further confirm the activity of SYN4737 (delta Phage, delta Dap, HA9/10::FNR-dacA). The ability of SYN4737 to activate the STING pathway in antigen presenting cell populations was assessed. Bacteria (WT Nissle or SYN4737) were co-cultured at various multiplicities of infection (MOI) with 0.5×106 RAW 264.7 cells (immortalized murine macrophage cell line). SYN4737 was incubated shaking at 37 C, 250 rpm for 1 hour and induced for 4-5 hours in the anaerobe chamber prior to the experiment. Co-cultures were incubated for 4 hours as indicated and protein extracts were analyzed for IFNβ1 production. FIG. 32 shows that SYN4737, but not WT Nissle is able to induce IFNβ1 production in a manner dependent on the MOI.

1. A modified microorganism capable of producing at least one immune initiator and at least one immune sustainer.

2. The modified microorganism of claim 1, wherein the immune initiator is capable of enhancing oncolysis, activating antigen presenting cells (APCs), and/or priming and activating T cells.

3. The modified microorganism of claim 1 or claim 2, wherein the immune initiator is a therapeutic molecule encoded by at least one gene; a therapeutic molecule produced by an enzyme encoded by at least one gene; at least one enzyme of a biosynthetic or catabolic pathway encoded by at least one gene; at least one therapeutic molecule produced by at least one enzyme of a biosynthetic or catabolic pathway encoded by at least one gene; or a nucleic acid molecule that mediates RNA interference, microRNA response or inhibition, TLR response, antisense gene regulation, target protein binding, or gene editing.

4. The modified microorganism of any one of claims 1-3, wherein the immune initiator is a cytokine, a chemokine, a single chain antibody, a ligand, a metabolic converter, a T cell co-stimulatory receptor, a T cell co-stimulatory receptor ligand, an engineered chemotherapy, or a lytic peptide.

5. The modified microorganism of any one of claims 1-4, wherein the immune initiator is a STING agonist, arginine, 5-FU, TNFα, IFNγ, IFNβ1, agonistic anti-CD40 antibody, CD40L, SIRPα, GMCSF, agonistic anti-OXO40 antibody, OXO40L, agonistic anti-4-1BB antibody, 4-1BBL, agonistic anti-GITR antibody, GITRL, anti-PD1 antibody, anti-PDL1 antibody, or azurin.

6. The modified microorganism of claim 5, wherein the immune initiator is a STING agonist.

7. The modified microorganism of claim 6, wherein the STING agonist is c-diAMP, c-GAMP, or c-diGMP.

8. The modified microorganism of any one of claims 5-7, wherein the modified microorganism comprises at least one gene sequence encoding an enzyme which produces the immune initiator.

9. The modified microorganism of claim 8, wherein the at least one gene sequence encoding the immune initiator is a dacA gene sequence.

10. The modified microorganism of claim 8, wherein the at least one gene sequence encoding the immune initiator is a cGAS gene sequence.

11. The modified microorganism of claim 10, wherein the cGAS gene sequence is selected from a human cGAS gene sequence, a Verminephrobacter eiseniae cGAS gene sequence, Kingella denitrificans cGAS gene sequence, and a Neisseria bacilliformis cGAS gene sequence.

12. The modified microorganism of any one of claims 8-11, wherein the at least one gene sequence encoding the immune initiator is integrated into a chromosome of the modified microorganism or is present on a plasmid.

13. The modified microorganism of any one of claims 8-11, wherein the at least one gene sequence encoding the immune initiator is operably linked to an inducible promoter.

14. The modified microorganism of claim 13, wherein the inducible promoter is induced by low oxygen, anaerobic, or hypoxic conditions.

15. The modified microorganism of claim 5, wherein the immune initiator is arginine.

16. The modified microorganism of claim 15, wherein the microorganism comprises at least one gene sequence encoding at least one enzyme of the arginine biosynthetic pathway.

17. The modified microorganism of claim 15, wherein the at least one gene sequence encoding the at least one enzyme of the arginine biosynthetic pathway comprises feedback resistant argA.

18. The modified microorganism of claim 16, wherein the at least one gene sequence encoding the at least one enzyme of the arginine biosynthetic pathway is selected from the group consisting of: argA, argB, argC, argD, argE, argF, argG, argH, argI, argJ, carA, and carB.

19. The modified microorganism of claim 17 or claim 18, further comprising a deletion or a mutation in an arginine repressor gene (argR).

20. The modified microorganism of any one of claims 16-19, wherein the at least one gene sequence for the production of arginine is integrated into a chromosome of the modified microorganism or is present on a plasmid.

21. The modified microorganism of any one of claims 16-20, wherein the at least one gene sequence for the production of arginine is operably linked to an inducible promoter.

22. The modified microorganism of claim 21, wherein the inducible promoter is induced by low oxygen, anaerobic, or hypoxic conditions.

23. The modified microorganism of claim 5, wherein the immune initiator is 5-FU.

24. The modified microorganism of claim 23, wherein the microorganism comprises at least one gene sequence encoding an enzyme capable of converting 5-FC to 5-FU.

25. The modified microorganism of claim 24, wherein the at least one gene sequence is codA.

26. The modified microorganism of claim 24 or claim 25, wherein the at least one gene sequence is integrated into a chromosome of the modified microorganism or is present on a plasmid.

27. The modified microorganism of any one of claims 24-26, wherein the at least one gene sequence encoding the immune initiator is operably linked to an inducible promoter.

28. The modified microorganism of claim 27, wherein the inducible promoter is induced by low oxygen, anaerobic, or hypoxic conditions.

29. The modified microorganism of claim 1, wherein the immune sustainer is capable of enhancing trafficking and infiltration of T cells, enhancing recognition of cancer cells by T cells, enhancing effector T cell response, and/or overcoming immune suppression.

30. The modified microorganism of claim 1 or claim 29, wherein the immune sustainer is a therapeutic molecule encoded by at least one gene; a therapeutic molecule produced by an enzyme encoded by at least one gene; at least one enzyme of a biosynthetic or catabolic pathway encoded by at least one gene; at least one therapeutic molecule produced by at least one enzyme of a biosynthetic or catabolic pathway encoded by at least one gene; or a nucleic acid molecule that mediates RNA interference, microRNA response or inhibition, TLR response, antisense gene regulation, target protein binding, or gene editing.

31. The modified microorganism of any one of claim 1, 29, or 30, wherein the immune sustainer is a cytokine, a chemokine, a single chain antibody, a ligand, a metabolic converter, a T cell co-stimulatory receptor, or a T cell co-stimulatory receptor ligand.

32. The modified microorganism of any one of claim 1 or 29-31, wherein the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12.

33. The modified microorganism of claim 32, wherein the immune sustainer is a metabolic converter.

34. The modified microorganism of claim 33, wherein the metabolic converter is at least one enzyme of a kynurenine consumption pathway or at least one enzyme of an adenosine consumption pathway.

35. The modified microorganism of claim 33 or claim 34, wherein the microorganism comprises at least one gene sequence encoding the at least one enzyme of the kynurenine consumption pathway.

36. The modified microorganism of claim 35, wherein the at least one gene sequence encoding the at least one enzyme of the kynurenine consumption pathway is a kynureninase gene sequence.

37. The modified microorganism of claim 36, wherein the at least one gene sequence is kynU.

38. The modified microorganism of claim 37, wherein the at least one gene sequence is operably linked to a constitutive promoter.

39. The modified microorganism of any one of claims 35-38, wherein the at least one gene sequence encoding the at least one enzyme of the kynurenine consumption pathway is integrated into a chromosome of the microorganism or is present on a plasmid.

40. The modified microorganism of any one of claims 35-39, wherein the microorganism comprises a deletion or a mutation in trpE.

41. The modified microorganism of claim 32 or claim 33, wherein the microorganism comprises at least one gene sequence encoding at least one enzyme of an adenosine consumption pathway.

42. The modified microorganism of claim 41, wherein the at least one gene sequence encoding the at least one enzyme of the adenosine consumption pathway is selected from add, xapA, deoD, xdhA, xdhB, and xdhC.

43. The modified microorganism of claim 42, wherein the at least one gene sequence encoding the at least one enzyme of the adenosine consumption pathway is operably linked to a promoter induced by low oxygen, anaerobic, or hypoxic conditions.

44. The modified microorganism of any one of claims 41-43, wherein the at least one gene sequence encoding the at least one enzyme of the adenosine consumption pathway is integrated into a chromosome of the microorganism or is present on a plasmid.

45. The modified microorganism of any one of claims 41-44, wherein the modified microorganism comprises at least one gene sequence encoding an enzyme for importing adenosine into the microorganism.

46. The modified microorganism of claim 45, wherein the at least one gene sequence encoding the enzyme for importing adenosine into the microorganism is nupC or nupG.

47. The modified microorganism of claim 32 or claim 33, immune sustainer is arginine.

48. The modified microorganism of claim 47, wherein the microorganism comprises at least one gene sequence encoding at least one enzyme of the arginine biosynthetic pathway.

49. The modified microorganism of claim 48, wherein the at least one enzyme of the arginine biosynthetic pathway comprises feedback resistant argA.

50. The modified microorganism of claim 48 or claim 49, wherein the at least one gene sequence encoding the at least one enzyme of the arginine biosynthetic pathway is selected from the group consisting of: argA, argB, argC, argD, argE, argF, argG, argH, argI, argJ, carA, and carB.

51. The modified microorganism of any one of claims 48-50, wherein the at least one gene sequence encoding the at least one enzyme of the arginine biosynthetic pathway is operably linked to a promoter induced by low oxygen, anaerobic, or hypoxic conditions.

52. The modified microorganism of any one of claims 48-51, wherein the at least one gene sequence encoding the at least one enzyme of the arginine biosynthetic pathway is integrated into a chromosome of the modified microorganism or is present on a plasmid.

53. The modified microorganism of any one of claims 47-52, further comprising a deletion or a mutation in an arginine repressor gene (argR).

54. The modified microorganism of claim 32 or claim 33, wherein the immune sustainer is a STING agonist.

55. The modified microorganism of claim 54, wherein the STING agonist is c-diAMP, c-GAMP, or c-diGMP.

56. The modified microorganism of any one of claims 54-55, wherein the modified microorganism comprises at least one gene sequence encoding an enzyme which produces the STING agonist.

57. The modified microorganism of claim 56, wherein the at least one gene sequence encoding the immune sustainer is a dacA gene sequence.

58. The modified microorganism of claim 56, wherein the at least one gene sequence encoding the immune sustainer is a cGAS gene sequence.

59. The modified microorganism of claim 58, wherein the cGAS gene sequence is selected from a human cGAS gene sequence, a Verminephrobacter eiseniae cGAS gene sequence, Kingella denitrificans cGAS gene sequence, and a Neisseria bacilliformis cGAS gene sequence.

60. The modified microorganism of any one of the previous claims, wherein the modified microorganism is a bacterium or a yeast.

61. The modified microorganism of any one of the previous claims, wherein the modified microorganism is an E. coli bacterium.

62. The modified microorganism of any one of the previous claims, wherein the modified microorganism is an E. coli Nissle bacterium.

63. The modified microorganism of any one of the previous claims, wherein the modified microorganism comprises at least one mutation or deletion in a gene which results in one or more auxotrophies.

64. The modified microorganism of claim 63, wherein the at least one deletion or mutation is in a dapA gene and/or a thyA gene.

65. The modified microorganism of any one of the previous claims, comprising a phage deletion.

66. A composition comprising at least one modified microorganism capable of producing an immune initiator, and an immune sustainer.

67. The composition of claim 66, wherein the at least one modified microorganism is capable of producing the immune intiator and the immune sustainer.

68. The composition of claim 66, wherein the at least one modified microorganism is capable of producing the immune initiator, and at least a second modified microorganism is capable of producing the immune sustainer.

69. The composition of claim 66, wherein the immune sustainer is not produced by a modified microorganism in the composition.

70. A composition comprising at least one modified microorganism capable of producing an immune sustainer, and an immune initiator.

71. The composition of claim 70, wherein the at least one modified microorganism is capable of producing the immune initiator and the immune sustainer.

72. The composition of claim 70, wherein the at least one modified microorganism is capable of producing the immune sustainer, and at least a second modified microorganism is capable of producing the immune intiator.

73. The composition of claim 70, wherein the immune initiator is not produced by a modified microorganism in the composition.

74. A pharmaceutically acceptable composition comprising the modified microorganism of any one of claims 1-65 or the composition of any one of claims 66-73, and a pharmaceutically acceptable carrier.

75. The pharmaceutically acceptable composition of claim 74, wherein the composition is formulated for intratumoral administration.

76. A kit comprising the pharmaceutically acceptable composition of claim 74 or claim 75, and instructions for use thereof.

77. A method of treating cancer in a subject, the method comprising administering to the subject the pharmaceutically acceptable composition of claim 74 or claim 75, thereby treating cancer in the subject.

78. A method of inducing and sustaining an immune response in a subject, the method comprising administering to the subject the pharmaceutically acceptable composition of claim 74 or claim 75, thereby inducing and sustaining the immune response in the subject.

79. A method of inducing an abscopal effect in a subject having a tumor, the method comprising administering to the subject the pharmaceutically acceptable composition of claim 74 or claim 75, thereby inducing the abscopal effect in the subject.

80. A method of inducing immunological memory in a subject having a tumor, the method comprising administering to the subject the pharmaceutically acceptable composition of claim 74 or claim 75, thereby inducing the immunological memory in the subject.

81. A method of inducing partial regression of a tumor in a subject, the method comprising administering to the subject the pharmaceutically acceptable composition of claim 74 or claim 75, thereby inducing the partial regression of the tumor in the subject.

82. The method of claim 81, wherein the partial regression is a decrease in size of the tumor by at least about 10%, at least about 25%, at least about 50%, or at least about 75%.

83. A method of inducing complete regression of a tumor in a subject, the method comprising administering to the subject the pharmaceutically acceptable composition of claim 74 or claim 75, thereby inducing the complete regression of the tumor in the subject.

84. The method of claim 83, wherein the tumor is not detectable in the subject after administration of the pharmaceutically acceptable composition.

85. A method of treating cancer in a subject, the method comprising
administering a first modified microorganism to the subject, wherein the first modified microorganism is capable of producing an immune initiator; and
administering a second modified microorganism to the subject, wherein the second modified microorganism is capable of producing an immune sustainer,
thereby treating cancer in the subject.

86. A method of inducing and sustaining an immune response in a subject, the method comprising
administering a first modified microorganism to the subject, wherein the first modified microorganism is capable of producing an immune initiator; and
administering a second modified microorganism to the subject, wherein the second modified microorganism is capable of producing an immune sustainer,
thereby inducing and sustaining the immune response in the subject.

87. The method of claim 85 or claim 86, wherein the administering steps are performed at the same time; wherein administering of the first modified microorganism to the subject occurs before administering of the second modified microorganism to the subject; or wherein administering of the second modified microorganism to the subject occurs before administering of the first modified microorganism to the subject.

88. A method of treating cancer in a subject, the method comprising
administering a first modified microorganism to the subject, wherein the first modified microorganism is capable of producing an immune initiator; and
administering an immune sustainer to the subject,
thereby treating cancer in the subject.

89. A method of inducing and sustaining an immune response in a subject, the method comprising
administering a first modified microorganism to the subject, wherein the first modified microorganism is capable of producing an immune initiator; and
administering an immune sustainer to the subject,
thereby inducing and sustaining the immune response in the subject.

90. The method of claim 88 or claim 89, wherein the administering steps are performed at the same time; wherein administering of the first modified microorganism to the subject occurs before administering of the immune sustainer to the subject; or wherein administering of the immune sustainer to the subject occurs before administering of the first modified microorganism to the subject.

91. A method of treating cancer in a subject, the method comprising
administering an immune initiator to the subject; and
administering a first modified microorganism to the subject, wherein the first modified microorganism is capable of producing an immune sustainer,
thereby treating cancer in the subject.

92. A method of inducing and sustaining an immune response in a subject, the method comprising
administering an immune initiator to the subject; and
administering a first modified microorganism to the subject, wherein the first modified microorganism is capable of producing an immune sustainer,
thereby inducing and sustaining the immune response in the subject.

93. The method of claim 91 or claim 92, wherein the administering steps are performed at the same time; wherein the administering of the first modified microorganism to the subject occurs before the administering of the immune initiator to the subject; or wherein the administering of the immune initiator to the subject occurs before the administering of the first modified microorganism to the subject.

94. The method of any one of claims 77-93, wherein the administering is intratumoral injection.
